SEQUENCE LISTING (36 sequences)

SEQ ID NO: 1 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 2 - protein, 1271 aa, Homo sapiens
MVDPVGFAEAWKAQFPDSEPPRMELRSVGDIEQELERCKASIRRLEQEVNQERFRMIYLQTLLAKEKKSYDRQRWGFRRAAQAPDGASEPRASASRPQPAPADGADPPPAEEPEARPDGEGSPGKARPGTARRPGAAASGERDDRGPPASVAALRSNFERIRKGHGQPGADAEKPFYVNVEFHHERGLVKVNDKEVSDRISSLGSQAMQMERKKSQHGAGSSVGDASRPPYRGRSSESSCGVDGDYEDAELNPRFLKDNLIDANGGSRPPWPPLEYQPYQSIYVGGMMEGEGKGPLLRSQSTSEQEKRLTWPRRSYSPRSFEDCGGGYTPDCSSNENLTSSEEDFSSGQSSRVSPSPTTYRMFRDKSRSPSQNSQQSFDSSSPPTPQCHKRHRHCPVVVSEATIVGVRKTGQIWPNDGEGAFHGDADGSFGTPPGYGCAADRAEEQRRHQDGLPYIDDSPSSSPHLSSKGRGSRDALVSGALESTKASELDLEKGLEMRKWVLSGILASEETYLSHLEALLLPMKPLKAAATTSQPVLTSQQIETIFFKVPELYEIHKEFYDGLFPRVQQWSHQQRVGDLFQKLASQLGVYRAFVDNYGVAMEMAEKCCQANAQFAEISENLRARSNKDAKDPTTKNSLETLLYKPVDRVTRSTLVLHDLLKHTPASHPDHPLLQDALRISQNFLSSINEEITPRRQSMTVKKGEHRQLLKDSFMVELVEGARKLRHVFLFTDLLLCTKLKKQSGGKTQQYDCKWYIPLTDLSFQMVDELEAVPNIPLVPDEELDALKIKISQIKNDIQREKRANKGSKATERLKKKLSEQESLLLLMSPSMAFRVHSRNGKSYTFLISSDYERAEWRENIREQQKKCFRSFSLTSVELQMLTNSCVKLQTVHSIPLTINKEDDESPGLYGFLNVIVHSATGFKQSSNLYCTLEVDSFGYFVNKAKTRVYRDTAEPNWNEEFEIELEGSQTLRILCYEKCYNKTKIPKEDGESTDRLMGKGQVQLDPQALQDRDWQRTVIAMNGIEVKLSVKFNSREFSLKRMPSRKQTGVFGVKIAVVTKRERSKVPYIVRQCVEEIERRGMEEVGIYRVSGVATDIQALKAAFDVNNKDVSVMMSEMDVNAIAGTLKLYFRELPEPLFTDEFYPNFAEGIALSDPVAKESCMLNLLLSLPEANLLTFLFLLDHLKRVAEKEAVNKMSLHNLATVFGPTLLRPSEKESKLPANPSQPITMTDSWSLEVMSQVQVLLYFLQLEAIPAPDSKRQSILFSTEV

SEQ ID NO: 3 - protein, 1210 aa, Homo sapiens [sequence omitted]

SEQ ID NO: 4 - protein, 1255 aa, Homo sapiens [sequence omitted]

SEQ ID NO: 5 - protein, 671 aa, Homo sapiens
MADPAAGPPPSEGEESTVRFARKGALRQKNVHEVKNHKFTARFFKQPTFCSHCTDFIWGFGKQGFQCQVCCFVVHKRCHEFVTFSCPGADKGPASDDPRSKHKFKIHTYSSPTFCDHCGSLLYGLIHQGMKCDTCMMNVHKRCVMNVPSLCGTDHTERRGRIYIQAHIDRDVLIVLVRDAKNLVPMDPNGLSDPYVKLKLIPDPKSESKQKTKTIKCSLNPEWNETFRFQLKESDKDRRLSVEIWDWDLTSRNDFMGSLSFGISELQKASVDGWFKLLSQEEGEYFNVPVPPEGSEANEELRQKFERAKISQGTKVPEEKTTNTVSKFDNNGNRDRMKLTDFNFLMVLGKGSFGKVMLSERKGTDELYAVKILKKDVVIQDDDVECTMVEKRVLALPGKPPFLTQLHSCFQTMDRLYFVMEYVNGGDLMYHIQQVGRFKEPHAVFYAAEIAIGLFFLQSKGIIYRDLKLDNVMLDSEGHIKIADFGMCKENIWDGVTTKTFCGTPDYIAPEIIAYQPYGKSVDWWAFGVLLYEMLAGQAPFEGEDEDELFQSIMEHNVAYPKSMSKEAVAICKGLMTKHPGKRLGCGPEGERDIKEHAFFRYIDWEKLERKEIQPPYKPKARDKRDTSNFDKEFTRQPVELTPTDKLFIMNLDQNEFAGFSYTNPEFVINV

SEQ ID NO: 6 - protein, 706 aa, Homo sapiens
MSPFLRIGLSNFDCGSCQSCQGEAVNPYCAVLVKEYVESENGQMYIQKKPTMYPPWDSTFDAHINKGRVMQIIVKGKNVDLISETTVELYSLAERCRKNNGKTEIWLELKPQGRMLMNARYFLEMSDTKDMNEFETEGFFALHQRRGAIKQAKVHHVKCHEFTATFFPQPTFCSVCHEFVWGLNKQGYQCRQCNAAIHKKCIDKVIAKCTGSAINSRETMFHKERFKIDMPHRFKVYNYKSPTFCEHCGTLLWGLARQGLKCDACGMNVHHRCQTKVANLCGINQKLMAEALAMIESTQQARCLRDTEQIFREGPVEIGLPCSIKNEARPPCLPTPGKREPQGISWESPLDEVDKMCHLPEPELNKERPSLQIKLKIEDFILHKMLGKGSFGKVFLAEFKKTNQFFAIKALKKDVVLMDDDVECTMVEKRVLSLAWEHPFLTHMFCTFQTKENLFFVMEYLNGGDLMYHIQSCHKFDLSRATFYAAEIILGLQFLHSKGIVYRDLKLDNILLDKDGHIKIADFGMCKENMLGDAKTNTFCGTPDYIAPEILLGQKYNHSVDWWSFGVLLYEMLIGQSPFHGQDEEELFHSIRMDNPFYPRWLEKEAKDLLVKLFVREPEKRLGVRGDIRQHPLFREINWEELERKEIDPPFRPKVKSPFDCSNFDKEFLNEKPRLSFADRALINSMDQNMFRNFSFMNPGMERLIS

SEQ ID NO: 7 - protein, 824 aa, Homo sapiens
MSLLGDPLQALPPSAAPTGPLLAPPAGATLNRLREPLLRRLSELLDQAPEGRGWRRLAELAGSRGRLRLSCLDLEQCSLKVLEPEGSPSLCLLKLMGEKGCTVTELSDFLQAMEHTEVLQLLSPPGIKITVNPESKAVLAGQFVKLCCRATGHPFVQYQWFKMNKEIPNGNTSELIFNAVHVKDAGFYVCRVNNNFTFEFSQWSQLDVCDIPESFQRSVDGVSESKLQICVEPTSQKLMPGSTLVLQCVAVGSPIPHYQWFKNELPLTHETKKLYMVPYVDLEHQGTYWCHVYNDRDSQDSKKVEIIIGRTDEAVECTEDELNNLGHPDNKEQTTDQPLAKDKVALLIGNMNYREHPKLKAPLVDVYELTNLLRQLDFKVVSLLDLTEYEMRNAVDEFLLLLDKGVYGLLYYAGHGYENFGNSFMVPVDAPNPYRSENCLCVQNILKLMQEKETGLNVFLLDMCRKRNDYDDTIPILDALKVTANIVFGYATCQGAEAFEIQHSGLANGIFMKFLKDRLLEDKKITVLLDEVAEDMGKCHLTKGKQALEIRSSLSEKRALTDPIQGTEYSAESLVRNLQWAKAHELPESMCLKFDCGVQIQLGFAAEFSNVMIIYTSIVYKPPEIIMCDAYVTDFPLDLDIDPKDANKGTPEETGSYLVSKDLPKHCLYTRLSSLQKLKEHLVFTVCLSYQYSGLEDTVEDKQEVNVGKPLIAKLDMHRGLGRKTCFQTCLMSNGPYQSSAATSGGAGHYHSLQDPFHGVYHSHPGNPSNVTPADSCHCSRTPDAFISSFAHHASCHFSRSNVPVETTDEIPFSFSDRLRISEK

SEQ ID NO: 8 - protein, 1154 aa, Homo sapiens [sequence omitted]

SEQ ID NO: 9 - protein, 1004 aa, Homo sapiens
MGELCRRDSALTALDEETLWEMMESHRHRIVRCICPSRLTPYLRQAKVLCQLDEEEVLHSPRLTNSAMRAGHLLDLLKTRGKNGAIAFLESLKFHNPDVYTLVTGLQPDVDFSNFSGLMETSKLTECLAGAIGSLQEELNQEKGQKEVLLRRCQQLQEHLGLAETRAEGLHQLEADHSRMKREVSAHFHEVLRLKDEMLSLSLHYSNALQEKELAASRCRSLQEELYLLKQELQRANMVSSCELELQEQSLRTASDQESGDEELNRLKEENEKLRSLTFSLAEKDILEQSLDEARGSRQELVERIHSLRERAVAAERQREQYWEEKEQTLLQFQKSKMACQLYREKVNALQAQVCELQKERDQAYSARDSAQREISQSLVEKDSLRRQVFELTDQVCELRTQLRQLQAEPPGVLKQEARTREPCPREKQRLVRMHAICPRDDSDCSLVSSTESQLLSDLSATSSRELVDSFRSSSPAPPSQQSLYKRVAEDFGEEPWSFSSCLEIPEGDPGALPGAKAGDPHLDYELLDTADLPQLESSLQPVSPGRLDVSESGVLMRRRPARRILSQVTMLAFQGDALLEQISVIGGNLTGIFIHRVTPGSAADQMALRPGTQIVMVDYEASEPLFKAVLEDTTLEEAVGLLRRVDGFCCLSVKVNTDGYKRLLQDLEAKVATSGDSFYIRVNLAMEGRAKGELQVHCNEVLHVTDTMFQGCGCWHAHRVNSYTMKDTAAHGTIPNYSRAQQQLIALIQDMTQQCTVTRKPSSGGPQKLVRIVSMDKAKASPLRLSFDRGQLDPSRMEGSSTCFWAESCLTLVPYTLVRPHRPARPRPVLLVPRAVGKILSEKLCLLQGFKKCLAEYLSQEEYEAWSQRGDIIQEGEVSGGRCWVTRHAVESLMEKNTHALLDVQLDSVCTLHRMDIFPIVIHVSVNEKMAKKLKKGLQRLGTSEEQLLEAARQEEGDLDRAPCLYSSLAPDGWSDLDGLLSCVRQAIADEQKKVVWTEQSPR

SEQ ID NO: 10 - protein, 1032 aa, Homo sapiens
MPGRAEAGEAEEEAGAGSGSEAEEDALWERIEGVRHRLARALNPAKLTPYLRQCRVIDEQDEEEVLSTYRFPCRVNRTGRLMDILRCRGKRGYEAFLEALEFYYPEHFTLLTGQEPAQRCSMILDEEGPEGLTQFLMTEVRRLREARKSQLQREQQLQARGRVLEEERAGLEQRLRDQQQAQERCQRLREDWEAGSLELLRLKDENYMIAMRLAQLSEEKNSAVLRSRDLQLAVDQLKLKVSRLEEECALLRRARGPPPGAEEKEKEKEKEKEPDNVDLVSELRAENQRLTASLRELQEGLQQEASRPGAPGSERILLDILEHDWREAQDSRQELCQKLHAVQGELQWAEELRDQYLQEMEDLRLKHRTLQKDCDLYKHRMATVLAQLEEIEKERDQAIQSRDRIQLQYSQSLIEKDQYRKQVRGLEAERDELLTTLTSLEGTKALLEVQLQRAQGGTCLKACASSHSLCSNLSSTWSLSEFPSPLGGPEATGEAAVMGGPEPHNSEEATDSEKEINRLSILPFPPSAGSILRRQREEDPAPPKRSFSSMSDITGSVTLKPWSPGLSSSSSSDSVWPLGKPEGLLARGCGLDFLNRSLAIRVSGRSPPGGPEPQDKGPDGLSFYGDRWSGAVVRRVLSGPGSARMEPREQRVEAAGLEGACLEAEAQQRTLLWNQGSTLPSLMDSKACQSFHEALEAWAKGPGAEPFYIRANLTLPERADPHALCVKAQEILRLVDSAYKRRQEWFCTRVDPLTLRDLDRGTVPNYQRAQQLLEVQEKCLPSSRHRGPRSNLKKRALDQLRLVRPKPVGAPAGDSPDQLLLEPCAEPERSLRPYSLVRPLLVSALRPVVLLPECLAPRLIRNLLDLPSSRLDFQVCPAESLSGEELCPSSAPGAPKAQPATPGLGSRIRAIQESVGKKHCLLELGARGVRELVQNEIYPIVIHVEVTEKNVREVRGLLGRPGWRDSELLRQCRGSEQVLWGLPCSWVQVPAHEWGHAEELAKVVRGRILQEQARLVWVECGSSRGCPSSSEA

SEQ ID NO: 11 - protein, 536 aa, Homo sapiens
MSDYENDDECWSVLEGFRVTLTSVIDPSRITPYLRQCKVLNPDDEEQVLSDPNLVIRKRKVGVLLDILQRTGHKGYVAFLESLELYYPQLYKKVTGKEPARVFSMIIDASGESGLTQLLMTEVMKLQKKVQDLTALLSSKDDFIKELRVKDSLLRKHQERVQRLKEECEAGSRELKRCKEENYDLAMRLAHQSEEKGAALMRNRDLQLEIDQLKHSLMKAEDDCKVERKHTLKLRHAMEQRPSQELLWELQQEKALLQARVQELEASVQEGKLDRSSPYIQVLEEDWRQALRDHQEQANTIFSLRKDLRQGEARRLRCMEEKEMFELQCLALRKDSKMYKDRIEAILLQMEEVAIERDQAIATREELHAQHARGLQEKDALRKQVRELGEKADELQLQVFQCEAQLLAVEGRLRRQQLETLVLSSDLEDGSPRRSQELSLPQDLEDTQLSDKGCLAGGGSPKQPFAALHQEQVLRNPHDAGLSSGEPPEKERRRLKESFENYRRKRALRKMQKGWRQGEEDRENTTGSDNTDTEGS

SEQ ID NO: 12 - protein, 233 aa, Homo sapiens
MEPTAPSLTEEDLTEVKKDALENLRVYLCEKIIAERHFDHLRAKKILSREDTEEISCRTSSRKRAGKLLDYLQENPKGLDTLVESIRREKTQNFLIQKITDEVLKLRNIKLEHLKGLKCSSCEPFPDGATNNLSRSNSDESNFSEKLRASTVMYHPEGESSTTPFFSTNSSLNLPVLEVGRTENTIFSSTTLPRPGDPGAPPLPPDLQLEEEGTCANSSEMFLPLRSRTVSRQ

SEQ ID NO: 13 - protein, 790 aa, Homo sapiens
MAEQVLPQALYLSNMRKAVKIRERTPEDIFKPTNGIIHHFKTMHRYTLEMFRTCQFCPQFREIIHKALIDRNIQATLESQKKLNWCREVRKLVALKTNGDGNCLMHATSQYMWGVQDTDLVLRKALFSTLKETDTRNFKFRWQLESLKSQEFVETGLCYDTRNWNDEWDNLIKMASTDTPMARSGLQYNSLEEIHIFVLCNILRRPIIVISDKMLRSLESGSNFAPLKVGGIYLPLHWPAQECYRYPIVLGYDSHHFVPLVTLKDSGPEIRAVPLVNRDRGRFEDLKVHFLTDPENEMKEKLLKEYLMVIEIPVQGWDHGTTHLINAAKLDEANLPKEINLVDDYFELVQHEYKKWQENSEQGRREGHAQNPMEPSVPQLSLMDVKCETPNCPFFMSVNTQPLCHECSERRQKNQNKLPKLNSKPGPEGLPGMALGASRGEAYEPLAWNPEESTGGPHSAPPTAPSPFLFSETTAMKCRSPGCPFTLNVQHNGFCERCHNARQLHASHAPDHTRHLDPGKCQACLQDVTRTFNGICSTCFKRTTAEASSSLSTSLPPSCHQRSKSDPSRLVRSPSPHSCHRAGNDAPAGCLSQAARTPGDRTGTSKCRKAGCVYFGTPENKGFCTLCFIEYRENKHFAAASGKVSPTASRFQNTIPCLGRECGTLGSTMFEGYCQKCFIEAQNQRFHEAKRTEEQLRSSQRRDVPRTTQSTSRPKCARASCKNILACRSEELCMECQHPNQRMGPGAHRGEPAPEDPPKQRCRAPACDHFGNAKCNGYCNECFQFKQMYG

SEQ ID NO: 14 - protein, 956 aa, Homo sapiens
MSSGLWSQEKVTSPYWEERIFYLLLQECSVTDKQTQKLLKVPKGSIGQYIQDRSVGHSRIPSAKGKKNQIGLKILEQPHAVLFVDEKDVVEINEKFTELLLAITNCEERFSLFKNRNRLSKGLQIDVGCPVKVQLRSGEEKFPGVVRFRGPLLAERTVSGIFFGVELLEEGRGQGFTDGVYQGKQLFQCDEDCGVFVALDKLELIEDDDTALESDYAGPGDTMQVELPPLEINSRVSLKVGETIESGTVIFCDVLPGKESLGYFVGVDMDNPIGNWDGRFDGVQLCSFACVESTILLHINDIIPALSESVTQERRPPKLAFMSRGVGDKGSSSHNKPKATGSTSDPGNRNRSELFYTLNGSSVDSQPQSKSKNTWYIDEVAEDPAKSLTEISTDFDRSSPPLQPPPVNSLTTENRFHSLPFSLTKMPNTNGSIGHSPLSLSAQSVMEELNTAPVQESPPLAMPPGNSHGLEVGSLAEVKENPPFYGVIRWIGQPPGLNEVLAGLELEDECAGCTDGTFRGTRYFTCALKKALFVKLKSCRPDSRFASLQPVSNQIERCNSLAFGGYLSEVVEENTPPKMEKEGLEIMIGKKKGIQGHYNSCYLDSTLFCLFAFSSVLDTVLLRPKEKNDVEYYSETQELLRTEIVNPLRIYGYVCATKIMKLRKILEKVEAASGFTSEEKDPEEFLNILFHHILRVEPLLKIRSAGQKVQDCYFYQIFMEKNEKVGVPTIQQLLEWSFINSNLKFAEAPSCLIIQMPRFGKDFKLFKKIFPSLELNITDLLEDTPRQCRICGGLAMYECRECYDDPDISAGKIKQFCKTCNTQVHLHPKRLNHKYNPVSLPKDLPDWDWRHGCIPCQNMELFAVLCIETSHYVAFVKYGKDDSAWLFFDSMADRDGGQNGFNIPQVTPCPEVGEYLKMSLEDLHSLDSRRIQGCARRLLCDAYMCMYQSPTMSLYK

SEQ ID NO: 15 - protein, 579 aa, Homo sapiens
MLRSGPASGPSVPTGRAMPSRRVARPPAAPELGALGSPDLSSLSLAVSRSTDELEIIDEYIKENGFGLDGGQPGPGEGLPRLVSRGAASLSTVTLGPVAPPATPPPWGCPLGRLVSPAPGPGPQPHLVITEQPKQRGMRFRYECEGRSAGSILGESSTEASKTLPAIELRDCGGLREVEVTACLVWKDWPHRVHPHSLVGKDCTDGICRVRLRPHVSPRHSFNNLGIQCVRKKEIEAAIERKIQLGIDPYNAGSLKNHQEVDMNVVRICFQASYRDQQGQMRRMDPVLSEPVYDKKSTNTSELRICRINKESGPCTGGEELYLLCDKVQKEDISVVFSRASWEGRADFSQADVHRQIAIVFKTPPYEDLEIVEPVTVNVFLQRLTDGVCSEPLPFTYLPRDHDSYGVDKKRKRGMPDVLGELNSSDPHGIESKRRKKKPAILDHFLPNHGSGPFLPPSALLPDPDFFSGTVSLPGLEPPGGPDLLDDGFAYDPTAPTLFTMLDLLPPAPPHASAVVCSGGAGAVVGETPGPEPLTLDSYQAPGPGDGGTASLVGSNMFPNHYREAAFGGGLLSPGPEAT

SEQ ID NO: 16 - protein, 599 aa, Homo sapiens
MSGPCGEKPVLEASPTMSLWEFEDSHSRQGTPRPGQELAAEEASALELQMKVDFFRKLGYSSTEIHSVLQKLGVQADTNTVLGELVKHGTATERERQTSPDPCPQLPLVPRGGGTPKAPNLEPPLPEEEKEGSDLRPVVIDGSNVAMSHGNKEVFSCRGILLAVNWFLERGHTDITVFVPSWRKEQPRPDVPITDQHILRELEKKKILVFTPSRRVGGKRVVCYDDRFIVKLAYESDGIVVSNDTYRDLQGERQEWKRFIEERLLMYSFVNDKFMPPDDPLGRHGPSLDNFLRKKPLTLEHRKQPCPYGRKCTYGIKCRFFHPERPSCPQRSVADELRANALLSPPRAPSKDKNGRRPSPSSQSSSLLTESEQCSLDGKKLGAQASPGSRQEGLTQTYAPSGRSLAPSGGSGSSFGPTDWLPQTLDSLPYVSQDCLDSGIGSLESQMSELWGVRGGGPGEPGPPRAPYTGYSPYGSELPATAAFSAFGRAMGAGHFSVPADYPPAPPAFPPREYWSEPYPLPPPTSVLQEPPVQSPGAGRSPWGRAGSLAKEQASVYTKLCGVFPPHLVEAVMGRFPQLLDPQQLAAEILSYKSQHPSE

SEQ ID NO: 17 - protein, 1133 aa, Homo sapiens
MPVQAPQWTDFLSCPICTQTFDETIRKPISLGCGHTVCKMCLNKLHRKACPFDQTTINTDIELLPVNSALLQLVGAQVPEQQPITLCSGVEDTKHYEEAKKCVEELALYLKPLSSARGVGLNSTTQSVLSRPMQRKLVTLVHCQLVEEEGRIRAMRAARSLGERTVTELILQHQNPQQLSSNLWAAVRARGCQFLGPAMQEEALKLVLLALEDGSALSRKVLVLFVVQRLEPRFPQASKTSIGHVVQLLYRASCFKVTKRDEDSSLMQLKEEFRTYEALRREHDSQIVQIAMEAGLRIAPDQWSSLLYGDQSHKSHMQSIIDKLQTPASFAQSVQELTIALQRTGDPANLNRLRPHLELLANIDPSPDAPPPTWEQLENGLVAVRTVVHGLVDYIQNHSKKGADQQQPPQHSKYKTYMCRDMKQRGGCPRGASCTFAHSQEELEKFRKMNKRLVPRRPLSASLGQLNEVGLPSAAILPDEGAVDLPSRKPPALPNGIVSTGNTVTQLIPRGTDPSYDSSLKPGKIDHLSSSAPGSPPDLLESVPKSISALPVNPHSIPPRGPADLPPMPVTKPLQMVPRGSQLYPAQQTDVYYQDPRGAAPPFEPAPYQQGMYYTPPPQCVSRFVRPPPSAPEPAPPYLDHYPPYLQERVVNSQYGTQPQQYPPIYPSHYDGRRVYPAPSYTREEIFRESPIPIEIPPAAVPSYVPESRERYQQIESYYPVAPHPTQIRPSYLREPPYSRLPPPPQPHPSLDELHRRRKEIMAQLEERKVISPPPFAPSPTLPPTFHPEEFLDEDLKVAGKYKGNDYSQYSPWSCDTIGSYIGTKDAKPKDVVAAGSVEMMNVESKGMRDQRLDLQRRAAETSDDDLIPFGDRPTVSRFGAISRTSKTIYQGAGPMQAMAPQGAPTKSINISDYSPYGTHGGWGASPYSPHQNIPSQGHFSERERISMSEVASHGKPLPSAEREQLRLELQQLNHQISQQTQLRGLEAVSNRLVLQREANTLAGQSQPPPPPPPKWPGMISSEQLSLELHQVEREIGKRTRELSMENQCSLDMKSKLNTSKQAENGQPEPQNKVPAEDLTLTFSDVPNGSALTQENISLLSNKTSSLNLSEDPEGGGDNNDSQRSGVTPSSAP

SEQ ID NO: 18 - protein, 510 aa, Homo sapiens
MDEKTKKAEEMALSLTRAVAGGDEQVAMKCAIWLAEQRVPLSVQLKPEVSPTQDIRLWVSVEDAQMHTVTIWLTVRPDMTVASLKDMVFLDYGFPPVLQQWVIGQRLARDQETLHSHGVRQNGDSAYLYLLSARNTSLNPQELQRERQLRMLEDLGFKDLTLQPRGPLEPGPPKPGVPQEPGRGQPDAVPEPPPVGWQCPGCTFINKPTRPGCEMCCRARPEAYQVPASYQPDEEERARLAGEEEALRQYQQRKQQQQEGNYLQHVQLDQRSLVLNTEPAECPVCYSVLAPGEAVVLRECLHTFCRECLQGTIRNSQEAEVSCPFIDNTYSCSGKLLEREIKALLTPEDYQRFLDLGISIAENRSAFSYHCKTPDCKGWCFFEDDVNEFTCPVCFHVNCLLCKAIHEQMNCKEYQEDLALRAQNDVAARQTTEMLKVMLQQGEAMRCPQCQIVVQKKDGCDWIRCTVCHTEICWVTKGPRWGPGGPGDTSGGCRCRVNGIPCHPSCQNCH

SEQ ID NO: 19 - protein, 947 aa, Homo sapiens
MAVMEMACPGAPGSAVGQQKELPKAKEKTPPLGKKQSSVYKLEAVEKSPVFCGKWEILNDVITKGTAKEGSEAGPAAISIIAQAECENSQEFSPTFSERIFIAGSKQYSQSESLDQIPNNVAHATEGKMARVCWKGKRRSKARKKRKKKSSKSLAHAGVALAKPLPRTPEQESCTIPVQEDESPLGAPYVRNTPQFTKPLKEPGLGQLCFKQLGEGLRPALPRSELHKLISPLQCLNHVWKLHHPQDGGPLPLPTHPFPYSRLPHPFPFHPLQPWKPHPLESFLGKLACVDSQKPLPDPHLSKLACVDSPKPLPGPHLEPSCLSRGAHEKFSVEEYLVHALQGSVSSGQAHSLTSLAKTWAARGSRSREPSPKTEDNEGVLLTEKLKPVDYEYREEVHWATHQLRLGRGSFGEVHRMEDKQTGFQCAVKKVRLEVFRAEELMACAGLTSPRIVPLYGAVREGPWVNIFMELLEGGSLGQLVKEQGCLPEDRALYYLGQALEGLEYLHSRRILHGDVKADNVLLSSDGSHAALCDFGHAVCLQPDGLGKSLLTGDYIPGTETHMAPEVVLGRSCDAKVDVWSSCCMMLHMLNGCHPWTQFFRGPLCLKIASEPPPVREIPPSCAPLTAQAIQEGLRKEPIHRVSAAELGGKVNRALQQVGGLKSPWRGEYKEPRHPPPNQANYHQTLHAQPRELSPRAPGPRPAEETTGRAPKLQPPLPPEPPEPNKSPPLTLSKEESGMWEPLPLSSLEPAPARNPSSPERKATVPEQELQQLEIELFLNSLSQPFSLEEQEQILSCLSIDSLSLSDDSEKNPSKASQSSRDTLSSGVHSWSSQAEARSSSWNMVLARGRPTDTPSYFNGVKVQIQSLNGEHLHIREFHRVKVGDIATGISSQIPAAAFSLVTKDGQPVRYDMEVPDSGIDLQCTLAPDGSFAWSWRVKHGQLENRP

SEQ ID NO: 20 - protein, 599 aa, Homo sapiens
MENCLGESRHEVEKSEISENTDASGKIEKYNVPLNRLKMMFEKGEPTQTKILRAQSRSASGRKISENSYSLDDLEIGPGQLSSSTFDSEKNESRRNLELPRLSETSIKDRMAKYQAAVSKQSSSTNYTNELKASGGEIKIHKMEQKENVPPGPEVCITHQEGEKISANENSLAVRSTPAEDDSRDSQVKSEVQQPVHPKPLSPDSRASSLSESSPPKAMKKFQAPARETCVECQKTVYPMERLLANQQVFHISCFRCSYCNNKLSLGTYASLHGRIYCKPHFNQLFKSKGNYDEGFGHRPHKDLWASKNENEEILERPAQLANARETPHSPGVEDAPIAKVGVLAASMEAKASSQQEKEDKPAETKKLRIAWPPPTELGSSGSALEEGIKMSKPKWPPEDEISKPEVPEDVDLDLKKLRRSSSLKERSRPFTVAASFQSTSVKSPKTVSPPIRKGWSMSEQSEESVGGRVAERKQVENAKASKKNGNVGKTTWQNKESKGETGKRSKEGHSLEMENENLVENGADSDEDDNSFLKQQSPQEPKSLNWSSFVDNTFAEEFTTQNQKSQDVELWEGEVVKELSVEEQIKRNRYYDEDEDEE

SEQ ID NO: 21 - protein, 606 aa, Homo sapiens
MSTASAASSSSSSSAGEMIEAPSQVLNFEEIDYKEIEVEEVVGRGAFGVVCKAKWRAKDVAIKQIESESERKAFIVELRQLSRVNHPNIVKLYGACLNPVCLVMEYAEGGSLYNVLHGAEPLPYYTAAHAMSWCLQCSQGVAYLHSMQPKALIHRDLKPPNLLLVAGGTVLKICDFGTACDIQTHMTNNKGSAAWMAPEVFEGSNYSEKCDVFSWGIILWEVITRRKPFDEIGGPAFRIMWAVHNGTRPPLIKNLPKPIESLMTRCWSKDPSQRPSMEEIVKIMTHLMRYFPGADEPLQYPCQYSDEGQSNSATSTGSFMDIASTNTSNKSDTNMEQVPATNDTIKRLESKLLKNQAKQQSESGRLSLGASRGSSVESLPPTSEGKRMSADMSEIEARIAATTAYSKPKRGHRKTASFGNILDVPEIVISGNGQPRRRSIQDLTVTGTEPGQVSSRSSSPSVRMITTSGPTSEKPTRSHPWTPDDSTDTNGSDNSIPMAYLTLDHQLQPLAPCPNSKESMAVFEQHCKMAQEYMKVQTEIALLLQRKQELVAELDQDEKDQQNTSRLVQEHKKLLDENKSLSTYYQQCKKQLEVIRSQQQKRQGTS

SEQ ID NO: 22 - protein, 522 aa, Homo sapiens
MSLLNCENSCGSSQSESDCCVAMASSCSAVTKDDSVGGTASTGNLSSSFMEEIQGYDVEFDPPLESKYECPICLMALREAVQTPCGHRFCKACIIKSIRDAGHKCPVDNEILLENQLFPDNFAKREILSLMVKCPNEGCLHKMELRHLEDHQAHCEFALMDCPQCQRPFQKFHINIHILKDCPRRQVSCDNCAASMAFEDKEIHDQNCPLANVICEYCNTILIREQMPNHYDLDCPTAPIPCTFSTFGCHEKMQRNHLARHLQENTQSHMRMLAQAVHSLSVIPDSGYISEVRNFQETIHQLEGRLVRQDHQIRELTAKMETQSMYVSELKRTIRTLEDKVAEIEAQQCNGIYIWKIGNFGMHLKCQEEEKPVVIHSPGFYTGKPGYKLCMRLHLQLPTAQRCANYISLFVHTMQGEYDSHLPWPFQGTIRLTILDQSEAPVRQNHEEIMDAKPELLAFQRPTIPRNPKGFGYVTFMHLEALRQRTFIKDDTLLVRCEVSTRFDMGSLRREGFQPRSTDAGV

SEQ ID NO: 23 - protein, 504 aa, Homo sapiens
MAAQRRSLLQSEQQPSWTDDLPLCHLSGVGSASNRSYSADGKGTESHPPEDSWLKFRSENNCFLYGVFNGYDGNRVTNFVAQRLSAELLLGQLNAEHAEADVRRVLLQAFDVVERSFLESIDDALAEKASLQSQLPEGVPQHQLPPQYQKILERLKTLEREISGGAMAVVAVLLNNKLYVANVGTNRALLCKSTVDGLQVTQLNVDHTTENEDELFRLSQLGLDAGKIKQVGIICGQESTRRIGDYKVKYGYTDIDLLSAAKSKPIIAEPEIHGAQPLDGVTGFLVLMSEGLYKALEAAHGPGQANQEIAAMIDTEFAKQTSLDAVAQAVVDRVKRIHSDTFASGGERARFCPRHEDMTLLVRNFGYPLGEMSQPTPSPAPAAGGRVYPVSVPYSSAQSTSKTSVTLSLVMPSQGQMVNGAHSASTLDEATPTLTNQSPTLTLQSTNTHTQSSSSSSDGGLFRSRPAHSLPPGEDGRVEPYVDFAEFYRLWSVDHGEQSVVTAP

SEQ ID NO: 24 - protein, 693 aa, Homo sapiens
MAQGSHQIDFQVLHDLRQKFPEVPEVVVSRCMLQNNNNLDACCAVLSQESTRYLYGEGDLNFSDDSGISGLRNHMTSLNLDLQSQNIYHHGREGSRMNGSRTLTHSISDGQLQGGQSNSELFQQEPQTAPAQVPQGFNVFGMSSSSGASNSAPHLGFHLGSKGTSSLSQQTPRFNPIMVTLAPNIQTGRNTPTSLHIHGVPPPVLNSPQGNSIYIRPYITTPGGTTRQTQQHSGWVSQFNPMNPQQVYQPSQPGPWTTCPASNPLSHTSSQQPNQQGHQTSHVYMPISSPTTSQPPTIHSSGSSQSSAHSQYNIQNISTGPRKNQIEIKLEPPQRNNSSKLRSSGPRTSSTSSSVNSQTLNRNQPTVYIAASPPNTDELMSRSQPKVYISANAATGDEQVMRNQPTLFISTNSGASAASRNMSGQVSMGPAFIHHHPPKSRAIGNNSATSPRVVVTQPNTKYTFKITVSPNKPPAVSPGVVSPTFELTNLLNHPDHYVETENIQHLTDPTLAHVDRISETRKLSMGSDDAAYTQALLVHQKARMERLQRELEIQKKKLDKLKSEVNEMENNLTRRRLKRSNSISQIPSLEEMQQLRSCNRQLQIDIDCLTKEIDLFQARGPHFNPSAIHNFYDNIGFVGPVPPKPKDQRSIIKTPKTQDTEDDEGAQWNCTACTFLNHPALIRCEQCEMPRHF

SEQ ID NO: 25 - protein, 712 aa, Homo sapiens
MAQSSPQLDIQVLHDLRQRFPEIPEGVVSQCMLQNNNNLEACCRALSQESSKYLYMEYHSPDDNRMNRNRLLHINLGIHSPSSYHPGDGAQLNGGRTLVHSSSDGHIDPQHAAGKQLICLVQEPHSAPAVVAATPNYNPFFMNEQNRSAATPPSQPPQQPSSMQTGMNPSAMQGPSPPPPPPSYMHIPRYSTNPITVTVSQNLPSGQTVPRALQILPQIPSNLYGSPGSIYIRQTSQSSSGRQTPQSTPWQSSPQGPVPHYSQRPLPVYPHQQNYQPSQYSPKQQQIPQSAYHSPPPSQCPSPFSSPQHQVQPSQLGHIFMPPSPSTTPPHPYQQGPPSYQKQGSHSVAYLPYTASSLSKGSMKKIEITVEPSQRPGTAINRSPSPISNQPSPRNQHSLYTATTPPSSSPSRGISSQPKPPFSVNPVYITYTQPTGPSCTPSPSPRVIPNPTTVFKITVGRATTENLLNLVDQEERSAAPEPIQPISVIPGSGGEKGSHKYQRSSSSGSDDYAYTQALLLHQRARMERLAKQLKLEKEELERLKSEVNGMEHDLMQRRLRRVSCTTAIPTPEEMTRLRSMNRQLQINVDCTLKEVDLLQSRGNFDPKAMNNFYDNIEPGPVVPPKPSKKDSSDPCTIERKARRISVTSKVQADIHDTQAAAADEHRTGSTQSPRTQPRDEDYEGAPWNCDSCTFLNHPALNRCEQCEMPRYT

SEQ ID NO: 26 - protein, 419 aa, Homo sapiens
MAASSLEQKLSRLEAKLKQENREARRRIDLNLDISPQRPRPTLQLPLANDGGSRSPSSESSPQHPTPPARPRHMLGLPSTLFTPRSMESIEIDQKLQEIMKQTGYLTIGGQRYQAEINDLENLGEMGSGTCGQVWKMRFRKTGHVIAVKQMRRSGNKEENKRILMDLDVVLKSHDCPYIVQCFGTFITNTDVFIAMELMGTCAEKLKKRMQGPIPERILGKMTVAIVKALYYLKEKHGVIHRDVKPSNILLDERGQIKLCDFGISGRLVDSKAKTRSAGCAAYMAPERIDPPDPTKPDYDIRADVWSLGISLVELATGQFPYKNCKTDFEVLTKVLQEEPPLLPGHMGFSGDFQSFVKDCLTKDHRKRPKYNKLLEHSFIKRYETLEVDVASWFKDVMAKTESPRTSGVLSQPHLPFFR

SEQ ID NO: 27 - protein, 745 aa, Homo sapiens
MERPPGLRPGAGGPWEMRERLGTGGFGNVCLYQHRELDLKIAIKSCRLELSTKNRERWCHEIQIMKKLNHANVVKACDVPEELNILIHDVPLLAMEYCSGGDLRKLLNKPENCCGLKESQILSLLSDIGSGIRYLHENKIIHRDLKPENIVLQDVGGKIIHKIIDLGYAKDVDQGSLCTSFVGTLQYLAPELFENKPYTATVDYWSFGTMVFECIAGYRPFLHHLQPFTWHEKIKKKDPKCIFACEEMSGEVRFSSHLPQPNSLCSLVVEPMENWLQLMLNWDPQQRGGPVDLTLKQPRCFVLMDHILNLKIVHILNMTSAKIISFLLPPDESLHSLQSRIERETGINTGSQELLSETGISLDPRKPASQCVLDGVRGCDSYMVYLFDKSKTVYEGPFASRSLSDCVNYIVQDSKIQLPIIQLRKVWAEAVHYVSGLKEDYSRLFQGQRAAMLSLLRYNANLTKMKNTLISASQQLKAKLEFFHKSIQLDLERYSEQMTYGISSEKMLKAWKEMEEKAIHYAEVGVIGYLEDQIMSLHAEIMELQKSPYGRRQGDLMESLEQRAIDLYKQLKHRPSDHSYSDSTEMVKIIVHTVQSQDRVLKELFGHLSKLLGCKQKIIDLLPKVEVALSNIKEADNTVMFMQGKRQKEIWHLLKIACTQSSARSLVGSSLEGAVTPQTSAWLPPTSAEHDHSLSCVVTPQDGETSAQMIEENLNCLGHLSTIIHEANEEQGNSMMNLDWSWLTE

SEQ ID NO: 28 - protein, 756 aa, Homo sapiens
MSWSPSLTTQTCGAWEMKERLGTGGFGNVIRWHNQETGEQIAIKQCRQELSPRNRERWCLEIQIMRRLTHPNVVAARDVPEGMQNLAPNDLPLLAMEYCQGGDLRKYLNQFENCCGLREGAILTLLSDIASALRYLHENRIIHRDLKPENIVLQQGEQRLIHKIIDLGYAKELDQGSLCTSFVGTLQYLAPELLEQQKYTVTVDYWSFGTLAFECITGFRPFLPNWQPVQWHSKVRQKSEVDIVVSEDLNGTVKFSSSLPYPNNLNSVLAERLEKWLQLMLMWHPRQRGTDPTYGPNGCFKALDDILNLKLVHILNMVTGTIHTYPVTEDESLQSLKARIQQDTGIPEEDQELLQEAGLALIPDKPATQCISDGKLNEGHTLDMDLVFLFDNSKITYETQISPRPQPESVSCILQEPKRNLAFFQLRKVWGQVWHSIQTLKEDCNRLQQGQRAAMMNLLRNNSCLSKMKNSMASMSQQLKAKLDFFKTSIQIDLEKYSEQTEFGITSDKLLLAWREMEQAVELCGRENEVKLLVERMMALQTDIVDLQRSPMGRKQGGTLDDLEEQARELYRRLREKPRDQRTEGDSQEMVRLLLQAIQSFEKKVRVIYTQLSKTVVCKQKALELLPKVEEVVSLMNEDEKTVVRLQEKRQKELWNLLKIACSKVRGPVSGSPDSMNASRLSQPGQLMSQPSTASNSLPEPAKKSEELVAEAHNLCTLLENAIQDTVREQDQSFTALDWSWLQTEEEEHSCLEQAS

SEQ ID NO: 29 - protein, 419 aa, Homo sapiens
MNRHLWKSQLCEMVQPSGGPAADQDVLGEESPLGKPAMLHLPSEQGAPETLQRCLEENQELRDAIRQSNQILRERCEELLHFQASQREEKEFLMCKFQEARKLVERLGLEKLDLKRQKEQALREVEHLKRCQQQMAEDKASVKAQVTSLLGELQESQSRLEAATKECQALEGRARAASEQARQLESEREALQQQHSVQVDQLRMQGQSVEAALRMERQAASEEKRKLAQLQVAYHQLFQEYDNHIKSSVVGSERKRGMQLEDLKQQLQQAEEALVAKQEVIDKLKEEAEQHKIVMETVPVLKAQADIYKADFQAERQAREKLAEKKELLQEQLEQLQREYSKLKASCQESARIEDMRKRHVEVSQAPLPPAPAYLSSPLALPSQRRSPPEEPPDFCCPKCQYQAPDMDTLQIHVMECIE

SEQ ID NO: 30 - protein, 317 aa, Homo sapiens
MFQAAERPQEWAMEGPRDGLKKERLLDDRHDSGLDSMKDEEYEQMVKELQEIRLEPQEVPRGSEPWKQQLTEDGDSFLHLAIIHEEKALTMEVIRQVKGDLAFLNFQNNLQQTPLHLAVITNQPEIAEALLGAGCDPELRDFRGNTPLHLACEQGCLASVGVLTQSCTTPHLHSILKATNYNGHTCLHLASIHGYLGIVELLVSLGADVNAQEPCNGRTALHLAVDLQNPDLVSLLLKCGADVNRVTYQGYSPYQLTWGRPSTRIQQQLGQLTLENLQMLPESEDEESYDTESEFTEFTEDELPYDDCVFGGQRLTL

SEQ ID NO: 31 - protein, 968 aa, Homo sapiens
MAEDDPYLGRPEQMFHLDPSLTHTIFNPEVFQPQMALPTDGPYLQILEQPKQRGFRFRYVCEGPSHGGLPGASSEKNKKSYPQVKICNYVGPAKVIVQLVTNGKNIHLHAHSLVGKHCEDGICTVTAGPKDMVVGFANLGILHVTKKKVFETLEARMTEACIRGYNPGLLVHPDLAYLQAEGGGDRQLGDREKELIRQAALQQTKEMDLSVVRLMFTAFLPDSTGSFTRRLEPVVSDAIYDSKAPNASNLKIVRMDRTAGCVTGGEEIYLLCDKVQKDDIQIRFYEEEENGGVWEGFGDFSPTDVHRQFAIVFKTPKYKDINITKPASVFVQLRRKSDLETSEPKPFLYYPEIKDKEEVQRKRQKLMPNFSDSFGGGSGAGAGGGGMFGSGGGGGGTGSTGPGYSFPHYGFPTYGGITFHPGTTKSNAGMKHGTMDTESKKDPEGCDKSDDKNTVNLFGKVIETTEQDQEPSEATVGNGEVTLTYATGTKEESAGVQDNLFLEKAMQLAKRHANALFDYAVTGDVKMLLAVQRHLTAVQDENGDSVLHLAIIHLHSQLVRDLLEVTSGLISDDIINMRNDLYQTPLHLAVITKQEDVVEDLLRAGADLSLLDRLGNSVLHLAAKEGHDKVLSILLKHKKAALLLDHPNGDGLNAIHLAMMSNSLPCLLLLVAAGADVNAQEQKSGRTALHLAVEHDNISLAGCLLLEGDAHVDSTTYDGTTPLHIAAGRGSTRLAALLKAAGADPLVENFEPLYDLDDSWENAGEDEGVVPGTTPLDMATSWQVFDILNGKPYEPEFTSDDLLAQGDMKQLAEDVKLQLYKLLEIPDPDKNWATLAQKLGLGILNNAFRLSPAPSKTLMDNYEVSGGTVRELVEALRQMGYTEAIEVIQAASSPVKTTSQAHSLPLSPASTRQQIDELRDSDSVCDSGVETSFRKLSFTESLTSGASLLTLNKMPHDYGQEGPLEGKI

SEQ ID NO: 32 - protein, 551 aa, Homo sapiens
MDELFPLIFPAEPAQASGPYVEIIEQPKQRGMRFRYKCEGRSAGSIPGERSTDTTKTHPTIKINGYTGPGTVRISLVTKDPPHRPHPHELVGKDCRDGFYEAELCPDRCIHSFQNLGIQCVKKRDLEQAISQRIQTNNNPFQVPIEEQRGDYDLNAVRLCFQVTVRDPSGRPLRLPPVLSHPIFDNRAPNTAELKICRVNRNSGSCLGGDEIFLLCDKVQKEDIEVYFTGPGWEARGSFSQADVHRQVAIVFRTPPYADPSLQAPVRVSMQLRRPSDRELSEPMEFQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 33 - protein, 619 aa, Homo sapiens
MASGAYNPYIEIIEQPRQRGMRFRYKCEGRSAGSIPGEHSTDNNRTYPSIQIMNYYGKGKVRITLVTKNDPYKPHPHDLVGKDCRDGYYEAEFGQERRPLFFQNLGIRCVKKKEVKEAIITRIKAGINPFNVPEKQLNDIEDCDLNVVRLCFQVFLPDEHGNLTTALPPVVSNPIYDNRAPNTAELRICRVNKNCGSVRGGDEIFLLCDKVQKDDIEVRFVLNDWEAKGIFSQADVHRQVAIVFKTPPYCKAITEPVTVKMQLRRPSDQEVSESMDFRYLPDEKDTYGNKAKKQKTTLLFQKLCQDHVETGFRHVDQDGLELLTSGDPPTLASQSAGITVNFPERPRPGLLGSIGEGRYFKKEPNLFSHDAVVREMPTGVSSQAESYYPSPGPISSGLSHHASMAPLPSSSWSSVAHPTPRSGNTNPLSSFSTRTLPSNSQGIPPFLRIPVGNDLNASNACIYNNADDIVGMEASSMPSADLYGISDPNMLSNCSVNMMTTSSDSMGETDNPRLLSMNLENPSCNSVLDPRDLRQLHQMSSSSMSAGANSNTTVFVSQSDAFEGSDFSCADNSMINESGPSNSTNPNSHGFVQDSQYSGIGSMQNEQLSDSFPYEFFQV

SEQ ID NO: 34 - protein, 427 aa, Homo sapiens
MSRSKRDNNFYSVEIGDSTFTVLKRYQNLKPIGSGAQGIVCAAYDAILERNVAIKKLSRPFQNQTHAKRAYRELVLMKCVNHKNIIGLLNVFTPQKSLEEFQDVYIVMELMDANLCQVIQMELDHERMSYLLYQMLCGIKHLHSAGIIHRDLKPSNIVVKSDCTLKILDFGLARTAGTSFMMTPYVVTRYYRAPEVILGMGYKENVDLWSVGCIMGEMVCHKILFPGRDYIDQWNKVIEQLGTPCPEFMKKLQPTVRTYVENRPKYAGYSFEKLFPDVLFPADSEHNKLKASQARDLLSKMLVIDASKRISVDEALQHPYINVWYDPSEAEAPPPKIPDKQLDEREHTIEEWKELIYKEVMDLEERTKNGVIRGQPSPLGAAVINGSQHPSSSSSVNDVSSMSTDPTLASDTDSSLEAAAGPLGCCR

SEQ ID NO: 35 - protein, 424 aa, Homo sapiens
MSDSKCDSQFYSVQVADSTFTVLKRYQQLKPIGSGAQGIVCAAFDTVLGINVAVKKLSRPFQNQTHAKRAYRELVLLKCVNHKNIISLLNVFTPQKTLEEFQDVYLVMELMDANLCQVIHMELDHERMSYLLYQMLCGIKHLHSAGIIHRDLKPSNIVVKSDCTLKILDFGLARTACTNFMMTPYVVTRYYRAPEVILGMGYKENVDIWSVGCIMGELVKGCVIFQGTDHIDQWNKVIEQLGTPSAEFMKKLQPTVRNYVENRPKYPGIKFEELFPDWIFPSESERDKIKTSQARDLLSKMLVIDPDKRISVDEALRHPYITVWYDPAEAEAPPPQIYDAQLEEREHAIEEWKELIYKEVMDWEERSKNGVVKDQPSDAAVSSNATPSQSSSINDISSMSTEQTLASDTDSSLDASTGPLEGCR

SEQ ID NO: 36 - protein, 464 aa, Homo sapiens
MSLHFLYYCSEPTLDVKIAFCQGFDKQVDVSYIAKHYNMSKSKVDNQFYSVEVGDSTFTVLKRYQNLKPIGSGAQGIVCAAYDAVLDRNVAIKKLSRPFQNQTHAKRAYRELVLMKCVNHKNIISLLNVFTPQKTLEEFQDVYLVMELMDANLCQVIQMELDHERMSYLLYQMLCGIKHLHSAGIIHRDLKPSNIVVKSDCTLKILDFGLARTAGTSFMMTPYVVTRYYRAPEVILGMGYKENVDIWSVGCIMGEMVRHKILFPGRDYIDQWNKVIEQLGTPCPEFMKKLQPTVRNYVENRPKYAGLTFPKLFPDSLFPADSEHNKLKASQARDLLSKMLVIDPAKRISVDDALQHPYINVWYDPAEVEAPPPQIYDKQLDEREHTIEEWKELIYKEVMNSEEKTKNGVVKGQPSPSGAAVNSSESLPPSSSVNDISSMSTDQTLASDTDSSLEASAGPLGCCR

(19) United States
(12) Patent Application Publication
(10) Pub. No.: US 2026/0049085 A1
(43) Pub. Date: Feb. 19, 2026

(54) CYCLIC COMPOUNDS AND METHODS OF USING SAME

(71) Applicant: SCHRÖDINGER, INC., New York, NY (US)

(72) Inventors: Shelby ELLERY, New York, NY (US); Shulu FENG, New York, NY (US); Jiaye GUO, New York, NY (US); Goran KRILOV, New York, NY (US); Ana NEGRI, New York, NY (US); Zhe NIE, New York, NY (US); Robert PELLETIER, New York, NY (US); Andrew PLAZCEK, New York, NY (US); Mats SVENSSON, New York, NY (US); Michael TRZOSS, New York, NY (US)

(73) Assignee: SCHRÖDINGER, INC., New York, NY (US)

(21) Appl. No.: 18/996,098

(22) PCT Filed: Jul. 21, 2023
(86) PCT No.: PCT/US2023/070683

§ 371 Date: Jan. 17, 2025

Related U.S. Application Data
(60) Provisional application No. 63/391,548, filed on Jul. 22, 2022.

(51) Int. Cl. C07D 487/04; A61K 31/519

(52) U.S. Cl. CPC C07D 487/04; A61K 31/519

(57) ABSTRACT

The present application relates to compounds of Formula (I), as defined herein, and pharmaceutically acceptable salts thereof. The present application also describes pharmaceutical composition comprising a compound of Formula (I), and pharmaceutically acceptable salts thereof, and methods of using the compounds and compositions for treating diseases, such as cancer, autoimmune disorders, and inflammatory disorders.

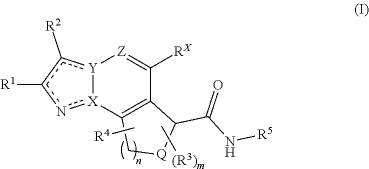

### CROSS-REFERENCE TO RELATED APPLICATIONS

This application claims the benefit of U.S. Provisional Application No. 63/391,548, filed Jul. 22, 2022, which is incorporated by reference herein in its entirety.

### TECHNICAL FIELD

This present application relates to tricyclic that are useful for treating proliferative disorders such as cancer, as well as autoimmune and inflammatory disorders.

### BACKGROUND

MALT1 (mucosa-associated lymphoid tissue lymphoma translocation protein 1) is an intracellular protein involved in lymphocyte proliferation via upstream signaling of NF-κB to control lymphocyte activation, survival, proliferation, and differentiation. Together with a CARMA or CARD scaffold protein, (e.g., CARD11 (caspase recruitment domain family member 11, also known as CARMA1), CARD14 (caspase recruitment domain family member 14, also known as CARMA2), CARD10 (caspase recruitment domain family member 10, also known as CARMA3), or CARD9 (caspase recruitment domain family member 9)) and BCL10 (B-cell CLL/Lymphoma 10), MALT1 is one of the three subunits of the CBM complex which is formed upon cell-surface antigen receptor activation. See Jaworski et al., Cell Mol Life Science 2016, 73, 459-473, and Juilland and Thome. Frontiers in Immunology 2018, 9, 1927. MALT1 is known to mediate NF-κB signaling by at least two mechanisms: firstly, MALT1 functions as a scaffold protein, recruiting NF-κB signaling proteins such as TRAF6, TABs (e.g., TAB1, TAB2, TAB3), TAK1 and NEMO-IKKβ; and secondly, as a cysteine protease, it cleaves and deactivates negative regulators of NF-κB signaling, such as RelB, A20, or CYLD. See Rosebeck et al., Science, 2011, 331, 468-472.

The protease activity of MALT1 has emerged as a potential therapeutic target, particularly where NF-κB and related pathways are believed to play a significant role. Activated B cell-like diffuse large B cell lymphomas (ABC-DLBCLs) are aggressive lymphomas that are often characterized by NF-κB hyperactivation, and it has been shown that MALT1 protease inhibition can dramatically inhibit growth and promote apoptosis of the highly aggressive ABC type DLBCLs. See Ferch U, et al., J Exp Med 2009, 206, 2313-2320; see also, Hailfinger S, et al., Proc Natl Acad Sci USA 2009, 106, 19946-19951. Known peptide substrates of MALT1, or fusion protein API2-MALT1, include A20, CYLD, BCL10, RelB, regnase-1, roquin-1, NIK, and LIMA la. See Rebeaud et al., Nat Immunol 2008, 9, 272-281; see also, Coornaert et al., Nat Immunol 20008, 9, 263-271; Staal et al., EMBO J 2011, 30, 1742-1752; Hailfinger et al., PNAS 2011, 108, 14596-14601; Jeltsch et al., Nat Immunol 2014, 15, 1079-1089; Uehata et al., Cell 2013, 153, 1036-1049; Nie et al., Nat Commun 2015, 6, 5908; and Baens et al., PLoS ONE 2014, 9, e103774. One general profile of MALT1 substrates is described in Kasperkiewicz, et al. Scientific Reports 8.1 (2018): 1-10.

Additionally, several chromosomal translocations that lead to the generation of constitutively active MALT1 have been identified in ABC-DLBCLs and the identification of the MALT1 fusion protein API2-MALT1/IgH-MALT1 that leads to NF-κB activation independent of upstream stimulation further highlights the importance of this protein in cancer and various diseases. See Farinha et al., J Clinical Oncology 2005, 23, 6370-6378. Further, MALT1 has been shown to be involved in several different types of cancers, for example hematological malignancies such as mantle cell lymphoma, chronic lymphocytic leukemia (CLL) and solid tumors such as lung adenocarcinoma, breast cancer, pancreatic cancer, and glioblastoma. See Jiang et al., Cancer Research 2011, 71. 2183-2192; see also, Pan et al., Mol Cancer Res 2016, 14, 93-102, Penas et al., Blood 2010, 115, 2214-2219, and J Cell Mol Med. 2020 July; 24(13):7550-7562. MALT1, as an immunomodulatory protein, is also involved in innate and adaptive immunity and may have effects on several inflammatory disorders, e.g., psoriasis, multiple sclerosis, rheumatoid arthritis, Sjogren's syndrome, ulcerative colitis, and different types of allergic disorders resulting from chronic inflammation. See Afofina et al., FEBS Journal 2015, DOI: 10.1 111/febs. 13325; see also Lowes et al., Ann Review Immunology 2014, 32, 227-255; Jabara et al., J Allergy Clin Immunology 2013, 132, 151-158; Streubel et al., Clin Cancer Research 2004, 10, 476-480; and Liu et al., Oncotarget 2016, 1-14. Recently, findings also suggest the importance of MALT1 in the control of regulatory T cell (Treg) function and homeostasis. Studies are ongoing to confirm the potential of MALT1 inhibitors for the treatment of patients with solid tumors alone or in combination with immune-checkpoint mechanisms. However, no MALT1 inhibitors are currently approved for therapeutic use.

### SUMMARY

Accordingly, provided herein is a compound of the Formula (I):

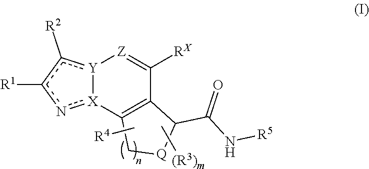

or a pharmaceutically acceptable salt thereof, wherein X, Y, Z, Q, n, R^{X}, R¹, R², R³, m, R⁴ and R⁵ are as defined herein.

Also provided herein is a pharmaceutical composition comprising a compound of Formula (I), or a pharmaceutically acceptable salt thereof, and at least one pharmaceutically acceptable excipient.

Also provided are methods for treating a CBM complex pathway-associated cancer in a subject in need thereof, comprising administering to the subject an effective amount of a compound of Formula (I), or a pharmaceutically acceptable salt thereof, or a pharmaceutical composition as described herein.

Also provided are methods for treating a cancer in a subject in need thereof, comprising:
(a) identifying the cancer as being a CBM complex pathway-associated cancer; and
(b) administering to the subject an effective amount of a compound of Formula (I), or a pharmaceutically acceptable salt thereof, or a pharmaceutical composition as described herein.

Also provided are methods for treating a cancer in a subject in need thereof, comprising:
administering an effective amount of a compound of Formula (I), or a pharmaceutically acceptable salt thereof, or a pharmaceutical composition as described herein, to a subject identified as having a CBM complex pathway-associated cancer.

Also provided are methods for treating a MALT1-associated cancer in a subject, comprising administering to a subject identified or diagnosed as having a MALT1-associated cancer an effective amount of a compound of Formula (I), or a pharmaceutically acceptable salt thereof, or a pharmaceutical composition as described herein.

Also provided are methods for treating cancer in a subject in need thereof, comprising:
(a) determining that the cancer is associated with a dysregulation of a MALT1 gene, a MALT1 protease, or expression or activity or level of any of the same; and
(b) administering to the subject an effective amount of a compound of Formula (I), or a pharmaceutically acceptable salt thereof, or a pharmaceutical composition as described herein.

Also provided are methods for inhibiting metastasis in a subject having a cancer in need of such treatment, comprising administering to the subject an effective amount of a compound of Formula (I), or a pharmaceutically acceptable salt thereof, or a pharmaceutical composition as described herein.

Also provided are methods for treating an autoimmune disorder in a subject in need thereof, comprising administering to the subject an effective amount of a compound of Formula (I), or a pharmaceutically acceptable salt thereof, or a pharmaceutical composition as described herein.

Also provided are methods for treating a CBM complex pathway-associated disease or disorder in a subject in need thereof, comprising administering to the subject an effective amount of a compound of Formula (I), or a pharmaceutically acceptable salt thereof, or a pharmaceutical composition as described herein.

Also provided are methods for treating a disease or disorder in a subject in need thereof, comprising:
(a) identifying the disease or disorder as being a CBM complex pathway-associated disease or disorder; and
(b) administering to the subject an effective amount of a compound of Formula (I), or a pharmaceutically acceptable salt thereof, or a pharmaceutical composition as described herein.

Also provided are methods for treating a disease or disorder in a subject in need thereof, comprising:
administering to the subject an effective amount of a compound of Formula (I), or a pharmaceutically acceptable salt thereof, or a pharmaceutical composition as described herein, to a subject identified as having a CBM complex pathway-associated disease or disorder.

Also provided are methods for treating a MALT1-associated autoimmune disorder in a subject, comprising administering to a subject identified or diagnosed as having a MALT1-associated autoimmune disorder an effective amount of a compound of Formula (I), or a pharmaceutically acceptable salt thereof, or a pharmaceutical composition as described herein.

Also provided are methods for treating a MALT1-associated autoimmune disorder in a subject, comprising administering to a subject identified or diagnosed as having a MALT1-associated autoimmune disorder an effective amount of a compound of Formula (I), or a pharmaceutically acceptable salt thereof, or a pharmaceutical composition as described herein.

Also provided are methods for treating an autoimmune disorder in a subject in need thereof, comprising:
(a) determining that the autoimmune disorder is associated with a dysregulation of a MALT1 gene, a MALT1 protease, or expression or activity or level of any of the same; and
(b) administering to the subject an effective amount of a compound of Formula (I), or a pharmaceutically acceptable salt thereof, or a pharmaceutical composition as described herein.

Also provided are methods for treating a MALT1-associated autoimmune disorder in a subject, comprising administering an effective amount of a compound of Formula (I), or a pharmaceutically acceptable salt thereof, or a pharmaceutical composition as described herein, to a subject determined to have a MALT1-associated autoimmune disorder.

Also provided are methods for treating an inflammatory disorder in a subject in need thereof, comprising administering to the subject an effective amount of a compound of Formula (I), or a pharmaceutically acceptable salt thereof, or a pharmaceutical composition as described herein.

Also provided are methods for treating a MALT1-associated inflammatory disorder in a subject, comprising administering to a subject identified or diagnosed as having a MALT1-associated inflammatory disorder an effective amount of a compound of Formula (I), or a pharmaceutically acceptable salt thereof, or a pharmaceutical composition as described herein.

Also provided are methods for treating a MALT1-associated inflammatory disorder in a subject, comprising administering to a subject identified or diagnosed as having a MALT1-associated inflammatory disorder an effective amount of a compound of Formula (I), or a pharmaceutically acceptable salt thereof, or a pharmaceutical composition as described herein.

Also provided are methods for treating an inflammatory disorder in a subject in need thereof, comprising:
(a) detensioning that the inflammatory disorder is associated with a dysregulation of a MALT1 gene, a MALT1 protease, or expression or activity or level of any of the same; and
(b) administering to the subject an effective amount of a compound of Formula (I), or a pharmaceutically acceptable salt thereof, or a pharmaceutical composition as described herein.

Also provided are methods for treating a MALT1-associated inflammatory disorder in a subject, comprising administering an effective amount of a compound of Formula (I), or a pharmaceutically acceptable salt thereof, or a pharmaceutical composition as described herein, to a subject determined to have a MALT1-associated inflammatory disorder.

Also provided are methods for inhibiting CBM complex pathway activity in a mammalian cell, comprising contacting the mammalian cell with a compound of Formula (I), or a pharmaceutically acceptable salt thereof.

Also provided are methods for inhibiting MALT1 protease activity in a mammalian cell, comprising contacting the mammalian cell with a compound of Formula (I), or a pharmaceutically acceptable salt thereof.

Also provided are the use of compounds of Formula (I), or pharmaceutically acceptable salts thereof, for treating a CBM complex pathway-associated disease or disorder.

Also provided are compounds of Formula (T), or pharmaceutically acceptable salts thereof, for use in the manufacture of a medicament for the treatment of a CBM complex pathway-associated disease or disorder.

Also provided are methods of treating an individual with a MALT1-associated cancer that include administering a compound of Formula (I), or a pharmaceutically acceptable salt thereof, before, during, or after administration of other anticancer drug(s) (e.g., a first MALT1 inhibitor or another MALT1 inhibitor).

Also provided herein is a process for preparing a compound of Formula (I), or a pharmaceutically acceptable salt thereof.

Also provided herein is a compound of Formula (I), or a pharmaceutically acceptable salt thereof obtained by a process of preparing the compound as defined herein.

Unless otherwise defined, all technical and scientific terms used herein have the same meaning as commonly understood by one of ordinary skill in the art to which this disclosure belongs. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entireties. In case of conflict, the present specification, including definitions, will control.

Other features and advantages of the disclosure will be apparent from the following detailed description and from the claims.

### DETAILED DESCRIPTION

The term “compound,” as used herein is meant to include all stereoisomers, geometric isomers, tautomers, and isotopically enriched variants of the structures depicted. Compounds herein identified by name or structure as one particular tautomeric form are intended to include other tautomeric forms unless otherwise specified.

The term “tautomer,” as used herein refers to compounds whose structures differ markedly in arrangement of atoms, but which exist in easy and rapid equilibrium, and it is to be understood that compounds provided herein may be depicted as different tautomers, and when compounds have tautomeric forms, all tautomeric forms are intended to be within the scope of the disclosure, and the naming of the compounds does not exclude any tautomer. The following is an example of included tautomeric forms:

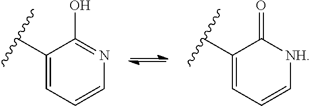 [SMILES: *c1cccnc1O ; *c1ccc[nH]c1=O]

It will be appreciated that certain compounds provided herein may contain one or more centers of asymmetry and may therefore be prepared and isolated in a mixture of isomers such as a racemic mixture, or in an enantiomerically pure form.

The term “halo or halogen” refers to one of the halogens, group 17 of the periodic table. In particular, the term refers to fluorine, chlorine, bromine and iodine. Preferably, the term refers to fluorine or chlorine.

The term “C1-C6 alkyl” refers to a linear or branched hydrocarbon chain containing 1, 2, 3, 4, 5 or 6 carbon atoms, for example methyl, ethyl, n-propyl, iso-propyl, n-butyl, sec-butyl, tert-butyl, n-pentyl and n-hexyl. Similarly, a C1-C3 alkyl group is linear or branched hydrocarbon chain containing 1, 2, or 3 carbon atoms.

The term “C1-C6 haloalkyl” refers to a hydrocarbon chain substituted with at least one halogen atom independently chosen at each occurrence, for example fluorine, chlorine, bromine and iodine. The halogen atom may be present at any position on the hydrocarbon chain. Similarly, a C1-C3 haloalkyl group is linear or branched hydrocarbon chain containing 1, 2, or 3 carbon atoms substituted with at least one halogen atom. For example, C1-C3 haloalkyl may refer to chloromethyl, fluoromethyl, difluoromethyl, trifluoromethyl, chloroethyl e.g. 1-chloroethyl and 2-chloroethyl, trichloroethyl e.g. 1,2,2-trichloroethyl, 2,2,2-trichloroethyl, fluoroethyl e.g. 1-fluoromethyl and 2-fluoroethyl, trifluoroethyl e.g. 1,2,2-trifluoroethyl and 2,2,2-trifluoroethyl, chloropropyl, trichloropropyl, fluoropropyl, trifluoropropyl.

The term “C1-C3 alkoxy” refers to a C1-C3 alkyl group which is attached to a molecule via oxygen. This includes moieties where the alkyl part may be linear or branched, such as methoxy, ethoxy, n-propoxy, and iso-propoxy.

The term “C1-C3 haloalkoxy” refers to a C1-C3 alkyl group which is attached to a molecule via oxygen and where at least one hydrogen atom of the alkyl group is replaced with a halogen. This includes moieties where the alkyl part may be linear or branched, such as fluoromethoxy, difluoromethoxy, trifluoromethoxy, 2,2,2-trifluoroethoxy, or trifluoropropoxy.

A [symbol] represents a single or double bond, valence permitting. For example,

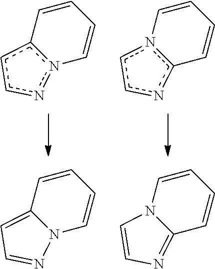

As used herein, the term “cyano” refers to a —CN radical.

As used herein, the term “hydroxyl” refers to an —OH radical.

As used herein, the term “amino” refers to an —NH₂ group.

As used herein, the term “aryl” refers to a 6-10 all carbon mono- or bicyclic group wherein at least one ring in the system is aromatic. Non-limiting examples of aryl groups include phenyl, naphthyl, tetrahydronaphthyl. In bicyclic ring systems where only one ring is aromatic, the non-aromatic ring can be a cycloalkyl group, as defined herein.

As used herein, the term “heteroaryl” refers to a 5-10 membered, or 5-9 membered, mono- or bicyclic group wherein at least one ring in the system is aromatic; wherein one or more carbon atoms in at least one ring in the system is/are replaced with an heteroatom independently selected from N, O, and S. Heteroaryl groups include rings where one or more atoms are oxidized (e.g., carbon, nitrogen, and sulfur), such as a pyridone moiety. Non-limiting examples of heteroaryl groups include pyridine, pyrimidine, pyridazine, pyrimidinone, pyrrole, pyrazole, imidazole, triazole, thiazole, and indole. In bicyclic ring systems where only one ring is aromatic, the non-aromatic ring can be a cycloalkyl or heterocyclyl group, as defined herein.

As used herein, the term “cycloalkyl” refers to a saturated or partially unsaturated 3-10 or 3-7 mono- or bicyclic hydrocarbon group, or a 3-6 membered monocyclic hydrocarbon group; wherein bicyclic systems include fused, spiro (optionally referred to as “spirocycloalkyl” groups), and bridged ring systems. Non-limiting examples of cycloalkyl groups include cyclopropyl, cyclohexyl, spiro[2.3]hexyl, and bicyclo[1.1.1]pentyl.

The term “heterocyclyl” refers to a saturated or partially unsaturated 3-12 membered, 3-8 membered, or 4-6 membered hydrocarbon monocyclic or bicyclic ring system, that is not aromatic, having at least one heteroatom within the ring selected from N, O and S. Bicyclic heterocyclyl groups include fused, spiro (optionally referred to as “spiroheterocyclyl” groups), and bridged ring systems. The heterocyclyl ring system may include oxo substitution at one or more C, N, or S ring members. The heterocyclyl group may be denoted as, for example, a “5-10 membered heterocyclyl group,” which is a ring system containing 5, 6, 7, 8, 9 or 10 atoms at least one being a heteroatom. For example, there may be 1, 2 or 3 heteroatoms, optionally 1 or 2. The heterocyclyl group may be bonded to the rest of the molecule through any carbon atom or through a heteroatom such as nitrogen. Exemplary heterocyclyl groups include, but are not limited to, piperidinyl, piperazinyl, morpholino, tetrahydropyranyl, azetidinyl, oxetanyl, 2-azaspiro[3.3]heptanyl, pyrrolidin-2-one, sulfolane, isothiazoline S,S-dioxide, and decahydronaphthalenyl.

As used herein, the term “oxo” refers to an “═O” group attached to a carbon atom.

As used herein, the symbol [symbol] depicts the point of attachment of an atom or moiety to the indicated atom or group in the remainder of the molecule.

It is to be understood that the ring in compounds of Formula (I) comprising atoms X, Y and Z does not contain more than two adjacent nitrogen atoms.

The compounds of Formula (I) include pharmaceutically acceptable salts thereof. In addition, the compounds of Formula (I) also include other salts of such compounds which are not necessarily pharmaceutically acceptable salts, and which may be useful as intermediates for preparing and/or purifying compounds of Formula (I) and/or for separating enantiomers of compounds of Formula (I). Non-limiting examples of pharmaceutically acceptable salts of compounds of Formula (I) include trifluoroacetic acid and hydrochloride salts.

It will further be appreciated that the compounds of Formula (I) or their salts may be isolated in the form of solvates, and accordingly that any such solvate is included within the scope of the present disclosure. For example, compounds of Formula (I) and salts thereof can exist in unsolvated as well as solvated forms with pharmaceutically acceptable solvents such as water, ethanol, and the like.

In some embodiments, the compounds of Formula (I) include the compounds of Examples 1-196 and stereoisomers and pharmaceutically acceptable salts thereof. In some embodiments, the compounds of Formula (I) include the compounds of Examples 1-196 and pharmaceutically acceptable salts thereof. In some embodiments, the compounds of Examples 1-196 are in the free base form. In some embodiments, the compounds of Examples 1-196 are in the form of pharmaceutically acceptable salts.

The term “pharmaceutically acceptable” indicates that the compound, or salt or composition thereof is compatible chemically and/or toxicologically with the other ingredients comprising a formulation and/or the subject being treated therewith.

Protecting groups can be a temporary substituent which protects a potentially reactive functional group from undesired chemical transformations. The choice of the particular protecting group employed is well within the skill of one of ordinary skill in the art. A number of considerations can determine the choice of protecting group including, but not limited to, the functional group being protected, other functionality present in the molecule, reaction conditions at each step of the synthetic sequence, other protecting groups present in the molecule, functional group tolerance to conditions required to remove the protecting group, and reaction conditions for the thermal decomposition of the compounds provided herein. The field of protecting group chemistry has been reviewed (Greene, T. W. and Wuts, P. G. M. Protective Groups in Organic Synthesis, 2. sup. ed. Wiley: New York, 1991).

A nitrogen protecting group can be any temporary substituent which protects an amine moiety from undesired chemical transformations. Examples of moieties formed when such protecting groups are bonded to an amine include, but are not limited to allylamine, benzylamines (e.g., benzylamine, p-methoxybenzylamine, 2,4-dimethoxybenzylamine, and tritylamine), acetylamide, trichloroacetamide, trifluoroacetamide, pent-4-enamide, phthalimides, carbamates (e.g., methyl carbamate, t-butyl carbamate, benzyl carbamate, allyl carbamates, 2,2,2-trichloroethyl carbamate, and 9-fluorenylmethyl carbamate), imines, and sulfonamides (e.g., benzene sulfonamide, p-toluenesulfonamide, and p-nitrobenzenesulfonamide).

An oxygen protecting group can be any temporary substituent which protects a hydroxyl moiety from undesired chemical transformations. Examples of moieties formed when such protecting groups are bonded to a hydroxyl include, but are not limited to esters (e.g., acetyl, t-butyl carbonyl, and benzoyl), benzyl (e.g., benzyl, p-methoxybenzyl, and 2,4-dimethoxybenzyl, and trityl), carbonates (e.g., methyl carbonate, allyl carbonate, 2,2,2-trichloroethyl carbonate and benzyl carbonate) ketals, and acetals, and ethers.

Compounds provided herein may also contain unnatural proportions of atomic isotopes at one or more of the atoms that constitute such compounds. That is, an atom, in particular when mentioned in relation to a compound according to Formula (I), comprises all isotopes and isotopic mixtures of that atom, either naturally occurring or synthetically produced, either with natural abundance or in an isotopically enriched form. For example, when hydrogen is mentioned, it is understood to refer to ¹H, ²H, ³H or mixtures thereof; when carbon is mentioned, it is understood to refer to ¹¹C, ¹²C, ¹³C, ¹⁴C or mixtures thereof; when nitrogen is mentioned, it is understood to refer to ¹³N, ¹⁴N, ¹⁵N or mixtures thereof; when oxygen is mentioned, it is understood to refer to ¹⁴O, ¹⁵O, ¹⁶O, ¹⁷O, ¹⁸O or mixtures thereof; and when fluoro is mentioned, it is understood to refer to ¹⁸F, ¹⁹F or mixtures thereof; unless expressly noted otherwise. For example, in deuteroalkyl and deuteroalkoxy groups, where one or more hydrogen atoms are specifically replaced with deuterium (²H). As some of the aforementioned isotopes are radioactive, the compounds provided herein therefore also comprise compounds with one or more isotopes of one or more atoms, and mixtures thereof, including radioactive compounds, wherein one or more non-radioactive atoms has been replaced by one of its radioactive enriched isotopes. Radiolabeled compounds are useful as therapeutic agents, e.g., cancer therapeutic agents, research reagents, e.g., assay reagents, and diagnostic agents, e.g., in vivo imaging agents. All isotopic variations of the compounds provided herein, whether radioactive or not, are intended to be encompassed within the scope of the present disclosure.

For illustrative purposes, general methods for preparing the compounds are provided herein as well as key intermediates. For a more detailed description of the individual reaction steps, see the Examples section below. Those skilled in the art will appreciate that other synthetic routes may be used to synthesize the inventive compounds. Although specific starting materials and reagents are depicted in the Schemes and discussed below, other starting materials and reagents can be easily substituted to provide a variety of derivatives and/or reaction conditions. In addition, many of the compounds prepared by the methods described below can be further modified in light of this disclosure using conventional chemistry well known to those skilled in the art.

The ability of selected compounds to act as MALT1 inhibitors may be demonstrated by the biological assays described herein. IC₅₀ values are shown in Table A.

Compounds of Formula (I), or a pharmaceutically acceptable salt thereof, are useful for treating diseases and disorders which can be treated with a MALT1 inhibitor, such as MALT1-associated cancers, including hematological cancers and solid tumors, MALT1-associated autoimmune disorders, and MALT1-associated inflammatory disorders.

As used herein, terms “treat” or “treatment” refer to therapeutic or palliative measures. Beneficial or desired clinical results include, but are not limited to, alleviation, in whole or in part, of symptoms associated with a disease or disorder or condition, diminishment of the extent of disease, stabilized (i.e., not worsening) state of disease, delay or slowing of disease progression, amelioration or palliation of the disease state (e.g., one or more symptoms of the disease), and remission (whether partial or total), whether detectable or undetectable. “Treatment” can also mean prolonging survival as compared to expected survival if not receiving treatment.

As used herein, the term “subject” refers to any animal, including mammals such as humans. In some embodiments, the subject is a human. In some embodiments, the subject has experienced and/or exhibited at least one symptom of the disease or disorder to be treated and/or prevented.

The term “pediatric subject” as used herein refers to a subject under the age of 21 years at the time of diagnosis or treatment. The term “pediatric” can be further be divided into various subpopulations including: neonates (from birth through the first month of life); infants (1 month up to two years of age); children (two years of age up to 12 years of age); and adolescents (12 years of age through 21 years of age (up to, but not including, the twenty-second birthday)). Berhman R E, Kliegman R, Arvin A M, Nelson W E. Nelson Textbook of Pediatrics, 15th Ed. Philadelphia: W.B. Saunders Company, 1996; Rudolph A M, et al. Rudolph's Pediatrics, 21st Ed. New York: McGraw-Hill, 2002; and Avery M D, First LR. Pediatric Medicine, 2nd Ed. Baltimore: Williams & Wilkins; 1994. In some embodiments, a pediatric subject is from birth through the first 28 days of life, from 29 days of age to less than two years of age, from two years of age to less than 12 years of age, or 12 years of age through 21 years of age (up to, but not including, the twenty-second birthday). In some embodiments, a pediatric subject is from birth through the first 28 days of life, from 29 days of age to less than 1 year of age, from one month of age to less than four months of age, from three months of age to less than seven months of age, from six months of age to less than 1 year of age, from 1 year of age to less than 2 years of age, from 2 years of age to less than 3 years of age, from 2 years of age to less than seven years of age, from 3 years of age to less than 5 years of age, from 5 years of age to less than 10 years of age, from 6 years of age to less than 13 years of age, from 10 years of age to less than 15 years of age, or from 15 years of age to less than 22 years of age.

In certain embodiments, compounds of Formula (I), or a pharmaceutically acceptable salt thereof are useful for preventing diseases and disorders as defined herein (for example, autoimmune disorders, inflammatory disorders, and cancer). The term “preventing” as used herein means the prevention of the onset, recurrence or spread, in whole or in part, of the disease or condition as described herein, or a symptom thereof.

The term “regulatory agency” refers to a country's agency for the approval of the medical use of pharmaceutical agents with the country. For example, a non-limiting example of a regulatory agency is the U.S. Food and Drug Administration (FDA).

Signaling through the NF-κB pathway has been implicated in many cancers. See, e.g., Staudt, Cold Spring Harbor Perspectives in Biology 2.6 (2010): a000109, Xia, et al. Cancer Immunol. Res. 2.9 (2014): 823-830, Xia, et al. OncoTargets and Therapy 11 (2018): 2063. NF-κB is a family of transcription factors, including p50, p52, p65 (RelA), RelB, and c-Rel, which can bindto the kB enhancer element as various homo- and heterodimers to induce transcription of a number of genes. Following activation of certain cell-surface receptors (e.g., CD28, BCR, HER1 (also known as EGFR (Epidermal Growth Factor Receptor) and ERBB1), or HER2 (also known as HER2/neu or ERBB2)), a CBM complex is formed via phosphorylation of a CARD or CARMA protein, likely by a protein kinase C (e.g., protein kinase C beta or protein kinase C theta) and recruitment of the BCL10-MALT1 complex. See, e.g., Xia, et al. OncoTargets and Therapy 11 (2018): 2063, Shi, and Sun. Mol. Immunol. 68.2 (2015): 546-557, Xia, et al. Cancer Immunol. Res. 2.9 (2014): 823-830, and Pan, Mol. Cancer Res. 14.1 (2016): 93-102.

As noted hereinabove, the CBM complex can function as a scaffold protein in the activation of the NF-κB pathway. When formed, the CBM complex can activate the IKK complex (e.g., IKKγ (also called NEMO), IKKα, and IKKβ), likely by ubiquitination (e.g., K63-linked ubiquitination) of MALT1, which results in the recruitment, ubiquitination (e.g., K63-linked ubiquitination), and degradation of IKKγ, thereby releasing IKKα and IKKβ to phosphorylate IκB, resulting in the ubiquitination (e.g., K48-linked ubiquitination) and degradation of IκB, releasing the NF-κB transcription factors (typically of the NF-κB1 subtype: p50-RelA and p50-cRel) to the nucleus. This cascade is likely mediated by the ubiquitin ligase TRAF6 (Tumor necrosis factor receptor (TNFR)-associated factor 6). The CBM complex may also affect NF-κB signaling through additional protein complexes, such as TAB1/2-TAK and the linear ubiquitin chain assembly complex (LUBAC). See, e.g., Israel, Cold Spring Harbor Perspectives in Biology 2.3 (2010): a000158, Xia, et al. OncoTargets and Therapy 11 (2018): 2063, Juilland, Front. Immunol. 9 (2018): 1927. MALT1 can also activate the JNK pathway (also called the JNK/AP-1 pathway), though less work has been done to study this area. See, e.g., Juilland, Front. Immunol. 9 (2018): 1927, and Wang, et al., Oncogenesis 6.7 (2017): e365-e365.

In addition, MALT1 has cysteine protease activity. Non-limiting examples of substrates of wild-type MALT1 include BCL10, A20, CYLD, Re1B, Regnase 1, roquin-1, and HOIL1. In addition, the API2-MALT1 (also called cIAP2; amino terminus of inhibitor of apoptosis 2) fusion protein has also been shown to cleave NIK and LIMA1α. BCL10 cleavage by MALT1 is believed to result in BCL10-independent NF-κB activation. By cleaving A20 (TNF Alpha Induced Protein 3), MALT1 can reduce negative regulation of the NF-κB pathway, as A20 is a deubiquitinating enzyme that has been suggested to reduce the ubiquitination of MALT1 and thus recruitment and activation of the IKK complex. CYLD (CYLD Lysine 63 Deubiquitinase) is a deubiquitinating enzyme, and by cleavage of this enzyme, it is believed that MALT1 increases signaling through the NF-κB pathway and/or JNK pathway. Cleavage of RelB typically results in relief of negative regulation of the NF-κB pathway, as RelB forms transcriptionally inactive complexes with RelA and c-Rel. By cleaving HOIL1 (also known as RBCK1), it is believed that negative regulation of the NF-κB is relieved, as HOIL1 is thought to decrease linear ubiquitination. MALT1 can also autoprocess, which promotes signaling through the NF-κB pathway through a mechanism that is not fully understood. By cleaving NIK (NF-κB inducing kinase), the API2-MALT1 protease generates a c-terminal fragment of NIK that is resistant to proteasomal degradation and thereby increases noncanonical NF-κB signaling. By cleaving LIMA1α (LIM domain and actin-binding protein 1), the tumor-suppressing properties of this protein are diminished, and it believed that the remaining fragment has oncogenic properties and enhances cell proliferation, colony formation, and cell adhesion. Cleavage of Regnase 1 (Regulatory RNase 1, also known as MCPIP-1 or Zc3h12a), and roquin-1 (also known as RC3H1) is believed to result in the stabilization of mRNAs, including those of cytokines, chemokines, and costimulatory proteins such as ICOS, OX40, and TNF. This activity may be independent of MALT1 activity in the NF-κB and JNK pathways. See, e.g., Afonina, et al. FEBS J. 282.17 (2015): 3286-3297 Klein et al. Nat. Comm. 6.1 (2015): 1-17, Baens, et al. PloS one 9.8 (2014): e103774, and Juilland, Front. Immunol. 9 (2018): 1927. MALT1 is also involved in oncogenic BCR signalling in ibrutinib-responsive cell lines and biopsie samples, coordinated by a multiprotein supercomplex formed by MYD88, TLR9 and the BCR (hereafter termed the My-T-BCR supercomplex). The My-T-BCR supercomplex co-localizes with mTOR on endolysosomes, where it drives pro-survival NF-κB and mTOR signalling. See Phelan et al., Nature 2018 Aug; 560(7718):387-391.

Accordingly, inhibition of MALT1 can provide beneficial effects to many types of disorders associated with aberrant signaling in the NF-κB pathway or JNK pathway. For example, inhibition of MALT1 can decrease flux through the NF-κB or JNK pathways resulting from one or more of:
(1) An inactivated tumor suppressor gene. Non-limiting examples of tumor suppressor genes that can be inactivated include BRCA1 and p53 (e.g., p53H61L or 1123T). See, e.g., Sau, et al. Cell Stem Cell 19.1 (2016): 52-65, Xia, et al. Cancer Immunol. Res. 2.9 (2014): 823-830, Johansson, et al. Oncotarget 7.38 (2016): 62627.
(2) A dysregulated cell surface receptor. Non-limiting examples of cell surface receptors include HER1 and HER2. See, e.g., Xia, et al. Cancer Immunol. Res. 2.9 (2014): 823-830 and Pan, Mol. Cancer Res. 14.1 (2016): 93-102.
(3) Dysregulation of one or more components of a CBM complex. Non-limiting examples of components of a CBM complex include MALT1, CARD11, CARD14, CARD10, CARD9, and BCL10.
(4) Dysregulation of one or more substrates of a MALT1 protease (e.g., a wild-type MALT1 protease or a dysregulated MALT1 protease). Non-limiting examples of substrates of a MALT1 protease include BCL10, A20, CYLD, RelB, Regnase 1, roquin-1, HOIL1, NIK, and LIMA1α.
(5) Dysregulation of one or more components of the NF-κB pathway downstream of a CBM complex. Non-limiting examples of a component of the NF-κB pathway downstream of a CBM complex include TRAF6, IKKα, IKKβ, IKKγ (also called NEMO), IκBα, p50, p52, p65 (RelA), RelB, and c-Rel.
(6) Dysregulation of one or more components of the JNK pathway downstream of a CBM complex. Non-limiting examples of a component of the JNK pathway downstream of a CBM complex include JNK1 (Mitogen-Activated Protein Kinase 8), JNK2 (Mitogen-Activated Protein Kinase 9), JNK3 (Mitogen-Activated Protein Kinase 10), or an AP-1 transcription factor (e.g., a heterodimer of any of the c-Fos, c-Jun, ATF, or JDP families).
(7) Dysregulation of one or more fusion proteins caused by chromosome translocation of MALT1 gene. Non-limiting example includes the cIAP-MALT1 fusion protein.
(8) Dysregulation of one or more components of the My-T-BCR supercomplex. Non-limiting examples of a component of the My-T-BCR supercomplex include MYD88, TLR9, and mTOR.

The term “CBM complex pathway” as associated herein includes genes, transcripts, and proteins in a signaling pathway that includes a CBM. For example, many aspects of the NF-κB pathway are part of a CBM complex pathway. A CBM complex pathway can include, for example, cell surface receptors (e.g., CD28, BCR, HER1, and HER2), a signal transducer between a cell surface receptor and a CBM complex (e.g., a protein kinase C beta or protein kinase C theta), a component of a CBM complex (e.g., MALT1, CARD11, CARD14, CARD10, CARD9, or BCL10), substrates of a MALT1 protease (e.g., BCL10, A20, CYLD, RelB, Regnase 1, roquin-1, HOIL1, NIK, and LIMA1α), a component of the NF-κB pathway downstream of a CBM complex (e.g., TAK1, TRAF6, TAB1, TAB2, TAB3, MKK7, IKKα, IKKβ, IKKγ, IκBu, p50, p65 (RelA), or c-Rel), a component of the JNK pathway downstream of a CBM complex (e.g., JNK1, JNK2, JNK3, or an AP-1 transcription factor), or a components of the My-T-BCR supercomplex (e.g., MYD88, TLR9, or mTOR).

As used herein, the term “CBM complex pathway-associated disease or disorder” refers to diseases or disorders associated with or having a dysregulation of a gene in a CBM complex pathway, a protein in a CBM complex pathway, or the expression or activity or level of any (e.g., one or more) of the same (e.g., any of the types of dysregulation of a gene in a CBM complex pathway, a protein in a CBM complex pathway, or the expression or activity or level of any of the same, as described herein). Non-limiting examples of a CBM complex pathway-associated diseases or disorders include, for example, CBM-related primary immunodeficiency diseases, autoimmune disorders, multiple sclerosis, colitis, psoriasis, and cancer. See, e.g., McGuire, et al. J. Neuroinflamm. 11.1 (2014): 1-12, Lu, et al., Front. Immunol. 9 (2018): 2078, Jaworski, et al., EMBO J. 33.23 (2014): 2765-2781. Non-limiting examples of a CBM complex pathway-associated disease or disorder include MALT1-associated diseases or disorders such as MALT1-associated cancers, MALT1-associated autoimmune disorders, and MALT1-associated inflammatory disorders.

The term “CBM complex pathway-associated autoimmune disorder” as used herein refers to autoimmune disorders associated with or having a dysregulation of a CBM complex pathway gene, a CBM complex pathway protein, or the expression or activity or level of any (e.g., one or more) of the same (e.g., any of the types of dysregulation of a CBM complex pathway gene, a CBM complex pathway protein, or the expression or activity or level of any of the same described herein). Non-limiting examples of a CBM complex pathway-associated autoimmune disorders are described herein.

The term “CBM complex pathway-associated inflammatory disorder” as used herein refers to inflammatory disorders associated with or having a dysregulation of a CBM complex pathway gene, a CBM complex pathway protein, or the expression or activity or level of any (e.g., one or more) of the same (e.g., any of the types of dysregulation of a CBM complex pathway gene, a CBM complex pathway protein, or the expression or activity or level of any of the same described herein). Non-limiting examples of a CBM complex pathway-associated inflammatory disorders are described herein.

In some embodiments, a CBM complex pathway-associated disease or disorder is a CBM complex pathway-associated cancer, such as a CBM complex pathway cell surface receptor-associated cancer (e.g., a CD28-associated cancer, a BCR-associated cancer, a HER1-associated cancer, or a HER2-associated cancer), a cancer associated with a signal transducer between a cell surface receptor and a CBM complex (e.g., a protein kinase C beta (PKCβ)-associated cancer or a protein kinase C theta (PCKθ)-associated cancer), a component of a CBM complex-associated cancer (e.g., a MALT1-associated cancer, a CARD11-associated cancer, a CARD14-associated cancer, a CARD10-associated cancer, a CARD9-associated cancer, or a BCL10-associated cancer), a MALT1 protease substrate-associated cancer (e.g., a BCL10-associated cancer, an A20-associated cancer, a CYLD-associated cancer, a RelB-associated cancer, a Regnase 1-associated cancer, a roquin-1-associated cancer, a HOIL1-associated cancer, a NIK associated cancer, or a LIMA1α-associated cancer), a cancer associated with a component of the NF-1B pathway downstream of a CBM complex (e.g., TAK1-associated cancer, a TRAF6-associated cancer, a TAB1-associated cancer, a TAB2-associated cancer, a TAB3-associated cancer, a MKK7-associated cancer, an IKKα-associated cancer, an IKKβ-associated cancer, an IKKγ-associated cancer, an IκBα-associated cancer, a p50-associated cancer, a p65 (RelA)-associated cancer, or a c-Rel-associated cancer), a cancer associated with a component of the JNK pathway downstream of a CBM complex (e.g., a JNK1-associated cancer, a JNK2-associated cancer, a JNK3-associated cancer, or an AP-1 transcription factor-associated cancer), a MYD88-associated cancer, or a combination thereof.

The term “CBM complex pathway-associated cancer” as used herein refers to cancers associated with or having a dysregulation of a gene in a CBM complex pathway, a protein in a CBM complex pathway, or the expression or activity or level of any (e.g., one or more) of the same (e.g., any of the types of dysregulation of a gene in a CBM complex pathway, a protein in a CBM complex pathway, or the expression or activity or level of any of the same, as described herein) (e.g., upon diagnosis or after developing resistance to previous therapies. Non-limiting examples of a CBM complex pathway-associated cancer are described herein. In some embodiments, a CBM pathway-associated cancer can be a CBM complex pathway cell surface receptor-associated cancer (e.g., a CD28-associated cancer, a BCR-associated cancer, a HER1-associated cancer, or a HER2-associated cancer), a cancer associated with a signal transducer between a cell surface receptor and a CBM complex (e.g., a protein kinase C beta (PKCβ)-associated cancer or a protein kinase C theta (PCKθ)-associated cancer, a component of a CBM complex-associated cancer (e.g., a MALT1-associated cancer, a CARD11-associated cancer, a CARD14-associated cancer, a CARD10-associated cancer, a CARD9-associated cancer, or a BCL10-associated cancer), a MALT1 protease substrate-associated cancer (e.g., a BCL10-associated cancer, an A20-associated cancer, a CYLD-associated cancer, a RelB-associated cancer, a Regnase 1-associated cancer, a roquin-1-associated cancer, a HOIL1-associated cancer, a NIK associated cancer, or a LIMA1α-associated cancer), a cancer associated with a component of the NF-κB pathway downstream of a CBM complex (e.g., TAK1-associated cancer, a TRAF6-associated cancer, a TAB1-associated cancer, a TAB2-associated cancer, a TAB3-associated cancer, a MKK7-associated cancer, an IKKα-associated cancer, an IKKβ-associated cancer, an IKKγ-associated cancer, an IκBα-associated cancer, a p50-associated cancer, a p65 (RelA)-associated cancer, or a c-Rel-associated cancer), a cancer associated with a component of the JNK pathway downstream of a CBM complex (e.g., a JNK1-associated cancer, a JNK2-associated cancer, a JNK3-associated cancer, or an AP-1 transcription factor-associated cancer), or a combination thereof.

In some embodiments, a dysregulation can be a dysregulation that results in aberrant activation of a gene, protein, or expression or activity or level of any of the same. Activation can be through any appropriate mechanism, including, but not limited to, gene amplification, activating mutation, activating translocation, transcriptional activation, epigenetic alteration, and/or overexpression of the protein product of the oncogene. In some embodiments, a dysregulation can be a dysregulation that results in aberrant inactivation of a gene, protein, or expression or activity or level of any of the same. Inactivation can be through any appropriate mechanism, including, but not limited to, gene deletion, inactivating mutation, inactivating translocation, transcriptional silencing, epigenetic alteration, and degradation of mRNA and/or protein products of the gene. Typically, as used herein, a dysregulation, whether it be activation or inactivation, is a dysregulation that results in increased signaling through the NF-κB or JNK signaling pathways.

The term “wild-type” describes a nucleic acid (e.g., a MALT1 gene or a MALT1 mRNA) or protein (e.g., a MALT1 protein) that is found in a subject that does not have a disease or disorder associated with the nucleic acid or the protein (e.g., the MALT1 gene, MALT1 mRNA, or MALT1 protein) (and optionally also does not have an increased risk of developing a disease or disorder associated with the nucleic acid or the protein and/or is not suspected of having a disease or disorder associated with the gene or the protein), or is found in a cell or tissue from a subject that does not have a disease or disorder associated with the gene or the protein (e.g., a MALT1-associated cancer, autoimmune disorder, or inflammatory disorder) (and optionally also does not have an increased risk of developing a disease or disorder associated with the nucleic acid or the protein and/or is not suspected of having a disease or disorder associated with the nucleic acid or the protein.

In some embodiments, the subject has been identified or diagnosed as having a cancer with a dysregulation of a CBM complex pathway-associated gene (e.g., a MALT1 gene), a CBM complex pathway-associated protein (e.g., a MALT1 protein), or expression or activity, or level of any of the same (a CBM complex pathway-associated-associated cancer) (e.g., as determined using a regulatory agency-approved, e.g., FDA-approved, assay or kit). In some embodiments, the subject has a cancer resistant to one or more previous therapies. In some embodiments, the subject has a tumor that is positive for a dysregulation of a CBM complex pathway-associated gene (e.g., a MALT1 gene), a CBM complex pathway-associated protein (e.g., a MALT1 protein), or expression or activity, or level of any of the same (e.g., as determined using a regulatory agency-approved, e.g., FDA-approved, assay or kit). The subject can be a subject with a tumor(s) that is positive for a dysregulation of a CBM complex pathway-associated gene (e.g., a MALT1 gene), a CBM complex pathway-associated protein (e.g., a MALT1 protein), or expression or activity, or level of any of the same (e.g., identified as positive using a regulatory agency-approved, e.g., FDA-approved, assay or kit). The subject can be a subject whose tumors have a dysregulation of a CBM complex pathway-associated gene (e.g., a MALT1 gene), a CBM complex pathway-associated protein (e.g., a MALT1 protein), or expression or activity, or a level of the same (e.g., where the tumor is identified as such using a regulatory agency-approved, e.g., FDA-approved, kit or assay). In some embodiments, the subject has a tumor resistant to one or more previous therapies. In some embodiments, the subject is suspected of having a CBM complex pathway-associated-associated cancer. In some embodiments, the subject has a tumor that is suspected of being resistant to one or more previous therapies. In some embodiments, the subject has a clinical record indicating that the subject has a tumor that has a dysregulation of a CBM complex pathway-associated gene (e.g., a MALT1 gene), a CBM complex pathway-associated protein (e.g., a MALT1 protein), or expression or activity, or level of any of the same (and optionally the clinical record indicates that the subject should be treated with any of the compositions provided herein). In some embodiments, the subject is a pediatric subject. In some embodiments, the subject has a clinical record indicating that the subject has a tumor resistant to one or more previous therapies. In some embodiments, the subject has been identified or diagnosed as having a cancer that, based on histological examination, is determined to be associated with a dysregulation of a CBM complex pathway-associated gene (e.g., a MALT1 gene), a CBM complex pathway-associated protein (e.g., a MALT1 protein), or expression or activity, or level of any of the same (a CBM complex pathway-associated-associated cancer).

In some embodiments, the subject has been identified or diagnosed as having an autoimmune disorder with a dysregulation of a CBM complex pathway-associated gene (e.g., a MALT1 gene), a CBM complex pathway-associated protein (e.g., a MALT1 protein), or expression or activity, or level of any of the same (a CBM complex pathway-associated-associated autoimmune disorder) (e.g., as determined using a regulatory agency-approved, e.g., FDA-approved, assay or kit). In some embodiments, the subject has a tumor that is positive for a dysregulation of a CBM complex pathway-associated gene (e.g., a MALT1 gene), a CBM complex pathway-associated protein (e.g., a MALT1 protein), or expression or activity, or level of any of the same (e.g., as determined using a regulatory agency-approved, e.g., FDA-approved, assay or kit). In some embodiments, the subject is suspected of having a CBM complex pathway-associated-associated autoimmune disorder. In some embodiments, the subject has a clinical record indicating that the subject has a tumor that has a dysregulation of a CBM complex pathway-associated gene (e.g., a MALT1 gene), a CBM complex pathway-associated protein (e.g., a MALT1 protein), or expression or activity, or level of any of the same (and optionally the clinical record indicates that the subject should be treated with any of the compositions provided herein). In some embodiments, the subject is a pediatric subject. In some embodiments, the subject has been identified or diagnosed as having an autoimmune disorder that, based on histological examination, is determined to be associated with a dysregulation of a CBM complex pathway-associated gene (e.g., a MALT1 gene), a CBM complex pathway-associated protein (e.g., a MALT1 protein), or expression or activity, or level of any of the same (a CBM complex pathway-associated-associated autoimmune disorder).

In some embodiments, the subject has been identified or diagnosed as having an inflammatory disorder with a dysregulation of a CBM complex pathway-associated gene (e.g., a MALT1 gene), a CBM complex pathway-associated protein (e.g., a MALT1 protein), or expression or activity, or level of any of the same (a CBM complex pathway-associated-associated inflammatory disorder) (e.g., as determined using a regulatory agency-approved, e.g., FDA-approved, assay or kit). In some embodiments, the subject has a tumor that is positive for a dysregulation of a CBM complex pathway-associated gene (e.g., a MALT1 gene), a CBM complex pathway-associated protein (e.g., a MALT1 protein), or expression or activity, or level of any of the same (e.g., as determined using a regulatory agency-approved, e.g., FDA-approved, assay or kit). In some embodiments, the subject is suspected of having a CBM complex pathway-associated-associated inflammatory disorder. In some embodiments, the subject has a clinical record indicating that the subject has a tumor that has a dysregulation of a CBM complex pathway-associated gene (e.g., a MALT1 gene), a CBM complex pathway-associated protein (e.g., a MALT1 protein), or expression or activity, or level of any of the same (and optionally the clinical record indicates that the subject should be treated with any of the compositions provided herein). In some embodiments, the subject is a pediatric subject. In some embodiments, the subject has been identified or diagnosed as having an inflammatory disorder that, based on histological examination, is determined to be associated with a dysregulation of a CBM complex pathway-associated gene (e.g., a MALT1 gene), a CBM complex pathway-associated protein (e.g., a MALT1 protein), or expression or activity, or level of any of the same (a CBM complex pathway-associated-associated inflammatory disorder).

The term “CBM complex pathway cell surface receptor-associated cancer” as used herein refers to cancers associated with or having a dysregulation of a gene, a protein, or the expression or activity or level of any (e.g., one or more) of the same associated with a CBM complex pathway cell surface receptor. In some embodiments, a CBM complex pathway cell surface receptor-associated cancer is selected from the group consisting of a CD28-associated cancer, a BCR-associated cancer, a HER1-associated cancer, a HER2-associated cancer, and combinations thereof.

The term “*-associated cancer” as used herein refers to cancers associated with or having a dysregulation of a * gene, a * protein, or the expression or activity or level of any (e.g., one or more) of the same (e.g., any of the types of dysregulation of a * gene, a * protein, or the expression or activity or level of any of the same described herein), where “*” refers to a particular CBM complex pathway gene or protein, described herein. In some embodiments, the *-associated cancer is selected from the group consisting of: CD28-associated cancer, BCR-associated cancer, HER1-associated cancer, HER2-associated cancer, PKCβ-associated cancer, PKCθ-associated cancer, MALT1-associated cancer, CARD11-associated cancer, CARD14-associated cancer, A20-associated cancer, CYLD-associated cancer, RelB-associated cancer, HOIL1-associated cancer, NIK-associated cancer, Regnase 1-associated cancer, LIMA1α-associated cancer, roquin-1-associated cancer, TRAF6-associated cancer, TAK1-associated cancer, TAB1-associated cancer, TAB2-associated cancer, TAB3-associated cancer, MKK7-associated cancer, IKKα-associated cancer, IKKβ-associated cancer, IKKγ-associated cancer, IκBα-associated cancer, p50-associated cancer, p65-associated cancer, c-Rel-associated cancer, JNK1-associated cancer, JNK2-associated cancer, JNK3-associated cancer, MYD88 transcription factor-associated cancer, and an AP-1 transcription factor-associated cancer. In some embodiments, the *-associated cancer is a CD28-associated cancer. In some embodiments, the *-associated cancer is a BCR-associated cancer. In some embodiments, the *-associated cancer is a HER1-associated cancer. In some embodiments, the *-associated cancer is a HER2-associated cancer. In some embodiments, the *-associated cancer is a PKCβ-associated cancer. In some embodiments, the *-associated cancer is a PKCθ-associated cancer. In some embodiments, the *-associated cancer is a MALT1-associated cancer. In some embodiments, the *-associated cancer is a CARD11-associated cancer. In some embodiments, the *-associated cancer is a CARD14-associated cancer. In some embodiments, the *-associated cancer is an A20-associated cancer. In some embodiments, the *-associated cancer is a CYLD-associated cancer. In some embodiments, the *-associated cancer is a RelB-associated cancer. In some embodiments, the *-associated cancer is a HOIL1-associated cancer. In some embodiments, the *-associated cancer is a NIK-associated cancer. In some embodiments, the *-associated cancer is a Regnase 1-associated cancer. In some embodiments, the *-associated cancer is a LIMA1α-associated cancer. In some embodiments, the *-associated cancer is a roquin-1-associated cancer. In some embodiments, the *-associated cancer is a TRAF6-associated cancer. In some embodiments, the *-associated cancer is a TAK1-associated cancer. In some embodiments, the *-associated cancer is a TAB1-associated cancer. In some embodiments, the *-associated cancer is a TAB2-associated cancer. In some embodiments, the *-associated cancer is a TAB3-associated cancer. In some embodiments, the *-associated cancer is a MKK7-associated cancer, and an IKKα-associated cancer. In some embodiments, the *-associated cancer is an IKKβ-associated cancer. In some embodiments, the *-associated cancer is an IKKγ-associated cancer. In some embodiments, the *-associated cancer is an IκBα-associated cancer. In some embodiments, the *-associated cancer is a p50-associated cancer. In some embodiments, the *-associated cancer is a p65-associated cancer. In some embodiments, the *-associated cancer is a c-Rel-associated cancer. In some embodiments, the *-associated cancer is a JNK1-associated cancer. In some embodiments, the *-associated cancer is a JNK2-associated cancer. In some embodiments, the *-associated cancer is a JNK3-associated cancer. In some embodiments, the *-associated cancer is a AP-1 transcription factor-associated cancer. In some embodiments, the *-associated cancer is a MYD88 transcription factor-associated cancer.

The phrase “dysregulation of a * gene, a * protein, or the expression or activity or level of any of the same” (where * is a particular CBM complex pathway gene or protein, described herein) refers to a genetic mutation (e.g., a chromosomal translocation that results in the expression of a fusion protein including a * domain and a fusion partner, a mutation in a * gene that results in the expression of a * protein that includes a deletion of at least one amino acid as compared to a wild-type * protein, a mutation in a * gene that results in the expression of a * protein with one or more point mutations as compared to a wild-type * protein, a mutation in a * gene that results in the expression of a * protein with at least one inserted amino acid as compared to a wild-type * protein, a gene duplication that results in an increased level of * protein in a cell, or a mutation in a regulatory sequence (e.g., a promoter and/or enhancer) that results in an increased level of * protein in a cell), an alternative spliced version of a * mRNA that results in a * protein having a deletion of at least one amino acid in the * protein as compared to the wild-type * protein, or increased expression (e.g., increased levels) of a wild-type * protein in a mammalian cell due to aberrant cell signaling and/or dysregulated autocrine/paracrine signaling (e.g., as compared to a control non-cancerous cell). As a further example, an increased copy number of the * gene can result in overexpression of the * protein. For example, a dysregulation of a * gene, a * protein, or expression or activity, or level of any of the same, can be the result of a gene or chromosome translocation which results in the expression of a fusion protein that contains a first portion of *, and a second portion of a partner protein (i.e., that is not *). In some examples, dysregulation of a * gene, a * protein, or expression or activity or level of any of the same can be a result of a gene translocation of one * gene with another non-*gene. In some embodiments, the * gene, a * protein, or the expression or activity or level of any of the same is selected from the group consisting of: CD28, BCR, HER1, HER2, PKCβ, PKCθ, MALT1, CARD11, CARD14, A20, CYLD, RelB, HOIL1, NIK, Regnase 1, LIMA1α, roquin-1, TRAF6, TAK1, TAB1, TAB2, TAB3, MKK7, IKKα, IKKβ, IKKγ, IκBα, p50, p65, c-Rel, JNK1, JNK2, JNK3, MYD88, and an AP-1 transcription factor. In some embodiments, the * gene or * protein is CD28. In some embodiments, the * gene or * protein is BCR. In some embodiments, the * gene or * protein is HER1. In some embodiments, the * gene or * protein is HER2. In some embodiments, the * gene or * protein is PKCβ. In some embodiments, the * gene or * protein is PKCθ. In some embodiments, the * gene or * protein is MALT1. In some embodiments, the * gene or * protein is CARD11. In some embodiments, the * gene or * protein is CARD14. In some embodiments, the * gene or * protein is A20. In some embodiments, the * gene or * protein is CYLD. In some embodiments, the * gene or * protein is RelB. In some embodiments, the * gene or * protein is HOIL1. In some embodiments, the * gene or * protein is NIK. In some embodiments, the * gene or * protein is Regnase 1. In some embodiments, the * gene or * protein is LIMA1α. In some embodiments, the * gene or * protein is roquin-1. In some embodiments, the * gene or * protein is TRAF6. In some embodiments, the * gene or * protein is TAK1. In some embodiments, the * gene or * protein is TAB1. In some embodiments, the * gene or * protein is TAB2. In some embodiments, the * gene or * protein is TAB3. In some embodiments, the * gene or * protein is MKK7. In some embodiments, the * gene or * protein is IKKα. In some embodiments, the * gene or * protein is IKKβ. In some embodiments, the * gene or * protein is IKKγ. In some embodiments, the * gene or * protein is IκBα. In some embodiments, the * gene or * protein is p50. In some embodiments, the * gene or * protein is p65. In some embodiments, the * gene or * protein is c-Rel. In some embodiments, the * gene or * protein is JNK1. In some embodiments, the * gene or * protein is JNK2. In some embodiments, the * gene or * protein is JNK3. In some embodiments, the * gene or * protein is MYD88 transcription factor. In some embodiments, the * gene or * protein is AP-1 transcription factor.

In some embodiments, dysregulation of a * gene, a * protein, or expression or activity, or level of any of the same, can be a mutation in a * gene that encodes a * protein that is constitutively active or has increased activity as compared to a protein encoded by a * gene that does not include the mutation. In some embodiments, an increased copy number of the * gene can result in overexpression of * protein. In some embodiments, the * gene, * protein, or expression or activity, or level of any of the same, is CD28. In some embodiments, the * gene, * protein, or expression or activity, or level of any of the same, is BCR. In some embodiments, the * gene, * protein, or expression or activity, or level of any of the same, is HER1. In some embodiments, the * gene, * protein, or expression or activity, or level of any of the same, is HER2. In some embodiments, the * gene, * protein, or expression or activity, or level of any of the same, is PKCβ. In some embodiments, the * gene, * protein, or expression or activity, or level of any of the same, is PKCθ. In some embodiments, the * gene, * protein, or expression or activity, or level of any of the same, is CARD14. In some embodiments, the * gene, * protein, or expression or activity, or level of any of the same, is CARD9. In some embodiments, the * gene, * protein, or expression or activity, or level of any of the same, is CARD10. In some embodiments, the * gene, * protein, or expression or activity, or level of any of the same, is CARD11. In some embodiments, the * gene, * protein, or expression or activity, or level of any of the same, is MALT1.

As another example, a dysregulation of an * gene, an * protein, or expression or activity, or level of any of the same, can be a mutation in an * gene that encodes an * protein that is constitutively inactive or has decreased activity as compared to a protein encoded by an * gene that does not include the mutation. In some embodiments, the * gene, * protein, or expression or activity, or level of any of the same, is A20. In some embodiments, the * gene, * protein, or expression or activity, or level of any of the same, is CYLD. In some embodiments, the * gene, * protein, or expression or activity, or level of any of the same, is RelB. In some embodiments, the * gene, * protein, or expression or activity, or level of any of the same, is HOIL1. In some embodiments, the * gene, * protein, or expression or activity, or level of any of the same, is NIK.

Diseases or disorders “associated” with a particular gene or protein described herein refer to diseases or disorder associated with or having a dysregulation of the particular gene, the particular protein, or the expression or activity or level of any (e.g., one or more) of the same (e.g., any of the types of dysregulation of the particular gene, the particular protein, or the expression or activity or level of any of the same described herein). Non-limiting examples of such diseases or disorders are described herein. Likewise, cancers “associated” with a particular gene or protein described herein refer to cancers associated with or having a dysregulation of the particular gene, the particular protein, or the expression or activity or level of any (e.g., one or more) of the same (e.g., any of the types of dysregulation of the particular gene, the particular protein, or the expression or activity or level of any of the same described herein). Non-limiting examples of such cancers are described herein.

Exemplary sequences of the proteins described herein are shown below.

An exemplary sequence of human CD28 is shown below:

(UniParc Accession No. UPI0000043F4D)
SEQ ID NO: 1
MLRLLLALNLFPSIQVTGNKILVKQSPMLVAYDNAVNLSCKYSYNLFSR
EFRASLHKGLDSAVEVCVVYGNYSQQLQVYSKTGFNCDGKLGNESVTFY
LONLYVNQTDIYFCKIEVMYPPPYLDNEKSNGTIIHVKGKHLCPSPLFP
GPSKPFWVLVVVGGVLACYSLLVTVAFIIFWVRSKRSRLLHSDYMNMTP
RRPGPTRKHYQPYAPPRDFAAYRS

Non-limiting examples of dysregulation of a CD28 gene or a CD28 protein can be found in, for example, Rohr, et al., Leukemia 30.5 (2016): 1062-1070, Yoo, et al., Haematologica 101.6 (2016): 757-763, and Lee. et al., Haematologica 100.12 (2015): e505.

An exemplary sequence of human BCR is shown below:

(UniParc Accession No. UPI000016A088)
SEQ ID NO: 2
MVDPVGFAEAWKAQFPDSEPPRMELRSVGDIEQELERCKASIRRLEQEVNQERFRMIYLQ
TLLAKEKKSYDRQRWGFRRAAQAPDGASEPRASASRPQPAPADGADPPPAEEPEARPDGE
GSPGKARPGTARRPGAAASGERDDRGPPASVAALRSNFERIRKGHGQPGADAEKPFYVNV
EFHHERGLVKVNDKEVSDRISSLGSQAMQMERKKSQHGAGSSVGDASRPPYRGRSSESSC
GVDGDYEDAELNPRFLKDNLIDANGGSRPPWPPLEYQPYQSIYVGGMMEGEGKGPLLRSQ
STSEQEKRLTWPRRSYSPRSFEDCGGGYTPDCSSNENLTSSEEDFSSGQSSRVSPSPTTY
RMFRDKSRSPSQNSQQSFDSSSPPTPQCHKRHRHCPVVVSEATIVGVRKTGQIWPNDGEG
AFHGDADGSFGTPPGYGCAADRAEEQRRHQDGLPYIDDSPSSSPHLSSKGRGSRDALVSG
ALESTKASELDLEKGLEMRKWVLSGILASEETYLSHLEALLLPMKPLKAAATTSQPVLTS
QQIETIFFKVPELYEIHKEFYDGLFPRVQQWSHQQRVGDLFQKLASQLGVYRAFVDNYGV
AMEMAEKCCQANAQFAEISENLRARSNKDAKDPTTKNSLETLLYKPVDRVTRSTLVLHDL
LKHTPASHPDHPLLQDALRISQNFLSSINEEITPRRQSMTVKKGEHRQLLKDSEMVELVE
GARKLRHVFLFTDLLLCTKLKKQSGGKTQQYDCKWYIPLTDLSFQMVDELEAVPNIPLVP
DEELDALKIKISQIKNDIQREKRANKGSKATERLKKKLSEQESLLLLMSPSMAFRVHSRN
GKSYTFLISSDYERAEWRENIREQQKKCFRSFSLTSVELQMLINSCVKLQTVHSIPLTIN
KEDDESPGLYGFLNVIVHSATGFKQSSNLYCTLEVDSFGYFVNKAKTRVYRDTAEPNWNE
EFEIELEGSQTLRILCYEKCYNKTKIPKEDGESTDRLMGKGQVQLDPQALQDRDWQRTVI
AMNGIEVKLSVKFNSREFSLKRMPSRKQTGVFGVKIAVVTKRERSKVPYIVRQCVEEIER
RGMEEVGIYRVSGVATDIQALKAAFDVNNKDVSVMMSEMDVNAIAGTLKLYFRELPEPLF
TDEFYPNFAEGIALSDPVAKESCMLNLLLSLPEANLLTFLFLLDHLKRVAEKEAVNKMSL
HNLATVFGPTLLRPSEKESKLPANPSQPITMTDSWSLEVMSQVQVLLYFLQLEAIPAPDS
KRQSILFSTEV

Non-limiting examples of dysregulation of a BCR gene or a BCR protein (e.g., a BCR-ABL fusion) can be found in, for example, Yang and Fu, Crit. Rev. Oncol./Hematol. 93.3 (2015): 277-292, Weisberg, et al. Nat. Rev. Cancer 7.5 (2007): 345-356, and Jabbour, et al. Cancer 117.9 (2011): 1800-1811.

An exemplary sequence of human HER1 is shown below:

(UniParc Accession No. UPI000003E750)
SEQ ID NO: 3
MRPSGTAGAALLALLAALCPASRALEEKKVCQGTSNKLTQLGTFEDHFLSLQRMFNNCEV
VLGNLEITYVQRNYDLSFLKTIQEVAGYVLIALNTVERIPLENLQIIRGNMYYENSYALA
VLSNYDANKTGLKELPMRNLQEILHGAVRFSNNPALCNVESIQWRDIVSSDFLSNMSMDF
QNHLGSCQKCDPSCPNGSCWGAGEENCQKLTKIICAQQCSGRCRGKSPSDCCHNQCAAGC
TGPRESDCLVCRKFRDEATCKDTCPPLMLYNPTTYQMDVNPEGKYSFGATCVKKCPRNYV
VTDHGSCVRACGADSYEMEEDGVRKCKKCEGPCRKVCNGIGIGEFKDSLSINATNIKHFK
NCTSISGDLHILPVAFRGDSFTHTPPLDPQELDILKTVKEITGFLLIQAWPENRTDLHAF
ENLEIIRGRTKQHGQFSLAVVSLNITSLGLRSLKEISDGDVIISGNKNLCYANTINWKKL
FGTSGQKTKIISNRGENSCKATGQVCHALCSPEGCWGPEPRDCVSCRNVSRGRECVDKCN
LLEGEPREFVENSECIQCHPECLPQAMNITCTGRGPDNCIQCAHYIDGPHCVKTCPAGVM
GENNTLVWKYADAGHVCHLCHPNCTYGCTGPGLEGCPTNGPKIPSIATGMVGALLLLLVV
ALGIGLFMRRRHIVRKRTLRRLLQERELVEPLTPSGEAPNQALLRILKETEFKKIKVLGS
GAFGTVYKGLWIPEGEKVKIPVAIKELREATSPKANKEILDEAYVMASVDNPHVCRLLGI
CLISTVQLITQLMPFGCLLDYVREHKDNIGSQYLLNWCVQIAKGMNYLEDRRLVHRDLAA
RNVLVKTPQHVKITDFGLAKLLGAEEKEYHAEGGKVPIKWMALESILHRIYTHQSDVWSY
GVTVWELMTFGSKPYDGIPASEISSILEKGERLPQPPICTIDVYMIMVKCWMIDADSRPK
FRELIIEFSKMARDPQRYLVIQGDERMHLPSPTDSNFYRALMDEEDMDDVVDADEYLIPQ
QGFFSSPSTSRTPLLSSLSATSNNSTVACIDRNGLQSCPIKEDSFLQRYSSDPTGALTED
SIDDTFLPVPEYINQSVPKRPAGSVQNPVYHNQPLNPAPSRDPHYQDPHSTAVGNPEYLN
TVQPTCVNSTFDSPAHWAQKGSHQISLDNPDYQQDFFPKEAKPNGIFKGSTAENAEYLRV
APQSSEFIGA

Non-limiting examples of dysregulation of a HER1 gene or a HER1 protein can be found in, for example, Zhang, et al., Oncotarget 7.48 (2016): 78985, Ellison, et al., Journal of Clinical Pathology 66.2 (2013): 79-89, Midha, et al., American Journal of Cancer Research 5.9 (2015): 2892, and Yamamoto, et al., Lung Cancer 63.3 (2009): 315-321.

An exemplary sequence of human HER2 is shown below:

(UniParc Accession No. UPI000003F55F)
SEQ ID NO: 4
MELAALCRWGLLLALLPPGAASTQVCTGTDMKLRLPASPETHLDMLRHLYQGCQVVQGNL
ELTYLPINASLSFLQDIQEVQGYVLIAHNQVRQVPLQRLRIVRGTQLFEDNYALAVLDNG
DPLNNTTPVTGASPGGLRELQLRSLTEILKGGVLIQRNPQLCYQDTILWKDIFHKNNQLA
LTLIDTNRSRACHPCSPMCKGSRCWGESSEDCQSLTRTVCAGGCARCKGPLPTDCCHEQC
AAGCTGPKHSDCLACLHFNHSGICELHCPALVTYNTDTFESMPNPEGRYTFGASCVTACP
YNYLSTDVGSCTLVCPLHNQEVTAEDGTQRCEKCSKPCARVCYGLGMEHLREVRAVTSAN
IQEFAGCKKIFGSLAFLPESFDGDPASNTAPLQPEQLQVFETLEEITGYLYISAWPDSLP
DLSVFQNLQVIRGRILHNGAYSLTLQGLGISWLGLRSLRELGSGLALIHHNTHLCFVHTV
PWDQLFRNPHQALLHTANRPEDECVGEGLACHQLCARGHCWGPGPTQCVNCSQFLRGQEC
VEECRVLQGLPREYVNARHCLPCHPECQPQNGSVTCFGPEADQCVACAHYKDPPFCVARC
PSGVKPDLSYMPIWKFPDEEGACQPCPINCTHSCVDLDDKGCPAEQRASPLTSIISAVVG
ILLVVVLGVVFGILIKRRQQKIRKYTMRRLLQETELVEPLTPSGAMPNQAQMRILKETEL
RKVKVLGSGAFGTVYKGIWIPDGENVKIPVAIKVLRENTSPKANKEILDEAYVMAGVGSP
YVSRLLGICLTSTVQLVTQLMPYGCLLDHVRENRGRLGSQDLLNWCMQIAKGMSYLEDVR
LVHRDLAARNVLVKSPNHVKITDFGLARLLDIDETEYHADGGKVPIKWMALESILRRRFT
HQSDVWSYGVTVWELMTFGAKPYDGIPAREIPDLLEKGERLPQPPICTIDVYMIMVKCWM
IDSECRPRFRELVSEFSRMARDPQRFVVIQNEDLGPASPLDSTFYRSLLEDDDMGDLVDA
EEYLVPQQGFFCPDPAPGAGGMVHHRHRSSSTRSGGGDLTLGLEPSEEEAPRSPLAPSEG
AGSDVFDGDLGMGAAKGLQSLPTHDPSPLQRYSEDPTVPLPSETDGYVAPLTCSPQPEYV
NQPDVRPQPPSPREGPLPAARPAGATLERPKTLSPGKNGVVKDVFAFGGAVENPEYLTPQ
GGAAPQPHPPPAFSPAFDNLYYWDQDPPERGAPPSTFKGTPTAENPEYLGLDVPV

Non-limiting examples of dysregulation of a HER2 gene or a HER2 protein can be found, for example, Petrelli, Fausto, et al., Breast Cancer Research and Treatment 166.2 (2017): 339-349, Yan, et al., Cancer and Metastasis Reviews 34.1 (2015): 157-164, Koshkin, et al., Bladder Cancer 5.1 (2019): 1-12, and Connell, et al., ESMO Open 2.5 (2017).

The term “cancer associated with a signal transducer between a cell surface receptor and a CBM complex” as used herein refers to cancers associated with or having a dysregulation of a gene, a protein, or the expression or activity or level of any (e.g., one or more) of the same associated with a signal transducer between a cell surface receptor and a CBM complex. In some embodiments, a cancer associated with a signal transducer between a cell surface receptor and a CBM complex is selected from the group consisting of a PKCβ-associated cancer, PCKθ-associated cancer, and a combination thereof. The cancers “associated” with a particular gene or protein described in this paragraph refer to cancers associated with or having a dysregulation of the particular gene, the particular protein, or the expression or activity or level of any (e.g., one or more) of the same (e.g., any of the types of dysregulation of the particular gene, the particular protein, or the expression or activity or level of any of the same described herein). Non-limiting examples of such cancers are described herein.

An exemplary sequence of human PKCβ is shown below:

(UniParc Accession No. UPI000012DF67)
SEQ ID NO: 5
MADPAAGPPPSEGEESTVRFARKGALRQKNVHEVKNHKFTARFFKQPTFCSHCTDFIWGF
GKQGFQCQVCCFVVHKRCHEFVTFSCPGADKGPASDDPRSKHKFKIHTYSSPTFCDHCGS
LLYGLIHQGMKCDTCMMNVHKRCVMNVPSLCGTDHTERRGRIYIQAHIDRDVLIVLVRDA
KNLVPMDPNGLSDPYVKLKLIPDPKSESKQKTKTIKCSLNPEWNETFRFQLKESDKDRRL
SVEIWDWDLISRNDFMGSLSFGISELQKASVDGWFKLLSQEEGEYFNVPVPPEGSEANEE
LRQKFERAKISQGTKVPEEKTTNTVSKFDNNGNRDRMKLTDFNFLMVLGKGSFGKVMLSE
RKGTDELYAVKILKKDVVIQDDDVECTMVEKRVLALPGKPPFLTQLHSCFQTMDRLYFVM
EYVNGGDLMYHIQQVGRFKEPHAVFYAAEIAIGLFFLQSKGIIYRDLKLDNVMLDSEGHI
KIADFGMCKENIWDGVTTKTFCGTPDYIAPEIIAYQPYGKSVDWWAFGVLLYEMLAGQAP
FEGEDEDELFQSIMEHNVAYPKSMSKEAVAICKGLMTKHPGKRLGCGPEGERDIKEHAFF
RYIDWEKLERKEIQPPYKPKARDKRDTSNFDKEFTRQPVELTPTDKLFIMNLDQNEFAGF
SYINPEFVINV

An exemplary sequence of human PKCθ is shown below:

(UniParc Accession No. UPI000012DF74)
SEQ ID NO: 6
MSPFLRIGLSNFDCGSCQSCQGEAVNPYCAVLVKEYVESENGQMYIQKKPTMYPPWDSTF
DAHINKGRVMQIIVKGKNVDLISETTVELYSLAERCRKNNGKTEIWLELKPQGRMLMNAR
YFLEMSDTKDMNEFETEGFFALHQRRGAIKQAKVHHVKCHEFTATFFPQPTFCSVCHEFV
WGLNKQGYQCRQCNAAIHKKCIDKVIAKCTGSAINSRETMFHKERFKIDMPHRFKVYNYK
SPTFCEHCGTLLWGLARQGLKCDACGMNVHHRCQTKVANLCGINQKLMAEALAMIESTQQ
ARCLRDTEQIFREGPVEIGLPCSIKNEARPPCLPTPGKREPQGISWESPLDEVDKMCHLP
EPELNKERPSLQIKLKIEDFILHKMLGKGSFGKVFLAEFKKTNQFFAIKALKKDVVLMDD
DVECTMVEKRVLSLAWEHPFLTHMFCTFQTKENLFFVMEYLNGGDLMYHIQSCHKFDLSR
ATFYAAEIILGLQFLHSKGIVYRDLKLDNILLDKDGHIKIADFGMCKENMLGDAKINTFC
GTPDYIAPEILLGQKYNHSVDWWSFGVLLYEMLIGQSPFHGQDEEELFHSIRMDNPFYPR
WLEKEAKDLLVKLFVREPEKRLGVRGDIRQHPLFREINWEELERKEIDPPFRPKVKSPFD
CSNFDKEFLNEKPRLSFADRALINSMDQNMFRNFSFMNPGMERLIS

The term “component of a CBM complex-associated cancer” as used herein refers to cancers associated with or having a dysregulation of a gene, a protein, or the expression or activity or level of any (e.g., one or more) of the same associated with a component of a CBM complex. In some embodiments, a component of a CBM complex-associated cancer is selected from the group consisting of a MALT1-associated cancer, a CARD11-associated cancer, a CARD14-associated cancer, a CARD10-associated cancer, a CARD9-associated cancer, a BCL10-associated cancer, and combinations thereof. In some embodiments, a CBM complex-associated cancer is selected from the group consisting of a MALT1-associated cancer, a CARD11-associated cancer, a BCL10-associated cancer, and combinations thereof. The cancers “associated” with a particular gene or protein described in this paragraph refer to cancers associated with or having a dysregulation of the particular gene, the particular protein, or the expression or activity or level of any (e.g., one or more) of the same (e.g., any of the types of dysregulation of the particular gene, the particular protein, or the expression or activity or level of any of the same described herein). Non-limiting examples of such cancers are described herein.

The term “MALT1-associated autoimmune disorder” as used herein refers to autoimmune disorders associated with or having a dysregulation of a MALT1 gene, a MALT1 protein (also called herein MALT1 protease protein or MALT1 protease), or the expression or activity or level of any (e.g., one or more) of the same (e.g., any of the types of dysregulation of a MALT1 gene, a MALT1 protease, a MALT1 protease domain, or the expression or activity or level of any of the same described herein). Non-limiting examples of a MALT1-associated autoimmune disorders are described herein.

The term “MALT1-associated inflammatory disorder” as used herein refers to inflammatory disorders associated with or having a dysregulation of a MALT1 gene, a MALT1 protein (also called herein MALT1 protease protein or MALT1 protease), or the expression or activity or level of any (e.g., one or more) of the same (e.g., any of the types of dysregulation of a MALT1 gene, a MALT1 protease, a MALT1 protease domain, or the expression or activity or level of any of the same described herein). Non-limiting examples of a MALT1-associated inflammatory disorders are described herein.

The term “MALT1-associated cancer” as used herein refers to cancers associated with or having a dysregulation of a MALT1 gene, a MALT1 protein (also called herein MALT1 protease protein or MALT1 protease), or the expression or activity or level of any (e.g., one or more) of the same (e.g., any of the types of dysregulation of a MALT1 gene, a MALT1 protein, a MALT1 protease domain, or the expression or activity or level of any of the same described herein). Non-limiting examples of a MALT1-associated cancer are described herein.

The phrase “dysregulation of a MALT1 gene, a MALT1 protein, or the expression or activity or level of any of the same” refers to a genetic mutation (e.g., a chromosomal translocation that results in the expression of a fusion protein including a MALT1 protease domain and a fusion partner, a mutation in a MALT1 gene that results in the expression of a MALT1 protein that includes a deletion of at least one amino acid as compared to a wild-type MALT1 protein, a mutation in a MALT1 gene that results in the expression of a MALT1 protein with one or more point mutations as compared to a wild-type MALT1 protein, a mutation in a MALT1 gene that results in the expression of a MALT1 protein with at least one inserted amino acid as compared to a wild-type MALT1 protein, a gene duplication that results in an increased level of MALT1 protein in a cell, or a mutation in a regulatory sequence (e.g., a promoter and/or enhancer) that results in an increased level of MALT1 protein in a cell), an alternative spliced version of a MALT1 mRNA that results in a MALT1 protein having a deletion of at least one amino acid in the MALT1 protein as compared to the wild-type MALT1 protein, or increased expression (e.g., increased levels) of a wild-type MALT1 protein in a mammalian cell due to aberrant cell signaling and/or dysregulated autocrine/paracrine signaling (e.g., as compared to a control non-cancerous cell). As another example, a dysregulation of a MALT1 gene, a MALT1 protein, or expression or activity, or level of any of the same, can be a mutation in a MALT1 gene that encodes a MALT1 protein that is constitutively active or has increased activity as compared to a protein encoded by a MALT1 gene that does not include the mutation. As a further example, an increased copy number of the MALT1 gene can result in overexpression of MALT1 protease. For example, a dysregulation of a MALT1 gene, a MALT1 protein, or expression or activity, or level of any of the same, can be the result of a gene or chromosome translocation which results in the expression of a fusion protein that contains a first portion of MALT1 that includes a functional protease domain, and a second portion of a partner protein (i.e., that is not MALT1). In some examples, dysregulation of a MALT1 gene, a MALT1 protein, or expression or activity or level of any of the same can be a result of a gene translocation of one MALT1 gene with another non-MALT1 gene.

An exemplary sequence of human MALT1 is shown below:

(UniParc Accession No. UPI000004D05E)
SEQ ID NO: 7
MSLLGDPLQALPPSAAPTGPLLAPPAGAILNRLREPLLRRLSELLDQAPEGRGWRRLAEL
AGSRGRLRLSCLDLEQCSLKVLEPEGSPSLCLLKLMGEKGCTVTELSDFLQAMEHTEVLQ
LLSPPGIKITVNPESKAVLAGQFVKLCCRATGHPFVQYQWFKMNKEIPNGNISELIFNAV
HVKDAGFYVCRVNNNFTFEFSQWSQLDVCDIPESFQRSVDGVSESKLQICVEPTSQKLMP
GSTLVLQCVAVGSPIPHYQWFKNELPLTHETKKLYMVPYVDLEHQGTYWCHVYNDRDSQD
SKKVEIIIGRIDEAVECTEDELNNLGHPDNKEQTTDQPLAKDKVALLIGNMNYREHPKLK
APLVDVYELTNLLRQLDFKVVSLLDLTEYEMRNAVDEFLLLLDKGVYGLLYYAGHGYENF
GNSFMVPVDAPNPYRSENCLCVQNILKLMQEKETGLNVFLLDMCRKRNDYDDTIPILDAL
KVTANIVFGYATCQGAEAFEIQHSGLANGIFMKFLKDRLLEDKKITVLLDEVAEDMGKCH
LTKGKQALEIRSSLSEKRALTDPIQGTEYSAESLVRNLQWAKAHELPESMCLKFDCGVQI
QLGFAAEFSNVMIIYTSIVYKPPEIIMCDAYVTDFPLDLDIDPKDANKGTPEETGSYLVS
KDLPKHCLYTRLSSLQKLKEHLVFTVCLSYQYSGLEDTVEDKQEVNVGKPLIAKLDMHRG
LGRKTCFQTCLMSNGPYQSSAATSGGAGHYHSLQDPFHGVYHSHPGNPSNVTPADSCHCS
RTPDAFISSFAHHASCHFSRSNVPVETTDEIPFSFSDRLRISEK

Non-limiting examples of dysregulation of a MALT1 gene or a MALT1 protein are shown in Table B1 below.

TABLE B1
MALT1 Protein Amino Acid Substitutions/Insertions/Deletions

| Amino Acid | Non-limiting Exemplary | Non-Limiting Exemplary |
|---|---|---|
| Position(s) | Mutations | MALT1-associated Cancers |
| 717 | M717I⁴ | |

MALT Fusion Partners

| | Non-limiting Exemplary MALT1- |
|---|---|
| Fusion Partner | Associated Cancer(s) |
| BIRC3 (Also called IAP2; | Diffuse Large B-cell Lymphoma (DLBCL)¹: |
| CIAP2; and API2)¹ | Extra nodal low-grade MALT lymphoma² |
| IGH | ABC DLBCL² |
| SEC11C | Breast Cancer³ |

¹U.S. Pat. No. 10,711,036
²United States Patent Application Publication US20190160045A1
³United States Patent Application Publication US20130096021A1
⁴United States Patent Application Publication US20150320754A1

The term “CARD11-associated autoimmune disorder” as used herein refers to autoimmune disorders associated with or having a dysregulation of a CARD11 gene, a CARD11 protein, or the expression or activity or level of any (e.g., one or more) of the same (e.g., any of the types of dysregulation of a CARD11 gene, a CARD11 protein, or the expression or activity or level of any of the same described herein).

The term “CARD11-associated inflammatory disorder” as used herein refers to inflammatory disorders associated with or having a dysregulation of a CARD11 gene, a CARD11 protein, or the expression or activity or level of any (e.g., one or more) of the same (e.g., any of the types of dysregulation of a CARD11 gene. a CARD11 protein, or the expression or activity or level of any of the same described herein).

The term “CARD11-associated cancer” as used herein refers to cancers associated with or having a dysregulation of a CARD11 gene, a CARD11 protein, or the expression or activity or level of any (e.g., one or more) of the same (e.g., any of the types of dysregulation of a CARD11 gene, a CARD11 protein, or the expression or activity or level of any of the same described herein). Non-limiting examples of a CARD11-associated cancer are described herein.

The phrase “dysregulation of a CARD11 gene, a CARD11 protein, or the expression or activity or level of any of the same” refers to a genetic mutation (e.g., a chromosomal translocation that results in the expression of a fusion protein including a CARD11 domain and a fusion partner, a mutation in a CARD11 gene that results in the expression of a CARD11 protein that includes a deletion of at least one amino acid as compared to a wild-type CARD11 protein, a mutation in a CARD11 gene that results in the expression of a CARD11 protein with one or more point mutations as compared to a wild-type CARD11 protein, a mutation in a CARD11 gene that results in the expression of a CARD11 protein with at least one inserted amino acid as compared to a wild-type CARD11 protein, a gene duplication that results in an increased level of CARD11 protein in a cell, or a mutation in a regulatory sequence (e.g., a promoter and/or enhancer) that results in an increased level of CARD11 protein in a cell), an alternative spliced version of a CARD11 mRNA that results in a CARD11 protein having a deletion of at least one amino acid in the CARD11 protein as compared to the wild-type CARD11 protein, or increased expression (e.g., increased levels) of a wild-type CARD11 protein in a mammalian cell due to aberrant cell signaling and/or dysregulated autocrine/paracrine signaling (e.g., as compared to a control non-cancerous cell). As another example, a dysregulation of a CARD11 gene, a CARD11 protein, or expression or activity, or level of any of the same, can be a mutation in a CARD11 gene that encodes a CARD11 protein that is constitutively active or has increased activity as compared to a protein encoded by a CARD11 gene that does not include the mutation. As a further example, an increased copy number of the CARD11 gene can result in overexpression of CARD11 protein. For example, a dysregulation of a CARD11 gene, a CARD11 protein, or expression or activity, or level of any of the same, can be the result of a gene or chromosome translocation which results in the expression of a fusion protein that contains a first portion of CARD11, and a second portion of a partner protein (i.e., that is not CARD11). In some examples, dysregulation of a CARD11 gene, a CARD11 protein, or expression or activity or level of any of the same can be a result of a gene translocation of one CARD11 gene with another non-CARD11 gene.

An exemplary sequence of human CARD11 is shown below:

(UniParc Accession No. UPI00003FED38)
SEQ ID NO: 8
MPGGGPEMDDYMETLKDEEDALWENVECNRHMLSRYINPAKLTPYLRQCKVIDEQDEDEV
LNAPMLPSKINRAGRLLDILHTKGQRGYVVFLESLEFYYPELYKLVTGKEPTRRFSTIVV
EEGHEGLTHELMNEVIKLQQQMKAKDLQRCELLARLRQLEDEKKQMTLTRVELLTFQERY
YKMKEERDSYNDELVKVKDDNYNLAMRYAQLSEEKNMAVMRSRDLQLEIDQLKHRLNKME
EECKLERNQSLKLKNDIENRPKKEQVLELERENEMLKTKNQELQSIIQAGKRSLPDSDKA
ILDILEHDRKEALEDRQELVNRIYNLQEEARQAEELRDKYLEEKEDLELKCSTLGKDCEM
YKHRMNTVMLQLEEVERERDQAFHSRDEAQTQYSQCLIEKDKYRKQIRELEEKNDEMRIE
MVRREACIVNLESKLRRLSKDSNNLDQSLPRNLPVTIISQDFGDASPRINGQEADDSSTS
EESPEDSKYFLPYHPPQRRMNLKGIQLQRAKSPISLKRTSDFQAKGHEEEGTDASPSSCG
SLPITNSFTKMQPPRSRSSIMSITAEPPGNDSIVRRYKEDAPHRSTVEEDNDSGGFDALD
LDDDSHERYSFGPSSIHSSSSSHQSEGLDAYDLEQVNLMERKESLERPFRPSVTSVGHVR
GPGPSVQHTTLNGDSLTSQLTLLGGNARGSFVHSVKPGSLAEKAGLREGHQLLLLEGCIR
GERQSVPLDTCTKEEAHWTIQRCSGPVTLHYKVNHEGYRKLVKDMEDGLITSGDSFYIRL
NLNISSQLDACTMSLKCDDVVHVRDTMYQDRHEWLCARVDPFTDHDLDMGTIPSYSRAQQ
LLLVKLQRLMHRGSREEVDGTHHTLRALRNTLQPEEALSTSDPRVSPRLSRASELFGQLL
QFVSRSENKYKRMNSNERVRIISGSPLGSLARSSLDATKLLTEKQEELDPESELGKNLSL
IPYSLVRAFYCERRRPVLFTPTVLAKTLVQRLLNSGGAMEFTICKSDIVTRDEFLRRQKT
ETIIYSREKNPNAFECIAPANIEAVAAKNKHCLLEAGIGCTRDLIKSNIYPIVLFIRVCE
KNIKRFRKLLPRPETEEEFLRVCRLKEKELEALPCLYATVEPDMWGSVEELLRVVKDKIG
EEQRKTIWVDEDQL

Non-limiting examples of dysregulation of a CARD11 gene or a CARD11 protein are shown in Table B2 below.

TABLE B2
CARD11 Protein Amino Acid Substitutions/Insertions/Deletions

| Amino Acid | Non-limiting Exemplary | Non-Limiting Exemplary |
|---|---|---|
| Position(s) | Mutations | CARD11-associated Cancers |
| 47 | R47C² | Cutaneous squamous |
| | | cell carcinoma² |
| 123 | G123S¹ | Lymphoma¹ |
| 126 | G126D¹ | Lymphoma¹ |
| 130 | F130V² | Cutaneous squamous |
| | | cell carcinoma² |
| 167 | T167M² | Cutaneous squamous |
| | | cell carcinoma² |
| 215 | K215M, K215N¹ | Lymphoma¹ |
| 230 | D230N¹ | Lymphoma¹ |
| 357 | D357E¹ | Lymphoma¹ |
| 360 | M360V¹ | Lymphoma¹ |
| 361 | Y361C¹ | Lymphoma¹ |
| 368 | V368I² | Cutaneous squamous |
| | | cell carcinoma² |
| 737 | H737L² | Cutaneous squamous |
| | | cell carcinoma² |
| 750 | H750R² | Cutaneous squamous |
| | | cell carcinoma² |
| 833 | P833L² | Cutaneous squamous |
| | | cell carcinoma² |
| 900 | L900F² | Cutaneous squamous |
| | | cell carcinoma² |
| 1015 | L1015F² | Cutaneous squamous |
| | | cell carcinoma² |
| 1016 | R1016L² | Cutaneous squamous |
| | | cell carcinoma² |
| 1085 | R1085S² | Cutaneous squamous |
| | | cell carcinoma² |
| 1086 | F1086S² | Cutaneous squamous |
| | | cell carcinoma² |

¹Wu, et al., Oncotarget 7.25 (2016): 38180.
²Watt, et al. The American Journal of Pathology 185.9 (2015): 2354-2363.

The term “CARD14-associated autoimmune disorder” as used herein refers to autoimmune disorders associated with or having a dysregulation of a CARD14 gene, a CARD14 protein, or the expression or activity or level of any (e.g., one or more) of the same (e.g., any of the types of dysregulation of a CARD14 gene, a CARD14 protein, or the expression or activity or level of any of the same described herein).

The term “CARD14-associated inflammatory disorder” as used herein refers to inflammatory disorders associated with or having a dysregulation of a CARD14 gene, a CARD14 protein, or the expression or activity or level of any (e.g., one or more) of the same (e.g., any of the types of dysregulation of a CARD14 gene, a CARD14 protein, or the expression or activity or level of any of the same described herein).

The term “CARD14-associated cancer” as used herein refers to cancers associated with or having a dysregulation of a CARD14 gene, a CARD14 protein, or the expression or activity or level of any (e.g., one or more) of the same (e.g., any of the types of dysregulation of a CARD14 gene, a CARD14 protein, or the expression or activity or level of any of the same described herein).

An exemplary sequence of human CARD14 is shown below:

(UniParc Accession No. UPI000013D81B)
SEQ ID NO: 9
MGELCRRDSALTALDEETLWEMMESHRHRIVRCICPSRLTPYLRQAKVLCQLDEEEVLHS
PRLTNSAMRAGHLLDLLKTRGKNGAIAFLESLKFHNPDVYTLVTGLQPDVDFSNFSGLME
TSKLTECLAGAIGSLQEELNQEKGQKEVLLRRCQQLQEHLGLAETRAEGLHQLEADHSRM
KREVSAHFHEVLRLKDEMLSLSLHYSNALQEKELAASRCRSLQEELYLLKQELQRANMVS
SCELELQEQSLRTASDQESGDEELNRLKEENEKLRSLTESLAEKDILEQSLDEARGSRQE
LVERIHSLRERAVAAERQREQYWEEKEQTLLQFQKSKMACQLYREKVNALQAQVCELQKE
RDQAYSARDSAQREISQSLVEKDSLRRQVFELTDQVCELRTQLRQLQAEPPGVLKQEART
REPCPREKQRLVRMHAICPRDDSDCSLVSSTESQLLSDLSATSSRELVDSFRSSSPAPPS
QQSLYKRVAEDFGEEPWSFSSCLEIPEGDPGALPGAKAGDPHLDYELLDTADLPQLESSL
QPVSPGRLDVSESGVLMRRRPARRILSQVTMLAFQGDALLEQISVIGGNLTGIFIHRVTP
GSAADQMALRPGTQIVMVDYEASEPLFKAVLEDTTLEEAVGLLRRVDGFCCLSVKVNTDG
YKRLLQDLEAKVATSGDSFYIRVNLAMEGRAKGELQVHCNEVLHVTDTMFQGCGCWHAHR
VNSYTMKDTAAHGTIPNYSRAQQQLIALIQDMTQQCTVTRKPSSGGPQKLVRIVSMDKAK
ASPLRLSFDRGQLDPSRMEGSSTCFWAESCLTLVPYTLVRPHRPARPRPVLLVPRAVGKI
LSEKLCLLQGFKKCLAEYLSQEEYEAWSQRGDIIQEGEVSGGRCWVTRHAVESLMEKNTH
ALLDVQLDSVCTLHRMDIFPIVIHVSVNEKMAKKLKKGLQRLGTSEEQLLEAARQEEGDL
DRAPCLYSSLAPDGWSDLDGLLSCVRQAIADEQKKVVWTEQSPR

The term “CARD10-associated autoimmune disorder” as used herein refers to autoimmune disorders associated with or having a dysregulation of a CARD10 gene, a CARD10 protein, or the expression or activity or level of any (e.g., one or more) of the same (e.g., any of the types of dysregulation of a CARD10 gene, a CARD10 protein, or the expression or activity or level of any of the same described herein).

The term “CARD10-associated inflammatory disorder” as used herein refers to inflammatory disorders associated with or having a dysregulation of a CARD10 gene, a CARD10 protein, or the expression or activity or level of any (e.g., one or more) of the same (e.g., any of the types of dysregulation of a CARD10 gene, a CARD10 protein, or the expression or activity or level of any of the same described herein).

The term “CARD10-associated cancer” as used herein refers to cancers associated with or having a dysregulation of a CARD10 gene, a CARD10 protein, or the expression or activity or level of any (e.g., one or more) of the same (e.g., any of the types of dysregulation of a CARD10 gene, a CARD10 protein, or the expression or activity or level of any of the same described herein).

The phrase “dysregulation of a CARD10 gene, a CARD10 protein, or the expression or activity or level of any of the same” refers to a genetic mutation (e.g., a chromosomal translocation that results in the expression of a fusion protein including a CARD10 domain and a fusion partner, a mutation in a CARD10 gene that results in the expression of a CARD10 protein that includes a deletion of at least one amino acid as compared to a wild-type CARD10 protein, a mutation in a CARD10 gene that results in the expression of a CARD10 protein with one or more point mutations as compared to a wild-type CARD10 protein, a mutation in a CARD10 gene that results in the expression of a CARD10 protein with at least one inserted amino acid as compared to a wild-type CARD10 protein, a gene duplication that results in an increased level of CARD10 protein in a cell, or a mutation in a regulatory sequence (e.g., a promoter and/or enhancer) that results in an increased level of CARD10 protein in a cell), an alternative spliced version of a CARD10 mRNA that results in a CARD10 protein having a deletion of at least one amino acid in the CARD10 protein as compared to the wild-type CARD10 protein, or increased expression (e.g., increased levels) of a wild-type CARD10 protein in a mammalian cell due to aberrant cell signaling and/or dysregulated autocrine/paracrine signaling (e.g., as compared to a control non-cancerous cell). As another example, a dysregulation of a CARD10 gene, a CARD10 protein, or expression or activity, or level of any of the same, can be a mutation in a CARD10 gene that encodes a CARD10 protein that is constitutively active or has increased activity as compared to a protein encoded by a CARD10 gene that does not include the mutation. As a further example, an increased copy number of the CARD10 gene can result in overexpression of CARD10 protein. For example, a dysregulation of a CARD10 gene, a CARD10 protein, or expression or activity, or level of any of the same, can be the result of a gene or chromosome translocation which results in the expression of a fusion protein that contains a first portion of CARD10, and a second portion of a partner protein (i.e., that is not CARD10). In some examples, dysregulation of a CARD10 gene, a CARD10 protein, or expression or activity or level of any of the same can be a result of a gene translocation of one CARD10 gene with another non-CARD10 gene.

An exemplary sequence of human CARD10 is shown below:

(UniParc Accession No. UPI0000044645)
SEQ ID NO: 10
MPGRAEAGEAEEEAGAGSGSEAEEDALWERIEGVRHRLARALNPAKLTPYLRQCRVIDEQ
DEEEVLSTYRFPCRVNRTGRLMDILRCRGKRGYEAFLEALEFYYPEHFTLLIGQEPAQRC
SMILDEEGPEGLTQFLMTEVRRLREARKSQLQREQQLQARGRVLEEERAGLEQRLRDQQQ
AQERCQRLREDWEAGSLELLRLKDENYMIAMRLAQLSEEKNSAVLRSRDLQLAVDQLKLK
VSRLEEECALLRRARGPPPGAEEKEKEKEKEKEPDNVDLVSELRAENQRLTASLRELQEG
LQQEASRPGAPGSERILLDILEHDWREAQDSRQELCQKLHAVQGELQWAEELRDQYLQEM
EDLRLKHRTLQKDCDLYKHRMATVLAQLEEIEKERDQAIQSRDRIQLQYSQSLIEKDQYR
KQVRGLEAERDELLITLTSLEGTKALLEVQLQRAQGGTCLKACASSHSLCSNLSSTWSLS
EFPSPLGGPEATGEAAVMGGPEPHNSEEATDSEKEINRLSILPFPPSAGSILRRQREEDP
APPKRSFSSMSDITGSVTLKPWSPGLSSSSSSDSVWPLGKPEGLLARGCGLDFLNRSLAI
RVSGRSPPGGPEPQDKGPDGLSFYGDRWSGAVVRRVLSGPGSARMEPREQRVEAAGLEGA
CLEAEAQQRTLLWNQGSTLPSLMDSKACQSFHEALEAWAKGPGAEPFYIRANLTLPERAD
PHALCVKAQEILRLVDSAYKRRQEWFCTRVDPLTLRDLDRGTVPNYQRAQQLLEVQEKCL
PSSRHRGPRSNLKKRALDQLRLVRPKPVGAPAGDSPDQLLLEPCAEPERSLRPYSLVRPL
LVSALRPVVLLPECLAPRLIRNLLDLPSSRLDFQVCPAESLSGEELCPSSAPGAPKAQPA
TPGLGSRIRAIQESVGKKHCLLELGARGVRELVQNEIYPIVIHVEVTEKNVREVRGLLGR
PGWRDSELLRQCRGSEQVLWGLPCSWVQVPAHEWGHAEELAKVVRGRILQEQARLVWVEC
GSSRGCPSSSEA

The term “CARD9-associated autoimmune disorder” as used herein refers to autoimmune disorders associated with or having a dysregulation of a CARD9 gene, a CARD9 protein, or the expression or activity or level of any (e.g., one or more) of the same (e.g., any of the types of dysregulation of a CARD9 gene, a CARD9 protein, or the expression or activity or level of any of the same described herein).

The term “CARD9-associated inflammatory disorder” as used herein refers to inflammatory disorders associated with or having a dysregulation of a CARD9 gene, a CARD9 protein, or the expression or activity or level of any (e.g., one or more) of the same (e.g., any of the types of dysregulation of a CARD9 gene, a CARD9 protein, or the expression or activity or level of any of the same described herein).

The term “CARD9-associated cancer” as used herein refers to cancers associated with or having a dysregulation of a CARD9 gene, a CARD9 protein, or the expression or activity or level of any (e.g., one or more) of the same (e.g., any of the types of dysregulation of a CARD9 gene, a CARD9 protein, or the expression or activity or level of any of the same described herein).

The phrase “dysregulation of a CARD9 gene, a CARD9 protein, or the expression or activity or level of any of the same” refers to a genetic mutation (e.g., a chromosomal translocation that results in the expression of a fusion protein including a CARD9 domain and a fusion partner, a mutation in a CARD9 gene that results in the expression of a CARD9 protein that includes a deletion of at least one amino acid as compared to a wild-type CARD9 protein, a mutation in a CARD9 gene that results in the expression of a CARD9 protein with one or more point mutations as compared to a wild-type CARD9 protein, a mutation in a CARD9 gene that results in the expression of a CARD9 protein with at least one inserted amino acid as compared to a wild-type CARD9 protein, a gene duplication that results in an increased level of CARD9 protein in a cell, or a mutation in a regulatory sequence (e.g., a promoter and/or enhancer) that results in an increased level of CARD9 protein in a cell), an alternative spliced version of a CARD9 mRNA that results in a CARD9 protein having a deletion of at least one amino acid in the CARD9 protein as compared to the wild-type CARD9 protein, or increased expression (e.g., increased levels) of a wild-type CARD9 protein in a mammalian cell due to aberrant cell signaling and/or dysregulated autocrine/paracrine signaling (e.g., as compared to a control non-cancerous cell). As another example, a dysregulation of a CARD9 gene, a CARD9 protein, or expression or activity, or level of any of the same, can be a mutation in a CARD9 gene that encodes a CARD9 protein that is constitutively active or has increased activity as compared to a protein encoded by a CARD9 gene that does not include the mutation. As a further example, an increased copy number of the CARD9 gene can result in overexpression of CARD9 protein. For example, a dysregulation of a CARD9 gene, a CARD9 protein, or expression or activity, or level of any of the same, can be the result of a gene or chromosome translocation which results in the expression of a fusion protein that contains a first portion of CARD9, and a second portion of a partner protein (i.e., that is not CARD9). In some examples, dysregulation of a CARD9 gene, a CARD9 protein, or expression or activity or level of any of the same can be a result of a gene translocation of one CARD9 gene with another non-CARD9 gene.

An exemplary sequence of human CARD9 is shown below:

(UniParc Accession No. UPI000013E4EB)
SEQ ID NO: 11
MSDYENDDECWSVLEGERVILTSVIDPSRITPYLRQCKVLNPDDEEQVLSDPNLVIRKRK
VGVLLDILQRIGHKGYVAFLESLELYYPQLYKKVTGKEPARVFSMIIDASGESGLTQLLM
TEVMKLQKKVQDLTALLSSKDDFIKELRVKDSLLRKHQERVQRLKEECEAGSRELKRCKE
ENYDLAMRLAHQSEEKGAALMRNRDLQLEIDQLKHSLMKAEDDCKVERKHTLKLRHAMEQ
RPSQELLWELQQEKALLQARVQELEASVQEGKLDRSSPYIQVLEEDWRQALRDHQEQANT
IFSLRKDLRQGEARRLRCMEEKEMFELQCLALRKDSKMYKDRIEAILLQMEEVAIERDQA
IATREELHAQHARGLQEKDALRKQVRELGEKADELQLQVFQCEAQLLAVEGRLRRQQLET
LVLSSDLEDGSPRRSQELSLPQDLEDTQLSDKGCLAGGGSPKQPFAALHQEQVLRNPHDA
GLSSGEPPEKERRRLKESFENYRRKRALRKMQKGWRQGEEDRENTTGSDNTDTEGS

The term “BCL10-associated autoimmune disorder” as used herein refers to autoimmune disorders associated with or having a dysregulation of a BCL10 gene, a BCL10 protein, or the expression or activity or level of any (e.g., one or more) of the same (e.g., any of the types of dysregulation of a BCL10 gene, a BCL10 protein, or the expression or activity or level of any of the same described herein).

The term “BCL10-associated inflammatory disorder” as used herein refers to inflammatory disorders associated with or having a dysregulation of a BCL10 gene, a BCL10 protein, or the expression or activity or level of any (e.g., one or more) of the same (e.g., any of the types of dysregulation of a BCL10 gene, a BCL10 protein, or the expression or activity or level of any of the same described herein).

The term “BCL10-associated cancer” as used herein refers to cancers associated with or having a dysregulation of a BCL10 gene, a BCL10 protein, or the expression or activity or level of any (e.g., one or more) of the same (e.g., any of the types of dysregulation of a BCL10 gene, a BCL10 protein, or the expression or activity or level of any of the same described herein).

The phrase “dysregulation of a BCL10 gene, a BCL10 protein, or the expression or activity or level of any of the same” refers to a genetic mutation (e.g., a chromosomal translocation that results in the expression of a fusion protein including a BCL10 domain and a fusion partner, a mutation in a BCL10 gene that results in the expression of a BCL10 protein that includes a deletion of at least one amino acid as compared to a wild-type BCL10 protein, a mutation in a BCL10 gene that results in the expression of a BCL10 protein with one or more point mutations as compared to a wild-type BCL10 protein, a mutation in a BCL10 gene that results in the expression of a BCL10 protein with at least one inserted amino acid as compared to a wild-type BCL10 protein, a gene duplication that results in an increased level of BCL10 protein in a cell, or a mutation in a regulatory sequence (e.g., a promoter and/or enhancer) that results in an increased level of BCL10 protein in a cell), an alternative spliced version of a BCL10 mRNA that results in a BCL10 protein having a deletion of at least one amino acid in the BCL10 protein as compared to the wild-type BCL10 protein, or increased expression (e.g., increased levels) of a wild-type BCL10 protein in a mammalian cell due to aberrant cell signaling and/or dysregulated autocrine/paracrine signaling (e.g., as compared to a control non-cancerous cell). For example, a dysregulation of a BCL10 gene, a BCL10 protein, or expression or activity, or level of any of the same, can be the result of a gene or chromosome translocation which results in the expression of a fusion protein that contains a first portion of BCL10, and a second portion of a partner protein (i.e., that is not BCL10). In some examples, dysregulation of a BCL10 gene, a BCL10 protein, or expression or activity or level of any of the same can be a result of a gene translocation of one BCL10 gene with another non-BCL10 gene.

An exemplary sequence of human BCL10 is shown below:

(UniParc Accession No. UPI000012682F)
SEQ ID NO: 12
MEPTAPSLTEEDLTEVKKDALENLRVYLCEKIIAERHFDHLRAKKILSREDTEEISCRTS
SRKRAGKLLDYLQENPKGLDTLVESIRREKTQNFLIQKITDEVLKLRNIKLEHLKGLKCS
SCEPFPDGATNNLSRSNSDESNFSEKLRASTVMYHPEGESSTTPFFSTNSSLNLPVLEVG
RTENTIFSSTILPRPGDPGAPPLPPDLQLEEEGTCANSSEMFLPLRSRTVSRQ

Non-limiting examples of dysregulation of a BCL10 gene or a BCL10 protein are shown in Table B3 below.

TABLE B3
BCL10 Protein Amino Acid Substitutions/Insertions/Deletions

| Amino Acid | Non-limiting Exemplary | Non-Limiting Exemplary |
|---|---|---|
| Position(s) | Mutations | BCL10-associated Cancers |
| 5 | A5S² | Lymphoma² |
| 16 | V16E² | Lymphoma² |
| 20 | A20T¹ | Germ cell tumor¹ |
| 31 | K31E | Lymphoma² |
| 32 | I32V¹ | Lymphoma¹ |
| 13 | A13*² | Lymphoma² |
| 46 | I46*¹ | T-ALL¹, colonic carcinoma¹ |
| 49 | R49G¹ | Lymphoma¹ |
| 52 | T52I¹ | Mesothelioma¹ |
| 55 | I55*¹ | Lymphoma¹ |
| 57 | C57R² | Lymphoma² |
| 58 | R58G¹, R58*¹ | Germ cell tumor¹ |
| 64 | R64K² | Lymphoma² |
| 77 | K77*¹ | Lymphoma¹ |
| 80 | D80N | Lymphoma¹ |
| 91 | T91*¹ | Germ cell tumor¹ |
| 100 | T100S¹ | Lymphoma¹ |
| 101 | D101E² | Lymphoma² |
| 115 | K115*¹ | Lymphoma¹ |
| 116-126 | Splice mutation¹ | Lymphoma¹ |
| 116-121 | Splice mutation² | Lymphoma² |
| 116-120 | Splice mutation¹ | Mesothelioma¹ |
| 133 | L133*¹ | Lymphoma¹ |
| 134 | S134P² | Lymphoma² |
| 137 | N137*¹ | Lymphoma¹ |
| 143 | F143*¹ | Lymphoma¹ |
| 152 | V152*² | Lymphoma² |
| 165 | F165*² | Lymphoma² |
| 167 | S167*¹ | Lymphoma¹ |
| 168 | T168A² | Lymphoma² |
| 170-180 | del S170-G180¹ | Lymphoma¹ |
| 175-181 | del P175-G180¹ | Lymphoma¹ |
| 210 | del 210¹ | Lymphoma¹ |
| 213 | G213E | Lymphoma² |
| 218 | S218F¹ | Germ cell tumor¹ |
| 230 | V230I² | Lymphoma² |
| Stop | Stop->R | Lymphoma² |

¹Willis, et al. Cell 96.1 (1999): 35-45.
²Zhang, et al. Nature Genetics 22.1 (1999): 63-68.

The term “MALT1 protease substrate-associated cancer” as used herein refers to cancers associated with or having a dysregulation of a gene, a protein, or the expression or activity or level of any (e.g., one or more) of the same associated with a MALT1 protease substrate. In some embodiments, a MALT1 protease substrate-associated cancer is selected from the group consisting of a BCL10-associated cancer, an A20-associated cancer, a CYLD-associated cancer, a RelB-associated cancer, a Regnase 1-associated cancer, a roquin-1-associated cancer, a HOIL1-associated cancer, a NIK associated cancer, a LIMA1α-associated cancer, and combinations thereof. In some embodiments, a MALT1 protease substrate-associated cancer is selected from the group consisting of a BCL10-associated cancer, an A20-associated cancer, a CYLD-associated cancer, and combinations thereof. The cancers “associated” with a particular gene or protein described in this paragraph refer to cancers associated with or having a dysregulation of the particular gene, the particular protein, or the expression or activity or level of any (e.g., one or more) of the same (e.g., any of the types of dysregulation of the particular gene, the particular protein, or the expression or activity or level of any of the same described herein). Non-limiting examples of such cancers are described herein.

An exemplary sequence of human A20 is shown below:

(UniParc Accession No. UPI000000D92D)
SEQ ID NO: 13
MAEQVLPQALYLSNMRKAVKIRERTPEDIFKPTNGIIHHFKTMHRYTLEMERTCQFCPQF
REIIHKALIDRNIQATLESQKKLNWCREVRKLVALKINGDGNCLMHATSQYMWGVQDTDL
VLRKALFSTLKETDTRNFKFRWQLESLKSQEFVETGLCYDTRNWNDEWDNLIKMASTDTP
MARSGLQYNSLEEIHIFVLCNILRRPIIVISDKMLRSLESGSNFAPLKVGGIYLPLHWPA
QECYRYPIVLGYDSHHFVPLVTLKDSGPEIRAVPLVNRDRGRFEDLKVHFLIDPENEMKE
KLLKEYLMVIEIPVQGWDHGTTHLINAAKLDEANLPKEINLVDDYFELVQHEYKKWQENS
EQGRREGHAQNPMEPSVPQLSLMDVKCETPNCPFFMSVNTQPLCHECSERRQKNQNKLPK
LNSKPGPEGLPGMALGASRGEAYEPLAWNPEESTGGPHSAPPTAPSPELFSETTAMKCRS
PGCPFTLNVQHNGFCERCHNARQLHASHAPDHTRHLDPGKCQACLQDVTRTENGICSTCF
KRITAEASSSLSTSLPPSCHQRSKSDPSRLVRSPSPHSCHRAGNDAPAGCLSQAARTPGD
RTGTSKCRKAGCVYFGTPENKGFCTLCFIEYRENKHFAAASGKVSPTASRFQNTIPCLGR
ECGTLGSTMFEGYCQKCFIEAQNQRFHEAKRTEEQLRSSQRRDVPRTTQSTSRPKCARAS
CKNILACRSEELCMECQHPNQRMGPGAHRGEPAPEDPPKQRCRAPACDHFGNAKCNGYCN
ECFQFKQMYG

Non-limiting examples of dysregulation of an A20 gene or an A20 protein are shown in Table B4 below.

TABLE B4
A20 Protein Amino Acid Substitutions/Insertions/Deletions

| Amino Acid | Non-limiting Exemplary | Non-Limiting Exemplary |
|---|---|---|
| Position(s) | Mutations | A20-associated Cancers |
| 100 | D100*² | Extranodal marginal zone |
| | | lymphoma² |
| 162 | R162*² | Nodal marginal zone |
| | | lymphoma² |
| 183 | R183X¹ | Lymphoma¹ |
| 271 | R271X¹ | Lymphoma¹ |
| 278 | R278*² | Nodal marginal zone |
| | | lymphoma² |
| 288 | V288*² | Splenic marginal zone |
| | | lymphoma² |
| 491 | H491*² | Nodal marginal zone |
| | | lymphoma² |
| 633 | E633*² | Extranodal marginal zone |
| | | lymphoma² |

¹Johansson et al. Oncotarget 7.38 (2016): 62627.
²Novak, et al. Blood 113.20 (2009): 4918-4921.

An exemplary sequence of human CYLD is shown below:

(UniParc Accession No. UPI0000073A15)
SEQ ID NO: 14
MSSGLWSQEKVTSPYWEERIFYLLLQECSVTDKQTQKLLKVPKGSIGQYIQDRSVGHSRI
PSAKGKKNQIGLKILEQPHAVLFVDEKDVVEINEKFTELLLAITNCEERFSLFKNRNRLS
KGLQIDVGCPVKVQLRSGEEKFPGVVRFRGPLLAERTVSGIFFGVELLEEGRGQGFTDGV
YQGKQLFQCDEDCGVFVALDKLELIEDDDTALESDYAGPGDTMQVELPPLEINSRVSLKV
GETIESGTVIFCDVLPGKESLGYFVGVDMDNPIGNWDGRFDGVQLCSFACVESTILLHIN
DIIPALSESVTQERRPPKLAFMSRGVGDKGSSSHNKPKATGSTSDPGNRNRSELFYTLNG
SSVDSQPQSKSKNTWYIDEVAEDPAKSLTEISTDFDRSSPPLQPPPVNSLITENRFHSLP
FSLTKMPNINGSIGHSPLSLSAQSVMEELNTAPVQESPPLAMPPGNSHGLEVGSLAEVKE
NPPFYGVIRWIGQPPGLNEVLAGLELEDECAGCTDGTFRGTRYFTCALKKALFVKLKSCR
PDSRFASLQPVSNQIERCNSLAFGGYLSEVVEENTPPKMEKEGLEIMIGKKKGIQGHYNS
CYLDSTLFCLFAFSSVLDTVLLRPKEKNDVEYYSETQELLRTEIVNPLRIYGYVCATKIM
KLRKILEKVEAASGFTSEEKDPEEFLNILFHHILRVEPLLKIRSAGQKVQDCYFYQIFME
KNEKVGVPTIQQLLEWSFINSNLKFAEAPSCLIIQMPRFGKDFKLFKKIFPSLELNITDL
LEDTPRQCRICGGLAMYECRECYDDPDISAGKIKQFCKTCNTQVHLHPKRLNHKYNPVSL
PKDLPDWDWRHGCIPCQNMELFAVLCIETSHYVAFVKYGKDDSAWLFFDSMADRDGGQNG
FNIPQVTPCPEVGEYLKMSLEDLHSLDSRRIQGCARRLLCDAYMCMYQSPTMSLYK

Non-limiting examples of dysregulation of a CYLD gene or a CYLD protein can be found, for example, in Massoumi, Future Oncology 7.2 (2011): 285-297, Alameda, J. P., et al., Oncogene 29.50 (2010): 6522-6532, Williams, et al., Modern Pathology (2020): 1-13, and Courtois and Gilmore. Oncogene 25.51 (2006): 6831-6843.

An exemplary sequence of human RelB is shown below:

(UniParc Accession No. UPI00000012B7)
SEQ ID NO: 15
MLRSGPASGPSVPTGRAMPSRRVARPPAAPELGALGSPDLSSLSLAVSRSTDELEIIDEY
IKENGFGLDGGQPGPGEGLPRLVSRGAASLSTVTLGPVAPPATPPPWGCPLGRLVSPAPG
PGPQPHLVITEQPKQRGMRFRYECEGRSAGSILGESSTEASKILPAIELRDCGGLREVEV
TACLVWKDWPHRVHPHSLVGKDCTDGICRVRLRPHVSPRHSFNNLGIQCVRKKEIEAAIE
RKIQLGIDPYNAGSLKNHQEVDMNVVRICFQASYRDQQGQMRRMDPVLSEPVYDKKSINT
SELRICRINKESGPCTGGEELYLLCDKVQKEDISVVFSRASWEGRADFSQADVHRQIAIV
FKTPPYEDLEIVEPVTVNVFLQRLTDGVCSEPLPFTYLPRDHDSYGVDKKRKRGMPDVLG
ELNSSDPHGIESKRRKKKPAILDHELPNHGSGPFLPPSALLPDPDFFSGTVSLPGLEPPG
GPDLLDDGFAYDPTAPTLFTMLDLLPPAPPHASAVVCSGGAGAVVGETPGPEPLTLDSYQ
APGPGDGGTASLVGSNMFPNHYREAAFGGGLLSPGPEAT

An exemplary sequence of human Regnase 1 is shown below:

(UniParc Accession No. UPI000004D30E)
SEQ ID NO: 16
MSGPCGEKPVLEASPTMSLWEFEDSHSRQGTPRPGQELAAEEASALELQMKVDFFRKLGY
SSTEIHSVLQKLGVQADTNTVLGELVKHGTATERERQTSPDPCPQLPLVPRGGGTPKAPN
LEPPLPEEEKEGSDLRPVVIDGSNVAMSHGNKEVFSCRGILLAVNWFLERGHTDITVFVP
SWRKEQPRPDVPITDQHILRELEKKKILVFTPSRRVGGKRVVCYDDRFIVKLAYESDGIV
VSNDTYRDLQGERQEWKRFIEERLLMYSFVNDKFMPPDDPLGRHGPSLDNFLRKKPLTLE
HRKQPCPYGRKCTYGIKCRFFHPERPSCPQRSVADELRANALLSPPRAPSKDKNGRRPSP
SSQSSSLLTESEQCSLDGKKLGAQASPGSRQEGLTQTYAPSGRSLAPSGGSGSSFGPTDW
LPQTLDSLPYVSQDCLDSGIGSLESQMSELWGVRGGGPGEPGPPRAPYTGYSPYGSELPA
TAAFSAFGRAMGAGHFSVPADYPPAPPAFPPREYWSEPYPLPPPTSVLQEPPVQSPGAGR
SPWGRAGSLAKEQASVYTKLCGVFPPHLVEAVMGRFPQLLDPQQLAAEILSYKSQHPSE

An exemplary sequence of human roquin-1 is shown below:

(UniParc Accession No. UPI00001D7DA8)
SEQ ID NO: 17
MPVQAPQWTDFLSCPICTQTFDETIRKPISLGCGHTVCKMCLNKLHRKACPFDQTTINTD
IELLPVNSALLQLVGAQVPEQQPITLCSGVEDTKHYEEAKKCVEELALYLKPLSSARGVG
LNSTTQSVLSRPMQRKLVTLVHCQLVEEEGRIRAMRAARSLGERTVTELILQHQNPQQLS
SNLWAAVRARGCQFLGPAMQEEALKLVLLALEDGSALSRKVLVLFVVQRLEPREPQASKT
SIGHVVQLLYRASCFKVTKRDEDSSLMQLKEEFRTYEALRREHDSQIVQIAMEAGLRIAP
DQWSSLLYGDQSHKSHMQSIIDKLQTPASFAQSVQELTIALQRTGDPANLNRLRPHLELL
ANIDPSPDAPPPTWEQLENGLVAVRTVVHGLVDYIQNHSKKGADQQQPPQHSKYKTYMCR
DMKQRGGCPRGASCTFAHSQEELEKFRKMNKRLVPRRPLSASLGQLNEVGLPSAAILPDE
GAVDLPSRKPPALPNGIVSTGNTVTQLIPRGTDPSYDSSLKPGKIDHLSSSAPGSPPDLL
ESVPKSISALPVNPHSIPPRGPADLPPMPVTKPLQMVPRGSQLYPAQQTDVYYQDPRGAA
PPFEPAPYQQGMYYTPPPQCVSRFVRPPPSAPEPAPPYLDHYPPYLQERVVNSQYGTQPQ
QYPPIYPSHYDGRRVYPAPSYTREEIFRESPIPIEIPPAAVPSYVPESRERYQQIESYYP
VAPHPTQIRPSYLREPPYSRLPPPPQPHPSLDELHRRRKEIMAQLEERKVISPPPFAPSP
TLPPTFHPEEFLDEDLKVAGKYKGNDYSQYSPWSCDTIGSYIGTKDAKPKDVVAAGSVEM
MNVESKGMRDQRLDLQRRAAETSDDDLIPFGDRPTVSRFGAISRISKTIYQGAGPMQAMA
PQGAPTKSINISDYSPYGTHGGWGASPYSPHQNIPSQGHFSERERISMSEVASHGKPLPS
AEREQLRLELQQLNHQISQQTQLRGLEAVSNRLVLQREANTLAGQSQPPPPPPPKWPGMI
SSEQLSLELHQVEREIGKRTRELSMENQCSLDMKSKLNTSKQAENGQPEPQNKVPAEDLT
LTFSDVPNGSALTQENISLLSNKTSSLNLSEDPEGGGDNNDSQRSGVTPSSAP

An exemplary sequence of human HOIL1 is shown below:

(UniParc Accession No. UPI000006F045)
SEQ ID NO: 18
MDEKTKKAEEMALSLTRAVAGGDEQVAMKCAIWLAEQRVPLSVQLKPEVSPTQDIRLWVS
VEDAQMHTVTIWLTVRPDMTVASLKDMVFLDYGFPPVLQQWVIGQRLARDQETLHSHGVR
QNGDSAYLYLLSARNTSLNPQELQRERQLRMLEDLGFKDLTLQPRGPLEPGPPKPGVPQE
PGRGQPDAVPEPPPVGWQCPGCTFINKPTRPGCEMCCRARPEAYQVPASYQPDEEERARL
AGEEEALRQYQQRKQQQQEGNYLQHVQLDQRSLVLNTEPAECPVCYSVLAPGEAVVLREC
LHTFCRECLQGTIRNSQEAEVSCPFIDNTYSCSGKLLEREIKALLTPEDYQRFLDLGISI
AENRSAFSYHCKTPDCKGWCFFEDDVNEFTCPVCFHVNCLLCKAIHEQMNCKEYQEDLAL
RAQNDVAARQTTEMLKVMLQQGEAMRCPQCQIVVQKKDGCDWIRCTVCHTEICWVTKGPR
WGPGGPGDTSGGCRCRVNGIPCHPSCQNCH

An exemplary sequence of human NIK is shown below:

(UniParc Accession No. UPI0000074220)
SEQ ID NO: 19
MAVMEMACPGAPGSAVGQQKELPKAKEKTPPLGKKQSSVYKLEAVEKSPVFCGKWEILND
VITKGTAKEGSEAGPAAISIIAQAECENSQEFSPTFSERIFIAGSKQYSQSESLDQIPNN
VAHATEGKMARVCWKGKRRSKARKKRKKKSSKSLAHAGVALAKPLPRTPEQESCTIPVQE
DESPLGAPYVRNTPQFTKPLKEPGLGQLCFKQLGEGLRPALPRSELHKLISPLQCLNHVW
KLHHPQDGGPLPLPTHPFPYSRLPHPFPFHPLQPWKPHPLESFLGKLACVDSQKPLPDPH
LSKLACVDSPKPLPGPHLEPSCLSRGAHEKFSVEEYLVHALQGSVSSGQAHSLTSLAKTW
AARGSRSREPSPKTEDNEGVLLTEKLKPVDYEYREEVHWATHQLRLGRGSFGEVHRMEDK
QTGFQCAVKKVRLEVFRAEELMACAGLTSPRIVPLYGAVREGPWVNIFMELLEGGSLGQL
VKEQGCLPEDRALYYLGQALEGLEYLHSRRILHGDVKADNVLLSSDGSHAALCDFGHAVC
LQPDGLGKSLLTGDYIPGTETHMAPEVVLGRSCDAKVDVWSSCCMMLHMLNGCHPWTQFF
RGPLCLKIASEPPPVREIPPSCAPLTAQAIQEGLRKEPIHRVSAAELGGKVNRALQQVGG
LKSPWRGEYKEPRHPPPNQANYHQTLHAQPRELSPRAPGPRPAEETTGRAPKLQPPLPPE
PPEPNKSPPLILSKEESGMWEPLPLSSLEPAPARNPSSPERKATVPEQELQQLEIELFLN
SLSQPFSLEEQEQILSCLSIDSLSLSDDSEKNPSKASQSSRDTLSSGVHSWSSQAEARSS
SWNMVLARGRPTDTPSYFNGVKVQIQSLNGEHLHIREFHRVKVGDIATGISSQIPAAAFS
LVTKDGQPVRYDMEVPDSGIDLQCTLAPDGSFAWSWRVKHGQLENRP

An exemplary sequence of human LIMA1α is shown below:

(UniParc Accession No. UPI000002A906)
SEQ ID NO: 20
MENCLGESRHEVEKSEISENTDASGKIEKYNVPLNRLKMMFEKGEPTQTKILRAQSRSAS
GRKISENSYSLDDLEIGPGQLSSSTEDSEKNESRRNLELPRLSETSIKDRMAKYQAAVSK
QSSSTNYTNELKASGGEIKIHKMEQKENVPPGPEVCITHQEGEKISANENSLAVRSTPAE
DDSRDSQVKSEVQQPVHPKPLSPDSRASSLSESSPPKAMKKFQAPARETCVECQKTVYPM
ERLLANQQVFHISCFRCSYCNNKLSLGTYASLHGRIYCKPHFNQLFKSKGNYDEGFGHRP
HKDLWASKNENEEILERPAQLANARETPHSPGVEDAPIAKVGVLAASMEAKASSQQEKED
KPAETKKLRIAWPPPTELGSSGSALEEGIKMSKPKWPPEDEISKPEVPEDVDLDLKKLRR
SSSLKERSRPFTVAASFQSTSVKSPKTVSPPIRKGWSMSEQSEESVGGRVAERKQVENAK
ASKKNGNVGKTTWQNKESKGETGKRSKEGHSLEMENENLVENGADSDEDDNSFLKQQSPQ
EPKSLNWSSFVDNTFAEEFTTQNQKSQDVELWEGEVVKELSVEEQIKRNRYYDEDEDEE

The term “cancer associated with a component of the NF-κB pathway downstream of a CBM complex” as used herein refers to cancers associated with or having a dysregulation of a gene, a protein, or the expression or activity or level of any (e.g., one or more) of the same associated with a component of the NF-κB pathway downstream of a CBM complex. In some embodiments, a cancer associated with a component of the NF-κB pathway downstream of a CBM complex is selected from the group consisting of a TAK1-associated cancer, a TRAF6-associated cancer, a TAB1-associated cancer, a TAB2-associated cancer, a TAB3-associated cancer, a MKK7-associated cancer, an IKKα-associated cancer, an IKKβ-associated cancer, an IKKγ-associated cancer, an IκBα-associated cancer, a p50-associated cancer, a p65 (RelA)-associated cancer, a c-Rel-associated cancer, and combinations thereof. In some embodiments, a cancer associated with a component of the NF-κB pathway downstream of a CBM complex is an IKKγ-associated cancer. The cancers “associated” with a particular gene or protein described in this paragraph refer to cancers associated with or having a dysregulation of the particular gene, the particular protein, or the expression or activity or level of any (e.g., one or more) of the same (e.g., any of the types of dysregulation of the particular gene, the particular protein, or the expression or activity or level of any of the same described herein). Non-limiting examples of such cancers are described herein.

An exemplary sequence of human TAK1 is shown below:

(UniParc Accession No. UPI000012EAD6)
SEQ ID NO: 21
MSTASAASSSSSSSAGEMIEAPSQVLNFEEIDYKEIEVEEVVGRGAFGVVCKAKWRAKDV
AIKQIESESERKAFIVELRQLSRVNHPNIVKLYGACLNPVCLVMEYAEGGSLYNVLHGAE
PLPYYTAAHAMSWCLQCSQGVAYLHSMQPKALIHRDLKPPNLLLVAGGTVLKICDFGTAC
DIQTHMTNNKGSAAWMAPEVFEGSNYSEKCDVFSWGIILWEVITRRKPFDEIGGPAFRIM
WAVHNGTRPPLIKNLPKPIESLMTRCWSKDPSQRPSMEEIVKIMTHLMRYFPGADEPLQY
PCQYSDEGQSNSATSTGSFMDIASTNTSNKSDTNMEQVPATNDTIKRLESKLLKNQAKQQ
SESGRLSLGASRGSSVESLPPTSEGKRMSADMSEIEARIAATTAYSKPKRGHRKTASFGN
ILDVPEIVISGNGQPRRRSIQDLTVTGTEPGQVSSRSSSPSVRMITTSGPTSEKPTRSHP
WTPDDSTDINGSDNSIPMAYLTLDHQLQPLAPCPNSKESMAVFEQHCKMAQEYMKVQTEI
ALLLQRKQELVAELDQDEKDQQNTSRLVQEHKKLLDENKSLSTYYQQCKKQLEVIRSQQQ
KRQGTS

An exemplary sequence of human TRAF6 is shown below:

(UniParc Accession No. UPI000000D924)
SEQ ID NO: 22
MSLLNCENSCGSSQSESDCCVAMASSCSAVTKDDSVGGTASTGNLSSSF
MEEIQGYDVEFDPPLESKYECPICLMALREAVQTPCGHRFCKACIIKSI
RDAGHKCPVDNEILLENQLFPDNFAKREILSLMVKCPNEGCLHKMELRH
LEDHQAHCEFALMDCPQCQRPFQKFHINIHILKDCPRRQVSCDNCAASM
AFEDKEIHDQNCPLANVICEYCNTILIREQMPNHYDLDCPTAPIPCTFS
TFGCHEKMQRNHLARHLQENTQSHMRMLAQAVHSLSVIPDSGYISEVRN
FQETIHQLEGRLVRQDHQIRELTAKMETQSMYVSELKRTIRTLEDKVAE
IEAQQCNGIYIWKIGNFGMHLKCQEEEKPVVIHSPGFYTGKPGYKLCMR
LHLQLPTAQRCANYISLFVHTMQGEYDSHLPWPFQGTIRLTILDQSEAP
VRQNHEEIMDAKPELLAFQRPTIPRNPKGFGYVTFMHLEALRQRTFIKD
DTLLVRCEVSTRFDMGSLRREGFQPRSTDAGV

An exemplary sequence of human TAB1 is shown below:

(UniParc Accession No. UPI0000136861)
SEQ ID NO: 23
MAAQRRSLLQSEQQPSWTDDLPLCHLSGVGSASNRSYSADGKGTESHPP
EDSWLKFRSENNCFLYGVFNGYDGNRVTNFVAQRLSAELLLGQLNAEHA
EADVRRVLLQAFDVVERSFLESIDDALAEKASLQSQLPEGVPQHQLPPQ
YQKILERLKTLEREISGGAMAVVAVLLNNKLYVANVGTNRALLCKSTVD
GLQVTQLNVDHTTENEDELFRLSQLGLDAGKIKQVGIICGQESTRRIGD
YKVKYGYTDIDLLSAAKSKPIIAEPEIHGAQPLDGVTGFLVLMSEGLYK
ALEAAHGPGQANQEIAAMIDTEFAKQTSLDAVAQAVVDRVKRIHSDTFA
SGGERARFCPRHEDMTLLVRNFGYPLGEMSQPTPSPAPAAGGRVYPVSV
PYSSAQSTSKTSVTLSLVMPSQGQMVNGAHSASTLDEATPTLTNQSPTL
TLQSTNTHTQSSSSSSDGGLFRSRPAHSLPPGEDGRVEPYVDFAEFYRL
WSVDHGEQSVVTAP

An exemplary sequence of human TAB2 is shown below:

(UniParc Accession No. UPI0000073C75)
SEQ ID NO: 24
MAQGSHQIDFQVLHDLRQKFPEVPEVVVSRCMLQNNNNLDACCAVLSQE
STRYLYGEGDLNFSDDSGISGLRNHMTSLNLDLQSQNIYHHGREGSRMN
GSRTLTHSISDGQLQGGQSNSELFQQEPQTAPAQVPQGENVFGMSSSSG
ASNSAPHLGFHLGSKGTSSLSQQTPRFNPIMVTLAPNIQTGRNTPTSLH
IHGVPPPVLNSPQGNSIYIRPYITTPGGTTRQTQQHSGWVSQFNPMNPQ
QVYQPSQPGPWTTCPASNPLSHTSSQQPNQQGHQTSHVYMPISSPTTSQ
PPTIHSSGSSQSSAHSQYNIQNISTGPRKNQIEIKLEPPQRNNSSKLRS
SGPRTSSTSSSVNSQTLNRNQPTVYIAASPPNTDELMSRSQPKVYISAN
AATGDEQVMRNQPTLFISTNSGASAASRNMSGQVSMGPAFIHHHPPKSR
AIGNNSATSPRVVVTQPNTKYTFKITVSPNKPPAVSPGVVSPTFELTNL
LNHPDHYVETENIQHLTDPTLAHVDRISETRKLSMGSDDAAYTQALLVH
QKARMERLQRELEIQKKKLDKLKSEVNEMENNLTRRRLKRSNSISQIPS
LEEMQQLRSCNRQLQIDIDCLTKEIDLFQARGPHFNPSAIHNFYDNIGF
VGPVPPKPKDQRSIIKTPKTQDTEDDEGAQWNCTACTFLNHPALIRCEQ
CEMPRHF

An exemplary sequence of human TAB3 is shown below:

(UniParc Accession No. UPI0000071648)
SEQ ID NO: 25
MAQSSPQLDIQVLHDLRQRFPEIPEGVVSQCMLQNNNNLEACCRALSQE
SSKYLYMEYHSPDDNRMNRNRLLHINLGIHSPSSYHPGDGAQLNGGRTL
VHSSSDGHIDPQHAAGKQLICLVQEPHSAPAVVAATPNYNPFFMNEQNR
SAATPPSQPPQQPSSMQTGMNPSAMQGPSPPPPPPSYMHIPRYSTNPIT
VTVSQNLPSGQTVPRALQILPQIPSNLYGSPGSIYIRQTSQSSSGRQTP
QSTPWQSSPQGPVPHYSQRPLPVYPHQQNYQPSQYSPKQQQIPQSAYHS
PPPSQCPSPFSSPQHQVQPSQLGHIFMPPSPSTTPPHPYQQGPPSYQKQ
GSHSVAYLPYTASSLSKGSMKKIEITVEPSQRPGTAINRSPSPISNQPS
PRNQHSLYTATTPPSSSPSRGISSQPKPPFSVNPVYITYTQPTGPSCTP
SPSPRVIPNPTTVFKITVGRATTENLLNLVDQEERSAAPEPIQPISVIP
GSGGEKGSHKYQRSSSSGSDDYAYTQALLLHQRARMERLAKQLKLEKEE
LERLKSEVNGMEHDLMQRRLRRVSCTTAIPTPEEMTRLRSMNRQLQINV
DCTLKEVDLLQSRGNFDPKAMNNFYDNIEPGPVVPPKPSKKDSSDPCTI
ERKARRISVTSKVQADIHDTQAAAADEHRTGSTQSPRTQPRDEDYEGAP
WNCDSCTFLNHPALNRCEQCEMPRYT

An exemplary sequence of human MKK7 is shown below:

(UniParc Accession No. UPI000012F494)
SEQ ID NO: 26
MAASSLEQKLSRLEAKLKQENREARRRIDLNLDISPQRPRPTLQLPLAN
DGGSRSPSSESSPQHPTPPARPRHMLGLPSTLFTPRSMESIEIDQKLQE
IMKQTGYLTIGGQRYQAEINDLENLGEMGSGTCGQVWKMRFRKTGHVIA
VKQMRRSGNKEENKRILMDLDVVLKSHDCPYIVQCFGTFITNTDVFIAM
ELMGTCAEKLKKRMQGPIPERILGKMTVAIVKALYYLKEKHGVIHRDVK
PSNILLDERGQIKLCDFGISGRLVDSKAKTRSAGCAAYMAPERIDPPDP
TKPDYDIRADVWSLGISLVELATGQFPYKNCKTDFEVLTKVLQEEPPLL
PGHMGFSGDFQSFVKDCLTKDHRKRPKYNKLLEHSFIKRYETLEVDVAS
WFKDVMAKTESPRTSGVLSQPHLPFFR

An exemplary sequence of human IKKα is shown below:

(UniParc Accession No. UPI000013D6C7)
SEQ ID NO: 27
MERPPGLRPGAGGPWEMRERLGTGGFGNVCLYQHRELDLKIAIKSCRLE
LSTKNRERWCHEIQIMKKLNHANVVKACDVPEELNILIHDVPLLAMEYC
SGGDLRKLLNKPENCCGLKESQILSLLSDIGSGIRYLHENKIIHRDLKP
ENIVLQDVGGKIIHKIIDLGYAKDVDQGSLCTSFVGTLQYLAPELFENK
PYTATVDYWSFGTMVFECIAGYRPFLHHLQPFTWHEKIKKKDPKCIFAC
EEMSGEVRFSSHLPQPNSLCSLVVEPMENWLQLMLNWDPQQRGGPVDLT
LKQPRCFVLMDHILNLKIVHILNMTSAKIISFLLPPDESLHSLQSRIER
ETGINTGSQELLSETGISLDPRKPASQCVLDGVRGCDSYMVYLFDKSKT
VYEGPFASRSLSDCVNYIVQDSKIQLPIIQLRKVWAEAVHYVSGLKEDY
SRLFQGQRAAMLSLLRYNANLIKMKNILISASQQLKAKLEFFHKSIQLD
LERYSEQMTYGISSEKMLKAWKEMEEKAIHYAEVGVIGYLEDQIMSLHA
EIMELQKSPYGRRQGDLMESLEQRAIDLYKQLKHRPSDHSYSDSTEMVK
IIVHTVQSQDRVLKELFGHLSKLLGCKQKIIDLLPKVEVALSNIKEADN
TVMFMQGKRQKEIWHLLKIACTQSSARSLVGSSLEGAVTPQTSAWLPPT
SAEHDHSLSCVVTPQDGETSAQMIEENLNCLGHLSTIIHEANEEQGNSM
MNLDWSWLTE

An exemplary sequence of human IKKβ is shown below:

(UniParc Accession No. UPI0000033729)
SEQ ID NO: 28
MSWSPSLTTQTCGAWEMKERLGTGGFGNVIRWHNQETGEQIAIKQCRQE
LSPRNRERWCLEIQIMRRLTHPNVVAARDVPEGMQNLAPNDLPLLAMEY
CQGGDLRKYLNQFENCCGLREGAILTLLSDIASALRYLHENRIIHRDLK
PENIVLQQGEQRLIHKIIDLGYAKELDQGSLCTSFVGTLQYLAPELLEQ
QKYTVTVDYWSFGTLAFECITGFRPFLPNWQPVQWHSKVRQKSEVDIVV
SEDLNGTVKFSSSLPYPNNLNSVLAERLEKWLQLMLMWHPRQRGTDPTY
GPNGCFKALDDILNLKLVHILNMVTGTIHTYPVTEDESLQSLKARIQQD
TGIPEEDQELLQEAGLALIPDKPATQCISDGKLNEGHTLDMDLVFLFDN
SKITYETQISPRPQPESVSCILQEPKRNLAFFQLRKVWGQVWHSIQTLK
EDCNRLQQGQRAAMMNLLRNNSCLSKMKNSMASMSQQLKAKLDFFKTSI
QIDLEKYSEQTEFGITSDKLLLAWREMEQAVELCGRENEVKLLVERMMA
LQTDIVDLQRSPMGRKQGGTLDDLEEQARELYRRLREKPRDQRTEGDSQ
EMVRLLLQAIQSFEKKVRVIYTQLSKTVVCKQKALELLPKVEEVVSLMN
EDEKTVVRLQEKRQKELWNLLKIACSKVRGPVSGSPDSMNASRLSQPGQ
LMSQPSTASNSLPEPAKKSEELVAEAHNLCTLLENAIQDTVREQDQSFT
ALDWSWLQTEEEEHSCLEQAS

An exemplary sequence of human IKKγ is shown below:

(UniParc Accession No. UPI0000000CC4)
SEQ ID NO: 29
MNRHLWKSQLCEMVQPSGGPAADQDVLGEESPLGKPAMLHLPSEQGAPE
TLQRCLEENQELRDAIRQSNQILRERCEELLHFQASQREEKEFLMCKFQ
EARKLVERLGLEKLDLKRQKEQALREVEHLKRCQQQMAEDKASVKAQVT
SLLGELQESQSRLEAATKECQALEGRARAASEQARQLESEREALQQQHS
VQVDQLRMQGQSVEAALRMERQAASEEKRKLAQLQVAYHQLFQEYDNHI
KSSVVGSERKRGMQLEDLKQQLQQAEEALVAKQEVIDKLKEEAEQHKIV
METVPVLKAQADIYKADFQAERQAREKLAEKKELLQEQLEQLQREYSKL
KASCQESARIEDMRKRHVEVSQAPLPPAPAYLSSPLALPSQRRSPPEEP
PDFCCPKCQYQAPDMDTLQIHVMECIE

Non-limiting examples of dysregulation of an IKKγ gene or an IKKγ protein are described in, for example, Courtois and Gilmore, Oncogene 25.51 (2006): 6831-6843.

An exemplary sequence of human IκBa is shown below:

(UniParc Accession No. UPI000004F0A9)
SEQ ID NO: 30
MFQAAERPQEWAMEGPRDGLKKERLLDDRHDSGLDSMKDEEYEQMVKEL
QEIRLEPQEVPRGSEPWKQQLTEDGDSFLHLAIIHEEKALTMEVIRQVK
GDLAFLNFQNNLQQTPLHLAVITNQPEIAEALLGAGCDPELRDFRGNTP
LHLACEQGCLASVGVLTQSCTTPHLHSILKATNYNGHTCLHLASIHGYL
GIVELLVSLGADVNAQEPCNGRTALHLAVDLQNPDLVSLLLKCGADVNR
VTYQGYSPYQLTWGRPSTRIQQQLGQLTLENLQMLPESEDEESYDTESE
FTEFTEDELPYDDCVFGGQRLTL

An exemplary sequence of human p105, which is processed into p50, is shown below:

(UniParc Accession No. UPI000000D917)
SEQ ID NO: 31
MAEDDPYLGRPEQMFHLDPSLTHTIFNPEVFQPQMALPTDGPYLQILEQ
PKQRGFRFRYVCEGPSHGGLPGASSEKNKKSYPQVKICNYVGPAKVIVQ
LVINGKNIHLHAHSLVGKHCEDGICTVTAGPKDMVVGFANLGILHVTKK
KVFETLEARMTEACIRGYNPGLLVHPDLAYLQAEGGGDRQLGDREKELI
RQAALQQTKEMDLSVVRLMFTAFLPDSTGSFTRRLEPVVSDAIYDSKAP
NASNLKIVRMDRTAGCVTGGEEIYLLCDKVQKDDIQIRFYEEEENGGVW
EGFGDFSPIDVHRQFAIVFKTPKYKDINITKPASVFVQLRRKSDLETSE
PKPFLYYPEIKDKEEVQRKRQKLMPNFSDSFGGGSGAGAGGGGMFGSGG
GGGGTGSTGPGYSFPHYGFPTYGGITFHPGTTKSNAGMKHGTMDTESKK
DPEGCDKSDDKNTVNLFGKVIETTEQDQEPSEATVGNGEVTLTYATGTK
EESAGVQDNLFLEKAMQLAKRHANALFDYAVTGDVKMLLAVQRHLTAVQ
DENGDSVLHLAIIHLHSQLVRDLLEVTSGLISDDIINMRNDLYQTPLHL
AVITKQEDVVEDLLRAGADLSLLDRLGNSVLHLAAKEGHDKVLSILLKH
KKAALLLDHPNGDGLNAIHLAMMSNSLPCLLLLVAAGADVNAQEQKSGR
TALHLAVEHDNISLAGCLLLEGDAHVDSTTYDGTTPLHIAAGRGSTRLA
ALLKAAGADPLVENFEPLYDLDDSWENAGEDEGVVPGTTPLDMATSWQV
FDILNGKPYEPEFTSDDLLAQGDMKQLAEDVKLQLYKLLEIPDPDKNWA
TLAQKLGLGILNNAFRLSPAPSKTLMDNYEVSGGTVRELVEALRQMGYT
EAIEVIQAASSPVKTTSQAHSLPLSPASTRQQIDELRDSDSVCDSGVET
SFRKLSFTESLTSGASLLTLNKMPHDYGQEGPLEGKI

An exemplary sequence of human p65 is shown below:

(UniParc Accession No. UPI000013ED68)
SEQ ID NO: 32
MDELFPLIFPAEPAQASGPYVEIIEQPKQRGMRFRYKCEGRSAGSIPGE
RSTDTTKTHPTIKINGYTGPGTVRISLVTKDPPHRPHPHELVGKDCRDG
FYEAELCPDRCIHSFQNLGIQCVKKRDLEQAISQRIQTNNNPFQVPIEE
QRGDYDLNAVRLCFQVTVRDPSGRPLRLPPVLSHPIFDNRAPNTAELKI
CRVNRNSGSCLGGDEIFLLCDKVQKEDIEVYFTGPGWEARGSFSQADVH
RQVAIVFRTPPYADPSLQAPVRVSMQLRRPSDRELSEPMEFQYLPDTDD
RHRIEEKRKRTYETFKSIMKKSPFSGPTDPRPPPRRIAVPSRSSASVPK
PAPQPYPFTSSLSTINYDEFPTMVFPSGQISQASALAPAPPQVLPQAPA
PAPAPAMVSALAQAPAPVPVLAPGPPQAVAPPAPKPTQAGEGTLSEALL
QLQFDDEDLGALLGNSTDPAVFTDLASVDNSEFQQLLNQGIPVAPHTTE
PMLMEYPEAITRLVTGAQRPPDPAPAPLGAPGLPNGLLSGDEDFSSIAD
MDFSALLSQISS

An exemplary sequence of human c-Rel is shown below:

(UniParc Accession No. UPI000013367B)
SEQ ID NO: 33
MASGAYNPYIEIIEQPRQRGMRFRYKCEGRSAGSIPGEHSTDNNRTYPS
IQIMNYYGKGKVRITLVTKNDPYKPHPHDLVGKDCRDGYYEAEFGQERR
PLFFQNLGIRCVKKKEVKEAIITRIKAGINPFNVPEKQLNDIEDCDLNV
VRLCFQVFLPDEHGNLTTALPPVVSNPIYDNRAPNTAELRICRVNKNCG
SVRGGDEIFLLCDKVQKDDIEVRFVLNDWEAKGIFSQADVHRQVAIVFK
TPPYCKAITEPVTVKMQLRRPSDQEVSESMDFRYLPDEKDTYGNKAKKQ
KTTLLFQKLCQDHVETGFRHVDQDGLELLTSGDPPTLASQSAGITVNFP
ERPRPGLLGSIGEGRYFKKEPNLFSHDAVVREMPTGVSSQAESYYPSPG
PISSGLSHHASMAPLPSSSWSSVAHPTPRSGNINPLSSFSTRTLPSNSQ
GIPPFLRIPVGNDLNASNACIYNNADDIVGMEASSMPSADLYGISDPNM
LSNCSVNMMTTSSDSMGETDNPRLLSMNLENPSCNSVLDPRDLRQLHQM
SSSSMSAGANSNTTVFVSQSDAFEGSDFSCADNSMINESGPSNSTNPNS
HGFVQDSQYSGIGSMQNEQLSDSFPYEFFQV

The term “cancer associated with a component of the JNK pathway downstream of a CBM complex” as used herein refers to cancers associated with or having a dysregulation of a gene, a protein, or the expression or activity or level of any (e.g., one or more) of the same associated with a component of the JNK pathway downstream of a CBM complex. In some embodiments, a cancer associated with a component of the JNK pathway downstream of a CBM complex is selected from the group consisting of a JNK1-associated cancer, a JNK2-associated cancer, a JNK3-associated cancer, a MYD88 transcription factor-associated cancer, an AP-1 transcription factor-associated cancer, and combinations thereof. The cancers “associated” with a particular gene or protein described in this paragraph refer to cancers associated with or having a dysregulation of the particular gene, the particular protein, or the expression or activity or level of any (e.g., one or more) of the same (e.g., any of the types of dysregulation of the particular gene, the particular protein, or the expression or activity or level of any of the same described herein). Non-limiting examples of such cancers are described herein.

An exemplary sequence of human JNK1 is shown below:

(UniParc Accession No. UPI000012F17A)
SEQ ID NO: 34
MSRSKRDNNFYSVEIGDSTFTVLKRYQNLKPIGSGAQGIVCAAYDAILE
RNVAIKKLSRPFQNQTHAKRAYRELVLMKCVNHKNIIGLLNVFTPQKSL
EEFQDVYIVMELMDANLCQVIQMELDHERMSYLLYQMLCGIKHLHSAGI
IHRDLKPSNIVVKSDCTLKILDFGLARTAGTSFMMTPYVVTRYYRAPEV
ILGMGYKENVDLWSVGCIMGEMVCHKILFPGRDYIDQWNKVIEQLGTPC
PEFMKKLQPTVRTYVENRPKYAGYSFEKLFPDVLFPADSEHNKLKASQA
RDLLSKMLVIDASKRISVDEALQHPYINVWYDPSEAEAPPPKIPDKQLD
EREHTIEEWKELIYKEVMDLEERIKNGVIRGQPSPLGAAVINGSQHPSS
SSSVNDVSSMSTDPTLASDIDSSLEAAAGPLGCCR

An exemplary sequence of human JNK2 is shown below:

(UniParc Accession No. UPI000006E3AD)
SEQ ID NO: 35
MSDSKCDSQFYSVQVADSTFTVLKRYQQLKPIGSGAQGIVCAAFDTVLG
INVAVKKLSRPFQNQTHAKRAYRELVLLKCVNHKNIISLLNVFTPQKTL
EEFQDVYLVMELMDANLCQVIHMELDHERMSYLLYQMLCGIKHLHSAGI
IHRDLKPSNIVVKSDCTLKILDFGLARTACTNFMMTPYVVTRYYRAPEV
ILGMGYKENVDIWSVGCIMGELVKGCVIFQGTDHIDQWNKVIEQLGTPS
AEFMKKLQPTVRNYVENRPKYPGIKFEELFPDWIFPSESERDKIKTSQA
RDLLSKMLVIDPDKRISVDEALRHPYITVWYDPAEAEAPPPQIYDAQLE
EREHAIEEWKELIYKEVMDWEERSKNGVVKDQPSDAAVSSNATPSQSSS
INDISSMSTEQTLASDTDSSLDASTGPLEGCR

An exemplary sequence of human JNK3 is shown below:

(UniParc Accession No. UPI0000049042)
SEQ ID NO: 36
MSLHFLYYCSEPTLDVKIAFCQGFDKQVDVSYIAKHYNMSKSKVDNQFY
SVEVGDSTFTVLKRYQNLKPIGSGAQGIVCAAYDAVLDRNVAIKKLSRP
FQNQTHAKRAYRELVLMKCVNHKNIISLLNVFTPQKTLEEFQDVYLVME
LMDANLCQVIQMELDHERMSYLLYQMLCGIKHLHSAGIIHRDLKPSNIV
VKSDCTLKILDFGLARTAGTSFMMTPYVVTRYYRAPEVILGMGYKENVD
IWSVGCIMGEMVRHKILFPGRDYIDQWNKVIEQLGTPCPEFMKKLQPTV
RNYVENRPKYAGLTFPKLFPDSLFPADSEHNKLKASQARDLLSKMLVID
PAKRISVDDALQHPYINVWYDPAEVEAPPPQIYDKQLDEREHTIEEWKE
LIYKEVMNSEEKTKNGVVKGQPSPSGAAVNSSESLPPSSSVNDISSMST
DQTLASDTDSSLEASAGPLGCCR

### Compounds of Formula (I)

Provided herein are compounds of Formula (I), or a pharmaceutically acceptable salt thereof:

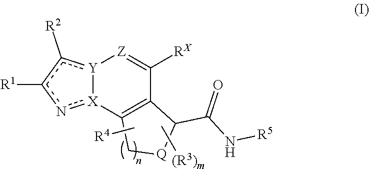

wherein:
each [symbol] is a single or double bond;
Q is —CH₂—,O, or NH;
X is N or C;
Y is N or C;
Z is N or CR⁶;
wherein when one of X and Y is N, the other of X and Y is C;
n is 1, 2, or 3;
R^{X} is hydrogen or halogen;
R¹ is hydrogen, halogen, cyano, hydroxyl, C1-C3 alkoxy, C1-C3 haloalkoxy, C1-C3 haloalkyl, —NR^{A}R^{B}, or C1-C3 alkyl optionally substituted with 1-3 substituents selected from hydroxyl and C1-C3 alkoxy;
R² is hydrogen, halogen, amino, or C1-C3 alkyl;
each R³ is independently deuterium, halogen, C3-C6 cycloalkyl, C1-C3 alkyl, C1-C3 alkoxy, C1-C3 haloalkoxy, or C1-C3 haloalkyl;
m is 0, 1, 2, or 3;
R⁴ is 5-10 membered heteroaryl optionally substituted with one to three substituents each independently selected from R⁷;
R⁵ is phenyl or 5-9 membered heteroaryl, wherein each R⁵ group is optionally substituted with 1-3 substituents independently selected from R⁸;
R⁶ is hydrogen, halogen, cyano, hydroxyl, C1-C3 alkoxy, C1-C3 haloalkoxy, C1-C3 haloalkyl, —NR^{C}R^{D}, and C1-C3 alkyl;
each R⁷ is independently selected from the group consisting of halogen, cyano, hydroxyl, C1-C3 alkoxy, C1-C3 haloalkoxy, C1-C3 haloalkyl, oxetanyl, —NR^{C}R^{D}, C1-C3 alkyl optionally substituted with one cyclopropyl; and C3-C7 cycloalkyl optionally substituted with one C1-C3 alkyl;
each R⁸ is independently selected from halogen; cyano; amino; —N═(S═O)(C1-C3 alkyl)₂; —S(═O)ₚ(C1-C3 alkyl); 1-imino-1-lambda⁶-thietanyl 1-oxide, —(C═O)NR^{E}R^{F}; C1-C3 alkoxy; C1-C3 haloalkyl optionally substituted with hydroxyl; C1-C3 haloalkoxy; 5-6 membered heteroaryl optionally substituted with halogen, cyano, hydroxyl, C1-C3 alkoxy, C1-C3 haloalkoxy, amino, C1-C3 haloalkyl, 4-6 membered heterocyclyl, or C1-C3 alkyl optionally substituted with hydroxyl or —NR^{E}R^{F}; C1-C4 alkyl optionally substituted with hydroxyl, —NR^{E}R^{F}, or C1-C3 alkoxy; 3-8 membered heterocyclyl; and C3-C6 cycloalkoxy;
p is 1 or 2; and
R^{A}, R^{B}, R^{C}, R^{D}, R^{E}, and R^{F}, are independently hydrogen, C1-C3 alkyl, C3-C6 cycloalkyl, or R^{A} and R^{B}, or R^{C} and R^{D}, or R^{E} and R^{F}, together with the nitrogen atom to which they are attached come together to form a 4-6 membered heterocyclyl optionally substituted with 1-2 halogens.

In some embodiments, the compound of Formula (I), or a pharmaceutically acceptable salt thereof, has the structure:

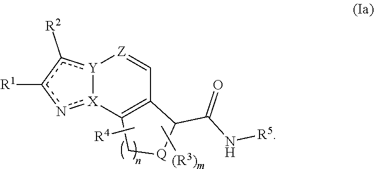

In some embodiments, Q is —CH₂—. In some embodiments, Q is O. In some embodiments, Q is NH.

In some embodiments, the five-membered nitrogen-containing ring, formed in part by X and Y, is a heteroaromatic ring.

In some embodiments, X is C and Y is C.

In some embodiments, X is N and Y is C.

In some embodiments, X is C and Y is N.

In some embodiments, Z is N. In some embodiments, Z is CR⁶. In some embodiments, Z is CR⁶ wherein R⁶ is hydrogen.

In some embodiments, X is C; Y is C; and Z is CR⁶. In some embodiments, X is N; Y is C; and Z is CR⁶. In some embodiments, X is C; Y is N; and Z is CR⁶. In some embodiments, X is C; Y is N; and Z is CR⁶ wherein R⁶ is hydrogen. In some embodiments, X is C; Y is C; and Z is N. In some embodiments, X is N; Y is C; and Z is N. In some embodiments, X is C; Y is N; and Z is N.

In some embodiments, R⁶ is hydrogen.

In some embodiments, R⁶ is halogen. For example, R⁶ is fluoro. For example, R⁶ is chloro.

In some embodiments, R⁶ is cyano. In some embodiments, R⁶ is hydroxyl.

In some embodiments, R⁶ is C1-C3 alkoxy. In some embodiments, R⁶ is methoxy or ethoxy.

In some embodiments, R⁶ is C1-C3 haloalkoxy. In some embodiments, R⁶ is trifluoromethoxy, difluoromethoxy, or fluoromethoxy.

In some embodiments, R⁶ is C1-C3 haloalkyl. In some embodiments, R⁶ is trifluoromethyl or 2,2,2-trifluoroethyl.

In some embodiments, R¹ is halogen, cyano, hydroxyl, C1-C3 alkoxy, C1-C3 haloalkyl, —NR^{A}R^{B}, or C1-C3 alkyl optionally substituted with 1-3 substituents selected from hydroxyl and C1-C3 alkoxy.

In some embodiments, R¹ is halogen or cyano. In some embodiments, R¹ is chloro or cyano. In some embodiments, R¹ is hydrogen. In some embodiments, R¹ is halogen. In some embodiments, R¹ is fluoro or chloro. In some embodiments, R¹ is fluoro. In some embodiments, R¹ is chloro. In some embodiments, R¹ is cyano. In some embodiments, R¹ is hydroxyl.

In some embodiments, R¹ is C1-C3 alkoxy. In some embodiments, R¹ is methoxy or ethoxy.

In some embodiments, R¹ is C1-C3 haloalkoxy. In some embodiments, R¹ is trifluoromethoxy, difluoromethoxy, or fluoromethoxy.

In some embodiments, R¹ is C1-C3 haloalkyl. In some embodiments, R¹ is difluoromethyl, trifluoromethyl or 2,2,2-trifluoroethyl.

In some embodiments, R¹ is C1-C3 alkyl optionally substituted with 1-3 substituents selected from hydroxyl and C1-C3 alkoxy.

In some embodiments, R² is hydrogen. In some embodiments, R² is halogen. In some embodiments, R² is fluoro. In some embodiments, R² is chloro. In some embodiments, R² is amino. In some embodiments, R² is C1-C3 alkyl, such as methyl.

In some embodiments, n is 1, 2, or 3. In some embodiments, n is 1 or 2. In some embodiments, n is 2 or 3. In some embodiments, n is 1 or 3. In some embodiments, n is 1. In some embodiments, n is 2. In some embodiments, n is 3.

In some embodiments, m is 0, 1, 2, or 3. In some embodiments, m is 0, 1, or 2. In some embodiments, m is 1, 2, or 3. In some embodiments, m is 0, 2, or 3. In some embodiments, m is 0, 1, or 3. In some embodiments, m is 0 or 1. In some embodiments, m is 0 or 2. In some embodiments, m is 0 or 3. In some embodiments, m is 1 or 2. In some embodiments, m is 1 or 3.

In some embodiments, m is 2 or 3. In some embodiments, m is 0. In some embodiments, m is 1.

In some embodiments, m is 2. In some embodiments, m is 3.

In some embodiments, each R³ is independently deuterium, halogen, C3-C6 cycloalkyl, C1-C3 alkyl, C1-C3 haloalkyl, C1-C3 alkoxy, or C1-C3 haloalkoxy.

In some embodiments, each R³ is independently halogen. In some embodiments, R³ is fluoro. In some embodiments, R³ is chloro. In some embodiments, each R³ is independently hydroxyl.

In some embodiments, each R³ is independently C3-C6 cycloalkyl, C1-C3 alkyl, C1-C3 alkoxy, C1-C3 haloalkoxy, or C1-C3 haloalkyl.

In some embodiments, each R³ is independently C1-C3 alkyl. For example, R³ is methyl or ethyl. In some embodiments, each R³ is independently C1-C3 alkoxy. For example, R³ is methoxy or ethoxy. In some embodiments, each R³ is independently C1-C3 haloalkoxy. For example, R³ is trifluoromethoxy, difluoromethoxy, or fluoromethoxy. In some embodiments, each R³ is independently C1-C3 haloalkyl. For example, each R³ is trifluoromethyl or 2,2,2-trifluoroethyl.

In some embodiments, m is 1 and R³ is methyl or trifluoromethyl.

In some embodiments, R⁴ is a 5 or 6 membered heteroaryl optionally substituted with one to three substituents each independently selected from R⁷.

In some embodiments, R⁴ is a 6 membered heteroaryl optionally substituted with one to three substituents each independently selected from R⁷.

In some embodiments, R⁴ is a 5 membered heteroaryl optionally substituted with one to three substituents each independently selected from R⁷.

In some embodiments, R⁴ is 4H-pyrazolyl, 3H-pyrazolyl, or thiazoyl optionally substituted with one to three substituents each independently selected from R⁷.

In some embodiments R⁴ is unsubstituted. For example, R⁴ is unsubstituted 4H-pyrazolyl, 3H-pyrazolyl, or thiazolyl.

In some embodiments R⁴ is a 5 or 6 membered heteroaryl substituted with one or three substituents each independently selected from R⁷. In other embodiments, R⁴ is a 5 or 6 membered heteroaryl substituted with one substituent selected from R⁷.

In some embodiments, each R⁷ is independently selected from the group consisting of C1-C3 haloalkyl, C1-C3 alkyl, and cycloalkyl. In some embodiments R⁷ is methyl, ethyl, cyclopropyl, or difluoromethyl.

In some embodiments, each R⁷ is independently selected from the group consisting of C1-C3 alkyl optionally substituted with one cyclopropyl.

In some embodiments, each R⁷ is independently selected from the group consisting of C3-C7 cycloalkyl optionally substituted with one C1-C3 alkyl. In some embodiments the C3-C7 alkyl is cyclopropyl.

In some embodiments, R⁴ is:

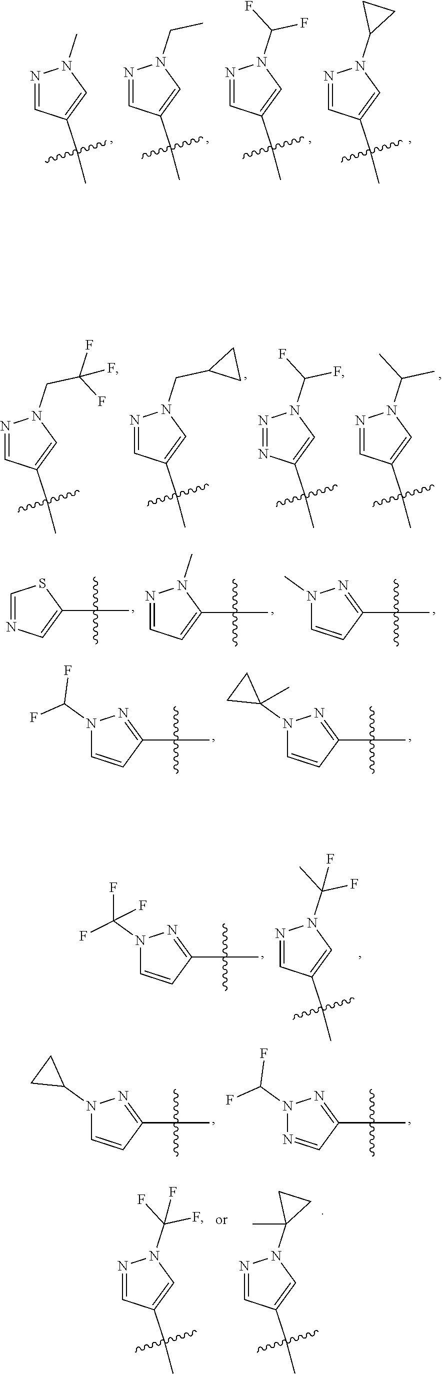

In some embodiments, R⁴ is:

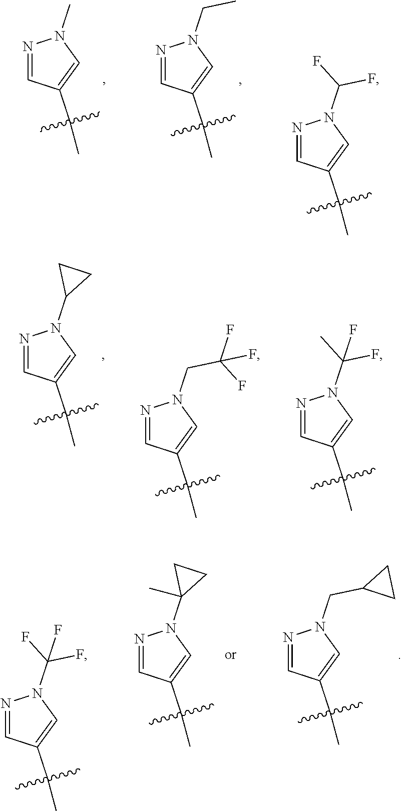

In some embodiments, R⁴ is:

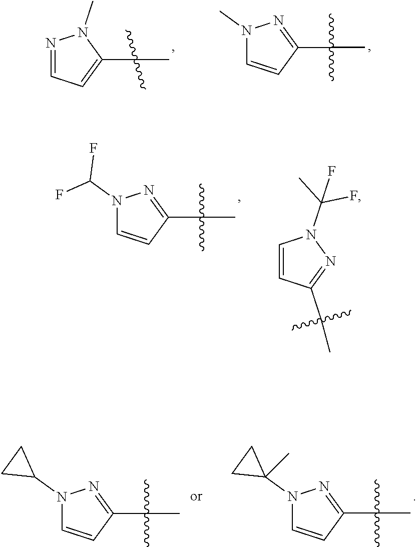

In some embodiments, R⁴ is:

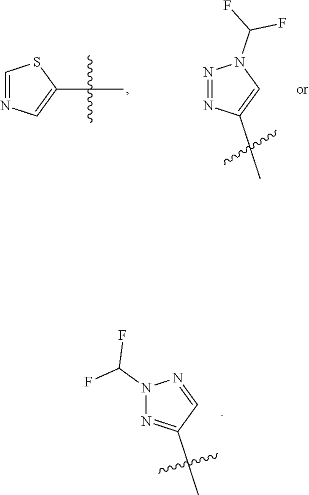 [SMILES: *c1cncs1 ; *c1cn(C(F)F)nn1 ; *c1cnn(C(F)F)n1]

In some embodiments, R⁴ is:

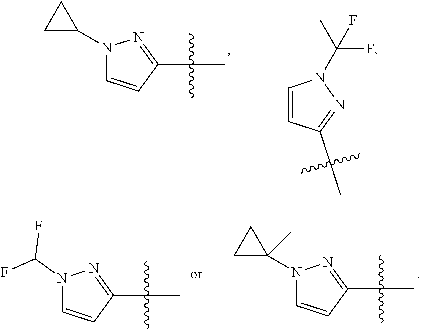

In some embodiments, the compound of Formula (I), or a pharmaceutically acceptable salt thereof, has the structure:

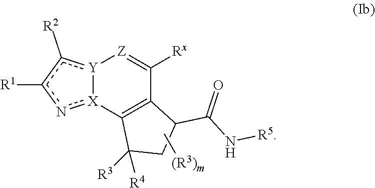

In some embodiments, the compound of Formula (I), or a pharmaceutically acceptable salt thereof, has the structure:

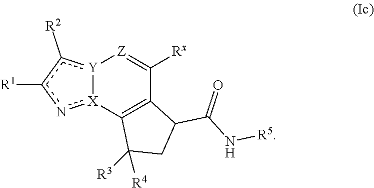

In some embodiments, R⁵ is phenyl or 5-6 membered heteroaryl; wherein each R⁵ group is optionally substituted with 1-2 independently selected R⁸. In some embodiments, R⁵ is phenyl or 5-6 membered heteroaryl; wherein each R⁵ group is optionally substituted with 2-3 independently selected R⁸. In some embodiments, R⁵ is phenyl or 5-6 membered heteroaryl; wherein each R⁵ group is optionally substituted with 1 to 3 independently selected R⁸. In some embodiments, R⁵ is phenyl or 5-6 membered heteroaryl; wherein each R⁵ group is optionally substituted with 1 R⁸.

In some embodiments, R⁵ is phenyl or 5-6 membered heteroaryl; wherein each R⁸ group is optionally substituted with 2 independently selected R⁸. In some embodiments, R⁵ is phenyl or 5-6 membered heteroaryl; wherein each R⁵ group is optionally substituted with 3 independently selected R⁸.

In some embodiments, R⁵ is phenyl or 5 membered heteroaryl; wherein each R⁵ group is optionally substituted with 1-3 substituents independently selected from R⁸. In some embodiments, R⁵ is phenyl or 6 membered heteroaryl; wherein each R⁵ group is optionally substituted with 1-3 substituents independently selected from R⁸.

In some embodiments, R⁵ is phenyl optionally substituted with 1-3 independently selected R⁸. In certain embodiments, R⁵ is phenyl optionally substituted with 1 R⁸. In certain embodiments, R⁵ is phenyl optionally substituted with 2 independently selected R⁸. In certain embodiments, R⁵ is phenyl optionally substituted with 3 independently selected R⁸.

In some embodiments, R⁵ is unsubstituted phenyl.

In some embodiments, R⁵ is 5-6 membered heteroaryl optionally substituted with 1-3 (e.g., 2) substituents independently selected from R⁸. In some embodiments, R⁵ is 6 membered heteroaryl optionally substituted with 1-3 (e.g., 2) substituents independently selected from R⁸.

In some embodiments, R⁵ is unsubstituted 5-6 membered heteroaryl.

In some embodiments, R⁵ is 5-6 membered heteroaryl substituted with 1-3 substituents independently selected from R⁸.

In some embodiments, R⁵ is unsubstituted 5-6 membered heteroaryl. In some embodiments, at least one of R⁸ is halogen. In some embodiments, at least one of R⁸ is fluoro. In some embodiments, at least one of R⁸ is chloro. In some embodiments, one of R⁸ is halogen. In some embodiments, one of R⁵ is fluoro. In some embodiments, one of R⁵ is chloro. In some embodiments, two of R⁸ is halogen. In some embodiments, two of R⁸ is fluoro. In some embodiments, two of R⁸ is chloro. In some embodiments, three of R⁸ is halogen. In some embodiments, three of R⁸ is fluoro. In some embodiments, three of R⁸ is chloro. In some embodiments, at least one of R⁸ is cyano. In some embodiments, at least one of R⁸ is amino.

In some embodiments, at least one of R⁸ is C1-C3 alkoxy. In some embodiments, at least one of R⁸ is methoxy or ethoxy.

In some embodiments, at least one of R⁸ is C1-C3 haloalkyl optionally substituted with hydroxyl. In some embodiments, at least one of R⁸ is unsubstituted C1-C3 haloalkyl. In some embodiments, at least one of R⁸ is C1-C3 haloalkyl substituted with hydroxyl. In some embodiments, at least one of R⁸ is trifluoromethyl, 2,2-difluoroethyl, or 2,2,2-trifluoroethyl. In some embodiments, at least one of R⁸ is 1-hydroxy-2,2-difluoroethyl.

In some embodiments, at least one of R⁵ is C1-C3 haloalkoxy. In some embodiments, at least one of R⁸ is trifluoromethoxy.

In some embodiments, at least one of R⁸ is 5-6 membered heteroaryl optionally substituted with halogen, cyano, hydroxyl, C1-C3 alkoxy, C1-C3 haloalkoxy, amino, C1-C3 haloalkyl, 4-6 membered heterocyclyl, or C1-C3 alkyl optionally substituted with hydroxyl or —NR^{E}R^{F}. In some embodiments, at least one of R⁸ is 5-6 membered heteroaryl optionally substituted with halogen, cyano, hydroxyl, C1-C3 alkoxy, C1-C3 haloalkoxy, C1-C3 alkyl optionally substituted with hydroxyl or —NR^{E}R^{F}, amino, or C1-C3 haloalkyl. In some embodiments, R⁸ is 5-6 membered heteroaryl optionally substituted with C1-C3 alkyl optionally substituted with hydroxyl or —NR^{E}R^{F}.

In some embodiments, at least one of R⁸ is 5-6 membered heteroaryl optionally substituted with halogen, C1-C3 haloalkyl, or C1-C3 alkyl optionally substituted with hydroxyl or —NR^{E}R^{F}. In some embodiments, R⁸ is 5-6 membered heteroaryl substituted with C1-C3 alkyl substituted with hydroxyl or —NR^{E}R^{F}. In some embodiments, R⁸ is 5-6 membered heteroaryl substituted with hydroxymethyl, aminomethyl, hydroxyethyl, aminoethyl, propan-2-ol, or propan-2-amine. In some embodiments, at least one of R⁸ is 5-6 membered heteroaryl optionally substituted with a 4-6 membered heterocyclyl.

In some embodiments, R⁵ is pyridyl, pyrimidinyl, pyrazinyl, pyrrolyl, or imidazolyl; each of which is substituted with two R⁸: one R⁸ is triazolyl, imidazolyl, oxazolyl, pyrazolyl, or pyrrolidinyl; and the other R⁵ is methyl, methoxy, trifluoromethyl, trifluoromethoxy, chloro, or cyano. In some embodiments, R⁵ is pyridyl, pyrimidinyl, or pyrazinyl; each of which is substituted with two R⁵: one R⁸ is triazolyl, imidazolyl, oxazolyl, pyrazolyl, or pyrrolidinyl; and the other R⁸ is methyl, methoxy, trifluoromethyl, trifluoromethoxy, chloro, or cyano. In some embodiments, R⁵ is pyridyl substituted with two R⁸, one R⁸ is triazolyl, imidazolyl, or oxazolyl; and the other R⁵ is methyl, methoxy, trifluoromethyl, trifluoromethoxy, chloro, or cyano.

In some embodiments, R⁵ is 3-pyridyl or 4-pyridyl substituted with 1-3 independently selected R⁸.

In some embodiments, R⁵ is substituted with two R⁸ wherein one R⁸ is selected from the group consisting of: cyano, fluoro, chloro, methyl, ethyl, methoxy, trifluoromethyl, and the other R⁸ is selected from the group consisting of: 1,2,3-triazol-2-yl, 4-methyl-1,2,3-triazol-2-yl, 4-methyl-1,2,3-triazol-1-yl, 4-amino-1,2,3-triazol-2-yl, 5-cyano-1,2,3-triazol-1-yl, 1,2,3-triazol-1-yl, 3-methyl-1,2,4-triazol-1-yl, 5-methyl-1,2,4-triazol-1-yl, 5-amino-1,2,4-triazol-1-yl, 1-methyl-5-amino-1,2,4-triazol-3-yl, 1,2,4-triazol-4-on-2-yl, tetrazol-5-yl, 2-methyl-tetrazol-5-yl, 1-methyl-tetrazol-5-yl, imidazol-1-yl, 1-methyl-imidazol-3-yl, 1-methyl-5-amino-imidazol-3-yl, 3-methylimidazol-2-on-1-yl, 1-methyl-pyrazol-3-yl, 1-methyl-pyrazol-4-yl, 1-methyl-pyrazol-5-yl, pyrrol-1-yl, thiazol-2-yl, isothiazolidin-2-yl-1,1-dioxide, pyrrolidin-2-on-1-yl, oxazol-2-yl, oxadiazol-2-yl, 2-amino-pyrimidin-4-yl, —(C═O)4-methylpiperazin-1-yl, 2-oxoazetidin-1-yl, azetidin-1-yl, —(C═O)N(CH₃)₂, —(C═O)NHCH₃, —(C═O)NHCH₂CH₃, —(C═O)NHCyclopropyl, —(C═O)(3,3-difluoroazetidin-1-y), 2-hydroxypropan-2-yl, 1-hydroxyethy, dimethyl(oxo)-λ⁶-sulfaneylidene, methoxy, ethoxy, difluoromethoxy, methyl, cyano.

In some embodiments, R⁵ is:

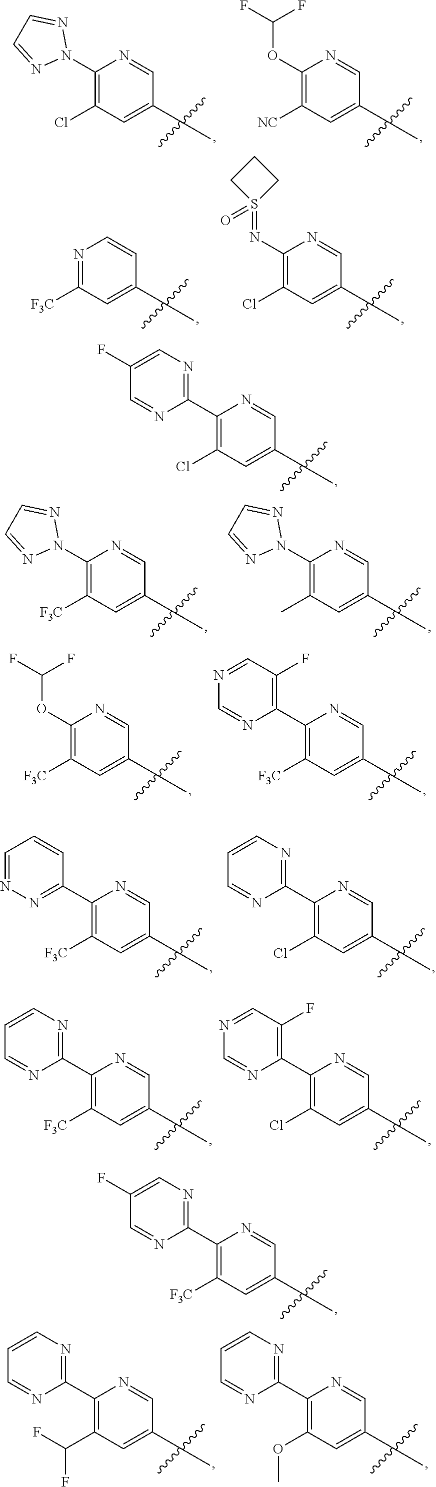

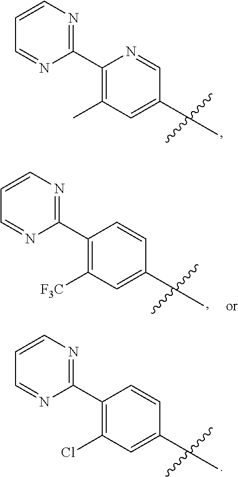 [SMILES: *c1cnc(-c2ncccn2)c(C)c1 ; *c1ccc(-c2ncccn2)c(C(F)(F)F)c1 ; *c1ccc(-c2ncccn2)c(Cl)c1]

In some embodiments, R⁵ is:

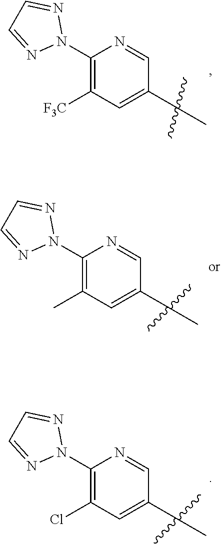 [SMILES: *c1cnc(-n2nccn2)c(C(F)(F)F)c1 ; *c1cnc(-n2nccn2)c(C)c1 ; *c1cnc(-n2nccn2)c(Cl)c1]

In some embodiments, R⁵ is:

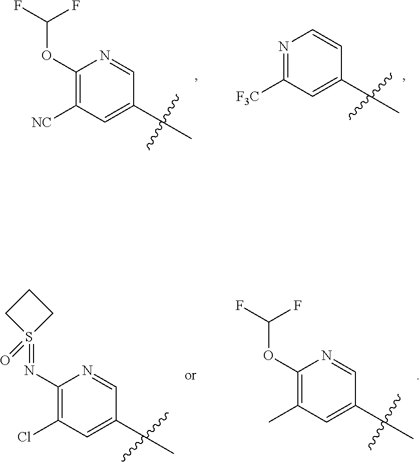 [SMILES: *c1cnc(OC(F)F)c(C#N)c1 ; *c1ccnc(C(F)(F)F)c1 ; *c1cnc(N=S2(=O)CCC2)c(Cl)c1 ; *c1cnc(OC(F)F)c(C)c1]

In some embodiments, R⁵ is:

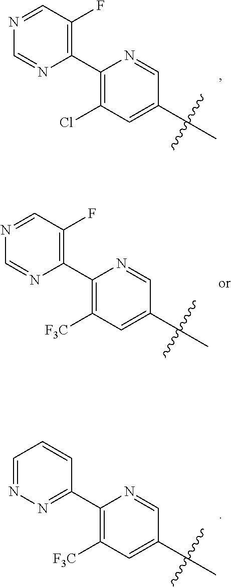 [SMILES: *c1cnc(-c2ncncc2F)c(Cl)c1 ; *c1cnc(-c2ncncc2F)c(C(F)(F)F)c1 ; *c1cnc(-c2cccnn2)c(C(F)(F)F)c1]

In some embodiments, R⁵ is:

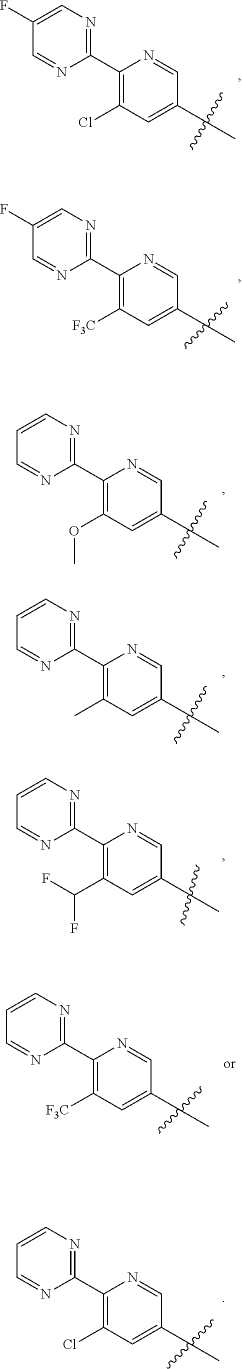

In some embodiments, R⁵ is:

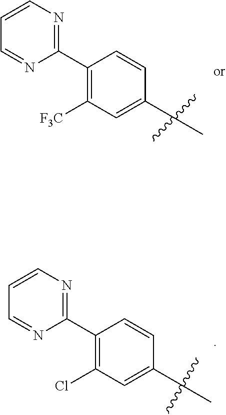 [SMILES: *c1ccc(-c2ncccn2)c(C(F)(F)F)c1 ; *c1ccc(-c2ncccn2)c(Cl)c1]

In some embodiments, R^{X} is hydrogen. In some embodiments, R^{X} is halogen. In some embodiments, R^{X} is fluoro. In some embodiments, R^{X} is chloro.

In some embodiments, R^{A}, R^{B}, R^{C}, R^{D}, R^{E}, and R^{F}, are independently hydrogen or C1-C3 alkyl.

In some embodiments, the compound of Formula (I), or a pharmaceutically acceptable salt thereof, has the structure:

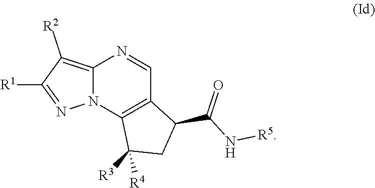

In some embodiments, the compound of Formula (I), or a pharmaceutically acceptable salt thereof, has the structure:

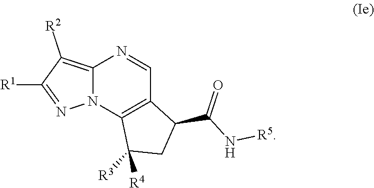

In some embodiments, the compound of Formula (I), or a pharmaceutically acceptable salt thereof, has the structure:

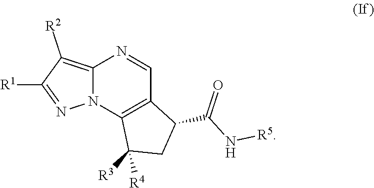

In some embodiments, the compound of Formula (I), or a pharmaceutically acceptable salt thereof, has the structure:

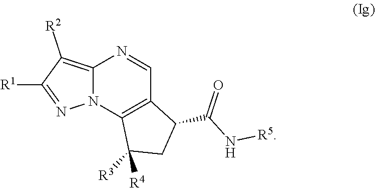

TABLE 1
Representative examples include the following:

| Example | | |
|---|---|---|
| Number | Structure | |
| 1 (first eluting isomer)* 2 (second eluting isomer)* | 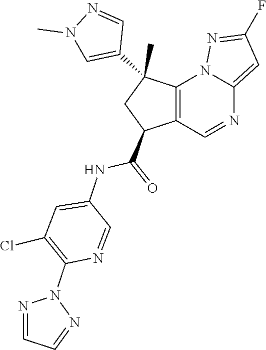 [SMILES: Cn1cc([C@@]2(C)C[C@H](C(=O)Nc3cnc(-n4nccn4)c(Cl)c3)c3cnc4cc(F)nn4c32)cn1] | 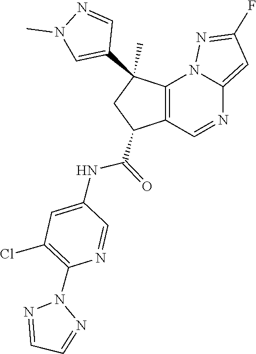 [SMILES: Cn1cc([C@]2(C)C[C@@H](C(=O)Nc3cnc(-n4nccn4)c(Cl)c3)c3cnc4cc(F)nn4c32)cn1] |
| 3 | 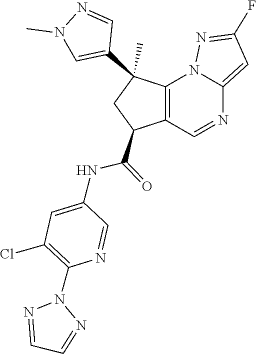 [SMILES: Cn1cc([C@]2(C)C[C@H](C(=O)Nc3cnc(-n4nccn4)c(Cl)c3)c3cnc4cc(F)nn4c32)cn1] | |
| 4 (first eluting isomer)* 5 (second eluting isomer)* | 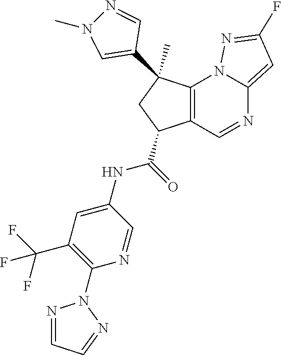 [SMILES: Cn1cc([C@]2(C)C[C@@H](C(=O)Nc3cnc(-n4nccn4)c(C(F)(F)F)c3)c3cnc4cc(F)nn4c32)cn1] | 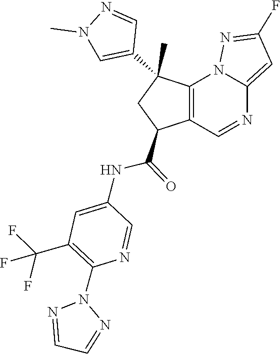 [SMILES: Cn1cc([C@@]2(C)C[C@H](C(=O)Nc3cnc(-n4nccn4)c(C(F)(F)F)c3)c3cnc4cc(F)nn4c32)cn1] |
| 6 (first eluting isomer)* 7 (second eluting isomer)* | 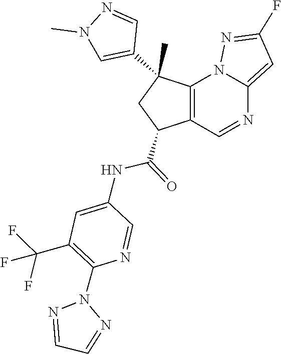 [SMILES: Cn1cc([C@@]2(C)C[C@@H](C(=O)Nc3cnc(-n4nccn4)c(C(F)(F)F)c3)c3cnc4cc(F)nn4c32)cn1] | 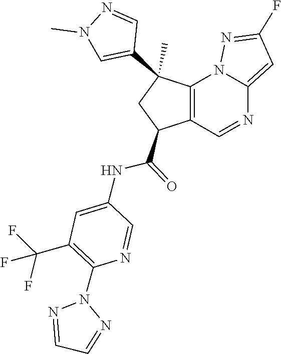 [SMILES: Cn1cc([C@]2(C)C[C@H](C(=O)Nc3cnc(-n4nccn4)c(C(F)(F)F)c3)c3cnc4cc(F)nn4c32)cn1] |
| 8 (second eluting isomer)* 9 first eluting isomer)* | 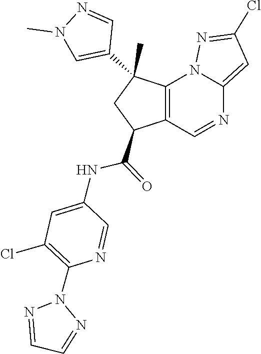 [SMILES: Cn1cc([C@@]2(C)C[C@H](C(=O)Nc3cnc(-n4nccn4)c(Cl)c3)c3cnc4cc(Cl)nn4c32)cn1] | 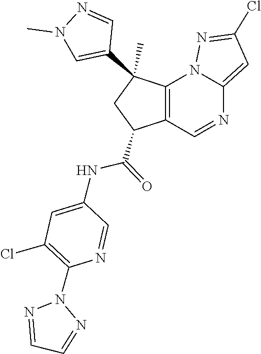 [SMILES: Cn1cc([C@]2(C)C[C@@H](C(=O)Nc3cnc(-n4nccn4)c(Cl)c3)c3cnc4cc(Cl)nn4c32)cn1] |
| 10 (first eluting isomer)* 11 (second eluting isomer)* | 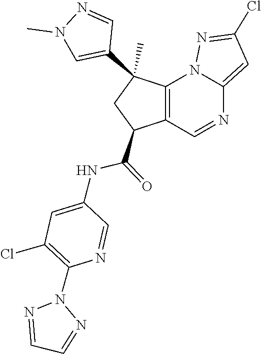 [SMILES: Cn1cc([C@]2(C)C[C@H](C(=O)Nc3cnc(-n4nccn4)c(Cl)c3)c3cnc4cc(Cl)nn4c32)cn1] | 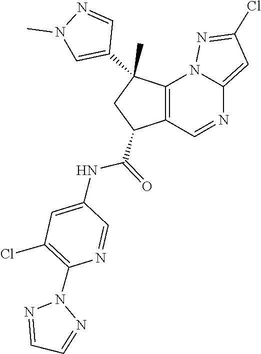 [SMILES: Cn1cc([C@@]2(C)C[C@@H](C(=O)Nc3cnc(-n4nccn4)c(Cl)c3)c3cnc4cc(Cl)nn4c32)cn1] |
| 12 (second eluting isomer)* 13 (first eluting isomer)* | 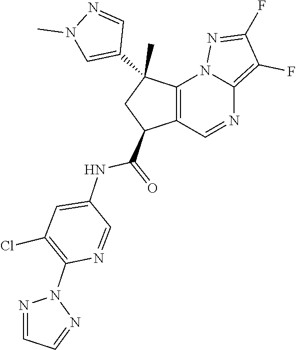 [SMILES: Cn1cc([C@@]2(C)C[C@H](C(=O)Nc3cnc(-n4nccn4)c(Cl)c3)c3cnc4c(F)c(F)nn4c32)cn1] | 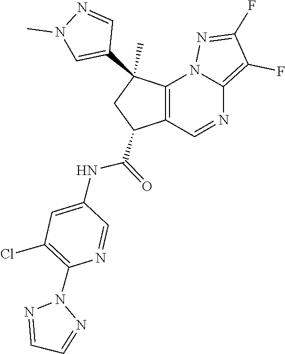 [SMILES: Cn1cc([C@]2(C)C[C@@H](C(=O)Nc3cnc(-n4nccn4)c(Cl)c3)c3cnc4c(F)c(F)nn4c32)cn1] |
| 14 (second eluting isomer)* 15 (first eluting isomer)* | 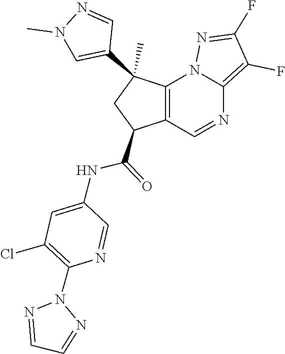 [SMILES: Cn1cc([C@]2(C)C[C@H](C(=O)Nc3cnc(-n4nccn4)c(Cl)c3)c3cnc4c(F)c(F)nn4c32)cn1] | 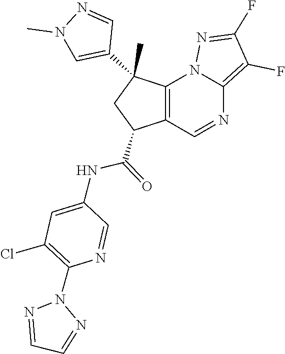 [SMILES: Cn1cc([C@@]2(C)C[C@@H](C(=O)Nc3cnc(-n4nccn4)c(Cl)c3)c3cnc4c(F)c(F)nn4c32)cn1] |
| 16 (second eluting isomer)* 17 (first eluting isomer)* | 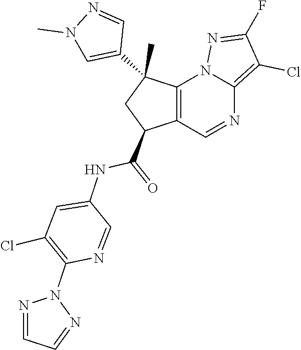 [SMILES: Cn1cc([C@@]2(C)C[C@H](C(=O)Nc3cnc(-n4nccn4)c(Cl)c3)c3cnc4c(Cl)c(F)nn4c32)cn1] | 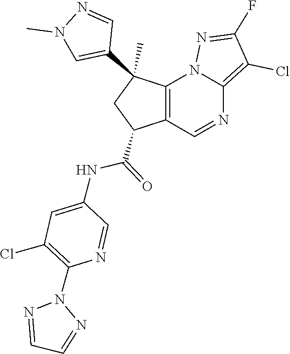 [SMILES: Cn1cc([C@]2(C)C[C@@H](C(=O)Nc3cnc(-n4nccn4)c(Cl)c3)c3cnc4c(Cl)c(F)nn4c32)cn1] |
| 18 (first eluting isomer)* 19 (second eluting isomer)* | 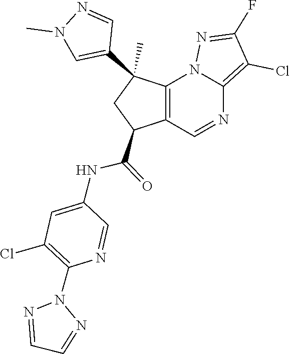 [SMILES: Cn1cc([C@]2(C)C[C@H](C(=O)Nc3cnc(-n4nccn4)c(Cl)c3)c3cnc4c(Cl)c(F)nn4c32)cn1] | 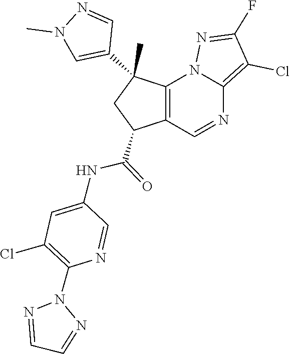 [SMILES: Cn1cc([C@@]2(C)C[C@@H](C(=O)Nc3cnc(-n4nccn4)c(Cl)c3)c3cnc4c(Cl)c(F)nn4c32)cn1] |
| 20 (second eluting isomer)* 21 (first eluting isomer)* | 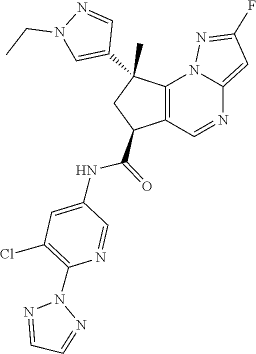 [SMILES: CCn1cc([C@@]2(C)C[C@H](C(=O)Nc3cnc(-n4nccn4)c(Cl)c3)c3cnc4cc(F)nn4c32)cn1] | 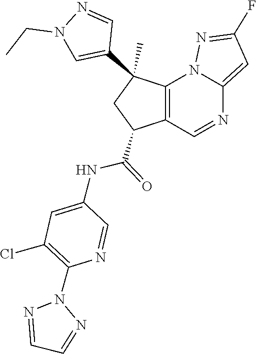 [SMILES: CCn1cc([C@]2(C)C[C@@H](C(=O)Nc3cnc(-n4nccn4)c(Cl)c3)c3cnc4cc(F)nn4c32)cn1] |
| 22 (second eluting isomer)* 23 (first eluting isomer)* | 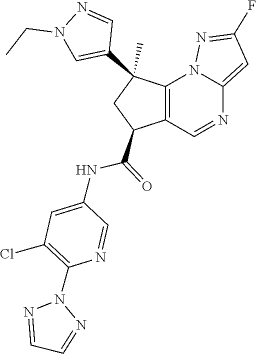 [SMILES: CCn1cc([C@]2(C)C[C@H](C(=O)Nc3cnc(-n4nccn4)c(Cl)c3)c3cnc4cc(F)nn4c32)cn1] | 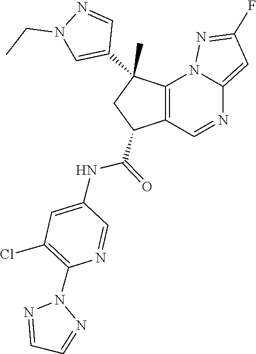 [SMILES: CCn1cc([C@@]2(C)C[C@@H](C(=O)Nc3cnc(-n4nccn4)c(Cl)c3)c3cnc4cc(F)nn4c32)cn1] |
| 24 (second eluting isomer)* 25 (first eluting isomer)* | 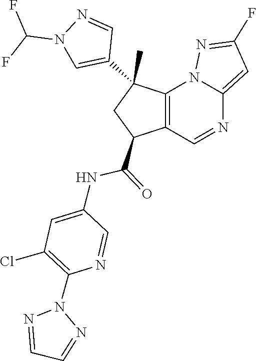 [SMILES: C[C@]1(c2cnn(C(F)F)c2)C[C@H](C(=O)Nc2cnc(-n3nccn3)c(Cl)c2)c2cnc3cc(F)nn3c21] | 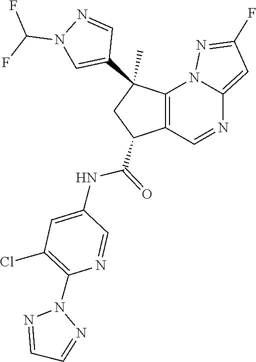 [SMILES: C[C@@]1(c2cnn(C(F)F)c2)C[C@@H](C(=O)Nc2cnc(-n3nccn3)c(Cl)c2)c2cnc3cc(F)nn3c21] |
| 26 (second eluting isomer)* 27 (first eluting isomer)* | 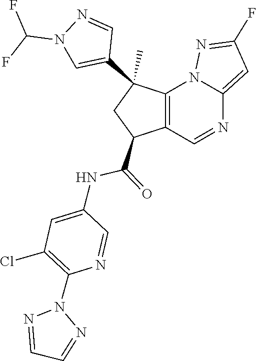 [SMILES: C[C@@]1(c2cnn(C(F)F)c2)C[C@H](C(=O)Nc2cnc(-n3nccn3)c(Cl)c2)c2cnc3cc(F)nn3c21] | 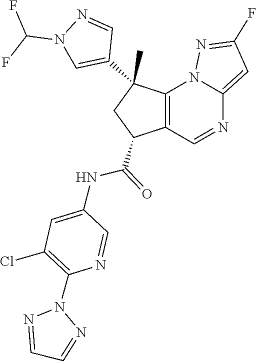 [SMILES: C[C@]1(c2cnn(C(F)F)c2)C[C@@H](C(=O)Nc2cnc(-n3nccn3)c(Cl)c2)c2cnc3cc(F)nn3c21] |
| 28 (second eluting isomer)* 29 (first eluting isomer)* | 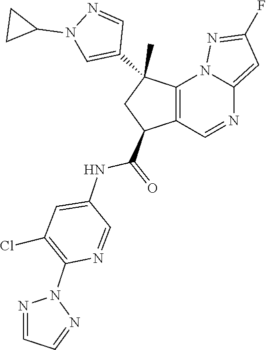 [SMILES: C[C@]1(c2cnn(C3CC3)c2)C[C@H](C(=O)Nc2cnc(-n3nccn3)c(Cl)c2)c2cnc3cc(F)nn3c21] | 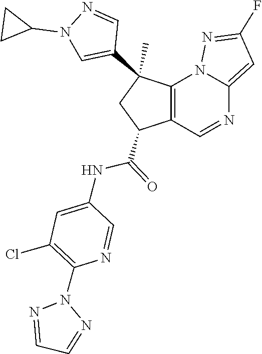 [SMILES: C[C@@]1(c2cnn(C3CC3)c2)C[C@@H](C(=O)Nc2cnc(-n3nccn3)c(Cl)c2)c2cnc3cc(F)nn3c21] |
| 30 (second eluting isomer)* 31 (first eluting isomer)* | 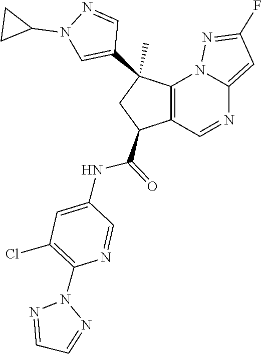 [SMILES: C[C@@]1(c2cnn(C3CC3)c2)C[C@H](C(=O)Nc2cnc(-n3nccn3)c(Cl)c2)c2cnc3cc(F)nn3c21] | 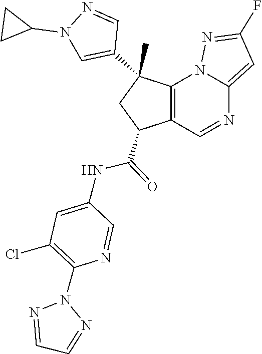 [SMILES: C[C@]1(c2cnn(C3CC3)c2)C[C@@H](C(=O)Nc2cnc(-n3nccn3)c(Cl)c2)c2cnc3cc(F)nn3c21] |
| 32 (second eluting isomer)* 33 (first eluting isomer)* | 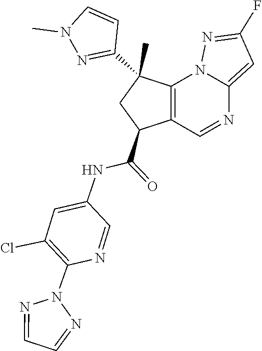 [SMILES: Cn1ccc([C@@]2(C)C[C@H](C(=O)Nc3cnc(-n4nccn4)c(Cl)c3)c3cnc4cc(F)nn4c32)n1] | 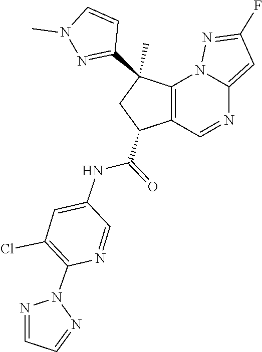 [SMILES: Cn1ccc([C@]2(C)C[C@@H](C(=O)Nc3cnc(-n4nccn4)c(Cl)c3)c3cnc4cc(F)nn4c32)n1] |
| 34 (first eluting isomer)* 35 (second eluting isomer)* | 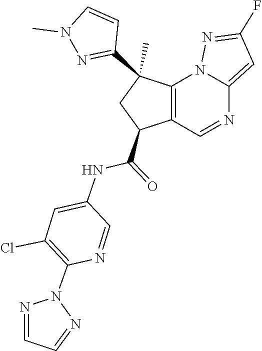 [SMILES: Cn1ccc([C@]2(C)C[C@H](C(=O)Nc3cnc(-n4nccn4)c(Cl)c3)c3cnc4cc(F)nn4c32)n1] | 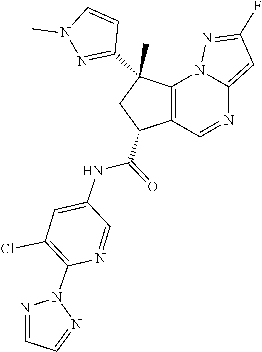 [SMILES: Cn1ccc([C@@]2(C)C[C@@H](C(=O)Nc3cnc(-n4nccn4)c(Cl)c3)c3cnc4cc(F)nn4c32)n1] |
| 36 (second eluting isomer)* 37 (first eluting isomer)* | 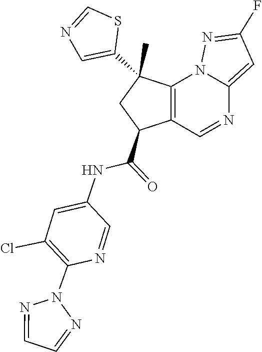 [SMILES: C[C@]1(c2cncs2)C[C@H](C(=O)Nc2cnc(-n3nccn3)c(Cl)c2)c2cnc3cc(F)nn3c21] | 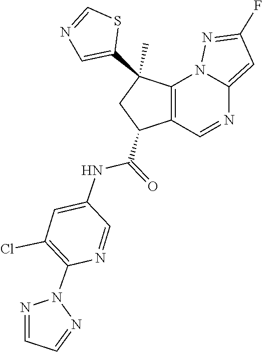 [SMILES: C[C@@]1(c2cncs2)C[C@@H](C(=O)Nc2cnc(-n3nccn3)c(Cl)c2)c2cnc3cc(F)nn3c21] |
| 38 (first eluting isomer)* 39 (second eluting isomer)* | 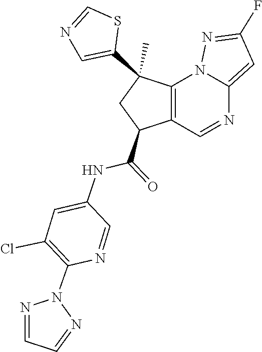 [SMILES: C[C@@]1(c2cncs2)C[C@H](C(=O)Nc2cnc(-n3nccn3)c(Cl)c2)c2cnc3cc(F)nn3c21] | 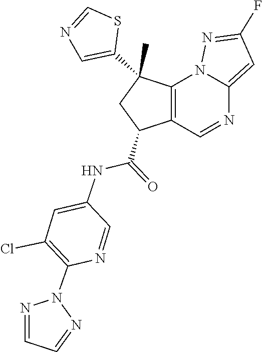 [SMILES: C[C@]1(c2cncs2)C[C@@H](C(=O)Nc2cnc(-n3nccn3)c(Cl)c2)c2cnc3cc(F)nn3c21] |
| 40 (second eluting isomer)* 41 (first eluting isomer)* | 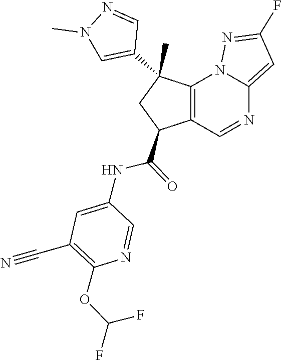 [SMILES: Cn1cc([C@@]2(C)C[C@H](C(=O)Nc3cnc(OC(F)F)c(C#N)c3)c3cnc4cc(F)nn4c32)cn1] | 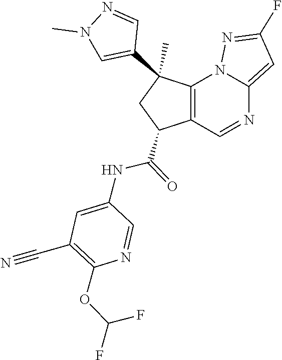 [SMILES: Cn1cc([C@]2(C)C[C@@H](C(=O)Nc3cnc(OC(F)F)c(C#N)c3)c3cnc4cc(F)nn4c32)cn1] |
| 42 (second eluting isomer)* 43 (first eluting isomer)* | 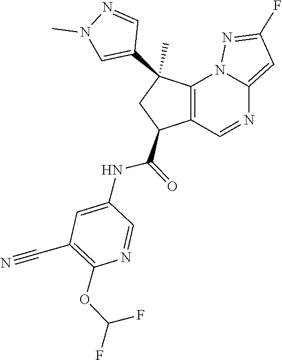 [SMILES: Cn1cc([C@]2(C)C[C@H](C(=O)Nc3cnc(OC(F)F)c(C#N)c3)c3cnc4cc(F)nn4c32)cn1] | 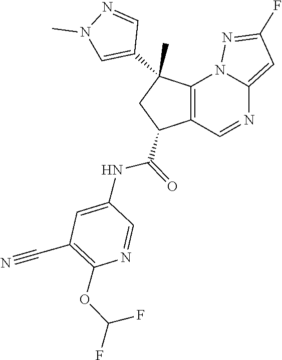 [SMILES: Cn1cc([C@@]2(C)C[C@@H](C(=O)Nc3cnc(OC(F)F)c(C#N)c3)c3cnc4cc(F)nn4c32)cn1] |
| 44 (second eluting isomer)* 45 (first eluting isomer)* | 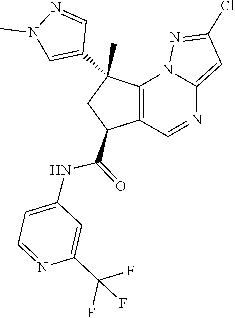 [SMILES: Cn1cc([C@@]2(C)C[C@H](C(=O)Nc3ccnc(C(F)(F)F)c3)c3cnc4cc(Cl)nn4c32)cn1] | 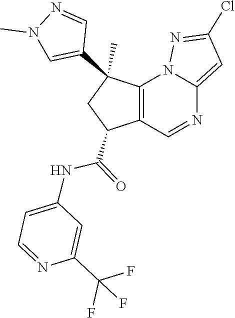 [SMILES: Cn1cc([C@]2(C)C[C@@H](C(=O)Nc3ccnc(C(F)(F)F)c3)c3cnc4cc(Cl)nn4c32)cn1] |
| 46 (first eluting isomer)* 47 (second eluting isomer)* | 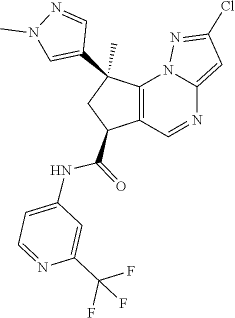 [SMILES: Cn1cc([C@]2(C)C[C@H](C(=O)Nc3ccnc(C(F)(F)F)c3)c3cnc4cc(Cl)nn4c32)cn1] | 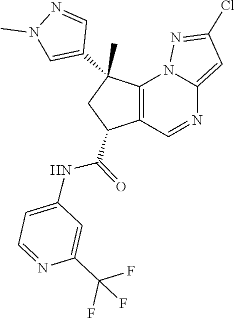 [SMILES: Cn1cc([C@@]2(C)C[C@@H](C(=O)Nc3ccnc(C(F)(F)F)c3)c3cnc4cc(Cl)nn4c32)cn1] |
| 48 (second eluting isomer)* 49 (first eluting isomer)* | 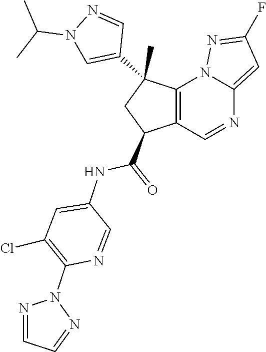 [SMILES: CC(C)n1cc([C@@]2(C)C[C@H](C(=O)Nc3cnc(-n4nccn4)c(Cl)c3)c3cnc4cc(F)nn4c32)cn1] | 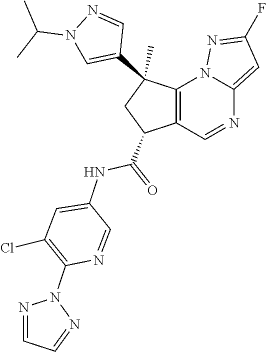 [SMILES: CC(C)n1cc([C@]2(C)C[C@@H](C(=O)Nc3cnc(-n4nccn4)c(Cl)c3)c3cnc4cc(F)nn4c32)cn1] |
| 50 (second eluting isomer)* 51 (first eluting isomer)* | 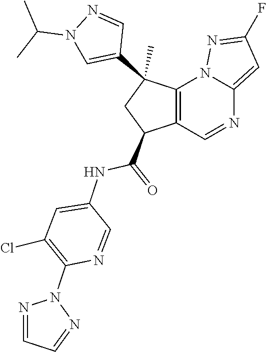 [SMILES: CC(C)n1cc([C@]2(C)C[C@H](C(=O)Nc3cnc(-n4nccn4)c(Cl)c3)c3cnc4cc(F)nn4c32)cn1] | 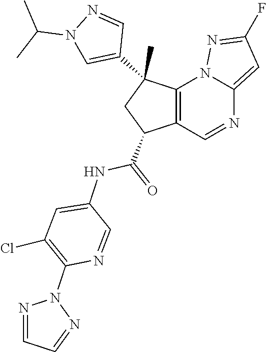 [SMILES: CC(C)n1cc([C@@]2(C)C[C@@H](C(=O)Nc3cnc(-n4nccn4)c(Cl)c3)c3cnc4cc(F)nn4c32)cn1] |
| 52 (second eluting isomer)* 53 (first eluting isomer)* | 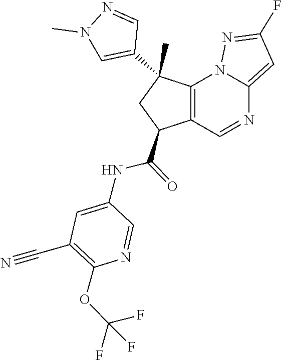 [SMILES: Cn1cc([C@@]2(C)C[C@H](C(=O)Nc3cnc(OC(F)(F)F)c(C#N)c3)c3cnc4cc(F)nn4c32)cn1] | 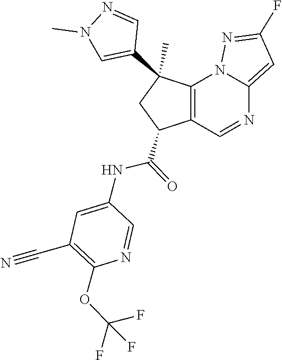 [SMILES: Cn1cc([C@]2(C)C[C@@H](C(=O)Nc3cnc(OC(F)(F)F)c(C#N)c3)c3cnc4cc(F)nn4c32)cn1] |
| 54 (second eluting isomer)* 55 (first eluting isomer)* | 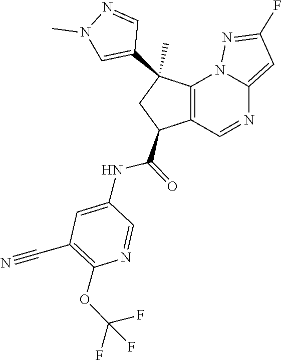 [SMILES: Cn1cc([C@]2(C)C[C@H](C(=O)Nc3cnc(OC(F)(F)F)c(C#N)c3)c3cnc4cc(F)nn4c32)cn1] | 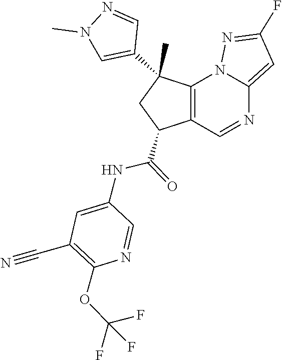 [SMILES: Cn1cc([C@@]2(C)C[C@@H](C(=O)Nc3cnc(OC(F)(F)F)c(C#N)c3)c3cnc4cc(F)nn4c32)cn1] |
| 56 (second eluting isomer)* 57 (first eluting isomer)* | 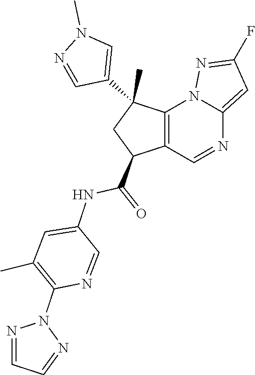 [SMILES: Cc1cc(NC(=O)[C@H]2C[C@](C)(c3cnn(C)c3)c3c2cnc2cc(F)nn32)cnc1-n1nccn1] | 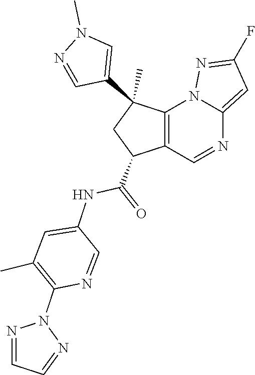 [SMILES: Cc1cc(NC(=O)[C@@H]2C[C@@](C)(c3cnn(C)c3)c3c2cnc2cc(F)nn32)cnc1-n1nccn1] |
| 58 (second eluting isomer)* 59 (first eluting isomer)* | 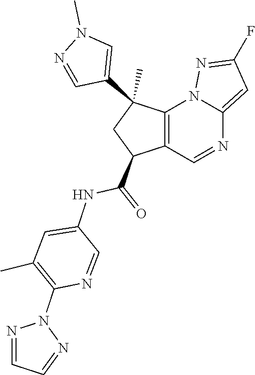 [SMILES: Cc1cc(NC(=O)[C@H]2C[C@@](C)(c3cnn(C)c3)c3c2cnc2cc(F)nn32)cnc1-n1nccn1] | 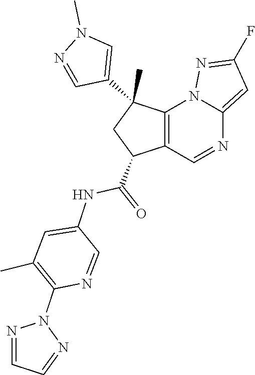 [SMILES: Cc1cc(NC(=O)[C@@H]2C[C@](C)(c3cnn(C)c3)c3c2cnc2cc(F)nn32)cnc1-n1nccn1] |
| 60 (second eluting isomer)* 61 (first eluting isomer)* | 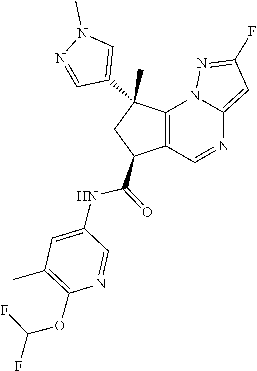 [SMILES: Cc1cc(NC(=O)[C@H]2C[C@](C)(c3cnn(C)c3)c3c2cnc2cc(F)nn32)cnc1OC(F)F] | 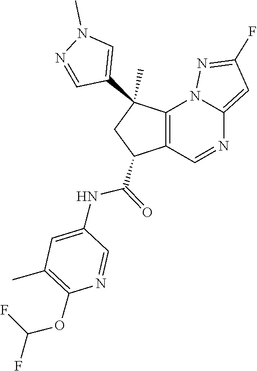 [SMILES: Cc1cc(NC(=O)[C@@H]2C[C@@](C)(c3cnn(C)c3)c3c2cnc2cc(F)nn32)cnc1OC(F)F] |
| 62 (first eluting isomer)* 63 (second eluting isomer)* | 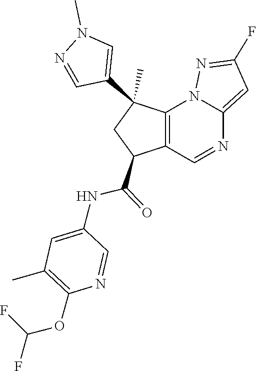 [SMILES: Cc1cc(NC(=O)[C@H]2C[C@@](C)(c3cnn(C)c3)c3c2cnc2cc(F)nn32)cnc1OC(F)F] | 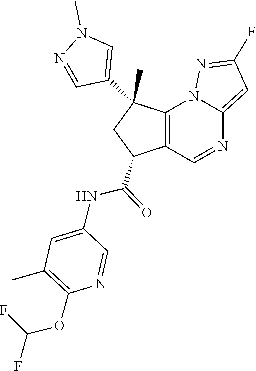 [SMILES: Cc1cc(NC(=O)[C@@H]2C[C@](C)(c3cnn(C)c3)c3c2cnc2cc(F)nn32)cnc1OC(F)F] |
| 64 (second eluting isomer)* 65 (first eluting isomer)* | 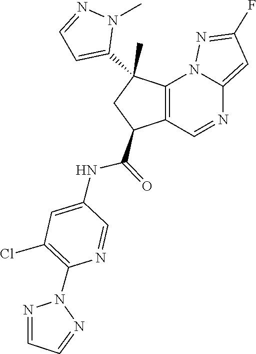 [SMILES: Cn1nccc1[C@@]1(C)C[C@H](C(=O)Nc2cnc(-n3nccn3)c(Cl)c2)c2cnc3cc(F)nn3c21] | 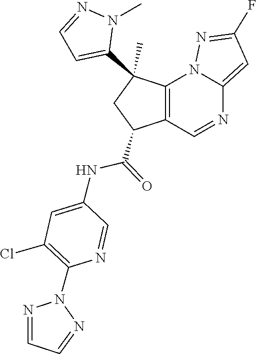 [SMILES: Cn1nccc1[C@]1(C)C[C@@H](C(=O)Nc2cnc(-n3nccn3)c(Cl)c2)c2cnc3cc(F)nn3c21] |
| 66 (second eluting isomer)* 67 (first eluting isomer)* | 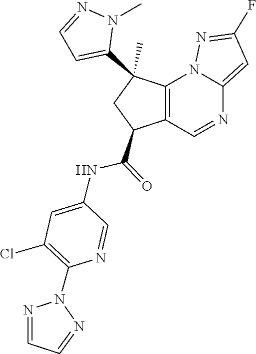 [SMILES: Cn1nccc1[C@]1(C)C[C@H](C(=O)Nc2cnc(-n3nccn3)c(Cl)c2)c2cnc3cc(F)nn3c21] | 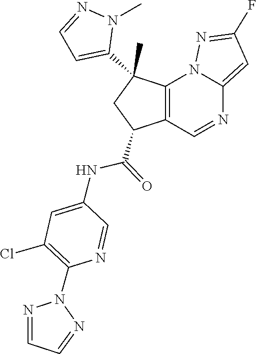 [SMILES: Cn1nccc1[C@@]1(C)C[C@@H](C(=O)Nc2cnc(-n3nccn3)c(Cl)c2)c2cnc3cc(F)nn3c21] |
| 68 (second eluting isomer)* 69 (first eluting isomer)* | 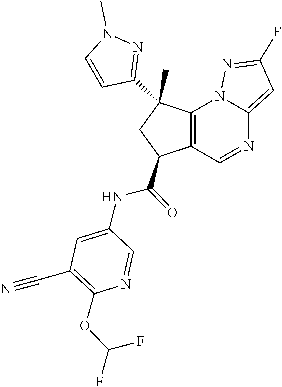 [SMILES: Cn1ccc([C@@]2(C)C[C@H](C(=O)Nc3cnc(OC(F)F)c(C#N)c3)c3cnc4cc(F)nn4c32)n1] | 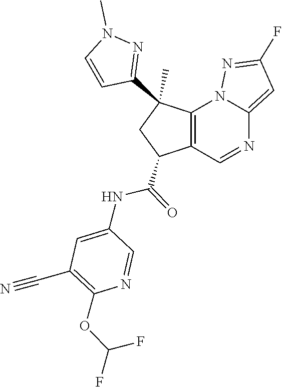 [SMILES: Cn1ccc([C@]2(C)C[C@@H](C(=O)Nc3cnc(OC(F)F)c(C#N)c3)c3cnc4cc(F)nn4c32)n1] |
| 70 (second eluting isomer)* 71 (first eluting isomer)* | 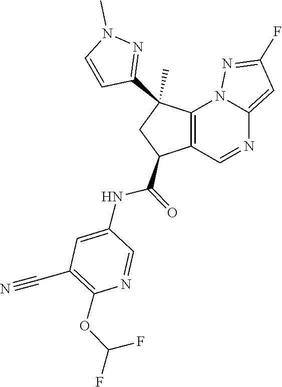 [SMILES: Cn1ccc([C@]2(C)C[C@H](C(=O)Nc3cnc(OC(F)F)c(C#N)c3)c3cnc4cc(F)nn4c32)n1] | 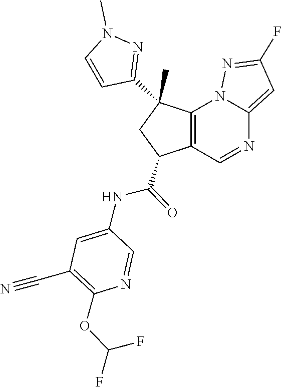 [SMILES: Cn1ccc([C@@]2(C)C[C@@H](C(=O)Nc3cnc(OC(F)F)c(C#N)c3)c3cnc4cc(F)nn4c32)n1] |
| 72 (second eluting isomer)* 73 (first eluting isomer)* | 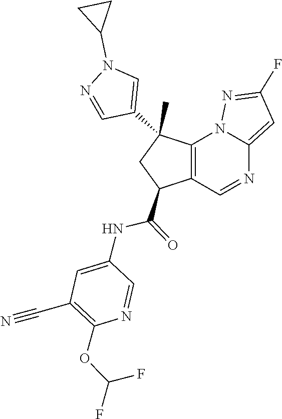 [SMILES: C[C@]1(c2cnn(C3CC3)c2)C[C@H](C(=O)Nc2cnc(OC(F)F)c(C#N)c2)c2cnc3cc(F)nn3c21] | 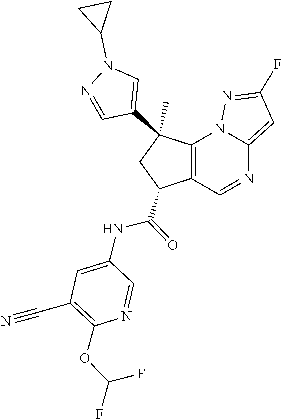 [SMILES: C[C@@]1(c2cnn(C3CC3)c2)C[C@@H](C(=O)Nc2cnc(OC(F)F)c(C#N)c2)c2cnc3cc(F)nn3c21] |
| 74 (first eluting isomer)* 75 (second eluting isomer)* | 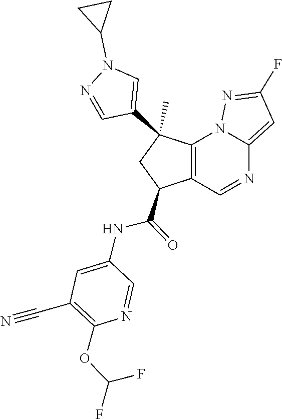 [SMILES: C[C@@]1(c2cnn(C3CC3)c2)C[C@H](C(=O)Nc2cnc(OC(F)F)c(C#N)c2)c2cnc3cc(F)nn3c21] | 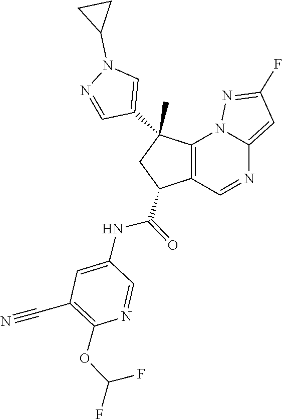 [SMILES: C[C@]1(c2cnn(C3CC3)c2)C[C@@H](C(=O)Nc2cnc(OC(F)F)c(C#N)c2)c2cnc3cc(F)nn3c21] |
| 76 (second eluting isomer)* 77 (first eluting isomer)* | 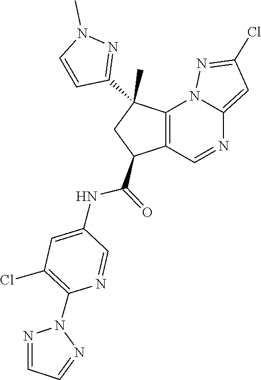 [SMILES: Cn1ccc([C@@]2(C)C[C@H](C(=O)Nc3cnc(-n4nccn4)c(Cl)c3)c3cnc4cc(Cl)nn4c32)n1] | 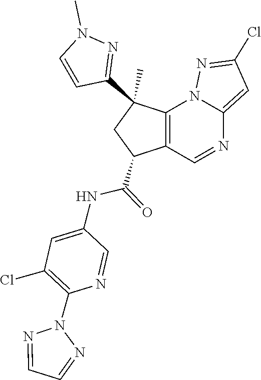 [SMILES: Cn1ccc([C@]2(C)C[C@@H](C(=O)Nc3cnc(-n4nccn4)c(Cl)c3)c3cnc4cc(Cl)nn4c32)n1] |
| 78 (first eluting isomer)* 79 (second eluting isomer)* | 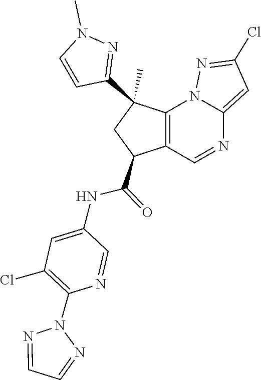 [SMILES: Cn1ccc([C@]2(C)C[C@H](C(=O)Nc3cnc(-n4nccn4)c(Cl)c3)c3cnc4cc(Cl)nn4c32)n1] | 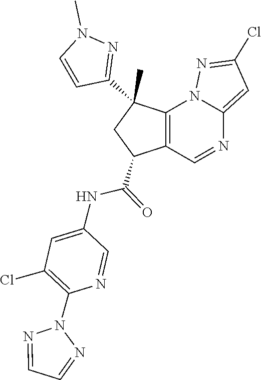 [SMILES: Cn1ccc([C@@]2(C)C[C@@H](C(=O)Nc3cnc(-n4nccn4)c(Cl)c3)c3cnc4cc(Cl)nn4c32)n1] |
| 80 (second eluting isomer)* 81 (first eluting isomer)* | 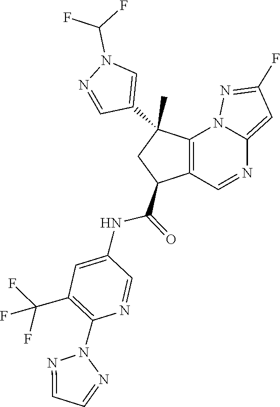 [SMILES: C[C@]1(c2cnn(C(F)F)c2)C[C@H](C(=O)Nc2cnc(-n3nccn3)c(C(F)(F)F)c2)c2cnc3cc(F)nn3c21] | 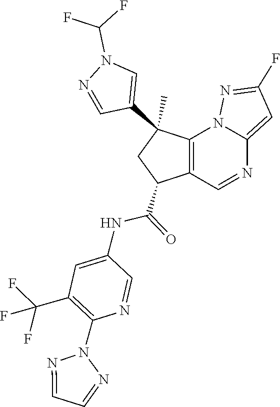 [SMILES: C[C@@]1(c2cnn(C(F)F)c2)C[C@@H](C(=O)Nc2cnc(-n3nccn3)c(C(F)(F)F)c2)c2cnc3cc(F)nn3c21] |
| 82 (second eluting isomer)* 83 (first eluting isomer)* | 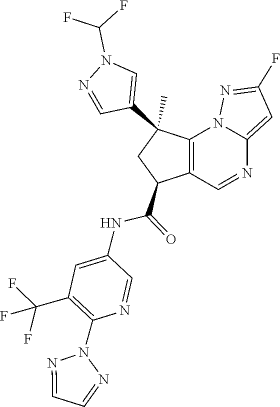 [SMILES: C[C@@]1(c2cnn(C(F)F)c2)C[C@H](C(=O)Nc2cnc(-n3nccn3)c(C(F)(F)F)c2)c2cnc3cc(F)nn3c21] | 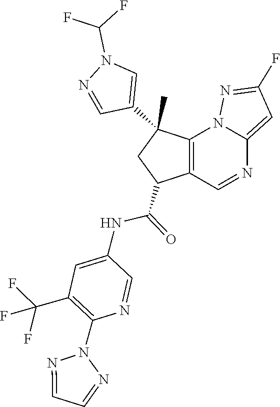 |
| 84 (second eluting isomer)* 85 (first eluting isomer)* | 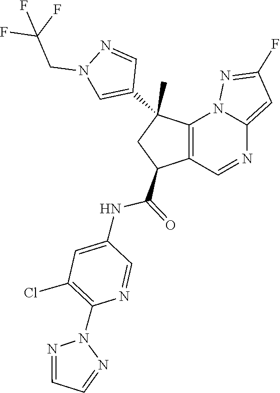 [SMILES: C[C@]1(c2cnn(CC(F)(F)F)c2)C[C@H](C(=O)Nc2cnc(-n3nccn3)c(Cl)c2)c2cnc3cc(F)nn3c21] | 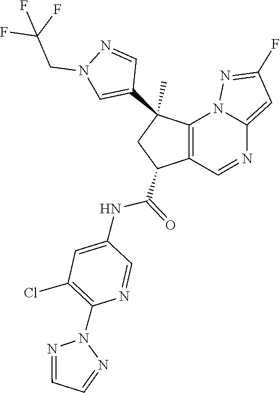 [SMILES: C[C@@]1(c2cnn(CC(F)(F)F)c2)C[C@@H](C(=O)Nc2cnc(-n3nccn3)c(Cl)c2)c2cnc3cc(F)nn3c21] |
| 86 (second eluting isomer)* 87 (first eluting isomer)* | 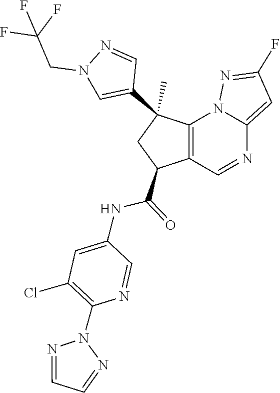 [SMILES: C[C@@]1(c2cnn(CC(F)(F)F)c2)C[C@H](C(=O)Nc2cnc(-n3nccn3)c(Cl)c2)c2cnc3cc(F)nn3c21] | 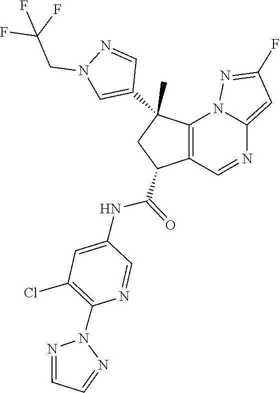 [SMILES: C[C@]1(c2cnn(CC(F)(F)F)c2)C[C@@H](C(=O)Nc2cnc(-n3nccn3)c(Cl)c2)c2cnc3cc(F)nn3c21] |
| 88 (second eluting isomer)* 89 (first eluting isomer)* | 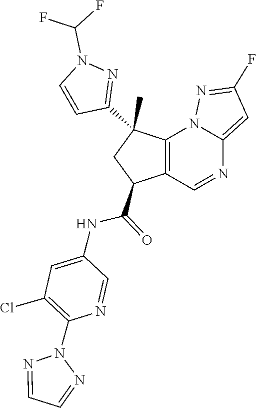 [SMILES: C[C@]1(c2ccn(C(F)F)n2)C[C@H](C(=O)Nc2cnc(-n3nccn3)c(Cl)c2)c2cnc3cc(F)nn3c21] | 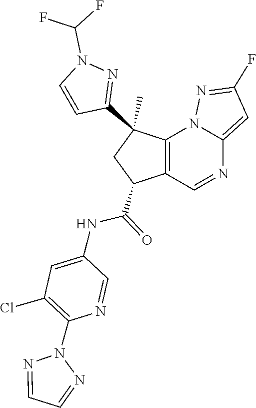 [SMILES: C[C@@]1(c2ccn(C(F)F)n2)C[C@@H](C(=O)Nc2cnc(-n3nccn3)c(Cl)c2)c2cnc3cc(F)nn3c21] |
| 90 (second eluting isomer)* 91 (first eluting isomer)* | 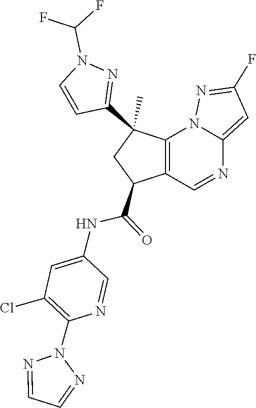 [SMILES: C[C@@]1(c2ccn(C(F)F)n2)C[C@H](C(=O)Nc2cnc(-n3nccn3)c(Cl)c2)c2cnc3cc(F)nn3c21] | 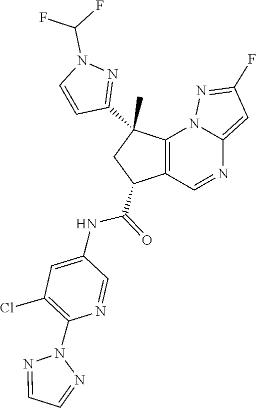 [SMILES: C[C@]1(c2ccn(C(F)F)n2)C[C@@H](C(=O)Nc2cnc(-n3nccn3)c(Cl)c2)c2cnc3cc(F)nn3c21] |
| 92 (second eluting isomer)* 93 (first eluting isomer)* | 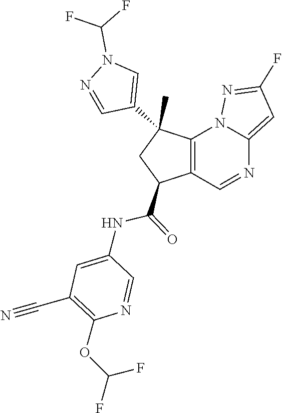 [SMILES: C[C@]1(c2cnn(C(F)F)c2)C[C@H](C(=O)Nc2cnc(OC(F)F)c(C#N)c2)c2cnc3cc(F)nn3c21] | 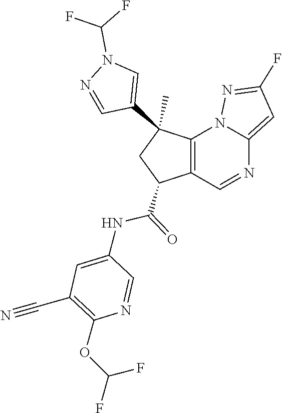 [SMILES: C[C@@]1(c2cnn(C(F)F)c2)C[C@@H](C(=O)Nc2cnc(OC(F)F)c(C#N)c2)c2cnc3cc(F)nn3c21] |
| 94 (second eluting isomer)* 95 (first eluting isomer)* | 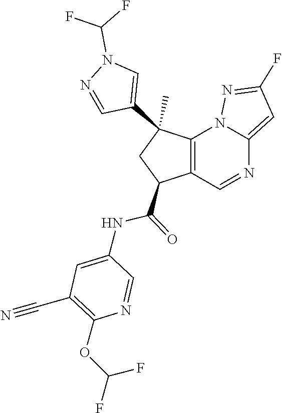 [SMILES: C[C@@]1(c2cnn(C(F)F)c2)C[C@H](C(=O)Nc2cnc(OC(F)F)c(C#N)c2)c2cnc3cc(F)nn3c21] | 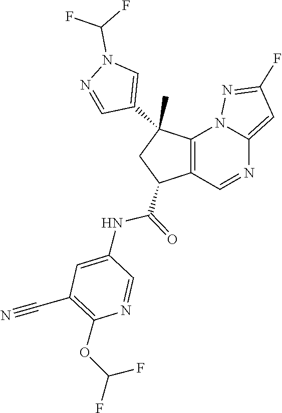 [SMILES: C[C@]1(c2cnn(C(F)F)c2)C[C@@H](C(=O)Nc2cnc(OC(F)F)c(C#N)c2)c2cnc3cc(F)nn3c21] |
| 96 and 97 | 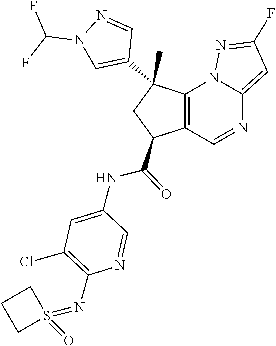 [SMILES: C[C@]1(c2cnn(C(F)F)c2)C[C@H](C(=O)Nc2cnc(N=S3(=O)CCC3)c(Cl)c2)c2cnc3cc(F)nn3c21] | 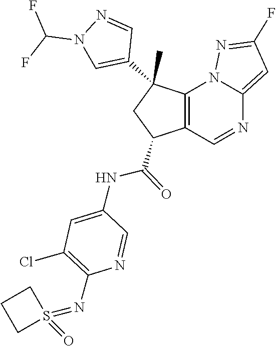 [SMILES: C[C@]1(c2cnn(C(F)F)c2)C[C@@H](C(=O)Nc2cnc(N=S3(=O)CCC3)c(Cl)c2)c2cnc3cc(F)nn3c21] |
| 98 (second eluting isomer)* 99 (first eluting isomer)* | 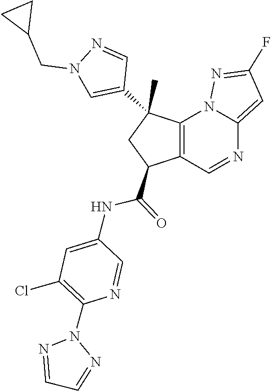 [SMILES: C[C@]1(c2cnn(CC3CC3)c2)C[C@H](C(=O)Nc2cnc(-n3nccn3)c(Cl)c2)c2cnc3cc(F)nn3c21] | 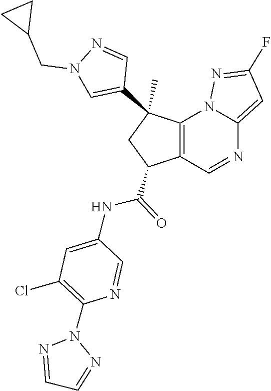 [SMILES: C[C@@]1(c2cnn(CC3CC3)c2)C[C@@H](C(=O)Nc2cnc(-n3nccn3)c(Cl)c2)c2cnc3cc(F)nn3c21] |
| 100 (second eluting isomer)* 101 (first eluting isomer)* | 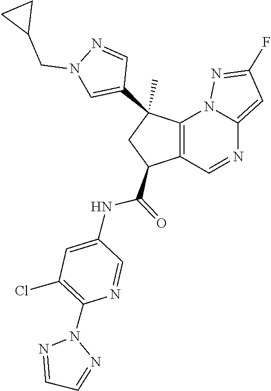 [SMILES: C[C@@]1(c2cnn(CC3CC3)c2)C[C@H](C(=O)Nc2cnc(-n3nccn3)c(Cl)c2)c2cnc3cc(F)nn3c21] | 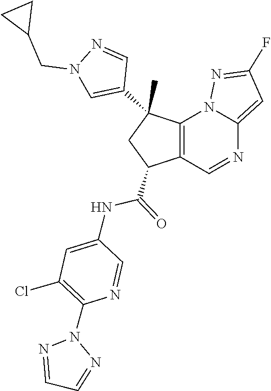 [SMILES: C[C@]1(c2cnn(CC3CC3)c2)C[C@@H](C(=O)Nc2cnc(-n3nccn3)c(Cl)c2)c2cnc3cc(F)nn3c21] |
| 102 (second eluting isomer)* 103 (first eluting isomer)* | 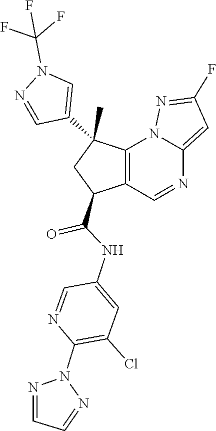 [SMILES: C[C@]1(c2cnn(C(F)(F)F)c2)C[C@H](C(=O)Nc2cnc(-n3nccn3)c(Cl)c2)c2cnc3cc(F)nn3c21] | 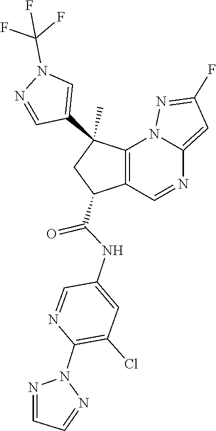 [SMILES: C[C@@]1(c2cnn(C(F)(F)F)c2)C[C@@H](C(=O)Nc2cnc(-n3nccn3)c(Cl)c2)c2cnc3cc(F)nn3c21] |
| 104 (second eluting isomer)* 105 (first eluting isomer)* | 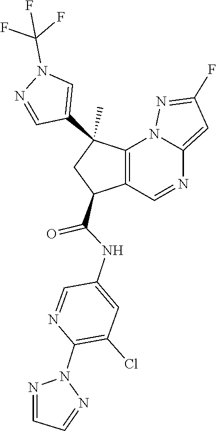 [SMILES: C[C@@]1(c2cnn(C(F)(F)F)c2)C[C@H](C(=O)Nc2cnc(-n3nccn3)c(Cl)c2)c2cnc3cc(F)nn3c21] | 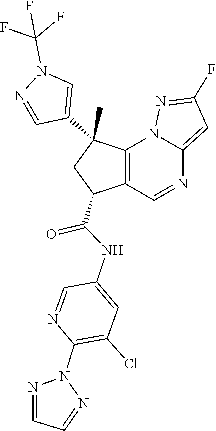 [SMILES: C[C@]1(c2cnn(C(F)(F)F)c2)C[C@@H](C(=O)Nc2cnc(-n3nccn3)c(Cl)c2)c2cnc3cc(F)nn3c21] |
| 106, 107, 108 and 109 (multiple separation methods)* | 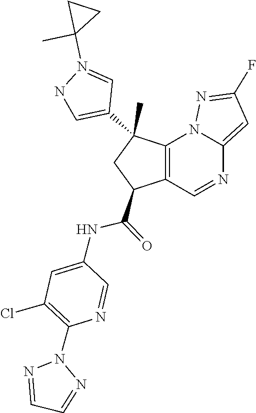 | 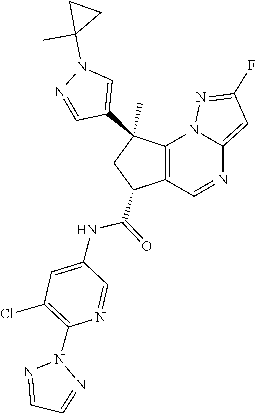 [SMILES: CC1(n2cc([C@]3(C)C[C@@H](C(=O)Nc4cnc(-n5nccn5)c(Cl)c4)c4cnc5cc(F)nn5c43)cn2)CC1] |
| | 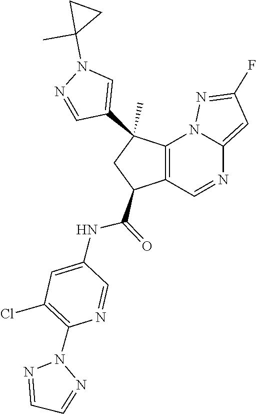 [SMILES: CC1(n2cc([C@]3(C)C[C@H](C(=O)Nc4cnc(-n5nccn5)c(Cl)c4)c4cnc5cc(F)nn5c43)cn2)CC1] | 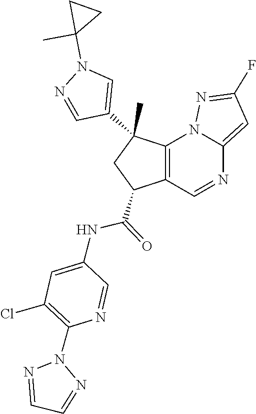 [SMILES: CC1(n2cc([C@@]3(C)C[C@@H](C(=O)Nc4cnc(-n5nccn5)c(Cl)c4)c4cnc5cc(F)nn5c43)cn2)CC1] |
| 110 (second eluting isomer)* 111 (first eluting isomer)* | 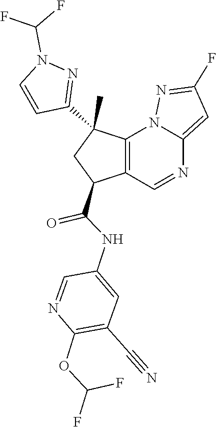 [SMILES: C[C@]1(c2ccn(C(F)F)n2)C[C@H](C(=O)Nc2cnc(OC(F)F)c(C#N)c2)c2cnc3cc(F)nn3c21] | 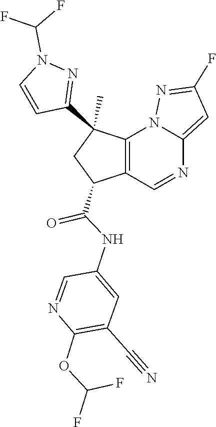 [SMILES: C[C@@]1(c2ccn(C(F)F)n2)C[C@@H](C(=O)Nc2cnc(OC(F)F)c(C#N)c2)c2cnc3cc(F)nn3c21] |
| 112 (second eluting isomer)* 113 (first eluting isomer)* | 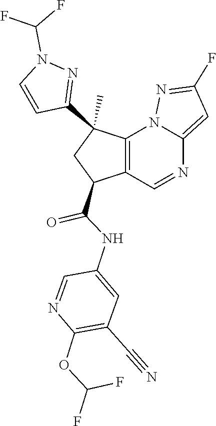 [SMILES: C[C@@]1(c2ccn(C(F)F)n2)C[C@H](C(=O)Nc2cnc(OC(F)F)c(C#N)c2)c2cnc3cc(F)nn3c21] | 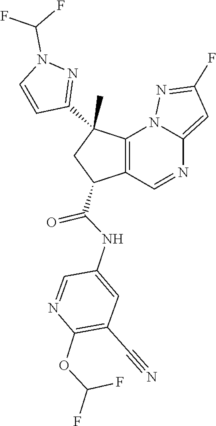 [SMILES: C[C@]1(c2ccn(C(F)F)n2)C[C@@H](C(=O)Nc2cnc(OC(F)F)c(C#N)c2)c2cnc3cc(F)nn3c21] |
| 114 and 115 | 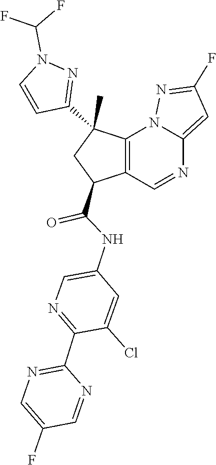 [SMILES: C[C@]1(c2ccn(C(F)F)n2)C[C@H](C(=O)Nc2cnc(-c3ncc(F)cn3)c(Cl)c2)c2cnc3cc(F)nn3c21] | 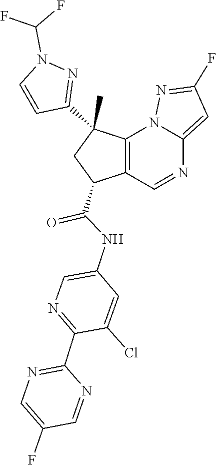 [SMILES: C[C@]1(c2ccn(C(F)F)n2)C[C@@H](C(=O)Nc2cnc(-c3ncc(F)cn3)c(Cl)c2)c2cnc3cc(F)nn3c21] |
| 116 and 117 | 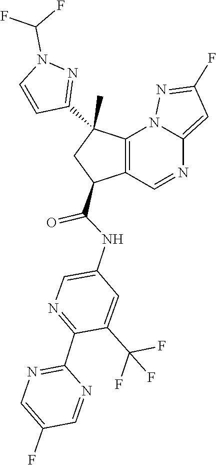 [SMILES: C[C@]1(c2ccn(C(F)F)n2)C[C@H](C(=O)Nc2cnc(-c3ncc(F)cn3)c(C(F)(F)F)c2)c2cnc3cc(F)nn3c21] | 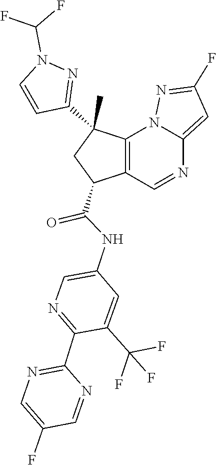 [SMILES: C[C@]1(c2ccn(C(F)F)n2)C[C@@H](C(=O)Nc2cnc(-c3ncc(F)cn3)c(C(F)(F)F)c2)c2cnc3cc(F)nn3c21] |
| 118 (first eluting isomer)* 119 (second eluting isomer)* | 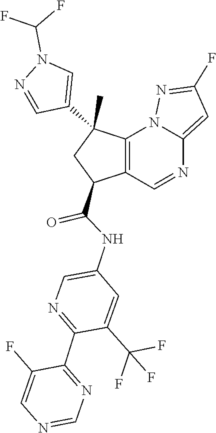 [SMILES: C[C@]1(c2cnn(C(F)F)c2)C[C@H](C(=O)Nc2cnc(-c3ncncc3F)c(C(F)(F)F)c2)c2cnc3cc(F)nn3c21] | 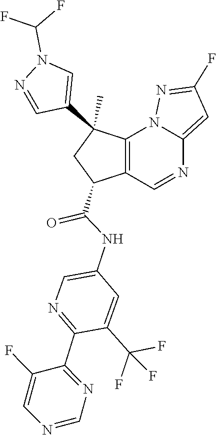 [SMILES: C[C@@]1(c2cnn(C(F)F)c2)C[C@@H](C(=O)Nc2cnc(-c3ncncc3F)c(C(F)(F)F)c2)c2cnc3cc(F)nn3c21] |
| 120 (first eluting isomer)* 121 (second eluting isomer)* | 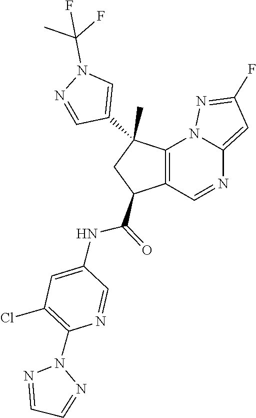 [SMILES: CC(F)(F)n1cc([C@@]2(C)C[C@H](C(=O)Nc3cnc(-n4nccn4)c(Cl)c3)c3cnc4cc(F)nn4c32)cn1] | 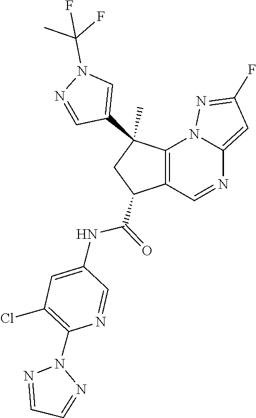 [SMILES: CC(F)(F)n1cc([C@]2(C)C[C@@H](C(=O)Nc3cnc(-n4nccn4)c(Cl)c3)c3cnc4cc(F)nn4c32)cn1] |
| 122 (first eluting isomer)* 123 (second eluting isomer)* | 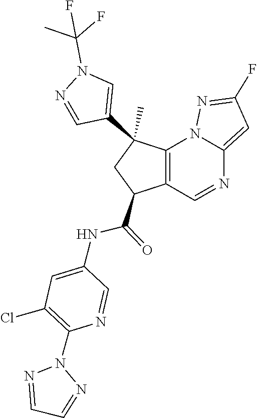 [SMILES: CC(F)(F)n1cc([C@]2(C)C[C@H](C(=O)Nc3cnc(-n4nccn4)c(Cl)c3)c3cnc4cc(F)nn4c32)cn1] | 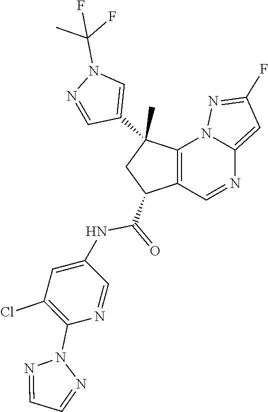 [SMILES: CC(F)(F)n1cc([C@@]2(C)C[C@@H](C(=O)Nc3cnc(-n4nccn4)c(Cl)c3)c3cnc4cc(F)nn4c32)cn1] |
| 124 (first eluting isomer)* 125 (second eluting isomer)* | 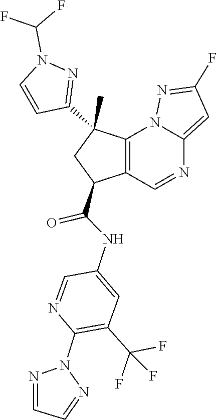 [SMILES: C[C@]1(c2ccn(C(F)F)n2)C[C@H](C(=O)Nc2cnc(-n3nccn3)c(C(F)(F)F)c2)c2cnc3cc(F)nn3c21] | 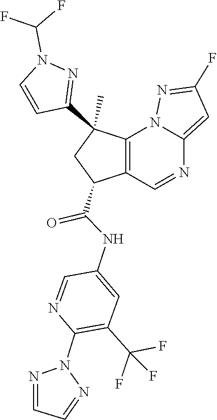 [SMILES: C[C@@]1(c2ccn(C(F)F)n2)C[C@@H](C(=O)Nc2cnc(-n3nccn3)c(C(F)(F)F)c2)c2cnc3cc(F)nn3c21] |
| 126 (second eluting isomer)* 127 (first eluting isomer)* | 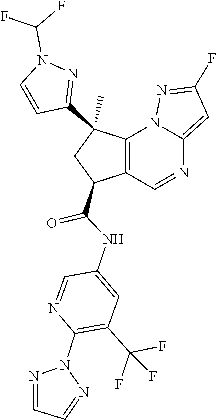 [SMILES: C[C@@]1(c2ccn(C(F)F)n2)C[C@H](C(=O)Nc2cnc(-n3nccn3)c(C(F)(F)F)c2)c2cnc3cc(F)nn3c21] | 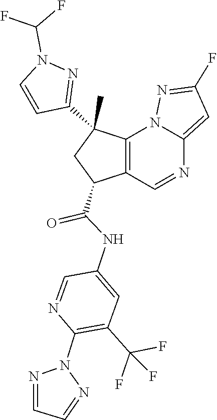 [SMILES: C[C@]1(c2ccn(C(F)F)n2)C[C@@H](C(=O)Nc2cnc(-n3nccn3)c(C(F)(F)F)c2)c2cnc3cc(F)nn3c21] |
| 128 (second eluting isomer)* 129 (first eluting isomer)* | 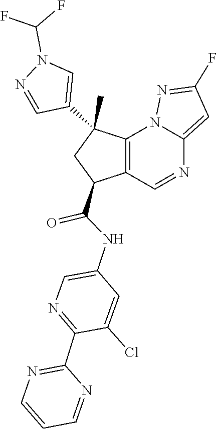 [SMILES: C[C@]1(c2cnn(C(F)F)c2)C[C@H](C(=O)Nc2cnc(-c3ncccn3)c(Cl)c2)c2cnc3cc(F)nn3c21] | 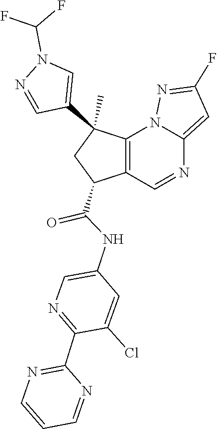 [SMILES: C[C@@]1(c2cnn(C(F)F)c2)C[C@@H](C(=O)Nc2cnc(-c3ncccn3)c(Cl)c2)c2cnc3cc(F)nn3c21] |
| 130 (first eluting isomer)* 131 (second eluting isomer)* | 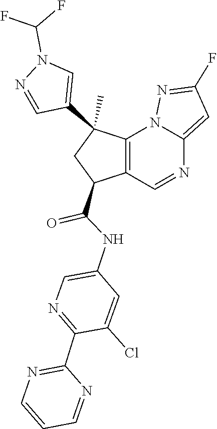 [SMILES: C[C@@]1(c2cnn(C(F)F)c2)C[C@H](C(=O)Nc2cnc(-c3ncccn3)c(Cl)c2)c2cnc3cc(F)nn3c21] | 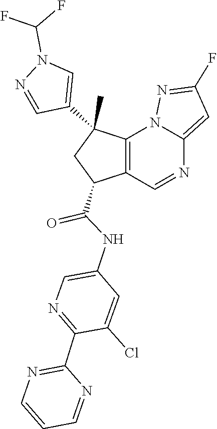 [SMILES: C[C@]1(c2cnn(C(F)F)c2)C[C@@H](C(=O)Nc2cnc(-c3ncccn3)c(Cl)c2)c2cnc3cc(F)nn3c21] |
| 132 and 133 | 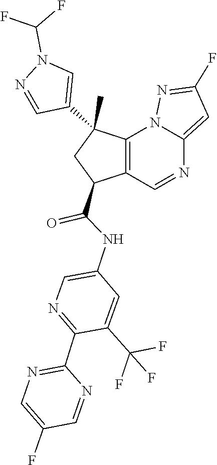 [SMILES: C[C@]1(c2cnn(C(F)F)c2)C[C@H](C(=O)Nc2cnc(-c3ncc(F)cn3)c(C(F)(F)F)c2)c2cnc3cc(F)nn3c21] | 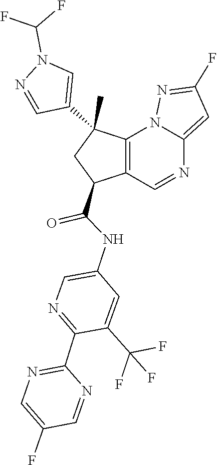 [SMILES: C[C@]1(c2cnn(C(F)F)c2)C[C@H](C(=O)Nc2cnc(-c3ncc(F)cn3)c(C(F)(F)F)c2)c2cnc3cc(F)nn3c21] |
| 134 and 135 | 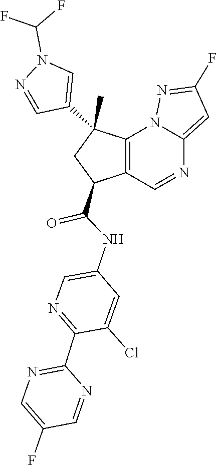 [SMILES: C[C@]1(c2cnn(C(F)F)c2)C[C@H](C(=O)Nc2cnc(-c3ncc(F)cn3)c(Cl)c2)c2cnc3cc(F)nn3c21] | 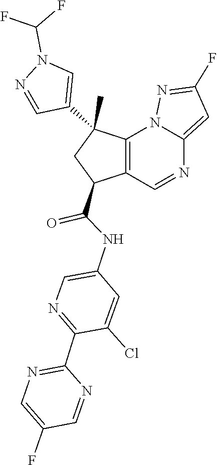 [SMILES: C[C@]1(c2cnn(C(F)F)c2)C[C@H](C(=O)Nc2cnc(-c3ncc(F)cn3)c(Cl)c2)c2cnc3cc(F)nn3c21] |
| 136 (second eluting isomer)* 137 (first eluting isomer)* | 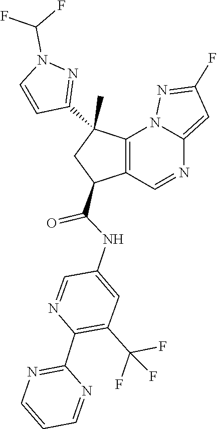 [SMILES: C[C@]1(c2ccn(C(F)F)n2)C[C@H](C(=O)Nc2cnc(-c3ncccn3)c(C(F)(F)F)c2)c2cnc3cc(F)nn3c21] | 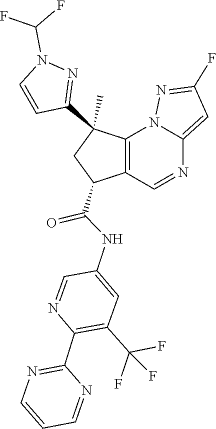 [SMILES: C[C@@]1(c2ccn(C(F)F)n2)C[C@@H](C(=O)Nc2cnc(-c3ncccn3)c(C(F)(F)F)c2)c2cnc3cc(F)nn3c21] |
| 138 (first eluting isomer)* 139 (second eluting isomer)* | 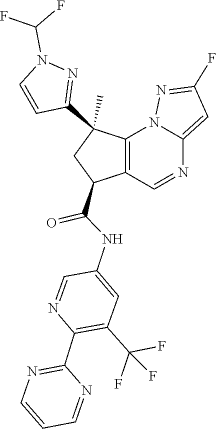 [SMILES: C[C@@]1(c2ccn(C(F)F)n2)C[C@H](C(=O)Nc2cnc(-c3ncccn3)c(C(F)(F)F)c2)c2cnc3cc(F)nn3c21] | 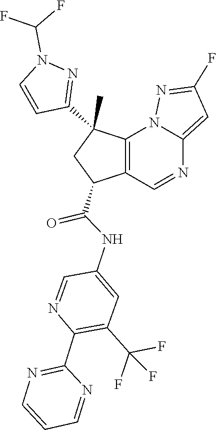 [SMILES: C[C@]1(c2ccn(C(F)F)n2)C[C@@H](C(=O)Nc2cnc(-c3ncccn3)c(C(F)(F)F)c2)c2cnc3cc(F)nn3c21] |
| 140 (second eluting isomer)* 141 (first eluting isomer)* | 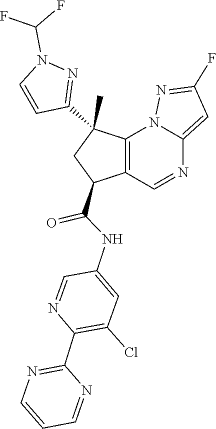 [SMILES: C[C@]1(c2ccn(C(F)F)n2)C[C@H](C(=O)Nc2cnc(-c3ncccn3)c(Cl)c2)c2cnc3cc(F)nn3c21] | 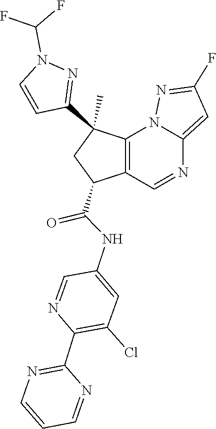 [SMILES: C[C@@]1(c2ccn(C(F)F)n2)C[C@@H](C(=O)Nc2cnc(-c3ncccn3)c(Cl)c2)c2cnc3cc(F)nn3c21] |
| 142 (second eluting isomer)* 143 (first eluting isomer)* | 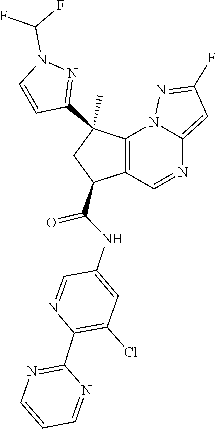 [SMILES: C[C@@]1(c2ccn(C(F)F)n2)C[C@H](C(=O)Nc2cnc(-c3ncccn3)c(Cl)c2)c2cnc3cc(F)nn3c21] | 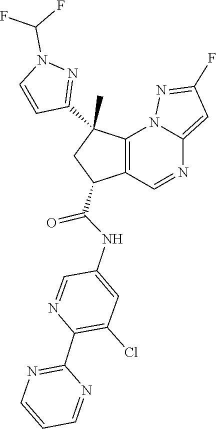 [SMILES: C[C@]1(c2ccn(C(F)F)n2)C[C@@H](C(=O)Nc2cnc(-c3ncccn3)c(Cl)c2)c2cnc3cc(F)nn3c21] |
| 144 (second eluting isomer)* 145 (first eluting isomer)* | 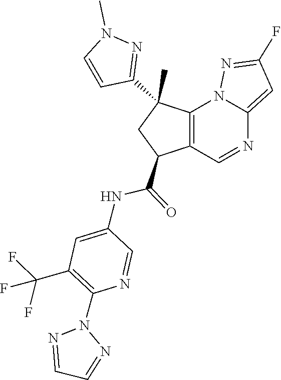 [SMILES: Cn1ccc([C@@]2(C)C[C@H](C(=O)Nc3cnc(-n4nccn4)c(C(F)(F)F)c3)c3cnc4cc(F)nn4c32)n1] | 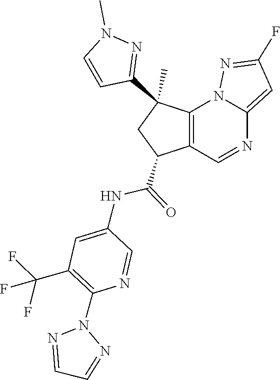 [SMILES: Cn1ccc([C@]2(C)C[C@@H](C(=O)Nc3cnc(-n4nccn4)c(C(F)(F)F)c3)c3cnc4cc(F)nn4c32)n1] |
| 146 (first eluting isomer)* 147 second eluting isomer)* | 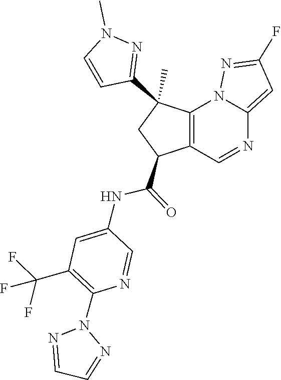 [SMILES: Cn1ccc([C@]2(C)C[C@H](C(=O)Nc3cnc(-n4nccn4)c(C(F)(F)F)c3)c3cnc4cc(F)nn4c32)n1] | 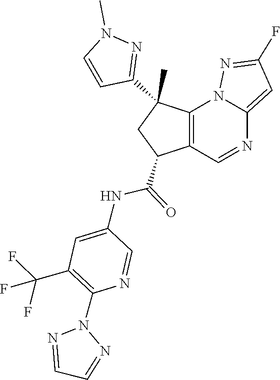 [SMILES: Cn1ccc([C@@]2(C)C[C@@H](C(=O)Nc3cnc(-n4nccn4)c(C(F)(F)F)c3)c3cnc4cc(F)nn4c32)n1] |
| 148 (second eluting isomer)* 149 (first eluting isomer)* | 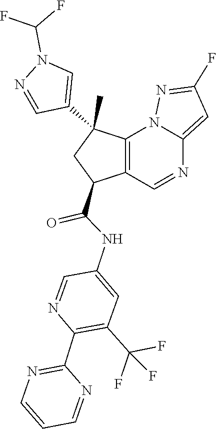 [SMILES: C[C@]1(c2cnn(C(F)F)c2)C[C@H](C(=O)Nc2cnc(-c3ncccn3)c(C(F)(F)F)c2)c2cnc3cc(F)nn3c21] | 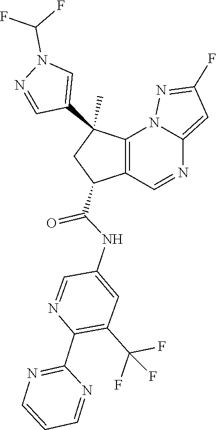 [SMILES: C[C@@]1(c2cnn(C(F)F)c2)C[C@@H](C(=O)Nc2cnc(-c3ncccn3)c(C(F)(F)F)c2)c2cnc3cc(F)nn3c21] |
| 150 (first eluting isomer)* 151 (second eluting isomer)* | 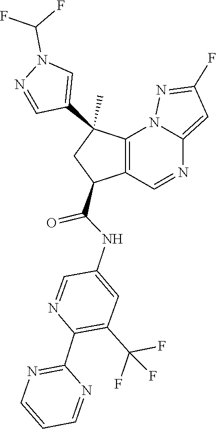 [SMILES: C[C@@]1(c2cnn(C(F)F)c2)C[C@H](C(=O)Nc2cnc(-c3ncccn3)c(C(F)(F)F)c2)c2cnc3cc(F)nn3c21] | 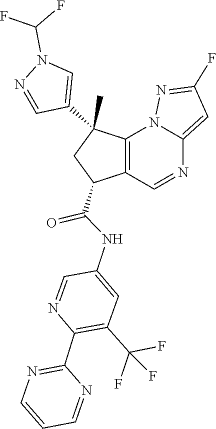 [SMILES: C[C@]1(c2cnn(C(F)F)c2)C[C@@H](C(=O)Nc2cnc(-c3ncccn3)c(C(F)(F)F)c2)c2cnc3cc(F)nn3c21] |
| 152 (second eluting isomer)* 153 (first eluting isomer)* | 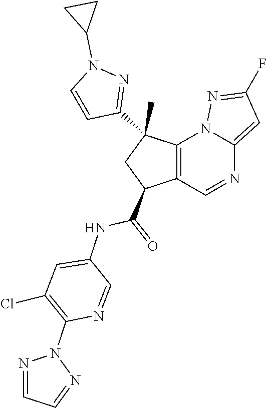 [SMILES: C[C@]1(c2ccn(C3CC3)n2)C[C@H](C(=O)Nc2cnc(-n3nccn3)c(Cl)c2)c2cnc3cc(F)nn3c21] | 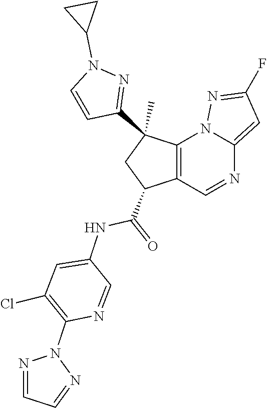 [SMILES: C[C@@]1(c2ccn(C3CC3)n2)C[C@@H](C(=O)Nc2cnc(-n3nccn3)c(Cl)c2)c2cnc3cc(F)nn3c21] |
| 154 (first eluting isomer)* 155 (second eluting isomer)* | 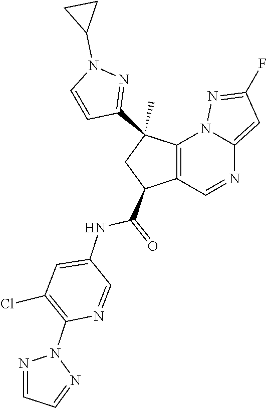 [SMILES: C[C@@]1(c2ccn(C3CC3)n2)C[C@H](C(=O)Nc2cnc(-n3nccn3)c(Cl)c2)c2cnc3cc(F)nn3c21] | 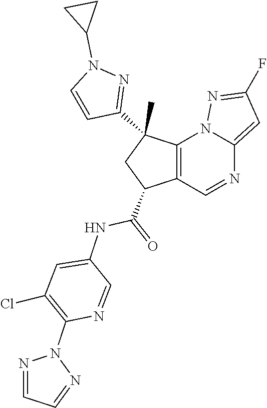 [SMILES: C[C@]1(c2ccn(C3CC3)n2)C[C@@H](C(=O)Nc2cnc(-n3nccn3)c(Cl)c2)c2cnc3cc(F)nn3c21] |
| 156 (second eluting isomer)* 157 (first eluting isomer)* | 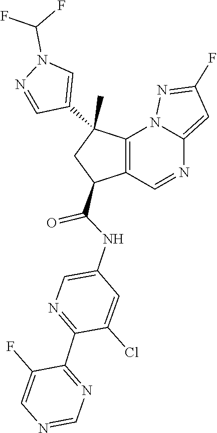 [SMILES: C[C@]1(c2cnn(C(F)F)c2)C[C@H](C(=O)Nc2cnc(-c3ncncc3F)c(Cl)c2)c2cnc3cc(F)nn3c21] | 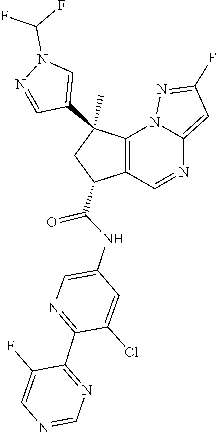 [SMILES: C[C@@]1(c2cnn(C(F)F)c2)C[C@@H](C(=O)Nc2cnc(-c3ncncc3F)c(Cl)c2)c2cnc3cc(F)nn3c21] |
| 158 (second eluting isomer)* 159 (first eluting isomer)* | 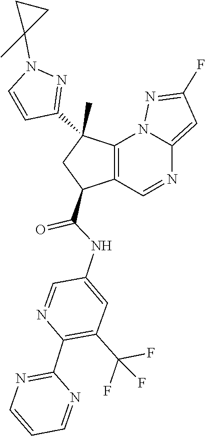 [SMILES: CC1(n2ccc([C@@]3(C)C[C@H](C(=O)Nc4cnc(-c5ncccn5)c(C(F)(F)F)c4)c4cnc5cc(F)nn5c43)n2)CC1] | 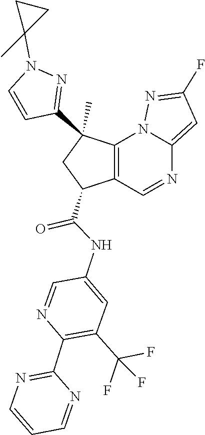 [SMILES: CC1(n2ccc([C@]3(C)C[C@@H](C(=O)Nc4cnc(-c5ncccn5)c(C(F)(F)F)c4)c4cnc5cc(F)nn5c43)n2)CC1] |
| 160 (first eluting isomer)* 161 (second eluting isomer)* | 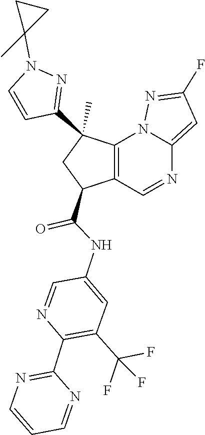 [SMILES: CC1(n2ccc([C@]3(C)C[C@H](C(=O)Nc4cnc(-c5ncccn5)c(C(F)(F)F)c4)c4cnc5cc(F)nn5c43)n2)CC1] | 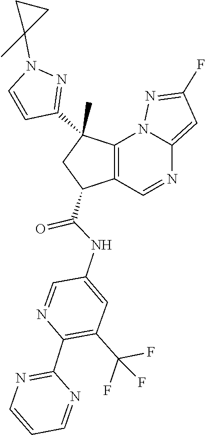 [SMILES: CC1(n2ccc([C@@]3(C)C[C@@H](C(=O)Nc4cnc(-c5ncccn5)c(C(F)(F)F)c4)c4cnc5cc(F)nn5c43)n2)CC1] |
| 162 and 163 | 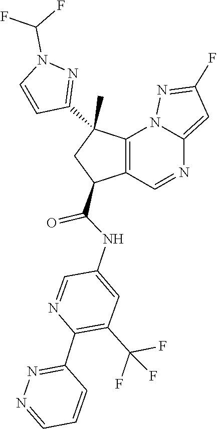 [SMILES: C[C@]1(c2ccn(C(F)F)n2)C[C@H](C(=O)Nc2cnc(-c3cccnn3)c(C(F)(F)F)c2)c2cnc3cc(F)nn3c21] | 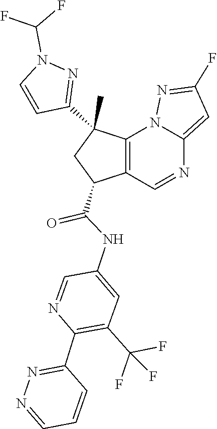 [SMILES: C[C@]1(c2ccn(C(F)F)n2)C[C@@H](C(=O)Nc2cnc(-c3cccnn3)c(C(F)(F)F)c2)c2cnc3cc(F)nn3c21] |
| 164 (second eluting isomer)* 165 (first eluting isomer)* | 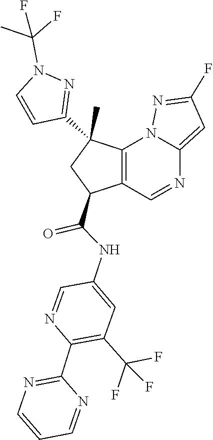 [SMILES: CC(F)(F)n1ccc([C@@]2(C)C[C@H](C(=O)Nc3cnc(-c4ncccn4)c(C(F)(F)F)c3)c3cnc4cc(F)nn4c32)n1] | 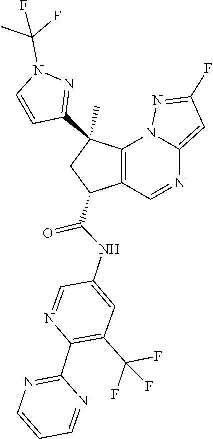 [SMILES: CC(F)(F)n1ccc([C@]2(C)C[C@@H](C(=O)Nc3cnc(-c4ncccn4)c(C(F)(F)F)c3)c3cnc4cc(F)nn4c32)n1] |
| 166 (second eluting isomer)* 167 (first eluting isomer)* | 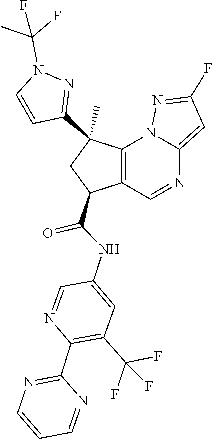 [SMILES: CC(F)(F)n1ccc([C@]2(C)C[C@H](C(=O)Nc3cnc(-c4ncccn4)c(C(F)(F)F)c3)c3cnc4cc(F)nn4c32)n1] | 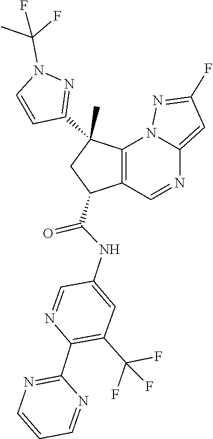 [SMILES: CC(F)(F)n1ccc([C@@]2(C)C[C@@H](C(=O)Nc3cnc(-c4ncccn4)c(C(F)(F)F)c3)c3cnc4cc(F)nn4c32)n1] |
| 168 (second eluting isomer)* 169 (first eluting isomer)* | 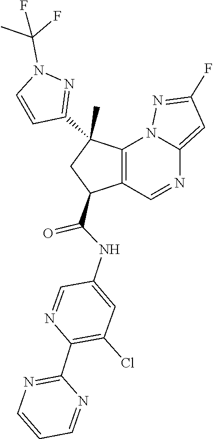 [SMILES: CC(F)(F)n1ccc([C@@]2(C)C[C@H](C(=O)Nc3cnc(-c4ncccn4)c(Cl)c3)c3cnc4cc(F)nn4c32)n1] | 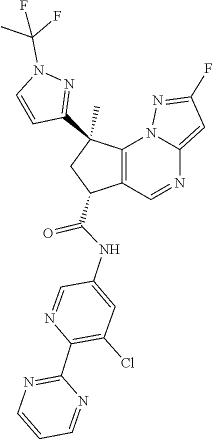 [SMILES: CC(F)(F)n1ccc([C@]2(C)C[C@@H](C(=O)Nc3cnc(-c4ncccn4)c(Cl)c3)c3cnc4cc(F)nn4c32)n1] |
| 170 | 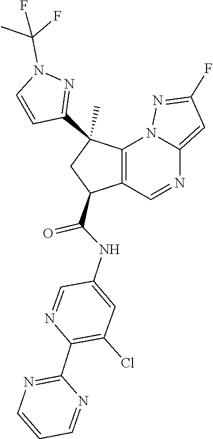 [SMILES: CC(F)(F)n1ccc([C@]2(C)C[C@H](C(=O)Nc3cnc(-c4ncccn4)c(Cl)c3)c3cnc4cc(F)nn4c32)n1] | |
| 171 (second eluting isomer)* 172 (first eluting isomer)* | 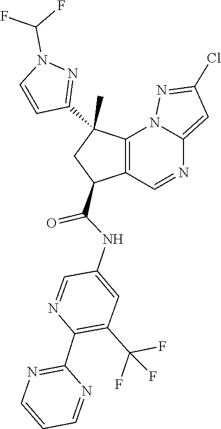 [SMILES: C[C@]1(c2ccn(C(F)F)n2)C[C@H](C(=O)Nc2cnc(-c3ncccn3)c(C(F)(F)F)c2)c2cnc3cc(Cl)nn3c21] | 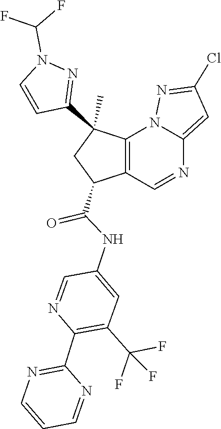 [SMILES: C[C@@]1(c2ccn(C(F)F)n2)C[C@@H](C(=O)Nc2cnc(-c3ncccn3)c(C(F)(F)F)c2)c2cnc3cc(Cl)nn3c21] |
| 173 (second eluting isomer)* 174 (first eluting isomer)* | 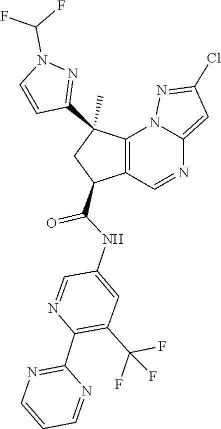 [SMILES: C[C@@]1(c2ccn(C(F)F)n2)C[C@H](C(=O)Nc2cnc(-c3ncccn3)c(C(F)(F)F)c2)c2cnc3cc(Cl)nn3c21] | 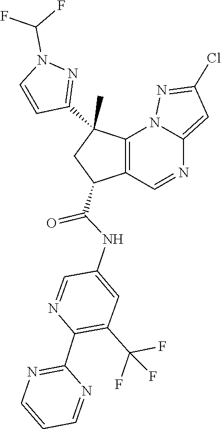 [SMILES: C[C@]1(c2ccn(C(F)F)n2)C[C@@H](C(=O)Nc2cnc(-c3ncccn3)c(C(F)(F)F)c2)c2cnc3cc(Cl)nn3c21] |
| 175 (second eluting isomer)* 176 (first eluting isomer)* | 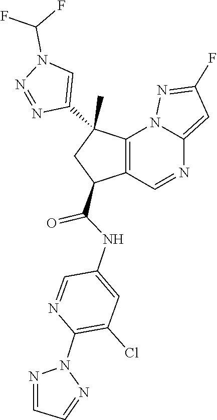 [SMILES: C[C@]1(c2cn(C(F)F)nn2)C[C@H](C(=O)Nc2cnc(-n3nccn3)c(Cl)c2)c2cnc3cc(F)nn3c21] | 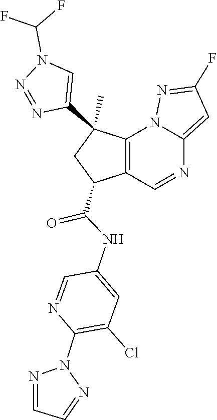 [SMILES: C[C@@]1(c2cn(C(F)F)nn2)C[C@@H](C(=O)Nc2cnc(-n3nccn3)c(Cl)c2)c2cnc3cc(F)nn3c21] |
| 177 (second eluting isomer)* 178 (first eluting isomer)* | 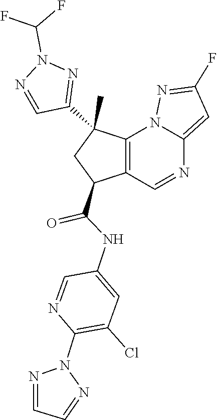 [SMILES: C[C@]1(c2cnn(C(F)F)n2)C[C@H](C(=O)Nc2cnc(-n3nccn3)c(Cl)c2)c2cnc3cc(F)nn3c21] | 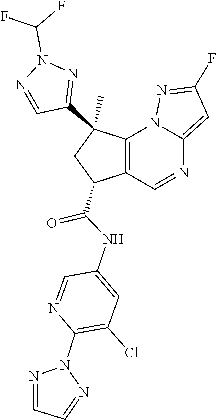 [SMILES: C[C@@]1(c2cnn(C(F)F)n2)C[C@@H](C(=O)Nc2cnc(-n3nccn3)c(Cl)c2)c2cnc3cc(F)nn3c21] |
| 179 (first eluting isomer)* 180 (second eluting isomer)* | 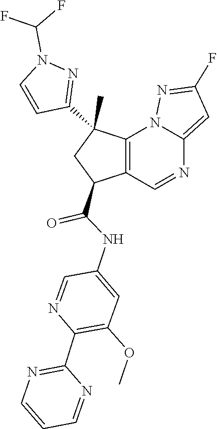 [SMILES: COc1cc(NC(=O)[C@H]2C[C@](C)(c3ccn(C(F)F)n3)c3c2cnc2cc(F)nn32)cnc1-c1ncccn1] | 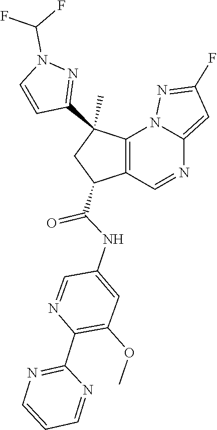 [SMILES: COc1cc(NC(=O)[C@@H]2C[C@@](C)(c3ccn(C(F)F)n3)c3c2cnc2cc(F)nn32)cnc1-c1ncccn1] |
| 181 (first eluting isomer)* 182 (second eluting isomer)* | 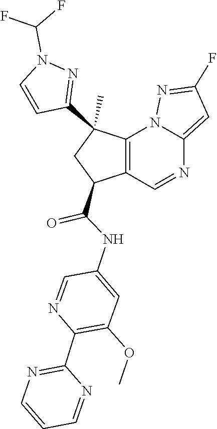 [SMILES: COc1cc(NC(=O)[C@H]2C[C@@](C)(c3ccn(C(F)F)n3)c3c2cnc2cc(F)nn32)cnc1-c1ncccn1] | 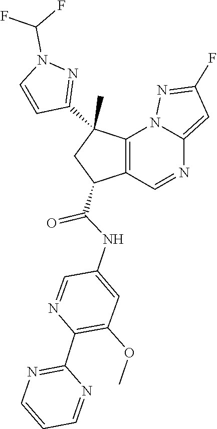 [SMILES: COc1cc(NC(=O)[C@@H]2C[C@](C)(c3ccn(C(F)F)n3)c3c2cnc2cc(F)nn32)cnc1-c1ncccn1] |
| 183 and 184 | 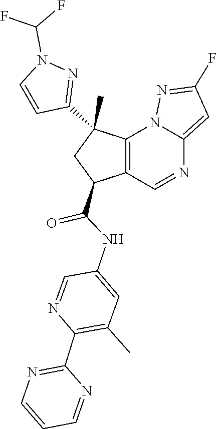 [SMILES: Cc1cc(NC(=O)[C@H]2C[C@](C)(c3ccn(C(F)F)n3)c3c2cnc2cc(F)nn32)cnc1-c1ncccn1] | 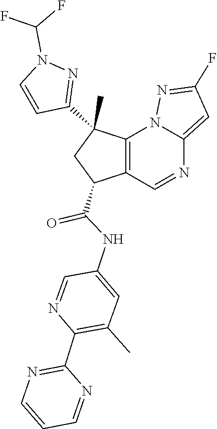 [SMILES: Cc1cc(NC(=O)[C@@H]2C[C@](C)(c3ccn(C(F)F)n3)c3c2cnc2cc(F)nn32)cnc1-c1ncccn1] |
| 185 (second eluting isomer)* 186 (first eluting isomer)* | 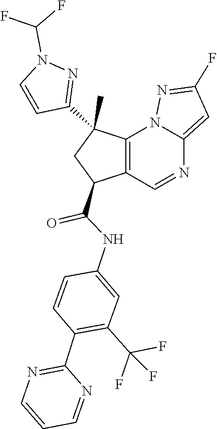 [SMILES: C[C@]1(c2ccn(C(F)F)n2)C[C@H](C(=O)Nc2ccc(-c3ncccn3)c(C(F)(F)F)c2)c2cnc3cc(F)nn3c21] | 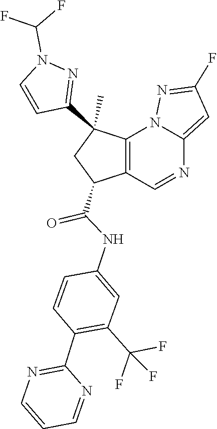 [SMILES: C[C@@]1(c2ccn(C(F)F)n2)C[C@@H](C(=O)Nc2ccc(-c3ncccn3)c(C(F)(F)F)c2)c2cnc3cc(F)nn3c21] |
| 187 (second eluting isomer)* 188 (first eluting isomer)* | 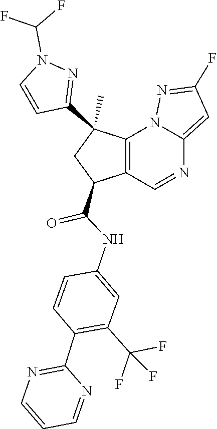 [SMILES: C[C@@]1(c2ccn(C(F)F)n2)C[C@H](C(=O)Nc2ccc(-c3ncccn3)c(C(F)(F)F)c2)c2cnc3cc(F)nn3c21] | 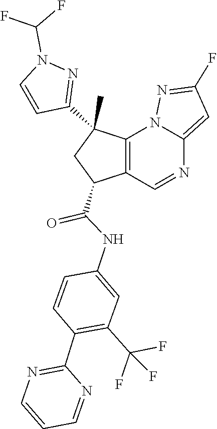 [SMILES: C[C@]1(c2ccn(C(F)F)n2)C[C@@H](C(=O)Nc2ccc(-c3ncccn3)c(C(F)(F)F)c2)c2cnc3cc(F)nn3c21] |
| 189 (second eluting isomer)* 190 (first eluting isomer)* | 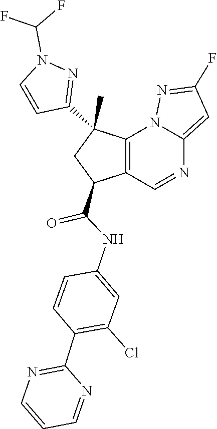 [SMILES: C[C@]1(c2ccn(C(F)F)n2)C[C@H](C(=O)Nc2ccc(-c3ncccn3)c(Cl)c2)c2cnc3cc(F)nn3c21] | 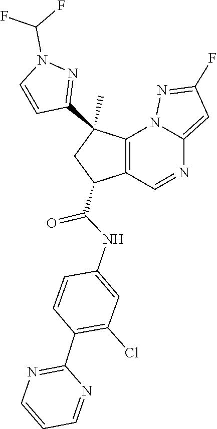 [SMILES: C[C@@]1(c2ccn(C(F)F)n2)C[C@@H](C(=O)Nc2ccc(-c3ncccn3)c(Cl)c2)c2cnc3cc(F)nn3c21] |
| 191 (first eluting isomer)* 192 (second eluting isomer)* | 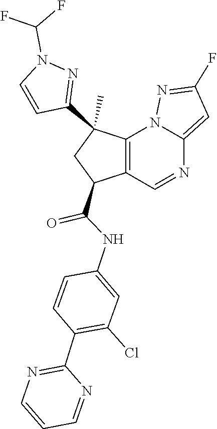 [SMILES: C[C@@]1(c2ccn(C(F)F)n2)C[C@H](C(=O)Nc2ccc(-c3ncccn3)c(Cl)c2)c2cnc3cc(F)nn3c21] | 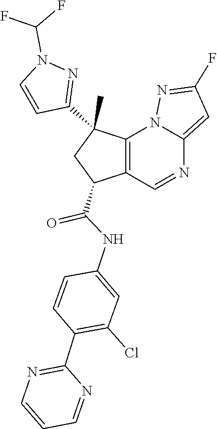 [SMILES: C[C@]1(c2ccn(C(F)F)n2)C[C@@H](C(=O)Nc2ccc(-c3ncccn3)c(Cl)c2)c2cnc3cc(F)nn3c21] |
| 193 and 194 | 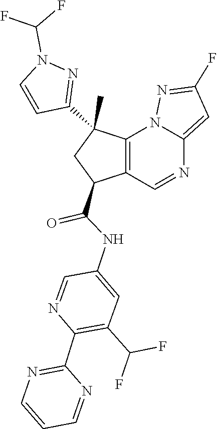 [SMILES: C[C@]1(c2ccn(C(F)F)n2)C[C@H](C(=O)Nc2cnc(-c3ncccn3)c(C(F)F)c2)c2cnc3cc(F)nn3c21] | 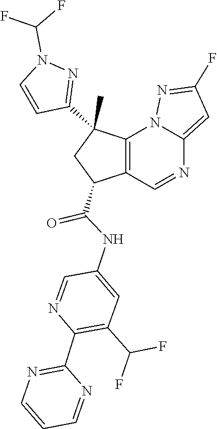 [SMILES: C[C@]1(c2ccn(C(F)F)n2)C[C@@H](C(=O)Nc2cnc(-c3ncccn3)c(C(F)F)c2)c2cnc3cc(F)nn3c21] |
| 195 and 196 | 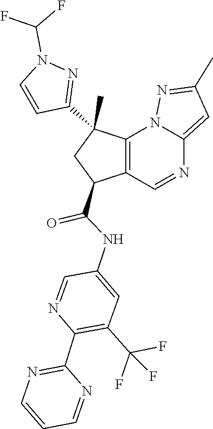 [SMILES: Cc1cc2ncc3c(n2n1)[C@@](C)(c1ccn(C(F)F)n1)C[C@@H]3C(=O)Nc1cnc(-c2ncccn2)c(C(F)(F)F)c1] | 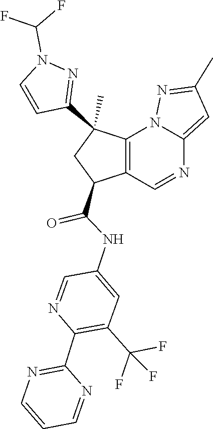 [SMILES: Cc1cc2ncc3c(n2n1)[C@](C)(c1ccn(C(F)F)n1)C[C@@H]3C(=O)Nc1cnc(-c2ncccn2)c(C(F)(F)F)c1] |

*See experimental procedures on details for separation of isomers. The absolute stereochemistry for each separated
isomer was not determined.

### Processes of Preparation

Provided herein is a process of preparing a compound of Formula (I) (e.g., any compound described herein), comprising:
reacting a compound of Formula (Ih)

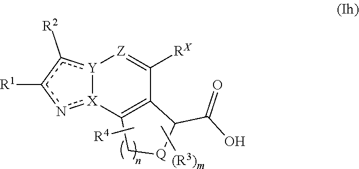

with R⁵—NH₂;
to form the compound of Formula (I).

In some embodiments, reacting the compound of Formula (Ih) with R⁵—NH₂ (e.g., 5-chloro-6-(triazolyl)pyridin-3-amine) is performed in the presence of POCl₃ and pyridine.

In some embodiments, reacting the compound of Formula (Ih) with R⁵—NH₂ is performed in the presence of N,N,N′,N′-tetramethylchloroformamidinium hexafluorophosphate (TCFH).

In some embodiments, reacting the compound of Formula (Ih) with R⁵—NH₂ is performed in the presence of N-methylimidazole (NMI).

In some embodiments, the compound of Formula (Ih) is prepared from a compound of Formula (Ih-N):

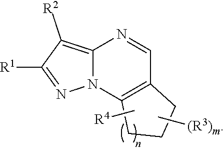

In some embodiments (when the compound of Formula (Ih) is prepared from a compound of Formula (Ih-N)), the process comprises reacting a compound of Formula (Ih-N-i)

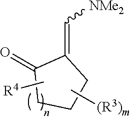

with a compound of Formula (Ih-N-ii)

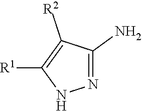

to form the compound of Formula (Ih-N).

In certain embodiments, reacting the compound of Formula (Ih-N-i) with the compound of Formula (Ih-N-ii) is performed in the presence of acid.

In certain of these embodiments, the acid is selected from the group consisting of hydrochloric acid and acetic acid.

Provided herein is a process of preparing a compound of Formula (I) (e.g., any compound described herein), comprising:
reacting a compound of Formula (Ij)

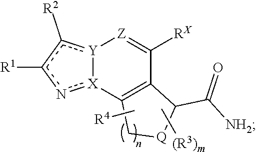

with R⁵-Hal, where Hal is selected from the group consisting of Cl, Br, I, and OSO₂CF₃;
to form the compound of Formula (I).

In some embodiments, reacting the compound of Formula (Ij) with R⁵-Hal is performed in the presence of a catalyst and a ligand.

In some embodiments (when reacting the compound of Formula (Ij) with R⁵-Hal is performed in the presence of a catalyst and a ligand), the catalyst is tris(dibenzylideneacetone)dipalladium(O).

In some embodiments (when reacting the compound of Formula (Ij) with R⁵-Hal is performed in the presence of a catalyst and a ligand), the ligand is 4,5-bis(diphenylphosphino)-9,9-dimethylxanthene.

In some embodiments, the compound of Formula (Ij) is prepared from a compound of Formula (Ih-N):

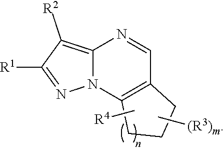

In some embodiments, the process comprises reacting a compound of Formula (Ih-N-i)

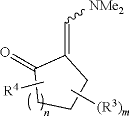

with a compound of Formula (Ih-N-ii)

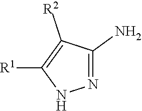

to form the compound of Formula (Ih-N).

In some embodiments, reacting the compound of Formula (Ih-N-i) with the compound of Formula (Ih-N-ii) is performed in the presence of acid.

In some embodiments (when reacting the compound of Formula (Ih-N-i) with the compound of Formula (Ih-N-ii) is performed in the presence of acid), the acid is selected from the group consisting of hydrochloric acid and acetic acid.

### Methods of Treatment

Some embodiments provide a method of treating an autoimmune disorder (e.g., a MALT1-associated autoimmune disorder) in a subject in need of such treatment, the method comprising administering to the subject an effective amount of a compound of Formula (I), or a pharmaceutically acceptable salt thereof, or a pharmaceutical composition thereof. In some embodiments, the autoimmune disorder is rheumatoid arthritis, multiple sclerosis, or systemic lupus erythematosus (SLE).

Some embodiments provide a method of treating an inflammatory disorder (e.g., a MALT1-associated inflammatory disorder) in a subject in need of such treatment, the method comprising administering to the subject an effective amount of a compound of Formula (I), or a pharmaceutically acceptable salt thereof, or a pharmaceutical composition thereof. In some embodiments, the inflammatory disorder is chronic graft versus host disease (cGVHD).

Some embodiments provide a method of treating cancer (e.g., a MALT1-associated cancer) in a subject in need of such treatment, the method comprising administering to the subject an effective amount of a compound of Formula (I), or a pharmaceutically acceptable salt thereof, or a pharmaceutical composition thereof. For example, provided herein are methods for treating a MALT1-associated cancer in a subject in need of such treatment, the method comprising a) detecting a dysregulation of a MALT1 gene, a MALT1 protease, or the expression or activity or level of any of the same in a sample from the subject; and b) administering an effective amount of a compound of Formula (I), or a pharmaceutically acceptable salt thereof. In some embodiments, the dysregulation of a MALT1 gene, a MALT1 protease, or the expression or activity or level of any of the same includes one or more fusion proteins.

In some embodiments of any of the methods or uses described herein, the cancer (e.g., MALT1-associated cancer) is a hematological cancer. In some embodiments of any of the methods or uses described herein, the cancer (e.g., MALT1-associated cancer) is a solid tumor. In some embodiments of any of the methods or uses described herein, the cancer (e.g., MALT1-associated cancer) is a lung cancer (e.g., small cell lung carcinoma or non-small cell lung carcinoma), thyroid cancer (e.g., papillary thyroid cancer, medullary thyroid cancer (e.g., sporadic medullary thyroid cancer or hereditary medullary thyroid cancer), differentiated thyroid cancer, recurrent thyroid cancer, or refractory differentiated thyroid cancer), thyroid adenoma, endocrine gland neoplasms, lung adenocarcinoma, bronchioles lung cell carcinoma, multiple endocrine neoplasia type 2A or 2B (MEN2A or MEN2B, respectively), pheochromocytoma, parathyroid hyperplasia, breast cancer, mammary cancer, mammary carcinoma, mammary neoplasm, colorectal cancer (e.g., metastatic colorectal cancer), papillary renal cell carcinoma, ganglioneuromatosis of the gastroenteric mucosa, inflammatory myofibroblastic tumor, or cervical cancer. In some embodiments of any of the methods or uses described herein, the cancer (e.g., MALT1-associated cancer) is selected from the group of: acute lymphoblastic leukemia (ALL), acute myeloid leukemia (AML), cancer in adolescents, adrenocortical carcinoma, anal cancer, appendix cancer, astrocytoma, atypical teratoid/rhabdoid tumor, basal cell carcinoma, bile duct cancer, bladder cancer, bone cancer, brain stem glioma, brain tumor, breast cancer, bronchial tumor, Burkitt lymphoma, carcinoid tumor, unknown primary carcinoma, cardiac tumors, cervical cancer, childhood cancers, chordoma, chronic lymphocytic leukemia (CLL), chronic myelogenous leukemia (CML), chronic myeloproliferative neoplasms, neoplasms by site, neoplasms, colon cancer, colorectal cancer, craniopharyngioma, cutaneous T-cell lymphoma, cutaneous angiosarcoma, bile duct cancer, ductal carcinoma in situ, embryonal tumors, endometrial cancer, ependymoma, esophageal cancer, esthesioneuroblastoma, Ewing sarcoma, extracranial germ cell tumor, extragonadal germ cell tumor, extrahepatic bile duct cancer, eye cancer, fallopian tube cancer, fibrous histiocytoma of bone, gallbladder cancer, gastric cancer, gastrointestinal carcinoid tumor, gastrointestinal stromal tumors (GIST), germ cell tumor, gestational trophoblastic disease, glioma, hairy cell tumor, hairy cell leukemia, head and neck cancer, thoracic neoplasms, head and neck neoplasms, CNS tumor, primary CNS tumor, heart cancer, hepatocellular cancer, histiocytosis, Hodgkin's lymphoma, hypopharyngeal cancer, intraocular melanoma, islet cell tumors, pancreatic neuroendocrine tumors, Kaposi sarcoma, kidney cancer, Langerhans cell histiocytosis, laryngeal cancer, leukemia, lip and oral cavity cancer, liver cancer, lung cancer, lymphoma, macroglobulinemia, malignant fibrous histiocytoma of bone, osteocarcinoma, melanoma, Merkel cell carcinoma, mesothelioma, metastatic squamous neck cancer, midline tract carcinoma, mouth cancer, multiple endocrine neoplasia syndromes, multiple myeloma, mycosis fungoides, myelodysplastic syndromes, myelodysplastic/myeloproliferative neoplasms, neoplasms by site, neoplasms, myelogenous leukemia, myeloid leukemia, multiple myeloma, myeloproliferative neoplasms, nasal cavity and paranasal sinus cancer, nasopharyngeal cancer, neuroblastoma, non-Hodgkin's lymphoma, non-small cell lung cancer, lung neoplasm, pulmonary cancer, pulmonary neoplasms, respiratory tract neoplasms, bronchogenic carcinoma, bronchial neoplasms, oral cancer, oral cavity cancer, lip cancer, oropharyngeal cancer, osteosarcoma, ovarian cancer, pancreatic cancer, papillomatosis, paraganglioma, paranasal sinus and nasal cavity cancer, parathyroid cancer, penile cancer, pharyngeal cancer, pheochromosytoma, pituitary cancer, plasma cell neoplasm, pleuropulmonary blastoma, pregnancy-associated breast cancer, primary central nervous system lymphoma, primary peritoneal cancer, prostate cancer, rectal cancer, colon cancer, colonic neoplasms, renal cell cancer, rhabdomyosarcoma, salivary gland cancer, sarcoma, Sezary syndrome, skin cancer, Spitz tumors, small cell lung cancer, small intestine cancer, soft tissue sarcoma, squamous cell carcinoma, squamous neck cancer, stomach cancer, T-cell lymphoma, testicular cancer, throat cancer, thymoma and thymic carcinoma, thyroid cancer, transitional cell cancer of the renal pelvis, unknown primary carcinoma, urethral cancer, uterine cancer, uterine sarcoma, vaginal cancer, vulvar cancer, and Wilms' tumor.

In some embodiments, the cancer is a hematological cancer, such as a leukemia or a lymphoma. In some embodiments, a hematological cancer (e.g., hematological cancers that are MALT1-associated cancers) is selected from the group consisting of leukemias, lymphomas (non-Hodgkin's lymphoma), Hodgkin's disease (also called Hodgkin's lymphoma), and myeloma, for instance, acute lymphocytic leukemia (ALL), acute myeloid leukemia (AML), acute promyelocytic leukemia (APL), chronic lymphocytic leukemia (CLL), chronic myeloid leukemia (CML), chronic myelomonocytic leukemia (CMML), chronic neutrophilic leukemia (CNL), acute undifferentiated leukemia (AUL), anaplastic large-cell lymphoma (ALCL), prolymphocytic leukemia (PML), juvenile myelomonocyctic leukemia (JMML), adult T-cell ALL, AML with trilineage myelodysplasia (AML/TMDS), mixed lineage leukemia (MLL), myelodysplastic syndromes (MDSs), myeloproliferative disorders (MPD), and multiple myeloma (MM).

Additional examples of hematological cancers include mycloproliferativc disorders (MPD) such as polycythemia vera (PV), essential thrombocytopenia (ET) and idiopathic primary myelofibrosis (IMF/IPF/PMF). In some embodiments, the hematological cancer (e.g., the hematological cancer that is a MALT1-associated cancer) is AML or CMML.

In some embodiments, the cancer is glioblastoma, chronic myelogenous leukemia, myeloid leukemia, or non-Hodgkin's lymphoma.

In some embodiments, cancer is non-Hodgkin's lymphoma. In other embodiments, the cancer is diffuse large B-cell lymphoma (DLBCL). In other embodiments the cancer is activated B-cell diffuse large B-cell lymphoma (ABC-DLBCL). In other embodiments the cancer is mantel cell lymphoma (MCL). In other embodiments, the cancer is marginal zone lymphoma (MZL). In other embodiments the cancer is chronic lymphatic leukemia (CLL). In other embodiments the cancer is mature B-cell neoplasms.

In some embodiments the cancer is T-cell acute lymphoblastic leukemia.

In some embodiments, the cancer (e.g., the MALT1-associated cancer) is a solid tumor. Examples of solid tumors (e.g., solid tumors that are MALT1-associated cancers) include, for example, lung cancer (e.g., lung adenocarcinoma, small-cell lung carcinoma), pancreatic cancer, pancreatic ductal carcinoma, breast cancer, colon cancer, colorectal cancer, prostate cancer, renal cell carcinoma, neuroblastoma, and melanoma. See, e.g., Jiang et al., Cancer Research 2011, 71, 2183-2192; see also, Pan et al., Mol Cancer Res 2016, 14, 93-102 and Penas et al., Blood 2010, 115, 2214-2219.

In some embodiments, the subject is a human.

Compounds of Formula (I) and pharmaceutically acceptable salts thereof are also useful for treating a MALT1-associated cancer. Compounds of Formula (I) and pharmaceutically acceptable salts thereof are also useful for treating a MALT1-associated autoimmune disorder.

Compounds of Formula (I) and pharmaceutically acceptable salts thereof are also useful for treating a MALT1-associated inflammatory disease.

Accordingly, also provided herein is a method for treating a subject diagnosed with or identified as having a MALT1-associated cancer, e.g., any of the exemplary MALT1-associated cancers disclosed herein, comprising administering to the subject an effective amount of a compound of Formula (I), or a pharmaceutically acceptable salt thereof, or a pharmaceutical composition thereof as defined herein.

In some embodiments of any of the methods provided herein, a compound of Formula (I) is selected from Examples 1-196.

Dysregulation of a MALT1 protease, a MALT1 gene, or the expression or activity or level of any (e.g., one or more) of the same can contribute to tumorigenesis. For example, a fusion protein can have increased protease activity as compared to a wild-type MALT1 protein, increased expression (e.g., increased levels) of a wild-type MALT1 protease in a mammalian cell can occur due to aberrant cell signaling and/or dysregulated autocrine/paracrine signaling (e.g., as compared to a control non-cancerous cell), MALT1 mRNA splice variants may also result in dysregulation of MALT1.

In some aspects, provided herein is a method for treating cancer in a subject in need thereof, including administering to the subject an effective amount of a compound of Formula (I), or a pharmaceutically acceptable salt thereof. Also provided herein is a method for treating a CBM complex pathway-associated cancer (such as any of those disclosed herein) in a subject in need thereof, including administering to the subject an effective amount of a compound of Formula (I), or a pharmaceutically acceptable salt thereof. Also provided is a method for treating a cancer in a subject in need thereof, including (a) identifying the cancer as being a CBM complex pathway-associated cancer; and (b) administering to the subject an effective amount of a compound of Formula (I), or a pharmaceutically acceptable salt thereof.

Identifying the cancer identifying the cancer in the subject as a CBM complex pathway-associated cancer can be performed by any appropriate method. In some embodiments, the step of identifying the cancer in the subject as a CBM complex pathway-associated cancer includes performing an assay to detect dysregulation in a CBM complex pathway-associated gene, a CBM complex pathway-associated protease protein, or expression or activity or level of any of the same in a sample from the subject. In some embodiments, the method further includes obtaining a sample from the subject (e.g., a biopsy sample). An assay can be any appropriate assay. In some embodiments, the assay is selected from the group consisting of sequencing (e.g., pyrosequencing or next generation sequencing), immunohistochemistry, enzyme-linked immunosorbent assay, and fluorescence in situ hybridization (FISH).

Also provided herein is a method for treating a cancer in a subject in need thereof, including administering to the subject an effective amount of a compound of Formula (I), or a pharmaceutically acceptable salt thereof to a subject identified as having a CBM complex pathway-associated cancer.

Also provided herein is a method of treating a MALT1-associated cancer in a subject, including administering to a subject identified or diagnosed as having a MALT1-associated cancer an effective amount of a compound of Formula (I), or a pharmaceutically acceptable salt thereof.

Provided herein is also a method for treating cancer in a subject in need thereof, including: (a) determining that the cancer is associated with a dysregulation of a MALT1 gene, a MALT1 protease, or expression or activity or level of any of the same; and (b) administering to the subject an effective amount of a compound of Formula (I), or a pharmaceutically acceptable salt thereof.

Determining that the cancer is associated with a dysregulation of a MALT1 gene, a MALT1 protease, or expression or activity or level of any of the same can be performed using any appropriate method. In some embodiments, the step of determining that the cancer in the subject is a MALT1-associated cancer includes performing an assay to detect dysregulation in a MALT1 gene, a MALT1 protease protein, or expression or activity or level of any of the same in a sample from the subject. In some embodiments, the method further includes obtaining a sample from the subject (e.g., a biopsy sample). An assay can be any appropriate assay. In some embodiments, the assay is selected from the group consisting of sequencing (e.g., pyrosequencing or next generation sequencing), immunohistochemistry, enzyme-linked immunosorbent assay, and fluorescence in situ hybridization (FISH).

As described herein, a CBM complex pathway-associated cancer can be any appropriate CBM complex pathway-associated cancer (such as any of those described herein). In some embodiments, a CBM complex pathway-associated cancer is selected from the group consisting of a CBM complex pathway cell surface receptor-associated cancer, a cancer associated with a signal transducer between a cell surface receptor and a CBM complex, a component of a CBM complex-associated cancer, a MALT1 protease substrate-associated cancer, a cancer associated with a component of the NF-κB pathway downstream of a CBM complex, a cancer associated with a component of the JNK pathway downstream of a CBM complex, and a combination thereof. In some embodiments, the CBM complex pathway cell surface receptor-associated cancer is selected from the group consisting of a CD28-associated cancer, a BCR-associated cancer, a HER1-associated cancer, a HER2-associated cancer, and combinations thereof. In some embodiments, the cancer associated with a signal transducer between a cell surface receptor and a CBM complex is a protein kinase C beta (PKCβ)-associated cancer, a protein kinase C theta (PCKθ)-associated cancer, or a combination thereof. In some embodiments, the component of a CBM complex-associated cancer is selected from the group consisting of a MALT1-associated cancer, a CARD1-associated cancer, a CARD14-associated cancer, a CARD10-associated cancer, a CARD9-associated cancer, a BCL10-associated cancer, and combinations thereof. In some embodiments, the component of a CBM complex-associated cancer is selected from the group consisting of a MALT1-associated cancer, a CARD11-associated cancer, a BCL10-associated cancer, and combinations thereof. See, e.g., Tables B1, B2, and B3 for exemplary dysregulations in MALT1, CARD11, and BCL10. In some embodiments, the MALT1 protease substrate-associated cancer is selected from the group consisting of a BCL10-associated cancer, an A20-associated cancer, a CYLD-associated cancer, a RelB-associated cancer, a Regnase 1-associated cancer, a roquin-1-associated cancer, a HOIL1-associated cancer, a NIK associated cancer, a LIMA1α-associated cancer, and a combination thereof. In some embodiments, the MALT1 protease substrate-associated cancer is selected from the group consisting of a BCL10-associated cancer, an A20-associated cancer, a CYLD-associated cancer, and combinations thereof. See, e.g., Tables B3 and B4 for exemplary dysregulations in BCL10 and A20. In some embodiments, the cancer associated with a component of the NF-κB pathway downstream of a CBM complex is selected from the group consisting of a TAK1-associated cancer, a TRAF6-associated cancer, a TAB1-associated cancer, a TAB2-associated cancer, a TAB3-associated cancer, a MKK7-associated cancer, an IKKα-associated cancer, an IKKβ-associated cancer, an IKKγ-associated cancer, an IκBα-associated cancer, a p50-associated cancer, a p65 (RelA)-associated cancer, a c-Rel-associated cancer, and combinations thereof. In some embodiments, the cancer associated with a component of the NF-κB pathway downstream of a CBM complex is an IKKγ-associated cancer.

In some embodiments, the cancer associated with a component of the JNK pathway downstream of a CBM complex is selected from the group consisting of a JNK1-associated cancer, a JNK2-associated cancer, a JNK3-associated cancer, a MYD88 transcription factor-associated cancer, an AP-1 transcription factor-associated cancer, and combinations thereof.

In some embodiments, the CBM complex pathway-associated cancer is a MALT1-associated cancer. A MALT1-associated cancer can have any appropriate dysregulation, such as any of those described herein. In some embodiments, the MALT1-associated cancer comprises an IAP2-MALT1 fusion. In some embodiments, the MALT1-associated cancer comprises an IGH-MALT1 fusion.

Also provided herein are methods of treating CBM complex pathway-associated diseases or disorders, autoimmune disorders, and inflammatory disorders. Accordingly, provided herein is a method for treating an autoimmune disorder in a subject in need thereof, including administering to the subject an effective amount of a compound of Formula (I), or a pharmaceutically acceptable salt thereof. Also provided herein is a method of treating a MALT1-associated autoimmune disorder in a subject, including administering to a subject identified or diagnosed as having a MALT1-associated autoimmune disorder an effective amount of a compound of Formula (I), or a pharmaceutically acceptable salt thereof. In some cases, provided herein is a method for treating an autoimmune disorder in a subject in need thereof, including: (a) determining that the autoimmune disorder is associated with a dysregulation of a MALT1 gene, a MALT1 protease, or expression or activity or level of any of the same; and (b) administering to the subject an effective amount of a compound of Formula (I), or a pharmaceutically acceptable salt thereof. Provided also herein is a method of treating a MALT1-associated autoimmune disorder in a subject, including administering an effective amount of a compound of Formula (I), or a pharmaceutically acceptable salt thereof to a subject determined to have a MALT1-associated autoimmune disorder. In addition, provided herein is a method for treating an inflammatory disorder in a subject in need thereof, including administering to the subject an effective amount of a compound of Formula (I), or a pharmaceutically acceptable salt thereof. In some cases, provided herein is a method of treating a MALT1-associated inflammatory disorder in a subject, including administering to a subject identified or diagnosed as having a MALT1-associated inflammatory disorder an effective amount of a compound of Formula (I), or a pharmaceutically acceptable salt thereof. Also provided herein is a method for treating an inflammatory disorder in a subject in need thereof, including: (a) determining that the inflammatory disorder is associated with a dysregulation of a MALT1 gene, a MALT1 protease, or expression or activity or level of any of the same; and (b) administering to the subject an effective amount of a compound of Formula (I), or a pharmaceutically acceptable salt thereof. Provided also herein is a method of treating a MALT1-associated inflammatory disorder in a subject, including administering an effective amount of a compound of Formula (I), or a pharmaceutically acceptable salt thereof to a subject determined to have a MALT1-associated inflammatory disorder

Additionally provided herein is a method for treating a CBM complex pathway-associated disease or disorder in a subject in need thereof, including administering to the subject an effective amount of a compound of Formula (I), or a pharmaceutically acceptable salt thereof. Also provided is a method for treating a disease or disorder in a subject in need thereof, including: (a) identifying the cancer as being a CBM complex pathway-associated disease or disorder; and (b) administering to the subject an effective amount of a compound of Formula (I), or a pharmaceutically acceptable salt thereof. In addition, provided herein is a method for treating a disease or disorder in a subject in need thereof, including: administering to the subject an effective amount of a compound of Formula (I), or a pharmaceutically acceptable salt thereof to a subject identified as having a CBM complex pathway-associated disease or disorder.

A CBM complex pathway-associated disease or disorder can be any appropriate CBM complex pathway-associated disease or disorder, such as any of those described herein. In some embodiments, the CBM complex pathway-associated disease or disorder is an autoimmune disease. In some embodiments, the CBM complex pathway-associated disease or disorder is an inflammatory disease. In some embodiments, the CBM complex pathway-associated cancer is selected from the group consisting of a CBM complex pathway cell surface receptor-associated cancer, a disease or disorder associated with a signal transducer between a cell surface receptor and a CBM complex, a component of a CBM complex-associated cancer, a MALT1 protease substrate-associated cancer, a disease or disorder associated with a component of the NF-κB pathway downstream of a CBM complex, a disease or disorder associated with a component of the JNK pathway downstream of a CBM complex, and a combination thereof. In some embodiments, the CBM complex pathway-associated disease or disorder is a MALT1-associated disease or disorder.

In some cases, compounds of Formula (I), or a pharmaceutically acceptable salt thereof can be useful for inhibiting the processes of cells, such as inhibiting the proliferation of cells.

Accordingly, provided herein is a method for inhibiting mammalian cell proliferation, including contacting the mammalian cell with a compound of Formula (I), or a pharmaceutically acceptable salt thereof. Also provided herein is a method for inhibiting CBM complex pathway activity in a mammalian cell, including contacting the mammalian cell with a compound of Formula (I), or a pharmaceutically acceptable salt thereof. Provided also herein is a method for inhibiting MALT1 protease activity in a mammalian cell, including contacting the mammalian cell with a compound of Formula (I), or a pharmaceutically acceptable salt thereof. In some embodiments, the contacting occurs in vivo. In some embodiments, the contacting occurs in vitro. A mammalian cell can be any appropriate cell. In some embodiments, the mammalian cell is a mammalian immune cell. In some embodiments, the mammalian cell is a mammalian cancer cell. In some embodiments, the mammalian cancer cell is a mammalian CBM complex pathway-associated cancer cell. In some embodiments, the mammalian cancer cell is a mammalian MALT1-associated cancer cell. In some embodiments, the mammalian cell has dysregulation of a MALT1 gene, a MALT1 protease protein, or expression or activity or level of any of the same. In some embodiments, the dysregulation of a MALT1 gene, a MALT1 protease protein, or expression or activity or level of any of the same is an IAP2-MALT1 fusion, an IGH-MALT1 fusion, or a combination thereof.

Compounds of Formula (I), or a pharmaceutically acceptable salt thereof can also be useful in the manufacture of medicaments. Accordingly, provided herein is a use of a compound of Formula (I), or a pharmaceutically acceptable salt thereof in the manufacture of a medicament for the treatment of a CBM complex pathway-associated disease or disorder. A CBM complex pathway-associated disease or disorder can be any appropriate CBM complex pathway-associated disease or disorder, such as those described herein. In some embodiments, the CBM complex pathway-associated disease or disorder is selected from the group consisting of a CBM complex pathway cell surface receptor-associated cancer, a disease or disorder associated with a signal transducer between a cell surface receptor and a CBM complex, a component of a CBM complex-associated cancer, a MALT1 protease substrate-associated cancer, a disease or disorder associated with a component of the NF-κB pathway downstream of a CBM complex, a disease or disorder associated with a component of the JNK pathway downstream of a CBM complex, and a combination thereof. In some embodiments, the CBM complex pathway-associated disease or disorder is a CBM complex pathway-associated autoimmune disorder. In some embodiments, the CBM complex pathway-associated disease or disorder is a CBM complex pathway-associated inflammatory disorder. In some embodiments, the CBM complex pathway-associated disease or disorder is a CBM complex pathway-associated cancer. In some embodiments, the CBM complex pathway-associated disease or disorder is a MALT1-associated disease or disorder. In some embodiments, the MALT1-associated disease or disorder comprises a dysregulation of a MALT1 gene, a MALT1 protease protein, or expression or activity or level of any of the same. In some embodiments, the dysregulation of a MALT1 gene, a MALT1 protease protein, or expression or activity or level of any of the same is an IAP2-MALT1 fusion, an IGH-MALT1 fusion, or a combination thereof.

In some embodiments, the compounds provided herein exhibit brain and/or central nervous system (CNS) penetrance. Such compounds are capable of crossing the blood brain barrier and inhibiting a MALT1 protease in the brain and/or other CNS structures. In some embodiments, the compounds provided herein are capable of crossing the blood brain barrier in an effective amount. For example, treatment of a subject with cancer (e.g., a MALT1-associated cancer such as a MALT1-associated brain or CNS cancer) can include administration (e.g., oral administration) of the compound to the subject. In some such embodiments, the compounds provided herein are useful for treating a primary brain tumor or metastatic brain tumor. For example, the compounds can be used in the treatment of one or more of gliomas such as glioblastoma (also known as glioblastoma multiforme), astrocytomas, oligodendrogliomas, ependymomas, and mixed gliomas, meningiomas, medulloblastomas, gangliogliomas, schwannomas (neurilemmomas), and craniopharyngiomas (see, for example, the tumors listed in Louis, D. N. et al. Acta Neuropathol 131(6), 803-820 (June 2016)). In some embodiments, the brain tumor is a primary brain tumor. In some embodiments, the subject has previously been treated with another anticancer agent, e.g., another protease inhibitor (e.g., a compound that is not a compound of Formula (I)). In some embodiments, the brain tumor is a metastatic brain tumor. In some embodiments, the subject has previously been treated with another anticancer agent, e.g., another protease inhibitor (e.g., a compound that is not a compound of Formula (I)).

In some embodiments of any of the methods or uses described herein, an assay used to determine whether the subject has a dysregulation of a gene (e.g., a MALT1 gene), or a protein (e.g., a MALT1 protein), or expression or activity or level of any of the same, using a sample from a subject can include, for example, next generation sequencing, immunohistochemistry, fluorescence microscopy, break apart FISH analysis, Southern blotting, Western blotting, FACS analysis, Northern blotting, and PCR-based amplification (e.g., RT-PCR and quantitative real-time RT-PCR). As is well-known in the art, the assays are typically performed, e.g., with at least one labelled nucleic acid probe or at least one labelled antibody or antigen-binding fragment thereof. Assays can utilize other detection methods known in the art for detecting dysregulation of a gene (e.g., a MALT1 gene), a protein (e.g., a MALT1 protein), or expression or activity or levels of any of the same. In some embodiments, the sample is a biological sample or a biopsy sample (e.g., a paraffin-embedded biopsy sample) from the subject. In some embodiments, the subject is a subject suspected of having a MALT1-associated cancer, a subject having one or more symptoms of a MALT1-associated cancer, and/or a subject that has an increased risk of developing a MALT1-associated cancer).

In some embodiments, dysregulation of a gene (e.g., a MALT1 gene), a MALT1 protein (e.g., a MALT1 protein), or the expression or activity or level of any of the same can be identified using a liquid biopsy (variously referred to as a fluid biopsy or fluid phase biopsy). Liquid biopsy methods can be used to detect total tumor burden and/or the dysregulation of a gene (e.g., a MALT1 protein), a MALT1 protein (e.g., a MALT1 protein), or the expression or activity or level of any of the same. Liquid biopsies can be performed on biological samples obtained relatively easily from a subject (e.g., via a simple blood draw) and are generally less invasive than traditional methods used to detect tumor burden and/or dysregulation of a gene (e.g., a MALT1 gene), a protein (e.g., a MALT1 protein), or the expression or activity or level of any of the same. In some embodiments, liquid biopsies can be used to detect the presence of dysregulation of a gene (e.g., a MALT1 gene), a protein (e.g., a MALT1 protein), or the expression or activity or level of any of the same at an earlier stage than traditional methods. In some embodiments, the biological sample to be used in a liquid biopsy can include, blood, plasma, urine, cerebrospinal fluid, saliva, sputum, broncho-alveolar lavage, bile, lymphatic fluid, cyst fluid, stool, ascites, and combinations thereof. In some embodiments, a liquid biopsy can be used to detect circulating tumor cells (CTCs). In some embodiments, a liquid biopsy can be used to detect cell-free DNA. In some embodiments, cell-free DNA detected using a liquid biopsy is circulating tumor DNA (ctDNA) that is derived from tumor cells. Analysis of ctDNA (e.g., using sensitive detection techniques such as, without limitation, next-generation sequencing (NGS), traditional PCR, digital PCR, or microarray analysis) can be used to identify dysregulation of a gene (e.g., a MALT1 gene), a protein (e.g., a MALT1 protein), or the expression or activity or level of any of the same.

In some embodiments, ctDNA derived from a single gene can be detected using a liquid biopsy. In some embodiments, ctDNA derived from a plurality of genes (e.g., 2, 3, 4, 5, 6, 7, 8, 9, 10, 15, 20, 25, 30, 35, 40, 45, 50, 55, 60, 65, 70, 75, 80, 85, 90, 95, 100 or more, or any number of genes in between these numbers) can be detected using a liquid biopsy. In some embodiments, ctDNA derived from a plurality of genes can be detected using any of a variety of commercially-available testing panels (e.g., commercially available testing panels designed to detect dysregulation of a gene (e.g., a MALT1 gene), a protein (e.g., a MALT1 protein), or the expression or activity or level of any of the same). Liquid biopsies can be used to detect dysregulation of a gene (e.g., a MALT1 gene), a protein (e.g., a MALT1 protein), or the expression or activity or level of any of the same including, without limitation, point mutations or single nucleotide variants (SNVs), copy number variants (CNVs), genetic fusions (e.g., translocations or rearrangements), insertions, deletions, or any combination thereof. In some embodiments, a liquid biopsy can be used to detect a germline mutation. In some embodiments, a liquid biopsy can be used to detect a somatic mutation. In some embodiments, a liquid biopsy can be used to detect a primary genetic mutation (e.g., a primary mutation or a primary fusion that is associated with initial development of a disease, e.g., cancer). In some embodiments, a dysregulation of a gene (e.g., a MALT1 gene), a protein (e.g., a MALT1 protein), or the expression or activity or level of any of the same identified using a liquid biopsy is also present in a cancer cell that is present in the subject (e.g., in a tumor). In some embodiments, any of the types of dysregulation of a gene (e.g., a MALT1 gene), a protein (e.g., a MALT1 protein), or the expression or activity or level of any of the same described herein can be detected using a liquid biopsy. In some embodiments, a genetic mutation identified via a liquid biopsy can be used to identify the subject as a candidate for a particular treatment. For example, detection of dysregulation of a gene (e.g., a MALT1 gene), a protein (e.g., a MALT1 protein), or the expression or activity or level of any of the same in the subject can indicate that the subject will be responsive to a treatment that includes administration of a compound of Formula (T), or a pharmaceutically acceptable salt thereof.

Liquid biopsies can be performed at multiple times during a course of diagnosis, a course of monitoring, and/or a course of treatment to determine one or more clinically relevant parameters including, without limitation, progression of the disease and/or efficacy of a treatment. For example, a first liquid biopsy can be performed at a first time point and a second liquid biopsy can be performed at a second time point during a course of diagnosis, a course of monitoring, and/or a course of treatment. In some embodiments, the first time point can be a time point prior to diagnosing a subject with a disease (e.g., when the subject is healthy), and the second time point can be a time point after subject has developed the disease (e.g., the second time point can be used to diagnose the subject with the disease). In some embodiments, the first time point can be a time point prior to diagnosing a subject with a disease (e.g., when the subject is healthy), after which the subject is monitored, and the second time point can be a time point after monitoring the subject.

In some embodiments, the first time point can be a time point after diagnosing a subject with a disease, after which a treatment is administered to the subject, and the second time point can be a time point after the treatment is administered; in such cases, the second time point can be used to assess the efficacy of the treatment (e.g., if the genetic mutation(s) detected at the first time point are reduced in abundance or are undetectable). In some embodiments, a treatment to be administered to a subject can include a compound of Formula (I), or a pharmaceutically acceptable salt thereof.

In some embodiments, the efficacy of a compound of Formula (I), or a pharmaceutically acceptable salt thereof, can be determined by assessing the allele frequency of a dysregulation of a gene (e.g., a MALT1 gene) in cfDNA obtained from a subject at different time points, e.g., cfDNA obtained from the subject at a first time point and cfDNA obtained from the subject at a second time point, where at least one dose of a compound of Formula (I), or a pharmaceutically acceptable salt thereof, is administered to the subject between the first and second time points.

Some embodiments of these methods can further include administering to the subject at least one dose of the compound of Formula (I), or a pharmaceutically acceptable salt thereof, between the first and second time points. For example, a reduction (e.g., a 1% to about a 99% reduction, a 1% to about a 95% reduction, a 1% to about a 90% reduction, a 1% to about a 85% reduction, a 1% to about a 80% reduction, a 1% to about a 75% reduction, a 1% reduction to about a 70% reduction, a 1% reduction to about a 65% reduction, a 1% reduction to about a 60% reduction, a 1% reduction to about a 55% reduction, a 1% reduction to about a 50% reduction, a 1% reduction to about a 45% reduction, a 1% reduction to about a 40% reduction, a 1% reduction to about a 35% reduction, a 1% reduction to about a 30% reduction, a 1% reduction to about a 25% reduction, a 1% reduction to about a 20% reduction, a 1% reduction to about a 15% reduction, a 1% reduction to about a 10% reduction, a 1% to about a 5% reduction, about a 5% to about a 99% reduction, about a 10% to about a 99% reduction, about a 15% to about a 99% reduction, about a 20% to about a 99% reduction, about a 25% to about a 99% reduction, about a 30% to about a 99% reduction, about a 35% to about a 99% reduction, about a 40% to about a 99% reduction, about a 45% to about a 99% reduction, about a 50% to about a 99% reduction, about a 55% to about a 99% reduction, about a 60% to about a 99% reduction, about a 65% to about a 99% reduction, about a 70% to about a 99% reduction, about a 75% to about a 95% reduction, about a 80% to about a 99% reduction, about a 90% reduction to about a 99% reduction, about a 95% to about a 99% reduction, about a 5% to about a 10% reduction, about a 5% to about a 25% reduction, about a 10% to about a 30% reduction, about a 20% to about a 40% reduction, about a 25% to about a 50% reduction, about a 35% to about a 55% reduction, about a 40% to about a 60% reduction, about a 50% reduction to about a 75% reduction, about a 60% reduction to about 80% reduction, or about a 65% to about a 85% reduction) in the allele frequency (AF) of the dysregulation of a gene (e.g., MALT1 gene) in the cfDNA obtained from the subject at the second time point as compared to the allele frequency (AF) of the dysregulation of a gene (e.g., MALT1 gene) in the cfDNA obtained from the subject at the first time point indicates that the compound of Formula (I), or a pharmaceutically acceptable salt thereof, was effective in the subject. In some embodiments, the AF is reduced such that the level is below the detection limit of the instrument. Alternatively, an increase in the allele frequency (AF) of the dysregulation of a gene (e.g., MALT1 gene) in the cfDNA obtained from the subject at the second time point as compared to the allele frequency (AF) of the dysregulation of a gene (e.g., MALT1 gene) in the cfDNA obtained from the subject at the first time point indicates that the compound of Formula (I), or a pharmaceutically acceptable salt thereof, was not effective in the subject. Some embodiments of these methods can further include, administering additional doses of a compound of Formula (I), or a pharmaceutically acceptable salt thereof, to a subject in which a compound of Formula (I), or a pharmaceutically acceptable salt thereof, was determined to be effective. Some embodiments of these methods can further include, administering a different treatment (e.g., a treatment that does not include the administration of a compound of Formula (I), or a pharmaceutically acceptable salt thereof, as a monotherapy) to a subject in which a compound of Formula (I), or a pharmaceutically acceptable salt thereof, was determined not to be effective.

In some examples of these methods, the time difference between the first and second time points can be about 1 day to about 1 year, about 1 day to about 11 months, about 1 day to about 10 months, about 1 day to about 9 months, about 1 day to about 8 months, about 1 day to about 7 months, about 1 day to about 6 months, about 1 day to about 5 months, about 1 day to about 4 months, about 1 day to about 3 months, about 1 day to about 10 weeks, about 1 day to about 2 months, about 1 day to about 6 weeks, about 1 day to about 1 month, about 1 day to about 25 days, about 1 day to about 20 days, about 1 day to about 15 days, about 1 day to about 10 days, about 1 day to about 5 days, about 2 days to about 1 year, about 5 days to about 1 year, about 10 days to about 1 year, about 15 days to about 1 year, about 20 days to about 1 year, about 25 days to about 1 year, about 1 month to about 1 year, about 6 weeks to about 1 year, about 2 months to about 1 year, about 3 months to about 1 year, about 4 months to about 1 year, about 5 months to about 1 year, about 6 months to about 1 year, about 7 months to about 1 year, about 8 months to about 1 year, about 9 months to about 1 year, about 10 months to about 1 year, about 11 months to about 1 year, about 1 day to about 7 days, about 1 day to about 14 days, about 5 days to about 10 days, about 5 day to about 20 days, about 10 days to about 20 days, about 15 days to about 1 month, about 15 days to about 2 months, about 1 week to about 1 month, about 2 weeks to about 1 month, about 1 month to about 3 months, about 3 months to about 6 months, about 4 months to about 6 months, about 5 months to about 8 months, or about 7 months to about 9 months. In some embodiments of these methods, the subject can be previously identified as having a cancer having a dysregulated gene (e.g., any of the examples of a dysregulated gene described herein) (e.g., a MALT1 gene). In some embodiments of these methods, a subject can have been previously diagnosed as having any of the types of cancer described herein. In some embodiments of these methods, the subject can have one or more metastases (e.g., one or more brain metastases).

In some of the above embodiments, the cfDNA comprises ctDNA such as MALT1-associated ctDNA. For example, the cfDNA is ctDNA such as MALT1-associated ctDNA. In some embodiments, at least some portion of cfDNA is determined to be MALT1-associated ctDNA, for example, a sequenced and/or quantified amount of the total cfDNA is determined to have a MALT1 fusion and/or overexpression of MALT1.

Although the genetic basis of tumorigenesis may vary between different cancer types, the cellular and molecular mechanisms required for metastasis appear to be similar for all solid tumor types. During a metastatic cascade, the cancer cells lose growth inhibitory responses, undergo alterations in adhesiveness and produce enzymes that can degrade extracellular matrix components. This leads to detachment of tumor cells from the original tumor, infiltration into the circulation through newly formed vasculature, migration and extravasation of the tumor cells at favorable distant sites where they may form colonies.

Accordingly, also provided herein are methods for inhibiting, preventing, aiding in the prevention, or decreasing the symptoms of metastasis of a cancer in a subject in need thereof, the method comprising administering to the subject an effective amount of a compound of Formula (I), or a pharmaceutically acceptable salt thereof or a pharmaceutical composition thereof. Such methods can be used in the treatment of one or more of the cancers described herein. See, e.g., US Publication No. 2013/0029925; International Publication No. WO 2014/083567; and U.S. Pat. No. 8,568,998. See also, e.g., Hezam K et al., Rev Neurosci 2018 Jan 26; 29:93-98; Gao L, et al., Pancreas 2015 Jan; 44:134-143; Ding K et al., J Biol Chem 2014 Jun 6; 289:16057-71; and Amit M et al., Oncogene 2017 Jun 8; 36:3232-3239. In some embodiments, the cancer is a MALT1-associated cancer. In some embodiments, the compound of Formula (I), or a pharmaceutically acceptable salt thereof is used in combination with an additional therapy or another therapeutic agent, as described herein. For example, a first or second MALT1 protease inhibitor.

The term “metastasis” is an art known term and means the formation of an additional tumor (e.g., a solid tumor) at a site distant from a primary tumor in a subject, where the additional tumor includes the same or similar cancer cells as the primary tumor.

Also provided are methods of decreasing the risk of developing a metastasis or an additional metastasis in a subject having a MALT1-associated cancer that include: selecting, identifying, or diagnosing a subject as having a MALT1-associated cancer, and administering an effective amount of a compound of Formula (I), or a pharmaceutically acceptable salt thereof to the subject selected, identified, or diagnosed as having a MALT1-associated cancer. Also provided are methods of decreasing the risk of developing a metastasis or an additional metastasis in a subject having a MALT1-associated cancer that includes administering an effective amount of a compound of Formula (I), or a pharmaceutically acceptable salt thereof to a subject having a MALT1-associated cancer. The decrease in the risk of developing a metastasis or an additional metastasis in a subject having a MALT1-associated cancer can be compared to the risk of developing a metastasis or an additional metastasis in the subject prior to treatment, or as compared to a subject or a population of subjects having a similar or the same MALT1-associated cancer that has received no treatment or a different treatment.

The phrase “risk of developing a metastasis” means the risk that a subject having a primary tumor will develop an additional tumor (e.g., a solid tumor) at a site distant from a primary tumor in a subject over a set period of time, where the additional tumor includes the same or similar cancer cells as the primary tumor. Methods for reducing the risk of developing a metastasis in a subject having a cancer are described herein.

The phrase “risk of developing additional metastases” means the risk that a subject having a primary tumor and one or more additional tumors at sites distant from the primary tumor (where the one or more additional tumors include the same or similar cancer cells as the primary tumor) will develop one or more further tumors distant from the primary tumor, where the further tumors include the same or similar cancer cells as the primary tumor. Methods for reducing the risk of developing additional metastasis are described herein.

Some embodiments described herein provide methods of treating an autoimmune disorder (e.g., a MALT1-associated autoimmune disorder), such as rheumatoid arthritis, multiple sclerosis, and SLE, the method comprising administering an effective amount of a compound of Formula (I), or a pharmaceutically acceptable salt thereof, to a subject in need thereof.

Some embodiments described herein provide methods of treating an inflammatory disorder (e.g., a MALT1-associated autoimmune disorder), such as chronic graft versus host disease, the method comprising administering an effective amount of a compound of Formula (I), or a pharmaceutically acceptable salt thereof, to a subject in need thereof.

Also provided is a method for inhibiting MALT1 protease activity in a mammalian cell, comprising contacting the mammalian cell with a compound of Formula (I). In some embodiments, the contacting is in vitro. In some embodiments, the contacting is in vivo. In some embodiments, the contacting is in vivo, wherein the method comprises administering an effective amount of a compound of Formula (I), or a pharmaceutically acceptable salt thereof to a subject having a mammalian cell having MALT1 protease activity. In some embodiments, the mammalian cell is a mammalian immune cell. In some embodiments, the mammalian cell is a mammalian cancer cell. In some embodiments, the mammalian cancer cell is any cancer as described herein. In some embodiments, the mammalian cancer cell is a MALT1-associated mammalian cancer cell.

Also provided is a method for inhibiting MALT1 protease activity in a mammalian mammalian cell, comprising contacting the mammalian cell with a compound of Formula (I). In some embodiments, the contacting is in vitro. In some embodiments, the contacting is in vivo. In some embodiments, the contacting is in vivo, wherein the method comprises administering an effective amount of a compound of Formula (I), or a pharmaceutically acceptable salt thereof to a mammal having a mammalian cell having MALT1 protease activity. In some embodiments, the mammalian cell is a mammalian immune cell. In some embodiments, the mammalian cell is a mammalian cancer cell. In some embodiments, the mammalian cancer cell is any cancer as described herein. In some embodiments, the mammalian cancer cell is a MALT1-associated mammalian cancer cell. In some embodiments, the mammalian cell is a gastrointestinal mammalian cell.

As used herein, the term “contacting” refers to the bringing together of indicated moieties in an in vitro system or an in vivo system. For example, “contacting” a MALT1 protease with a compound provided herein includes the administration of a compound provided herein to a subject, such as a human, having a MALT1 protease, as well as, for example, introducing a compound provided herein into a sample containing a mammalian cellular or purified preparation containing the MALT1 protease.

Also provided herein is a method of inhibiting mammalian cell proliferation, in vitro or in vivo, the method comprising contacting a mammalian cell with an effective amount of a compound of Formula (I), or a pharmaceutically acceptable salt thereof, or a pharmaceutical composition thereof as defined herein.

A “MALT1 protease inhibitor” as defined herein includes any compound exhibiting MALT1 inhibition activity. In some embodiments, a MALT1 protease inhibitor is selective for a MALT1 protease. Exemplary MALT1 protease inhibitors can exhibit inhibition activity (IC₅₀) against a MALT1 protease of less than about 1000 nM, less than about 500 nM, less than about 200 nM, less than about 100 nM, less than about 50 nM, less than about 25 nM, less than about 10 nM, or less than about 1 nM as measured in an assay as described herein. In some embodiments, a MALT1 protease inhibitor can exhibit inhibition activity (IC₅₀) against a MALT1 protease of less than about 25 nM, less than about 10 nM, less than about 5 nM, or less than about 1 nM as measured in an assay as provided herein.

As used herein, a “first MALT1 protease inhibitor” or “first MALT1 inhibitor” is a MALT1 protease inhibitor as defined herein, but which does not include a compound of Formula (I), or a pharmaceutically acceptable salt thereof as defined herein. As used herein, a “second MALT1 protease inhibitor” or a “second MALT1 inhibitor” is a MALT1 protease inhibitor as defined herein, but which does not include a compound of Formula (I), or a pharmaceutically acceptable salt thereof as defined herein. When both a first and a second MALT1 inhibitor are present in a method provided herein, the first and second MALT1 protease inhibitor are different.

Exemplary first and second MALT1 protease inhibitors are described herein. In some embodiments, a first or second MALT1 protease inhibitor can be, for example, JNJ-67856633 or CTX-177 (ONO-7018)

The phrase “effective amount” means an amount of compound that, when administered to a subject in need of such treatment, is sufficient to (i) treat a MALT1-associated disease or disorder (such as a MALT1-associated cancer), (ii) attenuate, ameliorate, or eliminate one or more symptoms of the particular disease, condition, or disorder, or (iii) delay the onset of one or more symptoms of the particular disease, condition, or disorder described herein. The amount of a compound of Formula (I), or a pharmaceutically acceptable salt thereof that will correspond to such an amount will vary depending upon factors such as the particular compound, disease condition and its severity, the identity (e.g., weight) of the subject in need of treatment, but can nevertheless be routinely determined by one skilled in the art.

Another embodiment features a compound of Formula (I), or a pharmaceutically acceptable salt or pharmaceutical composition thereof, for use as a medicament.

Another embodiment features a compound of formula (I), or a pharmaceutically acceptable salt or pharmaceutical composition thereof, for use in a method of inhibiting MALT1 in a subject.

Another embodiment features a compound of Formula (I), or a pharmaceutically acceptable salt or pharmaceutical composition thereof, for use in a method of treating cancer, an autoimmune disorder, or an inflammatory disorder.

Other embodiments provide a compound of Formula (I), or a pharmaceutically acceptable salt or pharmaceutical composition thereof, for use in a method of treating an autoimmune disorder (e.g., a MALT1-associated autoimmune disorder) in a subject. In some embodiments, the autoimmune disorder is rheumatoid arthritis, multiple sclerosis, or systemic lupus erythematosus (SLE).

Some embodiments provide a compound of Formula (I), or a pharmaceutically acceptable salt or pharmaceutical composition thereof, for use in a method of treating an inflammatory disorder (e.g., a MALT1-associated inflammatory disorder) in a subject. In some embodiments, the inflammatory disorder is chronic graft versus host disease (cGVHD).

Some embodiments provide a compound of Formula (I), or a pharmaceutically acceptable salt or pharmaceutical composition thereof, for use in a method of treating cancer (e.g., a MALT1-associated cancer) in a subject. In some embodiments, the dysregulation of a MALT1 gene, a MALT1 protease, or the expression or activity or level of any of the same includes one or more fusion proteins.

Accordingly, also provided herein is a compound of Formula (I), or a pharmaceutically acceptable salt or pharmaceutical composition thereof, for use in a method for treating a subject diagnosed with or identified as having a MALT1-associated cancer, e.g., any of the exemplary MALT1-associated cancers disclosed herein.

Also provided herein is a compound of Formula (I), or a pharmaceutically acceptable salt of pharmaceutical composition thereof, for use in a method for treating diffuse large B-cell lymphoma (DLBCL), activated B-cell diffuse large B-cell lymphoma (ABC-DLBCL), mantel cell lymphoma (MCL), marginal zone lymphoma (MZL), chronic lymphatic leukemia (CLL) or mature B-cell neoplasms.

Also provided herein is a compound of Formula (I), or a pharmaceutically acceptable salt of pharmaceutical composition thereof, for use in a method for treating T-cell acute lymphoblastic leukemia.

Also provided herein is a compound of Formula (I), or a pharmaceutically acceptable salt or pharmaceutical composition thereof, for use in a method for treating a CBM complex pathway-associated cancer (such as any of those disclosed herein) in a subject.

Also provided herein is a compound of Formula (I), or a pharmaceutically acceptable salt or pharmaceutical composition thereof, for use in a method of treating a cancer in a subject identified as having a CBM complex pathway-associated cancer.

Also provided herein is a compound of Formula (I), or a pharmaceutically acceptable salt or pharmaceutical composition thereof, for use in a method of treating a MALT1-associated cancer in a subject.

Also provided herein is a compound of Formula (I), or a pharmaceutically acceptable salt or pharmaceutical composition thereof, for use in methods of treating CBM complex pathway-associated diseases or disorders, autoimmune disorders, and inflammatory disorders in a subject.

Accordingly, provided herein is a compound of Formula (I), or a pharmaceutically acceptable salt or pharmaceutical composition thereof, for use in a method for treating an autoimmune disorder in a subject. Also provided herein is a compound of Formula (I), or a pharmaceutically acceptable salt or pharmaceutical composition thereof, for use in a method of treating a MALT1-associated autoimmune disorder in a subject. Provided also herein is a compound of Formula (I), or a pharmaceutically acceptable salt or pharmaceutical composition thereof, for use in a method of treating a MALT1-associated autoimmune disorder in a subject.

In addition, provided herein is a compound of Formula (I), or a pharmaceutically acceptable salt or pharmaceutical composition thereof, for use in a method of treating an inflammatory disorder in a subject. In some cases, provided herein is a compound of Formula (I), or a pharmaceutically acceptable salt or pharmaceutical composition thereof, for use in a method of treating a MALT1-associated inflammatory disorder in a subject identified or diagnosed as having a MALT1-associated inflammatory disorder. Provided also herein is a compound of Formula (I), or a pharmaceutically acceptable salt or pharmaceutical composition thereof, for use in a method of treating a MALT1-associated inflammatory disorder in a subject determined to have a MALT1-associated inflammatory disorder.

Additionally provided herein is a compound of Formula (I), or a pharmaceutically acceptable salt or pharmaceutical composition thereof, for use in a method of treating a CBM complex pathway-associated disease or disorder in a subject. In addition, provided herein is a compound of Formula (I), or a pharmaceutically acceptable salt or pharmaceutical composition thereof, for use in a method of treating a disease or disorder in a subject identified as having a CBM complex pathway-associated disease or disorder.

Also provided herein is a compound of Formula (I), or a pharmaceutically acceptable salt or pharmaceutical composition thereof, for use in a method of inhibiting mammalian cell proliferation. Also provided herein is a compound of Formula (I), or a pharmaceutically acceptable salt or pharmaceutical composition thereof, for use in a method of inhibiting CBM complex pathway activity in a mammalian cell. Provided also herein is a compound of Formula (I), or a pharmaceutically acceptable salt or pharmaceutical composition thereof, for use in a method of inhibiting MALT1 protease activity in a mammalian cell. A mammalian cell can be any appropriate cell. In some embodiments, the mammalian cell is a mammalian immune cell. In some embodiments, the mammalian cell is a mammalian cancer cell. In some embodiments, the mammalian cancer cell is a mammalian CBM complex pathway-associated cancer cell. In some embodiments, the mammalian cancer cell is a mammalian MALT1-associated cancer cell. In some embodiments, the mammalian cell has dysregulation of a MALT1 gene, a MALT1 protease protein, or expression or activity or level of any of the same. In some embodiments, the dysregulation of a MALT1 gene, a MALT1 protease protein, or expression or activity or level of any of the same is an IAP2-MALT1 fusion, an IGH-MALT1 fusion, or a combination thereof.

Also provided herein is a compound of Formula (I), or a pharmaceutically acceptable salt or pharmaceutical composition thereof, for use in methods of inhibiting, preventing, aiding in the prevention, or decreasing the symptoms of metastasis of a cancer in a subject. Such methods can be used in the treatment of one or more of the cancers described herein. See, e.g., US Publication No. 2013/0029925; International Publication No. WO 2014/083567; and U.S. Pat. No. 8,568,998.

See also, e.g., Hezam K et al., Rev Neurosci 2018 Jan 26; 29:93-98; Gao L, et al., Pancreas 2015 Jan; 44:134-143; Ding K et al., J Biol Chemi 2014 Jun 6; 289:16057-71; and Amit M et al., Oncogene 2017 Jun 8; 36:3232-3239. In some embodiments, the cancer is a MALT1-associated cancer. In some embodiments, the compound of Formula (I), or a pharmaceutically acceptable salt thereof is used in combination with an additional therapy or another therapeutic agent, as described herein. For example, a first or second MALT1 protease inhibitor.

Also provided are a compound of Formula (I), or a pharmaceutically acceptable salt or pharmaceutical composition thereof, for use in methods of decreasing the risk of developing a metastasis or an additional metastasis in a subject having a MALT1-associated cancer. The decrease in the risk of developing a metastasis or an additional metastasis in a subject having a MALT1-associated cancer can be compared to the risk of developing a metastasis or an additional metastasis in the subject prior to treatment, or as compared to a subject or a population of subjects having a similar or the same MALT1-associated cancer that has received no treatment or a different treatment.

Some embodiments described herein provide a compound of Formula (I), or a pharmaceutically acceptable salt or pharmaceutical composition thereof, for use in methods of treating an autoimmune disorder (e.g., a MALT1-associated autoimmune disorder), such as rheumatoid arthritis, multiple sclerosis, and SLE in a subject.

Some embodiments described herein provide a compound of Formula (I), or a pharmaceutically acceptable salt or pharmaceutical composition thereof, for use in methods of treating an inflammatory disorder (e.g., a MALT1-associated autoimmune disorder), such as chronic graft versus host disease in a subject.

Also provided is a compound of Formula (I), or a pharmaceutically acceptable salt or pharmaceutical composition thereof, for use in a method of inhibiting MALT1 protease activity in a mammalian cell. In some embodiments, the mammalian cell is a mammalian immune cell. In some embodiments, the mammalian cell is a mammalian cancer cell. In some embodiments, the mammalian cancer cell is any cancer as described herein. In some embodiments, the mammalian cancer cell is a MALT1-associated mammalian cancer cell.

Also provided herein is a compound of Formula (I), or a pharmaceutically acceptable salt or pharmaceutical composition thereof, for use in a method of inhibiting mammalian cell proliferation.

In another aspect, the invention features a compound of Formula (I), or a pharmaceutically acceptable salt or pharmaceutical composition thereof, for use in a method wherein the subject is treated with one or more additional therapeutic agents administered concurrently with, prior to, or subsequent to treatment with an effective amount of the compound, pharmaceutically acceptable salt or pharmaceutical composition.

In another aspect, the invention provides the use of a compound of Formula (I), or a pharmaceutically acceptable salt or pharmaceutical composition thereof, for the manufacture of a medicament.

In another aspect, the invention provides the use of a compound of Formula (I), a pharmaceutically acceptable salt thereof, or a pharmaceutical composition thereof for the manufacture of a medicament for use in inhibiting MALT1 in a subject.

In yet another aspect, the invention provides the use of a compound of Formula (I), a pharmaceutically acceptable salt thereof, or a pharmaceutical composition thereof for the manufacture of a medicament for use in treating cancer, an autoimmune disorder, or an inflammatory disorder in a subject.

Some embodiments provide the use of a compound of Formula (I), or a pharmaceutically acceptable salt or pharmaceutical composition thereof, in the manufacture of a medicament for use in treating an autoimmune disorder (e.g., a MALT1-associated autoimmune disorder) in a subject.

In some embodiments, the autoimmune disorder is rheumatoid arthritis, multiple sclerosis, or systemic lupus erythematosus (SLE).

Some embodiments provide the use of a compound of Formula (I), or a pharmaceutically acceptable salt or pharmaceutical composition thereof, in the manufacture of a medicament for use in treating an inflammatory disorder (e.g., a MALT1-associated inflammatory disorder) in a subject. In some embodiments, the inflammatory disorder is chronic graft versus host disease (cGVHD).

Some embodiments provide the use of a compound of Formula (I), or a pharmaceutically acceptable salt or pharmaceutical composition thereof, in the manufacture of a medicament for use in treating cancer (e.g., a MALT1-associated cancer) in a subject. In some embodiments, the dysregulation of a MALT1 gene, a MALT1 protease, or the expression or activity or level of any of the same includes one or more fusion proteins.

Accordingly, also provided herein is the use of a compound of Formula (I), or a pharmaceutically acceptable salt or pharmaceutical composition thereof, in the manufacture of a medicament for use in treating a subject diagnosed with or identified as having a MALT1-associated cancer, e.g., any of the exemplary MALT1-associated cancers disclosed herein.

Accordingly, also provided herein is the use of a compound of Formula (I), or a pharmaceutically acceptable salt or pharmaceutical composition thereof, in the manufacture of a medicament for use in treating a subject with diffuse large B-cell lymphoma (DLBCL), activated B-cell diffuse large B-cell lymphoma (ABC-DLBCL), mantel cell lymphoma (MCL), marginal zone lymphoma (MZL), chronic lymphatic leukemia (CLL) or mature B-cell neoplasms.

Also provided herein is a compound of Formula (I), or a pharmaceutically acceptable salt of pharmaceutical composition thereof, in the manufacture of a medicament for use in treating a subject with T-cell acute lymphoblastic

Also provided herein is the use of a compound of Formula (I), or a pharmaceutically acceptable salt or pharmaceutical composition thereof, in the manufacture of a medicament for use in treating a CBM complex pathway-associated cancer (such as any of those disclosed herein) in a subject.

Also provided herein is the use of a compound of Formula (I), or a pharmaceutically acceptable salt or pharmaceutical composition thereof, in the manufacture of a medicament for use in treating a cancer in a subject identified as having a CBM complex pathway-associated cancer.

Also provided herein is the use of a compound of Formula (I), or a pharmaceutically acceptable salt or pharmaceutical composition thereof, in the manufacture of a medicament for use in treating a MALT1-associated cancer in a subject.

Also provided herein is the use of a compound of Formula (I), or a pharmaceutically acceptable salt or pharmaceutical composition thereof, in the manufacture of a medicament for use in treating CBM complex pathway-associated diseases or disorders, autoimmune disorders, and inflammatory disorders in a subject. Accordingly, provided herein is the use of a compound of Formula (I), or a pharmaceutically acceptable salt or pharmaceutical composition thereof, in the manufacture of a medicament for use in treating an autoimmune disorder in a subject. Also provided herein is the use of a compound of Formula (I), or a pharmaceutically acceptable salt or pharmaceutical composition thereof, in the manufacture of a medicament for use in treating a MALT1-associated autoimmune disorder in a subject. Provided also herein is the use of a compound of Formula (I), or a pharmaceutically acceptable salt or pharmaceutical composition thereof, in the manufacture of a medicament for use in treating a MALT1-associated autoimmune disorder in a subject. In addition, provided herein is the use of a compound of Formula (I), or a pharmaceutically acceptable salt or pharmaceutical composition thereof, in the manufacture of a medicament for use in treating an inflammatory disorder in a subject. In some cases, provided herein is the use of a compound of Formula (I), or a pharmaceutically acceptable salt or pharmaceutical composition thereof, in the manufacture of a medicament for use in treating a MALT1-associated inflammatory disorder in a subject identified or diagnosed as having a MALT1-associated inflammatory disorder. Provided also herein is the use of a compound of Formula (I), or a pharmaceutically acceptable salt or pharmaceutical composition thereof, in the manufacture of a medicament for use in treating a MALT1-associated inflammatory disorder in a subject determined to have a MALT1-associated inflammatory disorder.

Additionally provided herein is the use of a compound of Formula (I), or a pharmaceutically acceptable salt or pharmaceutical composition thereof, in the manufacture of a medicament for use in treating a CBM complex pathway-associated disease or disorder in a subject. In addition, provided herein is the use of a compound of Formula (I), or a pharmaceutically acceptable salt or pharmaceutical composition thereof, in the manufacture of a medicament for use in treating a disease or disorder in a subject identified as having a CBM complex pathway-associated disease or disorder.

Also provided herein is the use of a compound of Formula (I), or a pharmaceutically acceptable salt or pharmaceutical composition thereof, in the manufacture of a medicament for use in inhibiting mammalian cell proliferation. Also provided herein is the use of a compound of Formula (I), or a pharmaceutically acceptable salt or pharmaceutical composition thereof, in the manufacture of a medicament for use in inhibiting CBM complex pathway activity in a mammalian cell. Provided also herein is the use of a compound of Formula (I), or a pharmaceutically acceptable salt or pharmaceutical composition thereof, in the manufacture of a medicament for use in inhibiting MALT1 protease activity in a mammalian cell. A mammalian cell can be any appropriate cell. In some embodiments, the mammalian cell is a mammalian immune cell. In some embodiments, the mammalian cell is a mammalian cancer cell. In some embodiments, the mammalian cancer cell is a mammalian CBM complex pathway-associated cancer cell. In some embodiments, the mammalian cancer cell is a mammalian MALT1-associated cancer cell. In some embodiments, the mammalian cell has dysregulation of a MALT1 gene, a MALT1 protease protein, or expression or activity or level of any of the same. In some embodiments, the dysregulation of a MALT1 gene, a MALT1 protease protein, or expression or activity or level of any of the same is an IAP2-MALT1 fusion, an IGH-MALT1 fusion, or a combination thereof.

Also provided herein is the use of a compound of Formula (I), or a pharmaceutically acceptable salt or pharmaceutical composition thereof, in the manufacture of a medicament for use in inhibiting, preventing, aiding in the prevention, or decreasing the symptoms of metastasis of a cancer in a subject. In some embodiments, the cancer is a MALT1-associated cancer. In some embodiments, the compound of Formula (I), or a pharmaceutically acceptable salt thereof is used in combination with an additional therapy or another therapeutic agent, as described herein. For example, a first or second MALT1 protease inhibitor.

Also provided is the use of a compound of Formula (I), or a pharmaceutically acceptable salt or pharmaceutical composition thereof, in the manufacture of a medicament for use in decreasing the risk of developing a metastasis or an additional metastasis in a subject having a MALT1-associated cancer. The decrease in the risk of developing a metastasis or an additional metastasis in a subject having a MALT1-associated cancer can be compared to the risk of developing a metastasis or an additional metastasis in the subject prior to treatment, or as compared to a subject or a population of subjects having a similar or the same MALT1-associated cancer that has received no treatment or a different treatment.

Some embodiments described herein provide the use of a compound of Formula (I), or a pharmaceutically acceptable salt or pharmaceutical composition thereof, in the manufacture of a medicament for use in treating an autoimmune disorder (e.g., a MALT1-associated autoimmune disorder), such as rheumatoid arthritis, multiple sclerosis, and SLE in a subject.

Some embodiments described herein provide the use of a compound of Formula (I), or a pharmaceutically acceptable salt or pharmaceutical composition thereof, in the manufacture of a medicament for use in treating an inflammatory disorder (e.g., a MALT1-associated autoimmune disorder), such as chronic graft versus host disease in a subject.

Also provided is the use of a compound of Formula (I), or a pharmaceutically acceptable salt or pharmaceutical composition thereof, in the manufacture of a medicament for use in inhibiting MALT1 protease activity in a mammalian cell. In some embodiments, the mammalian cell is a mammalian immune cell. In some embodiments, the mammalian cell is a mammalian cancer cell. In some embodiments, the mammalian cancer cell is any cancer as described herein. In some embodiments, the mammalian cancer cell is a MALT1-associated mammalian cancer cell.

Also provided herein is the use of a compound of Formula (I), or a pharmaceutically acceptable salt or pharmaceutical composition thereof, in the manufacture of a medicament for use in inhibiting mammalian cell proliferation.

In yet another aspect, the invention provides the use of a compound of Formula (I), a pharmaceutically acceptable salt thereof, or a pharmaceutical composition thereof for the manufacture of a medicament for use in combination with one or more additional therapeutic agents administered concurrently with, prior to, or subsequent to treatment with the compound or pharmaceutical composition.

In the field of medical oncology, it is normal practice to use a combination of different forms of treatment to treat each subject with cancer. In medical oncology the other component(s) of such conjoint treatment or therapy in addition to compositions provided herein may be, for example, surgery, radiotherapy, and chemotherapeutic agents, such as other protease inhibitors, kinase inhibitors, signal transduction inhibitors, and/or monoclonal antibodies.

For example, a surgery may be open surgery or minimally invasive surgery. Compounds of Formula (I), or a pharmaceutically acceptable salt thereof therefore may also be useful as adjuvants to cancer treatment, that is, they can be used in combination with one or more additional therapies or therapeutic agents, for example, a chemotherapeutic agent that works by the same or by a different mechanism of action. In some embodiments, a compound of Formula (I), or a pharmaceutically acceptable salt thereof, can be used prior to administration of an additional therapeutic agent or additional therapy. For example, a subject in need thereof can be administered one or more doses of a compound of Formula (I), or a pharmaceutically acceptable salt thereof for a period of time and then undergo at least partial resection of the tumor. In some embodiments, the treatment with one or more doses of a compound of Formula (I), or a pharmaceutically acceptable salt thereof reduces the size of the tumor (e.g., the tumor burden) prior to the at least partial resection of the tumor. In some embodiments, a subject in need thereof can be administered one or more doses of a compound of Formula (T), or a pharmaceutically acceptable salt thereof for a period of time and under one or more rounds of radiation therapy. In some embodiments, the treatment with one or more doses of a compound of Formula (I), or a pharmaceutically acceptable salt thereof reduces the size of the tumor (e.g., the tumor burden) prior to the one or more rounds of radiation therapy.

In some embodiments, a subject has a cancer (e.g., a locally advanced or metastatic tumor) that is refractory or intolerant to standard therapy (e.g., administration of a chemotherapeutic agent), such as a first MALT1 inhibitor, a kinase inhibitor, immunotherapy, cell or gene therapy, or radiation (e.g., radioactive iodine). In some embodiments, a subject has a cancer (e.g., a locally advanced or metastatic tumor) that is refractory or intolerant to prior therapy (e.g., administration of a chemotherapeutic agent, such as a first MALT1 inhibitor or another protease inhibitor, immunotherapy, cell or gene therapy, or radiation (e.g., radioactive iodine). In some embodiments, a subject has a cancer (e.g., a locally advanced or metastatic tumor) that has no standard therapy. In some embodiments, a subject is MALT1-protease inhibitor naive. For example, the subject is naive to treatment with a selective MALT1-protease inhibitor. In some embodiments, a subject is not MALT1-protease inhibitor naive.

In some embodiments of any of the methods described herein, the compound of Formula (I), or a pharmaceutically acceptable salt thereof, is administered in combination with an effective amount of at least one additional therapeutic agent selected from one or more additional therapies or therapeutic (e.g., chemotherapeutic or immunomodulatory) agents. An additional therapy or therapeutic agent can be any appropriate additional therapy or therapeutic agent, such as any of those described herein.

Non-limiting examples of additional therapeutic agents include: other MALT1-targeted therapeutic agents (i.e. a first or second MALT1 protease inhibitor, e.g., JNJ-67856633 or CTX-177 (ONO-7018)), other protease inhibitors, kinase inhibitors (e.g., receptor tyrosine kinase-targeted therapeutic agents such as BTK or EGFR inhibitors), signal transduction pathway inhibitors, checkpoint inhibitors, modulators of the apoptosis pathway (e.g., venetoclax or obataclax); cytotoxic chemotherapeutics, angiogenesis-targeted therapies, immune-targeted agents (including antibody and cell-based immunotherapies, and antibody-drug conjugates) and radiotherapy.

In some embodiments, the compound of Formula (I), or a pharmaceutically acceptable salt thereof, and the additional therapeutic agent are administered simultaneously as separate dosages.

In some embodiments, the compound of Formula (I), or a pharmaceutically acceptable salt thereof, and the additional therapeutic agent are administered as separate dosages sequentially in any order.

In some embodiments, the other MALT1-targeted therapeutic is another protease inhibitor exhibiting MALT1 inhibition activity. In some embodiments, the other MALT1-targeted therapeutic inhibitor is selective for a MALT1 protease. Exemplary MALT1 protease inhibitors can exhibit inhibition activity (IC₅₀) against a MALT1 protease of less than about 1000 nM, less than about 500 nM, less than about 200 nM, less than about 100 nM, less than about 50 nM, less than about 25 nM, less than about 10 nM, or less than about 1 nM as measured in an assay as described herein. In some embodiments, a MALT1 protease inhibitors can exhibit inhibition activity (IC₅₀) against a MALT1 protease of less than about 25 nM, less than about 10 nM, less than about 5 nM, or less than about 1 nM as measured in an assay as provided herein.

Non-limiting examples of protease-targeted therapeutic agents (e.g., a first MALT1 inhibitor or a second MALT1 inhibitor) include JNJ-67856633 and CTX-177 (ONO-7018).

Non-limiting examples of multi-kinase inhibitors include alectinib (9-Ethyl-6,6-dimethyl-8-[4-(morpholin-4-yl)piperidin-1-yl]-11-oxo-6,11-dihydro-5H-benzo[b]carbazole-3-carbonitrile); amuvatinib (MP470, HPK56) (N-(1,3-benzodioxol-5-ylmethyl)-4-([1]benzofuro[3,2-d]pyrimidin-4-yl)piperazine-1-carbothioamide); apatinib (YN968D1) (N-[4-(1-cyanocyclopentyl) phenyl-2-(4-picolyl)amino-3-Nicotinamide methanesulphonate); cabozantinib (Cometriq XL-184) (N-(4-((6,7-Dimethoxyquinolin-4-yl)oxy)phenyl)-N′-(4-fluorophenyl)cyclopropane-1,1-dicarboxamide); dovitinib (TK1258; GFKI-258; CHIR-258) ((3Z)-4-amino-5-fluoro-3-[5-(4-methylpiperazin-1-yl)-1,3-dihydrobenzimidazol-2-ylidene]quinolin-2-one); famitinib (5-[2-(diethylamino)ethyl]-2-[(Z)-(5-fluoro-2-oxo-1H-indol-3-ylidene)methyl]-3-methyl-6,7-dihydro-1H-pyrrolo[3,2-c]pyridin-4-one); fedratinib (SAR302503, TG101348) (N-(2-Methyl-2-propanyl)-3-{[5-methyl-2-({4-[2-(1-pyrrolidinyl)ethoxy]phenyl}amino)-4-pyrimidinyl]amino}benzenesulfonamide); foretinib (XL880, EXEL-2880, GSK1363089, GSK089) (N1′-[3-fluoro-4-[[6-methoxy-7-(3-morpholinopropoxy)-4-quinolyl]oxy]phenyl]-N1-(4-fluorophenyl)cyclopropane-1,1-dicarboxamide); fostamantinib (R788) (2H-Pyrido[3,2-b]-1,4-oxazin-3(4H)-one, 6-[[5-fluoro-2-[(3,4,5-trimethoxyphenyl)amino]-4-pyrimidinyl]amino]-2,2-dimethyl-4-[(phosphonooxy)methyl]-, sodium salt (1:2)); ilorasertib (ABT-348) (1-(4-(4-amino-7-(1-(2-hydroxyethyl)-1H-pyrazol-4-yl)thieno[3,2-c]pyridin-3-yl)phenyl)-3-(3-fluorophenyl)urea); lenvatinib (E7080, Lenvima) (4-[3-chloro-4-(cyclopropylaminocarbonyl)aminophenoxy]-7-methoxy-6-quinolinecarboxamide); motesanib (AMG 706) (N-(3,3-Dimethyl-2,3-dihydro-1H-indol-6-yl)-2-[(pyridin-4-ylmethyl)amino]pyridine-3-carboxamide); nintedanib (3-Z-[1-(4-(N-((4-methyl-piperazin-1-yl)-methylcarbonyl)-N-methyl-amino)-anilino)-1-phenyl-methylene]-6-methyoxycarbonyl-2-indolinone); ponatinib (AP24534) (3-(2-Imidazo[1,2-b]pyridazin-3-ylethynyl)-4-methyl-N-[4-[(4-methylpiperazin-1-yl)methyl]-3-(trifluoromethyl)phenyl]benzamide); PP242 (torkinib) (2-[4-Amino-1-(1-methylethyl)-1H-pyrazolo[3,4-d]pyrimidin-3-yl]-1H-indol-5-ol); quizartinib (1-(5-(tert-Butyl)isoxazol-3-yl)-3-(4-(7-(2-morpholinoethoxy)benzo[d]imidazo[2,1-b]thiazol-2-yl)phenyl)urea); regorafenib (BAY 73-4506, stivarga) (4-[4-({[4-Chloro-3-(trifluoromethyl)phenyl]carbamoyl}amino)-3-fluorophenoxy]-N-methylpyridine-2-carboxamide hydrate); RXDX-105 (CEP-32496, agerafenib) (1-(3-((6,7-dimethoxyquinazolin-4-yl)oxy)phenyl)-3-(5-(1,1,1-trifluoro-2-methylpropan-2-yl)isoxazol-3-yl)urea); semaxanib (SU5416) ((3Z)-3-[(3,5-dimethyl-1H-pyrrol-2-yl)methylidene]-1,3-dihydro-2H-indol-2-one); sitravatinib (MGCD516, MG516) (N-(3-Fluoro-4-{[2-(5-{[(2-methoxyethyl)amino]methyl}-2-pyridinyl)thieno[3,2-b]pyridin-7-yl]oxy}phenyl)-N′-(4-fluorophenyl)-1,1-cyclopropanedicarboxamide); sorafenib (BAY 43-9006) (4-[4-[[[[4-chloro-3-(trifluoromethyl)phenyl]amino]carbonyl]amino]phenoxy]-N-methyl-2-pyridinecarboxamide); vandetanib (N-(4-bromo-2-fluorophenyl)-6-methoxy-7-[(1-methylpiperidin-4-yl)methoxy]quinazolin-4-amine); vatalanib (PTK787, PTK/ZK, ZK222584) (N-(4-chlorophenyl)-4-(pyridin-4-ylmethyl)phthalazin-1-amine); AD-57 (N-[4-[4-amino-1-(1-methylethyl)-1H-pyrazolo[3,4-d]pyrimidin-3-yl]phenyl]-N′-[3-(trifluoromethyl)phenyl]-urea); AD-80 (1-[4-(4-amino-1-propan-2-ylpyrazolo[3,4-d]pyrimidin-3-yl)phenyl]-3-[2-fluoro-5-(trifluoromethyl)phenyl]urea); AD-81 (1-(4-(4-amino-1-isopropyl-1H-pyrazolo[3,4-d]pyrimidin-3-yl)phenyl)-3-(4-chloro-3-(trifluoromethyl)phenyl)urea); ALW-II-41-27 (N-(5-((4-((4-ethylpiperazin-1-yl)methyl)-3-(trifluoromethyl)phenyl)carbamoyl)-2-methylphenyl)-5-(thiophen-2-yl)nicotinamide); BPR1K871 (1-(3-chlorophenyl)-3-(5-(2-((7-(3-(dimethylamino)propoxy)quinazolin-4-yl)amino)ethyl)thiazol-2-yl)urea); CLM3 (1-phenethyl-N-(1-phenylethyl)-1H-pyrazolo[3,4-d]pyrimidin-4-amine); EBI-907 (N-(2-chloro-3-(1-cyclopropyl-8-methoxy-3H-pyrazolo[3,4-c]isoquinolin-7-yl)-4-fluorophenyl)-3-fluoropropane-1-sulfonamide); NVP-AST-487 (N-[4-[(4-ethyl-1-piperazinyl)methyl]-3-(trifluoromethyl)phenyl]-N′-[4-[[6-(methylamino)-4-pyrimidinyl]oxy]phenyl]-urea); NVP-BBT594 (BBT594) (5-((6-acetamidopyrimidin-4-yl)oxy)-N-(4-((4-methylpiperazin-1-yl)methyl)-3-(trifluoromethyl)phenyl)indoline-1-carboxamide); PD173955 (6-(2,6-dichlorophenyl)-8-methyl-2-(3-methylsulfanylanilino)pyrido[2,3-d]pyrimidin-7-one); PP2 (4-amino-5-(4-chlorophenyl)-7-(dimethylethyl)pyrazolo[3,4-d]pyrimidine); PZ-1 (N-(5-(tert-butyl)isoxazol-3-yl)-2-(4-(5-(1-methyl-1H-pyrazol-4-yl)-1Hbenzo[d]imidazol-1-yl)phenyl)acetamide); RPI-1 (1,3-dihydro-5,6-dimethoxy-3-[(4-hydroxyphenyl)methylene]-H-indol-2-one; (3E)-3-[(4-hydroxyphenyl)methylidene]-5,6-dimethoxy-1H-indol-2-one); SGI-7079 (3-[2-[[3-fluoro-4-(4-methyl-1-piperazinyl)phenyl]amino]-5-methyl-7H-pyrrolo[2,3-d]pyrimidin-4-yl]-benzeneacetonitrile); SPP86 (1-Isopropyl-3-(phenylethynyl)-1H-pyrazolo[3,4-d]pyrimidin-4-amine); SU4984 (4-[4-[(E)-(2-oxo-1H-indol-3-ylidene)methyl]phenyl]piperazine-1-carbaldehyde); sunitinb (SU11248) (N-(2-Diethylaminoethyl)-5-[(Z)-(5-fluoro-2-oxo-1H-indol-3-ylidene)methyl]-2,4-dimethyl-1H-pyrrole-3-carboxamide); TG101209 (N-tert-butyl-3-(5-methyl-2-(4-(4-methylpiperazin-1-yl)phenylamino)pyrimidin-4-ylamino)benzenesulfonamide); Withaferin A ((4β,5β,6β,22R)-4,27-Dihydroxy-5,6:22,26-diepoxyergosta-2,24-diene-1,26-dione); XL-999 ((Z)-5-((1-ethylpiperidin-4-yl)amino)-3-((3-fluorophenyl)(5-methyl-1H-imidazol-2-yl)methylene)indolin-2-one); BPR1J373 (a 5-phenylthiazol-2-ylamine-pyriminide derivative); CG-806 (CG'806); DCC-2157; GTX-186; HG-6-63-01 ((E)-3-(2-(4-chloro-1H-pyrrolo[2,3-b]pyridin-5-yl)vinyl)-N-(4-((4-ethylpiperazin-1-yl)methyl)-3-(trifluoromethyl)phenyl)-4-methylbenzamide); SW-01 (Cyclobenzaprine hydrochloride); XMD15-44 (N-(4-((4-ethylpiperazin-1-yl)methyl)-3-(trifluoromethyl)phenyl)-4-methyl-3-(pyridin-3-ylethynyl)benzamide (generated from structure)); ITRI-305 (DONSTB, DIB003599); BLU-667 ((1S,4R)—N—((S)-1-(6-(4-fluoro-1H-pyrazol-1-yl)pyridin-3-yl)ethyl)-1-methoxy-4-(4-methyl-6-((5-methyl-1H-pyrazol-3-yl)amino)pyrimidin-2-yl)cyclohexane-1-carboxamide); BLU6864; DS-5010; GSK3179106; GSK3352589; NMS-E668; TAS0286/HM05; TPX0046; and N-(3-(2-(dimethylamino)ethoxy)-5-(trifluoromethyl)phenyl)-2-(4-(4-ethoxy-6-oxo-1,6-dihydropyridin-3-yl)-2-fluorophenyl)acetamide.

Non-limiting examples of receptor tyrosine kinase (e.g., Trk) targeted therapeutic agents, include afatinib, cabozantinib, cetuximab, crizotinib, dabrafenib, entrectinib, erlotinib, gefitinib, imatinib, lapatinib, lestaurtinib, nilotinib, pazopanib, panitumumab, pertuzumab, sunitinib, trastuzumab, 1-((3S,4R)-4-(3-fluorophenyl)-1-(2-methoxyethyl)pyrrolidin-3-yl)-3-(4-methyl-3-(2-methylpyrimidin-5-yl)-1-phenyl-1H-pyrazol-5-yl)urea, AG 879, AR-772, AR-786, AR-256, AR-618, AZ-23, AZ623, DS-6051, Go 6976, GNF-5837, GTx-186, GW 441756, LOXO-101, MGCD516, PLX7486, RXDX101, VM-902A, TPX-0005, TSR-011, GNF-4256, N-[3-[[2,3-dihydro-2-oxo-3-(1H-pyrrol-2-ylmethylene)-1H-indol-6-yl]amino]-4-methylphenyl]-N′-[2-fluoro-5-(trifluoromethyl)phenyl]-urea, AZ623, AZ64, (S)-5-Chloro-N2-(1-(5-fluoropyridin-2-yl)ethyl)-N4-(5-isopropoxy-1H-pyrazol-3-yl)pyrimidine-2,4-diamine, AZD7451, CEP-751, CT327, sunitinib, GNF-8625, and (R)-1-(6-(6-(2-(3-fluorophenyl)pyrrolidin-1-yl)imidazo[1,2-b]pyridazin-3-yl)-[2,4′-bipyridin]-2′-yl)piperidin-4-ol.

In some embodiments, the additional therapeutic agent is a BRAF inhibitor. Non-limiting examples of a BRAF inhibitor include dabrafenib, vemurafenib (also called RG7204 or PLX4032), sorafenib tosylate, PLX-4720, GDC-0879, BMS-908662 (Bristol-Meyers Squibb), LGX818 (Novartis), PLX3603 (Hofmann-LaRoche), RAF265 (Novartis), RO5185426 (Hofmann-LaRoche), and GSK2118436 (GlaxoSmithKline). Additional examples of a BRAF inhibitor are known in the art.

In some embodiments, the additional therapeutic agent is an epidermal growth factor receptor typrosine kinase inhibitor (EGFR). For example, EGFR inhibitors can include osimertinib (merelectinib, Tagrisso), erlotinib (Tarceva), gefitinib (Iressa), cetuximab (Erbitux), necitumumab (Portrazza), neratinib (Nerlynx), lapatinib (Tykerb), panitumumab (Vectibix), and vandetanib (Caprelsa).

In some embodiments, the additional therapeutic agent is a Ras-Raf-MEK-ERK pathway inhibitors (e.g., binimetinib, selumetinib, encorafenib, sorafenib, trametinib, and vemurafenib), PI3K-Akt-mTOR-S6K pathway inhibitors (e.g., everolimus, rapamycin, perifosine, temsirolimus), and other kinase inhibitors, such as baricitinib, brigatinib, capmatinib, danusertib, ibrutinib, milciclib, quercetin, regorafenib, ruxolitinib, semaxanib, AP32788, BLU285, BLU554, INCB39110, INCB40093, INCB50465, INCB52793, INCB54828, MGCD265, NMS-088, NMS-1286937, PF 477736 ((R)-amino-N-[5,6-dihydro-2-(1-methyl-1H-pyrazol-4-yl)-6-oxo-1Hpyrrolo[4,3,2-ef][2,3]benzodiazepin-8-yl]-cyclohexaneacetamide), PLX3397, PLX7486, PLX8394, PLX9486, PRN1008, PRN1371, RXDX103, RXDX106, RXDX108, and TG101209 (N-tert-butyl-3-(5-methyl-2-(4-(4-methylpiperazin-1-yl)phenylamino)pyrimidin-4-ylamino)benzenesulfonamide).

In some embodiments, the additional therapeutic agent is a BTK inhibitor. Non-limiting examples of BTK inhibitors include ibrutinib, acalabrutinib, zanubrutinib, pirtobrutinib, and NX-2127.

In some embodiments, the additional therapeutic agent is a Bcl-2 inhibitor. Non-limiting examples of Bcl-2 inhibitors include venetoclax, navitoclax, oblimersen, obatoclax, and AT-101.

In some embodiments, the additional therapeutic agent is a PI3K inhibitor. Non-limiting examples of PI3K inhibitors include idelalisib, copanlisib, duvelisib, alpelisib, taselisib, buparlisib, umbralisib, and copanlisib.

In some embodiments, the additional therapeutic agent is a mTOR inhibitor. Non-limiting examples of mTOR inhibitors include everolimus, temsirolimus, and ridaforolimus.

In some embodiments, the additional therapeutic agent is a HDAC inhibitor. Non-limiting examples of HDAC inhibitors include vorinostat, romidepsin, belinostat, chidamide, panobinostat, CXD101, and abexinostat.

In some embodiments, the additional therapeutic agent is a checkpoint inhibitor. Non-limiting examples of checkpoint inhibitors include ipilimumab, tremelimumab, nivolumab, pidilizumab, MPDL3208A, MEDI4736, MSB0010718C, BMS-936559, BMS-956559, BMS-935559 (MDX-1105), AMP-224, and pembrolizumab.

In some embodiments, the additional therapeutic agent is a cytotoxic chemotherapeutic. Non-limiting example of cytotoxic chemotherapeutics include arsenic trioxide, bleomycin, bendamustine, cabazitaxel, capecitabine, carboplatin, cisplatin, cyclophosphamide, cytarabine, dacarbazine, daunorubicin, docetaxel, doxorubicin, etoposide, fluorouracil, gemcitabine, irinotecan, lomustine, methotrexate, mitomycin C, oxaliplatin, paclitaxel, pemetrexed, temozolomide, and vincristine.

In some embodiments, the additional therapeutic agent is an angiogenesis-targeted therapeutic. Non-limiting examples of angiogenesis-targeted therapies include lenalidomide, enzastaurine, aflibercept, and bevacizumab.

In some embodiments, an additional therapy or therapeutic agent can include a histidyl-tRNA synthetase (HRS) polypeptide or an expressible nucleotide that encodes the HRS polypeptide.

The term “immunotherapy” refers to an agent that modulates the immune system. In some embodiments, an immunotherapy can increase the expression and/or activity of a regulator of the immune system. In some embodiments, an immunotherapy can decrease the expression and/or activity of a regulator of the immune system. In some embodiments, an immunotherapy can recruit and/or enhance the activity of an immune cell.

In some embodiments, the immunotherapy is a cellular immunotherapy (e.g., adoptive T-cell therapy, dendritic cell therapy, natural killer cell therapy). In some embodiments, the cellular immunotherapy is sipuleucel-T (APC8015; Provenge™; Plosker (2011) Drugs 71(1): 101-108). In some embodiments, the cellular immunotherapy includes cells that express a chimeric antigen receptor (CAR). In some embodiments, the cellular immunotherapy is a CAR-T cell therapy. In some embodiments, the CAR-T cell therapy is tisagenlecleucel (Kymria). In some embodiments, the CAR-T cell therapy is axicabtagene ciloleucel (Yescarta). In some embodiments, the CAR-T cell therapy is brexucabtagene autoleucel (Tecartus). In some embodiments, the CAR-T cell therapy is relmacabtagene autoleucel. In some embodiments, the CAR-T cell therapy is ALLO-501.

In some embodiments, the immunotherapy is an antibody therapy (e.g., a monoclonal antibody, a conjugated antibody, or a bispecific antibody). In some embodiments, the antibody therapy is bevacizumab (Mvasti™, Avastin®), trastuzumab (Herceptin®), avelumab (Bavencio®), rituximab (MabThera™, Rituxan®), rituximab with human hyaluronidase (Rituxan Hycela™), edrecolomab (Panorex), daratumuab (Darzalex®), olaratumab (Lartruvo™) ofatumumab (Arzerra®), alemtuzumab (Campath®), cetuximab (Erbitux®), oregovomab, pembrolizumab (Keytruda®), dinutiximab (Unituxin®), obinutuzumab (Gazyva®), tremelimumab (CP-675,206), ramucirumab (Cyramza®), ublituximab (TG-1101), panitumumab (Vectibix®), elotuzumab (Empliciti™), avelumab (Bavencio®), necitumumab (Portrazza™) cirmtuzumab (UC-961), ibritumomab (Zevalin®), isatuximab (SAR650984), nimotuzumab, fresolimumab (GC1008), lirilumab (INN), mogamulizumab (Poteligeo®), ficlatuzumab (AV-299), denosumab (Xgeva®), lenzilumab, avelumab, spartalizumab, pembrolizumab, utomilumab, ublituximab, blinatumomab ganitumab, urelumab, pidilizumab, amatuximab, mosunetuzumab (BTCT4465A), CD20-TCB, RO7082859, XmAb13676, glofitamab, CD20-TDB, odronextamab (REGN1979), IGM-2323, BTCT4465A, AMG-562, or TTI-621.

In some embodiments, the immunotherapy is an antibody-drug conjugate. In some embodiments, the antibody-drug conjugate is gemtuzumab ozogamicin (Mylotarg™), inotuzumab ozogamicin (Besponsa®), brentuximab vedotin (Adcetris®), ado-trastuzumab emtansine (TDM-1; Kadcyla®), mirvetuximab soravtansine (IMGN853), anetumab ravtansine, polatuzumab vedotine, loncastuximab tesirine (ADCT-402), camidanlumab tesirine (ADCT-301), or naratuximab emtansine (Debio 1562).

In some embodiments, the immunotherapy includes blinatumomab (AMG103; Blincyto®) or midostaurin (Rydapt).

In some embodiments, the immunotherapy includes a toxin. In some embodiments, the immunotherapy is denileukin diftitox (Ontak®).

In some embodiments, the immunotherapy is a cytokine therapy. In some embodiments, the cytokine therapy is an interleukin 2 (IL-2) therapy, an interferon alpha (IFNα) therapy, a granulocyte colony stimulating factor (G-CSF) therapy, an interleukin 12 (IL-12) therapy, an interleukin 15 (IL-15) therapy, an interleukin 7 (IL-7) therapy or an erythropoietin-alpha (EPO) therapy. In some embodiments, the IL-2 therapy is aldesleukin (Proleukin®). In some embodiments, the IFNα therapy is IntronA® (Roferon-A®). In some embodiments, the G-CSF therapy is filgrastim (Neupogen®).

In some embodiments, the immunotherapy is an immune checkpoint inhibitor. In some embodiments, the immunotherapy includes one or more immune checkpoint inhibitors. In some embodiments, the immune checkpoint inhibitor is a CTLA-4 inhibitor, a PD-1 inhibitor or a PD-L1 inhibitor. In some embodiments, the CTLA-4 inhibitor is ipilimumab (Yervoy®) or tremelimumab (CP-675,206). In some embodiments, the PD-1 inhibitor is pembrolizumab (Keytruda®) or nivolumab (Opdivo®). In some embodiments, the PD-L1 inhibitor is atezolizumab (Tecentriq®), avelumab (Bavencio®) or durvalumab (Imfinzi™)

In some embodiments, the immunotherapy is mRNA-based immunotherapy. In some embodiments, the mRNA-based immunotherapy is CV9104 (see, e.g., Rausch et al. (2014) Human Vaccin Immunother 10(11): 3146-52; and Kubler et al. (2015) J. Immunother Cancer 3:26).

In some embodiments, the immunotherapy is bacillus Calmette-Guerin (BCG) therapy.

In some embodiments, the immunotherapy is an oncolytic virus therapy. In some embodiments, the oncolytic virus therapy is talimogene alherparepvec (T-VEC; Imlygic®).

In some embodiments, the immunotherapy is a cancer vaccine. In some embodiments, the cancer vaccine is a human papillomavirus (HPV) vaccine. In some embodiments, the HPV vaccine is Gardasil®, Gardasil9® or Cervarix®. In some embodiments, the cancer vaccine is a hepatitis B virus (HBV) vaccine. In some embodiments, the HBV vaccine is Engerix-B®, Recombivax HB® or GI-13020 (Tarmogen®). In some embodiments, the cancer vaccine is Twinrix® or Pediarix®.

In some embodiments, the cancer vaccine is BiovaxID®, Oncophage®, GVAX, ADXS11-001, ALVAC-CEA, PROSTVAC®, Rindopepimut®, CimaVax-EGF, lapulcuccl-T (APC8024; Neuvenge™), GRNVAC1, GRNVAC2, GRN-1201, hepcortespenlisimut-L (Hepko-V5), DCVAX®, SCIB1, BMT CTN 1401, PrCa VBIR, PANVAC, ProstAtak®, DPX-Survivac, or viagenpumatucel-L (HS-110).

In some embodiments, the immunotherapy is a peptide vaccine. In some embodiments, the peptide vaccine is nelipepimut-S (E75) (NeuVax™), IMA901, or SurVaxM (SVN53-67). In some embodiments, the cancer vaccine is an immunogenic personal neoantigen vaccine (see, e.g., Ott et al. (2017) Nature 547: 217-221; Sahin et al. (2017) Nature 547: 222-226). In some embodiments, the cancer vaccine is RGSH4K, or NEO-PV-01. In some embodiments, the cancer vaccine is a DNA-based vaccine. In some embodiments, the DNA-based vaccine is a mammaglobin-A DNA vaccine (see, e.g., Kim et al. (2016) Oncolmmunology 5(2): e1069940).

In some embodiments, immune-targeted agents are selected from aldesleukin, interferon alfa-2b, ipilimumab, lambrolizumab, nivolumab, prednisone, and sipuleucel-T.

In some embodiments, the additional therapy is radiotherapy. Non-limiting examples of radiotherapy include radioiodide therapy, external-beam radiation, and radium 223 therapy.

In some embodiments, the additional therapeutic agent is GSK-3368715, PF-06821497, ceralasertib; AZD6738, BI-894999, MAK-683, AZD-6738, taminadenant, TAK-981, MIK-665, or danvatirsen.

Additional kinase inhibitors include those described in, for example, U.S. Pat. Nos. 7,514,446; 7,863,289; 8,026,247; 8,501,756; 8,552,002; 8,815,901; 8,912,204; 9,260,437; 9,273,051; U.S. Publication No. US 2015/0018336; International Publication No. WO 2007/002325; WO 2007/002433; WO 2008/080001; WO 2008/079906; WO 2008/079903; WO 2008/079909; WO 2008/080015; WO 2009/007748; WO 2009/012283; WO 2009/143018; WO 2009/143024; WO 2009/014637; 2009/152083; WO 2010/111527; WO 2012/109075; WO 2014/194127; WO 2015/112806; WO 2007/110344; WO 2009/071480; WO 2009/118411; WO 2010/031816; WO 2010/145998; WO 2011/092120; WO 2012/101032; WO 2012/139930; WO 2012/143248; WO 2012/152763; WO 2013/014039; WO 2013/102059; WO 2013/050448; WO 2013/050446; WO 2014/019908; WO 2014/072220; WO 2014/184069; WO 2016/075224; WO 2016/081450; WO 2016/022569; WO 2016/011141; WO 2016/011144; WO 2016/011147; WO 2015/191667; WO 2012/101029; WO 2012/113774; WO 2015/191666; WO 2015/161277; WO 2015/161274; WO 2015/108992; WO 2015/061572; WO 2015/058129; WO 2015/057873; WO 2015/017528; WO/2015/017533; WO 2014/160521; and WO 2014/011900, each of which is hereby incorporated by reference in its entirety.

In some embodiments, the subject was previously administered one or more standard of care therapies for a lymphoma. In some embodiments, the previously administered standard of care therapy is polatuzumab vedotine, selinexor, axicabtagene ciloleucel (Yescarta), tisagenlecleucel (Kymriah), bendamustine in combination with rituximab and polatuzumab vedotin, tafasitamab in combination with lenalidomide, or rituximab with human hyaluronidase (Rituxan Hycela).

In some embodiments, the subject is concomitantly receiving standard of care therapy for a lymphoma. In some embodiments, the standard of care therapy is polatuzumab vedotine, selinexor, axicabtagene ciloleucel (Yescarta), tisagenlecleucel (Kymriah), bendamustine in combination with rituximab and polatuzumab vedotin, tafasitamab in combination with lenalidomide, or rituximab with human hyaluronidase (Rituxan Hycela).

### Pharmaceutical Compositions

When employed as pharmaceuticals, compounds of Formula (I), including pharmaceutically acceptable salts thereof, can be administered in the form of pharmaceutical compositions. These compositions can be prepared in a manner well known in the pharmaceutical art, and can be administered by a variety of routes, depending upon whether local or systemic treatment is desired and upon the area to be treated. Administration can be topical (including transdermal, epidermal, ophthalmic and to mucous membranes including intranasal, vaginal and rectal delivery), pulmonary (e.g., by inhalation or insufflation of powders or aerosols, including by nebulizer; intratracheal or intranasal), oral or parenteral. Oral administration can include a dosage form formulated for once-daily or twice-daily (BID) administration. Parenteral administration includes intravenous, intraarterial, subcutaneous, intraperitoneal intramuscular or injection or infusion; or intracranial, e.g., intrathecal or intraventricular, administration. Parenteral administration can be in the form of a single bolus dose, or can be, for example, by a continuous perfusion pump. Pharmaceutical compositions and formulations for topical administration can include transdermal patches, ointments, lotions, creams, gels, drops, suppositories, sprays, liquids and powders. Conventional pharmaceutical carriers, aqueous, powder or oily bases, thickeners and the like may be necessary or desirable.

Also provided herein are pharmaceutical compositions which contain, as the active ingredient, a compound of Formula (I) or pharmaceutically acceptable salt thereof, in combination with one or more pharmaceutically acceptable excipients. For example, a pharmaceutical composition prepared using a compound of Formula (I) or a pharmaceutically acceptable salt thereof. In some embodiments, the composition is suitable for topical administration. In making the compositions provided herein, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders. In some embodiments, the composition is formulated for oral administration. In some embodiments, the composition is a solid oral formulation. In some embodiments, the composition is formulated as a tablet or capsule.

Further provided herein are pharmaceutical compositions containing a compound of Formula (I) or a pharmaceutically acceptable salt thereof with a pharmaceutically acceptable carrier. Pharmaceutical compositions containing a compound of Formula (I) or a pharmaceutically acceptable salt thereof as the active ingredient can be prepared by intimately mixing the compound of Formula (I), or a pharmaceutically acceptable salt thereof with a pharmaceutical carrier according to conventional pharmaceutical compounding techniques. The carrier can take a wide variety of forms depending upon the desired route of administration (e.g., oral. parenteral). In some embodiments, the composition is a solid oral composition.

Suitable pharmaceutically acceptable carriers are well known in the art. Descriptions of some of these pharmaceutically acceptable carriers can be found in The Handbook of Pharmaceutical Excipients, published by the American Pharmaceutical Association and the Pharmaceutical Society of Great Britain.

Methods of formulating pharmaceutical compositions have been described in numerous publications such as Pharmaceutical Dosage Forms: Tablets, Second Edition, Revised and Expanded, Volumes 1-3, edited by Lieberman et al; Pharmaceutical Dosage Forms: Parenteral Medications, Volumes 1-2, edited by Avis et al; and Pharmaceutical Dosage Forms: Disperse Systems, Volumes 1-2, edited by Lieberman et al; published by Marcel Dekker, Inc.

In preparing the compositions in oral dosage form, any of the usual pharmaceutical media can be employed. Thus for liquid oral preparations such as suspensions, elixirs and solutions, suitable carriers and additives include water, glycols, oils, alcohols, flavoring agents, preservatives, stabilizers, coloring agents and the like; for solid oral preparations, such as powders, capsules and tablets, suitable carriers and additives include starches, sugars, diluents, granulating agents, lubricants, binders, disintegrating agents and the like. Suitable binders include, without limitation, starch, gelatin, natural sugars such as glucose or beta-lactose, corn sweeteners, natural and synthetic gums such as acacia, tragacanth or sodium oleate, sodium stearate, magnesium stearate, sodium benzoate, sodium acetate, sodium chloride and the like. Disintegrators include, without limitation, starch, methyl cellulose, agar, bentonite, xanthan gum and the like. Solid oral preparations can also be coated with substances such as sugars or be enteric-coated so as to modulate major site of absorption. For parenteral administration, the carrier will usually consist of sterile water and other ingredients can be added to increase solubility or preservation. Injectable suspensions or solutions can also be prepared utilizing aqueous carriers along with appropriate additives. The pharmaceutical compositions herein will contain, per dosage unit, e.g., tablet, capsule, powder, injection, teaspoonful and the like, an amount of the active ingredient necessary to deliver an effective dose as described herein.

The compositions comprising a compound of Formula (I) or a pharmaceutically acceptable salt thereof can be formulated in a unit dosage form, each dosage containing from about 5 to about 1,000 mg (1 g), more usually about 100 mg to about 500 mg, of the active ingredient. The term “unit dosage form” refers to physically discrete units suitable as unitary dosages for human subjects and other subjects, each unit containing a predetermined quantity of active material (i.e., a compound of Formula (I) or a pharmaceutically acceptable salt thereof) calculated to produce the desired therapeutic effect, in association with a suitable pharmaceutical excipient.

In some embodiments, the compositions provided herein contain from about 5 mg to about 50 mg of the active ingredient. One having ordinary skill in the art will appreciate that this embodies compounds or compositions containing about 5 mg to about 10 mg, about 10 mg to about 15 mg, about 15 mg to about 20 mg, about 20 mg to about 25 mg, about 25 mg to about 30 mg, about 30 mg to about 35 mg, about 35 mg to about 40 mg, about 40 mg to about 45 mg, or about 45 mg to about 50 mg of the active ingredient.

In some embodiments, the compositions provided herein contain from about 50 mg to about 500 mg of the active ingredient. One having ordinary skill in the art will appreciate that this embodies compounds or compositions containing about 50 mg to about 100 mg, about 100 mg to about 150 mg, about 150 mg to about 200 mg, about 200 mg to about 250 mg, about 250 mg to about 300 mg, about 350 mg to about 400 mg, or about 450 mg to about 500 mg of the active ingredient. In some embodiments, the compositions provided herein contain about 10 mg, about 20 mg, about 80 mg, or about 160 mg of the active ingredient.

In some embodiments, the compositions provided herein contain from about 500 mg to about 1,000 mg of the active ingredient. One having ordinary skill in the art will appreciate that this embodies compounds or compositions containing about 500 mg to about 550 mg, about 550 mg to about 600 mg, about 600 mg to about 650 mg, about 650 mg to about 700 mg, about 700 mg to about 750 mg, about 750 mg to about 800 mg, about 800 mg to about 850 mg, about 850 mg to about 900 mg, about 900 mg to about 950 mg, or about 950 mg to about 1,000 mg of the active ingredient.

The daily dosage of the compound of Formula (I) or a pharmaceutically acceptable salt thereof can be varied over a wide range from 1.0 to 10,000 mg per adult human per day, or higher, or any range therein. For oral administration, the compositions are preferably provided in the form of tablets containing, 0.01, 0.05, 0.1, 0.5, 1.0, 2.5, 5.0, 10.0, 15.0, 25.0, 50.0, 100, 150, 160, 200, 250 and 500 milligrams of the active ingredient for the symptomatic adjustment of the dosage to the subject to be treated. An effective amount of the drug is ordinarily supplied at a dosage level of from about 0.1 mg/kg to about 1000 mg/kg of body weight per day, or any range therein.

Preferably, the range is from about 0.5 to about 500 mg/kg of body weight per day, or any range therein. More preferably, from about 1.0 to about 250 mg/kg of body weight per day, or any range therein. More preferably, from about 0.1 to about 100 mg/kg of body weight per day, or any range therein. In an example, the range can be from about 0.1 to about 50.0 mg/kg of body weight per day, or any amount or range therein. In another example, the range can be from about 0.1 to about 15.0 mg/kg of body weight per day, or any range therein. In yet another example, the range can be from about 0.5 to about 7.5 mg/kg of body weight per day, or any amount to range therein.

Pharmaceutical compositions containing a compound of Formula (I) or a pharmaceutically acceptable salt thereof can be administered on a regimen of 1 to 4 times per day or in a single daily dose.

The active compound may be effective over a wide dosage range and is generally administered in a pharmaceutically effective amount. Optimal dosages to be administered can be readily determined by those skilled in the art. It will be understood, therefore, that the amount of the compound actually administered will usually be determined by a physician, and will vary according to the relevant circumstances, including the mode of administration, the actual compound administered, the strength of the preparation, the condition to be treated, and the advancement of the disease condition. In addition, factors associated with the particular subject being treated, including subject response, age, weight, diet, time of administration and severity of the subject's symptoms, will result in the need to adjust dosages.

In some embodiments, the compounds provided herein can be administered in an amount ranging from about 1 mg/kg to about 100 mg/kg. In some embodiments, the compound provided herein can be administered in an amount of about 1 mg/kg to about 20 mg/kg, about 5 mg/kg to about 50 mg/kg, about 10 mg/kg to about 40 mg/kg, about 15 mg/kg to about 45 mg/kg, about 20 mg/kg to about 60 mg/kg, or about 40 mg/kg to about 70 mg/kg. For example, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, or about 100 mg/kg.

One skilled in the art will recognize that both in vivo and in vitro trials using suitable, known and generally accepted cell and/or animal models are predictive of the ability of a test compound to treat or prevent a given disorder.

One skilled in the art will further recognize that human clinical trials including first-in-human, dose ranging and efficacy trials, in healthy subjects and/or those suffering from a given disorder, can be completed according to methods well known in the clinical and medical arts.

Provided herein are pharmaceutical kits useful, for example, in the treatment of MALT1-associated diseases or disorders, such as cancer, which include one or more containers containing a pharmaceutical composition comprising an effective amount of a compound provided herein.

Such kits can further include, if desired, one or more of various conventional pharmaceutical kit components, such as, for example, containers with one or more pharmaceutically acceptable carriers, additional containers, etc., as will be readily apparent to those skilled in the art.

Instructions, either as inserts or as labels, indicating quantities of the components to be administered, guidelines for administration, and/or guidelines for mixing the components, can also be included in the kit.

### Additional Embodiments

Embodiment 1. A compound of Formula (I), or a pharmaceutically acceptable salt thereof:

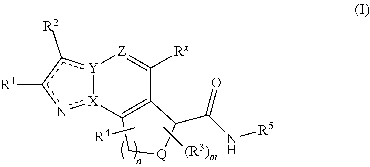

wherein:
each [symbol] is a single or double bond;
Q is —CH₂—, O, or NH;
X is N or C;
Y is N or C;
Z is N or CR⁶;
wherein when one of X and Y is N, the other of X and Y is C;
n is 1, 2, or 3;
R^{X} is hydrogen or halogen;
R¹ is hydrogen, halogen, cyano, hydroxyl, C1-C3 alkoxy, C1-C3 haloalkoxy, C1-C3 haloalkyl, —NR^{A}R^{B}, or C1-C3 alkyl optionally substituted with 1-3 substituents selected from hydroxyl and C1-C3 alkoxy;
R² is hydrogen, halogen, amino, or C1-C3 alkyl;
each R³ is independently deuterium, halogen, hydroxyl, C3-C6 cycloalkyl, C1-C3 alkyl, C1-C3 alkoxy, C1-C3 haloalkoxy, or C1-C3 haloalkyl;
m is 0, 1, 2, or 3;
R⁴ is 5-10 membered heteroaryl optionally substituted with one to three substituents each independently selected from R⁷;
R⁵ is phenyl or 5-9 membered heteroaryl, wherein each R⁵ group is optionally substituted with 1-3 substituents independently selected from R¹;
R⁶ is hydrogen, halogen, cyano, hydroxyl, C1-C3 alkoxy, C1-C3 haloalkoxy, C1-C3 haloalkyl, —NR^{c}R^{D}, and C1-C3 alkyl;
each R⁷ is independently selected from the group consisting of halogen, cyano, hydroxyl, C1-C3 alkoxy, C1-C3 haloalkoxy, C1-C3 haloalkyl, oxetanyl, —NR^{C}R^{D}, C1-C3 alkyl, and C3-C7 cycloalkyl;
each R⁸ is independently selected from halogen; cyano; amino; —N═(S═O)(C1-C3 alkyl)₂; —S(═O)_{F}(C1-C3 alkyl); —(C═O)NR^{F}R^{F}; C1-C3 alkoxy; C1-C3 haloalkyl optionally substituted with hydroxyl; C1-C3 haloalkoxy; 5-6 membered heteroaryl optionally substituted with halogen, cyano, hydroxyl, C1-C3 alkoxy, C1-C3 haloalkoxy, amino, C1-C3 haloalkyl, 4-6 membered heterocyclyl, or C1-C3 alkyl optionally substituted with hydroxyl or —NR^{E}R^{F}; C1-C4 alkyl optionally substituted with hydroxyl, —NR^{E}R^{F}, or C1-C3 alkoxy; 3-8 membered heterocyclyl; and C3-C6 cycloalkoxy;
p is 1 or 2; and
R^{A}, R^{B}, R^{C}, R^{D}, R^{E}, and R^{F}, are independently hydrogen, C1-C3 alkyl, C3-C6 cycloalkyl, or R^{A} and R^{B}, or R^{C} and R^{D}, or R^{E} and R^{F}, together with the nitrogen atom to which they are attached come together to form a 4-6 membered heterocyclyl optionally substituted with 1-2 halogens.

Embodiment 2. The compound of embodiment 1, wherein X is N and Y is C.

Embodiment 3. The compound of embodiment 1 or 2, wherein Z is N.

Embodiment 4. The compound of any one of embodiments 1-3, wherein R^{X} is hydrogen.

Embodiment 5. The compound of any one of embodiments 1-4, wherein Q is —CH₂—.

Embodiment 6. The compound of any one of embodiments 1-5, wherein R¹ is hydrogen.

Embodiment 7. The compound of any one of embodiments 1-5, wherein R¹ is halogen.

Embodiment 8. The compound of any one of embodiments 1-7, wherein R² is hydrogen.

Embodiment 9. The compound of any one of embodiments 1-7, wherein R² is halogen.

Embodiment 10. The compound of any one of embodiments 1-9, wherein n is 1.

Embodiment 11. The compound of any one of embodiments 1-10, wherein m is 1.

Embodiment 12. The compound of any one of embodiments 1-11, wherein each R³ is independently deuterium, halogen, hydroxyl, C3-C6 cycloalkyl, C1-C3 alkyl, C1-C3 haloalkyl, C1-C3 alkoxy, or C1-C3 haloalkoxy.

Embodiment 13. The compound of any one of embodiments 1-12, wherein m is 1 and R³ is methyl or trifluoromethyl.

Embodiment 14. The compound of any one of embodiments 1-13, wherein R⁴ is a 5 or 6 membered heteroaryl optionally substituted with one to three substituents each independently selected from R⁷.

Embodiment 15. The compound of any one of embodiments 1-13, wherein R⁴ is a 6 membered heteroaryl optionally substituted with one to three substituents each independently selected from R⁷.

Embodiment 16. The compound of any one of embodiments 1-13, wherein R⁴ is a 5 membered heteroaryl optionally substituted with one to three substituents each independently selected from R⁷.

Embodiment 17. The compound of any one of embodiments 1-13, wherein R⁴ is 4H-pyrazolyl, 3H-pyrazolyl, or thiazoyl optionally substituted with one to three substituents each independently selected from R⁷.

Embodiment 18. The compound of any one of embodiments 1-13, wherein R⁴ is unsubstituted.

Embodiment 19. The compound of any one of embodiments 1-13, wherein each R⁷ is independently selected from the group consisting of C1-C3 haloalkyl, C1-C3 alkyl, and cycloalkyl.

Embodiment 20. The compound of any one of embodiments 1-19, wherein R⁵ is 5-9 membered heteroaryl optionally substituted with 1-3 independently selected R⁸.

Embodiment 21. The compound of any one of embodiments 1-19, wherein R⁵ is 5-6 membered heteroaryl substituted with 1-3 independently selected R⁸.

Embodiment 22. The compound of any one of embodiments 1-21, wherein R⁵ is 3-pyridyl or 4-pyridyl optionally substituted with 1-3 independently selected R⁸.

Embodiment 23. The compound of any one of embodiments 1-21, wherein at least one of R⁸ is halogen.

Embodiment 24. The compound of any one of embodiments 1-21, wherein at least one of R⁸ is 5-6 membered heteroaryl optionally substituted with halogen, cyano, hydroxyl, C1-C3 alkoxy, C1-C3 haloalkoxy, C1-C3 alkyl optionally substituted with hydroxyl or —NR^{E}R^{F}, amino, or C1-C3 haloalkyl.

Embodiment 25. The compound of any one of embodiments 1-21, wherein at least one of R⁸ is 5 membered heteroaryl optionally substituted with halogen, cyano, hydroxyl, C1-C3 alkoxy, C1-C3 haloalkoxy, C1-C3 alkyl optionally substituted with hydroxyl or —NR^{E}R^{F}, amino, or C1-C3 haloalkyl.

Embodiment 26. A compound according to anyone of embodiments 1-25, which is a compound according to formula (Ib), or a pharmaceutically acceptable salt thereof

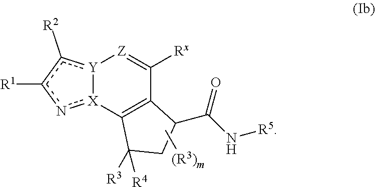

Embodiment 27. A pharmaceutical composition comprising a compound according to any one of embodiments 1-26, or a pharmaceutically acceptable salt thereof, in combination with one or more pharmaceutically acceptable excipients.

Embodiment 28. A method for treating cancer in a subject in need thereof, comprising administering to the subject an effective amount of a compound of any one of embodiments 1-26, or a pharmaceutically acceptable salt thereof, or a pharmaceutical composition of embodiment 27.

Embodiment 29. The method of embodiment 28 where the cancer is a MALT1-associated cancer.

Embodiment 30. The method of embodiment 28 or 29 wherein the cancer is lymphoma.

Embodiment 31. The method of embodiment 30 wherein the lymphoma is non-Hodgkin lymphoma, DLBCL, refractory DLBCL, (ABC) subtype of DLBCL, mantle cell lymphoma (MCL), or marginal zone lymphoma (MZL).

Embodiment 32. The method of embodiment 28 or 29 wherein the cancer is leukemia.

Embodiment 33. The method of embodiment 32 where in the leukemia is chronic lymphocytic leukemia (CLL).

Embodiment 34. The method of embodiment 28 or 29 wherein the cancer is a solid tumor.

Embodiment 35. A method of treating an autoimmune disorder in a subject in need thereof, comprising administering to the subject an effective amount of a compound of any one of embodiments 1-26, or a pharmaceutically acceptable salt thereof, or a pharmaceutical composition of embodiment 27.

Embodiment 36. The method according to embodiment 35 wherein the autoimmune disorder is chronic graph versus host disease.

Embodiment 37. The method of any one of embodiments 30-36, further comprising administering an additional therapy or therapeutic agent to the subject.

### EXAMPLES

### Materials and Methods

The compounds provided herein, including salts thereof, can be prepared using known organic synthesis techniques and can be synthesized according to any of numerous possible synthetic routes.

The reactions for preparing the compounds provided herein can be carried out in suitable solvents which can be readily selected by one of skill in the art of organic synthesis. Suitable solvents can be substantially non-reactive with the starting materials (reactants), the intermediates, or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

Preparation of the compounds provided herein can involve the protection and deprotection of various chemical groups. The need for protection and deprotection, and the selection of appropriate protecting groups, can be readily determined by one skilled in the art. The chemistry of protecting groups can be found, for example, in Protecting Group Chemistry, 1^{st} Ed., Oxford University Press, 2000; March's Advanced Organic Chemistry: Reactions, Mechanisms, and Structure, 5^{th} Ed., Wiley-Interscience Publication, 2001; and Peturssion, S. et al., “Protecting Groups in Carbohydrate Chemistry,” J. Chem. Educ., 74(11), 1297 (1997).

Reactions sensitive to moisture or air were performed under nitrogen or argon using anhydrous solvents and reagents. The progress of reactions was determined by either analytical thin layer chromatography (TLC) usually performed with Sanpont precoated TLC plates, silica gel GF-254, layer thickness 0.25 mm or liquid chromatography-mass spectrometry (LC-MS).

Typically, the analytical LC-MS system used consisted of Shimadzu LCMS-2020 with electrospray ionization in positive ion detection mode with 20ADXR pump, SIL-20ACXR autosampler, CTO-20AC column oven, M20A PDA Detector and LCMS 2020 MS detector. The column was usually HALO a C18 30*5.0 mm, 2.7 μm. The mobile phase A is water containing 0.05% TFA and mobile phase B is acetonitrile containing 0.05% TFA. The gradient is from 5% mobile phase B to 100% in 2.0 min, hold 0.7 min, then reverting to 5% mobile phase B over 0.05 min and maintained for 0.25 min. The Column Oven (CTO-20AC) was operated at a temperature of 40.0° C. The flow rate was 1.5 mL/min, and the injection volume was 1 μl. PDA (SPD-M20A) detection was in the range 190-400 nm. The MS detector, which was configured with electrospray ionization as ionizable source; Acquisition mode: Scan; Nebulizing Gas Flow: 1.5 L/min; Drying Gas Flow: 15 L/min; Detector Voltage: Tuning Voltage ±0.2 kv; DL Temperature: 250° C.; Heat Block Temperature: 250° C.; Scan Range: 90.00-900.00 m/z. ELSD (Alltech 3300) detector Parameters: Drift Tube Temperature: 60±5° C.; N₂ Flow-Rate: 1.8±0.2 L/min. Mobile phase gradients were optimized for the individual compounds.

The GC-MS system was usually performed with Shimadzu GCMS-QP2010 Ultra with FID and MS Detector. The MS detector of acquisition mode: Start Time: 2.00 min; End Time: 9.00 min; ACQ Mode: Scan; Event Time: 0.30 sec; Scan Speed: 2000; Start m/z: 50.00; End m/z: 550.00; Ion Source temperature: 200.00° C.; Interface temperature: 250.00° C.; Solvent Cut Time: 2.00 min.

Preparative HPLC purifications were usually performed with Waters Auto purification system (2545-2767) with a 2489 UV detector. The column was Waters C18, 19×150 mm, 5 μm. The mobile phases consisted of mixtures of acetonitrile (5-95%) in water containing 0.1% FA. Flow rates were maintained at 25 mL/min, the injection volume was 1200 μL, and the UV detector used two channels 254 nm and 220 nm. Mobile phase gradients were optimized for the individual compounds.

Chiral analytical chromatography was performed on one of Chiralpak AS, AD, Chiralcel OD, OJ Chiralpak IA, IB, IC, ID, IE, IF, IG, IH columns (Daicel Chemical Industries, Ltd.); (R,R)-Whelk-01, (S,S)-Whelk-01 columns (Regis technologies, Inc.); CHIRAL Cellulose-SB, SC, SA columns (YMC Co., Ltd.) at different column sizes (50×4.6 mm, 100×4.6 mm, 150×4.6 mm, 250×4.6 mm, 50×3.0 mm, 100×3.0 mm) with noted percentage of either ethanol in hexane (% Et/Hex) or isopropanol in hexane (% IPA/Hex) as isocratic solvent systems.

Reactions performed using microwave irradiation were normally carried out using an Initiator manufactured by Biotage. Concentration of solutions was carried out on a rotary evaporator under reduced pressure. Flash column chromatography was usually performed using a Biotage Flash Chromatography apparatus (Dyax Corp.) on silica gel (40-60 μM, 60 Å pore size) in pre-packed cartridges of the size noted. ¹H NMR spectra were acquired at 400 MHz spectrometers in DMSO-d₆ solutions unless otherwise noted. Chemical shifts were reported in parts per million (ppm). Tetramethylsilane (TMS) was used as internal reference in DMSO-d₆ solutions, and residual CH₃OH peak or TMS was used as internal reference in CD₃OD solutions. Coupling constants (J) were reported in hertz (Hz). Chiral analytical chromatography was performed on one of Chiralpak AS, Chiralpak AD, Chiralcel OD, Chiralcel IA, or Chiralcel OJ columns (250×4.6 mm) (Daicel Chemical Industries, Ltd.) with noted percentage of either ethanol in hexane (% Et/Hex) or isopropanol in heptane (% IPA/Hep) as isocratic solvent systems. Chiral preparative chromatography was conducted on one of Chiralpak AS, AD, Chiralcel OD, OJ, Chiralpak IA, IB, IC, ID, IE, IF, IG, IH columns (Daicel Chemical Industries, Ltd.); (R,R)-Whelk-01, (S,S)-Whelk-01 columns (Regis technologies, Inc.); CHIRAL Cellulose-SB, SC, SA columns (YMC Co., Ltd.) at different column size (250×20 mm, 250×30 mm, 250×50 mm) with desired isocratic solvent systems identified on chiral analytical chromatography.

Abbreviations used herein include: —C(O)CH₃ (Ac); acetic acid (AcOH); —OC(O)CH₃ (OAc); aqueous (aq); Cbz (benzyloxycarbonyl); N,N-diisopropylethylamine (DIEA); N; N-dimethylformamide (DMF); 1-Ethyl-3-(3-dimethylaminopropyl)carbodiimide (EDCI); EtOAc (EtOAc); diethyl ether (ether or Et₂O); PE (PE); gram(s) (g); hour(s) (h or hr); 2-propanol (IPA); mass spectrum (ms or MS); microliter(s) (VL); milligram(s) (mg); milliliter(s) (mL); millimole (mmol); minute(s) (min); methyl t-butylether (MTBE); (benzotriazol-1-yloxy)tripyrrolidino-phosphonium hexafluorophosphate (PyBOP); retention time (Rt); rt (rt or RT); saturated aq sodium chloride solution (brine); trifluoroacetic acid (TFA); tetrahydrofuran (THF); flash chromatography (FC); liquid chromatography (LC); liquid chromatography-mass spectrometry (LCMS or LC-MS); supercritical fluid chromatography (SFC); t-butyloxycarbonyl (Boc or BOC); Diethylaminosulfur trifluoride (DAST); DCM (DCM); dimethylacetamide (DMA; DMAC); dimethylsulfoxide (DMSO); 1,3-Bis(diphenylphosphino)propane (DPPP); acetic acid (HOAc); 3-chloroperoxybenzoic acid (m-CPBA); methyl (Me); methanol (MeOH); N,N,N′,N′-tetramethylchloroformamidinium hexafluorophosphate (TCFH); N-methylimidazole (NMI); N-bromosuccinamide (NBS); thin layer chromatography (TLC).

The following are representative procedures for the preparation of the compounds used in the following Examples, or which can be substituted for the compounds used in the following Examples which may not be commercially available.

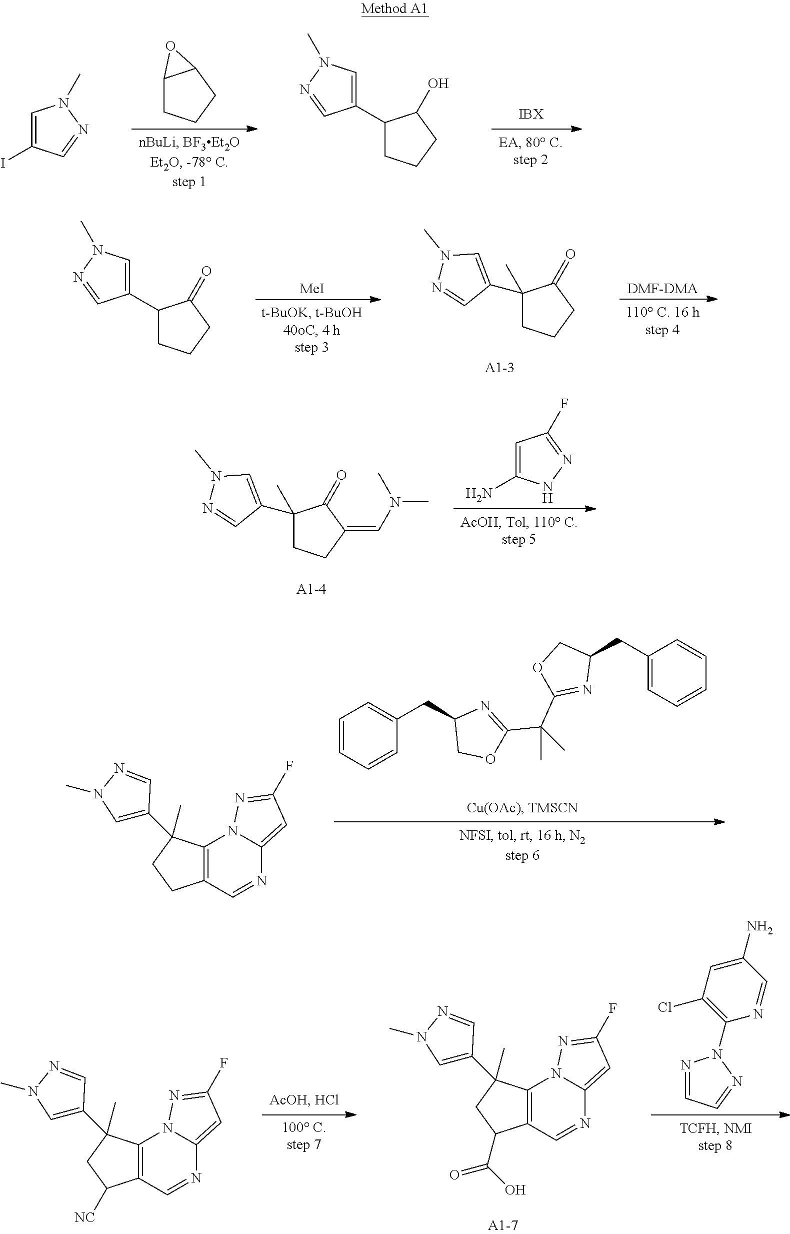

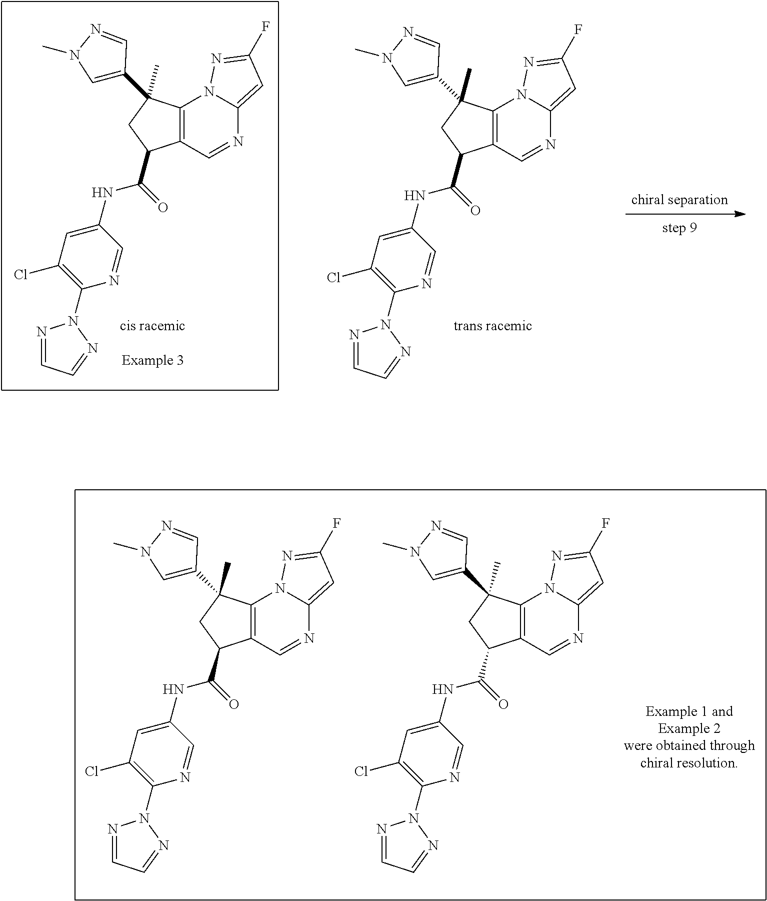 [SMILES: Cn1cc([C@]2(C)C[C@H](C(=O)Nc3cnc(-n4nccn4)c(Cl)c3)c3cnc4cc(F)nn4c32)cn1 ; Cn1cc([C@@]2(C)C[C@H](C(=O)Nc3cnc(-n4nccn4)c(Cl)c3)c3cnc4cc(F)nn4c32)cn1 ; Cn1cc([C@@]2(C)C[C@H](C(=O)Nc3cnc(-n4nccn4)c(Cl)c3)c3cnc4cc(F)nn4c32)cn1 ; Cn1cc([C@]2(C)C[C@@H](C(=O)Nc3cnc(-n4nccn4)c(Cl)c3)c3cnc4cc(F)nn4c32)cn1]

### Examples 1, 2: (6S,8R)—N-(5-chloro-6-(2H-1,2,3-triazol-2-yl)pyridin-3-yl)-2-fluoro-8-methyl-8-(1-methyl-1H-pyrazol-4-yl)-7,8-dihydro-6H-cyclopenta[e]pyrazolo[1,5-a]pyrimidine-6-carboxamide; (6R,8S)—N-(5-chloro-6-(2H-1,2,3-triazol-2-yl)pyridin-3-yl)-2-fluoro-8-methyl-8-(1-methyl-1H-pyrazol-4-yl)-7,8-dihydro-6H-cyclopenta[e]pyrazolo[1,5-a]pyrimidine-6-carboxamide

### Example 3: (cis)-N-(5-chloro-6-(2H-1,2,3-triazol-2-yl)pyridin-3-yl)-2-fluoro-8-methyl-8-(1-methyl-1H-pyrazol-4-yl)-7,8-dihydro-6H-cyclopenta[e]pyrazolo[1,5-a]pyrimidine-6-carboxamide

The relative and absolute stereochemistry of each example was not determined.

### Step 1: 2-(1-methyl-1H-pyrazol-4-yl)cyclopentan-1-ol

To a solution of 4-iodo-1-methyl-pyrazole (48 g, 231.8 mmol) in Et₂O (2 L) was added n-Butyllithium (14.8 g, 231.8 mmol, 2.5 M in hexane) at −78° C. The reaction mixture was stirred at −78° C. for 30 min under nitrogen. To the above mixture was added 6-oxabicyclo[3.1.0]hexane (15 g, 178.3 mmol) and Boron trifluoride diethyl etherate (32.9 g, 231.8 mmol). The reaction mixture was stirred at −78° C. for 30 min under nitrogen. The reaction mixture was quenched with saturated aqueous ammonium chloride (500 mL). The resulting mixture was extracted with ethyl acetate (3×500 mL). The combined organic layers were dried over anhydrous sodium sulfate and concentrated under reduced pressure. The residue was applied onto a silica gel column and eluted with DCM/MeOH (10:1) to give the title compound (8 g, 21% yield) as a yellow oil. 1H NMR (400 MHz, Chloroform-d) δ 7.37 (s, 1H), 7.24 (s, 1H), 3.99-4.07 (m, 1H), 3.87 (s, 3H), 2.79-2.88 (m, 1H), 2.00-2.21 (m, 2H), 1.56-1.92 (m, 4H). LC-MS: m/z 167 [M+H]⁺.

### Step 2: 2-(1-methyl-1H-pyrazol-4-yl)cyclopentan-1-one

To a stirred solution of 2-(1-methyl-1H-pyrazol-4-yl)cyclopentan-1-ol (5 g, 30 mmol) in ethyl acetate (250 mL) was added TBX (27.4 g, 45.1 mmol, 46% purity). The resulting mixture was stirred at 80° C. for 16 h. The mixture was allowed to cool to 25° C. The reaction was quenched with saturated aqueous NaHSO₃ (200 mL). The resulting mixture was extracted with ethyl acetate (3×200 mL). The combined organic layers were washed with saturated aqueous sodium bicarbonate solution (200 mL), dried over anhydrous sodium sulfate and concentrated under reduced pressure. The residue was purified by prep-HPLC to give the title compound (1.6 g, 32% yield) as a yellow oil. LC-MS: m/z 165 [M+H]⁺.

### Step 3: 2-methyl-2-(1-methyl-1H-pyrazol-4-yl)cyclopentan-1-one (A1-3)

To a stirred solution of 2-(1-methyl-1H-pyrazol-4-yl)cyclopentan-1-one (3.9 g, 23.7 mmol) in t-BuOH (60 mL) was added potassium 2-methylpropan-2-olate (2.9 g, 26.1 mmol) in portions at 25° C. The resulting mixture was stirred at 40° C. for 0.5 h. Iodomethane (3.4 g, 23.7 mmol) was added dropwise at 40° C. and the resulting mixture was stirred at 40° C. for 2 h. The reaction was quenched with water (200 mL), and the mixture was extracted with ethyl acetate (3×200 mL). The combined organic layers were dried over anhydrous sodium sulfate and concentrated under reduced pressure. The residue was applied onto a silica gel column and eluted with THF/PE (1:1) to give the title compound (A1-3, 3.9 g, 82% yield) as a yellow oil. LC-MS: m/z 179 [M+H]⁺.

### Step 4: 5-((dimethylamino)methylene)-2-methyl-2-(1-methyl-1H-pyrazol-4-yl)cyclopentan-1-one (A1-4)

A solution of 2-methyl-2-(1-methyl-1H-pyrazol-4-yl)cyclopentan-1-one (5.5 g, 30.8 mmol) in 1,1-dimethoxy-N,N-dimethylmethanamine (70 mL) was stirred at 100° C. for 24 h. The mixture was allowed to cool to 25° C. and then concentrated under reduced pressure to give the crude title compound (A1-4, 5.5 g) as a red oil. LC-MS: m/z 234 [M+H]⁺.

### Step 5: 2-fluoro-8-methyl-8-(1-methyl-1H-pyrazol-4-yl)-7,8-dihydro-6H-cyclopenta[e]pyrazolo[1,5-a]pyrimidine

To a stirred solution of 5-((dimethylamino)methylene)-2-methyl-2-(1-methyl-1H-pyrazol-4-yl)cyclopentan-1-one (2 g, 8.5 mmol) in toluene (20 mL) were added 3-fluoro-1H-pyrazol-5-amine (1 g, 10.3 mmol) and AcOH (2 mL). The resulting mixture was stirred at 110° C. for 16 h. The mixture was allowed to cool to 25° C. The reaction mixture was concentrated under reduced pressure. The residue was diluted with water (100 mL). The resulting solution was extracted with ethyl acetate (3×100 mL). The combined organic layers were dried over anhydrous sodium sulfate and concentrated under reduced pressure. The residue was applied onto a silica gel column and eluted with EtOAc/PE (1:1) to give the title compound (2 g, 60% yield) as a light-yellow solid. 1H NMR (300 MHz, DMSO-d₆) δ 8.60 (s, 1H), 7.63 (s, 1H), 7.39 (s, 1H), 6.47 (d, J=5.1 Hz, 1H), 3.75 (s, 3H), 2.97-3.21 (m, 2H), 2.39-2.57 (m, 1H), 2.28-2.32 (m, 1H), 1.88 (s, 3H). LC-MS: m/z 272 [M+H]⁺.

### Step 6: 2-fluoro-8-methyl-8-(1-methyl-1H-pyrazol-4-yl)-7,8-dihydro-6H-cyclopenta[e]pyrazolo[1,5-a]pyrimidine-6-carbonitrile

To a stirred solution of 2-fluoro-8-methyl-8-(1-methyl-1H-pyrazol-4-yl)-7,8-dihydro-6H-cyclopenta[e]pyrazolo[1,5-a]pyrimidine (2 g, 7.4 mmol) in toluene (80 mL) were added (4R)-4-benzyl-2-[1-[(4R)-4-benzyl-4,5-dihydrooxazol-2-yl]-1-methyl-ethyl]-4,5-dihydrooxazole (641 mg, 1.8 mmol), acetoxycopper (181 mg, 1.5 mmol), N-Fluorobenzenesulfonimide (3.4 g, 11.1 mmol) and TMSCN (3.7 g, 36.9 mmol). The reaction was stirred at 25° C. for 16 h under nitrogen. The solvent was removed under reduced pressure and the residue was applied on a silica gel column and eluted with EtOAc/PE (1:1) to get the crude product. The crude product was further purified by prep-HPLC to give the title compound (100 mg, 4% yield) as a yellow oil. LC-MS (ES, m/z): 297[M+H]⁺.

### Step 7: 2-fluoro-8-methyl-8-(1-methyl-1H-pyrazol-4-yl)-7,8-dihydro-6H-cyclopenta[e]pyrazolo[1,5-a]pyrimidine-6-carboxylic acid (A1-7)

A solution of 2-fluoro-8-methyl-8-(1-methyl-1H-pyrazol-4-yl)-7,8-dihydro-6H-cyclopenta[e]pyrazolo[1,5-a]pyrimidine-6-carbonitrile (130 mg, 0.44 mmol) in AcOH (2 mL) and HCl (12M, 2 mL) was stirred at 100° C. for 2 h. The mixture was allowed to cool to 25° C. The mixture was concentrated under reduced pressure and the residue was diluted with water (50 ml). The resulting mixture was extracted with ethyl acetate (3×50 mL). The combined organic layers were dried over anhydrous sodium sulfate and concentrated under reduced pressure to give the title compound (A1-7, 130 mg, 90% yield) as a yellow oil. LC-MS: m/z 316 [M+H]⁺.

### Step 8: Examples 3 and trans-N-(5-chloro-6-(2H-1,2,3-triazol-2-yl)pyridin-3-yl)-2-fluoro-8-methyl-8-(1-methyl-1H-pyrazol-4-yl)-7,8-dihydro-6H-cyclopenta[e]pyrazolo[1,5-a]pyrimidine-6-carboxamide

To a stirred solution of 2-fluoro-8-methyl-8-(1-methyl-1H-pyrazol-4-yl)-7,8-dihydro-6H-cyclopenta[e]pyrazolo[1,5-a]pyrimidine-6-carboxylic acid (165 mg, 523 μmol) and 5-chloro-6-(2H-1,2,3-triazol-2-yl)pyridin-3-amine (123 mg, 628 μmol) in ACN (10 mL) were added TCFH (587 mg, 2.1 mmol) and NMI (215 mg, 2.6 mmol). The reaction mixture was stirred at 25° C. for 1 h. The reaction mixture was concentrated under reduced pressure. The residue was diluted with water (100 mL). The resulting mixture was extracted with ethyl acetate (3×100 mL). The combined organic layers were dried over anhydrous sodium sulfate and concentrated under reduced pressure. The residue was applied on a silica gel column and eluted with DCM/MeOH (10:1) to give the crude product. The crude product was further purified by prep-HPLC to afford Example 3 (cis-racemic mixture, 80 mg. 29% yield,) as a white solid and a racemic mixture of trans-N-(5-chloro-6-(2H-1,2,3-triazol-2-yl)pyridin-3-yl)-2-fluoro-8-methyl-8-(1-methyl-1H-pyrazol-4-yl)-7,8-dihydro-6H-cyclopenta[e]pyrazolo[1,5-a]pyrimidine-6-carboxamide (120 mg, 44% yield) as a white solid.

Example 3: ¹H NMR (300 MHz, DMSO-d₆) δ 11.10 (s, 1H), 8.73 (d, J=2.4 Hz, 1H), 8.71 (s, 1H), 8.57 (d, J=2.4 Hz, 1H), 8.18 (s, 2H), 7.62 (s, 1H), 7.35 (s, 1H), 6.55 (d, J=5.1 Hz, 1H), 4.51-4.56 (m, 1H), 3.76 (s, 3H), 2.68-2.84 (m, 2H), 1.88 (s, 3H). LC-MS: m/z 493 [M+H]⁺.

### Step 9: Separation of Enantiomers to Obtain Example 1 and Example 2

120 mg of trans-N-(5-chloro-6-(2H-1,2,3-triazol-2-yl)pyridin-3-yl)-2-fluoro-8-methyl-8-(1-methyl-1H-pyrazol-4-yl)-7,8-dihydro-6H-cyclopenta[e]pyrazolo[1,5-a]pyrimidine-6-carboxamide were submitted to chiral HPLC purification (Column: CHIRAL ART Cellulose-SA, 2*25 cm, 5 μm; Mobile Phase A: Hex (0.1% FA)-HPLC, Mobile Phase B: IPA-HPLC; Flow rate: 20 mL/min; isocratic: 50% B in 25 min; Wave Length: 220/254 nm; RT1 (min): 10.008; RT2 (min): 19.257; Sample Solvent: EtOH-HPLC; Injection Volume: 1.5 mL; Number Of Runs: 3). The first eluting isomer was concentrated and lyophilized to afford Example 1 (40.7 mg, 33% yield) as a white solid. The second eluting isomer was concentrated and lyophilized to afford Example 2 (33.9 mg, 28% yield) as a white solid.

Example 1: ¹H NMR (300 MHz, DMSO-d₆) δ 11.07 (s, 1H), 8.73 (d, J=2.4 Hz, 1H), 8.67 (s, 1H), 8.61 (d, J=2.4 Hz, 1H), 8.18 (s, 2H), 7.65 (s, 1H), 7.43 (s, 1H), 6.58 (d, J=5.1 Hz, 1H), 4.54 (t, J=8.4 Hz, 1H), 3.76 (s, 3H), 2.85-2.98 (m, 1H), 2.50-2.56 (m, 1H), 1.97 (s, 3H). LC-MS: m/z 493 [M+H]⁺.

Example 2: ¹H NMR (300 MHz, DMSO-d₆) δ 11.07 (s, 1H), 8.73 (d, J=2.4 Hz, 1H), 8.67 (s, 1H), 8.61 (d, J=2.4 Hz, 1H), 8.18 (s, 2H), 7.65 (s, 1H), 7.43 (s, 1H), 6.58 (d, J=5.1 Hz, 1H), 4.54 (t, J=8.4 Hz, 1H), 3.76 (s, 3H), 2.85-2.98 (m, 1H), 2.50-2.56 (m, 1H), 1.97 (s, 3H). LC-MS: m/z 493 [M+H]⁺.

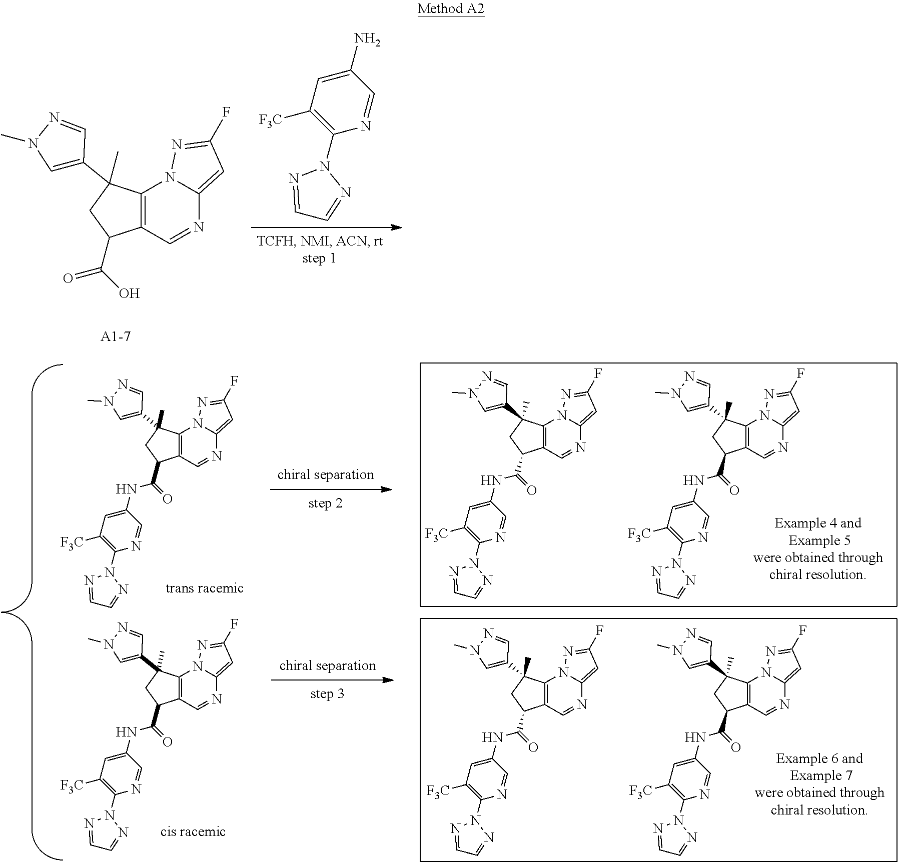

### Examples 4, 5: (6R,8S)—N-(6-(2H-1,2,3-triazol-2-yl)-5-(trifluoromethyl)pyridin-3-yl)-2-fluoro-8-methyl-8-(1-methyl-1H-pyrazol-4-yl)-7,8-dihydro-6H-cyclopenta[e]pyrazolo[1,5-a]pyrimidine-6-carboxamide; (6S,8R)—N-(6-(2H-1,2,3-triazol-2-yl)-5-(trifluoromethyl)pyridin-3-yl)-2-fluoro-8-methyl-8-(1-methyl-1H-pyrazol-4-yl)-7,8-dihydro-6H-cyclopenta[e]pyrazolo[1,5-a]pyrimidine-6-carboxamide

### Examples 6, 7: (6R,8R)—N-(6-(2H-1,2,3-triazol-2-yl)-5-(trifluoromethyl)pyridin-3-yl)-2-fluoro-8-methyl-8-(1-methyl-1H-pyrazol-4-yl)-7,8-dihydro-6H-cyclopenta[e]pyrazolo[1,5-a]pyrimidine-6-carboxamide; (6S,8S)—N-(6-(2H-1,2,3-triazol-2-yl)-5-(trifluoromethyl)pyridin-3-yl)-2-fluoro-8-methyl-8-(1-methyl-1H-pyrazol-4-yl)-7,8-dihydro-6H-cyclopenta[e]pyrazolo[1,5-a]pyrimidine-6-carboxamide

The relative and absolute stereochemistry of each example was not determined.

### Step 1: trans-N-(6-(2H-1,2,3-triazol-2-yl)-5-(trifluoromethyl)pyridin-3-yl)-2-fluoro-8-methyl-8-(1-methyl-1H-pyrazol-4-yl)-7,8-dihydro-6H-cyclopenta[e]pyrazolo[1,5-a]pyrimidine-6-carboxamide and cis-N-(6-(2H-1,2,3-triazol-2-yl)-5-(trifluoromethyl)pyridin-3-yl)-2-fluoro-8-methyl-8-(1-methyl-1H-pyrazol-4-yl)-7,8-dihydro-6H-cyclopenta[e]pyrazolo[1,5-a]pyrimidine-6-carboxamide

In analogy to Method A1 step 8 but using 2-fluoro-8-methyl-8-(1-methyl-1H-pyrazol-4-yl)-7,8-dihydro-6H-cyclopenta[e]pyrazolo[1,5-a]pyrimidine-6-carboxylic acid (A1-7, 128 mg, 405.9 μmol) and 6-(2H-1,2,3-triazol-2-yl)-5-(trifluoromethyl)pyridin-3-amine (93 mg, 405.9 mol), racemic mixtures of the title compounds were obtained: trans isomer (65 mg, 30% yield) as a white solid, LC-MS: m/z 527 [M+H]⁺; cis-isomer (60 mg, 28% yield) as a white solid, LC-MS: m/z 527 [M+H]⁺.

### Step 2: Separation of Enantiomers to Obtain Example 4 and Example 5

65 mg of trans-N-(6-(2H-1,2,3-triazol-2-yl)-5-(trifluoromethyl)pyridin-3-yl)-2-fluoro-8-methyl-8-(1-methyl-1H-pyrazol-4-yl)-7,8-dihydro-6H-cyclopenta[e]pyrazolo[1,5-a]pyrimidine-6-carboxamide were submitted to chiral HPLC purification (Column: CHIRAL ART Amylose-SA, 2*25 cm, 5 μm; Mobile Phase A: Hex (0.1% FA)-HPLC, Mobile Phase B: IPA-HPLC; Flow rate: 20 mL/min; isocratic: 50% B in 20 min; Wave Length: 220/254 nm; RT1 (min): 7.514; RT2 (min): 19.166; Sample Solvent: EtOH-HPLC; Injection Volume: 2 mL; Number Of Runs: 2). The first eluting isomer was concentrated and lyophilized to afford Example 4 (17 mg, 26% yield) as a white solid. The second eluting isomer was concentrated and lyophilized to afford Example 5 (24.6 mg, 37% yield) as a white solid.

Example 4: ¹H NMR (300 MHz, DMSO-d₆) δ: 11.21 (s, 1H), 9.02 (d, J=2.4 Hz, 1H), 8.83 (d, J=2.4 Hz, 1H), 8.69 (s, 1H), 8.20 (s, 2H), 7.65 (s, 1H), 7.42 (s, 1H), 6.58 (d, J=4.8 Hz, 1H), 4.53-4.58 (m, 1H), 3.76 (s, 3H), 2.88-2.99 (m, 1H), 2.51-2.58 (m, 1H), 1.97 (s, 3H). LC-MS: m/z 527 [M+H]⁺.

Example 5: ¹H NMR (300 MHz, DMSO-d₆) δ: 11.21 (s, 1H), 9.02 (d, J=2.1 Hz, 1H), 8.83 (d, J=2.4 Hz, 1H), 8.69 (s, 1H), 8.20 (s, 2H), 7.65 (s, 1H), 7.43 (s, 1H), 6.58 (d, J=4.8 Hz, 1H), 4.53-4.58 (m, 1H), 3.76 (s, 3H), 2.88-2.95 (m, 1H), 2.50-2.58 (m, 1H), 1.97 (s, 3H). LC-MS: m/z 527 [M+H]⁺.

### Step 3: Separation of Enantiomers to Obtain Example 6 and Example 7

60 mg of cis-N-(6-(2H-1,2,3-triazol-2-yl)-5-(trifluoromethyl)pyridin-3-yl)-2-fluoro-8-methyl-8-(1-methyl-1H-pyrazol-4-yl)-7,8-dihydro-6H-cyclopenta[e]pyrazolo[1,5-a]pyrimidine-6-carboxamide were submitted to chiral HPLC purification (Column: CHIRAL ART Amylose-SA, 2*25 cm, 5 m; Mobile Phase A: Hex (0.1% FA)-HPLC, Mobile Phase B: EtOH-HPLC; Flow rate: 20 mL/min; isocratic: 25% B in 13 min; Wave Length: 220/254 nm; RT1 (min): 8.968; RT2 (min): 11.575; Sample Solvent: EtOH-HPLC; Injection Volume: 0.5 mL; Number Of Runs: 8). The first eluting isomer was concentrated and lyophilized to afford Example 6 (23 mg, 38% yield) as a white solid. The second eluting isomer was concentrated and lyophilized to afford Example 7 (24.6 mg, 41% yield) as a white solid.

Example 6: ¹H NMR (300 MHz, DMSO-d₆) δ: 11.20 (s, 1H), 9.03 (d, J=2.1 Hz, 1H), 8.79 (d, J=2.4 Hz, 1H), 8.72 (s, 1H), 8.19 (s, 2H),7.62 (s, 1H), 7.35 (s, 1H), 6.55 (d, J=4.8 Hz, 1H), 4.54-4.57 (m, 1H), 3.74 (s, 3H), 2.69-2.88 (m, 2H), 1.87 (s, 3H). LC-MS: m/z 527 [M+H]⁺.

Example 7: ¹H NMR (300 MHz, DMSO-d₆) δ: 11.21 (s, 1H), 9.03 (d, J=2.4 Hz, 1H), 8.79 (d, J=2.4 Hz, 1H), 8.72 (s, 1H), 8.19 (s, 2H), 7.62 (s, 1H), 7.34 (s, 1H), 6.55 (d, J=5.1 Hz, 1H), 4.54-4.57 (m, 1H), 3.75 (s, 3H), 2.69-2.88 (m, 2H), 1.87 (s, 3H). LC-MS: m/z 527 [M+H]⁺.

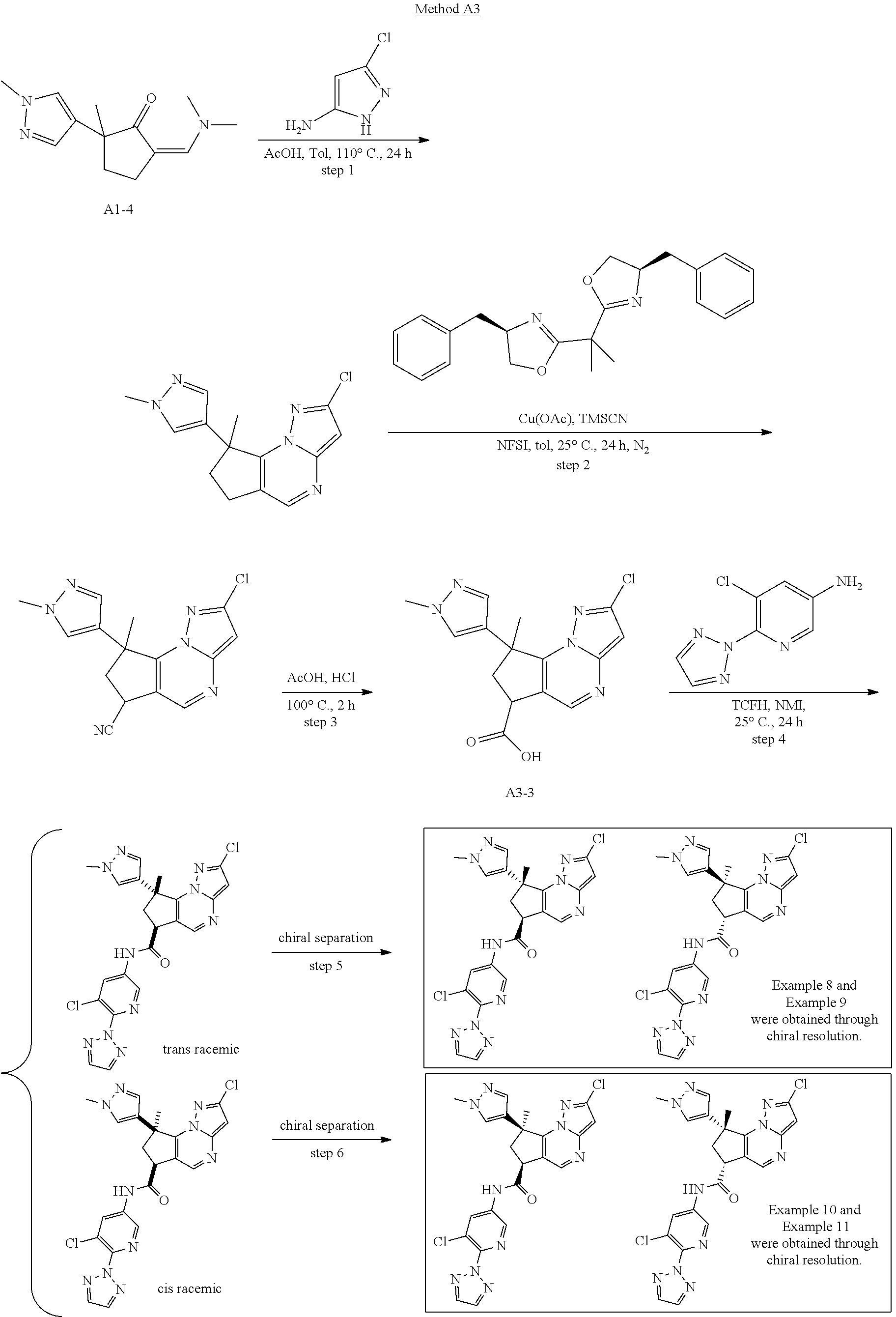

### Examples 8, 9: (6S,8R)-2-chloro-N-(5-chloro-6-(2H-1,2,3-triazol-2-yl)pyridin-3-yl)-8-methyl-8-(1-methyl-1H-pyrazol-4-yl)-7,8-dihydro-6H-cyclopenta[e]pyrazolo[1,5-a]pyrimidine-6-carboxamide; (6R,8S)-2-chloro-N-(5-chloro-6-(2H-1,2,3-triazol-2-yl)pyridin-3-yl)-8-methyl-8-(1-methyl-1H-pyrazol-4-yl)-7,8-dihydro-6H-cyclopenta[e]pyrazolo[1,5-a]pyrimidine-6-carboxamide

### Examples 10, 11: (6S,8S)-2-chloro-N-(5-chloro-6-(2H-1,2,3-triazol-2-yl)pyridin-3-yl)-8-methyl-8-(1-methyl-1H-pyrazol-4-yl)-7,8-dihydro-6H-cyclopenta[e]pyrazolo[1,5-a]pyrimidine-6-carboxamide; (6R,8R)-2-chloro-N-(5-chloro-6-(2H-1,2,3-triazol-2-yl)pyridin-3-yl)-8-methyl-8-(1-methyl-1H-pyrazol-4-yl)-7,8-dihydro-6H-cyclopenta[e]pyrazolo[1,5-a]pyrimidine-6-carboxamide

The relative and absolute stereochemistry of each example was not determined.

### Step 1: 2-chloro-8-methyl-8-(1-methyl-1H-pyrazol-4-yl)-7,8-dihydro-6H-cyclopenta[e]pyrazolo[1,5-a]pyrimidine

To a stirred solution of 5-((dimethylamino)methylene)-2-methyl-2-(1-methyl-1H-pyrazol-4-yl)cyclopentan-1-one (A1-4, 2.2 g, 9.43 mmol) in Toluene (15 mL) were added 3-chloro-1H-pyrazol-5-amine (1.3 g, 11.3 mmol) and AcOH (1.5 mL). The resulting mixture was stirred at 110° C. for 24 h. The mixture was allowed to cool to 25° C. The reaction mixture was concentrated under reduced pressure, and the residue was applied onto a silica gel column and eluted with EtOAc/PE (10:1) to give 2-chloro-8-methyl-8-(1-methyl-1H-pyrazol-4-yl)-7,8-dihydro-6H-cyclopenta[e]pyrazolo[1,5-a]pyrimidine (1.5 g, 44% yield) as a light yellow solid. LC-MS: m/z 288 [M+H]⁺.

### Step 2: 2-chloro-8-methyl-8-(1-methyl-1H-pyrazol-4-yl)-7,8-dihydro-6H-cyclopenta[e]pyrazolo[1,5-a]pyrimidine-6-carbonitrile

To a stirred solution of 2-chloro-8-methyl-8-(1-methyl-1H-pyrazol-4-yl)-7,8-dihydro-6H-cyclopenta[e]pyrazolo[1,5-a]pyrimidine (1.1 g, 3.82 mmol) in toluene (40 mL) were added (4R)-4-benzyl-2-[1-[(4R)-4-benzyl-4,5-dihydrooxazol-2-yl]-1-methyl-ethyl]-4,5-dihydrooxazole (1.4 g, 3.8 mmol), acetoxycopper (94 mg, 764 μmol), N-Fluorobenzenesulfonimide (1.8 g, 5.7 mmol) and TMSCN (1.9 g, 19.1 mmol). The reaction was stirred at 25° C. for 24 h under nitrogen. The solvent was removed under reduced pressure. The residue was diluted with water (100 mL). The resulting mixture was extracted with ethyl acetate (3×100 mL). The combined organic layers were dried over anhydrous sodium sulfate and concentrated under reduced pressure. The residue was applied on a silica gel column and eluted with EtOAc/PE (1:1) to give crude product, which was further purified by HPLC to give the title compound (240 mg, 17% yield) as a yellow oil. LC-MS (ES, m/z): 313[M+H]⁺.

### Step 3: 2-chloro-8-methyl-8-(1-methyl-1H-pyrazol-4-yl)-7,8-dihydro-6H-cyclopenta[e]pyrazolo[1,5-a]pyrimidine-6-carboxylic acid (A3-3)

In analogy to Method A1 step 7 but using 2-chloro-8-methyl-8-(1-methyl-1H-pyrazol-4-yl)-7,8-dihydro-6H-cyclopenta[e]pyrazolo[1,5-a]pyrimidine-6-carbonitrile (240 mg, 0.77 mmol), the title compound was obtained as a yellow oil (A3-3, 235 mg, 94% yield). LC-MS: m/z 332 [M+H]⁺.

### Step 4: trans-2-chloro-N-(5-chloro-6-(2H-1,2,3-triazol-2-yl)pyridin-3-yl)-8-methyl-8-(1-methyl-1H-pyrazol-4-yl)-7,8-dihydro-6H-cyclopenta[e]pyrazolo[1,5-a]pyrimidine-6-carboxamide and cis-2-chloro-N-(5-chloro-6-(2H-1,2,3-triazol-2-yl)pyridin-3-yl)-8-methyl-8-(1-methyl-1H-pyrazol-4-yl)-7,8-dihydro-6H-cyclopenta[e]pyrazolo[1,5-a]pyrimidine-6-carboxamide

In analogy to Method A1 step 8 but using 2-chloro-8-methyl-8-(1-methyl-1H-pyrazol-4-yl)-7,8-dihydro-6H-cyclopenta[e]pyrazolo[1,5-a]pyrimidine-6-carboxylic acid (120 mg, 362 μmol) and 5-chloro-6-(triazol-2-yl)pyridin-3-amine (141 mg, 723 μmol) and stirring at 25° C. for 24 h, racemic mixtures of the title compounds were obtained: trans isomer (25 mg, 13% yield) as a white solid, LC-MS: m/z 509 [M+H]⁺; cis-isomer (15 mg, 8% yield) as a white solid, LC-MS: m/z 509 [M+H]⁺.

### Step 5: Separation of Enantiomers to Obtain Example 8 and Example 9

25 mg of trans-2-chloro-N-(5-chloro-6-(2H-1,2,3-triazol-2-yl)pyridin-3-yl)-8-methyl-8-(1-methyl-1H-pyrazol-4-yl)-7,8-dihydro-6H-cyclopenta[e]pyrazolo[1,5-a]pyrimidine-6-carboxamide were submitted to chiral HPLC purification (Column: CHIRALPAK IF, 2*25 cm, 5 μm; Mobile Phase A: Hex (0.1% FA)-HPLC, Mobile Phase B: EtOH-HPLC; Flow rate: 20 mL/min; Gradient: isocratic 50% Mobile Phase B; Wave Length: 220/254 nm; RT1 (min): 13.764; RT2 (min): 19.041; Sample Solvent: EtOH-HPLC; Injection Volume: 2 mL; Number Of Runs: 2). The second eluting isomer was concentrated and lyophilized to afford Example 8 (8.9 mg, 43% yield) as a white solid. The first eluting isomer was concentrated and lyophilized to afford Example 9 (8.4 mg, 40% yield) as a white solid.

Example 8: ¹H NMR (300 MHz, DMSO-d₆) δ 11.10 (s, 1H), 8.72 (d, J=2.1 Hz, 1H), 8.68 (s, 1H), 8.61 (d, J=2.1 Hz, 1H), 8.18 (s, 2H), 7.63 (s, 1H), 7.41 (s, 1H), 6.96 (s, 1H), 4.51-4.57 (m, 1H), 3.76 (s, 3H), 2.86-2.93 (m, 1H), 2.50-2.51 (m, 1H), 1.99 (s, 3H). LC-MS: m/z 509 [M+H]⁺.

Example 9: ¹H NMR (300 MHz, DMSO-d₆) δ 11.10 (s, 1H), 8.72 (d, J=2.1 Hz, 1H), 8.68 (s, 1H), 8.61 (d, J=2.1 Hz, 1H), 8.18 (s, 2H), 7.63 (s, 1H), 7.41 (s, 1H), 6.96 (s, 1H), 4.50-4.54 (m, 1H), 3.76 (s, 3H), 2.86-2.93 (m, 1H), 2.50-2.51 (m, 1H), 1.99 (s, 3H). LC-MS: m/z 509 [M+H]⁺.

### Step 6: Separation of Enantiomers to Obtain Example 10 and Example 11

15 mg of cis-2-chloro-N-(5-chloro-6-(2H-1,2,3-triazol-2-yl)pyridin-3-yl)-8-methyl-8-(1-methyl-1H-pyrazol-4-yl)-7,8-dihydro-6H-cyclopenta[e]pyrazolo[1,5-a]pyrimidine-6-carboxamide were submitted to chiral HPLC purification (Column: CHIRALPAK IF, 2*25 cm, 5 m; Mobile Phase A: Hex (0.1% FA)-HPLC, Mobile Phase B: EtOH-HPLC; Flow rate: 20 mL/min; Gradient: isocratic 50% Mobile Phase B; Wave Length: 220/254 nm; RT1 (min): 14.519; RT2 (min): 26.523; Sample Solvent: EtOH-HPLC; Injection Volume: 2 mL; Number Of Runs: 2). The first eluting isomer was concentrated and lyophilized to afford Example 10 (2.4 mg, 15% yield) as a white solid. The second eluting isomer was concentrated and lyophilized to afford Example 11(1.7 mg, 11% yield) as a white solid.

Example 10: ¹H NMR (300 MHz, DMSO-d₆) δ 11.09 (s, 1H), 8.70-8.72 (m, 2H), 8.57 (d, J=2.4 Hz, 1H), 8.17 (s, 2H), 7.61 (s, 1H), 7.34 (s, 1H), 6.94 (s, 1H), 4.51-4.56 (m, 1H), 3.75 (s, 3H), 2.66-2.88 (m, 2H), 1.89 (s, 3H). LC-MS: m/z 509 [M+H]⁺.

Example 11: ¹H NMR (300 MHz, DMSO-d₆) δ 11.09 (s, 1H), 8.70-8.72 (m, 2H), 8.57 (d, J=2.4 Hz, 1H), 8.17 (s, 2H), 7.61 (s, 1H), 7.34 (s, 1H), 6.94 (s, 1H), 4.51-4.56 (m, 1H), 3.75 (s, 3H), 2.67-2.88 (m, 2H), 1.89 (s, 3H). LC-MS: m/z 509 [M+H]⁺.

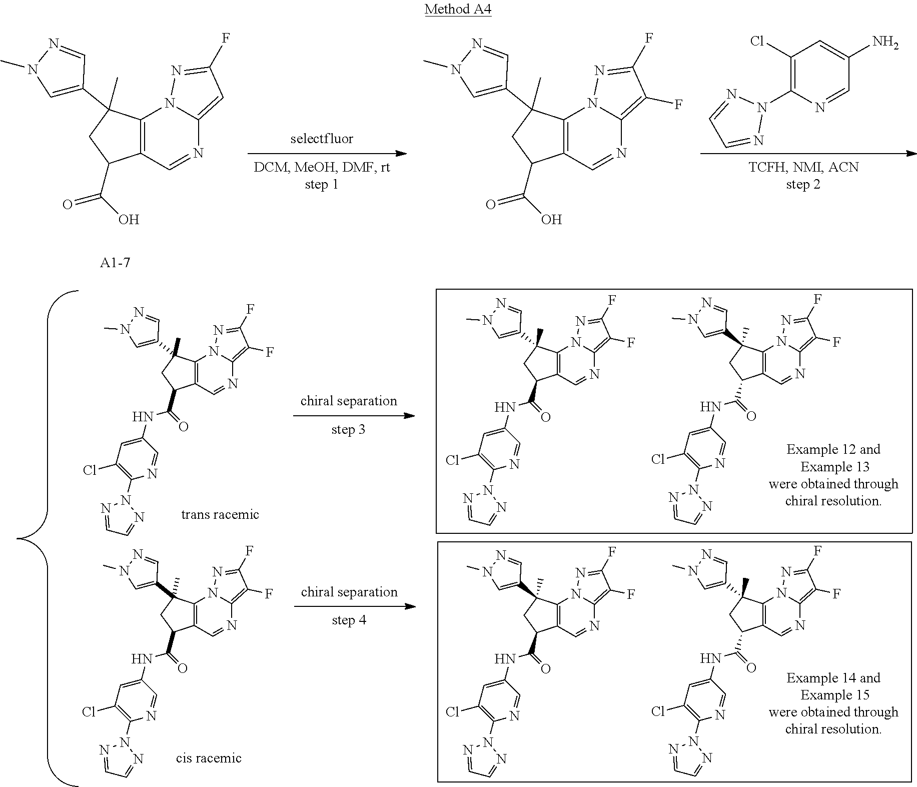

### Examples 12, 13: (6S,8R)—N-(5-chloro-6-(2H-1,2,3-triazol-2-yl)pyridin-3-yl)-2,3-difluoro-8-methyl-8-(1-methyl-1H-pyrazol-4-yl)-7,8-dihydro-6H-cyclopenta[e]pyrazolo[1,5-a]pyrimidine-6-carboxamide; (6R,8S)—N-(5-chloro-6-(2H-1,2,3-triazol-2-yl)pyridin-3-yl)-2,3-difluoro-8-methyl-8-(1-methyl-1H-pyrazol-4-yl)-7,8-dihydro-6H-cyclopenta[e]pyrazolo[1,5-a]pyrimidine-6-carboxamide

### Examples 14, 15: (6S,8S)—N-(5-chloro-6-(2H-1,2,3-triazol-2-yl)pyridin-3-yl)-2,3-difluoro-8-methyl-8-(1-methyl-1H-pyrazol-4-yl)-7,8-dihydro-6H-cyclopenta[e]pyrazolo[1,5-a]pyrimidine-6-carboxamide; (6R,8R)—N-(5-chloro-6-(2H-1,2,3-triazol-2-yl)pyridin-3-yl)-2,3-difluoro-8-methyl-8-(1-methyl-1H-pyrazol-4-yl)-7,8-dihydro-6H-cyclopenta[e]pyrazolo[1,5-a]pyrimidine-6-carboxamide

The relative and absolute stereochemistry of each example was not determined.

### Step 1: 2,3-difluoro-8-methyl-8-(1-methyl-1H-pyrazol-4-yl)-7,8-dihydro-6H-cyclopenta[e]pyrazolo[1,5-a]pyrimidine-6-carboxylic acid

To a solution of 2-fluoro-8-methyl-8-(1-methyl-1H-pyrazol-4-yl)-7,8-dihydro-6H-cyclopenta[e]pyrazolo[1,5-a]pyrimidine-6-carboxylic acid (A1-7, 592 mg, 1.88 mmol) in DCM (18 mL) and MeOH (18 mL) was added Selectfluor fluorinating reagent (2.0 g, 5.63 mmol) in DMF (6 mL). The reaction was stirred at 25° C. for 16 h under nitrogen. The solvent was concentrated under reduced pressure and the residue was diluted with water (50 ml). The resulting solution was extracted with ethyl acetate (3×50 mL). The combined organic layers were dried over anhydrous sodium sulfate and concentrated under reduced pressure. The crude product was purified by prep-HPLC to give the title compound (130 mg, 12% yield) as a yellow oil. LC-MS: m/z 334 [M+H]⁺.

### Step 2: trans-N-(5-chloro-6-(2H-1,2,3-triazol-2-yl)pyridin-3-yl)-2,3-difluoro-8-methyl-8-(1-methyl-1H-pyrazol-4-yl)-7,8-dihydro-6H-cyclopenta[e]pyrazolo[1,5-a]pyrimidine-6-carboxamide and cis-N-(5-chloro-6-(2H-1,2,3-triazol-2-yl)pyridin-3-yl)-2,3-difluoro-8-methyl-8-(1-methyl-1H-pyrazol-4-yl)-7,8-dihydro-6H-cyclopenta[e]pyrazolo[1,5-a]pyrimidine-6-carboxamide

In analogy to Method A1 step 8 but using 2,3-difluoro-8-methyl-8-(1-methyl-1H-pyrazol-4-yl)-7,8-dihydro-6H-cyclopenta[e]pyrazolo[1,5-a]pyrimidine-6-carboxylic acid (124 mg, 372 μmol) and 5-chloro-6-(2H-1,2,3-triazol-2-yl)pyridin-3-amine (109 mg, 557 μmol), racemic mixtures of the title compounds were obtained: trans isomer (50 mg, 25% yield) as a white solid, LC-MS: m/z 511 [M+H]⁺; cis-isomer (25 mg, 13% yield) as a white solid, LC-MS: m/z 511 [M+H]⁺.

### Step 3: Separation of Enantiomers to Obtain Example 12 and Example 13

50 mg of trans-N-(5-chloro-6-(2H-1,2,3-triazol-2-yl)pyridin-3-yl)-2,3-difluoro-8-methyl-8-(1-methyl-1H-pyrazol-4-yl)-7,8-dihydro-6H-cyclopenta[e]pyrazolo[1,5-a]pyrimidine-6-carboxamide were submitted to chiral HPLC purification (Column: CHIRAL ART Amylose-SA, 2*25 cm, 5 m; Mobile Phase A: Hex (0.1% FA)-HPLC, Mobile Phase B: EtOH-HPLC; Flow rate: 20 mL/min; Gradient: isocratic 30% Mobile Phase B; Wave Length: 220/254 nm; RT1 (min): 9.925; RT2 (min): 16.35; Sample Solvent: EtOH-HPLC; Injection Volume: 1 mL; Number Of Runs: 6). The second eluting isomer was concentrated and lyophilized to afford Example 12 (22.5 mg, 43% yield) as a white solid. The first eluting isomer was concentrated and lyophilized to afford Example 13 (11.6 mg, 23% yield) as a white solid.

Example 12: ¹H NMR (400 MHz, DMSO-d₆) δ 11.08 (s, 1H), 8.72 (d, J=2.4 Hz, 1H), 8.71 (s, 1H), 8.61 (d, J=2.4 Hz, 1H), 8.18 (s, 2H), 7.64 (s, 1H), 7.42 (s, 1H), 4.53 (t, J=8.4 Hz, 1H), 3.74 (s, 3H), 2.87-2.93 (m, 1H), 2.50-2.56 (m, 1H), 1.93 (s, 3H). LC-MS: m/z 511 [M+H]⁺.

Example 13: ¹H NMR (400 MHz, DMSO-d₆) δ 11.08 (s, 1H), 8.72 (d, J=2.4 Hz, 1H), 8.71 (s, 1H), 8.61 (d, J=2.4 Hz, 1H), 8.18 (s, 2H), 7.64 (s, 1H), 7.42 (s, 1H), 4.53 (t, J=8.4 Hz, 1H), 3.74 (s, 3H), 2.87-2.93 (m, 1H), 2.50-2.56 (m, 1H), 1.93 (s, 3H). LC-MS: m/z 511 [M+H]⁺.

### Step 4: Separation of Enantiomers to Obtain Example 14 and Example 15

25 mg of cis-N-(5-chloro-6-(2H-1,2,3-triazol-2-yl)pyridin-3-yl)-2,3-difluoro-8-methyl-8-(1-methyl-1H-pyrazol-4-yl)-7,8-dihydro-6H-cyclopenta[e]pyrazolo[1,5-a]pyrimidine-6-carboxamide were submitted to chiral HPLC purification (Column: CHIRAL ART Amylose-SA, 2*25 cm, 5 m; Mobile Phase A: Hex (0.1% FA)-HPLC, Mobile Phase B: EtOH-HPLC; Flow rate: 20 mL/min; Gradient: isocratic 30% Mobile Phase B; Wave Length: 220/254 nm; RT1 (min): 9.465; RT2 (min): 13.025; Sample Solvent: EtOH-HPLC; Injection Volume: 1 mL; Number Of Runs: 4). The second eluting isomer was concentrated and lyophilized to afford Example 14 (9.8 mg, 38% yield) as a white solid. The first eluting isomer was concentrated and lyophilized to afford Example 15 (6.4 mg, 25% yield) as a white solid.

Example 14: ¹H NMR (400 MHz, DMSO-d₆) δ 11.09 (s, 1H), 8.74 (s, 1H), 8.72 (d, J=2.4 Hz, 1H), 8.56 (d, J=2.4 Hz, 1H), 8.17 (s, 2H), 7.62 (s, 1H), 7.34 (s, 1H), 4.52-4.56 (m, 1H), 3.74 (s, 3H), 2.81-2.87 (m, 1H), 2.66-2.71 (m, 1H), 1.86 (s, 3H). LC-MS: m/z 511 [M+H]⁺.

Example 15: ¹H NMR (400 MHz, DMSO-d₆) δ 11.08 (s, 1H), 8.74 (s, 1H), 8.72 (d, J=2.4 Hz, 1H), 8.56 (d, J=2.4 Hz, 1H), 8.17 (s, 2H), 7.62 (s, 1H), 7.34 (s, 1H), 4.52-4.56 (m, 1H), 3.74 (s, 3H), 2.81-2.87 (m, 1H), 2.66-2.71 (m, 1H), 1.86 (s, 3H). LC-MS: m/z 511 [M+H]⁺.

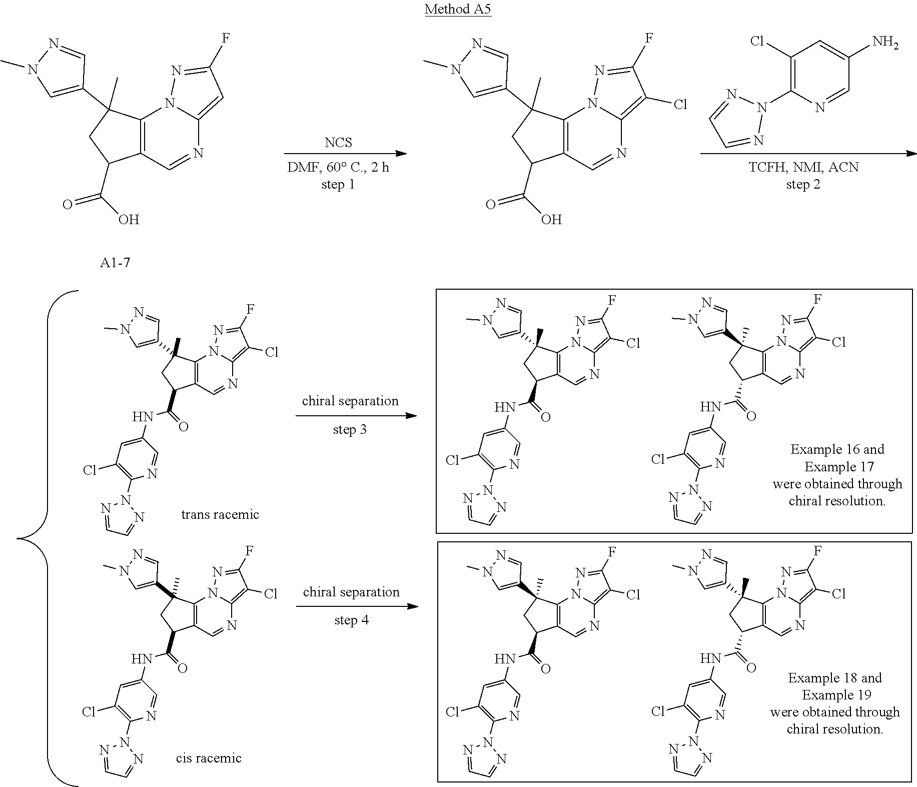

### Examples 16, 17: (6S,8R)-3-chloro-N-(5-chloro-6-(2H-1,2,3-triazol-2-yl) pyridin-3-yl)-2-fluoro-8-methyl-8-(1-methyl-1H-pyrazol-4-yl)-7,8-dihydro-6H-cyclopenta[e]pyrazolo[1,5-a]pyrimidine-6-carboxamide; (6R,8S)-3-chloro-N-(5-chloro-6-(2H-1,2,3-triazol-2-yl) pyridin-3-yl)-2-fluoro-8-methyl-8-(1-methyl-1H-pyrazol-4-yl)-7,8-dihydro-6H-cyclopenta[e]pyrazolo[1,5-a]pyrimidine-6-carboxamide

### Examples 18, 19: (65,8S)-3-chloro-N-(5-chloro-6-(2H-1,2,3-triazol-2-yl) pyridin-3-yl)-2-fluoro-8-methyl-8-(1-methyl-1H-pyrazol-4-yl)-7,8-dihydro-6H-cyclopenta[e]pyrazolo[1,5-(6R,8R)-3-chloro-N-(5-chloro-6-(2H-1,2,3-triazol-2-a]pyrimidine-6-carboxamide; yl) pyridin-3-yl)-2-fluoro-8-methyl-8-(1-methyl-1H-pyrazol-4-yl)-7,8-dihydro-6H-cyclopenta[e]pyrazolo[1,5-a]pyrimidine-6-carboxamide

The relative and absolute stereochemistry of each example was not determined.

### Step 1:3-chloro-2-fluoro-8-methyl-8-(1-methyl-1H-pyrazol-4-yl)-7,8-dihydro-6H-cyclopenta[e]pyrazolo[1,5-a]pyrimidine-6-carboxylic acid

To a solution of 2-fluoro-8-methyl-8-(1-methyl-1H-pyrazol-4-yl)-7,8-dihydro-6H-cyclopenta[e]pyrazolo[1,5-a]pyrimidine-6-carboxylic acid (A1-7, 100 mg, 317 mol) in DMF (10 mL) was added NCS (85 mg, 634 mol). The reaction was stirred at 60° C. for 2 h under nitrogen. The reaction was allowed to cool to 25° C. The reaction was diluted with water (50 ml), and the resulting mixture was extracted with ethyl acetate (3×50 mL). The combined organic layers were washed with brine (3×50 mL), dried over anhydrous sodium sulfate and concentrated under reduced pressure. The residue was purified by HPLC to give the title compound (100 mg, 81% yield) as a yellow oil. LC-MS: m/z 350 [M+H]⁺.

### Step 2: trans-3-chloro-N-(5-chloro-6-(2H-1,2,3-triazol-2-yl)pyridin-3-yl)-2-fluoro-8-methyl-8-(1-methyl-1H-pyrazol-4-yl)-7,8-dihydro-6H-cyclopenta[e]pyrazolo[1,5-a]pyrimidine-6-carboxamide and cis-3-chloro-N-(5-chloro-6-(2H-1,2,3-triazol-2-yl)pyridin-3-yl)-2-fluoro-8-methyl-8-(1-methyl-1H-pyrazol-4-yl)-7,8-dihydro-6H-cyclopenta[e]pyrazolo[1,5-a]pyrimidine-6-carboxamide

In analogy to Method A1 step 8 but using 3-chloro-2-fluoro-8-methyl-8-(1-methyl-1H-pyrazol-4-yl)-7,8-dihydro-6H-cyclopenta[e]pyrazolo[1,5-a]pyrimidine-6-carboxylic acid (130 mg, 372 μmol) and 5-chloro-6-(2H-1,2,3-triazol-2-yl)pyridin-3-amine (110 mg, 556 μmol), racemic mixtures of the title compounds were obtained: trans isomer (35 mg, 17% yield) as a white solid, LC-MS: m/z 527 [M+H]⁺; cis-isomer (25 mg, 12% yield) as a white solid, LC-MS: m/z 527 [M+H]⁺.

### Step 3: Separation of Enantiomers to Obtain Example 16 and Example 17

35 mg of trans-3-chloro-N-(5-chloro-6-(2H-1,2,3-triazol-2-yl)pyridin-3-yl)-2-fluoro-8-methyl-8-(1-methyl-1H-pyrazol-4-yl)-7,8-dihydro-6H-cyclopenta[e]pyrazolo[1,5-a]pyrimidine-6-carboxamide were submitted to chiral HPLC purification (Column: CHIRALPAK IF, 2*25 cm, 5 μm; Mobile Phase A: Hex (0.1% FA)-HPLC, Mobile Phase B: IPA-HPLC; Flow rate: 20 mL/min; Gradient: isocratic 40% Mobile Phase B; Wave Length: 220/254 nm; RT1 (min): 12.633; RT2 (min): 17.731; Sample Solvent: EtOH-HPLC; Injection Volume: 0.5 mL; Number Of Runs: 6). The second eluting isomer was concentrated and lyophilized to afford Example 16 (7.1 mg, 19% yield) as a white solid. The first eluting isomer was concentrated and lyophilized to afford Example 17 (11.5 mg, 31% yield) as a white solid.

Example 16: ¹H NMR (400 MHz, DMSO-d₆) δ 11.17 (s, 1H), 8.77 (s, 1H), 8.73 (d, J=2.0 Hz, 1H), 8.62 (d, J=2.0 Hz, 1H), 8.18 (s, 2H), 7.64 (s, 1H), 7.44 (s, 1H), 4.56 (t, J=8.4 Hz, 1H), 3.75 (s, 3H), 2.90-2.95 (m, 1H), 2.50-2.56 (m, 1H), 1.95 (s, 3H). LC-MS: m/z 527 [M+H]⁺.

Example 17: ¹H NMR (400 MHz, DMSO-d₆) δ 11.22 (s, 1H), 8.77 (s, 1H), 8.73 (d, J=2.0 Hz, 1H), 8.62 (d, J=2.0 Hz, 1H), 8.18 (s, 2H), 7.64 (s, 1H), 7.44 (s, 1H), 4.55 (t, J=8.4 Hz, 1H), 3.75 (s, 3H), 2.90-2.95 (m, 1H), 2.50-2.56 (m, 1H), 1.95 (s, 3H). LC-MS: m/z 527 [M+H]⁺.

### Step 4: Separation of Enantiomers to Obtain Example 18 and Example 19

25 mg of cis-3-chloro-N-(5-chloro-6-(2H-1,2,3-triazol-2-yl)pyridin-3-yl)-2-fluoro-8-methyl-8-(1-methyl-1H-pyrazol-4-yl)-7,8-dihydro-6H-cyclopenta[e]pyrazolo[1,5-a]pyrimidine-6-carboxamide were submitted to chiral HPLC purification (Column: CHIRAL ART Amylose-SA, 2*25 cm, 5 m; Mobile Phase A: Hex (0.1% FA)-HPLC, Mobile Phase B: IPA-HPLC; Flow rate: 20 mL/min; Gradient: isocratic 30% Mobile Phase B; Wave Length: 220/254 nm; RT1 (min): 8.045; RT2 (min): 9.61; Sample Solvent: EtOH-HPLC; Injection Volume: 0.5 mL; Number Of Runs: 6). The first eluting isomer was concentrated and lyophilized to afford Example 18 (4.1 mg, 15% yield) as a white solid. The second eluting isomer was concentrated and lyophilized to afford Example 19 (5.5 mg, 21% yield) as a white solid.

Example 18: ¹H NMR (400 MHz, DMSO-d₆) δ 11.11 (s, 1H), 8.81 (s, 1H), 8.73 (d, J=2.0 Hz, 1H), 8.56 (d, J=2.0 Hz, 1H), 8.18 (s, 2H), 7.62 (s, 1H), 7.35 (s, 1H), 4.52-4.58 (m, 1H), 3.74 (s, 3H), 2.82-2.94 (m, 1H), 2.67-2.72 (m, 1H), 1.86 (s, 3H). LC-MS: m/z 527 [M+H]⁺.

Example 19: ¹H NMR (400 MHz, DMSO-d₆) δ 11.11 (s, 1H), 8.81 (s, 1H), 8.73 (d, J=2.0 Hz, 1H), 8.56 (d, J=2.0 Hz, 1H), 8.18 (s, 2H), 7.62 (s, 1H), 7.35 (s, 1H), 4.53-4.58 (m, 1H), 3.74 (s, 3H), 2.82-2.94 (m, 111), 2.67-2.72 (m, 1H), 1.86 (s, 311). LC-MS: m/z 527 [M+H]⁺.

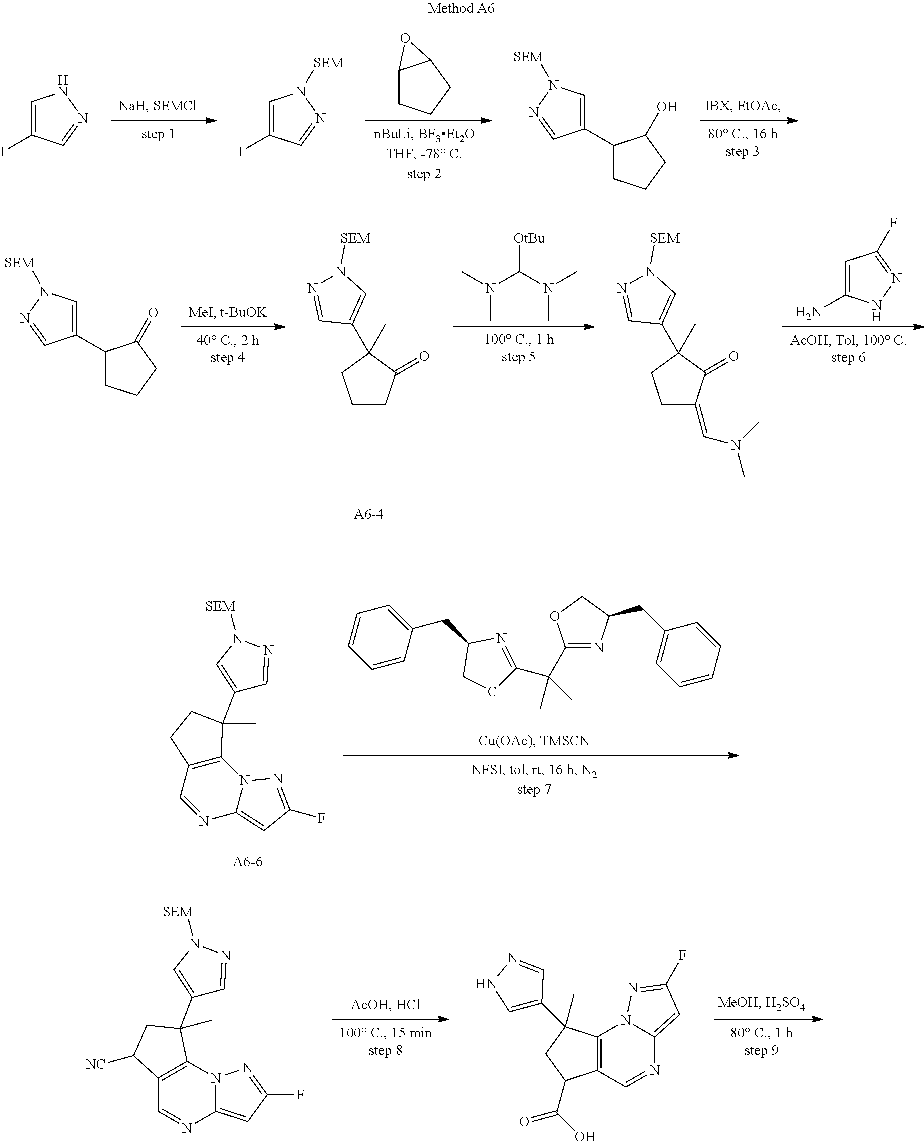

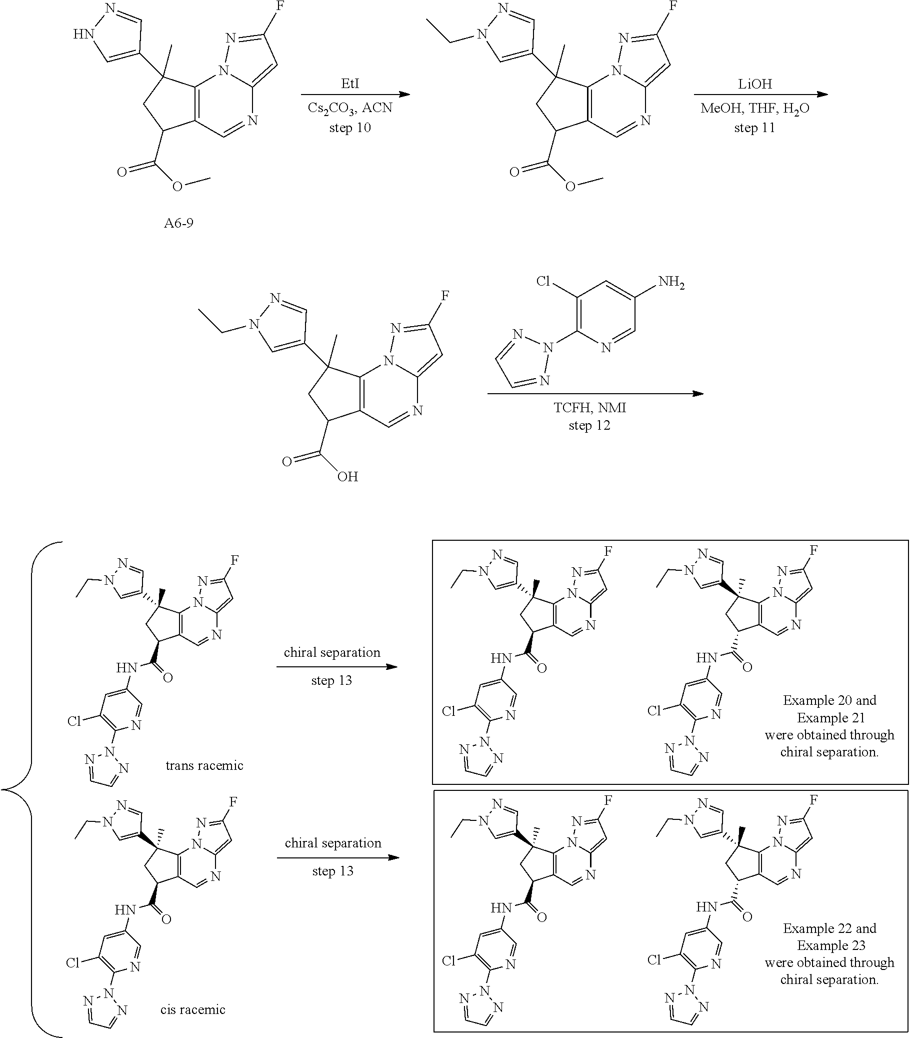

### Examples 20, 21: (6S,8R)—N-(5-chloro-6-(2H-1,2,3-triazol-2-yl)pyridin-3-yl)-8-(1-ethyl-1H-pyrazol-4-y)-2-fluoro-8-methyl-7,8-dihydro-6H-cyclopenta[e]pyrazolo[1,5-a]pyrimidine-6-carboxamide; (6R,8S)—N-(5-chloro-6-(2H-1,2,3-triazol-2-yl)pyridin-3-yl)-8-(1-ethyl-1H-pyrazol-4-yl)-2-fluoro-8-methyl-7,8-dihydro-6H-cyclopenta[e]pyrazolo[1,5-a]pyrimidine-6-carboxamide

### Examples 22, 23: (6S,8S)—N-(5-chloro-6-(2H-1,2,3-triazol-2-yl)pyridin-3-yl)-8-(1-ethyl-1H-pyrazol-4-yl)-2-fluoro-8-methyl-7,8-dihydro-6H-cyclopenta[e]pyrazolo[1,5-a]pyrimidine-6-carboxamide, (6R,8R)—N-(5-chloro-6-(2H-1,2,3-triazol-2-yl)pyridin-3-yl)-8-(1-ethyl-1H-pyrazol-4-yl)-2-fluoro-8-methyl-7,8-dihydro-6H-cyclopenta[e]pyrazolo[1,5-a]pyrimidine-6-carboxamide

The relative and absolute stereochemistry of each example was not determined.

### Step 1: 4-iodo-1-((2-(trimethylsilyl)ethoxy)methyl)-1H-pyrazole

To a stirred solution of 4-iodo-1H-pyrazole (100 g, 515.5 mmol) in THF (1.2 L) was added NaH (30.9 g, 773.3 mmol, 60% in mineral oil) at 0° C. The mixture was stirred at 0° C. for 30 min. 2-(chloromethoxy)ethyl-trimethyl-silane (128.9 g, 773.3 mmol) was added dropwise at 0° C., and the mixture was stirred at 25° C. for 2 h. The reaction mixture was poured into ice-water (2500 mL) and extracted with ethyl acetate (3×2500 mL). The combined organic layers were dried over anhydrous sodium sulfate and concentrated under reduced pressure. The residue was applied onto a silica gel column and eluted with EtOAc/PE (1:3) to give the title compound (130 g, 73% yield) as a yellow oil. LC-MS: m/z 325 [M+H]⁺.

### Step 2: 2-(1-((2-(trimethylsilyl)ethoxy)methyl)-1H-pyrazol-4-yl)cyclopentan-1-ol

To a stirred mixture of 4-iodo-1-((2-(trimethylsilyl)ethoxy)methyl)-1H-pyrazole (50.1 g, 154.6 mmol) in Et₂O (800 mL) was added n-Butyllithium (61.8 mL, 2.5M in hexane) at −90° C. under nitrogen. The reaction mixture was stirred at −90° C. for 1 h under nitrogen. 6-oxabicyclo[3.1.0]hexane (10 g, 118.9 mmol) and boron trifluoride diethyl etherate (21.9 g, 154.6 mmol) were added at −90° C., and the mixture was stirred at −78° C. for 1 h under nitrogen. The reaction was quenched with saturated aqueous ammonium chloride (100 mL), and the resulting mixture was extracted with ethyl acetate (3×200 mL). The combined organic layers were dried over anhydrous sodium sulfate and concentrated under reduced pressure. The residue was applied onto a silica gel column and eluted with EtOAc/PE (2:1) to afford the title compound (9.6 g, 25% yield) as a yellow oil. LC-MS: m/z 283 [M+H]⁺.

### Step 3: 2-(1-((2-(trimethylsilyl)ethoxy)methyl)-1H-pyrazol-4-yl)cyclopentan-1-one

In analogy to Method A1 step 2 but using 2-(1-((2-(trimethylsilyl)ethoxy)methyl)-1H-pyrazol-4-yl)cyclopentan-1-ol (9.6 g, 34.0 mmol), the title compound was obtained as a yellow oil (6.5 g, 61% yield). LC-MS: m/z 281 [M+H]⁺.

### Step 4: 2-methyl-2-(1-((2-(trimethylsilyl)ethoxy)methyl)-1H-pyrazol-4-yl)cyclopentan-1-one (A6-4)

In analogy to Method A1 step 3 but using 2-(1-((2-(trimethylsilyl)ethoxy)methyl)-1H-pyrazol-4-yl)cyclopentan-1-one (6.5 g, 23.2 mmol), the title compound was obtained as a yellow oil (A6-4, 3.4 g, 44% yield). ¹H NMR (400 MHz, DMSO-d₆) δ 7.73 (s, 1H), 7.43 (s, 1H), 5.33 (s, 2H), 3.43-3.55 (m, 2H), 2.24-2.34 (m, 3H), 1.76-2.01 (m, 3H), 1.25 (s, 3H), 0.78-0.83 (m, 2H), −0.06 (s, 9H). LC-MS: m/z 295 [M+H]⁺.

### Step 5: 5-((dimethylamino)methylene)-2-methyl-2-(1-((2-(trimethylsilyl)ethoxy)methyl)-1H-pyrazol-4-yl)cyclopentan-1-one

In analogy to Method A1 step 4 but using 2-methyl-2-(1-((2-(trimethylsilyl)ethoxy)methyl)-1H-pyrazol-4-yl)cyclopentan-1-one (3.3 g, 11.2 mmol) in 1-tert-butoxy-N,N,N′,N′-tetramethyl-methanediamine (4.63 mL, 22.4 mmol) and stirring at 100° C. for 1 h, the crude title compound was obtained as a brown oil (4.5 g) and was used in the next step without further purification. LC-MS: m/z 350 [M+H]⁺.

### Step 6: 2-fluoro-8-methyl-8-(1-((2-(trimethylsilyl)ethoxy)methyl)-1H-pyrazol-4-yl)-7,8-dihydro-6H-cyclopenta[e]pyrazolo[1,5-a]pyrimidine (A6-6)

To a stirred solution of 5-((dimethylamino)methylene)-2-methyl-2-(1-((2-(trimethylsilyl)ethoxy)methyl)-1H-pyrazol-4-yl)cyclopentan-1-one (3.9 g, 8.9 mmol) in toluene (60 mL) were added 3-fluoro-1H-pyrazol-5-amine (1.8 g, 17.9 mmol) and AcOH (6 mL). The resulting mixture was stirred at 90° C. for 16 h. The mixture was allowed to cool to 25° C. and concentrated under reduced pressure. The residue was diluted with water (200 mL). The resulting mixture was extracted with ethyl acetate (2×200 mL). The combined organic layers were dried over anhydrous sodium sulfate and concentrated under reduced pressure. The residue was applied onto a silica gel column and eluted with EtOAc/PE (1:1). The obtained product was further purified by prep-HPLC to give the title compound (A6-6), 3 g, 79% yield) as a yellow oil. ¹jH NMR (300 MHz, Chloroform-d) δ 8.44 (s, 1H), 7.60 (s, 1H), 7.50 (s, 1H), 6.20 (d, J=6.0 Hz, 1H), 5.31 (s, 2H), 3.50-3.55 (m, 2H), 3.04-3.21 (m, 2H), 2.61-2.69 (m, 1H), 2.34-2.44 (m, 1H), 1.99 (s, 3H), 0.84-0.90 (m, 2H), 0.05 (s, 9H). LC-MS: m/z 388 [M+H]⁺.

### Step 7: 2-fluoro-8-methyl-8-(1-((2-(trimethylsilyl)ethoxy)methyl)-1H-pyrazol-4-yl)-7,8-dihydro-6H-cyclopenta[e]pyrazolo[1,5-a]pyrimidine-6-carbonitrile

To a stirred solution of 2-fluoro-8-methyl-8-(1-((2-(trimethylsilyl)ethoxy)methyl)-1H-pyrazol-4-yl)-7,8-dihydro-6H-cyclopenta[e]pyrazolo[1,5-a]pyrimidine (4 g, 10.3 mmol) in toluene (20 mL) were added (4R)-4-benzyl-2-[1-[(4R)-4-benzyl-4,5-dihydrooxazol-2-yl]-1-methyl-ethyl]-4,5-dihydrooxazole (449 mg, 1.2 mmol), acetoxycopper (255 mg, 2.1 mmol), N-fluorobenzenesulfonimide (4.9 g, 15.5 mmol) and TMSCN (5.1 g, 51.6 mmol). The mixture was stirred at 25° C. for 16 h under nitrogen. The reaction was quenched by the addition of water (100 mL), and the resulting mixture was extracted with ethyl acetate (2×100 mL). The combined organic layers were dried over anhydrous sodium sulfate and concentrated under reduced pressure. The residue was purified by prep-HPLC to give the title compound (1.5 g, 26% yield) as a yellow oil. LC-MS: m/z 413 [M+H]⁺.

### Step 8: 2-fluoro-8-methyl-8-(1H-pyrazol-4-yl)-7,8-dihydro-6H-cyclopenta[e]pyrazolo[1,5-a]pyrimidine-6-carboxylic acid

A solution of 2-fluoro-8-methyl-8-(1-((2-(trimethylsilyl)ethoxy)methyl)-1H-pyrazol-4-yl)-7,8-dihydro-6H-cyclopenta[e]pyrazolo[1,5-a]pyrimidine-6-carbonitrile (400 mg, 969 μmol) in AcOH (3 mL) and HCl (12M, 3 mL) was stirred at 90° C. for 15 min. The mixture was allowed to cool to 25° C. The pH was adjusted to ˜6 by careful addition of 2N sodium hydroxide. The mixture was then directly purified by reverse phase flash chromatography to give the title compound (300 mg, 77% yield) as a yellow oil. LC-MS: m/z 302 [M+H]⁺.

### Step 9: Methyl 2-fluoro-8-methyl-8-(1H-pyrazol-4-yl)-7,8-dihydro-6H-cyclopenta[e]pyrazolo[1,5-a]pyrimidine-6-carboxylate (A6-9)

To a stirred solution of 2-fluoro-8-methyl-8-(1H-pyrazol-4-yl)-7,8-dihydro-6H-cyclopenta[e]pyrazolo[1,5-a]pyrimidine-6-carboxylic acid (600 mg, 1.9 mmol) in MeOH (10 mL) were added two drops of conc. H₂SO₄. The mixture was stirred at 80° C. for 1 h. The reaction was quenched with water (100 mL), and the resulting mixture was extracted with ethyl acetate (3×100 mL). The combined organic layers were dried over anhydrous sodium sulfate and concentrated under reduced pressure to give the crude title compound (A6-9, 400 mg, 51% yield) as a yellow oil. LC-MS: m/z 316 [M+H]⁺.

### Step 10: Methyl 8-(1-ethyl-1H-pyrazol-4-yl)-2-fluoro-8-methyl-7,8-dihydro-6H-cyclopenta[e]pyrazolo[1,5-a]pyrimidine-6-carboxylate

To a stirred solution of methyl 2-fluoro-8-methyl-8-(1H-pyrazol-4-yl)-7,8-dihydro-6H-cyclopenta[e]pyrazolo[1,5-a]pyrimidine-6-carboxylate (200 mg, 634 mol) in ACN (5 mL) were added cesium carbonate (1.0 g, 3.2 mmol) and iodoethane (495 mg, 3.2 mmol). The mixture was stirred at 25° C. for 1 h. The reaction was quenched with water (20 mL), and the resulting mixture was extracted with ethyl acetate (3×20 mL). The combined organic layers were dried over anhydrous sodium sulfate and concentrated under reduced pressure. The residue was applied on a silica gel column and eluted with EtOAc/PE (1:1) to give the title compound (120 mg, 49% yield) as a yellow oil. LC-MS: m/z 344 [M+H]⁺.

### Step 11: 8-(1-ethyl-1H-pyrazol-4-yl)-2-fluoro-8-methyl-7,8-dihydro-6H-cyclopenta[e]pyrazolo[1,5-a]pyrimidine-6-carboxylic acid

To a stirred solution of methyl 8-(1-ethyl-1H-pyrazol-4-yl)-2-fluoro-8-methyl-7,8-dihydro-6H-cyclopenta[e]pyrazolo[1,5-a]pyrimidine-6-carboxylate (120 mg, 349 μmol) in MeOH (2 mL) and THF (2 mL) was added LiOH (42 mg, 1.8 mmol) in water (2 mL). The mixture was stirred at 25° C. for 1 h. The pH was adjusted to ˜5 with 2M HCl. The resulting mixture was extracted with ethyl acetate (3×20 mL). The combined organic layers were dried over anhydrous sodium sulfate and concentrated under reduced pressure to afford the crude title compound (100 mg, 78% yield) as a yellow oil. LC-MS: m/z 330 [M+H]⁺.

### Step 12: (trans)-N-(5-chloro-6-(2H-1,2,3-triazol-2-yl)pyridin-3-yl)-8-(1-ethyl-1H-pyrazol-4-yl)-2-fluoro-8-methyl-7,8-dihydro-6H-cyclopenta[e]pyrazolo[1,5-a]pyrimidine-6-carboxamide and (cis)-N-(5-chloro-6-(2H-1,2,3-triazol-2-yl)pyridin-3-yl)-8-(1-ethyl-1H-pyrazol-4-yl)-2-fluoro-8-methyl-7,8-dihydro-6H-cyclopenta[c]pyrazolo[1,5-a]pyrimidine-6-carboxamide

In analogy to Method A1 step 8 but using 8-(1-ethyl-1H-pyrazol-4-yl)-2-fluoro-8-methyl-7,8-dihydro-6H-cyclopenta[e]pyrazolo[1,5-a]pyrimidine-6-carboxylic acid (100 mg, 304 μmol) and 5-chloro-6-(triazol-2-yl)pyridin-3-amine (57 mg, 293 mol), racemic mixtures of the title compounds were obtained: trans isomer (60 mg, 37% yield) as a white solid, LC-MS: m/z 507 [M+H]⁺; cis-isomer (40 mg, 24% yield) as a white solid, LC-MS: m/z 507 [M+H]⁺.

### Step 13: Separation of Enantiomers to Obtain Example 20 and Example 21

60 mg of (trans)-N-(5-chloro-6-(2H-1,2,3-triazol-2-yl)pyridin-3-yl)-8-(1-ethyl-11H-pyrazol-4-yl)-2-fluoro-8-methyl-7,8-dihydro-6H-cyclopenta[e]pyrazolo[1,5-a]pyrimidine-6-carboxamide were submitted to chiral HPLC purification. (Column: CHIRALPAK IA, 2*25 cm, 5 μm; Mobile Phase A: Hex (0.1% FA)-HPLC, Mobile Phase B: IPA-HPLC; Flow rate: 20 mL/min; Gradient: isocratic 40% Mobile Phase B; Wave Length: 220/254 nm; RT1 (min): 10.081; RT2 (min): 18.654; Sample Solvent: EtOH-HPLC; Injection Volume: 1.5 mL; Number Of Runs: 2). The second eluting isomer was concentrated and lyophilized to afford Example 20 (25.7 mg, 42% yield) as a white solid. The first eluting isomer was concentrated and lyophilized to afford Example 21 (22.6 mg, 37% yield) as a white solid.

Example 20: ¹H NMR (300 MHz, Chloroform-d) δ 9.07 (br, 1H), 9.73 (s, 1H), 8.61 (s, 1H), 8.53 (s, 1H), 7.89-7.94 (m, 3H), 7.76 (s, 1H), 6.29 (d, J=5.1 Hz, 1H), 4.42-4.58 (m, 1H), 4.20-4.28 (m, 2H), 3.06-3.22 (m, 1H), 2.58-2.69 (m, 1H), 2.09 (s, 3H), 1.52 (t, J=6.9 Hz, 3H). LC-MS: m/z 507 [M+H]⁺.

Example 21: ¹H NMR (300 MHz, Chloroform-d) δ 9.08 (br, 1H), 8.73 (s, 1H), 8.61 (s, 1H), 8.54 (s, 1H), 7.90-7.94 (m, 3H), 7.77 (s, 1H), 6.29 (d, J=4.8 Hz, 1H), 4.43-4.58 (m, 1H), 4.20-4.28 (m, 2H), 3.05-3.22 (m, 1H), 2.58-2.69 (m, 1H), 2.09 (s, 311), 1.52 (t, J=6.9 Hz, 311). LC-MS: m/z 507 [M+H]⁺.

### Step 14: Separation of Enantiomers to Obtain Example 22 and Example 23

40 mg of (cis)-N-(5-chloro-6-(2H-1,2,3-triazol-2-yl)pyridin-3-yl)-8-(1-ethyl-1H-pyrazol-4-yl)-2-fluoro-8-methyl-7,8-dihydro-6H-cyclopenta[e]pyrazolo[1,5-a]pyrimidine-6-carboxamide was submitted to chiral HPLC purification (Column: CHIRALPAK ID, 2*25 cm, 5 m; Mobile Phase A: Hex (0.1% FA)-HPLC, Mobile Phase B: EtOH-HPLC; Flow rate: 20 mL/min; Gradient: isocratic 40% Mobile Phase B; Wave Length: 220/254 nm; RT1 (min): 13.051; RT2 (min): 20.088; Sample Solvent: EtOH-HPLC; Injection Volume: 2 mL; Number Of Runs: 2). The second eluting isomer was concentrated and lyophilized to afford Example 22 (14.1 mg, 35% yield) as a white solid. The first eluting isomer was concentrated and lyophilized to afford Example 23 (15.7 mg, 39% yield) as a white solid.

Example 22: ¹H NMR (300 MHz, Chloroform-d) δ 8.58 (s, 1H), 8.47 (s, 111). 8.30 (s, 1H), 7.93 (s, 2H), 7.82 (s, 1H), 7.60 (s, 1H), 7.48 (s, 1H), 6.30 (d, J=5.1 Hz, 1H), 4.34-4.42 (m, 1H), 4.08-4.17 (m, 2H), 2.83-3.03 (m, 2H), 2.00 (s, 3H), 1.43 (t, J=6.9 Hz, 3H). LC-MS: m/z 507 [M+H]⁺.

Example 23: ¹H NMR (300 MHz, Chloroform-d) δ 8.58 (s, 1H), 8.48 (s, 1H), 8.34 (s, 1H), 8.05 (s, 1H), 7.92 (s, 2H), 7.64 (s, 1H), 7.52 (s, 1H), 6.29 (d, J=5.1 Hz, 1H), 4.37-4.43 (m, 1H), 4.08-4.18 (m, 2H), 2.83-3.03 (m, 2H), 1.99 (s, 3H), 1.44 (t, J=6.9 Hz, 3H). LC-MS: m/z 507 [M+H]⁺.

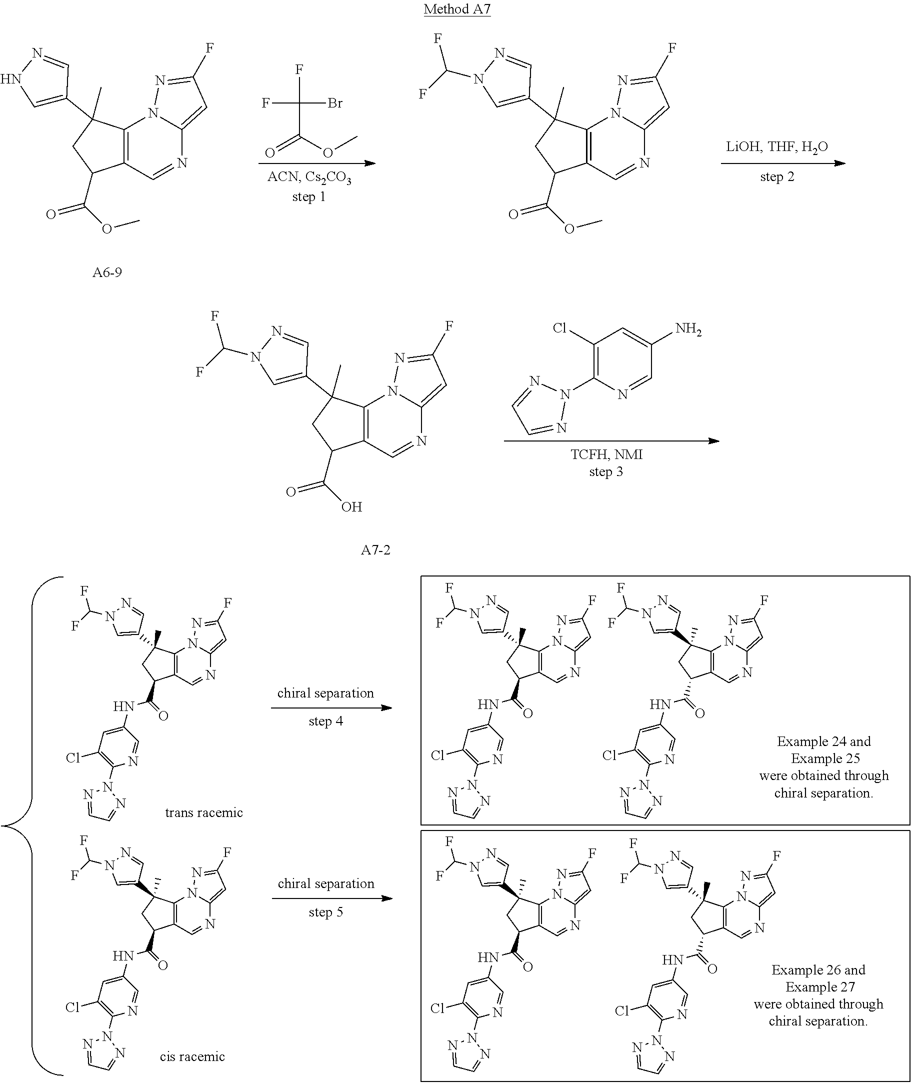

### Examples 24, 25: (6S,8R)—N-(5-chloro-6-(2H-1,2,3-triazol-2-yl)pyridin-3-yl)-8-(1-(difluoromethyl)-1H-pyrazol-4-yl)-2-fluoro-8-methyl-7,8-dihydro-6H-cyclopenta[e]pyrazolo[1,5-a]pyrimidine-6-carboxamide; (6R,8S)—N-(5-chloro-6-(2H-1,2,3-triazol-2-yl)pyridin-3-yl)-8-(1-(difluoromethyl)-1H-pyrazol-4-yl)-2-fluoro-8-methyl-7,8-dihydro-6H-cyclopenta[e]pyrazolo[1,5-a]pyrimidine-6-carboxamide

### Examples 26, 27: (6S,8S)—N-(5-chloro-6-(2H-1,2,3-triazol-2-yl)pyridin-3-yl)-8-(1-(difluoromethyl)-1H-pyrazol-4-yl)-2-fluoro-8-methyl-7,8-dihydro-6H-cyclopenta[e]pyrazolo[1,5-a]pyrimidine-6-carboxamide and (6R,8R)—N-(5-chloro-6-(2H-1,2,3-triazol-2-yl)pyridin-3-yl)-8-(1-(difluoromethyl)-1H-pyrazol-4-yl)-2-fluoro-8-methyl-7,8-dihydro-6H-cyclopenta[e]pyrazolo[1,5-a]pyrimidine-6-carboxamide

The relative and absolute stereochemistry of each example was not determined.

### Step 1: Methyl 8-(1-(difluoromethyl)-1H-pyrazol-4-yl)-2-fluoro-8-methyl-7,8-dihydro-6H-cyclopenta[e]pyrazolo[1,5-a]pyrimidine-6-carboxylate

To a stirred solution of methyl 2-fluoro-8-methyl-8-(1H-pyrazol-4-yl)-7,8-dihydro-6H-cyclopenta[e]pyrazolo[1,5-a]pyrimidine-6-carboxylate (A6-9, 200 mg, 634 mol) in ACN (6 mL) was added cesium carbonate (620 mg, 1.9 mmol) at 0° C. The mixture was stirred at 0° C. for 20 min. and a solution of methyl 2-bromo-2,2-difluoro-acetate (599.3 mg, 3.2 mmol) in ACN (0.5 mL) was added. The mixture was stirred at 25° C. for 16 h. The reaction was quenched with water (20 mL). The resulting mixture was extracted with ethyl acetate (3×20 mL). The combined organic layers were dried over anhydrous sodium sulfate and concentrated under reduced pressure. The residue was applied on a silica gel column and eluted with EtOAc/PE (1:1) to give the title compound (120 mg, 46% yield) as a yellow oil. LC-MS (ES, m/z): 366[M+H]⁺.

### Step 2: 8-(1-(difluoromethyl)-1H-pyrazol-4-yl)-2-fluoro-8-methyl-7,8-dihydro-6H-cyclopenta[e]pyrazolo[1,5-a]pyrimidine-6-carboxylic acid (A7-2)

To a stirred solution of methyl 8-(1-(difluoromethyl)-1H-pyrazol-4-yl)-2-fluoro-8-methyl-7,8-dihydro-6H-cyclopenta[e]pyrazolo[1,5-a]pyrimidine-6-carboxylate (120 mg, 328 mol) in MeOH (2 mL) and THF (2 mL) was added LiOH (39 mg, 1.6 mmol) in water (2 mL). The mixture was stirred at 25° C. for 1 h. The pH was adjusted to ˜4 with 1M HCl. The resulting mixture was extracted with ethyl acetate (3×20 mL). The combined organic layers were dried over anhydrous sodium sulfate and concentrated under reduced pressure to afford the crude title compound (A7-2, 100 mg, 78% yield) as a yellow oil. LC-MS (ES, m/z): 352[M+H]⁺.

### Step 3: (trans)-N-(5-chloro-6-(2H-1,2,3-triazol-2-yl)pyridin-3-yl)-8-(1-(difluoromethyl)-1H-pyrazol-4-yl)-2-fluoro-8-methyl-7,8-dihydro-6H-cyclopenta[e]pyrazolo[1,5-a]pyrimidine-6-carboxamide and (cis)-N-(5-chloro-6-(2H-1,2,3-triazol-2-yl)pyridin-3-yl)-8-(1-(difluoromethyl)-1H-pyrazol-4-yl)-2-fluoro-8-methyl-7,8-dihydro-6H-cyclopenta[e]pyrazolo[1,5-a]pyrimidine-6-carboxamide

In analogy to Method A1 step 8 but using 8-(1-(difluoromethyl)-1H-pyrazol-4-yl)-2-fluoro-8-methyl-7,8-dihydro-6H-cyclopenta[e]pyrazolo[1,5-a]pyrimidine-6-carboxylic acid (100 mg, 285 mol) and 5-chloro-6-(triazol-2-yl)pyridin-3-amine (210 mg, 1.1 mmol), racemic mixtures of the title compounds were obtained: trans isomer (50 mg, 31% yield) as a white solid, LC-MS (ES, m/z): 529[M+H]⁺; cis-isomer (50 mg, 31% yield) as a white solid, LC-MS (ES, m/z): 529[M+H]⁺.

### Step 4: Separation of Enantiomers to Obtain Example 24 and Example 25

50 mg of (trans)-N-(5-chloro-6-(2H-1,2,3-triazol-2-yl)pyridin-3-yl)-8-(1-(difluoromethyl)-1H-pyrazol-4-yl)-2-fluoro-8-methyl-7,8-dihydro-6H-cyclopenta[e]pyrazolo[1,5-a]pyrimidine-6-carboxamide were submitted to chiral HPLC purification (Column: CHIRALPAK ID, 2*25 cm, 5 μm; Mobile Phase A: Hex (0.1% FA)-HPLC, Mobile Phase B: EtOH-HPLC; Flow rate: 20 mL/min; Gradient: isocratic 40% Mobile Phase B; Wave Length: 220/254 nm; RT1 (min): 9.29; RT2 (min): 13.952; Sample Solvent: EtOH-HPLC; Injection Volume: 1.2 mL; Number Of Runs: 3). The second eluting isomer was concentrated and lyophilized to afford Example 24 (17.3 mg, 34% yield) as a white solid. The first eluting isomer was concentrated and lyophilized to afford Example 25 (14.5 mg, 29% yield) as a white solid.

Example 24: ¹H NMR (300 MHz, Chloroform-d) δ 8.56-8.70 (m, 4H), 7.93 (s, 2H), 7.86 (s, 1H), 7.68 (s, 1H), 7.12 (t, J=60.6 Hz, 1H), 6.33 (d, J=5.1 Hz, 1H), 4.39-4.48 (m, 1H), 3.02-3.06 (m, 1H), 2.73-2.79 (m, 1H), 2.13 (s, 3H). LC-MS (ES, m/z): 529[M+H]⁺.

Example 25: ¹H NMR (300 MHz, Chloroform-d) δ 8.69 (s, 1H), 8.46-8.62 (m, 2H), 8.14 (s, 1H), 7.94 (s, 2H), 7.83 (s, 1H), 7.66 (s, 1H), 7.13 (t, J=60.6 Hz, 1H), 6.32 (d, J=5.1 Hz, 1H), 4.32-4.42 (m, 1H), 3.00-3.05 (m, 1H), 2.73-2.77 (m, 1H), 2.13 (s, 3H). LC-MS (ES, m/z): 529[M+H]⁺.

### Step 5: Separation of Enantiomers to Obtain Example 26 and Example 27

50 mg of (cis)-N-(5-chloro-6-(2H-1,2,3-triazol-2-yl)pyridin-3-yl)-8-(1-(difluoromethyl)-1H-pyrazol-4-yl)-2-fluoro-8-methyl-7,8-dihydro-6H-cyclopenta[e]pyrazolo[1,5-a]pyrimidine-6-carboxamide were submitted to chiral HPLC purification (Column: CHIRALPAK IA, 2*25 cm, 5 m; Mobile Phase A: Hex (0.1% FA)-HPLC, Mobile Phase B: EtOH-HPLC; Flow rate: 20 mL/min; Gradient: isocratic 40% Mobile Phase B; Wave Length: 220/254 nm; RT1 (min): 9.026; RT2 (min): 15.277; Sample Solvent: EtOH-HPLC; Injection Volume: 1.5 mL; Number Of Runs: 2). The second eluting isomer was concentrated and lyophilized to afford Example 26 (10.6 mg, 21% yield) as a white solid. The first eluting isomer was concentrated and lyophilized to afford Example 27 (13.6 mg, 27% yield) as a white solid.

Example 26: ¹H NMR (300 MHz, Chloroform-d) δ 8.60 (s, 2H), 8.42 (s, 1H), 8.08 (s, 1H), 7.95 (s, 1H), 7.93 (s, 2H), 7.78 (s, 1H), 7.13 (t, J=60.3 Hz, 1H), 6.31 (d, J=5.1 Hz, 1H), 4.37-4.42 (m, 1H), 2.97-3.03 (m, 1H), 2.82-2.90 (m, 1H), 2.00 (s, 3H). LC-MS (ES, m/z): 529[M+H]⁺.

Example 27: ¹H NMR (300 MHz, Chloroform-d) δ 8.61 (s, 2H), 8.42 (s, 1H), 7.89-7.93 (m, 4H), 7.78 (s, 1H), 7.13 (t, J=60.6 Hz, 1H), 6.32 (d, J=4.8 Hz, 1H), 4.36-4.42 (m, 1H), 2.98-3.03 (m, 1H), 2.84-2.91 (m, 1H), 2.01 (s, 3H). LC-MS (ES, m/z): 529[M+H]⁺.

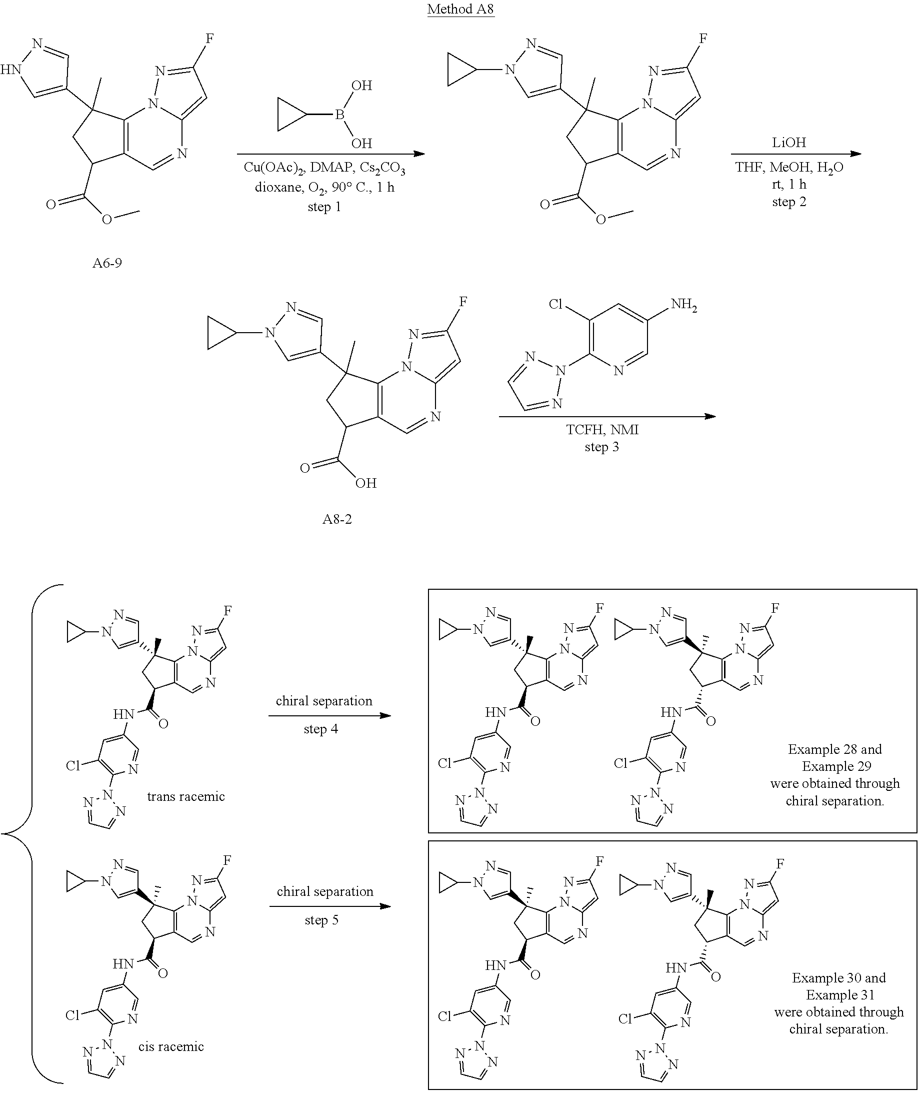

### Examples 28, 29: (6S,8R)—N-(5-chloro-6-(2H-1,2,3-triazol-2-yl)pyridin-3-yl)-8-(1-cyclopropyl-1H-pyrazol-4-yl)-2-fluoro-8-methyl-7,8-dihydro-6H-cyclopenta[e]pyrazolo[1,5-a]pyrimidine-6-carboxamide; (6R,8S)—N-(5-chloro-6-(2H-1,2,3-triazol-2-yl)pyridin-3-yl)-8-(1-cyclopropyl-1H-pyrazol-4-yl)-2-fluoro-8-methyl-7,8-dihydro-6H-cyclopenta[e]pyrazolo[1,5-a]pyrimidine-6-carboxamide

### Examples 30, 31: (6S,8S)—N-(5-chloro-6-(2H-1,2,3-triazol-2-yl)pyridin-3-yl)-8-(1-cyclopropyl-1H-pyrazol-4-yl)-2-fluoro-8-methyl-7,8-dihydro-6H-cyclopenta[e]pyrazolo[1,5-a]pyrimidine-6-carboxamide and (6R,8R)—N-(5-chloro-6-(2H-1,2,3-triazol-2-yl)pyridin-3-yl)-8-(1-cyclopropyl-1H-pyrazol-4-yl)-2-fluoro-8-methyl-7,8-dihydro-6H-cyclopenta[e]pyrazolo[1,5-a]pyrimidine-6-carboxamide

The relative and absolute stereochemistry of each example was not determined.

### Step 1: Methyl 8-(1-cyclopropyl-1H-pyrazol-4-yl)-2-fluoro-8-methyl-7,8-dihydro-6H-cyclopenta[e]pyrazolo[1,5-a]pyrimidine-6-carboxylate

To a stirred solution of methyl 2-fluoro-8-methyl-8-(1H-pyrazol-4-yl)-7,8-dihydro-6H-cyclopenta[e]pyrazolo[1,5-a]pyrimidine-6-carboxylate (A6-9, 250 mg, 793 mol) in dioxane (10 mL) were added cyclopropylboronic acid (136 mg, 1.6 mmol), DMAP (387 mg, 3.2 mmol), acetoxycopper (158 mg, 793 μmol) and cesium carbonate (646 mg, 2.0 mmol). The mixture was stirred at 90° C. for 1 h under oxygen atmosphere. The reaction was quenched with water (30 mL), and the resulting mixture was extracted with ethyl acetate (3×30 mL). The combined organic layers were dried over anhydrous sodium sulfate and concentrated under reduced pressure. The residue was applied on a silica gel column and eluted with EtOAc/PE (1:1) to afford the title compound (120 mg, 36% yield) as a yellow oil. LC-MS: m/z 356 [M+H]⁺.

### Step 2: 8-(1-cyclopropyl-1H-pyrazol-4-yl)-2-fluoro-8-methyl-7,8-dihydro-6H-cyclopenta[e]pyrazolo[1,5-a]pyrimidine-6-carboxylic acid (A8-2)

In analogy to Method A7 step 2 but using methyl 8-(1-cyclopropyl-1H-pyrazol-4-yl)-2-fluoro-8-methyl-7,8-dihydro-6H-cyclopenta[e]pyrazolo[1,5-a]pyrimidine-6-carboxylate (120 mg, 338 mol), the crude title compound was obtained as a yellow oil (A8-2, 100 mg). LC-MS: m/z 342 [M+H]⁺.

### Step 3: (trans)-N-(5-chloro-6-(2H-1,2,3-triazol-2-yl)pyridin-3-yl)-8-(1-cyclopropyl-1H-pyrazol-4-yl)-2-fluoro-8-methyl-7,8-dihydro-6H-cyclopenta[e]pyrazolo[1,5-a]pyrimidine-6-carboxamide and (cis)-N-(5-chloro-6-(2H-1,2,3-triazol-2-yl)pyridin-3-yl)-8-(1-cyclopropyl-1H-pyrazol-4-yl)-2-fluoro-8-methyl-7,8-dihydro-6H-cyclopenta[e]pyrazolo[1,5-a]pyrimidine-6-carboxamide

In analogy to Method A1 step 8 but using 8-(1-cyclopropyl-1H-pyrazol-4-yl)-2-fluoro-8-methyl-7,8-dihydro-6H-cyclopenta[e]pyrazolo[1,5-a]pyrimidine-6-carboxylic acid (100 mg, 293 μmol) and 5-chloro-6-(triazol-2-yl)pyridin-3-amine (57 mg, 293 μmol), racemic mixtures of the title compounds were obtained: trans isomer (20 mg, 13% yield) as a white solid, LC-MS: m/z 519 [M+H]⁺; cis-isomer (13 mg, 8% yield) as a white solid, LC-MS: m/z 519 [M+H]⁺.

### Step 4: Separation of Enantiomers to Obtain Example 28 and Example 29

20 mg of (trans)-N-(5-chloro-6-(2H-1,2,3-triazol-2-yl)pyridin-3-yl)-8-(1-cyclopropyl-1H-pyrazol-4-yl)-2-fluoro-8-methyl-7,8-dihydro-6H-cyclopenta[e]pyrazolo[1,5-a]pyrimidine-6-carboxamide were submitted to chiral HPLC purification. (Column: CHIRAL ART Amylose-SA, 2*25 cm, 5 m; Mobile Phase A: Hex (0.1% FA)-HPLC, Mobile Phase B: IPA-HPLC; Flow rate: 19 mL/min; Gradient: isocratic 50% Mobile Phase B; Wave Length: 220/254 nm; RT1 (min): 7.427; RT2 (min): 13.024; Sample Solvent: EtOH-HPLC; Injection Volume: 2.5 mL; Number Of Runs: 1). The second eluting isomer was concentrated and lyophilized to afford Example 28 (4.7 mg, 23% yield) as a white solid. The first eluting isomer was concentrated and lyophilized to afford Example 29 (5.5 mg, 27% yield) as a white solid.

Example 28: ¹H NMR (300 MHz, Chloroform-d) δ 9.13 (br, 1H), 8.71 (s, 1H), 8.61 (s, 1H), 8.53 (s, 1H), 7.93 (s, 2H), 7.73 (s, 1H), 6.29 (d, J=5.1 Hz, 1H), 4.45-4.50 (m, 1H), 3.52-3.60 (m, 111), 3.09-3.16 (m, 1H), 2.63-2.76 (m, 1H), 2.08 (s, 3H), 1.00-1.11 (m, 411). LC-MS: m/z 519 [M+H]⁺.

Example 29: ¹H NMR (300 MHz, Chloroform-d) δ 9.11 (br, 1H), 8.71 (s, 1H), 8.60 (s, 1H), 8.52 (s, 1H), 7.93 (s, 2H), 7.61-7.71 (m, 2H), 6.29 (d, J=4.8 Hz, 1H), 4.43-4.49 (m, 1H), 3.51-3.57 (m, 1H), 3.07-3.14 (m, 1H), 2.63-2.70 (m, 1H), 2.08 (s, 3H), 1.00-1.10 (m, 4H). LC-MS: m/z 519 [M+H]⁺.

### Step 5: Separation of Enantiomers to Obtain Example 30 and Example 31

13 mg of (cis)-N-(5-chloro-6-(2H-1,2,3-triazol-2-yl)pyridin-3-yl)-8-(1-cyclopropyl-11H-pyrazol-4-yl)-2-fluoro-8-methyl-7,8-dihydro-6H-cyclopenta[e]pyrazolo[1,5-a]pyrimidine-6-carboxamide were submitted to chiral HPLC purification. (Column: CHIRAL ART Amylose-SA, 2*25 cm, 5 m; Mobile Phase A: Hex (0.1% FA)-HPLC, Mobile Phase B: IPA-HPLC; Flow rate: 19 mL/min; Gradient: isocratic 50% Mobile Phase B; Wave Length: 220/254 nm; RT1 (min): 5.137; RT2 (min): 8.122; Sample Solvent: EtOH-HPLC; Injection Volume: 1.8 mL; Number Of Runs: 2). The second eluting isomer was concentrated and lyophilized to afford Example 30 (2.1 mg, 16% yield) as a white solid. The first eluting isomer was concentrated and lyophilized to afford Example 31 (2.5 mg, 18% yield) as a white solid.

Example 30: ¹H NMR (300 MHz, Chloroform-d) δ 8.59 (s, 1H), 8.51 (s, 1H), 8.30-8.42 (m, 2H), 7.92 (s, 2H), 7.59-7.66 (m, 2H), 6.28 (d, J=5.1 Hz, 1H), 4.39-4.45 (m, 1H), 3.52-3.58 (m, 1H), 2.82-3.03 (m, 2H), 2.01 (s, 3H), 0.99-1.12 (m, 4H). LC-MS: m/z 519 [M+H]⁺.

Example 31: ¹H NMR (300 MHz, Chloroform-d) δ 8.59 (s, 1H), 8.50 (s, 1H), 8.39 (s, 1H), 8.13 (s, 1H), 7.93 (s, 2H), 7.56-7.64 (m, 2H), 6.30 (d, J=5.1 Hz, 114), 4.38-4.45 (m, 1H), 3.51-3.56 (m, 1H), 2.85-3.02 (m, 2H), 2.01 (s, 3H), 0.93-1.09 (m, 4H). LC-MS: m/z 519 [M+H]⁺.

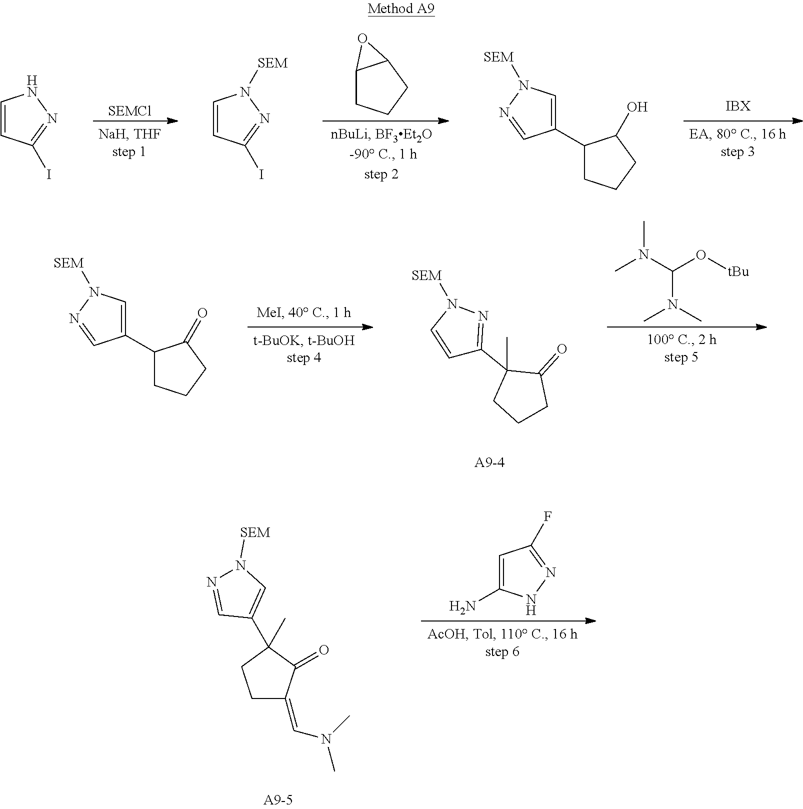

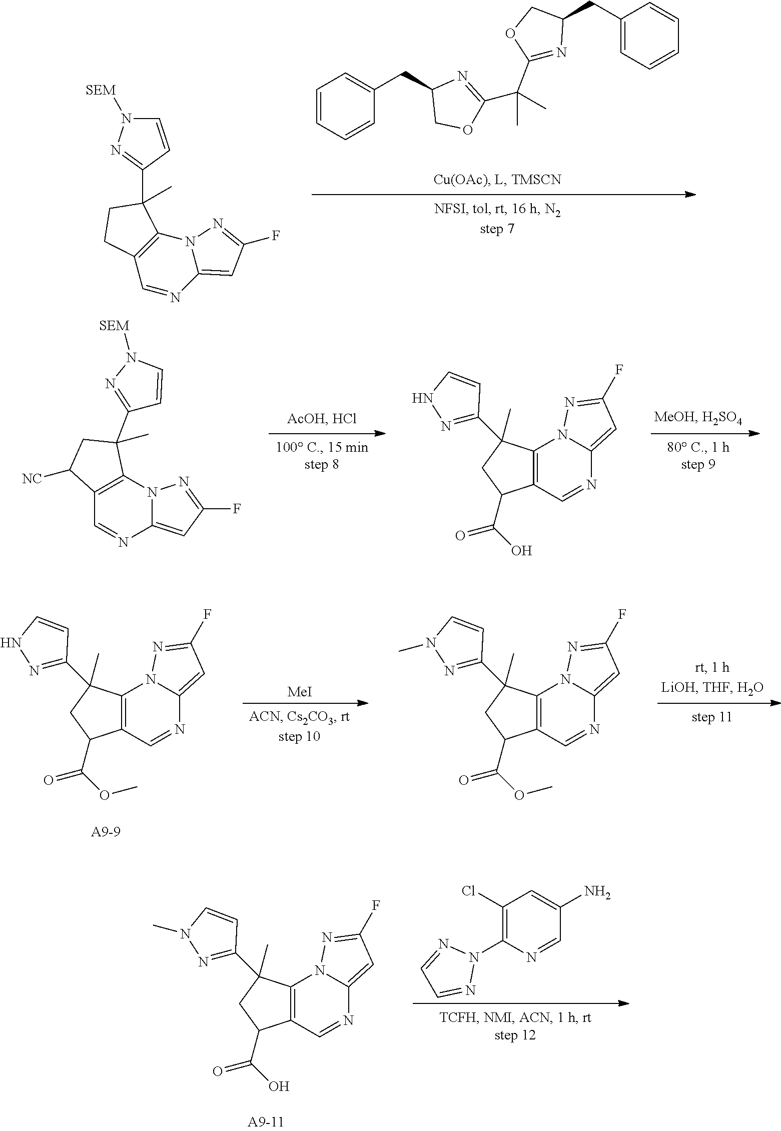

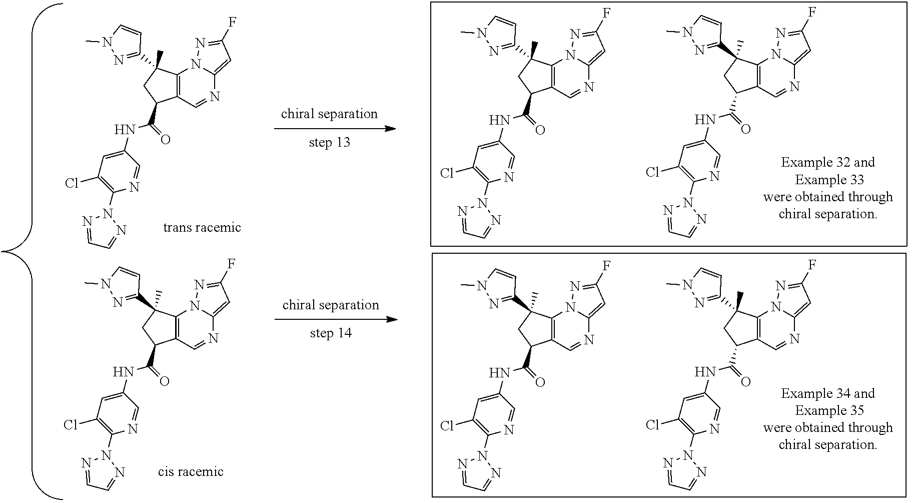

### Examples 32, 33: (6S,8S)—N-(5-chloro-6-(2H-1,2,3-triazol-2-yl)pyridin-3-yl)-2-fluoro-8-methyl-8-(1-methyl-1H-pyrazol-3-yl)-7,8-dihydro-6H-cyclopenta[e]pyrazolo[1,5-a]pyrimidine-6-carboxamide; (6R,8R)—N-(5-chloro-6-(2H-1,2,3-triazol-2-yl)pyridin-3-yl)-2-fluoro-8-methyl-8-(1-methyl-1H-pyrazol-3-yl)-7,8-dihydro-6H-cyclopenta[e]pyrazolo[1,5-a]pyrimidine-6-carboxamide

### Examples 34, 35: (6S,8R)—N-(5-chloro-6-(2H-1,2,3-triazol-2-yl)pyridin-3-yl)-2-fluoro-8-methyl-8-(1-methyl-1H-pyrazol-3-yl)-7,8-dihydro-6H-cyclopenta[e]pyrazolo[1,5-a]pyrimidine-6-carboxamide; (6R,8S)—N-(5-chloro-6-(2H-1,2,3-triazol-2-yl)pyridin-3-yl)-2-fluoro-8-methyl-8-(1-methyl-1H-pyrazol-3-yl)-7,8-dihydro-6H-cyclopenta[e]pyrazolo[1,5-a]pyrimidine-6-carboxamide

The relative and absolute stereochemistry of each example was not determined.

### Step 1: 3-iodo-1-((2-(trimethylsilyl)ethoxy)methyl)-1H-pyrazole

In analogy to Method A6 step 1 but using 3-iodo-1H-pyrazole (100 g, 515.5 mmol), the title compound was obtained as a white solid (130 g, 74% yield). LC-MS: m/z 325 [M+H]⁺.

### Step 2: 2-(1-((2-(trimethylsilyl)ethoxy)methyl)-1H-pyrazol-3-yl)cyclopentan-1-ol

To a stirred mixture of 3-iodo-1-((2-(trimethylsilyl)ethoxy)methyl)-1H-pyrazole (231.3 g, 713.3 mmol) in Et₂O (4.5 L) was added n-Butyllithium (310 mL, 2.5M in hexane) at −90° C. under nitrogen. The reaction mixture was stirred at −90° C. for 30 min under nitrogen. 6-oxabicyclo[3.1.0]hexane (50 g, 594.4 mmol) and boron trifluoride diethyl etherate (109 g, 772.7 mmol) were added at −90° C. The reaction mixture was stirred at −90° C. for 1 h under nitrogen. The reaction mixture was quenched with saturated aqueous ammonium chloride (1000 mL), and the resulting mixture was extracted with ethyl acetate (3×2.0 L). The combined organic layers were dried over anhydrous sodium sulfate and concentrated under reduced pressure. The residue was applied onto a silica gel column and eluted with EtOAc/PE (1:1) to afford the title compound (60 g, 35% yield) as a yellow oil. LC-MS: m/z 283 [M+H]⁺.

### Step 3: 2-(1-((2-(trimethylsilyl)ethoxy)methyl)-1H-pyrazol-3-yl)cyclopentan-1-one

In analogy to Method A1 step 2 but using 2-(1-((2-(trimethylsilyl)ethoxy)methyl)-1H-pyrazol-3-yl)cyclopentan-1-ol (60 g, 212.4 mmol), the title compound was obtained as a yellow oil (30 g, 31% purity). LC-MS: m/z 281 [M+H]⁺.

### Step 4: 2-methyl-2-(1-((2-(trimethylsilyl)ethoxy)methyl)-1H-pyrazol-3-yl)cyclopentan-1-one (A9-4)

In analogy to Method A1 step 3 but using 2-(1-((2-(trimethylsilyl)ethoxy)methyl)-1H-pyrazol-4-yl)cyclopentan-1-one (23 g, 49.2 mmol) and after purification of the product by prep-HPLC, the title compound was obtained as a yellow oil (A9-4, 4.7 g, 32% yield). LC-MS: m/z 295 [M+H]⁺.

### Step 5: 5-((dimethylamino)methylene)-2-methyl-2-(1-((2-(trimethylsilyl)ethoxy)methyl)-1H-pyrazol-3-yl)cyclopentan-1-one (A9-5)

In analogy to Method A1 step 4 but using 2-methyl-2-(1-((2-(trimethylsilyl)ethoxy)methyl)-1H-pyrazol-3-yl)cyclopentan-1-one (4.7 g, 15.9 mmol) in 1-tert-butoxy-N,N,N′,N′-tetramethyl-methanediamine (8.3 g, 47.7 mmol) and stirring at 100° C. for 2 h, the crude title compound was obtained as a yellow oil (A9-5, 4.7 g) and was used in the next step without further purification. LC-MS: m/z 350 [M+H]⁺.

### Step 6: 2-fluoro-8-methyl-8-(1-((2-(trimethylsilyl)ethoxy)methyl)-1H-pyrazol-3-yl)-7,8-dihydro-6H-cyclopenta[e]pyrazolo[1,5-a]pyrimidine

In analogy to Method A6 step 6 but using 5-((dimethylamino)methylene)-2-methyl-2-(1-((2-(trimethylsilyl)ethoxy)methyl)-1H-pyrazol-3-yl)cyclopentan-1-one (4 g, 11.4 mmol) and stirring at 110° C. for 16 h, the title compound was obtained as a yellow oil (2 g, 45% yield). LC-MS: m/z 388 [M+H]⁺.

### Step 7: 2-fluoro-8-methyl-8-(1-((2-(trimethylsilyl)ethoxy)methyl)-1H-pyrazol-3-yl)-7,8-dihydro-6H-cyclopenta[e]pyrazolo[1,5-a]pyrimidine-6-carbonitrile

In analogy to Method A6 step 7 but using 2-fluoro-8-methyl-8-(1-((2-(trimethylsilyl)ethoxy)methyl)-1H-pyrazol-3-yl)-7,8-dihydro-6H-cyclopenta[e]pyrazolo[1,5-a]pyrimidine (2 g, 5.1 mmol) and stirring at 25° C. for 24 h, the title compound was obtained as a yellow oil (420 mg, 11% yield) as a yellow oil. LC-MS: m/z 413 [M+H]⁺.

### Step 8: 2-fluoro-8-methyl-8-(1H-pyrazol-3-yl)-7,8-dihydro-6H-cyclopenta[e]pyrazolo[1,5-a]pyrimidine-6-carboxylic acid

A solution of 2-fluoro-8-methyl-8-(1-((2-(trimethylsilyl)ethoxy)methyl)-1H-pyrazol-3-yl)-7,8-dihydro-6H-cyclopenta[e]pyrazolo[1,5-a]pyrimidine-6-carbonitrile (700 mg, 1.7 mmol) in AcOH (2.5 mL) and HCl (12M, 2.5 mL) was stirred at 100° C. for 15 min. The mixture was allowed to cool to 25° C. The reaction mixture was diluted with water (10 mL) and the pH was adjusted to ˜7 with saturated aqueous sodium bicarbonate solution. The resulting mixture was extracted with ethyl acetate (3×15 mL). The combined organic layers were washed with brine (30 mL), dried over anhydrous sodium sulfate and concentrated under reduced pressure. The mixture was purified by prep-HPLC to give the title compound (160 mg, 26% yield) as a yellow oil. LC-MS: m/z 302 [M+H]⁺.

### Step 9: Methyl 2-fluoro-8-methyl-8-(1H-pyrazol-3-yl)-7,8-dihydro-6H-cyclopenta[e]pyrazolo[1,5-a]pyrimidine-6-carboxylate (A9-9)

In analogy to Method A6 step 9 but using 2-fluoro-8-methyl-8-(1H-pyrazol-3-yl)-7,8-dihydro-6H-cyclopenta[e]pyrazolo[1,5-a]pyrimidine-6-carboxylic acid (330 mg, 33.2 mmol), the crude title compound was obtained as a yellow oil (A9-9, 100 mg). LC-MS: m/z 316 [M+H]⁺.

### Step 10: Methyl 2-fluoro-8-methyl-8-(1-methyl-1H-pyrazol-3-yl)-7,8-dihydro-6H-cyclopenta[e]pyrazolo[1,5-a]pyrimidine-6-carboxylate

In analogy to Method A6 step 10 but using crude methyl 2-fluoro-8-methyl-8-(1H-pyrazol-3-yl)-7,8-dihydro-6H-cyclopenta[e]pyrazolo[1,5-a]pyrimidine-6-carboxylate (80 mg) and without final purification, the crude title compound was obtained as a yellow oil (50 mg). 11H NMR (400 MHz, DMSO-d₆) δ 8.67 (s, 1H), 7.55 (d, J=2.4 Hz, 1H), 6.52 (d, J=5.2 Hz, 1H), 6.14 (d, J=2.0 Hz, 1H), 4.30-4.46 (m, 1H), 3.68 (s, 3H), 3.67 (s, 3H), 2.83-2.88 (m, 1H), 2.60-2.66 (m, 1H), 1.86 (s, 3H). LC-MS: m/z 330 [M+H]⁺.

### Step 11: 2-fluoro-8-methyl-8-(1-methyl-1H-pyrazol-3-yl)-7,8-dihydro-6H-cyclopenta[e]pyrazolo[1,5-a]pyrimidine-6-carboxylic acid (A9-11)

In analogy to Method A6 step 11 but using crude methyl 2-fluoro-8-methyl-8-(1-methyl-1H-pyrazol-3-yl)-7,8-dihydro-6H-cyclopenta[e]pyrazolo[1,5-a]pyrimidine-6-carboxylate (50 mg, 142 mol), the crude title compound was obtained as a yellow oil (A9-11, 40 mg). LC-MS: m/z 316 [M+H]⁺.

### Step 12: (trans)-N-(5-chloro-6-(2H-1,2,3-triazol-2-yl)pyridin-3-yl)-2-fluoro-8-methyl-8-(1-methyl-1H-pyrazol-3-yl)-7,8-dihydro-6H-cyclopenta[e]pyrazolo[1,5-a]pyrimidine-6-carboxamide and (cis)-N-(5-chloro-6-(2H-1,2,3-triazol-2-yl)pyridin-3-yl)-2-fluoro-8-methyl-8-(1-methyl-1H-pyrazol-3-yl)-7,8-dihydro-6H-cyclopenta[e]pyrazolo[1,5-a]pyrimidine-6-carboxamide

In analogy to Method A1 step 8 but using 2-fluoro-8-methyl-8-(1-methyl-1H-pyrazol-3-yl)-7,8-dihydro-6H-cyclopenta[e]pyrazolo[1,5-a]pyrimidine-6-carboxylic acid (A9-11, 40 mg, 127 μmol) and 5-chloro-6-(triazol-2-yl)pyridin-3-amine (24 mg, 127 μmol), racemic mixtures of the title compounds were obtained: trans isomer (23 mg, 37% yield) as a white solid, LC-MS: m/z 493 [M+H]⁺; cis-isomer (14 mg, 22% yield) as a white solid, LC-MS: m/z 493 [M+H]⁺.

### Step 13: Separation of Enantiomers to Obtain Example 32 and Example 33

23 mg of (trans)-N-(5-chloro-6-(2H-1,2,3-triazol-2-yl)pyridin-3-yl)-2-fluoro-8-methyl-8-(1-methyl-1H-pyrazol-3-yl)-7,8-dihydro-6H-cyclopenta[e]pyrazolo[1,5-a]pyrimidine-6-carboxamide were submitted to chiral HPLC purification. (Column: CHIRAL ART Amylose-SA, 2*25 cm, 5 m; Mobile Phase A: Hex (0.1% FA)-HPLC, Mobile Phase B: EtOH-HPLC; Flow rate: 25 mL/min; Gradient: isocratic 30% Mobile Phase B; Wave Length: 220/254 nm; RT1 (min): 28.444; RT2 (min): 33.535; Sample Solvent: EtOH-HPLC; Injection Volume: 0.5 mL; Number Of Runs: 12). The second eluting isomer was concentrated and lyophilized to afford Example 32 (4 mg, 20% yield) as a white solid. The first eluting isomer was concentrated and lyophilized to afford Example 33 (2.9 mg, 14% yield) as a white solid.

Example 32: ¹H NMR (400 MHz, DMSO-d₆) δ:11.14 (s, 1H), 8.76 (d, J=2.0 Hz, 1H), 8.68 (s, 1H), 8.61 (d, J=2.0 Hz, 1H), 8.18 (s, 2H), 7.62 (d, J=2.0 Hz, 1H), 6.55 (d, J=5.2 Hz, 1H), 6.28 (d, J=2.0 Hz, 1H), 4.51-4.56 (m, 1H), 3.73 (s, 3H), 3.00-3.33 (m, 1H), 2.50-2.51 (m, 1H), 2.00 (s, 3H). LC-MS: m/z 493 [M+H]⁺.

Example 33: ¹H NMR (400 MHz, DMSO-d₆) δ:11.14 (br), 8.75 (d, J=2.4 Hz, 1H), 8.68 (s, 1H), 8.61 (d, J=2.0 Hz, 1H), 8.18 (s, 2H), 7.62 (d, J=2.0 Hz, 1H), 6.55 (d, J=4.8 Hz, 1H), 6.28 (d, J=2.0 Hz, 1H), 4.52-4.56 (m, 1H), 3.73 (s, 3H), 3.00-3.33 (m, 1H), 2.47-2.50 (m, 1H), 2.00 (s, 3H). LC-MS: m/z 493 [M+H]⁺.

### Step 14: Separation of Enantiomers to Obtain Example 34 and Example 35

14 mg of (cis)-N-(5-chloro-6-(2H-1,2,3-triazol-2-yl)pyridin-3-yl)-2-fluoro-8-methyl-8-(1-methyl-1H-pyrazol-3-yl)-7,8-dihydro-6H-cyclopenta[e]pyrazolo[1,5-a]pyrimidine-6-carboxamide was submitted to chiral HPLC purification. (Column: CHIRALPAK AS-H, 2*25 cm, 5 μm; Mobile Phase A: Hex (0.1% FA)-HPLC, Mobile Phase B: EtOH-HPLC; Flow rate: 20 mL/min; Gradient: isocratic 30% Mobile Phase B; Wave Length: 220/254 nm; RT1 (min): 9.135; RT2 (min): 20.036; Sample Solvent: EtOH-HPLC; Injection Volume: 1.6 mL; Number Of Runs: 2). The first eluting isomer was concentrated and lyophilized to afford Example 34 (2.0 mg, 14% yield) as a white solid. The second eluting isomer was concentrated and lyophilized to afford Example 35 (1.7 mg, 12% yield) as a white solid.

Example 34: ¹H NMR (400 MHz, DMSO-d₆) δ:11.04 (s, 1H), 8.73 (d, J=2.4 Hz, 1H), 8.69 (s, 1H), 8.57 (d, J=2.4 Hz, 1H), 8.18 (s, 2H), 7.56 (d, J=2.4 Hz, 1H), 6.54 (d, J=4.8 Hz, 1H), 6.17 (d, J=2.4 Hz, 1H), 4.51-4.55 (m, 1H), 3.69 (s, 3H), 2.88-2.92 (m, 1H), 2.74-2.79 (m, 1H), 1.87 (s, 3H). LC-MS: m/z 493 [M+H]⁺.

Example 35: ¹H NMR (400 MHz, DMSO-d₆) δ:11.02 (s, 1H), 8.72 (d, J=2.0 Hz, 1H), 8.69 (s, 1H), 8.57 (d, J=2.4 Hz, 1H), 8.18 (s, 2H), 7.56 (d, J=2.4 Hz, 1H), 6.54 (d, J=4.8 Hz, 1H), 6.17 (d, J=2.0 Hz, 1H), 4.51-4.55 (m, 1H), 3.69 (s, 3H), 2.88-2.93 (m, 1H), 2.74-2.79 (m, 1H), 1.87 (s, 3H). LC-MS: m/z 493 [M+H]⁺.

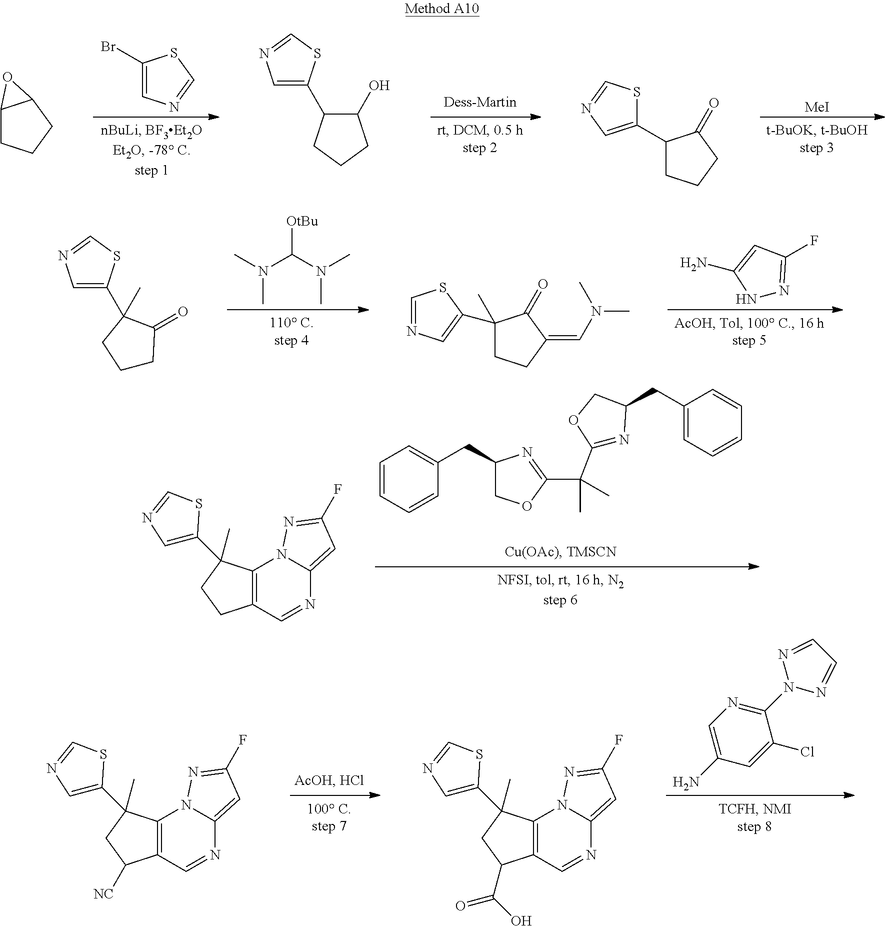

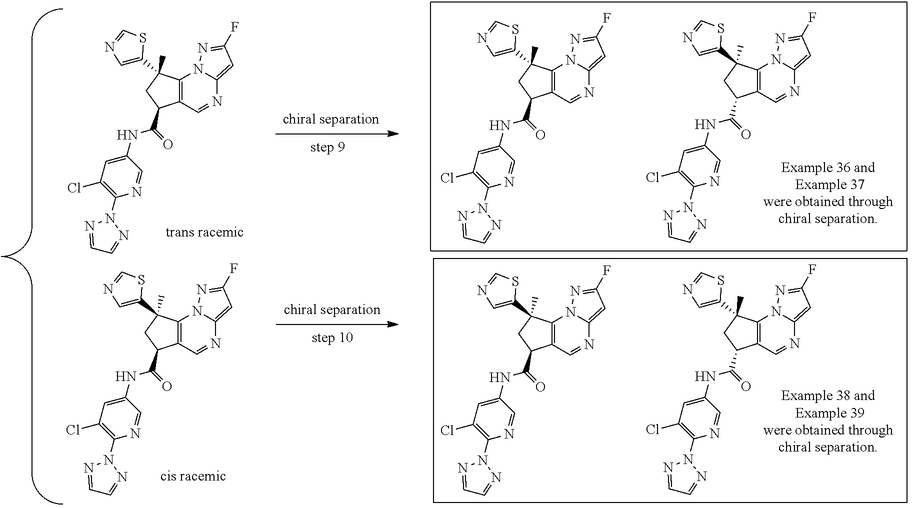

### Examples 36, 37: (6S,8S)—N-(5-chloro-6-(2H-1,2,3-triazol-2-yl)pyridin-3-yl)-2-fluoro-8-methyl-8-(thiazol-5-yl)-7,8-dihydro-6H-cyclopenta[e]pyrazolo[1,5-a]pyrimidine-6-carboxamide; (6R,8R)—N-(5-chloro-6-(2H-1,2,3-triazol-2-yl)pyridin-3-yl)-2-fluoro-8-methyl-8-(thiazol-5-yl)-7,8-dihydro-6H-cyclopenta[e]pyrazolo[1,5-a]pyrimidine-6-carboxamide

### Examples 38, 39: (6S,8R)—N-(5-chloro-6-(2H-1,2,3-triazol-2-yl)pyridin-3-yl)-2-fluoro-8-methyl-8-(thiazol-5-yl)-7,8-dihydro-6H-cyclopenta[e]pyrazolo[1,5-a]pyrimidine-6-carboxamide and (6R,8S)—N-(5-chloro-6-(2H-1,2,3-triazol-2-yl)pyridin-3-yl)-2-fluoro-8-methyl-8-(thiazol-5-yl)-7,8-dihydro-6H-cyclopenta[e]pyrazolo[1,5-a]pyrimidine-6-carboxamide

The relative and absolute stereochemistry of each example was not determined.

### Step 1: 2-(thiazol-5-yl)cyclopentan-1-ol

In analogy to Method A1 step 1 but using 5-bromothiazole (11.65 g, 71 mmol), the title compound was obtained as a brown oil (2 g, 20% yield). LC-MS: m/z 170 [M+H]⁺.

### Step 2: 2-(thiazol-5-yl)cyclopentan-1-one

To a stirred solution of 2-(thiazol-5-yl)cyclopentan-1-ol (1 g, 5.9 mmol) in DCM (100 mL) was added Dess-Martin periodinane (3.76 g, 8.8 mmol) at 25° C. The resulting mixture was stirred at 25° C. for 0.5 h. The reaction was quenched with saturated aqueous NaHSO₃ (200 mL), and the resulting mixture was extracted with DCM (3×100 mL). The combined organic layers were washed with saturated aqueous sodium bicarbonate solution (200 mL), dried over anhydrous sodium sulfate and concentrated under reduced pressure to give the crude title compound (1 g) as a yellow oil. LC-MS: m/z 168 [M+H]⁺.

### Step 3: 2-methyl-2-(thiazol-5-yl)cyclopentan-1-one

In analogy to Method A1 step 3 but using 2-(thiazol-5-yl)cyclopentan-1-one (1.5 g, 5.3 mmol) and purification of the product by silica gel column chromatography eluting with PE/EA (1:1), the title compound was obtained as a yellow oil (270 mg, 28% yield). LC-MS: m/z 182 [M+H]⁺.

### Step 4: 5-((dimethylamino)methylene)-2-methyl-2-(thiazol-5-yl)cyclopentan-1-one

In analogy to Method A1 step 4 but using 2-methyl-2-(thiazol-5-yl)cyclopentan-1-one (530 mg, 2.9 mmol) in 1-tert-butoxy-N,N,N′,N′-tetramethyl-methanediamine (8 mL) and stirring at 110° C. for 1.5 h, the crude title compound was obtained as a yellow oil (230 mg, crude). LC-MS: m/z 237 [M+H]⁺.

### Step 5: 5-(2-fluoro-8-methyl-7,8-dihydro-6H-cyclopenta[e]pyrazolo[1,5-a]pyrimidin-8-yl)thiazole

To a stirred solution of 5-((dimethylamino)methylene)-2-methyl-2-(thiazol-5-yl)cyclopentan-1-one (520 mg, 2.2 mmol) in toluene (10 mL) were added 5-fluoro-1H-pyrazol-3-amine (445 mg, 4.4 mmol) and AcOH (1 mL) at room temperature. The resulting mixture was stirred at 100° C. for 16 h. The mixture was allowed to cool to 25° C. The reaction mixture was concentrated under reduced pressure, and the residue was diluted with water (50 mL). The pH was adjusted to ˜7 with saturated aqueous sodium bicarbonate solution. The resulting mixture was extracted with ethyl acetate (2×60 mL). The combined organic layers were dried over anhydrous sodium sulfate and concentrated under reduced pressure. The residue was applied on a silica gel column chromatography and eluted with EA/PE (3:7) to give the title compound (430 mg, 71% yield) as a yellow solid. LC-MS: m/z 275 [M+H]⁺.

### Step 6: 2-fluoro-8-methyl-8-(thiazol-5-yl)-7,8-dihydro-6H-cyclopenta[e]pyrazolo[1,5-a]pyrimidine-6-carbonitrile

In analogy to Method A1 step 6 but using 5-(2-fluoro-8-methyl-7,8-dihydro-6H-cyclopenta[e]pyrazolo[1,5-a]pyrimidin-8-yl)thiazole (430 mg, 1.6 mmol), the title compound was obtained as a white solid (70 mg, 15% yield). LC-MS: m/z 300 [M+H]⁺.

### Step 7: 2-fluoro-8-methyl-8-(thiazol-5-yl)-7,8-dihydro-6H-cyclopenta[e]pyrazolo[1,5-a]pyrimidine-6-carboxylic acid

In analogy to Method A1 step 7 but using 2-fluoro-8-methyl-8-(thiazol-5-yl)-7,8-dihydro-6H-cyclopenta[e]pyrazolo[1,5-a]pyrimidine-6-carbonitrile (70 mg, 234 μmol) and stirring at 90° C. for 1 h, the title compound was obtained as a white solid (55 mg, 74% yield). LC-MS: m/z 319 [M+H]⁺.

### Step 8: (trans)-N-(5-chloro-6-(2H-1,2,3-triazol-2-yl)pyridin-3-yl)-2-fluoro-8-methyl-8-(thiazol-5-yl)-7,8-dihydro-6H-cyclopenta[e]pyrazolo[1,5-a]pyrimidine-6-carboxamide and (cis)-N-(5-chloro-6-(2H-1,2,3-triazol-2-yl)pyridin-3-yl)-2-fluoro-8-methyl-8-(thiazol-5-yl)-7,8-dihydro-6H-cyclopenta[e]pyrazolo[1,5-a]pyrimidine-6-carboxamide

In analogy to Method A1 step 8 but using 2-fluoro-8-methyl-8-(thiazol-5-yl)-7,8-dihydro-6H-cyclopenta[e]pyrazolo[1,5-a]pyrimidine-6-carboxylic acid (50 mg, 157 μmol) and 5-chloro-6-(2H-1,2,3-triazol-2-yl)pyridin-3-amine (31 mg, 157 μmol), racemic mixtures of the title compounds were obtained: trans isomer (35 mg, 44% yield) as an off-white solid, LC-MS: m/z 496 [M+H]⁺; cis-isomer (25 mg, 31% yield) as a white solid, LC-MS: m/z 496 [M+H]⁺.

### Step 9: Separation of Enantiomers to Obtain Example 36 and Example 37

35 mg of (trans)-N-(5-chloro-6-(2H-1,2,3-triazol-2-yl)pyridin-3-yl)-2-fluoro-8-methyl-8-(thiazol-5-yl)-7,8-dihydro-6H-cyclopenta[e]pyrazolo[1,5-a]pyrimidine-6-carboxamide were submitted to chiral HPLC purification. (Column: CHIRALPAK IF, 2*25 cm, 5 m; Mobile Phase A: Hex (0.1% FA)-HPLC, Mobile Phase B: IPA-HPLC; Flow rate: 20 mL/min; Gradient: isocratic 50% Mobile Phase B; Wave Length: 220/254 nm; RT1 (min): 19.03; RT2 (min): 41.07; Sample Solvent: Hex (0.1% FA)-HPLC; Injection Volume: 1.1 mL; Number Of Runs: 20). The second eluting isomer was concentrated and lyophilized to afford Example 36 (5 mg, 28% yield) as a white solid. The first eluting isomer was concentrated and lyophilized to afford Example 37 (6.7 mg, 38% yield) as a white solid.

Example 36: ¹HNMR (300 MHz, DMSO-d₆) δ:11.19 (br, 1H), 9.03 (s, 1H), 8.75 (s, 1H), 8.73 (d, J=2.1 Hz, 1H), 8.61 (d, J=2.1 Hz, 1H), 8.18 (s, 2H), 7.84 (s, 1H), 6.63 (d, J=5.1 Hz, 1H), 4.58-4.65 (m, 1H), 2.96-3.01 (m, 1H), 2.68-2.80 (m, 1H), 2.12 (s, 3H). LC-MS: m/z 496 [M+H]⁺.

Example 37: ¹HNMR (300 MHz, DMSO-d₆) δ: 11.20 (br, 1H), 9.03 (s, 1H), 8.75 (s, 1H), 8.73 (d, J=2.1 Hz, 1H), 8.61 (d, J=2.1 Hz, 1H), 8.18 (s, 2H), 7.84 (s, 1H), 6.63 (d, J=5.1 Hz, 1H), 4.58-4.65 (m, 1H), 2.96-3.01 (m, 1H), 2.68-2.78 (m, 1H), 2.12 (s, 3H). LC-MS: m/z 496 [M+H]⁺.

### Step 10: Separation of Enantiomers to Obtain Example 38 and Example 39

25 mg of (cis)-N-(5-chloro-6-(2H-1,2,3-triazol-2-yl)pyridin-3-yl)-2-fluoro-8-methyl-8-(thiazol-5-yl)-7,8-dihydro-6H-cyclopenta[e]pyrazolo[1,5-a]pyrimidine-6-carboxamide were submitted to chiral HPLC purification. (Column: CHIRAL ART Amylose-SA, 2*25 cm, 5 μm; Mobile Phase A: Hex (0.1% FA)-HPLC, Mobile Phase B: EtOH-HPLC; Flow rate: 20 mL/min; Gradient: isocratic 50% Mobile Phase B; Wave Length: 220/254 nm; RT1 (min): 10.14; RT2 (min): 12.47; Sample Solvent: EtOH-HPLC; Injection Volume: 0.4 mL; Number Of Runs: 3). The first eluting isomer was concentrated and lyophilized to afford Example 38 (4.2 mg, 33% yield) as a white solid. The second eluting isomer was concentrated and lyophilized to afford Example 39 (4.7 mg, 37% yield) as a white solid.

Example 38: ¹HNMR (300 MHz, DMSO-d₆) δ:11.22 (br, 1H), 8.99 (s, 1H), 8.77 (s, 1H), 8.73 (d, J=2.1 Hz, 1H), 8.58 (d, J=2.1 Hz, 1H), 8.18 (s, 2H), 7.82 (s, 1H), 6.61 (d, J=4.8 Hz, 1H), 4.58-4.64 (m, 1H), 2.95-3.02 (m, 1H), 2.74-2.81 (m, 1H), 2.03 (s, 3H). LC-MS: m/z 496 [M+H]⁺.

Example 39: ¹HNMR (300 MHz, DMSO-d₆) δ:11.22 (br, 1H), 8.99 (s, 1H), 8.78 (s, 1H), 8.73 (d, J=2.4 Hz, 1H), 8.57 (d, J=2.4 Hz, 1H), 8.17 (s, 2H), 7.82 (s, 1H), 6.61 (d, J=5.1 Hz, 1H), 4.58-4.64 (m, 1H), 2.95-3.01 (m, 1H), 2.74-2.82 (m, 1H), 2.03 (s, 3H). LC-MS: m/z 496 [M+H]⁺.

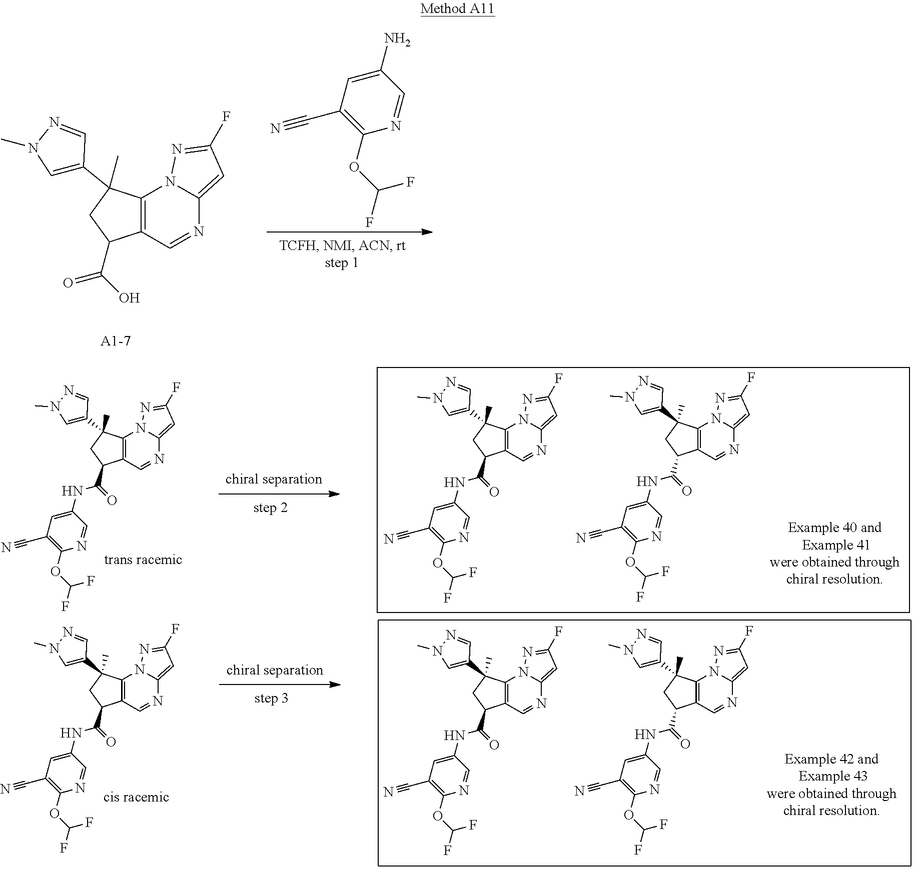

### Examples 40, 41: (6S,8R)—N-(5-cyano-6-(difluoromethoxy)pyridin-3-yl)-2-fluoro-8-methyl-8-(1-methyl-1H-pyrazol-4-yl)-7,8-dihydro-6H-cyclopenta[e]pyrazolo[1,5-a]pyrimidine-6-carboxamide; (6R,8S)—N-(5-cyano-6-(difluoromethoxy)pyridin-3-yl)-2-fluoro-8-methyl-8-(1-methyl-1H-pyrazol-4-yl)-7,8-dihydro-6H-cyclopenta[e]pyrazolo[1,5-a]pyrimidine-6-carboxamide

### Examples 42, 43: (6S,8S)—N-(5-cyano-6-(difluoromethoxy)pyridin-3-yl)-2-fluoro-8-methyl-8-(1-methyl-1H-pyrazol-4-yl)-7,8-dihydro-6H-cyclopenta[e]pyrazolo[1,5-a]pyrimidine-6-carboxamide; (6R,8R)—N-(5-cyano-6-(difluoromethoxy)pyridin-3-yl)-2-fluoro-8-methyl-8-(1-methyl-1H-pyrazol-4-yl)-7,8-dihydro-6H-cyclopenta[e]pyrazolo[1,5-a]pyrimidine-6-carboxamide

The relative and absolute stereochemistry of each example was not determined.

### Step 1: Trans-N-(5-cyano-6-(difluoromethoxy)pyridin-3-yl)-2-fluoro-8-methyl-8-(1-methyl-1H-pyrazol-4-yl)-7,8-dihydro-6H-cyclopenta[e]pyrazolo[1,5-a]pyrimidine-6-carboxamide and cis-N-(5-cyano-6-(difluoromethoxy)pyridin-3-yl)-2-fluoro-8-methyl-8-(1-methyl-1H-pyrazol-4-yl)-7,8-dihydro-6H-cyclopenta[e]pyrazolo[1,5-a]pyrimidine-6-carboxamide

In analogy to Method A1 step 8 but using 2-fluoro-8-methyl-8-(1-methyl-1H-pyrazol-4-yl)-7,8-dihydro-6H-cyclopenta[e]pyrazolo[1,5-a]pyrimidine-6-carboxylic acid (90 mg, 285 μmol) and 5-amino-2-(difluoromethoxy)nicotinonitrile (53 mg, 285 μmol), racemic mixtures of the title compounds were obtained: trans isomer (18 mg, 13% yield) as a white solid, LC-MS: m/z 483 [M+H]⁺; cis-isomer (12.4 mg, 9% yield) as a white solid, LC-MS: m/z 483 [M+H]⁺.

### Step 2: Separation of Enantiomers to Obtain Example 40 and Example 41

18 mg of trans-N-(5-cyano-6-(difluoromethoxy)pyridin-3-yl)-2-fluoro-8-methyl-8-(1-methyl-1H-pyrazol-4-yl)-7,8-dihydro-6H-cyclopenta[e]pyrazolo[1,5-a]pyrimidine-6-carboxamide were submitted to chiral HPLC purification (Column: CHIRALPAK IF, 2*25 cm, 5 m; Mobile Phase A: Hex (0.1% FA)-HPLC, Mobile Phase B: IPA-HPLC; Flow rate: 20 mL/min; Gradient: isocratic 40% Mobile Phase B; Wave Length: 220/254 nm; RT1 (min): 10.614; RT2 (min): 20.977; Sample Solvent: EtOH-HPLC; Injection Volume: 3 mL; Number Of Runs: 1). The second eluting isomer was concentrated and lyophilized to afford Example 40 (4.7 mg, 26% yield) as a white solid. The first eluting isomer was concentrated and lyophilized to afford Example 41 (2.8 mg 15% yield) as a white solid.

Example 40: ¹H NMR (300 MHz, DMSO-d₆) δ: 10.89 (s, 1H), 8.69 (d, J=2.7 Hz, 1H), 8.63-8.65 (m, 2H), 7.75 (t, J=71.7 Hz, 1H), 7.64 (s, 1H), 7.41 (d, J=0.6 Hz, 1H), 6.57 (d, J=4.8 Hz, 1H), 4.49-4.52 (m, 1H), 3.75 (s, 3H), 2.84-2.86 (m, 1H), 2.47-2.53 (m, 1H), 1.96 (s, 3H). LC-MS: m/z 483 [M+H]⁺.

Example 41: ¹H NMR (300 MHz, DMSO-d₆) δ:10.89 (s, 1H), 8.69 (d, J=2.7 Hz, 1H), 8.63-8.64 (m, 2H), 7.75 (t, J=71.7 Hz, 1H), 7.64 (s, 1H), 7.41 (d, J=0.6 Hz, 1H), 6.58 (d, J=5.1 Hz, 1H), 4.46-4.52 (m, 1H), 3.75 (s, 3H), 2.84-2.91 (m, 1H), 2.47-2.53 (m, 1H), 1.96 (s, 3H). LC-MS: m/z 483 [M+H]⁺.

### Step 3: Separation of Enantiomers to Obtain Example 42 and Example 43

12.4 mg of cis-N-(5-cyano-6-(difluoromethoxy)pyridin-3-yl)-2-fluoro-8-methyl-8-(1-methyl-1H-pyrazol-4-yl)-7,8-dihydro-6H-cyclopenta[e]pyrazolo[1,5-a]pyrimidine-6-carboxamide were submitted to chiral HPLC purification (Column: CHIRALPAK IF, 2*25 cm, 5 μm; Mobile Phase A: Hex (0.1% FA)-HPLC, Mobile Phase B: EtOH-HPLC; Flow rate: 20 mL/min; Gradient: isocratic 30% Mobile Phase B; Wave Length: 220/254 nm; RT1 (min): 9.371; RT2 (min): 18.431; Sample Solvent: EtOH-HPLC; Injection Volume: 2.5 mL; Number Of Runs: 1). The second eluting isomer was concentrated and lyophilized to afford Example 42 (5.4 mg, 28 yield) as a white solid. The first eluting isomer was concentrated and lyophilized to afford Example 43 (2.6 mg, 13 yield) as a white solid.

Example 42: ¹H NMR (300 MHz, DMSO-d₆) δ:10.86 (s, 1H), 8.68 (d, J=2.7 Hz, 1H), 8.67 (s, 1H), 8.61 (d, J=2.7 Hz, 1H), 7.75 (t, J=71.4 Hz, 1H), 7.62 (s, 1H), 7.33 (d, J=0.6 Hz, 1H), 6.54 (d, J=5.1 Hz, 1H), 4.46-4.51 (m, 1H), 3.75 (s, 3H), 2.66-2.80 (m, 2H), 1.86 (s, 3H). LC-MS: m/z 483 [M+H]⁺.

Example 43: ¹H NMR (300 MHz, DMSO-d₆) δ:10.89 (s, 1H), 8.68 (d, J=2.4 Hz, 1H), 8.67 (s, 1H), 8.61 (d, J=2.7 Hz, 1H), 7.75 (t, J=71.4 Hz, 1H), 7.62 (s, 1H), 7.33 (d, J=0.9 Hz, 1H), 6.54 (d, J=5.1 Hz, 1H), 4.46-4.49 (m, 1H), 3.75 (s, 3H), 2.69-2.79 (m, 2H), 1.86 (s, 3H). LC-MS: m/z 483 [M+H]⁺.

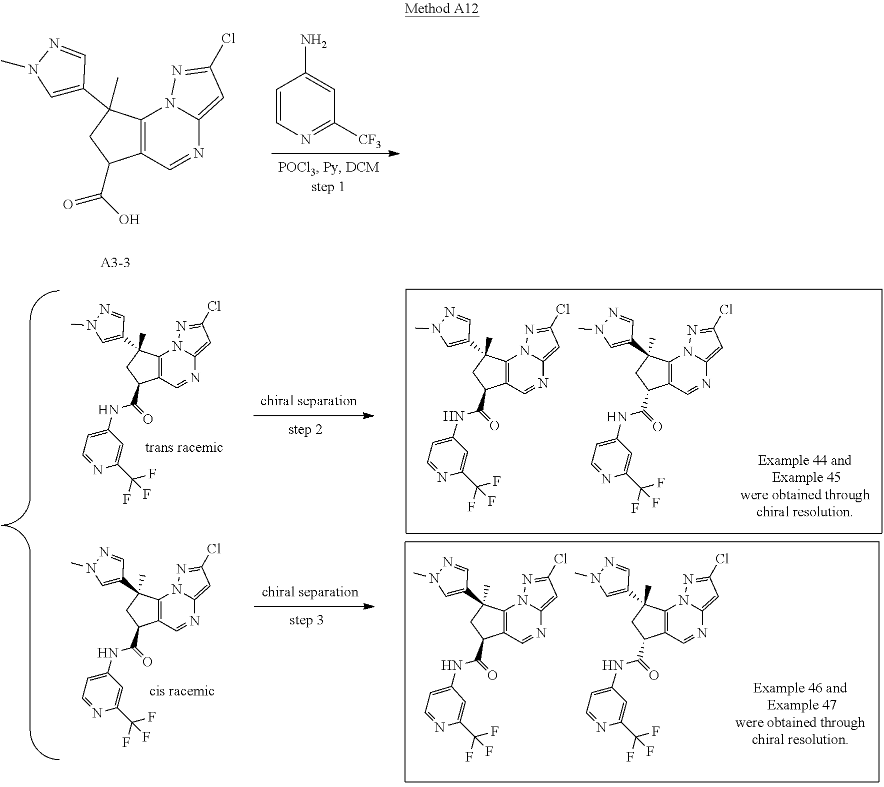

### Examples 44, 45: (6S,8R)-2-chloro-8-methyl-8-(1-methyl-1H-pyrazol-4-yl)-N-(2-(trifluoromethyl)pyridin-4-yl)-7,8-dihydro-6H-cyclopenta[e]pyrazolo[1,5-a]pyrimidine-6-carboxamide; (6R,8S)-2-chloro-8-methyl-8-(1-methyl-1H-pyrazol-4-yl)-N-(2-(trifluoromethyl)pyridin-4-yl)-7,8-dihydro-6H-cyclopenta[e]pyrazolo[1,5-a]pyrimidine-6-carboxamide

### Examples 46, 47: (6S,8S)-2-chloro-8-methyl-8-(1-methyl-1H-pyrazol-4-yl)-N-(2-(trifluoromethyl)pyridin-4-yl)-7,8-dihydro-6H-cyclopenta[e]pyrazolo[1,5-a]pyrimidine-6-carboxamide; (6R,8R)-2-chloro-8-methyl-8-(1-methyl-1H-pyrazol-4-yl)-N-(2-(trifluoromethyl)pyridin-4-yl)-7,8-dihydro-6H-cyclopenta[e]pyrazolo[1,5-a]pyrimidine-6-carboxamide

The relative and absolute stereochemistry of each example was not determined.

### Step 1: Trans-2-chloro-8-methyl-8-(1-methyl-1H-pyrazol-4-yl)-N-(2-(trifluoromethyl)pyridin-4-yl)-7,8-dihydro-6H-cyclopenta[e]pyrazolo[1,5-a]pyrimidine-6-carboxamide and cis-2-chloro-8-methyl-8-(1-methyl-1H-pyrazol-4-yl)-N-(2-(trifluoromethyl)pyridin-4-yl)-7,8-dihydro-6H-cyclopenta[e]pyrazolo[1,5-a]pyrimidine-6-carboxamide

To a stirred solution of 2-chloro-8-methyl-8-(1-methyl-1H-pyrazol-4-yl)-7,8-dihydro-6H-cyclopenta[e]pyrazolo[1,5-a]pyrimidine-6-carboxylic acid (A3-3, 90 mg, 211 mol) in DCM (15 mL) were added pyridine (139 mg, 1.7 mmol) and phosphoryl trichloride (178 mg, 1.2 mmol) under nitrogen atmosphere. The reaction mixture was stirred at 25° C. for 1 h. 2-(trifluoromethyl)pyridin-4-amine (69 mg, 423 mol) was added, and the mixture was stirred at 0° C. for 3 h. The reaction mixture was concentrated under reduced pressure, and the residue was diluted with water (10 mL). The resulting mixture was extracted with ethyl acetate (3×10 mL). The combined organic layers were dried over anhydrous sodium sulfate and concentrated under reduced pressure. The residue was applied on a silica gel column and eluted with DCM/MeOH (10:1) to give the crude product, which was further purified by prep-HPLC to afford racemic mixtures of the title compounds: trans isomer (19 mg 14% yield) as a white solid, LC-MS: m/z 476 [M+H]⁺; cis-isomer (11 mg, 8% yield) as a white solid, LC-MS: m/z 476 [M+H]⁺.

### Step 2: Separation of Enantiomers to Obtain Example 44 and Example 45

10 mg of trans-2-chloro-8-methyl-8-(1-methyl-1H-pyrazol-4-yl)-N-(2-(trifluoromethyl)pyridin-4-yl)-7,8-dihydro-6H-cyclopenta[e]pyrazolo[1,5-a]pyrimidine-6-carboxamide were submitted to chiral HPLC purification (Column: CHIRAL ART Cellulose-SC, 2*25 cm, 5 μm; Mobile Phase A: Hex (0.1% FA)-HPLC, Mobile Phase B: IPA-HPLC; Flow rate: 20 mL/min; Gradient: isocratic 30% Mobile Phase B; Wave Length: 220/254 nm; RT1 (min): 9.391; RT2 (min): 12.872; Sample Solvent: EtOH-HPLC; Injection Volume: 1 mL; Number Of Runs: 5). The second eluting isomer was concentrated and lyophilized to afford Example 44 (3.7 mg, 36% yield) as a white solid. The first eluting isomer was concentrated and lyophilized to afford Example 45 (3.1 mg, 30% yield) as a white solid.

Example 44: ¹H NMR (300 MHz, DMSO-d₆) 5:11.12 (s, 1H), 8.67 (d, J=6.3 Hz, 1H), 8.65 (s, 1H), 8.19 (d, J=1.8 Hz, 1H), 7.80-7.83 (m, 1H), 7.61 (s, 1H), 7.40 (s, 1H), 6.96 (s, 1H), 4.49-4.55 (m, 1H), 3.76 (s, 3H), 2.85-2.92 (m, 1H), 2.47-2.52 (m, 1H), 1.98 (s, 3H). LC-MS: m/z 476 [M+H]⁺.

Example 45: ¹H NMR (300 MHz, DMSO-d₆) δ: 11.11 (s, 1H), 8.67 (d, J=6.0 Hz, 1H), 8.65 (s, 1H), 8.19 (d, J=2.0 Hz, 1H), 7.80-7.83 (m, 1H), 7.61 (s, 1H), 7.40 (s, 1H), 6.96 (s, 1H), 4.49-4.52 (m, 1H), 3.76 (s, 3H), 2.85-2.92 (m, 1H), 2.48-2.54 (m, 1H), 1.98 (s, 3H). LC-MS: m/z 476 [M+H]⁺.

### Step 10: Separation of Enantiomers to Obtain Example 46 and Example 47

10 mg of cis-2-chloro-8-methyl-8-(1-methyl-1H-pyrazol-4-yl)-N-(2-(trifluoromethyl)pyridin-4-yl)-7,8-dihydro-6H-cyclopenta[e]pyrazolo[1,5-a]pyrimidine-6-carboxamide were submitted to chiral HPLC purification (Column: CHIRALPAK IG, 2*25 cm, 5 μm; Mobile Phase A: Hex (0.1% FA)-HPLC, Mobile Phase B: IPA-HPLC; Flow rate: 20 mL/min; Gradient: isocratic 20% Mobile Phase B; Wave Length: 220/254 nm; RT1 (min): 6.723; RT2 (min): 10.474; Sample Solvent: EtOH-HPLC; Injection Volume: 1 mL; Number Of Runs: 5). The first eluting isomer was concentrated and lyophilized to afford Example 46 (3.7 mg, 36% yield) as a white solid. The second eluting isomer was concentrated and lyophilized to afford Example 47 (4.5 mg, 44% yield) as a white solid.

Example 46: ¹H NMR (300 MHz, DMSO-d₆) δ: 11.12 (s, 1H), 8.70 (s, 1H), 8.65 (d, J=5.4 Hz, 1H), 8.15 (d, J=1.5 Hz, 1H), 7.82-7.84 (m, 111), 7.59 (s, 1H),7.31 (d, J=0.9 Hz, 111), 6.93 (s, 1H), 4.49-4.56 (m, 1H), 3.74 (s, 3H), 2.80-2.87 (m, 1H), 2.62-2.69 (m, 1H), 1.89 (s, 3H). LC-MS: m/z 476 [M+H]⁺.

Example 47: ¹H NMR (300 MHz, DMSO-d₆) δ: 11.14 (s, 1H), 8.70 (s, 1H), 8.65 (d, J=5.4 Hz, 1H), 8.15 (d, J=1.5 Hz, 1H), 7.81-7.84 (m, 1H), 7.59 (s, 1H),7.31 (d, J=0.9 Hz, 1H), 6.93 (s, 1H), 4.51-4.53 (m, 1H), 3.74 (s, 3H), 2.80-2.84 (m, 1H), 2.62-2.69 (m, 1H), 1.89 (s, 3H). LC-MS: m/z 476 [M+H]⁺.

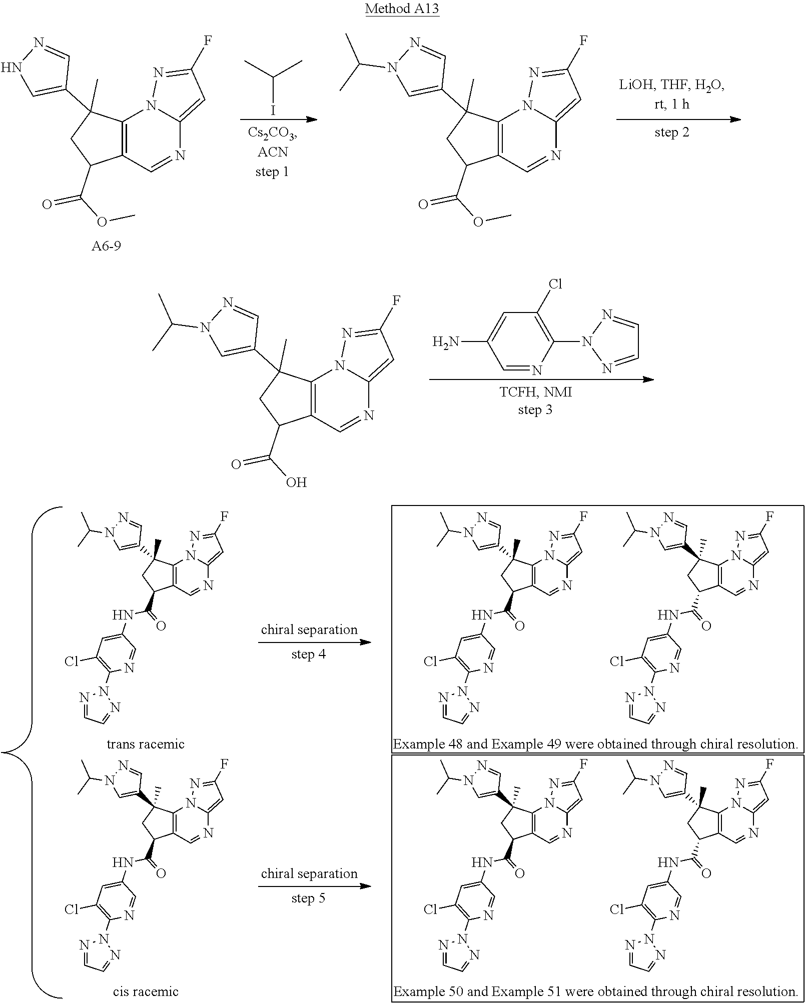

### Examples 48, 49: (6S,8R)—N-(5-chloro-6-(2H-1,2,3-triazol-2-yl)pyridin-3-yl)-2-fluoro-8-(1-isopropyl-1H-pyrazol-4-yl)-8-methyl-7,8-dihydro-6H-cyclopenta[e]pyrazolo[1,5-a]pyrimidine-6-carboxamide; (6R,8S)—N-(5-chloro-6-(2H-1,2,3-triazol-2-yl)pyridin-3-yl)-2-fluoro-8-(1-isopropyl-1H-pyrazol-4-yl)-8-methyl-7,8-dihydro-6H-cyclopenta[e]pyrazolo[1,5-a]pyrimidine-6-carboxamide

### Examples 50, 51: (6S,8S)—N-(5-chloro-6-(2H-1,2,3-triazol-2-yl)pyridin-3-yl)-2-fluoro-8-(1-isopropyl-1H-pyrazol-4-yl)-8-methyl-7,8-dihydro-6H-cyclopenta[e]pyrazolo[1,5-a]pyrimidine-6-carboxamide; (6R,8R)—N-(5-chloro-6-(2H-1,2,3-triazol-2-yl)pyridin-3-yl)-2-fluoro-8-(1-isopropyl-1H-pyrazol-4-yl)-8-methyl-7,8-dihydro-6H-cyclopenta[e]pyrazolo[1,5-a]pyrimidine-6-carboxamide

The relative and absolute stereochemistry of each example was not determined.

### Step 1: Methyl 2-fluoro-8-(1-isopropyl-1H-pyrazol-4-yl)-8-methyl-7,8-dihydro-6H-cyclopenta[e]pyrazolo[1,5-a]pyrimidine-6-carboxylate

To a stirred solution of methyl 2-fluoro-8-methyl-8-(1H-pyrazol-4-yl)-7,8-dihydro-6H-cyclopenta[e]pyrazolo[1,5-a]pyrimidine-6-carboxylate (A6-9, 180 mg, 571 mol) in ACN (5 mL) was added cesium carbonate (372 mg, 1.1 mmol) at 0° C. The mixture was stirred at 0° C. for 20 min. 2-iodopropane (1.9 g, 11.4 mmol) in ACN (0.5 mL) was added, and the mixture was stirred at 25° C. for 5 h. The reaction was quenched with water (20 mL). The resulting mixture was extracted with ethyl acetate (3×20 mL). The combined organic layers were dried over anhydrous sodium sulfate and concentrated under reduced pressure. The residue was applied on a silica gel column and eluted with DCM/MeOH (10:1) to give the title compound (80 mg, 27% yield) as a yellow oil. LC-MS (ES, m/z): 358[M+H]⁺.

### Step 2: 2-fluoro-8-(1-isopropyl-1H-pyrazol-4-yl)-8-methyl-7,8-dihydro-6H-cyclopenta[e]pyrazolo[1,5-a]pyrimidine-6-carboxylic acid

In analogy to Method A6 step 11 but using methyl 2-fluoro-8-(1-isopropyl-1H-pyrazol-4-yl)-8-methyl-7,8-dihydro-6H-cyclopenta[e]pyrazolo[1,5-a]pyrimidine-6-carboxylate (75 mg, 210 mol), the crude title compound was obtained as a yellow oil (80 mg, 70% yield). LC-MS (ES, m/z): 344[M+H]⁺.

### Step 3: trans-N-(5-chloro-6-(2H-1,2,3-triazol-2-yl)pyridin-3-yl)-2-fluoro-8-(1-isopropyl-1H-pyrazol-4-yl)-8-methyl-7,8-dihydro-6H-cyclopenta[e]pyrazolo[1,5-a]pyrimidine-6-carboxamide and cis-N-(5-chloro-6-(2H-1,2,3-triazol-2-yl)pyridin-3-yl)-2-fluoro-8-(1-isopropyl-1H-pyrazol-4-yl)-8-methyl-7,8-dihydro-6H-cyclopenta[e]pyrazolo[1,5-a]pyrimidine-6-carboxamide

In analogy to Method A1 step 8 but using 2-fluoro-8-(1-isopropyl-1H-pyrazol-4-yl)-8-methyl-7,8-dihydro-6H-cyclopenta[e]pyrazolo[1,5-a]pyrimidine-6-carboxylic acid (75 mg, 218 μmol) and 5-chloro-6-(triazol-2-yl)pyridin-3-amine (64 mg, 328 μmol), racemic mixtures of the title compounds were obtained: trans isomer (13.9 mg, 11% yield) as a white solid, LC-MS (ES, m/z): 521[M+H]⁺; cis-isomer (6.7 mg, 5% yield) as a white solid, LC-MS (ES, m/z): 521[M+H]⁺.

### Step 4: Separation of Enantiomers to Obtain Example 48 and Example 49

12.8 mg of trans-N-(5-chloro-6-(2H-1,2,3-triazol-2-yl)pyridin-3-yl)-2-fluoro-8-(1-isopropyl-1H-pyrazol-4-yl)-8-methyl-7,8-dihydro-6H-cyclopenta[e]pyrazolo[1,5-a]pyrimidine-6-carboxamide were submitted to chiral HPLC purification (Column: CHIRALPAK IF, 2*25 cm, 5 μm; Mobile Phase A: Hex (0.1% FA)-HPLC, Mobile Phase B: EtOH-HPLC; Flow rate: 20 mL/min; Gradient: isocratic 50% Mobile Phase B; Wave Length: 220/254 nm; RT1 (min): 11.401; RT2 (min): 17.85; Sample Solvent: EtOH-HPLC; Injection Volume: 1 mL; Number Of Runs: 2).

The second eluting isomer was concentrated and lyophilized to afford Example 48 (2.6 mg, 20% yield) as a white solid. The first eluting isomer was concentrated and lyophilized to afford Example 49 (2.0 mg, 15% yield) as a white solid.

Example 48: ¹H NMR (400 MHz, Chloroform-d) b 8.55-8.77 (m, 4H), 7.94-8.00 (m, 3H), 7.83 (s, 1H), 6.32 (d, J=5.2 Hz, 1H), 4.48-4.68 (m, 2H), 3.20-3.33 (m, 1H), 2.60-2.72 (m, 1H), 2.12 (s, 3H), 1.56-1.59 (m, 6H). LC-MS (ES, m/z): 521[M+H]⁺.

Example 49: ¹H NMR (400 MHz, Chloroform-d) b 8.64-8.86 (m, 2H), 8.52-8.67 (m, 2H), 7.96 (s, 2H), 7.67-7.90 (m, 2H), 6.31 (d, J=5.2 Hz, 1H), 4.43-4.64 (m, 2H), 3.15-3.28 (m, 1H), 2.59-2.74 (m, 1H), 2.12 (s, 3H), 1.51-1.61 (m, 6H). LC-MS (ES, m/z): 521 [M+H]⁺.

### Step 5: Separation of Enantiomers to Obtain Example 50 and Example 51

5.6 mg of cis-N-(5-chloro-6-(2H-1,2,3-triazol-2-yl)pyridin-3-yl)-2-fluoro-8-(1-isopropyl-1H-pyrazol-4-yl)-8-methyl-7,8-dihydro-6H-cyclopenta[e]pyrazolo[1,5-a]pyrimidine-6-carboxamide were submitted to chiral HPLC purification (Column: CHIRALPAK IF, 2*25 cm, 5 μm; Mobile Phase A: Hex (0.1% FA)-HPLC, Mobile Phase B: EtOH-HPLC; Flow rate: 20 mL/min; Gradient: isocratic 50% Mobile Phase B; Wave Length: 220/254 nm; RT1 (min): 7.898; RT2 (min): 14.194; Sample Solvent: EtOH-HPLC; Injection Volume: 1 mL; Number Of Runs: 1). The second eluting isomer was concentrated and lyophilized to afford Example 50 (1.0 mg, 17% yield) as a white solid. The first eluting isomer was concentrated and lyophilized to afford Example 51 (1.9 mg, 31% yield) as a white solid.

Example 50: ¹H NMR (400 MHz, Chloroform-d) b 8.46-8.71 (m, 3H), 7.72-7.99 (m, 4H), 6.32 (d, J=5.2 Hz, 1H), 4.51-4.74 (m, 2H), 2.83-3.10 (m, 2H), 2.01 (s, 3H), 1.53-1.56 (m, 6H). LC-MS (ES, m/z): 521[M+H]⁺.

Example 51: ¹H NMR (400 MHz, Chloroform-d) b 8.29-9.05 (m, 4H), 7.64-7.99 (m, 4H), 6.31 (d, J=5.2 Hz, 1H), 4.49-4.70 (m, 2H), 2.85-3.08 (m, 2H), 2.01 (s, 3H), 1.51-1.57 (m, 6H). LC-MS (ES, m/z): 521[M+H]⁺.

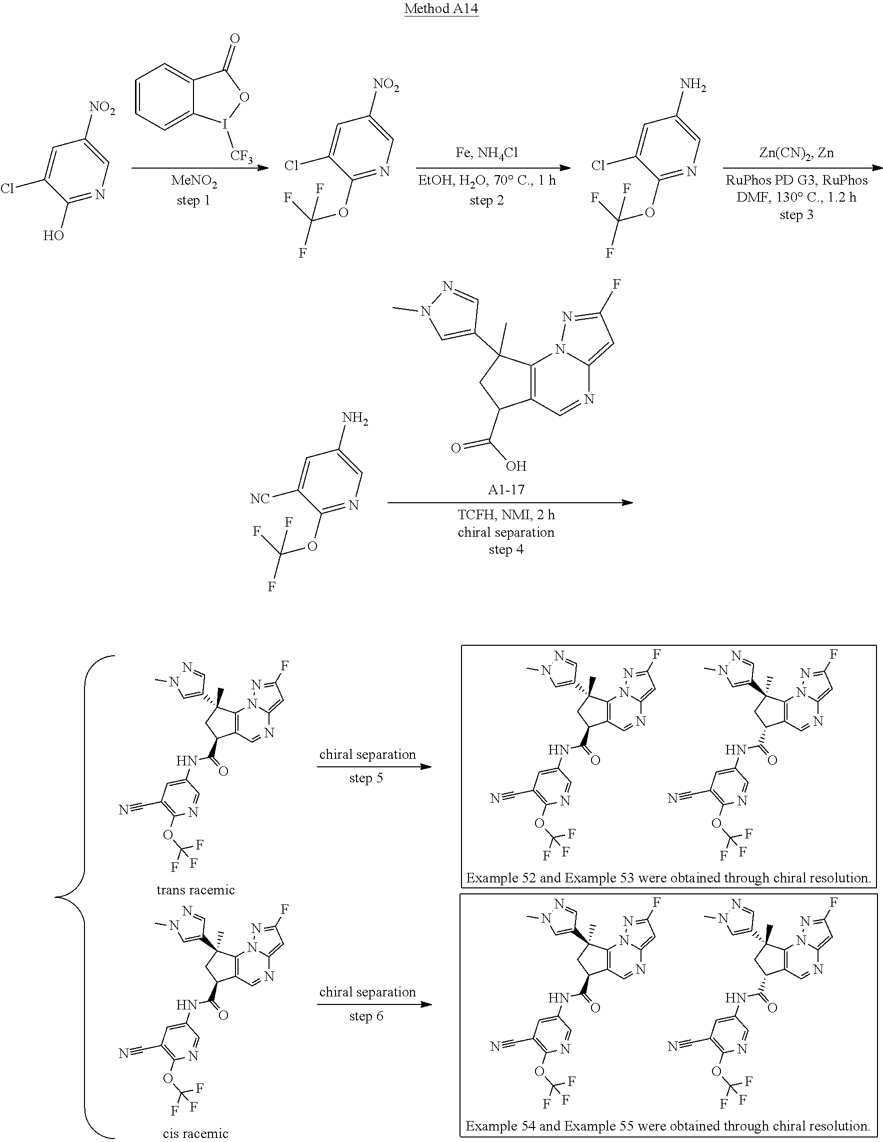

### Examples 52, 53: (6S,8R)—N-(5-cyano-6-(trifluoromethoxy)pyridin-3-yl)-2-fluoro-8-methyl-8-(1-methyl-1H-pyrazol-4-yl)-7,8-dihydro-6H-cyclopenta[e]pyrazolo[1,5-a]pyrimidine-6-carboxamide; (6R,8S)—N-(5-cyano-6-(trifluoromethoxy)pyridin-3-yl)-2-fluoro-8-methyl-8-(1-methyl-1H-pyrazol-4-yl)-7,8-dihydro-6H-cyclopenta[e]pyrazolo[1,5-a]pyrimidine-6-carboxamide

### Examples 54, 55: (6S,8S)—N-(5-cyano-6-(trifluoromethoxy)pyridin-3-yl)-2-fluoro-8-methyl-8-(1-methyl-1H-pyrazol-4-yl)-7,8-dihydro-6H-cyclopenta[e]pyrazolo[1,5-a]pyrimidine-6-carboxamide; (6R,8R)—N-(5-cyano-6-(trifluoromethoxy)pyridin-3-yl)-2-fluoro-8-methyl-8-(1-methyl-1H-pyrazol-4-yl)-7,8-dihydro-6H-cyclopenta[e]pyrazolo[1,5-a]pyrimidine-6-carboxamide

The relative and absolute stereochemistry of each example was not determined.

### Step 1: 3-chloro-5-nitro-2-(trifluoromethoxy)pyridine

To a mixture of 3-chloro-5-nitropyridin-2-ol (3 g, 17.2 mmol) in MeNO₂ (30 mL) was added 1-(trifluoromethyl)-113-benzo[d][1,2]iodaoxol-3(1H)-one (10.9 g, 34.4 mmol) in portions at 100° C. The mixture was stirred at 100° C. for 1 h. The mixture was cooled to room temperature and poured into water (200 mL) after. The mixture was extracted with EtOAc (3×200 mL). The combined organic layers were dried over anhydrous sodium sulfate and concentrated under reduced pressure. The residue was applied onto a silica gel column and eluted with EtOAc/PE (1:15) to give the title compound (1.3 g, 26% yield) as a yellow oil. LC-MS: m/z 243 [M+H]⁺.

### Step 2: 5-chloro-6-(trifluoromethoxy)pyridin-3-amine

To a stirred solution of 3-chloro-5-nitro-2-(trifluoromethoxy)pyridine (900 mg, 3.7 mmol) in EtOH/H₂O (3:1, 20 mL) were added iron (1.04 g, 18.5 mmol) and NH₄Cl (6.35 g, 118.7 mmol). The resulting mixture was stirred at 70° C. for 1 h. The mixture was filtered, the filtrate diluted with water (100 mL), and the resulting mixture was extracted with ethyl acetate (3×100 mL). The combined organic layers were dried over anhydrous sodium sulfate and concentrated under reduced pressure to afford the crude title compound (330 mg, 41% yield) as a yellow solid. LC-MS: m/z 213 [M+H]⁺.

### Step 3: 5-amino-2-(trifluoromethoxy)nicotinonitrile

To a stirred solution of 5-chloro-6-(trifluoromethoxy)pyridin-3-amine (200 mg, 0.9 mmol) in DMF (8 mL) were added Zn(CN)₂ (221 mg, 1.9 mmol), Zn (6.1 mg, 94 mol), RuPhos-Pd G3 (43.9 mg, 94 μmol) and RuPhos (76.8 mg, 94 μmol). The resulting mixture was stirred at 130° C. for 1.2 h under nitrogen. The mixture was cooled to 25° C., and water (100 mL) was added. The resulting mixture was extracted with ethyl acetate (3×100 mL). The combined organic layers were dried over anhydrous sodium sulfate and concentrated under reduced pressure. The residue was applied onto a silica gel column and eluted with EtOAc/PE (1:3) to give the title compound (110 mg, 57% yield) as a yellow solid. LC-MS: m/z 204 [M+H]⁺.

### Step 4: trans-N-(5-cyano-6-(trifluoromethoxy)pyridin-3-yl)-2-fluoro-8-methyl-8-(1-methyl-1H-pyrazol-4-yl)-7,8-dihydro-6H-cyclopenta[e]pyrazolo[1,5-a]pyrimidine-6-carboxamide and cis-N-(5-cyano-6-(trifluoromethoxy)pyridin-3-yl)-2-fluoro-8-methyl-8-(1-methyl-1H-pyrazol-4-yl)-7,8-dihydro-6H-cyclopenta[e]pyrazolo[1,5-a]pyrimidine-6-carboxamide

In analogy to Method A1 step 8 but using 2-fluoro-8-methyl-8-(1-methyl-1H-pyrazol-4-yl)-7,8-dihydro-6H-cyclopenta[e]pyrazolo[1,5-a]pyrimidine-6-carboxylic acid (A1-7, 72.4 mg, 229 μmol) and 5-amino-2-(trifluoromethoxy)nicotinonitrile (56 mg, 275 μmol), racemic mixtures of the title compounds were obtained: trans isomer (25 mg, 21% yield) as an off-white solid, LC-MS: m/z 501 [M+H]⁺; cis-isomer (25 mg, 21% yield) as a white solid, LC-MS: m/z 501 [M+H]⁺.

### Step 5: Separation of Enantiomers to Obtain Example 52 and Example 53

20 mg of trans-N-(5-cyano-6-(trifluoromethoxy)pyridin-3-yl)-2-fluoro-8-methyl-8-(1-methyl-1H-pyrazol-4-yl)-7,8-dihydro-6H-cyclopenta[e]pyrazolo[1,5-a]pyrimidine-6-carboxamide were submitted to chiral HPLC purification (Column: CHIRALPAK IF, 2*25 cm, 5 m; Mobile Phase A: Hex (0.1% FA)-HPLC, Mobile Phase B: IPA-HPLC; Flow rate: 20 mL/min; Gradient: isocratic 40% Mobile Phase B; Wave Length: 220/254 nm; RT1 (min): 6.674; RT2 (min): 18.161; Sample Solvent: EtOH-HPLC; Injection Volume: 2 mL; Number Of Runs: 2). The second eluting isomer was concentrated and lyophilized to afford Example 52 (12.6 mg, 62% yield) as a white solid. The first eluting isomer was concentrated and lyophilized to afford Example 53 (5.7 mg, 28% yield) as a white solid.

Example 52: ¹HNMR (400 MHz, DMSO-d₆) δ: 11.06 (s, 1H), 8.74-8.76 (m, 2H), 8.62 s, 1H), 7.64 (s, 1H), 7.41 (s, 1H), 6.56 (d, J=4.0 Hz, 1H), 4.48-4.52 (m, 1H), 3.75 (s, 3H), 2.84-2.89 (m, 1H), 2.48-2.49 (m, 1H), 1.95 (s, 3H). LC-MS: m/z 501 [M+H]⁺.

Example 53: ¹HNMR (400 MHz, DMSO-d₆) δ: 11.07 (s, 1H), 8.75-8.76 (m, 2H), 8.62 s, 1H), 7.64 (s, 1H), 7.41 (s, 1H), 6.56 (d, J=4.0 Hz, 1H), 4.48-4.53 (m, 1H), 3.75 (s, 3H), 2.84-2.89 (m, 1H), 2.48-2.49 (m, 1H), 1.95 (s, 311). LC-MS: m/z 501 [M+H]⁺.

### Step 6: Separation of Enantiomers to Obtain Example 54 and Example 55

20 mg of cis-N-(5-cyano-6-(trifluoromethoxy)pyridin-3-yl)-2-fluoro-8-methyl-8-(1-methyl-1H-pyrazol-4-yl)-7,8-dihydro-6H-cyclopenta[e]pyrazolo[1,5-a]pyrimidine-6-carboxamide were submitted to chiral HPLC purification (Column: CHIRALPAK IF, 2*25 cm, 5 m; Mobile Phase A: Hex (0.1% FA)-HPLC, Mobile Phase B: EtOH-HPLC; Flow rate: 20 mL/min; Gradient: isocratic 50% Mobile Phase B; Wave Length: 220/254 nm; RT1 (min): 4.92; RT2 (min): 11.085; Sample Solvent: EtOH-HPLC; Injection Volume: 1 mL; Number Of Runs: 3). The second eluting isomer was concentrated and lyophilized to afford Example 54 (2.3 mg, 11% yield) as a white solid. The first eluting isomer was concentrated and lyophilized to afford Example 55 (2.9 mg, 14% yield) as a white solid.

Example 54: ¹HNMR (400 MHz, DMSO-d₆) δ: 11.08 (s, 1H), 8.72-8.76 (m, 2H), 8.67 s, 1H), 7.61 (s, 1H), 7.32 (s, 1H), 6.54 (d, J=4.8 Hz, 1H), 4.48-4.52 (m, 1H), 3.75 (s, 3H), 2.77-2.83 (m, 1H), 2.64-2.69 (m, 1H), 1.85 (s, 3H). LC-MS: m/z 501 [M+H]⁺.

Example 55: ¹HNMR (400 MHz, DMSO-d₆) δ: 11.03 (s, 1H), 8.71-8.75 (m, 2H), 8.67 s, 1H), 7.61 (s, 1H), 7.32 (s, 1H), 6.54 (d, J=4.8 Hz, 1H), 4.48-4.51 (m, 1H), 3.75 (s, 3H), 2.78-2.84 (m, 1H), 2.65-2.70 (m, 1H), 1.85 (s, 3H). LC-MS: m/z 501 [M+H]⁺.

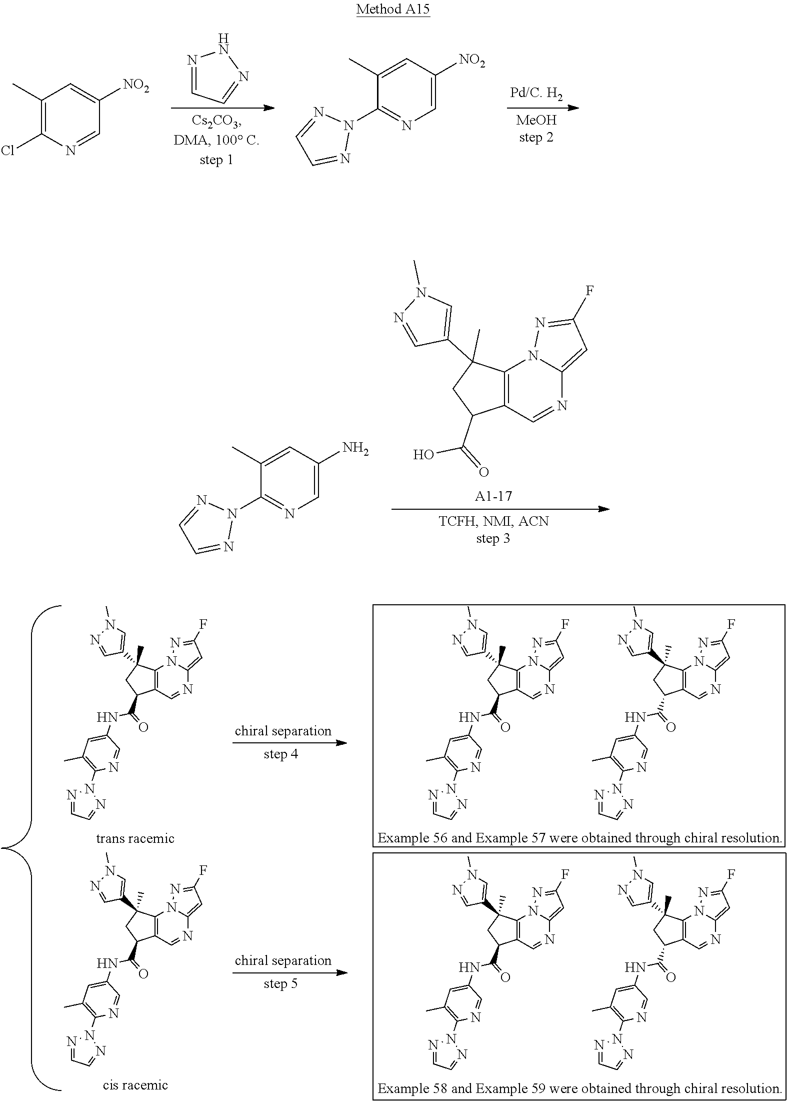

### Examples 56, 57: (6S,8R)-2-fluoro-8-methyl-8-(1-methyl-1H-pyrazol-4-yl)-N-(5-methyl-6-(2H-1,2,3-triazol-2-yl)pyridin-3-yl)-7,8-dihydro-6H-cyclopenta[e]pyrazolo[1,5-a]pyrimidine-6-carboxamide; (6R,8S)-2-fluoro-8-methyl-8-(1-methyl-1H-pyrazol-4-yl)-N-(5-methyl-6-(2H-1,2,3-triazol-2-yl)pyridin-3-yl)-7,8-dihydro-6H-cyclopenta[e]pyrazolo[1,5-a]pyrimidine-6-carboxamide

### Examples 58, 59: (6S,8S)-2-fluoro-8-methyl-8-(1-methyl-1H-pyrazol-4-yl)-N-(5-methyl-6-(2H-1,2,3-triazol-2-yl)pyridin-3-yl)-7,8-dihydro-6H-cyclopenta[e]pyrazolo[1,5-a]pyrimidine-6-carboxamide; (6R,8R)-2-fluoro-8-methyl-8-(1-methyl-1H-pyrazol-4-yl)-N-(5-methyl-6-(2H-1,2,3-triazol-2-yl)pyridin-3-yl)-7,8-dihydro-6H-cyclopenta[e]pyrazolo[1,5-a]pyrimidine-6-carboxamide

The relative and absolute stereochemistry of each example was not determined.

### Step 1: 3-methyl-5-nitro-2-(2H-1,2,3-triazol-2-yl)pyridine

To a stirred mixture of 2-chloro-3-methyl-5-nitropyridine (10.0 g, 57.9 mmol) and 2H-1,2,3-triazole (4.8 g, 69.5 mmol) in acetonitrile (100 mL) was added K₂CO₃ (16.0 g, 115.9 mmol). The reaction mixture was stirred at 25° C. for 24 h. The resulting mixture was concentrated under reduced pressure. Water (100 mL) was added, and the resulting mixture was extracted with ethyl acetate (3×100 mL). The combined organic layers were washed with water (3×100 mL), dried over anhydrous sodium sulfate and concentrated under reduced pressure. The residue was applied onto a silica gel column and eluted with EtOAc/PE (5:1) to afford the title compound (10.0 g, 84% yield) as a yellow solid. LC-MS: m/z 206 [M+H]⁺.

### Step 2: 5-methyl-6-(2H-1,2,3-triazol-2-yl)pyridin-3-amine

To a stirred solution of 3-methyl-5-nitro-2-(2H-1,2,3-triazol-2-yl)pyridine (10.0 g, 48.7 mmol) in methanol (50 mL) was added Pd/C (11.8 g, 97.4 mmol). The reaction mixture was degassed and then stirred at 25° C. for 72 h under hydrogen. The reaction mixture was filtered, and the filtrate was concentrated under reduced pressure. The residue was applied onto a silica gel column and eluted with EtOAc/PE (1:1) to afford the title compound (4.0 g, 46% yield) as a yellow oil. ¹H NMR (400 MHz, DMSO-d₆) δ 8.00 (s, 2H), 7.71 (d, J=2.8 Hz, 1H), 6.95 (d, J=2.8, 1H), 5.75 (s, 2H), 1.96 (s, 3H). LC-MS: m/z 176 [M+H]⁺.

### Step 3: trans-2-fluoro-8-methyl-8-(1-methyl-1H-pyrazol-4-yl)-N-(5-methyl-6-(2H-1,2,3-triazol-2-yl)pyridin-3-yl)-7,8-dihydro-6H-cyclopenta[e]pyrazolo[1,5-a]pyrimidine-6-carboxamide and cis-2-fluoro-8-methyl-8-(1-methyl-1H-pyrazol-4-yl)-N-(5-methyl-6-(2H-1,2,3-triazol-2-yl)pyridin-3-yl)-7,8-dihydro-6H-cyclopenta[e]pyrazolo[1,5-a]pyrimidine-6-carboxamide

In analogy to Method A1 step 8 but using 2-fluoro-8-methyl-8-(1-methyl-1H-pyrazol-4-yl)-7,8-dihydro-6H-cyclopenta[e]pyrazolo[1,5-a]pyrimidine-6-carboxylic acid (A1-7, 80 mg, 254 μmol) and 5-methyl-6-(triazol-2-yl)pyridin-3-amine (44 mg, 254 μmol), racemic mixtures of the title compounds were obtained: trans isomer (26 mg, 16% yield) as a white solid, LC-MS: m/z 473 [M+H]⁺; cis-isomer (22 mg, 15% yield) as a white solid, LC-MS: m/z 473 [M+H]⁺.

### Step 4: Separation of Enantiomers to Obtain Example 56 and Example 57

26 mg of trans-2-fluoro-8-methyl-8-(1-methyl-1H-pyrazol-4-yl)-N-(5-methyl-6-(2H-1,2,3-triazol-2-yl)pyridin-3-yl)-7,8-dihydro-6H-cyclopenta[e]pyrazolo[1,5-a]pyrimidine-6-carboxamide were submitted to chiral HPLC purification (Column: CHIRALPAK IF, 2*25 cm, 5 m; Mobile Phase A: MtBE (0.5% 2M NH3-MeOH)-HPLC, Mobile Phase B: EtOH-HPLC; Flow rate: 17 mL/min; Gradient: isocratic 50% Mobile Phase B; Wave Length: 220/254 nm; RT1 (min): 9.155; RT2 (min): 17.671; Sample Solvent: EtOH-HPLC; Injection Volume: 2 mL; Number Of Runs: 1). The second eluting isomer was concentrated and lyophilized to afford Example 56 (6.4 mg, 24% yield) as a white solid. The first eluting isomer was concentrated and lyophilized to afford Example 57 (6.7 mg, 25.5% yield) as a white solid.

Example 56: ¹H NMR (400 MHz, DMSO-d₆) δ10.82 (s, 1H), 8.65 (s, 2H), 8.27 (d, J=2.4 Hz, 1H), 8.12 (s, 2H), 7.65 (s, 1H), 7.43 (s, 1H), 6.57 (d, J=4.8 Hz, 1H), 4.53 (t, J=8.4 Hz, 1H), 3.76 (s, 3H), 2.88-2.93 (m, 1H), 2.50-2.52 (m, 1H), 2.21 (s, 3H), 1.96 (s, 3H). LC-MS: m/z 473 [M+H]⁺.

Example 57: ¹H NMR (400 MHz, DMSO-d₆) δ10.82 (s, 1H), 8.65 (s, 2H), 8.27 (d, J=2.4 Hz, 1H), 8.12 (s, 2H), 7.65 (s, 1H), 7.43 (s, 1H), 6.57 (d, J=4.8 Hz, 1H), 4.53 (t, J=8.4 Hz, 1H), 3.76 (s, 3H), 2.88-2.93 (m, 1H), 2.50-2.52 (m, 1H), 2.21 (s, 3H), 1.96 (s, 3H). LC-MS: m/z 473 [M+H]⁺.

### Step 5: Separation of Enantiomers to Obtain Example 58 and Example 59

22 mg of cis-2-fluoro-8-methyl-8-(1-methyl-1H-pyrazol-4-yl)-N-(5-methyl-6-(2H-1,2,3-triazol-2-yl)pyridin-3-yl)-7,8-dihydro-6H-cyclopenta[e]pyrazolo[1,5-a]pyrimidine-6-carboxamide were submitted to chiral HPLC purification (Column: CHIRALPAK IF, 2*25 cm, 5 m; Mobile Phase A: MtBE (0.5% 2M NH3-MeOH)-HPLC, Mobile Phase B: EtOH-HPLC; Flow rate: 17 mL/min; Gradient: isocratic 50% Mobile Phase B; Wave Length: 220/254 nm; RT1 (min): 7.783; RT2 (min): 16.654; Sample Solvent: EtOH-HPLC; Injection Volume: 2 mL; Number Of Runs: 1). The second eluting isomer was concentrated and lyophilized to afford Example 58 (5.0 mg, 22% yield) as a white solid. The first eluting isomer was concentrated and lyophilized to afford Example 59 (3.2 mg, 14% yield) as a white solid.

Example 58: ¹H NMR (400 MHz, DMSO-d₆) δ10.83 (s, 1H), 8.69 (s, 1H), 8.63 (d, J=2.4 Hz, 1H), 8.24 (d, J=2.4 Hz, 1H), 8.11 (s, 2H), 7.62 (s, 1H), 7.34 (s, 1H), 6.57 (d, J=4.9 Hz, 1H), 4.49-4.53 (m, 1H), 3.75 (s, 3H), 2.79-2.85 (m, 1H), 2.66-2.71 (m, 1H), 2.20 (s, 3H), 1.87 (s, 3H). LC-MS: m/z 473 [M+H]⁺.

Example 59: ¹H NMR (400 MHz, DMSO-d₆) δ10.82 (s, 1H), 8.69 (s, 1H), 8.63 (d, J=2.4 Hz, 1H), 8.24 (d, J=2.4 Hz, 1H), 8.11 (s, 2H), 7.62 (s, 1H), 7.34 (s, 1H), 6.57 (d, J=4.9 Hz, 1H), 4.49-4.53 (m, 1H), 3.75 (s, 3H), 2.79-2.85 (m, 1H), 2.66-2.71 (m, 1H), 2.20 (s, 3H), 1.87 (s, 3H). LC-MS: m/z 473 [M+H]⁺.

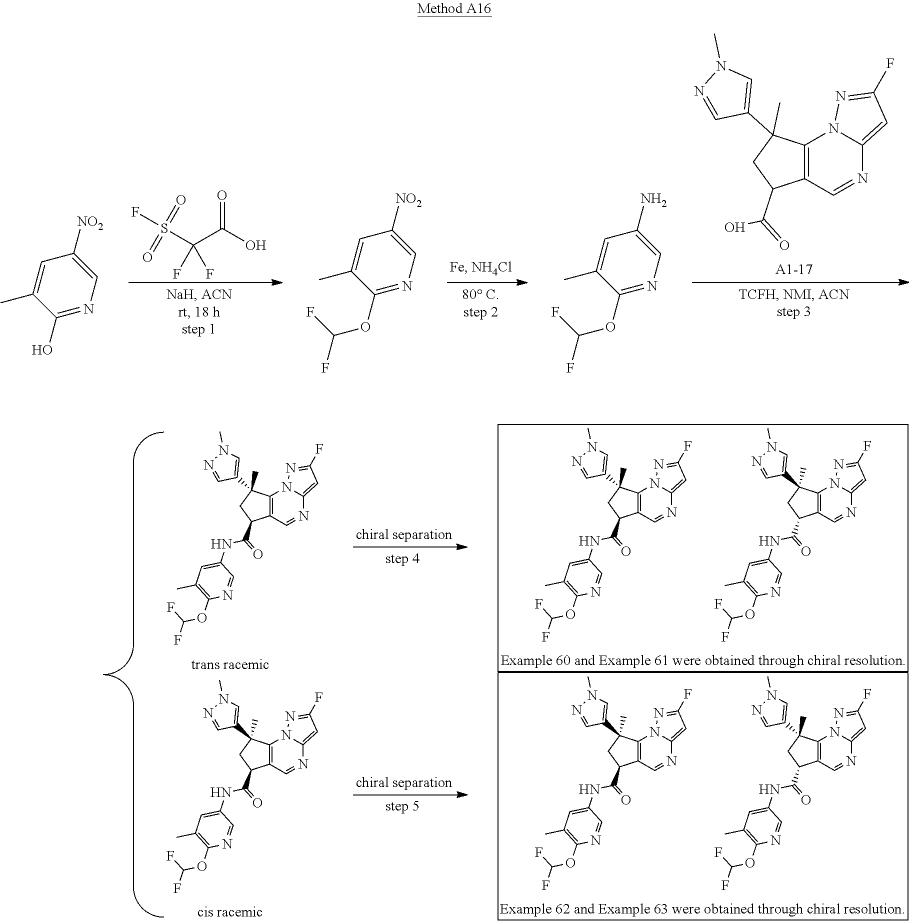

### Examples 60, 61: (6S,8R)—N-(6-(difluoromethoxy)-5-methylpyridin-3-yl)-2-fluoro-8-methyl-8-(1-methyl-1H-pyrazol-4-yl)-7,8-dihydro-6H-cyclopenta[e]pyrazolo[1,5-a]pyrimidine-6-carboxamide; (6R,8S)—N-(6-(difluoromethoxy)-5-methylpyridin-3-yl)-2-fluoro-8-methyl-8-(1-methyl-1H-pyrazol-4-yl)-7,8-dihydro-6H-cyclopenta[e]pyrazolo[1,5-a]pyrimidine-6-carboxamide

### Examples 62, 63: (6S,8S)—N-(6-(difluoromethoxy)-5-methylpyridin-3-yl)-2-fluoro-8-methyl-8-(1-methyl-1H-pyrazol-4-yl)-7,8-dihydro-6H-cyclopenta[e]pyrazolo[1,5-a]pyrimidine-6-carboxamide; (6R,8R)—N-(6-(difluoromethoxy)-5-methylpyridin-3-yl)-2-fluoro-8-methyl-8-(1-methyl-1H-pyrazol-4-yl)-7,8-dihydro-6H-cyclopenta[e]pyrazolo[1,5-a]pyrimidine-6-carboxamide

The relative and absolute stereochemistry of each example was not determined.

### Step 1: 2-(difluoromethoxy)-3-methyl-5-nitropyridine

To a stirred solution of 3-methyl-5-nitropyridin-2-ol (1 g, 6.5 mmol) in ACN (20 mL) was added NaH (700 mg, 17.5 mmol, 60% in mineral oil) in portions at 0° C. The mixture was stirred at 25° C. for 1h. 2,2-difluoro-2-(fluorosulfonyl)acetic acid (2.1 g, 11.7 mmol) was added dropwise at 0° C., and the resulting mixture was stirred at 25° C. for 18 h. The reaction was quenched with water (20 mL), and the resulting mixture was extracted with ethyl acetate (3×20 mL). The combined organic layers were washed with brine (60 mL), dried over anhydrous sodium sulfate and concentrated under reduced pressure. The residue was applied onto a silica gel column and eluted with EtOAc/PE (1:5) to give the title compound (800 mg, 60% yield) as a yellow oil. ¹H NMR (300 MHz, Chloroform-d) δ 8.92 (d, J=2.7 Hz, 1H), 8.36-8.34 (m, 1H), 7.55 (t, J=71.7 Hz, 1H), 2.39 (s, 3H). LC-MS (ES, m/z): 205 [M+H]⁺.

### Step 2: 6-(difluoromethoxy)-5-methylpyridin-3-amine

To a stirred solution of 2-(difluoromethoxy)-3-methyl-5-nitropyridine (700 mg, 3.4 mmol) in EtOH (15 mL) and water (5 mL) was added Fe (1.2 g, 20.6 mmol) and NH₄Cl (366.9 mg, 6.9 mmol). The reaction mixture was stirred at 80° C. for 2 h. The reaction mixture was filtered, and the filtrate was concentrated under reduced pressure. The residue was applied onto a silica gel column and eluted with EtOAc/PE (1:3) to give the title compound (500 mg, 83% yield) as a yellow oil. LC-MS: m/z 175 [M+H]⁺.

### Step 3: trans-N-(6-(difluoromethoxy)-5-methylpyridin-3-yl)-2-fluoro-8-methyl-8-(1-methyl-1H-pyrazol-4-yl)-7,8-dihydro-6H-cyclopenta[e]pyrazolo[1,5-a]pyrimidine-6-carboxamide and cis-N-(6-(difluoromethoxy)-5-methylpyridin-3-yl)-2-fluoro-8-methyl-8-(1-methyl-1H-pyrazol-4-yl)-7,8-dihydro-6H-cyclopenta[e]pyrazolo[1,5-a]pyrimidine-6-carboxamide

In analogy to Method A1 step 8 but using 2-fluoro-8-methyl-8-(1-methyl-1H-pyrazol-4-yl)-7,8-dihydro-6H-cyclopenta[e]pyrazolo[1,5-a]pyrimidine-6-carboxylic acid (A1-7, 70 mg, 222 μmol) and 6-(difluoromethoxy)-5-methylpyridin-3-amine (58 mg, 333 μmol) and stirring at 25° C. for 4 h, racemic mixtures of the title compounds were obtained: trans isomer (45 mg, 42% yield) as a white solid, LC-MS: m/z 472 [M+H]⁺; cis-isomer (25 mg, 23% yield) as a light pink solid, LC-MS: m/z 472 [M+H]⁺.

### Step 4: Separation of Enantiomers to Obtain Example 60 and Example 61

30 mg of trans-N-(6-(difluoromethoxy)-5-methylpyridin-3-yl)-2-fluoro-8-methyl-8-(1-methyl-1H-pyrazol-4-yl)-7,8-dihydro-6H-cyclopenta[e]pyrazolo[1,5-a]pyrimidine-6-carboxamide were submitted to chiral HPLC purification (Column: CHIRAL ART Cellulose-SC, 2*25 cm, 5 m; Mobile Phase A: Hex: DCM=3:1(0.1% FA)-HPLC, Mobile Phase B: IPA-HPLC; Flow rate: 20 mL/min; Gradient: isocratic 10% Mobile Phase B; Wave Length: 220/254 nm; RT1 (min): 5.792; RT2 (min): 8.757; Sample Solvent: EtOH-HPLC; Injection Volume: 0.6 mL; Number Of Runs: 3). The second eluting isomer was concentrated and lyophilized to afford Example 60 (7.1 mg, 23% yield) as a white solid. The first eluting isomer was concentrated and lyophilized to afford Example 61 (9.0 mg, 29% yield) as a white solid.

Example 60: ¹HNMR (400 MHz, DMSO-d₆) δ10.51 (s, 1H), 8.59 (s, 1H), 8.30 (d, J=2.4 Hz, 1H), 8.03 (d, J=2.0 Hz, 1H), 7.66 (t, J=73.2 Hz, 1H), 7.63 (s, 1H), 7.41 (s, 1H), 6.55 (d, J=4.8 Hz, 111), 4.43-4.48 (m, 111), 3.75 (s, 3H), 2.83-2.89 (m, 111), 2.44-2.52 (m, 1H), 2.22 (s, 311), 1.95 (s, 3H). LC-MS: m/z 472 [M+H]⁺.

Example 61: ¹HNMR (400 MHz, DMSO-d₆) δ10.51 (s, 1H), 8.59 (s, 1H), 8.30 (d, J=2.4 Hz, 1H), 8.03 (d, J=2.0 Hz, 1H), 7.66 (t, J=73.2 Hz, 1H), 7.63 (s, 1H), 7.41 (s, 1H), 6.55 (d, J=4.8 Hz, 1H), 4.43-4.48 (m, 1H), 3.75 (s, 3H), 2.83-2.89 (m, 1H), 2.44-2.52 (m, 1H), 2.22 (s, 3H), 1.95 (s, 3H). LC-MS: m/z 472 [M+H]⁺.

### Step 5: Separation of Enantiomers to Obtain Example 62 and Example 63

20 mg of cis-N-(6-(difluoromethoxy)-5-methylpyridin-3-yl)-2-fluoro-8-methyl-8-(1-methyl-1H-pyrazol-4-yl)-7,8-dihydro-6H-cyclopenta[e]pyrazolo[1,5-a]pyrimidine-6-carboxamide were submitted to chiral HPLC purification (Column: CHIRALPAK IG, 2*25 cm, 5 m; Mobile Phase A: Hex: DCM=3:1(0.5% 2M NH3-MeOH)-HPLC, Mobile Phase B: EtOH-HPLC; Flow rate: 20 mL/min; Gradient: isocratic 10% Mobile Phase B; Wave Length: 220/254 nm; RT1 (min): 9.608; RT2 (min): 11.131; Sample Solvent: EtOH-HPLC; Injection Volume: 0.3 mL; Number Of Runs: 4). The first eluting isomer was concentrated and lyophilized to afford Example 62 (2.8 mg, 13% yield) as a white solid. The second eluting isomer was concentrated and lyophilized to afford Example 63 (2.6 mg, 12% yield) as a white solid.

Example 62: ¹HNMR (400 MHz, DMSO-d₆) δ10.51 (s, 1H), 8.65 (s, 1H), 8.28 (d, J=2.4 Hz, 1H), 8.01 (d, J=2.0 Hz, 1H), 7.65 (t, J=73.2 Hz, 1H), 7.61 (s, 1H), 7.33 (s, 1H), 6.53 (d, J=4.8 Hz, 1H), 4.42-4.47 (m, 1H), 3.75 (s, 3H), 2.75-2.81 (m, 1H), 2.63-2.68 (m, 1H), 2.21 (s, 3H), 1.85 (s, 3H). LC-MS: m/z 472 [M+H]⁺.

Example 63: ¹HNMR (400 MHz, DMSO-d₆) δ10.51 (s, 1H), 8.65 (s, 1H), 8.28 (d, J=2.4 Hz, 1H), 8.01 (d, J=2.0 Hz, 1H), 7.65 (t, J=73.2 Hz, 1H), 7.61 (s, 1H), 7.33 (s, 1H), 6.53 (d, J=4.8 Hz, 1H), 4.42-4.47 (m, 1H), 3.75 (s, 3H), 2.75-2.81 (m, 1H), 2.63-2.68 (m, 1H), 2.21 (s, 3H), 1.85 (s, 3H). LC-MS: m/z 472 [M+H]⁺.

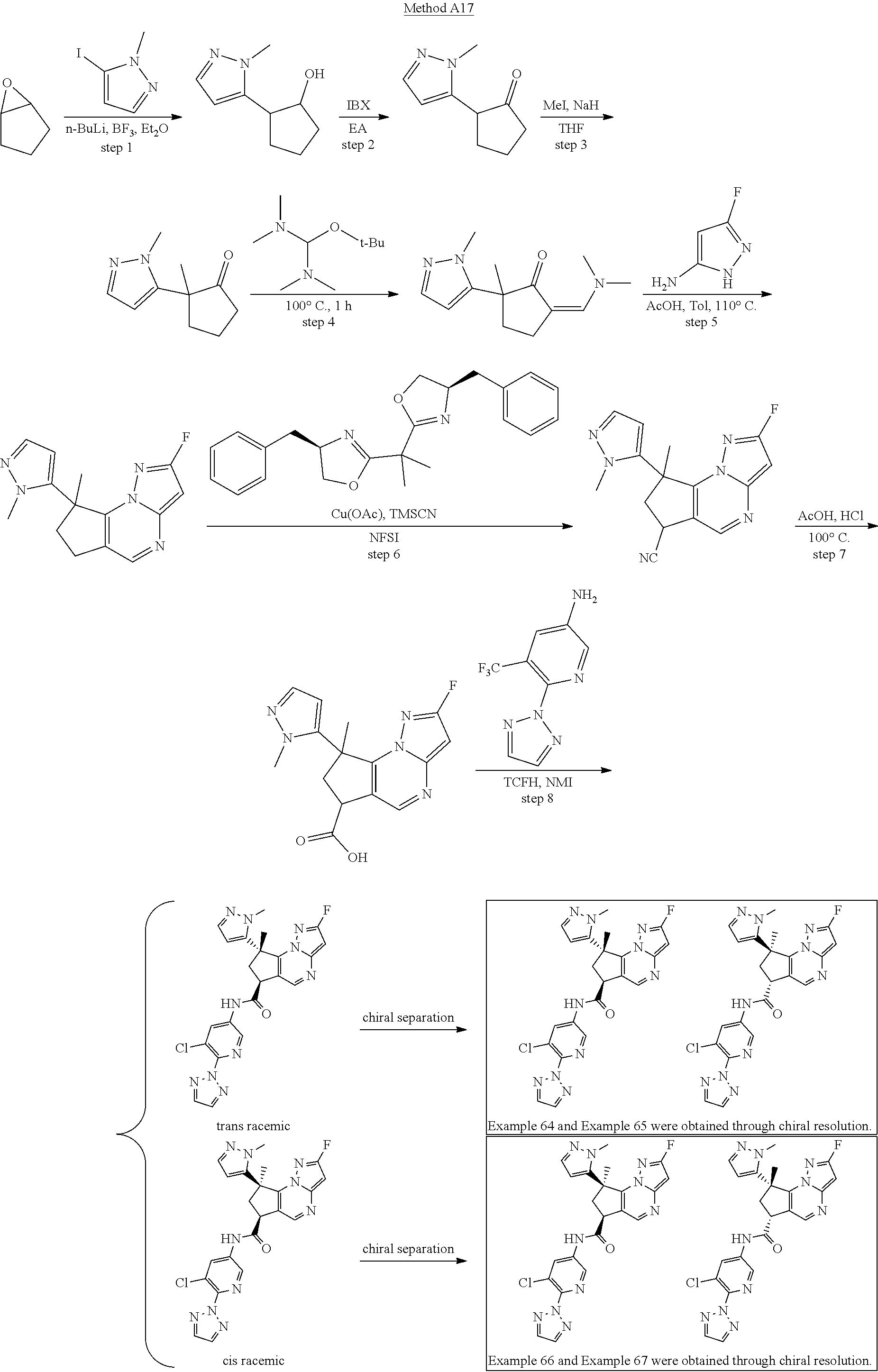

### Examples 64, 65: (6S,8R)—N-(5-chloro-6-(2H-1,2,3-triazol-2-yl)pyridin-3-yl)-2-fluoro-8-methyl-8-(1-methyl-1H-pyrazol-5-yl)-7,8-dihydro-6H-cyclopenta[e]pyrazolo[1,5-a]pyrimidine-6-carboxamide; (6R,8S)—N-(5-chloro-6-(2H-1,2,3-triazol-2-yl)pyridin-3-yl)-2-fluoro-8-methyl-8-(1-methyl-1H-pyrazol-5-yl)-7,8-dihydro-6H-cyclopenta[e]pyrazolo[1,5-a]pyrimidine-6-carboxamide

### Examples 66, 67: (6S,8S)—N-(5-chloro-6-(2H-1,2,3-triazol-2-yl)pyridin-3-yl)-2-fluoro-8-methyl-8-(1-methyl-1H-pyrazol-5-yl)-7,8-dihydro-6H-cyclopenta[e]pyrazolo[1,5-a]pyrimidine-6-carboxamide; (6R,8R)—N-(5-chloro-6-(2H-1,2,3-triazol-2-yl)pyridin-3-yl)-2-fluoro-8-methyl-8-(1-methyl-1H-pyrazol-5-yl)-7,8-dihydro-6H-cyclopenta[e]pyrazolo[1,5-a]pyrimidine-6-carboxamide

The relative and absolute stereochemistry of each example was not determined.

### Step 1: 2-(1-methyl-1H-pyrazol-5-yl)cyclopentan-1-ol

In analogy to Method A6 step 2 but using 5-iodo-1-methyl-1H-pyrazole (10 g, 48.0 mmol), the title compound was obtained as a yellow oil (3.7 g, 46% yield). ¹H NMR (400 MHz, Chloroform-d) δ 7.38 (d, J=2.0 Hz, 1H), 6.03 (d, J=2.0 Hz, 1H), 4.23 (q, J=6.4 Hz, 1H), 3.85 (s, 3H), 3.02 (m, 1H), 2.27 (m, 2H), 1.94-1.59 (m, 4H). LC-MS: m/z 167 [M+H]⁺.

### Step 2: 2-(1-methyl-1H-pyrazol-5-yl)cyclopentan-1-one

In analogy to Method A10 step 2 but using 2-(1-methyl-1H-pyrazol-5-yl)cyclopentan-1-ol (7.5 g, 45.1 mmol), stirring for 16 h and purification of the product by silica gel column chromatography eluting with EtOAc/PE (3:2), the title compound was obtained as a yellow oil (6 g, 80% yield). LC-MS: m/z 165 [M+H]⁺.

### Step 3: 2-methyl-2-(1-methyl-1H-pyrazol-5-yl)cyclopentan-1-one

To a stirred solution of 2-(1-methyl-1H-pyrazol-5-yl)cyclopentan-1-one (4.6 g, 28.0 mmol) in THF (20 mL) was added NaH (1.3 g, 56.0 mmol, 60% in mineral oil) in portions at 0° C. The mixture was stirred at 0° C. for 10 min. Iodomethane (2.7 g, 19.6 mmol) was added dropwise at 0° C., and the resulting mixture was stirred at 80° C. for 16 h. The reaction was quenched with water (50 mL), and the resulting mixture was extracted with ethyl acetate (3×50 mL). The combined organic layers were dried over anhydrous sodium sulfate and concentrated under reduced pressure. The residue was applied onto a silica gel column and eluted with EtOAc/PE (3:2) to afford the title compound (4 g, 80% yield) as a yellow oil. LC-MS: m/z 179 [M+H]⁺.

### Step 4: 5-((dimethylamino)methylene)-2-methyl-2-(1-methyl-1H-pyrazol-5-yl)cyclopentan-1-one

In analogy to Method A1 step 4 but using 2-methyl-2-(1-methyl-1H-pyrazol-5-yl)cyclopentan-1-one (4.5 g, 25.2 mmol) in 1-tert-butoxy-N,N,N′,N′-tetramethyl-methanediamine (8.8 g, 50.5 mmol) and stirring stirred at 100° C. for 16 h, the crude title compound was obtained as a red oil (5.5 g, 93% yield). LC-MS: m/z 234 [M+H]⁺.

### Step 5: 2-fluoro-8-methyl-8-(1-methyl-1H-pyrazol-5-yl)-7,8-dihydro-6H-cyclopenta[e]pyrazolo[1,5-a]pyrimidine

In analogy to Method A1 step 5 but using 5-((dimethylamino)methylene)-2-methyl-2-(1-methyl-1H-pyrazol-5-yl)cyclopentan-1-one (5.5 g, 23.5 mmol), the title compound was obtained as a yellow solid (1.8 g, 28% yield). LC-MS: m/z 272[M+H]⁺.

### Step 6: 2-fluoro-8-methyl-8-(1-methyl-1H-pyrazol-5-yl)-7,8-dihydro-6H-cyclopenta[e]pyrazolo[1,5-a]pyrimidine-6-carbonitrile

In analogy to Method A1 step 6 but using 2-fluoro-8-methyl-8-(1-methyl-1H-pyrazol-5-yl)-7,8-dihydro-6H-cyclopenta[e]pyrazolo[1,5-a]pyrimidine (1.5 g, 5.5 mmol) in chlorobenzene (150 mL), the title compound was obtained as a yellow solid (1.5 g, 91% yield). LC-MS: m/z 297 [M+H]⁺.

### Step 7: 2-fluoro-8-methyl-8-(1-methyl-1H-pyrazol-5-yl)-7,8-dihydro-6H-cyclopenta[e]pyrazolo[1,5-a]pyrimidine-6-carboxylic acid

In analogy to Method A1 step 7 but using 2-fluoro-8-methyl-8-(1-methyl-1H-pyrazol-5-yl)-7,8-dihydro-6H-cyclopenta[e]pyrazolo[1,5-a]pyrimidine-6-carbonitrile (1.5 g, 5.0 mmol), stirring at 100° C. for 3 h and purification of the product by prep-HPLC, the title compound was obtained as a yellow solid (380 mg, 23% yield). LC-MS: m/z 316 [M+H]⁺.

### Step 8: trans-N-(5-chloro-6-(2H-1,2,3-triazol-2-yl)pyridin-3-yl)-2-fluoro-8-methyl-8-(1-methyl-1H-pyrazol-5-yl)-7,8-dihydro-6H-cyclopenta[e]pyrazolo[1,5-a]pyrimidine-6-carboxamide and cis-N-(5-chloro-6-(2H-1,2,3-triazol-2-yl)pyridin-3-yl)-2-fluoro-8-methyl-8-(1-methyl-1H-pyrazol-5-yl)-7,8-dihydro-6H-cyclopenta[e]pyrazolo[1,5-a]pyrimidine-6-carboxamide

In analogy to Method A1 step 8 but using 2-fluoro-8-methyl-8-(1-methyl-1H-pyrazol-5-yl)-7,8-dihydro-6H-cyclopenta[c]pyrazolo[1,5-a]pyrimidine-6-carboxylic acid (200 mg, 634 μmol) and 5-chloro-6-(triazol-2-yl)pyridin-3-amine (124 mg, 634 μmol), racemic mixtures of the title compounds were obtained: trans isomer (50 mg, 31% yield) as a white solid, LC-MS: m/z 493 [M+H]⁺; cis-isomer (70 mg, 44% yield) as a white solid, LC-MS: m/z 493 [M+H]⁺.

### Step 9: Separation of Enantiomers to Obtain Example 64 and Example 65

50 mg of trans-N-(5-chloro-6-(2H-1,2,3-triazol-2-yl)pyridin-3-yl)-2-fluoro-8-methyl-8-(1-methyl-1H-pyrazol-5-yl)-7,8-dihydro-6H-cyclopenta[e]pyrazolo[1,5-a]pyrimidine-6-carboxamide were submitted to chiral HPLC purification (Column: CHIRALPAK IF, 2*25 cm, 5 m; Mobile Phase A: Hex: DCM=3:1(0.1% FA)-HPLC, Mobile Phase B: EtOH-HPLC; Flow rate: 20 mL/min; Gradient: isocratic 30% Mobile Phase B; Wave Length: 254/220 nm; RT1 (min): 7.592; RT2 (min): 11.79; Sample Solvent: EtOH-HPLC; Injection Volume: 1.5 mL; Number Of Runs: 4). The second eluting isomer was concentrated and lyophilized to afford Example 64 (21.2 mg, 83% yield) as a white solid. The first eluting isomer was concentrated and lyophilized to afford Example 65 (17.0 mg, 66% yield) as a white solid.

Example 64: ¹H NMR (400 MHz, DMSO-d₆) δ 11.18 (s, 1H), 8.79 (s, 1H), 8.73 (d, J=2.0 Hz, 1H), 8.58 (d, J=2.0 Hz, 1H), 8.17 (s, 2H), 7.32 (d, J=2.0 Hz, 1H), 6.63 (d, J=4.8 Hz, 1H), 6.04 (d, J=2.0 Hz, 1H), 4.56-4.60 (m, 1H), 3.46 (s, 3H), 2.93-2.99 (m, 1H), 2.62-2.67 (m, 1H), 2.00 (s, 3H). LC-MS: m/z 493 [M+H]⁺.

Example 65: ¹H NMR (400 MHz, DMSO-d₆) δ11.16 (s, 1H), 8.79 (s, 1H), 8.73 (d, J=2.0 Hz, 1H), 8.58 (d, J=2.0 Hz, 1H), 8.17 (s, 2H), 7.32 (d, J=2.0 Hz, 1H), 6.63 (d, J=4.8 Hz, 1H), 6.04 (d, J=2.0 Hz, 1H), 4.56-4.60 (m, 1H), 3.46 (s, 3H), 2.93-2.99 (m, 1H), 2.62-2.67 (m, 1H), 2.00 (s, 3H). LC-MS: m/z 493 [M+H]⁺.

### Step 10: Separation of Enantiomers to Obtain Example 66 and Example 67

70 mg of cis-N-(5-chloro-6-(2H-1,2,3-triazol-2-yl)pyridin-3-yl)-2-fluoro-8-methyl-8-(1-methyl-1H-pyrazol-5-yl)-7,8-dihydro-6H-cyclopenta[e]pyrazolo[1,5-a]pyrimidine-6-carboxamide was submitted to chiral HPLC purification (Column: CHIRALPAK IF 2*25 cm, 5 μm; Mobile Phase A: Hex: DCM=3:1(0.1% FA)-HPLC, Mobile Phase B: EtOH-HPLC; Flow rate: 20 mL/min; Gradient: isocratic 30% Mobile Phase B; Wave Length: 254/220 nm; RT1 (min): 6.883; RT2 (min): 9.78; Sample Solvent: EtOH-HPLC; Injection Volume: 1 mL; Number Of Runs: 5). The second eluting isomer was concentrated and lyophilized to afford Example 66 (20.7 mg, 59% yield) as a white solid. The first eluting isomer was concentrated and lyophilized to afford Example 67 (15.8 mg, 45% yield) as a white solid.

Example 66: ¹H NMR (400 MHz, DMSO-d₆) δ 11.22 (s, 1H), 8.76 (s, 1H), 8.73 (d, J=2.4 Hz, 111), 8.62 (d, J=2.4 Hz, 111), 8.17 (s, 211), 7.38 (d, J=2.0 Hz, 1H), 6.61 (d, J=4.8 Hz, 111), 6.38 (d, J=2.0 Hz, 1H), 4.63 (t, J=8.4 Hz, 1H), 3.23 (s, 3H), 2.72-2.81 (m, 2H), 1.89 (s, 3H). LC-MS: m/z 493 [M+H]⁺.

Example 67: ¹H NMR (400 MHz, DMSO-d₆) δ 11.22 (s, 1H), 8.76 (s, 1H), 8.73 (d, J=2.4 Hz, 1H), 8.62 (d, J=2.4 Hz, 1H), 8.17 (s, 2H), 7.38 (d, J=2.0 Hz, 1H), 6.61 (d, J=4.8 Hz, 1H), 6.38 (d, J=2.0 Hz, 1H), 4.63 (t, J=8.4 Hz, 1H), 3.23 (s, 3H), 2.72-2.81 (m, 2H), 1.89 (s, 3H). LC-MS: m/z 493 [M+H]⁺.

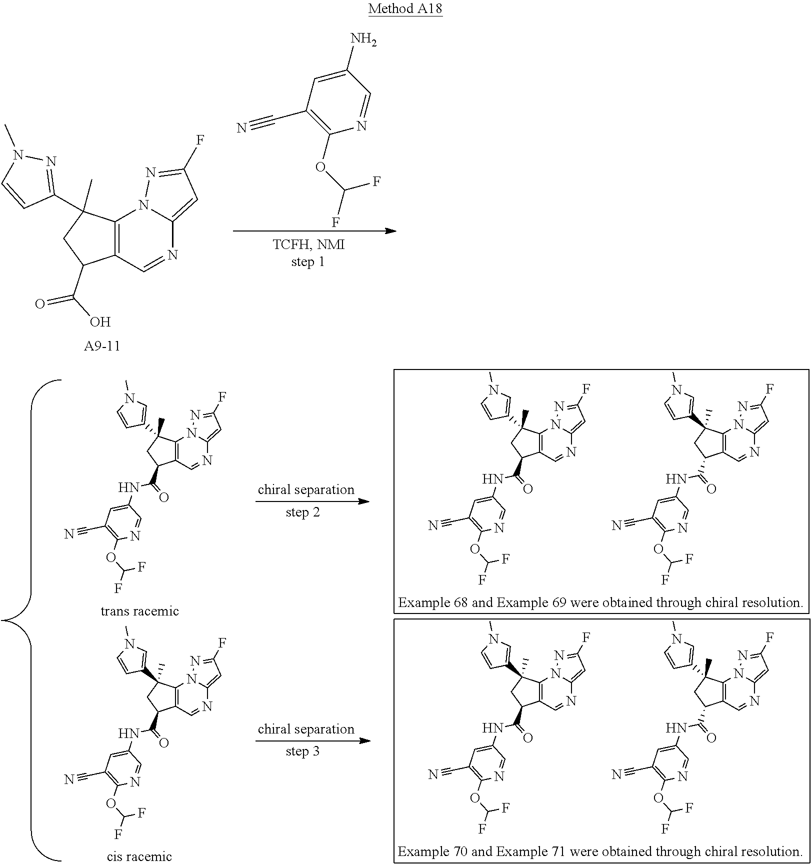

### Examples 68, 69: (6S,8S)—N-(5-cyano-6-(difluoromethoxy)pyridin-3-yl)-2-fluoro-8-methyl-8-(1-methyl-1H-pyrazol-3-yl)-7,8-dihydro-6H-cyclopenta[e]pyrazolo[1,5-a]pyrimidine-6-carboxamide; (6R,8R)—N-(5-cyano-6-(difluoromethoxy)pyridin-3-yl)-2-fluoro-8-methyl-8-(1-methyl-1H-pyrazol-3-yl)-7,8-dihydro-6H-cyclopenta[e]pyrazolo[1,5-a]pyrimidine-6-carboxamide

### Examples 70, 71: (6S,8R)—N-(5-cyano-6-(difluoromethoxy)pyridin-3-yl)-2-fluoro-8-methyl-8-(1-methyl-1H-pyrazol-3-yl)-7,8-dihydro-6H-cyclopenta[e]pyrazolo[1,5-a]pyrimidine-6-carboxamide; (6R,8S)—N-(5-cyano-6-(difluoromethoxy)pyridin-3-yl)-2-fluoro-8-methyl-8-(1-methyl-1H-pyrazol-3-yl)-7,8-dihydro-6H-cyclopenta[e]pyrazolo[1,5-a]pyrimidine-6-carboxamide

The relative and absolute stereochemistry of each example was not determined.

### Step 1: trans-N-(5-cyano-6-(difluoromethoxy)pyridin-3-yl)-2-fluoro-8-methyl-8-(1-methyl-1H-pyrazol-3-yl)-7,8-dihydro-6H-cyclopenta[e]pyrazolo[1,5-a]pyrimidine-6-carboxamide and cis-N-(5-cyano-6-(difluoromethoxy)pyridin-3-yl)-2-fluoro-8-methyl-8-(1-methyl-1H-pyrazol-3-yl)-7,8-dihydro-6H-cyclopenta[e]pyrazolo[1,5-a]pyrimidine-6-carboxamide

In analogy to Method A1 step 8 but using 2-fluoro-8-methyl-8-(1-methyl-1H-pyrazol-3-yl)-7,8-dihydro-6H-cyclopenta[e]pyrazolo[1,5-a]pyrimidine-6-carboxylic acid (A9-11, 80 mg, 254 μmol) and 5-amino-2-(difluoromethoxy)pyridine-3-carbonitrile (94 mg, 507 μmol), racemic mixtures of the title compounds were obtained: trans isomer (15 mg, 11% yield) as a white solid, LC-MS: m/z 483 [M+H]⁺; cis-isomer (40 mg, 32% yield) as a white solid, LC-MS: m/z 483 [M+H]⁺.

### Step 2: Separation of Enantiomers to Obtain Example 68 and Example 69

40 mg of trans-N-(5-cyano-6-(difluoromethoxy)pyridin-3-yl)-2-fluoro-8-methyl-8-(1-methyl-1H-pyrazol-3-yl)-7,8-dihydro-6H-cyclopenta[e]pyrazolo[1,5-a]pyrimidine-6-carboxamide were submitted to chiral HPLC purification (Column: CHIRALPAK IE 2*25 cm, 5 m; Mobile Phase A: Hex: DCM=1:1(0.1% FA)-HPLC, Mobile Phase B: EtOH-HPLC; Flow rate: 20 mL/min; Gradient: isocratic 10% Mobile Phase B; Wave Length: 254/220 nm; RT1 (min): 5.063; RT2 (min): 6.557; Sample Solvent: EtOH-HPLC; Injection Volume: 0.5 mL; Number Of Runs: 4). The second eluting isomer was concentrated and lyophilized to afford Example 68 (8.0 mg, 19% yield) as a white solid. The first eluting isomer was concentrated and lyophilized to afford Example 69 (10.7 mg, 26% yield) as a white solid.

Example 68: ¹H NMR (400 MHz, Chloroform-d) δ 8.59 (d, J=2.4 Hz, 1H), 8.54-8.51 (m, 2H), 8.35 (s, 1H), 7.45 (t, J=71 Hz, 1H), 7.32 (d, J=2.4 Hz, 1H), 6.64 (d, J=1.6 Hz, 1H), 6.25 (d, J=5.2 Hz, 1H), 4.61 (t, J=8.4 Hz, 1H), 3.89 (s, 3H), 3.44-3.49 (m, 1H), 2.48-2.54 (m, 1H), 2.11 (s, 3H). LC-MS: m/z 483 [M+H]⁺.

Example 69: ¹H NMR (400 MHz, Chloroform-d) δ 8.60 (d, J=2.4 Hz, 1H), 8.54-8.51 (m, 2H), 8.39 (s, 1H), 7.45 (t, J=72 Hz, 1H), 7.33 (d, J=2 Hz, 1H), 6.65 (d, J=2 Hz, 1H), 6.25 (d, J=5.2 Hz, 1H), 4.63 (t, J=8.4 Hz, 1H), 3.90 (s, 3H), 3.45-3.50 (m, 1H), 2.48-2.54 (m, 1H), 2.09 (s, 3H). LC-MS: m/z 483 [M+H]⁺.

### Step 3: Separation of Enantiomers to Obtain Example 70 and Example 71

15 mg of cis-(6S,8R)—N-(5-cyano-6-(difluoromethoxy)pyridin-3-yl)-2-fluoro-8-methyl-8-(1-methyl-1H-pyrazol-3-yl)-7,8-dihydro-6H-cyclopenta[e]pyrazolo[1,5-a]pyrimidine-6-carboxamide were submitted to chiral HPLC purification (Column: CHIRAL ART Cellulose-SC 2*25 cm, 5 μm; Mobile Phase A: Hex: DCM=3:1(0.1% FA)-HPLC, Mobile Phase B: EtOH-HPLC; Flow rate: 20 mL/min; Gradient: isocratic 20% Mobile Phase B; Wave Length: 254/220 nm; RT1 (min): 8.977; RT2 (min): 14.167; Sample Solvent: EtOH-HPLC; Injection Volume: 1 mL; Number Of Runs: 4). The second eluting isomer was concentrated and lyophilized to afford Example 70 (4.7 mg, 31% yield) as a white solid. The first eluting isomer was concentrated and lyophilized to afford Example 71 (3.9 mg, 25% yield) as a white solid.

Example 70: ¹H NMR (400 MHz, Chloroform-d) δ 10.65 (s, 1H), 8.57 (s, 1H), 8.47 (s, 2H), 7.42 (t, 72 Hz, 1H), 7.41 (s, 1H), 6.73 (d, J=2 Hz, 1H), 6.26 (d, J=5.2 Hz, 1H), 4.47-4.51 (m, 1H), 3.83 (s, 3H), 3.16-3.21 (m, 1H), 2.81-2.87 (m, 1H), 2.07 (s, 3H). LC-MS: m/z 483 [M+H]⁺.

Example 71: ¹H NMR (400 MHz, Chloroform-d) δ 10.65 (s, 1H), 8.57 (s, 1H), 8.48 (s, 2H), 7.43 (t, J=68 Hz, 1H), 7.42 (s, 1H), 6.74 (d, J=2.4 Hz, 1H), 6.27 (d, J=4.8 Hz, 1H), 4.48-4.51 (m, 1H), 3.84 (s, 3H), 3.16-3.20 (m, 1H), 2.82-2.88 (m, 1H), 2.07 (s, 3H). LC-MS: m/z 483 [M+H]⁺.

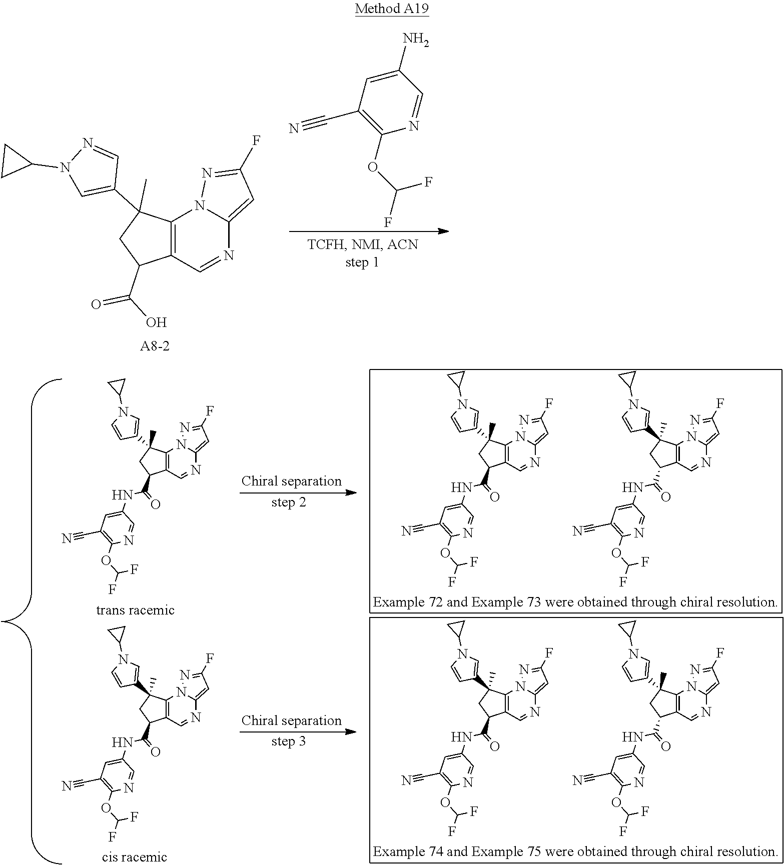

### Examples 72, 73: (6S,8R)—N-(5-cyano-6-(difluoromethoxy)pyridin-3-yl)-8-(1-cyclopropyl-1H-pyrazol-4-yl)-2-fluoro-8-methyl-7,8-dihydro-6H-cyclopenta[e]pyrazolo[1,5-a]pyrimidine-6-carboxamide; (6R,8S)—N-(5-cyano-6-(difluoromethoxy)pyridin-3-yl)-8-(1-cyclopropyl-1H-pyrazol-4-yl)-2-fluoro-8-methyl-7,8-dihydro-6H-cyclopenta[e]pyrazolo[1,5-a]pyrimidine-6-carboxamide

### Examples 74, 75: (6S,8S)—N-(5-cyano-6-(difluoromethoxy)pyridin-3-yl)-8-(1-cyclopropyl-1H-pyrazol-4-yl)-2-fluoro-8-methyl-7,8-dihydro-6H-cyclopenta[e]pyrazolo[1,5-a]pyrimidine-6-carboxamide; (6R,8R)—N-(5-cyano-6-(difluoromethoxy)pyridin-3-yl)-8-(1-cyclopropyl-1H-pyrazol-4-yl)-2-fluoro-8-methyl-7,8-dihydro-6H-cyclopenta[e]pyrazolo[1,5-a]pyrimidine-6-carboxamide

The relative and absolute stereochemistry of each example was not determined.

### Step 1: trans-N-(5-cyano-6-(difluoromethoxy)pyridin-3-yl)-8-(1-cyclopropyl-1H-pyrazol-4-yl)-2-fluoro-8-methyl-7,8-dihydro-6H-cyclopenta[e]pyrazolo[1,5-a]pyrimidine-6-carboxamide and cis-N-(5-cyano-6-(difluoromethoxy)pyridin-3-yl)-8-(1-cyclopropyl-1H-pyrazol-4-yl)-2-fluoro-8-methyl-7,8-dihydro-6H-cyclopenta[e]pyrazolo[1,5-a]pyrimidine-6-carboxamide

In analogy to Method A1 step 8 but using 8-(1-cyclopropyl-1H-pyrazol-4-yl)-2-fluoro-8-methyl-7,8-dihydro-6H-cyclopenta[e]pyrazolo[1,5-a]pyrimidine-6-carboxylic acid (A8-2, 50 mg, 146 mol) and 5-amino-2-(difluoromethoxy)pyridine-3-carbonitrile (27 mg, 146 mol), racemic mixtures of the title compounds were obtained: trans isomer (20 mg, 26% yield) as a white solid, LC-MS: m/z 509 [M+H]⁺; cis-isomer (10 mg, 13% yield) as a white solid, LC-MS: m/z 509 [M+H]⁺.

### Step 2: Separation of Enantiomers to Obtain Example 72 and Example 73

20 mg of trans-N-(5-cyano-6-(difluoromethoxy)pyridin-3-yl)-8-(1-cyclopropyl-11H-pyrazol-4-yl)-2-fluoro-8-methyl-7,8-dihydro-6H-cyclopenta[e]pyrazolo[1,5-a]pyrimidine-6-carboxamide were submitted to chiral HPLC purification (Column: CHIRALPAK IF, 2×25 cm, 5 m; Mobile Phase A: Hex: DCM=1:1(0.1% FA)-HPLC, Mobile Phase B: IPA-HPLC; Flow rate: 20 ML/MIN mL/min; Gradient: isocratic 10% Mobile Phase B; Wave Length: 254/220 nm nm; RT1 (min): 4.331; RT2 (min): 5.782; Sample Solvent: EtOH-HPLC; Injection Volume: 2.0 mL; Number Of Runs: 2.). The second eluting isomer was concentrated and lyophilized to afford Example 72 (9.3 mg, 37% yield) as a white solid. The first eluting isomer was concentrated and lyophilized to afford Example 73 (11.4 mg, 45% yield) as a white solid.

Example 72: ¹H NMR (400 MHz, Chloroform-d) δ 9.61 (s, 1H), 8.62-8.67 (m, 2H), 8.53 (s, 1H), 8.14 (s, 1H), 7.92 (s, 1H), 7.41 (t, J=71.6 Hz, 1H), 6.29 (d, J=4.8 Hz, 1H), 4.57-4.59 (m, 1H), 3.62-3.63 (m, 1H), 3.29-3.30 (m, 1H), 2.58-2.64 (m, 1H), 2.08 (s, 3H), 1.14-1.17 (m, 4H). LC-MS: m/z 509 [M+H]⁺.

Example 73: ¹H NMR (400 MHz, Chloroform-d) δ 9.32 (s, 1H), 8.60-8.64 (m, 2H), 8.52 (s, 1H), 7.94 (s, 1H), 7.83 (s, 1H), 7.42 (t, J=71.6 Hz, 1H), 6.29 (d, J=4.8 Hz, 1H), 4.52-4.54 (m, 1H), 3.57-3.59 (m, 1H), 3.18-3.20 (m, 1H), 2.60-2.65 (m, 1H), 2.07 (s, 3H), 1.09-1.13 (m, 4H). LC-MS: m/z 509 [M+H]⁺.

### Step 3: Separation of Enantiomers to Obtain Example 74 and Example 75

10 mg of cis-N-(5-cyano-6-(difluoromethoxy)pyridin-3-yl)-8-(1-cyclopropyl-1H-pyrazol-4-yl)-2-fluoro-8-methyl-7,8-dihydro-6H-cyclopenta[e]pyrazolo[1,5-a]pyrimidine-6-carboxamide were submitted to chiral HPLC purification (Column: CHIRALPAK IF, 2*25 cm, 5 μm; Mobile Phase A: Hex: DCM=1:1(0.1% FA)-HPLC, Mobile Phase B: IPA-HPLC; Flow rate: 20 ML/MIN mL/min; Gradient: isocratic 10% Mobile Phase B; Wave Length: 254/220 nm nm; RT1 (min): 6.881; RT2 (min): 16.445; Sample Solvent: EtOH-HPLC; Injection Volume: 2.0 mL; Number Of Runs: 2). The first eluting isomer was concentrated and lyophilized to afford Example 74 (3.2 mg, 31% yield) as a white solid. The first eluting isomer was concentrated and lyophilized to afford Example 75 (2.3 mg, 22% yield) as a white solid.

Example 74: ¹H NMR (400 MHz, Chloroform-d) δ 8.57 (s, 1H), 8.37 (s, 1H), 8.28 (s, 1H), 8.00 (s, 1H), 7.60-7.64 (m, 2H), 7.40 (t, J=71.6 Hz, 1H), 6.28 (d, J=5.2 Hz, 1H), 4.40-4.42 (m, 1H), 3.53-3.56 (m, 1H), 2.95-2.98 (m, 1H), 2.82-2.88 (m, 1H), 2.06 (s, 3H), 1.00-1.11 (m, 4H). LC-MS: m/z 509 [M+H]⁺.

Example 75: ¹H NMR (400 MHz, Chloroform-d) δ 8.62 (s, 1H), 8.55 (s, 11H), 8.38-8.42 (m, 2H), 7.70-7.74 (m, 2H), 7.39 (t, J=71.6 Hz, 1H), 6.27 (d, J=4.8 Hz, 1H), 4.48-4.50 (m, 1H), 3.59-3.60 (m, 1H), 2.86-2.99 (m, 2H), 1.98 (s, 3H), 1.08-1.12 (m, 4H). LC-MS: m/z 509 [M+H]⁺.

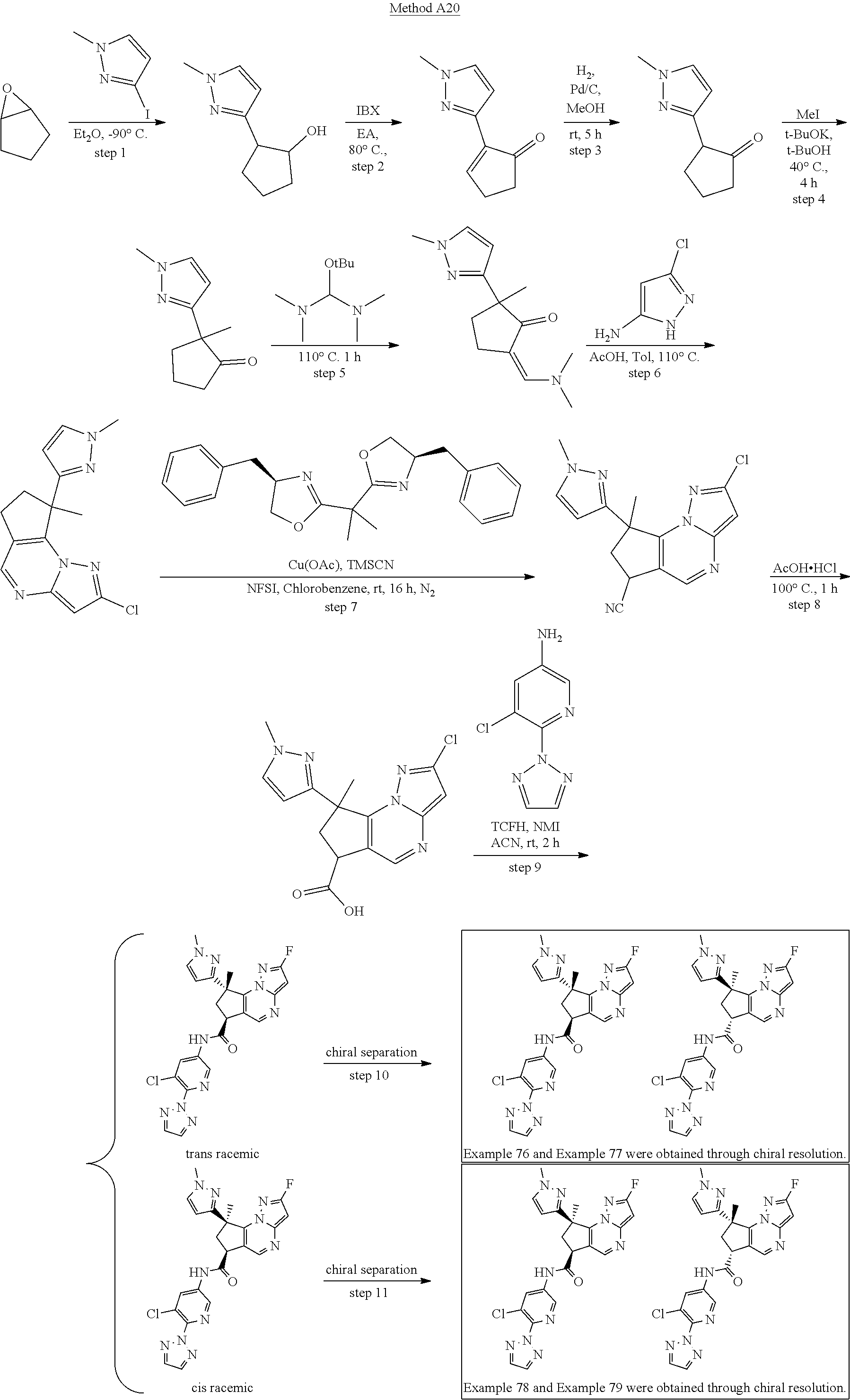

### Examples 76, 77: (6S,8S)-2-chloro-N-(5-chloro-6-(2H-1,2,3-triazol-2-yl)pyridin-3-yl)-8-methyl-8-(1-methyl-1H-pyrazol-3-yl)-7,8-dihydro-6H-cyclopenta[e]pyrazolo[1,5-a]pyrimidine-6-carboxamide; (6R,8R)-2-chloro-N-(5-chloro-6-(2H-1,2,3-triazol-2-yl)pyridin-3-yl)-8-methyl-8-(1-methyl-1H-pyrazol-3-yl)-7,8-dihydro-6H-cyclopenta[e]pyrazolo[1,5-a]pyrimidine-6-carboxamide

### Examples 78, 79: (6S,8R)-2-chloro-N-(5-chloro-6-(2H-1,2,3-triazol-2-yl)pyridin-3-yl)-8-methyl-8-(1-methyl-1H-pyrazol-3-yl)-7,8-dihydro-6H-cyclopenta[e]pyrazolo[1,5-a]pyrimidine-6-carboxamide; (6R,8S)-2-chloro-N-(5-chloro-6-(2H-1,2,3-triazol-2-yl)pyridin-3-yl)-8-methyl-8-(1-methyl-1H-pyrazol-3-yl)-7,8-dihydro-6H-cyclopenta[e]pyrazolo[1,5-a]pyrimidine-6-carboxamide

The relative and absolute stereochemistry of each example was not determined.

### Step 1: 2-(1-methyl-1H-pyrazol-3-yl)cyclopentan-1-ol

To a stirred solution of 3-iodo-1-methyl-1H-pyrazole (16.07 g, 77.27 mmol) in Et₂O (500 mL) was added dropwise n-Butyllithium (2.5 M, 30.9 mL) at −90° C. under nitrogen. The reaction mixture was stirred at −90° C. for 1 h under nitrogen. 6-oxabicyclo[3.1.0]hexane (5 g, 59.44 mmol) was added dropwise at −90° C. under nitrogen, and the mixture was stirred for 0.5 h at −90° C. Boron trifluoride diethyl etherate (10.97 g, 77.27 mmol) was added dropwise at −90° C., and the reaction mixture was stirred at −90° C. for 2 h under nitrogen. The reaction mixture was quenched with saturated aqueous ammonium chloride (500 mL). The resulting mixture was extracted with ethyl acetate (3×500 mL). The combined organic layers were dried over anhydrous sodium sulfate and concentrated under reduced pressure. The residue was applied onto a silica gel column and eluted with EtOAc/PE (1:1) to afford the title compound (6 g, 61% yield) as a yellow oil. LC-MS: m/z 167 [M+H]⁺.

### Step 2: 2-(1-methyl-1H-pyrazol-3-yl)cyclopent-2-en-1-one

In analogy to Method A1 step 2 but using 2-(1-methyl-1H-pyrazol-3-yl)cyclopentan-1-ol (15 g, 53.1 mmol), the title compound was obtained as a yellow oil (6 g, 36% yield). LC-MS: m/z 163 [M+H]⁺.

### Step 3: 2-(1-methyl-1H-pyrazol-3-yl)cyclopentan-1-one

To a stirred solution of 2-(1-methyl-1H-pyrazol-3-yl)cyclopent-2-en-1-one (6 g, 36.9 mmol) in MeOH (300 mL) was added Pd/C (3.0 g, 2.5 mmol, 10%). The mixture was degassed and then stirred at 25° C. for 5 h under hydrogen atmosphere. The mixture was filtered, and the filtrate was concentrated under reduced pressure to afford the crude title compound (6 g) as a yellow oil. LC-MS: m/z 165 [M+H]⁺.

### Step 4: 2-methyl-2-(1-methyl-1H-pyrazol-3-yl)cyclopentan-1-one

In analogy to Method A1 step 3 but using 2-(1-methyl-1H-pyrazol-3-yl)cyclopentan-1-one (5.9 g, 25.2 mmol) and after purification of the product by prep-HPLC, the title compound was obtained as a yellow oil (3.3 g, 62% yield). ¹H NMR (400 MHz, Chloroform-d) δ 7.28 (d, J=2.1 Hz, 1H), 6.18 (d, J=2.3 Hz, 1H), 3.87 (s, 3H), 2.55-2.66 (m, 1H), 2.29-2.49 (m, 2H), 1.86-2.08 (m, 3H), 1.40 (s, 3H). LC-MS: m/z 179 [M+H]⁺.

### Step 5: 5-((dimethylamino)methylene)-2-methyl-2-(1-methyl-1H-pyrazol-3-yl)cyclopentan-1-one

In analogy to Method A1 step 4 but using 2-methyl-2-(1-methyl-1H-pyrazol-3-yl)cyclopentan-1-one (3.4 g, 19.1 mmol) in 1-tert-butoxy-N,N,N′,N′-tetramethyl-methanediamine (6.6 g, 38.2 mmol) and stirring at 110° C. for 1 h, the crude title compound was obtained as a yellow oil (4.2 g). LC-MS: m/z 234 [M+H]⁺.

### Step 6: 2-Chloro-8-methyl-8-(1-methyl-1H-pyrazol-3-yl)-7,8-dihydro-6H-cyclopenta[e]pyrazolo[1,5-a]pyrimidine

In analogy to Method A1 step 5 but using 5-((dimethylamino)methylene)-2-methyl-2-(1-methyl-1H-pyrazol-3-yl)cyclopentan-1-one (5.9 g, 25.3 mmol) and 3-chloro-1H-pyrazol-5-amine (3.6 g, 30.4 mmol) and after purification of the product by prep-HPLC, the title compound was obtained as a yellow solid (2.5 g, 33% yield). LC-MS: m/z 288 [M+H]⁺.

### Step 7: 2-Chloro-8-methyl-8-(1-methyl-1H-pyrazol-3-yl)-7,8-dihydro-6H-cyclopenta[e]pyrazolo[1,5-a]pyrimidine-6-carbonitrile

In analogy to Method A1 step 6 but using 2-chloro-8-methyl-8-(1-methyl-1H-pyrazol-3-yl)-7,8-dihydro-6H-cyclopenta[e]pyrazolo[1,5-a]pyrimidine (1.5 g, 5.2 mmol) in chlorobenzene (250 mL), the title compound was obtained as a yellow solid (800 mg, 49% yield). LC-MS: m/z 313 [M+H]⁺.

### Step 8: 2-Chloro-8-methyl-8-(1-methyl-1H-pyrazol-3-yl)-7,8-dihydro-6H-cyclopenta[e]pyrazolo[1,5-a]pyrimidine-6-carboxylic acid

In analogy to Method A1 step 7 but using 2-chloro-8-methyl-8-(1-methyl-1H-pyrazol-3-yl)-7,8-dihydro-6H-cyclopenta[e]pyrazolo[1,5-a]pyrimidine-6-carbonitrile (1.1 g, 3.5 mmol) and after purification of the product by prep-HPLC, the title compound was obtained as a yellow solid (800 mg, 68% yield). LC-MS: m/z 332 [M+H]⁺.

### Step 9: trans-2-chloro-N-(5-chloro-6-(2H-1,2,3-triazol-2-yl)pyridin-3-yl)-8-methyl-8-(1-methyl-1H-pyrazol-3-yl)-7,8-dihydro-6H-cyclopenta[e]pyrazolo[1,5-a]pyrimidine-6-carboxamide and cis-2-chloro-N-(5-chloro-6-(2H-1,2,3-triazol-2-yl)pyridin-3-yl)-8-methyl-8-(1-methyl-1H-pyrazol-3-yl)-7,8-dihydro-6H-cyclopenta[e]pyrazolo[1,5-a]pyrimidine-6-carboxamide

In analogy to Method A1 step 8 but using 2-chloro-8-methyl-8-(1-methyl-1H-pyrazol-3-yl)-7,8-dihydro-6H-cyclopenta[e]pyrazolo[1,5-a]pyrimidine-6-carboxylic acid (100 mg, 301 mol) and 5-chloro-6-(triazol-2-yl)pyridin-3-amine (59 mg, 301 mol), racemic mixtures of the title compounds were obtained: trans isomer (30.5 mg, 83% yield) as a white solid, LC-MS: m/z 509 [M+H]⁺; cis-isomer (9.9 mg, 6% yield) as a white solid, LC-MS: m/z 509 [M+H]⁺.

### Step 10: Separation of Enantiomers to Obtain Example 76 and Example 77

29 mg of trans-2-chloro-N-(5-chloro-6-(2H-1,2,3-triazol-2-yl)pyridin-3-yl)-8-methyl-8-(1-methyl-1H-pyrazol-3-yl)-7,8-dihydro-6H-cyclopenta[e]pyrazolo[1,5-a]pyrimidine-6-carboxamide were submitted to chiral HPLC purification (Column: CHIRALPAK IF, 2*25 cm, 5 μm; Mobile Phase A: Hex: DCM=1:1(0.1% FA)-HPLC, Mobile Phase B: IPA-HPLC; Flow rate: 20 ML/MIN mL/min; Gradient: isocratic 10% Mobile Phase B; Wave Length: 254/220 nm nm; RT1 (min): 7.935; RT2 (min): 12.65; Sample Solvent: EtOH-HPLC; Injection Volume: 2.0 mL; Number Of Runs: 2). The second eluting isomer was concentrated and lyophilized to afford Example 76 (8.3 mg, 28% yield) as a white solid. The first eluting isomer was concentrated and lyophilized to afford Example 77 (8.4 mg, 28% yield) as a white solid.

Example 76: ¹H NMR (400 MHz, Chloroform-d) δ 8.77 (s, 1H), 8.70 (d, J=2 Hz, 1H), 8.57-8.60 (m, 2H), 7.94 (s, 2H), 7.36 (d, J=2 Hz, 1H), 6.73 (s, 1H), 6.68 (s, 1H), 4.69-4.73 (m, 1H), 3.95 (s, 3H), 3.50-3.59 (m, 1H), 2.52-2.58 (m, 1H), 2.14 (s, 3H). LC-MS: m/z 509 [M+H]⁺.

Example 77: ¹H NMR (400 MHz, Chloroform-d) δ 8.70 (d, J=2 Hz, 1H), 8.49-8.54 (m, 3H), 7.94 (s, 2H), 7.29 (d, J=2.4 Hz, 1H), 6.66 (s, 1H), 6.59 (d, J=2.4 Hz, 1H), 4.60-4.64 (m, 1H), 3.85 (s, 3H), 3.43-3.48 (m, 1H), 2.51-2.56 (m, 1H), 2.14 (s, 3H). LC-MS: m/z 509 [M+H]⁺.

### Step 11: c Example 78 and Example 79

9 mg of cis-2-chloro-N-(5-chloro-6-(2H-1,2,3-triazol-2-yl)pyridin-3-yl)-8-methyl-8-(1-methyl-1H-pyrazol-3-yl)-7,8-dihydro-6H-cyclopenta[e]pyrazolo[1,5-a]pyrimidine-6-carboxamide were submitted to chiral HPLC purification (Column: CHIRALPAK IF, 2*25 cm, 5 μm; Mobile Phase A: Hex: DCM=1:1(0.1% FA)-HPLC, Mobile Phase B: IPA-HPLC; Flow rate: 20 ML/MIN mL/min; Gradient: isocratic 7% Mobile Phase B; Wave Length: 254/220 nm nm; RT1 (min): 7.355; RT2 (min): 9.769; Sample Solvent: EtOH-HPLC; Injection Volume: 1.0 mL; Number Of Runs: 3). The first eluting isomer was concentrated and lyophilized to afford Example 78 (1.8 mg, 19% yield) as a white solid. The second eluting isomer was concentrated and lyophilized to afford Example 79 (2.1 mg, 23% yield) as a white solid.

Example 78: ¹H NMR (400 MHz, Chloroform-d) δ 10.70 (s, 1H), 8.55-8.62 (m, 3H), 7.92 (s, 2H), 7.40 (d, J=1.6 Hz, 1H), 6.75 (d, J=2.4 Hz, 1H), 6.68 (s, 1H), 4.51-4.54 (m, 1H), 3.86 (s, 3H), 3.26-3.30 (m, 1H), 2.83-2.89 (m, 1H), 2.11 (s, 3H). LC-MS: m/z 509 [M+H]⁺.

Example 79: ¹H NMR (400 MHz, Chloroform-d) δ 10.71 (s, 1H), 8.73 (s, 1H), 8.62-8.67 (m, 2H), 7.91 (s, 2H), 7.48 (s, 1H), 6.77 (d, J=2.4 Hz, 1H), 6.70 (s, 1H), 4.54-4.56 (m, 1H), 3.95 (s, 3H), 3.21-3.27 (m, 1H), 2.89-2.94 (m, 1H), 2.08 (s, 3H). LC-MS: m/z 509 [M+H]⁺.

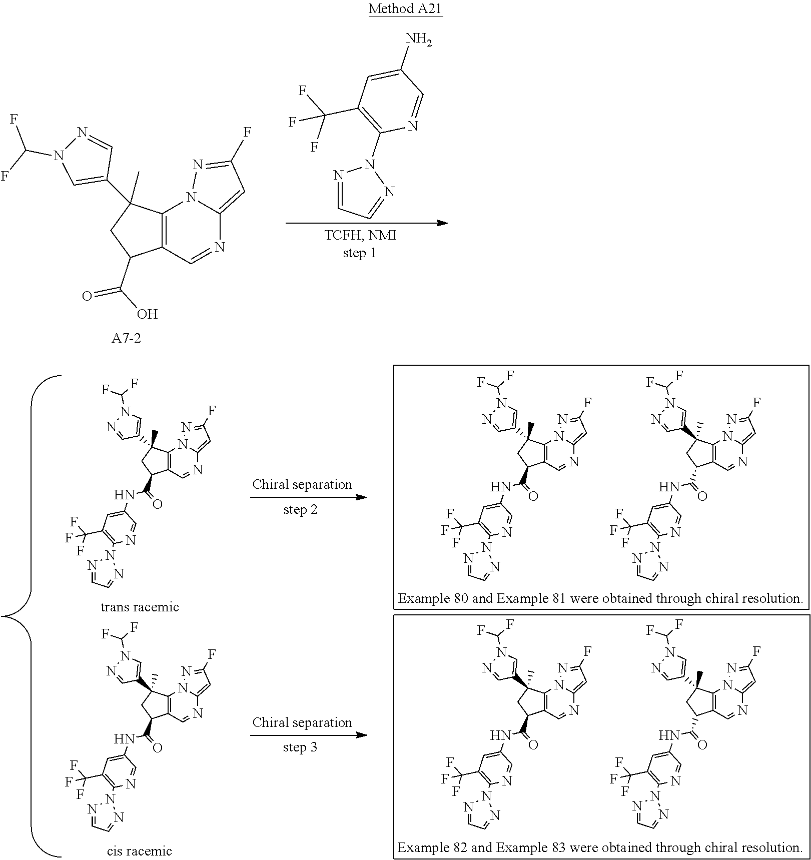

### Examples 80, 81: (6S,8R)—N-(6-(2H-1,2,3-triazol-2-yl)-5-(trifluoromethyl)pyridin-3-yl)-8-(1-(difluoromethyl)-1H-pyrazol-4-yl)-2-fluoro-8-methyl-7,8-dihydro-6H-cyclopenta[e]pyrazolo[1,5-a]pyrimidine-6-carboxamide; (6R,8S)—N-(6-(2H-1,2,3-triazol-2-yl)-5-(trifluoromethyl)pyridin-3-yl)-8-(1-(difluoromethyl)-1H-pyrazol-4-yl)-2-fluoro-8-methyl-7,8-dihydro-6H-cyclopenta[e]pyrazolo[1,5-a]pyrimidine-6-carboxamide

### Examples 82, 83: (6S,8S)—N-(6-(2H-1,2,3-triazol-2-yl)-5-(trifluoromethyl)pyridin-3-yl)-8-(1-(difluoromethyl)-1H-pyrazol-4-yl)-2-fluoro-8-methyl-7,8-dihydro-6H-cyclopenta[e]pyrazolo[1,5-a]pyrimidine-6-carboxamide; (6R,8R)—N-(6-(2H-1,2,3-triazol-2-yl)-5-(trifluoromethyl)pyridin-3-yl)-8-(1-(difluoromethyl)-1H-pyrazol-4-yl)-2-fluoro-8-methyl-7,8-dihydro-6H-cyclopenta[e]pyrazolo[1,5-a]pyrimidine-6-carboxamide

The relative and absolute stereochemistry of each example was not determined.

### Step 1: trans-N-(6-(2H-1,2,3-triazol-2-yl)-5-(trifluoromethyl)pyridin-3-yl)-8-(1-(difluoromethyl)-1H-pyrazol-4-yl)-2-fluoro-8-methyl-7,8-dihydro-6H-cyclopenta[e]pyrazolo[1,5-a]pyrimidine-6-carboxamide and cis-N-(6-(2H-1,2,3-triazol-2-yl)-5-(trifluoromethyl)pyridin-3-yl)-8-(1-(difluoromethyl)-1H-pyrazol-4-yl)-2-fluoro-8-methyl-7,8-dihydro-6H-cyclopenta[e]pyrazolo[1,5-a]pyrimidine-6-carboxamide

In analogy to Method A1 step 8 but using 8-(1-(difluoromethyl)-1H-pyrazol-4-yl)-2-fluoro-8-methyl-7,8-dihydro-6H-cyclopenta[c]pyrazolo[1,5-a]pyrimidine-6-carboxylic acid (A7-2, 230 mg, 655 μmol) and 6-(triazol-2-yl)-5-(trifluoromethyl)pyridin-3-amine (225 mg, 982 mol), racemic mixtures of the title compounds were obtained: trans isomer (180 mg, 48% yield) as a yellow solid, LC-MS: m/z 563 [M+H]⁺; cis-isomer (120 mg, 32% yield) as a yellow solid, LC-MS: m/z 563 [M+H]⁺.

### Step 2: Separation of Enantiomers to Obtain Example 80 and Example 81

180 mg of trans-N-(6-(2H-1,2,3-triazol-2-yl)-5-(trifluoromethyl)pyridin-3-yl)-8-(1-(difluoromethyl)-1H-pyrazol-4-yl)-2-fluoro-8-methyl-7,8-dihydro-6H-cyclopenta[e]pyrazolo[1,5-a]pyrimidine-6-carboxamide were submitted to chiral HPLC purification (Column: CHIRALPAK IF, 2*25 cm, 5 μm; Mobile Phase A: Hex: DCM=3:1(0.2% TFA)-HPLC, Mobile Phase B: EtOH-HPLC; Flow rate: 20 ML/MIN mL/min; Gradient: isocratic 10% Mobile Phase B; Wave Length: 254/220 nm nm; RT1 (min): 8.605; RT2 (min): 13.295; Sample Solvent: EtOH-HPLC; Injection Volume: 1.4 mL; Number Of Runs: 5). The second eluting isomer was concentrated and lyophilized to afford Example 80 (35.9 mg, 19% yield) as a light-yellow solid. The first eluting isomer was concentrated and lyophilized to afford Example 81 (18.4 mg, 10% yield) as a white solid.

Example 80: ¹H NMR (400 MHz, DMSO-d₆) δ 11.22 (s, 1H), 9.02 (d, J=2 Hz, 1H), 8.83 (d, J=2.4 Hz, 1H), 8.72 (s, 1H), 8.21 (s, 1H), 8.19 (s, 2H), 7.84 (s, 1H), 7.73 (t, J=59.2 Hz, 1H), 6.60 (d, J=4.8 Hz, 1H), 4.58 (t, J=8 Hz, 1H), 2.92-2.95 (m, 1H), 2.58-2.63 (m, 1H), 2.00 (s, 3H). LC-MS: m/z 563 [M+H]⁺.

Example 81: ¹H NMR (400 MHz, DMSO-d₆) δ 11.22 (s, 1H), 9.02 (d, J=2.4 Hz, 1H), 8.83 (d, J=2 Hz, 1H), 8.72 (s, 1H), 8.21 (s, 1H), 8.19 (s, 2H), 7.84 (s, 1H), 7.73 (t, J=59.2 Hz, 1H), 6.60 (d, J=4.8 Hz, 111), 4.58 (t, J=8 Hz, 111), 2.92-2.95 (m, 1H), 2.58-2.63 (m, 1H), 2.00 (s, 3H). LC-MS: m/z 563 [M+H]⁺.

### Step 3: Separation of Enantiomers to Obtain Example 82 and Example 83

120 mg of cis-N-(6-(2H-1,2,3-triazol-2-yl)-5-(trifluoromethyl)pyridin-3-yl)-8-(1-(difluoromethyl)-1H-pyrazol-4-yl)-2-fluoro-8-methyl-7,8-dihydro-6H-cyclopenta[e]pyrazolo[1,5-a]pyrimidine-6-carboxamide were submitted to chiral HPLC purification (Column: CHIRALPAK IG 2*25 cm, 5 μm; Mobile Phase A: Hex: DCM=3:1(0.1% FA)-HPLC, Mobile Phase B: EtOH-HPLC; Flow rate: 20 ML/MIN mL/min; Gradient: isocratic 10% Mobile Phase B; Wave Length: 254/220 NM nm; RT1 (min): 9.392; RT2 (min): 10.063; Sample Solvent: EtOH-HPLC; Injection Volume: 1.4 mL; Number Of Runs: 5). The second eluting isomer was concentrated and lyophilized to afford Example 82 (21.4 mg, 17% yield) as a white solid. The first eluting isomer was concentrated and lyophilized to afford Example 83 (12.9 mg, 10% yield) as a white solid.

Example 82: ¹H NMR (400 MHz, DMSO-d₆) δ 11.21 (s, 1H), 9.01 (d, J=2.4 Hz, 1H), 8.76-8.79 (m, 2H), 8.18-8.19 (m, 3H), 7.77 (s, 1H), 7.72 (t, J=59.2 Hz, 1H), 6.58 (d, J=4.8 Hz, 1H), 4.59-4.61 (m, 1H), 2.84-2.90 (m, 1H), 2.74-2.80 (m, 1H), 1.91 (s, 3H). LC-MS: m/z 563 [M+H]⁺.

Example 83: ¹H NMR (400 MHz, DMSO-d₆) δ:11.20 (s, 1H), 9.01 (d, J=2 Hz, 1H), 8.76-8.79 (m, 2H), 8.18-8.19 (m, 3H), 7.77 (s, 1H), 7.72 (t, J=59.2 Hz, 1H), 6.58 (d, J=5.2 Hz, 1H), 4.56-4.60 (m, 1H), 2.84-2.90 (m, 1H), 2.74-2.80 (m, 1H), 1.91 (s, 3H). LC-MS: m/z 563 [M+H]⁺.

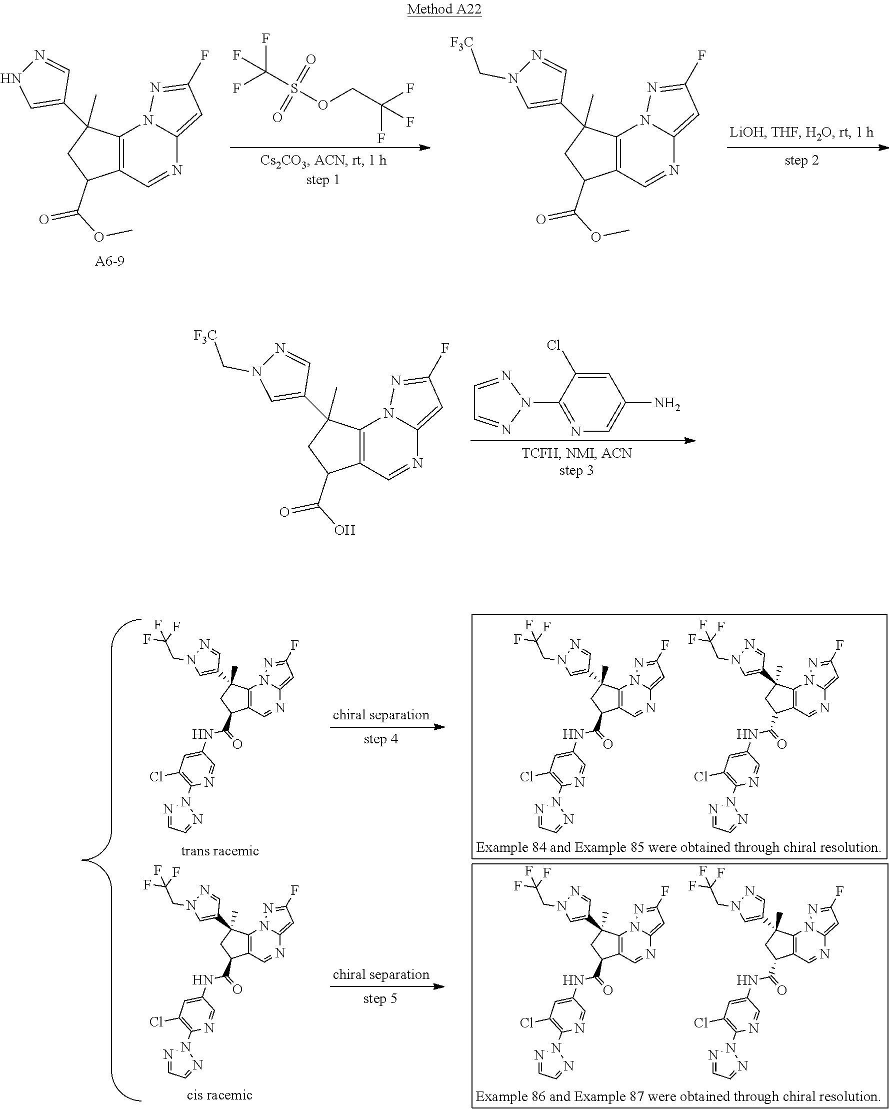

### Examples 84, 85: (6S,8R)—N-(5-chloro-6-(2H-1,2,3-triazol-2-yl)pyridin-3-yl)-2-fluoro-8-methyl-8-(1-(2,2,2-trifluoroethyl)-1H-pyrazol-4-yl)-7,8-dihydro-6H-cyclopenta[e]pyrazolo[1,5-a]pyrimidine-6-carboxamide; (6R,8S)—N-(5-chloro-6-(2H-1,2,3-triazol-2-yl)pyridin-3-yl)-2-fluoro-8-methyl-8-(1-(2,2,2-trifluoroethyl)-1H-pyrazol-4-yl)-7,8-dihydro-6H-cyclopenta[e]pyrazolo[1,5-a]pyrimidine-6-carboxamide

### Examples 86, 87: (6S,8S)—N-(5-chloro-6-(2H-1,2,3-triazol-2-yl)pyridin-3-yl)-2-fluoro-8-methyl-8-(1-(2,2,2-trifluoroethyl)-1H-pyrazol-4-yl)-7,8-dihydro-6H-cyclopenta[e]pyrazolo[1,5-a]pyrimidine-6-carboxamide; (6R,8R)—N-(5-chloro-6-(2H-1,2,3-triazol-2-yl)pyridin-3-yl)-2-fluoro-8-methyl-8-(1-(2,2,2-trifluoroethyl)-1H-pyrazol-4-yl)-7,8-dihydro-6H-cyclopenta[e]pyrazolo[1,5-a]pyrimidine-6-carboxamide

The relative and absolute stereochemistry of each example was not determined.

### Step 1: Methyl 2-fluoro-8-methyl-8-(1-(2,2,2-trifluoroethyl)-1H-pyrazol-4-yl)-7,8-dihydro-6H-cyclopenta[e]pyrazolo[1,5-a]pyrimidine-6-carboxylate

To a stirred solution of methyl 2-fluoro-8-methyl-8-(1H-pyrazol-4-yl)-7,8-dihydro-6H-cyclopenta[e]pyrazolo[1,5-a]pyrimidine-6-carboxylate (A6-9, 225 mg, 711 μmol) in ACN (10 mL) were added Cesium carbonate (1 g, 3.0 mmol) and 2,2,2-trifluoroethyl trifluoromethanesulfonate (330 mg, 1.4 mmol). The reaction mixture was stirred at 25° C. for 1 h. The reaction was quenched with water (20 mL), and the resulting mixture was extracted with ethyl acetate (3×20 mL). The combined organic layers were dried over anhydrous sodium sulfate and concentrated under reduced pressure. The residue was purified by column chromatography on silica gel using 90% DCM and 10% MeOH as eluent to afford the title compound (170 mg, 54% yield) as a yellow oil. LC-MS: m/z 398 [M+H]⁺.

### Step 2: 2-Fluoro-8-methyl-8-(1-(2,2,2-trifluoroethyl)-1H-pyrazol-4-yl)-7,8-dihydro-6H-cyclopenta[e]pyrazolo[1,5-a]pyrimidine-6-carboxylic acid

To a stirred solution of methyl 2-fluoro-8-methyl-8-(1-(2,2,2-trifluoroethyl)-1H-pyrazol-4-yl)-7,8-dihydro-6H-cyclopenta[e]pyrazolo[1,5-a]pyrimidine-6-carboxylate (170 mg, 428 μmol) in THF (1.5 mL) was added LiOH (103 mg, 4.3 mmol) in water (0.5 mL). The reaction mixture was stirred at 25° C. for 1 h. The pH was adjusted to ˜6 with 1M HCl, and the resulting mixture was extracted with ethyl acetate (3×10 mL). The combined organic layers were dried over anhydrous sodium sulfate and concentrated under reduced pressure. The residue was purified by column chromatography on silica gel using 90% DCM and 10% MeOH as eluent to afford the title compound (140 mg, 68% yield) as a yellow oil. LC-MS: m/z 384[M+H]⁺.

### Step 3: trans-N-(5-chloro-6-(2H-1,2,3-triazol-2-yl)pyridin-3-yl)-2-fluoro-8-methyl-8-(1-(2,2,2-trifluoroethyl)-1H-pyrazol-4-yl)-7,8-dihydro-6H-cyclopenta[e]pyrazolo[1,5-a]pyrimidine-6-carboxamide and cis-N-(5-chloro-6-(2H-1,2,3-triazol-2-yl)pyridin-3-yl)-2-fluoro-8-methyl-8-(1-(2,2,2-trifluoroethyl)-1H-pyrazol-4-yl)-7,8-dihydro-6H-cyclopenta[e]pyrazolo[1,5-a]pyrimidine-6-carboxamide

In analogy to Method A1 step 8 but using 2-fluoro-8-methyl-8-(1-(2,2,2-trifluoroethyl)-1H-pyrazol-4-yl)-7,8-dihydro-6H-cyclopenta[e]pyrazolo[1,5-a]pyrimidine-6-carboxylic acid (130 mg, 339 mol) and 5-chloro-6-(triazol-2-yl)pyridin-3-amine (80 mg, 407 mol), racemic mixtures of the title compounds were obtained: trans isomer (57 mg, 29% yield) as a white solid, LC-MS: m/z 561[M+H]⁺; cis-isomer (37 mg, 19% yield) as a white solid, LC-MS: m/z 561[M+H]⁺.

### Step 4: Separation of Enantiomers to Obtain Example 84 and Example 85

50 mg of trans-N-(5-chloro-6-(2H-1,2,3-triazol-2-yl)pyridin-3-yl)-2-fluoro-8-methyl-8-(1-(2,2,2-trifluoroethyl)-1H-pyrazol-4-yl)-7,8-dihydro-6H-cyclopenta[e]pyrazolo[1,5-a]pyrimidine-6-carboxamide were submitted to chiral HPLC purification (Column: CHIRAL ART Amylose-SA 2×25 cm, 5 μm; Mobile Phase A: Hex: DCM=3:1(0.1% FA)-HPLC, Mobile Phase B: EtOH-HPLC; Flow rate: 20 ML/MIN mL/min; Gradient: isocratic 10% Mobile Phase B; Wave Length: 254/220 NM nm; RT1 (min): 10.463; RT2 (min): 15.53; Sample Solvent: EtOH-HPLC; Injection Volume: 3 mL; Number Of Runs: 5). The second eluting isomer was concentrated and lyophilized to afford Example 84 (8.1 mg, 16% yield) as a white solid. The first eluting isomer was concentrated and lyophilized to afford Example 85 (8.6 mg, 17% yield) as a white solid.

Example 84: ¹H NMR (400 MHz, Chloroform-d) δ 9.45 (s, 1H), 8.94 (s, 11H), 8.67-8.70 (m, 2H), 7.91 (s, 2H), 7.68-7.72 (m, 2H), 6.40 (d, J=4.4 Hz, 1H), 4.64-4.70 (m, 3H), 3.08-3.12 (m, 1H), 2.83-2.88 (m, 1H), 2.14 (s, 3H). LC-MS: m/z 561[M+H]⁺.

Example 85: ¹H NMR (400 MHz, Chloroform-d) δ 9.46 (s, 1H), 8.94 (s, 11H), 8.67-8.74 (m, 214), 7.92 (s, 2H), 7.68-7.72 (m, 2H), 6.42 (d, J=4.4 Hz, 1H), 4.66-4.71 (m, 3H), 3.08-3.11 (m, 1H), 2.83-2.90 (m, 1H), 2.15 (s, 3H). LC-MS: m/z 561[M+H]⁺.

### Step 5: Separation of Enantiomers to Obtain Example 86 and Example 87

30 mg of cis-N-(5-chloro-6-(2H-1,2,3-triazol-2-yl)pyridin-3-yl)-2-fluoro-8-methyl-8-(1-(2,2,2-trifluoroethyl)-1H-pyrazol-4-yl)-7,8-dihydro-6H-cyclopenta[e]pyrazolo[1,5-a]pyrimidine-6-carboxamide were submitted to chiral HPLC purification (Column: CHIRAL ART Amylose-SA 2×25 cm, 5 μm; Mobile Phase A: Hex: DCM=3:1(0.1% FA)-HPLC, Mobile Phase B: EtOH-HPLC; Flow rate: 20 ML/MIN mL/min; Gradient: isocratic 10% Mobile Phase B; Wave Length: 254/220 NM nm; RT1 (min): 7.245; RT2 (min): 8.763; Sample Solvent: EtOH-HPLC; Injection Volume: 2.5 mL; Number Of Runs: 5). The second eluting isomer was concentrated and lyophilized to afford Example 86 (4.8 mg, 15% yield) as a white solid. The first eluting isomer was concentrated and lyophilized to afford Example 87 (4.2 mg, 13% yield) as a white solid.

Example 86: ¹H NMR (400 MHz, Chloroform-d) δ 9.06-9.19 (m, 211), 8.60-8.68 (m, 2H), 7.90 (s, 211), 7.78-7.82 (m, 211), 6.43 (d, J=5.2 Hz, 1H), 4.63-4.70 (m, 3H), 3.14-3.21 (m, 1H), 2.82-2.92 (m, 1H), 2.02 (s, 311). LC-MS: m/z 561[M+H]⁺.

Example 87: ¹H NMR (400 MHz, Chloroform-d) δ 8.90 (s, 1H), 8.63 (s, 1H), 8.56 (s, 1H), 7.91 (s, 2H), 7.73-7.75 (m, 2H), 6.38 (d, J=5.2 Hz, 111), 4.63-4.69 (m, 211), 4.50-4.54 (m, 111), 3.10-3.14 (m, 1H), 2.85-2.90 (m, 1H), 2.01 (s, 3H). LC-MS: m/z 561[M+H]⁺.

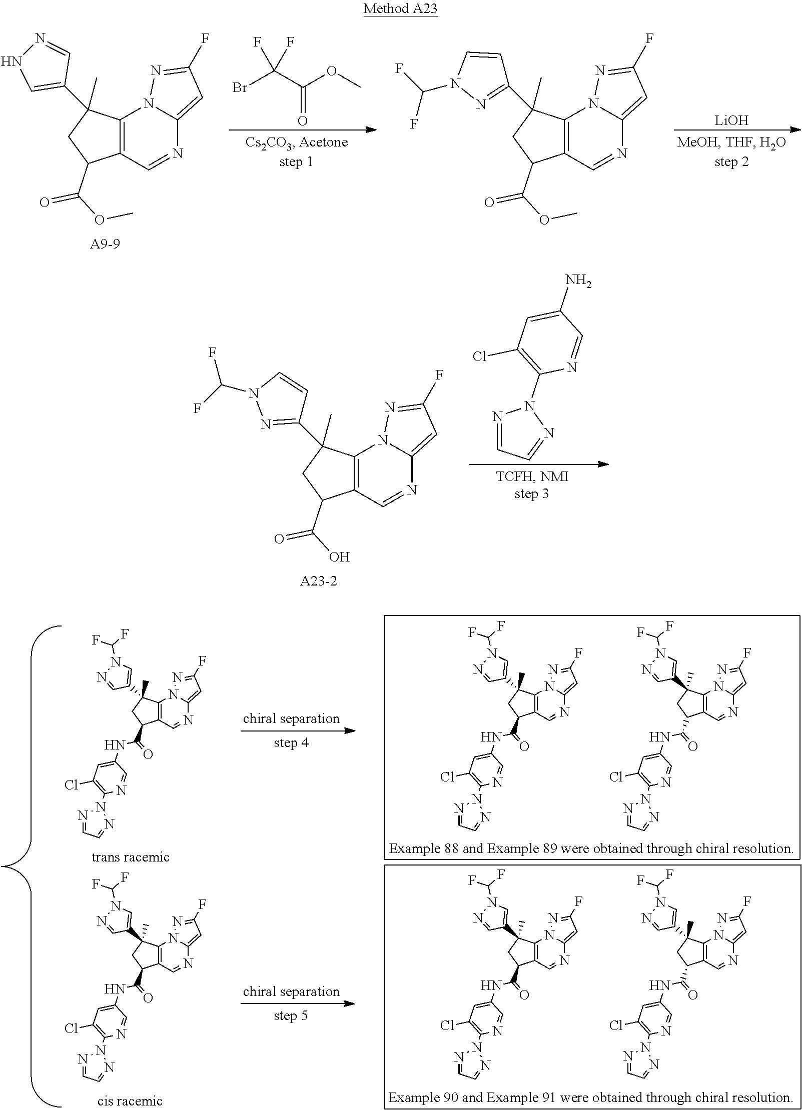

### Examples 88, 89: (6S,8S)—N-(5-chloro-6-(2H-1,2,3-triazol-2-yl)pyridin-3-yl)-8-(1-(difluoromethyl)-1H-pyrazol-3-yl)-2-fluoro-8-methyl-7,8-dihydro-6H-cyclopenta[e]pyrazolo[1,5-a]pyrimidine-6-carboxamide; (6R,8R)—N-(5-chloro-6- (2H-1,2,3-triazol-2-yl)pyridin-3-yl)-8-(1-(difluoromethyl)-1H-pyrazol-3-yl)-2-fluoro-8-methyl-7,8-dihydro-6H-cyclopenta[e]pyrazolo[1,5-a]pyrimidine-6-carboxamide

### Examples 90, 91: (6S,8R)—N-(5-chloro-6-(2H-1,2,3-triazol-2-yl)pyridin-3-yl)-8-(1-(difluoromethyl)-1H-pyrazol-3-yl)-2-fluoro-8-methyl-7,8-dihydro-6H-cyclopenta[e]pyrazolo[1,5-a]pyrimidine-6-carboxamide; (6R,8S)—N-(5-chloro-6-(2H-1,2,3-triazol-2-yl)pyridin-3-yl)-8-(1-(difluoromethyl)-1H-pyrazol-3-yl)-2-fluoro-8-methyl-7,8-dihydro-6H-cyclopenta[e]pyrazolo[1,5-a]pyrimidine-6-carboxamide

The relative and absolute stereochemistry of each example was not determined.

### Step 1: Methyl 8-(1-(difluoromethyl)-1H-pyrazol-3-yl)-2-fluoro-8-methyl-7,8-dihydro-6H-cyclopenta[e]pyrazolo[1,5-a]pyrimidine-6-carboxylate

To a stirred solution of methyl 2-fluoro-8-methyl-8-(1H-pyrazol-3-yl)-7,8-dihydro-6H-cyclopenta[e]pyrazolo[1,5-a]pyrimidine-6-carboxylate (A9-9, 500 mg, 1.6 mmol) in acetone (15 mL) were added cesium carbonate (1.6 g, 4.7 mmol) and methyl 2-bromo-2,2-difluoroacetate (1.5 g, 7.9 mmol). The mixture was stirred at 40° C. for 16 h. The reaction was quenched with water (40 mL). The resulting mixture was extracted with ethyl acetate (3×40 mL). The combined organic layers were dried over anhydrous sodium sulfate and concentrated under reduced pressure. The residue was applied on a silica gel column and eluted with EtOAc/PE (1:1) to afford the title compound (90 mg, 13% yield) as a yellow oil. LC-MS: m/z 366 [M+H]⁺.

### Step 2: 8-(1-(difluoromethyl)-1H-pyrazol-3-yl)-2-fluoro-8-methyl-7,8-dihydro-6H-cyclopenta[e]pyrazolo[1,5-a]pyrimidine-6-carboxylic acid (A23-2)

In analogy to Method A7 step 2 but using methyl 8-(1-(difluoromethyl)-1H-pyrazol-3-yl)-2-fluoro-8-methyl-7,8-dihydro-6H-cyclopenta[e]pyrazolo[1,5-a]pyrimidine-6-carboxylate (90 mg, 246 mol), the crude title compound was obtained as a yellow oil (A23-2, 50 mg, 46% yield). LC-MS: m/z 352 [M+H]⁺.

### Step 3: trans-N-(5-chloro-6-(2H-1,2,3-triazol-2-yl)pyridin-3-yl)-8-(1-(difluoromethyl)-1H-pyrazol-3-yl)-2-fluoro-8-methyl-7,8-dihydro-6H-cyclopenta[e]pyrazolo[1,5-a]pyrimidine-6-carboxamide and cis-N-(5-chloro-6-(2H-1,2,3-triazol-2-yl)pyridin-3-yl)-8-(1-(difluoromethyl)-1H-pyrazol-3-yl)-2-fluoro-8-methyl-7,8-dihydro-6H-cyclopenta[e]pyrazolo[1,5-a]pyrimidine-6-carboxamide

In analogy to Method A1 step 8 but using 8-(1-(difluoromethyl)-1H-pyrazol-3-yl)-2-fluoro-8-methyl-7,8-dihydro-6H-cyclopenta[e]pyrazolo[1,5-a]pyrimidine-6-carboxylic acid (150 mg, 427 μmol) and 5-chloro-6-(triazol-2-yl)pyridin-3-amine (100 mg, 512 mol), racemic mixtures of the title compounds were obtained: trans isomer (120 mg, 53% yield) as a white solid, LC-MS: m/z 529 [M+H]⁺; cis-isomer (40 mg, 18% yield) as a white solid, LC-MS: m/z 529 [M+H]⁺.

### Step 4: Separation of Enantiomers to Obtain Example 88 and Example 89

120 mg of trans-N-(5-chloro-6-(2H-1,2,3-triazol-2-yl)pyridin-3-yl)-8-(1-(difluoromethyl)-1H-pyrazol-3-yl)-2-fluoro-8-methyl-7,8-dihydro-6H-cyclopenta[e]pyrazolo[1,5-a]pyrimidine-6-carboxamide were submitted to chiral HPLC purification (Column: CHIRALPAK IF, 2*25 cm, 5 μm; Mobile Phase A: Hex: DCM=3:1(0.1% FA)-HPLC, Mobile Phase B: EtOH-HPLC; Flow rate: 20 ML/MIN mL/min; Gradient: isocratic 30% Mobile Phase B; Wave Length: 254/220 nm nm; RT1 (min): 5.826; RT2 (min): 9.373; Sample Solvent: EtOH-HPLC; Injection Volume: 1.2 mL; Number Of Runs: 5.). The second eluting isomer was concentrated and lyophilized to afford Example 88 (21.4 mg, 35% yield) as a white solid. The first eluting isomer was concentrated and lyophilized to afford Example 89 (20.9 mg, 34% yield) as a white solid.

Example 88: ¹H NMR (400 MHz, Chloroform-d) δ 8.74 (s, 1H), 8.66 (s, 1H), 8.57 (s, 1H), 8.21 (s, 1H), 7.96 (s, 2H), 7.76 (d, J=2.4 Hz, 1H), 7.06 (t, J=60.8 Hz, 1H), 6.79 (d, J=2.8 Hz, 1H), 6.33 (d, J=5.2 Hz, 1H), 4.58 (t, J=8.2 Hz, 1H), 3.41-3.46 (m, 1H), 2.67-2.61 (m, 1H), 2.17 (s, 3H). LC-MS: m/z 529 [M+H]⁺.

Example 89: ¹H NMR (400 MHz, Chloroform-d) δ 8.74 (d, J=1.6 Hz, 2H), 8.61 (s, 1H), 8.39 (s, 1H), 7.96 (s, 2H), 7.76 (d, J=2.8 Hz, 1H), 7.06 (t, J=60.8 Hz, 1H), 6.78 (d, J=2.4 Hz, 1H), 6.35 (d, J=3.9 Hz, 1H), 4.61 (d, J=8 Hz, 1H), 3.40-3.45 (m, 1H), 2.66-2.71 (m, 1H), 2.18 (s, 3H). LC-MS: m/z 529 [M+H]⁺.

### Step 5: Separation of Enantiomers to Obtain Example 90 and Example 91

40 mg of cis-N-(5-chloro-6-(2H-1,2,3-triazol-2-yl)pyridin-3-yl)-8-(1-(difluoromethyl)-1H-pyrazol-3-yl)-2-fluoro-8-methyl-7,8-dihydro-6H-cyclopenta[e]pyrazolo[1,5-a]pyrimidine-6-carboxamide were submitted to chiral HPLC purification (Column: CHIRALPAK IG, 2*25 cm, 5 μm; Mobile Phase A: Hex: DCM=3:1(0.1% FA)-HPLC, Mobile Phase B: IPA-HPLC; Flow rate: 20 ML/MIN mL/min; Gradient: isocratic 20% Mobile Phase B; Wave Length: 254/220 nm nm; RT1 (min): 8.144; RT2 (min): 13.369; Sample Solvent: EtOH-HPLC; Injection Volume: 1.5 mL; Number Of Runs: 3.). The second eluting isomer was concentrated and lyophilized to afford Example 90 (14.2 mg, 71% yield) as a white solid. The first eluting isomer was concentrated and lyophilized to afford Example 91 (8.9 mg, 44% yield) as a white solid.

Example 90: ¹H NMR (400 MHz, Chloroform-d) δ 9.43 (s, 1H), 8.66 (s, 1H), 8.50-8.52 (m, 1H), 8.47 (s, 1H), 7.94 (s, 2H), 7.83 (d, J=2.4 Hz, 1H), 6.84-7.15 (m, 2H), 6.32 (d, J=5.2 Hz, 1H), 4.54-4.58 (m, 1H), 3.36-3.41 (m, 1H), 2.86-2.93 (m, 1H), 2.15 (s, 3H). LC-MS: m/z 529 [M+H]⁺.

Example 91: ¹H NMR (400 MHz, Chloroform-d) δ 9.41 (s, 1H), 8.75 (s, 1H), 8.52-8.67 (m, 2H), 7.95 (s, 2H), 7.83 (d, J=2.4 Hz, 1H), 6.84-7.15 (m, 2H), 6.32 (d, J=4.4 Hz, 1H), 4.54-4.57 (m, 1H), 3.36-3.41 (m, 1H), 2.86-2.92 (m, 1H), 2.16 (s, 3H). LC-MS: m/z 529 [M+H]⁺.

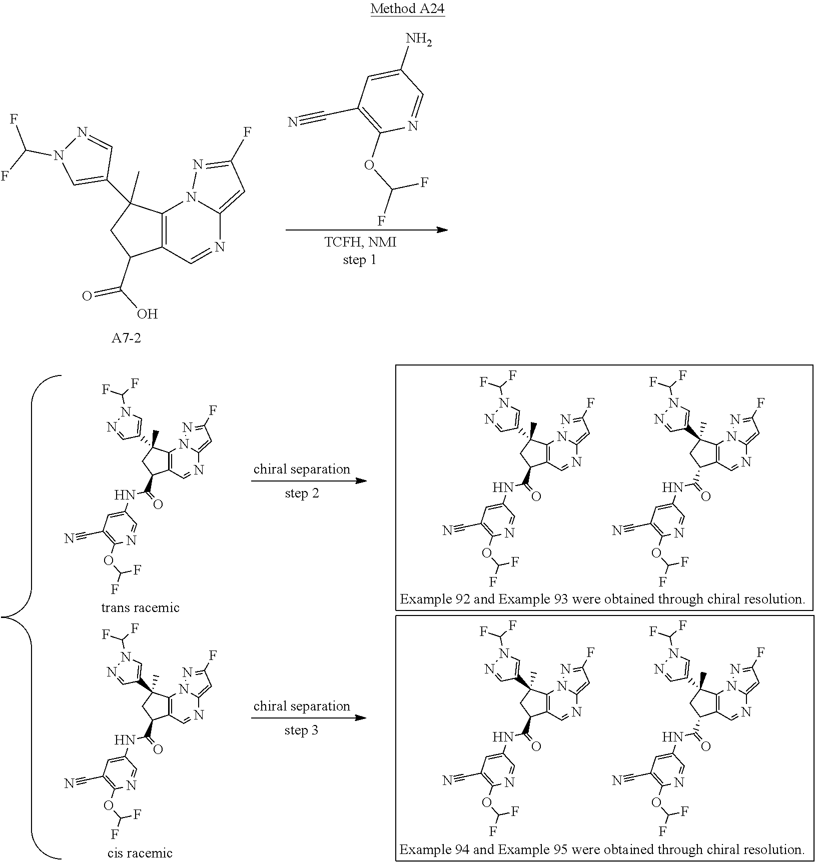

### Examples 92, 93: (6S,8R)—N-(5-cyano-6-(difluoromethoxy)pyridin-3-yl)-8-(1-(difluoromethyl)-1H-pyrazol-4-yl)-2-fluoro-8-methyl-7,8-dihydro-6H-cyclopenta[e]pyrazolo[1,5-a]pyrimidine-6-carboxamide; (6R,8S)—N-(5-cyano-6-(difluoromethoxy)pyridin-3-yl)-8-(1-(difluoromethyl)-1H-pyrazol-4-yl)-2-fluoro-8-methyl-7,8-dihydro-6H-cyclopenta[e]pyrazolo[1,5-a]pyrimidine-6-carboxamide

### Examples 94, 95: (6S,8S)—N-(5-cyano-6-(difluoromethoxy)pyridin-3-yl)-8-(1-(difluoromethyl)-1H-pyrazol-4-yl)-2-fluoro-8-methyl-7,8-dihydro-6H-cyclopenta[e]pyrazolo[1,5-a]pyrimidine-6-carboxamide; (6R,8R)—N-(5-cyano-6-(difluoromethoxy)pyridin-3-yl)-8-(1-(difluoromethyl)-1H-pyrazol-4-yl)-2-fluoro-8-methyl-7,8-dihydro-6H-cyclopenta[e]pyrazolo[1,5-a]pyrimidine-6-carboxamide

The relative and absolute stereochemistry of each example was not determined.

### Step 1: trans-N-(5-cyano-6-(difluoromethoxy)pyridin-3-yl)-8-(1-(difluoromethyl)-1H-pyrazol-4-yl)-2-fluoro-8-methyl-7,8-dihydro-6H-cyclopenta[e]pyrazolo[1,5-a]pyrimidine-6-carboxamide and cis-N-(5-cyano-6-(difluoromethoxy)pyridin-3-yl)-8-(1-(difluoromethyl)-1H-pyrazol-4-yl)-2-fluoro-8-methyl-7,8-dihydro-6H-cyclopenta[e]pyrazolo[1,5-a]pyrimidine-6-carboxamide

In analogy to Method A1 step 8 but using 8-(1-(difluoromethyl)-1H-pyrazol-4-yl)-2-fluoro-8-methyl-7,8-dihydro-6H-cyclopenta[e]pyrazolo[1,5-a]pyrimidine-6-carboxylic acid (A7-2, 240 mg, 683 μmol) and 5-amino-2-(difluoromethoxy)pyridine-3-carbonitrile (190 mg, 1.0 mmol), racemic mixtures of the title compounds were obtained: trans isomer (60 mg, 16% yield) as a white solid, LC-MS: m/z 519[M+H]⁺; cis-isomer (40 mg, 11% yield) as a white solid, LC-MS: m/z 519[M+H]⁺.

### Step 2: Separation of Enantiomers to Obtain Example 92 and Example 93

60 mg of trans-N-(5-cyano-6-(difluoromethoxy)pyridin-3-yl)-8-(1-(difluoromethyl)-1H-pyrazol-4-yl)-2-fluoro-8-methyl-7,8-dihydro-6H-cyclopenta[e]pyrazolo[1,5-a]pyrimidine-6-carboxamide were submitted to chiral HPLC purification (Column: CHIRALPAK IF, 2×25 cm, 5 μm; Mobile Phase A: Hex: DCM=3:1(0.1% FA)-HPLC, Mobile Phase B: EtOH-HPLC; Flow rate: 20 ML/MIN mL/min; Gradient: isocratic 30% Mobile Phase B; Wave Length: 254/220 nm nm; RT1 (min): 4.269; RT2 (min): 7.473; Sample Solvent: EtOH-HPLC; Injection Volume: 1.5 mL; Number Of Runs: 3). The second eluting isomer was concentrated and lyophilized to afford Example 92 (15.0 mg, 24% yield) as a white solid. The first eluting isomer was concentrated and lyophilized to afford Example 93 (19.4 mg, 32% yield) as a white solid.

Example 92: ¹H NMR (400 MHz, DMSO-d₆) δ 10.88 (s, 1H), 8.63-8.68 (m, 3H), 8.20 (s, 1H), 7.57-7.93 (m, 3H), 6.59 (d, J=4.8 Hz, 1H), 4.48-4.52 (m, 1H), 2.87-2.90 (m, 1H), 2.57-2.60 (m, 1H), 1.98 (s, 3H). LC-MS: m/z 519[M+H]⁺.

Example 93: ¹H NMR (400 MHz, DMSO-d₆) δ 10.89 (s, 1H), 8.63-8.68 (m, 3H), 8.20 (s, 1H), 7.57-7.93 (m, 3H), 6.59 (d, J=4.8 Hz, 1H), 4.48-4.52 (m, 1H), 2.85-2.92 (m, 1H), 2.54-2.60 (m, 1H), 1.98 (s, 3H). LC-MS: m/z 519[M+H]⁺.

### Step 3: Separation of Enantiomers to Obtain Example 94 and Example 95

40 mg of cis-N-(5-cyano-6-(difluoromethoxy)pyridin-3-yl)-8-(1-(difluoromethyl)-1H-pyrazol-4-yl)-2-fluoro-8-methyl-7,8-dihydro-6H-cyclopenta[e]pyrazolo[1,5-a]pyrimidine-6-carboxamide were submitted to chiral HPLC purification (Column: CHIRALPAK IF, 2×25 cm, 5 m; Mobile Phase A: Hex: DCM=3:1(0.1% FA)-HPLC, Mobile Phase B: EtOH-HPLC; Flow rate: 20 ML/MIN mL/min; Gradient: isocratic 30% Mobile Phase B; Wave Length: 254/220 nm nm; RT1 (min): 4.168; RT2 (min): 5.831; Sample Solvent: EtOH-HPLC; Injection Volume: 0.9 mL; Number Of Runs: 5). The second eluting isomer was concentrated and lyophilized to afford Example 94 (10.1 mg, 25% yield) as a white solid. The first eluting isomer was concentrated and lyophilized to afford Example 95 (12.1 mg, 26% yield) as a white solid.

Example 94: ¹H NMR (400 MHz, DMSO-d₆) δ 10.88 (s, 1H), 8.71 (s, 1H), 8.66 (d, J=2.4 Hz, 1H), 8.59 (d, J=2.8 Hz, 1H), 8.18 (s, 1H), 7.56-7.92 (m, 3H), 6.57 (d, J=5.2 Hz, 1H), 4.49-4.52 (m, 1H), 2.80-2.85 (m, 1H), 2.66-2.72 (m, 1H), 1.89 (s, 3H). LC-MS: m/z 519[M+H]⁺.

Example 95: ¹H NMR (400 MHz, DMSO-d₆) δ 10.88 (s, 1H), 8.71 (s, 1H), 8.66 (d, J=2.8 Hz, 1H), 8.59 (d, J=2.4 Hz, 1H), 8.18 (s, 1H), 7.56-7.92 (m, 3H), 6.57 (d, J=5.2 Hz, 1H), 4.49-4.51 (m, 1H), 2.80-2.85 (m, 1H), 2.68-2.72 (m, 1H), 1.89 (s, 3H). LC-MS: m/z 519[M+H]⁺.

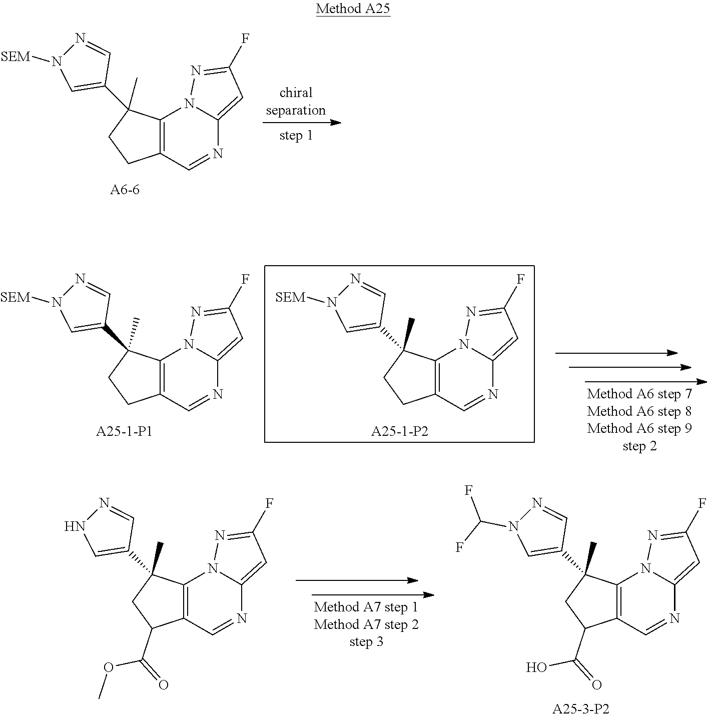 [SMILES: CC1(c2cnn(COCC[Si](C)(C)C)c2)CCc2cnc3cc(F)nn3c21 ; C[C@@]1(c2cnn(COCC[Si](C)(C)C)c2)CCc2cnc3cc(F)nn3c21 ; C[C@]1(c2cnn(COCC[Si](C)(C)C)c2)CCc2cnc3cc(F)nn3c21 ; COC(=O)C1C[C@](C)(c2cn[nH]c2)c2c1cnc1cc(F)nn21 ; C[C@]1(c2cnn(C(F)F)c2)CC(C(=O)O)c2cnc3cc(F)nn3c21]

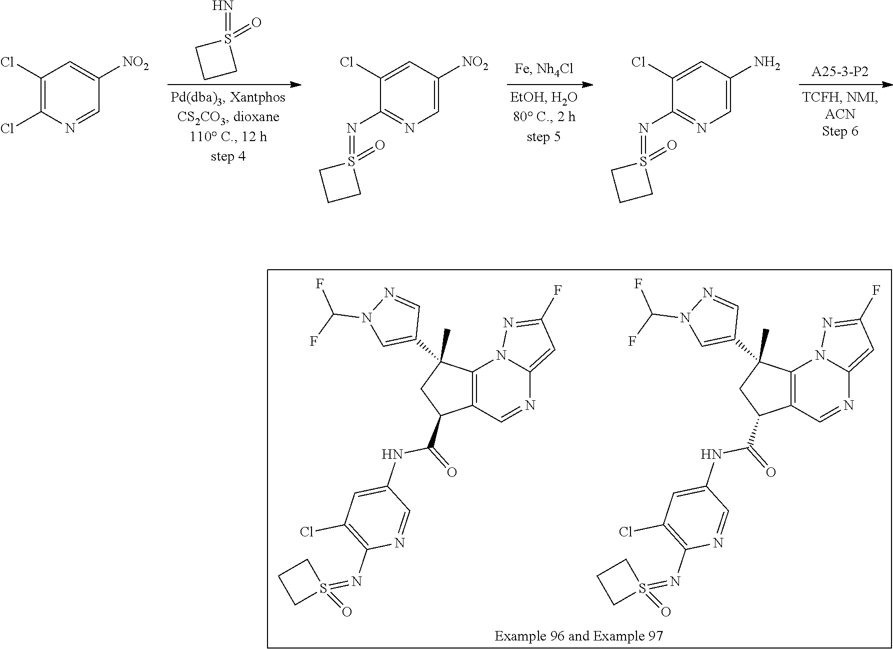 [SMILES: O=[N+]([O-])c1cnc(Cl)c(Cl)c1 ; N=S1(=O)CCC1 ; O=[N+]([O-])c1cnc(N=S2(=O)CCC2)c(Cl)c1 ; Nc1cnc(N=S2(=O)CCC2)c(Cl)c1 ; C[C@]1(c2cnn(C(F)F)c2)C[C@H](C(=O)Nc2cnc(N=S3(=O)CCC3)c(Cl)c2)c2cnc3cc(F)nn3c21 ; C[C@]1(c2cnn(C(F)F)c2)C[C@@H](C(=O)Nc2cnc(N=S3(=O)CCC3)c(Cl)c2)c2cnc3cc(F)nn3c21]

### Examples 96, 97: (6S,8R)—N-(5-chloro-6-((1-oxido-1)⁶-thietan-1-ylidene)amino)pyridin-3-yl)-8-(1-(difluoromethyl)-1H-pyrazol-4-yl)-2-fluoro-8-methyl-7,8-dihydro-6H-cyclopenta[e]pyrazolo[1,5-a]pyrimidine-6-carboxamide; (6R,8R)—N-(5-chloro-6-((1-oxido-1%⁶-thietan-1-ylidene)amino)pyridin-3-yl)-8-(1-(difluoromethyl)-1H-pyrazol-4-yl)-2-fluoro-8-methyl-7,8-dihydro-6H-cyclopenta[e]pyrazolo[1,5-a]pyrimidine-6-carboxamide

The relative and absolute stereochemistry of each example was not determined.

### Step 1: (S)-2-fluoro-8-methyl-8-(1-((2-(trimethylsilyl)ethoxy)methyl)-1H-pyrazol-4-yl)-7,8-dihydro-6H-cyclopenta[e]pyrazolo[1,5-a]pyrimidine (A25-1-P1) and (R)-2-fluoro-8-methyl-8-(1-((2-(trimethylsilyl)ethoxy)methyl)-1H-pyrazol-4-yl)-7,8-dihydro-6H-cyclopenta[e]pyrazolo[1,5-a]pyrimidine (A25-1-P2)

3 g of 2-fluoro-8-methyl-8-(1-((2-(trimethylsilyl)ethoxy)methyl)-1H-pyrazol-4-yl)-7,8-dihydro-6H-cyclopenta[e]pyrazolo[1,5-a]pyrimidine (A6-6) were submitted to chiral HPLC purification (Column: daicel chiral pakia, 250*50 mm, 10 um; Mobile phase A: C02, Mobile phase B: IPA (0.1% NH3H₂O); Flow rate: 200 g/min; Gradient: isocratic 25% Mobile Phase B; Wavelength: 220 nm; Column temperature (° C.): 40; System back pressure:100 bar. RT1 (min): 3.68; RT2 (min): 6.71, Sample Solvent: IPA-preparative; Injection Volume: 8.5 mL. Number of Runs: 6). The first eluting isomer was concentrated and lyophilized to afford A25-1-P1 (1.3 g) as a light yellow oil, LC-Ms: m/z 388 [M+H]⁺. The second eluting isomer was concentrated and lyophilized to afford A25-1-P2 (1.32 g) as a light yellow oil, LC-Ms: m/z 388 [M+H]⁺.

### Step 2: Methyl (8R)-2-fluoro-8-methyl-8-(1H-pyrazol-4-yl)-7,8-dihydro-6H-cyclopenta[e]pyrazolo[1,5-a]pyrimidine-6-carboxylate

Using (R)-2-fluoro-8-methyl-8-(1-((2-(trimethylsilyl)ethoxy)methyl)-1H-pyrazol-4-yl)-7,8-dihydro-6H-cyclopenta[e]pyrazolo[1,5-a]pyrimidine (A25-1-P2) and following sequentially the reaction conditions described in Method A6 step 7, step 8 and step 9, the title compound was obtained as a yellow oil.

### Step 3: (8R)-8-(1-(difluoromethyl)-1H-pyrazol-4-yl)-2-fluoro-8-methyl-7,8-dihydro-6H-cyclopenta[e]pyrazolo[1,5-a]pyrimidine-6-carboxylic acid (A25-3-P2)

Using methyl (8R)-2-fluoro-8-methyl-8-(1H-pyrazol-4-yl)-7,8-dihydro-6H-cyclopenta[e]pyrazolo[1,5-a]pyrimidine-6-carboxylate and following sequentially the reaction conditions described in Method A7 step 1 and step 2, the title compound (A25-3-P2) was obtained as a yellow oil.

### Step 4: 1-((3-chloro-5-nitropyridin-2-yl)imino)-1λ⁶-thietane 1-oxide

To a stirred solution of 2,3-dichloro-5-nitropyridine (200 mg, 1.0 mmol) and 1-imino-1λ⁶-thietane 1-oxide (109 mg, 1.0 mmol) in dioxane (6 mL) were added Pd₂(dba)₃ (95 mg, 104 μmol), XantPhos (120 mg, 207 μmol) and Cs₂CO₃ (1.0 g, 3.1 mmol) under nitrogen atmosphere. The resulting mixture was stirred at 110° C. for 12 h. The mixture was cooled to 25° C. The reaction mixture was quenched by the addition of water (20 mL). The resulting mixture was extracted with ethyl acetate (3×20 mL). The combined organic layers were dried over anhydrous sodium sulfate and concentrated under reduced pressure. The residue was purified by column chromatography on silica gel using 50% petroleum ether and 50% ethyl acetate as eluent to afford the title compound (140 mg, 51% yield) as a yellow oil. LC-MS: m/z 262 [M+H]⁺.

### Step 5: 1-((5-amino-3-chloropyridin-2-yl)imino)-1λ⁶-thietane 1-oxide

To a solution of 1-((3-chloro-5-nitropyridin-2-yl)imino)-1λ⁶-thietane 1-oxide (120 mg, 459 μmol) in ethanol (7 mL) and water (2 mL) were added Fe (128 mg, 2.3 mmol) and NH₄Cl (123 mg, 2.3 mmol). The resulting mixture was stirred at 80° C. for 2 h. The solids were filtered out. The filtrate was diluted with water (10 mL) and extracted with ethyl acetate (3×20 mL). The combined organic layers were dried over anhydrous sodium sulfate and concentrated under reduced pressure to afford the title compound (75 mg, 70% yield) as a brown solid. LC-MS: m/z 232[M+H]⁺.

### Step 6: Example 96 and Example 97

In analogy to Method A1 step 8 but using (8R)-8-(1-(difluoromethyl)-1H-pyrazol-4-yl)-2-fluoro-8-methyl-7,8-dihydro-6H-cyclopenta[e]pyrazolo[1,5-a]pyrimidine-6-carboxylic acid (A25-3-P2, 75.8 mg, 215.8 μmol) and 1-((5-amino-3-chloropyridin-2-yl)imino)-1)⁶-thietane 1-oxide (50 mg, 215.8 μmol), the diastercomeric title compounds were obtained: trans isomer Example 96 (3.1 mg, 2.5% yield) as an off-white solid and cis-isomer Example 97 (3.4 mg, 2.8% yield) as a white solid.

Example 96: ¹HNMR (400 MHz, DMSO-d₆) δ:10.45 (s, 1H), 8.63 (s, 1H), 8.25 (d, J=2.4 Hz, 1H), 8.19 (s, 1H), 8.14 (d, J=2.4 Hz, 1H), 7.82 (s, 1H), 7.72 (t, J=58.8 Hz, 1H), 6.58 (d, J=4.8 Hz, 1H), 4.28-4.46 (m, 5H), 2.85-2.88 (m, 1H), 2.50-2.55 (m, 1H), 2.23-2.39 (m, 2H), 1.98 (s, 3H). LC-MS: m/z 565 [M+H]⁺.

Example 97: ¹HNMR (400 MHz, DMSO-d₆) δ:10.44 (s, 1H), 8.69 (s, 1H), 8.23 (d, J=2.4 Hz, 1H), 8.18 (s, 1H), 8.09 (d, J=2.4 Hz, 1H), 7.75 (s, 1H), 7.73 (t, J=58.8 Hz, 1H), 6.56 (d, J=5.2 Hz, 1H), 4.31-4.47 (m, 5H), 2.77-2.83 (m, 1H), 2.65-2.70 (m, 1H), 2.23-2.35 (m, 2H), 1.98 (s, 3H). LC-MS: m/z 565 [M+H]⁺.

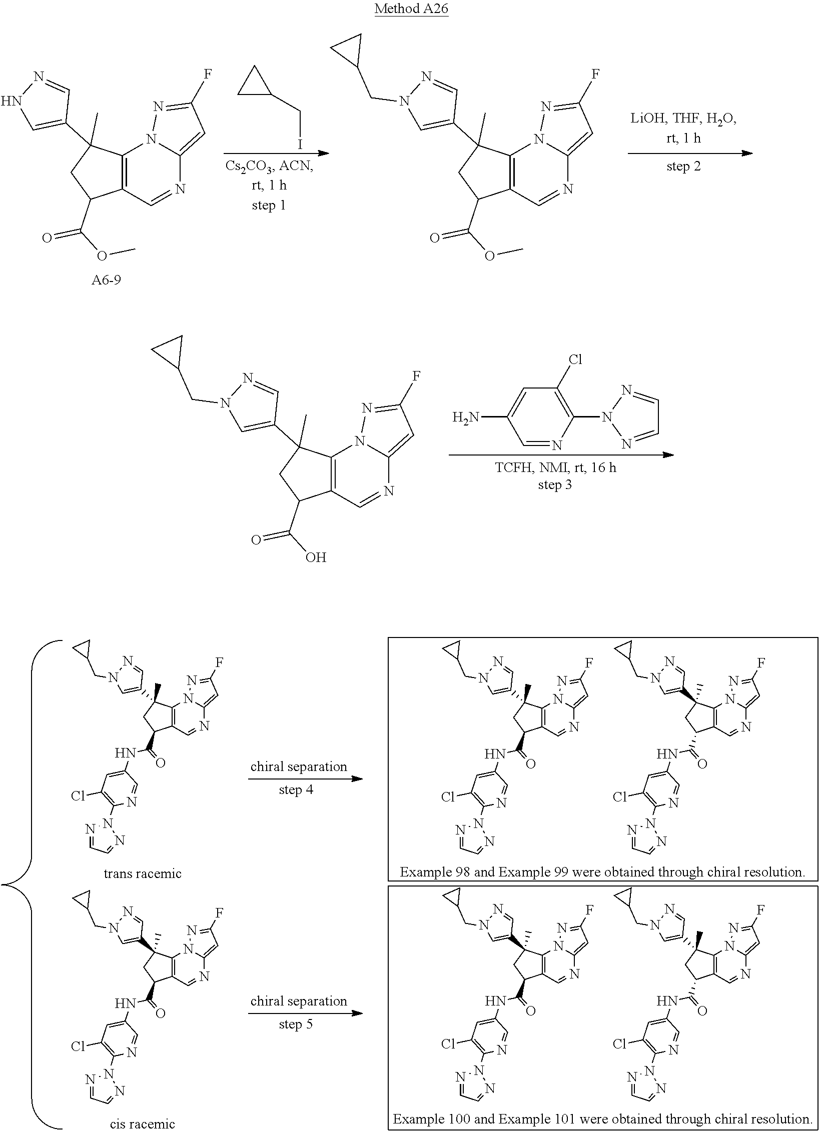

### Examples 98, 99: (6S,8R)—N-(5-chloro-6-(2H-1,2,3-triazol-2-yl)pyridin-3-yl)-8-(1-(cyclopropylmethyl)-1H-pyrazol-4-yl)-2-fluoro-8-methyl-7,8-dihydro-6H-cyclopenta[e]pyrazolo[1,5-a]pyrimidine-6-carboxamide; (6R,8S)—N-(5-chloro-6-(2H-1,2,3-triazol-2-yl)pyridin-3-yl)-8-(1-(cyclopropylmethyl)-1H-pyrazol-4-yl)-2-fluoro-8-methyl-7,8-dihydro-6H-cyclopenta[e]pyrazolo[1,5-a]pyrimidine-6-carboxamide

### Examples 100, 101: (6S,8S)—N-(5-chloro-6-(2H-1,2,3-triazol-2-yl)pyridin-3-yl)-8-(1-(cyclopropylmethyl)-1H-pyrazol-4-yl)-2-fluoro-8-methyl-7,8-dihydro-6H-cyclopenta[e]pyrazolo[1,5-a]pyrimidine-6-carboxamide; (6R,8R)—N-(5-chloro-6-(2H-1,2,3-triazol-2-yl)pyridin-3-yl)-8-(1-(cyclopropylmethyl)-1H-pyrazol-4-yl)-2-fluoro-8-methyl-7,8-dihydro-6H-cyclopenta[e]pyrazolo[1,5-a]pyrimidine-6-carboxamide

The relative and absolute stereochemistry of each example was not determined.

### Step 1: Methyl 8-(1-(cyclopropylmethyl)-1H-pyrazol-4-yl)-2-fluoro-8-methyl-7,8-dihydro-6H-cyclopenta[e]pyrazolo[1,5-a]pyrimidine-6-carboxylate

In analogy to Method A6 step 10 but using methyl 2-fluoro-8-methyl-8-(1H-pyrazol-4-yl)-7,8-dihydro-6H-cyclopenta[e]pyrazolo[1,5-a]pyrimidine-6-carboxylate (A6-9, 225 mg, 711 mol), the title compound was obtained as a yellow solid (135 mg, 36% yield). LC-MS (ES, m/z): 370[M+H]⁺.

### Step 2: 8-(1-(cyclopropylmethyl)-1H-pyrazol-4-yl)-2-fluoro-8-methyl-7,8-dihydro-6H-cyclopenta[e]pyrazolo[1,5-a]pyrimidine-6-carboxylic acid

In analogy to Method A7 step 2 but using methyl 8-(1-(cyclopropylmethyl)-1H-pyrazol-4-yl)-2-fluoro-8-methyl-7,8-dihydro-6H-cyclopenta[e]pyrazolo[1,5-a]pyrimidine-6-carboxylate (170 mg, 460 μmol), the crude title compound was obtained as a yellow oil (100 mg, 57% yield). LC-MS (ES, m/z): 356[M+H]⁺.

### Step 3: (trans)-N-(5-chloro-6-(2H-1,2,3-triazol-2-yl)pyridin-3-yl)-8-(1-(cyclopropylmethyl)-1H-pyrazol-4-yl)-2-fluoro-8-methyl-7,8-dihydro-6H-cyclopenta[e]pyrazolo[1,5-a]pyrimidine-6-carboxamide and (cis)-N-(5-chloro-6-(2H-1,2,3-triazol-2-yl)pyridin-3-yl)-8-(1-(cyclopropylmethyl)-1H-pyrazol-4-yl)-2-fluoro-8-methyl-7,8-dihydro-6H-cyclopenta[e]pyrazolo[1,5-a]pyrimidine-6-carboxamide

In analogy to Method A1 step 8 but using 8-(1-(cyclopropylmethyl)-1H-pyrazol-4-yl)-2-fluoro-8-methyl-7,8-dihydro-6H-cyclopenta[e]pyrazolo[1,5-a]pyrimidine-6-carboxylic acid (100 mg, 281 μmol) and 5-chloro-6-(triazol-2-yl)pyridin-3-amine (55 mg, 281 μmol), racemic mixtures of the title compounds were obtained: trans isomer (48 mg, 30% yield) as a white solid, LC-MS (ES, m/z): 533[M+H]⁺; cis-isomer (37 mg, 23% yield) as a white solid, LC-MS (ES, m/z): 533[M+H]⁺.

### Step 4: Separation of Enantiomers to Obtain Example 98 and Example 99

40 mg of (trans)-N-(5-chloro-6-(2H-1,2,3-triazol-2-yl)pyridin-3-yl)-8-(1-(cyclopropylmethyl)-1H-pyrazol-4-yl)-2-fluoro-8-methyl-7,8-dihydro-6H-cyclopenta[e]pyrazolo[1,5-a]pyrimidine-6-carboxamide were submitted to chiral HPLC purification (Column: CHIRALPAK IF, 2*25 cm, 5 μm; Mobile Phase A: MtBE (0.1% FA)-HPLC, Mobile Phase B: EtOH-HPLC; Flow rate: 14 mL/min; Gradient: isocratic 50% Mobile Phase B; Wave Length: 254/220 nm nm; RT1 (min): 7.463; RT2 (min): 12.304; Sample Solvent: EtOH-HPLC; Injection Volume: 2.5 mL; Number Of Runs: 3). The second eluting isomer was concentrated and lyophilized to afford Example 98 (8.0 mg, 19% yield) as a white solid. The first eluting isomer was concentrated and lyophilized to afford Example 99 (8.2 mg, 19% yield) as a white solid.

Example 98: ¹H NMR (400 MHz, Chloroform-d) δ 8.74 (d, J=1.2 Hz, 1H), 8.70 (s, 1H), 8.57 (s, 1H), 7.88-8.23 (m, 2H), 7.96 (s, 2H), 6.31 (d, J=4.8 Hz, 1H), 4.56-4.66 (m, 1H), 4.04-4.19 (m, 2H), 3.22-3.43 (m, 1H), 2.61-2.71 (m, 1H), 2.13 (s, 3H), 1.30-1.37 (m, 1H), 0.70-0.76 (m, 2H), 0.44-0.49 (m, 2H). LC-MS (ES, m/z): 533[M+H]⁺.

Example 99: ¹H NMR (400 MHz, Chloroform-d) δ 8.74 (s, 1H), 8.70 (s, 1H), 8.57 (s, 1H), 7.90-8.26 (m, 2H), 7.96 (s, 2H), 6.31 (d, J=5.2 Hz, 1H), 4.55-4.70 (m, 1H), 4.04-4.20 (m, 2H), 3.28-3.40 (m, 1H), 2.61-2.71 (m, 1H), 2.13 (s, 3H), 1.30-1.39 (m, 1H), 0.70-0.76 (m, 2H), 0.45-0.49 (m, 2H). LC-MS (ES, m/z): 533[M+H]⁺.

### Step 5: Separation of Enantiomers to Obtain Example 100 and Example 101

30 mg of (cis)-N-(5-chloro-6-(2H-1,2,3-triazol-2-yl)pyridin-3-yl)-8-(1-(cyclopropylmethyl)-1H-pyrazol-4-yl)-2-fluoro-8-methyl-7,8-dihydro-6H-cyclopenta[e]pyrazolo[1,5-a]pyrimidine-6-carboxamide were submitted to chiral HPLC purification (Column: CHIRALPAK IF, 2*25 cm, 5 m; Mobile Phase A: MtBE (0.1% FA)-HPLC, Mobile Phase B: EtOH-HPLC; Flow rate: 14 mL/min; Gradient: isocratic 50% Mobile Phase B; Wave Length: 254/220 nm nm; RT1 (min): 6.303; RT2 (min): 12.389; Sample Solvent: EtOH-HPLC; Injection Volume: 2.0 mL; Number Of Runs: 4). The second eluting isomer was concentrated and lyophilized to afford Example 100 (4.1 mg, 13% yield) as a white solid. The first eluting isomer was concentrated and lyophilized to afford Example 101 (4.2 mg, 13% yield) as a white solid.

Example 100: ¹H NMR (400 MHz, Chloroform-d) δ 8.62 (s, 1H), 8.53 (s, 1H), 8.37-8.48 (m, 2H), 7.94 (s, 2H), 7.75 (s, 1H), 7.73 (s, 1H), 6.29 (d, J=5.2 Hz, 1H), 4.45-4.52 (m, 1H), 3.93-4.08 (m, 2H), 2.99-3.07 (m, 1H), 2.85-2.95 (m, 1H), 2.02 (s, 3H), 1.20-1.30 (m, 1H), 0.58-0.68 (m, 2H), 0.32-0.41 (m, 2H). LC-MS (ES, m/z): 533 [M+H]⁺.

Example 101: ¹H NMR (400 MHz, Chloroform-d) δ 8.80 (s, 1H), 8.65 (s, 1H), 8.46-8.56 (m, 2H), 7.94 (s, 2H), 7.81-7.86 (m, 2H), 6.31 (d, J=4.8 Hz, 1H), 4.53-4.58 (m, 1H), 4.03-4.10 (m, 2H), 2.87-3.07 (m, 2H), 2.02 (s, 3H), 1.28 (s, 1H), 0.63-0.70 (m, 2H), 0.37-0.43 (m, 2H). LC-MS (ES, m/z): 533 [M+H]⁺.

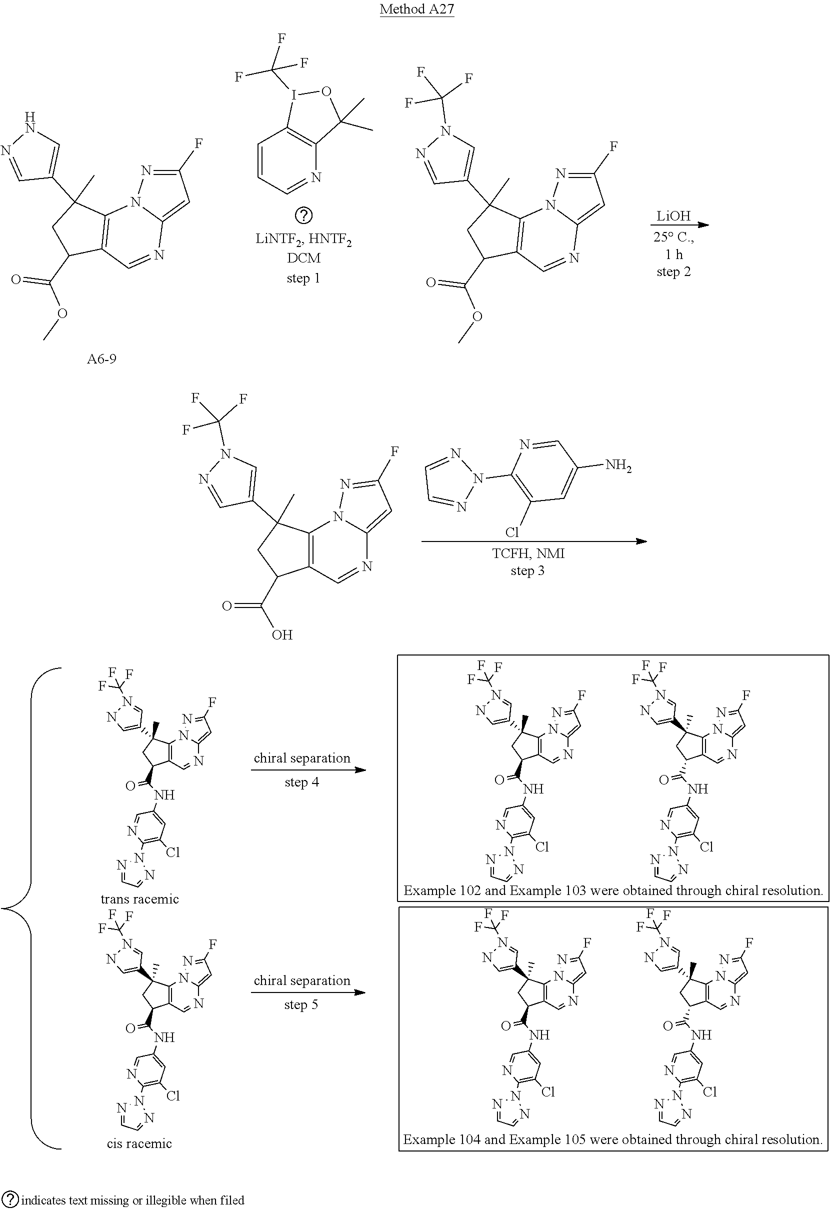

### Examples 102, 103: (6S,8R)—N-(5-chloro-6-(2H-1,2,3-triazol-2-yl)pyridin-3-yl)-2-fluoro-8-methyl-8-(1-(trifluoromethyl)-1H-pyrazol-4-yl)-7,8-dihydro-6H-cyclopenta[e]pyrazolo[1,5-a]pyrimidine-6-carboxamide; (6R,8S)—N-(5-chloro-6-(2H-1,2,3-triazol-2-yl)pyridin-3-yl)-2-fluoro-8-methyl-8-(1-(trifluoromethyl)-1H-pyrazol-4-yl)-7,8-dihydro-6H-cyclopenta[e]pyrazolo[1,5-a]pyrimidine-6-carboxamide

### Examples 104, 105: (6S,8S)—N-(5-chloro-6-(2H-1,2,3-triazol-2-yl)pyridin-3-yl)-2-fluoro-8-methyl-8-(1-(trifluoromethyl)-1H-pyrazol-4-yl)-7,8-dihydro-6H-cyclopenta[e]pyrazolo[1,5-a]pyrimidine-6-carboxamide; (6R,8R)—N-(5-chloro-6-(2H-1,2,3-triazol-2-yl)pyridin-3-yl)-2-fluoro-8-methyl-8-(1-(trifluoromethyl)-1H-pyrazol-4-yl)-7,8-dihydro-6H-cyclopenta[e]pyrazolo[1,5-a]pyrimidine-6-carboxamide

The relative and absolute stereochemistry of each example was not determined.

### Step 1: Methyl 2-fluoro-8-methyl-8-(1-(trifluoromethyl)-1H-pyrazol-4-yl)-7,8-dihydro-6H-cyclopenta[e]pyrazolo[1,5-a]pyrimidine-6-carboxylate

To a stirred solution of methyl 2-fluoro-8-methyl-8-(1H-pyrazol-4-yl)-7,8-dihydro-6H-cyclopenta[e]pyrazolo[1,5-a]pyrimidine-6-carboxylate (1.0 g, 3.2 mmol) in DCM (20 mL) was added lithium bis((trifluoromethyl)sulfonyl)amide (91 mg, 317 μmol) and 1,1,1-trifluoro-N-((trifluoromethyl)sulfonyl)methanesulfonamide (892 mg, 3.2 mmol), followed by the addition of 3,3-dimethyl-1-(trifluoromethyl)-1,3-dihydro-3-benzo[d][1,2]iodaoxole (1.1 g, 3.2 mmol) in portions. The reaction mixture was stirred at 40° C. for 2 h. The reaction was quenched with water (20 mL). The resulting mixture was extracted with DCM (3×20 mL). The combined organic layers were dried over anhydrous sodium sulfate and concentrated under reduced pressure. The residue was applied on a silica gel column and eluted with EtOAc/PE (1:1) to afford the title compound (200 mg, 16% yield) as a yellow solid. LC-MS: m/z 384 [M+H]⁺.

### Step 2: 2-fluoro-8-methyl-8-(1-(trifluoromethyl)-1H-pyrazol-4-yl)-7,8-dihydro-6H-cyclopenta[e]pyrazolo[1,5-a]pyrimidine-6-carboxylic acid

In analogy to Method A7 step 2 but using methyl 2-fluoro-8-methyl-8-(1-(trifluoromethyl)-1H-pyrazol-4-yl)-7,8-dihydro-6H-cyclopenta[e]pyrazolo[1,5-a]pyrimidine-6-carboxylate (210 mg, 547.8 μmol), the crude title compound was obtained as a yellow oil (90 mg) as a yellow solid. LC-MS: m/z 370 [M+H]⁺.

### Step 3: (trans)-N-(5-chloro-6-(2H-1,2,3-triazol-2-yl)pyridin-3-yl)-2-fluoro-8-methyl-8-(1-(trifluoromethyl)-1H-pyrazol-4-yl)-7,8-dihydro-6H-cyclopenta[e]pyrazolo[1,5-a]pyrimidine-6-carboxamide and (cis)-N-(5-chloro-6-(2H-1,2,3-triazol-2-yl)pyridin-3-yl)-2-fluoro-8-methyl-8-(1-(trifluoromethyl)-1H-pyrazol-4-yl)-7,8-dihydro-6H-cyclopenta[e]pyrazolo[1,5-a]pyrimidine-6-carboxamide

In analogy to Method A1 step 8 but using 2-fluoro-8-methyl-8-(1-(trifluoromethyl)-1H-pyrazol-4-yl)-7,8-dihydro-6H-cyclopenta[e]pyrazolo[1,5-a]pyrimidine-6-carboxylic acid (90 mg, 244 μmol) and 5-chloro-6-(2H-1,2,3-triazol-2-yl)pyridin-3-amine (48 mg, 244 μmol), racemic mixtures of the title compounds were obtained: trans isomer (13.5 mg, 10% yield) as an off-white solid, LC-MS: m/z 547 [M+H]⁺; cis-isomer (9.9 mg, 7.4% yield) as a white solid, LC-MS: m/z 547 [M+H]⁺.

### Step 4: Separation of Enantiomers to Obtain Example 102 and Example 103

13 mg of (trans)-N-(5-chloro-6-(2H-1,2,3-triazol-2-yl)pyridin-3-yl)-2-fluoro-8-methyl-8-(1-(trifluoromethyl)-1H-pyrazol-4-yl)-7,8-dihydro-6H-cyclopenta[e]pyrazolo[1,5-a]pyrimidine-6-carboxamide were submitted to chiral HPLC purification (Column: CHIRAL ART Amylose-SA, 2*25 cm, 5 m; Mobile Phase A: Hex: DCM=3:1(0.1% FA)-HPLC, Mobile Phase B: EtOH-HPLC; Flow rate: 20 ML/MIN mL/min; Gradient: isocratic 50% Mobile Phase B; Wave Length: 254/220 nm nm; RT1 (min): 4.891; RT2 (min): 6.658; Sample Solvent: EtOH-HPLC; Injection Volume: 1.0 mL; Number Of Runs: 2). The first eluting isomer was concentrated and lyophilized to afford Example 103 (3.0 mg, 23% yield) as a white solid. The second eluting isomer was concentrated and lyophilized to afford Example 102 (2.4 mg, 18% yield) as a white solid.

Example 102: ¹HNMR (400 MHz, DMSO-d₆) δ: 11.12 (s, 1H), 8.73 (d, J=2.4 Hz, 1H), 8.72 (s, 1H), 8.60 (d, J=2.4 Hz, 111), 8.49 (s, 1H), 8.17 (s, 2H), 8.00 (s, 111), 6.60 (d, J=4.8 Hz, 1H), 4.55-4.59 (m, 1H), 2.90-2.96 (m, 1H), 2.58-2.63 (m, 1H), 2.00 (s, 3H). LC-MS: m/z 547 [M+H]⁺.

Example 103: ¹HNMR (400 MHz, DMSO-d₆) δ: 11.11 (s, 1H), 8.73 (d, J=2.0 Hz, 1H), 8.72 (s, 1H), 8.60 (d, J=2.0 Hz, 1H), 8.49 (s, 1H), 8.17 (s, 2H), 8.00 (s, 1H), 6.60 (d, J=4.8 Hz, 1H), 4.55-4.59 (m, 1H), 2.90-2.96 (m, 1H), 2.58-2.63 (m, 1H), 2.00 (s, 3H). LC-MS: m/z 547 [M+H]⁺.

### Step 5: Separation of Enantiomers to Obtain Example 104 and Example 105

9 mg of (cis)-N-(5-chloro-6-(2H-1,2,3-triazol-2-yl)pyridin-3-yl)-2-fluoro-8-methyl-8-(1-(trifluoromethyl)-1H-pyrazol-4-yl)-7,8-dihydro-6H-cyclopenta[e]pyrazolo[1,5-a]pyrimidine-6-carboxamide were submitted to chiral HPLC purification (Column: CHIRAL ART Amylose-SA, 2*25 cm, 5 m; Mobile Phase A: Hex: DCM=3:1(0.1% FA)-HPLC, Mobile Phase B: EtOH-HPLC; Flow rate: 20 ML/MIN mL/min; Gradient: isocratic 20% Mobile Phase B; Wave Length: 254/220 nm nm; RT1 (min): 4.175; RT2 (min): 4.838; Sample Solvent: EtOH-HPLC; Injection Volume: 1.0 mL; Number Of Runs: 2). The first eluting isomer was concentrated and lyophilized to afford Example 105 (1.1 mg, 12% yield) as a white solid. The second eluting isomer was concentrated and lyophilized to afford Example 104 (2.9 mg, 32% yield) as a white solid.

Example 104: ¹HNMR (400 MHz, DMSO-d₆) δ:11.09 (s, 1H), 8.76 (s, 1H), 8.73 (d, J=2.0 Hz, 1H), 8.56 (d, J=2.0 Hz, 1H), 8.47 (s, 1H), 8.17 (s, 2H), 7.92 (s, 1H), 6.57 (d, J=4.8 Hz, 1H), 4.55-4.59 (m, 1H), 2.83-2.88 (m, 1H), 2.72-2.77 (m, 1H), 1.91 (s, 3H). LC-MS: m/z 547 [M+H]⁺.

Example 105: ¹HNMR (400 MHz, DMSO-d₆) δ: 11.09 (s, 1H), 8.76 (s, 1H), 8.73 (d, J=2.0 Hz, 1H), 8.56 (d, J=2.0 Hz, 1H), 8.47 (s, 1H), 8.17 (s, 2H), 7.92 (s, 1H), 6.57 (d, J=4.8 Hz, 1H), 4.55-4.58 (m, 1H), 2.83-2.88 (m, 1H), 2.72-2.77 (m, 1H), 1.91 (s, 3H). LC-MS: m/z 547 [M+H]⁺.

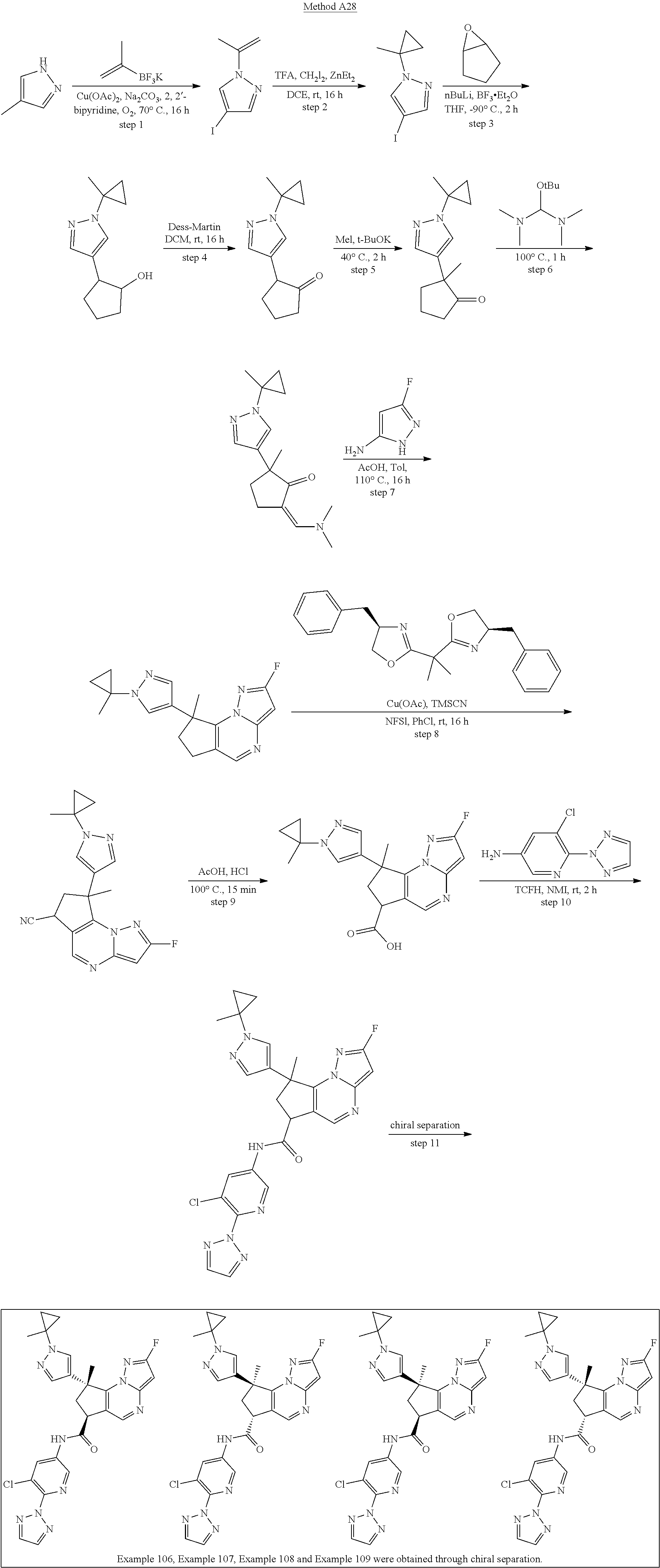

### Examples 106, 107, 108, 109: (6S,8R)—N-(5-chloro-6-(2H-1,2,3-triazol-2-yl)pyridin-3-yl)-2-fluoro-8-methyl-8-(1-(1-methylcyclopropyl)-1H-pyrazol-4-yl)-7,8-dihydro-6H-cyclopenta[e]pyrazolo[1,5-a]pyrimidine-6-carboxamide; (6R,8S)—N-(5-chloro-6-(2H-1,2,3-triazol-2-yl)pyridin-3-yl)-2-fluoro-8-methyl-8-(1-(1-methylcyclopropyl)-1H-pyrazol-4-yl)-7,8-dihydro-6H-cyclopenta[e]pyrazolo[1,5-a]pyrimidine-6-carboxamide; (6S,8S)—N-(5-chloro-6-(2H-1,2,3-triazol-2-yl)pyridin-3-yl)-2-fluoro-8-methyl-8-(1-(1-methylcyclopropyl)-1H-pyrazol-4-yl)-7,8-dihydro-6H-cyclopenta[e]pyrazolo[1,5-a]pyrimidine-6-carboxamide; (6R,8R)—N-(5-chloro-6-(2H-1,2,3-triazol-2-yl)pyridin-3-yl)-2-fluoro-8-methyl-8-(1-(I-methylcyclopropyl)-1H-pyrazol-4-yl)-7,8-dihydro-6H-cyclopenta[e]pyrazolo[1,5-a]pyrimidine-6-carboxamide

The relative and absolute stereochemistry of each example was not determined.

### Step 1: 4-iodo-1-(prop-1-en-2-yl)-1H-pyrazole

To a stirred solution of 4-iodo-1H-pyrazole (20 g, 103.1 mmol) in DCE (70 mL) were added diacetoxycopper (18.73 g, 103.1 mmol), 2,2′-bipyridine (32.2 g, 206.2 mmol), sodium carbonate (21.9 g, 206.2 mmol) and potassium trifluoro(prop-1-en-2-yl)borate (30.5 g, 206.2 mmol). The mixture was stirred at 70° C. for 2 h under oxygen atmosphere. The reaction was quenched with water (350 mL). The resulting solution was extracted with DCM (2×350 mL). The combined organic layers were dried over anhydrous sodium sulfate and concentrated under reduced pressure. The residue was applied on a silica gel column and eluted with PE/EtOAc (3:1) to give the title compound (16.6 g, 61% yield) as a yellow oil. LC-MS (ES, m/z): 235[M+H]⁺.

### Step 2: 4-iodo-1-(1-methylcyclopropyl)-1H-pyrazole

To a solution of 2,2,2-trifluoroacetic acid (36.5 g, 320.4 mmol) in DCE (200 mL) was added a solution of diethylzinc (1M, 320 mL) at 0° C. under nitrogen. The mixture was stirred at 0° C. for 20 min. A solution of diiodomethane (85.8 g, 320.4 mmol) in DCE (200 mL) was added, and the mixture was stirred at 0° C. for 20 min under nitrogen. A solution of 4-iodo-1-(prop-1-en-2-yl)-1H-pyrazole (15 g, 64.1 mmol) in DCE (200 mL) was added, and the mixture was stirred at 25° C. for 16 h under nitrogen. The reaction was quenched with water (50 mL). The resulting mixture was extracted with DCM (2×30 mL). The combined organic layers were dried over anhydrous sodium sulfate and concentrated under reduced pressure. The residue was applied on a silica gel column and eluted with PE/EtOAc (3:1) to give the title compound (7.5 g, 47% yield) as a yellow oil. LC-MS (ES, m/z): 249[M+H]⁺.

### Step 3: 2-(1-(1-methylcyclopropyl)-1H-pyrazol-4-yl)cyclopentan-1-ol

In analogy to Method A6 step 2 but using 4-iodo-1-(1-methylcyclopropyl)-1H-pyrazole (7.9 g, 31.8 mmol), the title compound was obtained as a yellow oil (0.9 g, 13% yield) as a yellow oil. LC-MS: m/z 207 [M+H]⁺.

### Step 4: 2-(1-(1-methylcyclopropyl)-1H-pyrazol-4-yl)cyclopentan-1-one

In analogy to Method A10 step 2 but using 2-(1-(1-methylcyclopropyl)-1H-pyrazol-4-yl)cyclopentan-1-ol (1.8 g, 8.7 mmol) purification of the product by prep-HPLC, the title compound was obtained as a yellow oil (880 mg, 49% yield). ¹H NMR (400 MHz, DMSO-d₆) δ: 7.61 (s, 1H), 7.28 (s, 1H), 3.20-3.25 (m, 1H), 2.13-2.38 (m, 3H), 1.78-2.01 (m, 3H), 1.52 (s, 3H), 1.10-1.14 (m, 2H), 0.82-0.88 (m, 2H). LC-MS: m/z 205 [M+H]⁺.

### Step 5: 2-methyl-2-(1-(1-methylcyclopropyl)-1H-pyrazol-4-yl)cyclopentan-1-one

In analogy to Method A1 step 3 but using 2-(1-(1-methylcyclopropyl)-1H-pyrazol-4-yl)cyclopentan-1-one (830 mg, 4.1 mmol), and purification of the product by silica gel column chromatography eluting with PE/EA (1:1), the title compound was obtained as a yellow oil (500 mg, 39% yield). ¹H NMR (400 MHz, DMSO-d₆) δ: 7.61 (s, 1H), 7.29 (s, 1H), 2.19-2.35 (m, 3H), 1.79-2.01 (m, 3H), 1.51 (s, 3H), 1.22 (s, 3H), 1.08-1.16 (m, 2H), 0.81-0.87 (m, 2H). LC-MS: m/z 219 [M+H]⁺.

### Step 6: 5-((dimethylamino)methylene)-2-methyl-2-(1-(1-methylcyclopropyl)-1H-pyrazol-4-yl)cyclopentan-1-one

In analogy to Method A1 step 4 but using 2-methyl-2-(1-(1-methylcyclopropyl)-1H-pyrazol-4-yl)cyclopentan-1-one (450 mg, 2.1 mmol) in 1-tert-butoxy-N,N,N′,N′-tetramethyl-methanediamine (719 mg, 4.2 mmol) and stirring at 100° C. for 1, the crude title compound was obtained as a yellow oil (500 mg, 88% yield) was used in the next step without further purification. LC-MS: m/z 274 [M+H]⁺.

### Step 7: 2-fluoro-8-methyl-8-(1-(1-methylcyclopropyl)-1H-pyrazol-4-yl)-7,8-dihydro-6H-cyclopenta[e]pyrazolo[1,5-a]pyrimidine

In analogy to Method A1 step 5 but using 5-((dimethylamino)methylene)-2-methyl-2-(1-(1-methylcyclopropyl)-1H-pyrazol-4-yl)cyclopentan-1-one (500 mg, 1.8 mmol), the title compound was obtained as a yellow oil (480 mg, 84% yield). ¹H NMR (400 MHz, Chloroform-d) δ 8.28 (s, 1H), 7.40 (s, 1H), 7.34 (s, 1H), 6.12 (d, J=4.8 Hz, 1H), 2.89-3.02 (m, 2H), 2.47-2.61 (m, 1H), 2.19-2.33 (m, 1H), 1.65 (s, 3H), 1.57 (s, 3H), 1.16-1.25 (m, 2H), 0.82-0.90 (m, 2H). LC-MS: m/z 312 [M+H]⁺.

### Step 8: 2-fluoro-8-methyl-8-(1-(1-methylcyclopropyl)-1H-pyrazol-4-yl)-7,8-dihydro-6H-cyclopenta[e]pyrazolo[1,5-a]pyrimidine-6-carbonitrile

In analogy to Method A1 step 6 but using 2-fluoro-8-methyl-8-(1-(1-methylcyclopropyl)-1H-pyrazol-4-yl)-7,8-dihydro-6H-cyclopenta[e]pyrazolo[1,5-a]pyrimidine (520 mg, 1.7 mmol) in chlorobenzene (50 mL), the title compound was obtained as a yellow solid (150 mg, 17% yield). LC-MS: m/z 337 [M+H]⁺.

### Step 9: 2-fluoro-8-methyl-8-(1-(1-methylcyclopropyl)-1H-pyrazol-4-yl)-7,8-dihydro-6H-cyclopenta[e]pyrazolo[1,5-a]pyrimidine-6-carboxylic acid

In analogy to Method A9 step 8 but using 2-fluoro-8-methyl-8-(1-(1-methylcyclopropyl)-1H-pyrazol-4-yl)-7,8-dihydro-6H-cyclopenta[e]pyrazolo[1,5-a]pyrimidine-6-carbonitrile (90 mg, 268 mol), the title compound was obtained as a yellow solid (80 mg, 84% yield). LC-MS: m/z 356 [M+H]⁺.

### Step 10: N-(5-chloro-6-(2H-1,2,3-triazol-2-yl)pyridin-3-yl)-2-fluoro-8-methyl-8-(1-(1-methylcyclopropyl)-1H-pyrazol-4-yl)-7,8-dihydro-6H-cyclopenta[e]pyrazolo[1,5-a]pyrimidine-6-carboxamide

In analogy to Method A1 step 8 but using 2-fluoro-8-methyl-8-(1-(1-methylcyclopropyl)-1H-pyrazol-4-yl)-7,8-dihydro-6H-cyclopenta[e]pyrazolo[1,5-a]pyrimidine-6-carboxylic acid (60 mg, 169 μmol) and 5-chloro-6-(2H-1,2,3-triazol-2-yl)pyridin-3-amine (40 mg, 203 μmol), a mixture of all stereoisomers of the title compound (27 mg, 30% yield) was obtained as a white solid. LC-MS: m/z 533 [M+H]⁺.

### Step 11: Separation of Enantiomers to Obtain Example 106, Example 107, Example 108, and Example 109

27 mg of N-(5-chloro-6-(2H-1,2,3-triazol-2-yl)pyridin-3-yl)-2-fluoro-8-methyl-8-(1-(1-methylcyclopropyl)-1H-pyrazol-4-yl)-7,8-dihydro-6H-cyclopenta[e]pyrazolo[1,5-a]pyrimidine-6-carboxamide were submitted to chiral HPLC purification (Column: CHIRALPAK IF, 2*25 cm, 5 μm; Mobile Phase A: Hex: DCM=3:1(0.1% FA)-HPLC, Mobile Phase B: EtOH-HPLC; Flow rate: 20 mL/min; Gradient: isocratic 50% Mobile Phase B; Wave Length: 254/220 nm nm; RT1 (min): 5.153; RT2 (min): 7.491; Sample Solvent: EtOH-HPLC; Injection Volume: 1.5 mL; Number Of Runs: 3). The fourth eluting isomer was concentrated and lyophilized to afford Example 106 (7.1 mg, 26% yield) as a white solid. The first eluting isomer was concentrated and lyophilized to afford Example 109 (1.2 mg, 4% yield) as a white solid. The third eluting isomer and the second eluting isomer was concentrated and lyophilized to afford a mixture, which was submitted to chiral HPLC purification (Column: CHIRALPAK ID, 2*25 cm, 5 m; Mobile Phase A: Hex (0.1% FA)-HPLC, Mobile Phase B: EtOH: DCM=1:1-HPLC; Flow rate: 20 mL/min; Gradient: isocratic 70% Mobile Phase B; Wave Length: 254/220 nm nm; RT1 (min): 4.131; RT2 (min): 5.522; Sample Solvent: EtOH-HPLC; Injection Volume: 1.5 mL; Number Of Runs: 2). The second eluting isomer was concentrated and lyophilized to afford Example 107 (4.7 mg, 17% yield) as a white solid. The first eluting isomer was concentrated and lyophilized to afford Example 108 (1.2 mg, 4% yield) as a white solid.

Example 106: ¹H NMR (400 MHz, Chloroform-d) δ: 8.84 (s, 1H), 8.75 (s, 1H), 8.58 (s, 1H), 8.18 (s, 1H), 7.95 (s, 2H), 6.32 (d, J=5.2 Hz, 1H), 4.69-4.84 (m, 1H), 3.38-3.66 (m, 1H), 2.52-2.63 (m, 1H), 2.10 (s, 3H), 1.74 (s, 3H), 1.35-1.39 (m, 2H), 1.10-1.25 (m, 2H). LC-MS (ES, m/z): 533[M+H]⁺.

Example 107: ¹H NMR (400 MHz, Chloroform-d) δ: 9.05 (s, 1H), 8.68 (s, 1H), 8.50-8.56 (m, 2H), 7.95 (s, 2H), 7.63 (s, 1H), 7.44 (s, 1H), 6.29 (d, J=4.8 Hz, 1H), 4.38-4.43 (m, 1H), 3.00-3.04 (m, 1H), 2.65-2.72 (m, 1H), 2.08 (s, 3H), 1.55 (s, 3H), 1.12-1.23 (m, 2H), 0.82-0.94 (m, 2H). LC-MS (ES, m/z): 533[M+H]⁺.

Example 108: ¹H NMR (400 MHz, Chloroform-d) δ: 8.35-8.67 (m, 4H), 7.94 (s, 2H), 7.69-7.81 (m, 2H), 6.31 (d, J=4.0 Hz, 1H), 4.50-4.56 (m, 1H), 2.74-3.21 (m, 2H), 2.02 (s, 3H), 1.60 (s, 3H), 1.25-1.30 (m, 2H), 0.90-0.98 (m, 2H). LC-MS (ES, m/z): 533[M+H]⁺.

Example 109: ¹H NMR (400 MHz, Chloroform-d) δ: 8.66 (s, 1H), 8.52-8.56 (m, 2H), 7.79-7.99 (m, 3H), 7.75 (s, 1H), 6.32 (d, J=4.8 Hz, 1H), 4.50-4.56 (m, 1H), 2.82-3.06 (m, 2H), 1.99 (s, 3H), 1.61 (s, 3H), 1.20-1.33 (m, 2H), 0.94-1.01 (m, 2H). LC-MS (ES, n/z): 533[M+H]⁺.

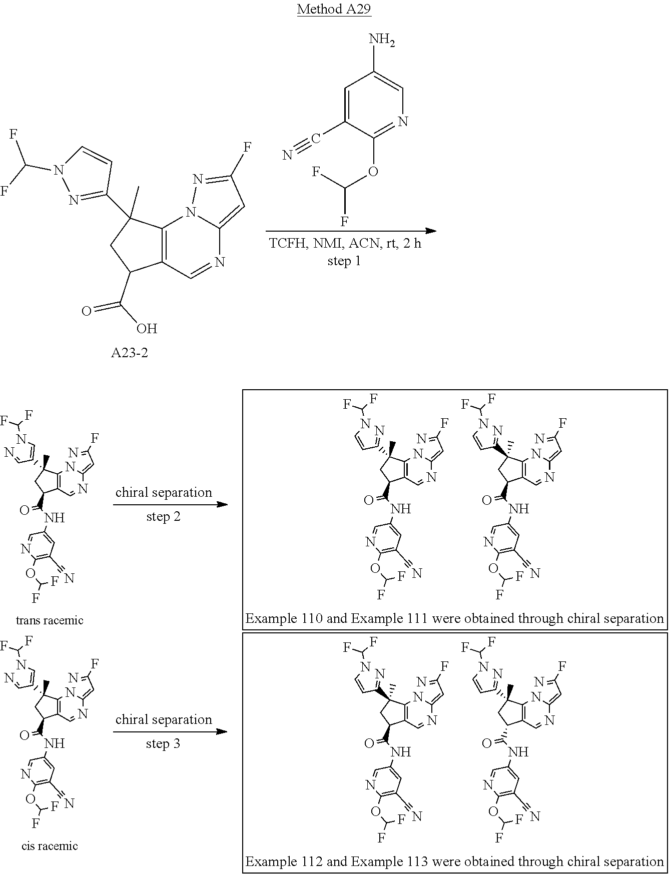

### Examples 110, 111: Single enantiomers obtained from a racemic mixture containing (6S,8S)—N-(5-cyano-6-(difluoromethoxy)pyridin-3-yl)-8-(1-(difluoromethyl)-1H-pyrazol-3-yl)-2-fluoro-8-methyl-7,8-dihydro-6H-cyclopenta[e]pyrazolo[1,5-a]pyrimidine-6-carboxamide; (6R,8R)—N-(5-cyano-6-(difluoromethoxy)pyridin-3-yl)-8-(1-(difluoromethyl)-1H-pyrazol-3-yl)-2-fluoro-8-methyl-7,8-dihydro-6H-cyclopenta[e]pyrazolo[1,5-a]pyrimidine-6-carboxamide

### Examples 112, 113: Single enantiomers obtained from a racemic mixture containing (6S,8R)—N-(5-cyano-6-(difluoromethoxy)pyridin-3-yl)-8-(1-(difluoromethyl)-1H-pyrazol-3-yl)-2-fluoro-8-methyl-7,8-dihydro-6H-cyclopenta[e]pyrazolo[1,5-a]pyrimidine-6-carboxamide; (6R,8S)—N-(5-cyano-6-(difluoromethoxy)pyridin-3-yl)-8-(1-(difluoromethyl)-1H-pyrazol-3-yl)-2-fluoro-8-methyl-7,8-dihydro-6H-cyclopenta[e]pyrazolo[1,5-a]pyrimidine-6-carboxamide

The relative and absolute stereochemistry of each example was not determined.

### Step 1: (trans)-N-(5-cyano-6-(difluoromethoxy)pyridin-3-yl)-8-(1-(difluoromethyl)-1H-pyrazol-3-yl)-2-fluoro-8-methyl-7,8-dihydro-6H-cyclopenta[e]pyrazolo[1,5-a]pyrimidine-6-carboxamide and (cis)-N-(5-cyano-6-(difluoromethoxy)pyridin-3-yl)-8-(1-(difluoromethyl)-1H-pyrazol-3-yl)-2-fluoro-8-methyl-7,8-dihydro-6H-cyclopenta[e]pyrazolo[1,5-a]pyrimidine-6-carboxamide

In analogy to Method A1 step 8 but using 8-(1-(difluoromethyl)-1H-pyrazol-3-yl)-2-fluoro-8-methyl-7,8-dihydro-6H-cyclopenta[e]pyrazolo[1,5-a]pyrimidine-6-carboxylic acid (110 mg, 313 mol) and 5-amino-2-(difluoromethoxy)nicotinonitrile (116 mg, 626 mmol), racemic mixtures of the title compounds were obtained: trans isomer (35 mg, 21% yield) as a white solid, LC-MS (ES, m/z): 519[M+H]⁺; cis-isomer (20 mg, 10% yield) as a white solid, LC-MS (ES, m/z): 519[M+H]⁺.

### Step 2: Separation of Enantiomers to Obtain Example 110 and Example 111

35 mg of (trans)-N-(5-cyano-6-(difluoromethoxy)pyridin-3-yl)-8-(1-(difluoromethyl)-1H-pyrazol-3-yl)-2-fluoro-8-methyl-7,8-dihydro-6H-cyclopenta[e]pyrazolo[1,5-a]pyrimidine-6-carboxamide were submitted to chiral HPLC purification (Column: CHIRALPAK IF, 2*25 cm, 5 m; Mobile Phase A: Hex: DCM=3:1(0.1% FA)-HPLC, Mobile Phase B: EtOH-HPLC; Flow rate: 20 ML/MIN mL/min; Gradient: isocratic 50% Mobile Phase B; Wave Length: 254/220 nm nm; RT1 (min): 3.654; RT2 (min): 4.387; Sample Solvent: EtOH-HPLC; Injection Volume: 0.5 mL; Number Of Runs: 4). The second eluting isomer was concentrated and lyophilized to afford Example 110 (11.4 mg, 37% yield) as a white solid. The first eluting isomer was concentrated and lyophilized to afford Example 111 (14.9 mg, 47% yield) as a white solid.

Example 110: ¹H NMR (400 MHz, Chloroform-d) δ 8.59-8.62 (m, 2H), 8.54 (s, 1H), 7.85 (s, 1H), 7.75 (d, J=2.4 Hz, 1H), 7.47 (t, J=71.6 Hz, 1H), 7.05 (t, J=60.8 Hz, 1H), 6.78 (d, J=2.8 Hz, 1H), 6.31 (d, J=4.8 Hz, 1H), 4.50-4.56 (m, 1H), 3.39-3.44 (m, 1H), 2.58-2.63 (m, 1H), 2.16 (s, 3H). LC-MS (ES, m/z): 519[M+H]⁺.

Example 111: ¹H NMR (400 MHz, Chloroform-d) δ 8.61 (d, J=2.4 Hz, 1H), 8.57 (s, 1H), 8.53 (d, J=2.4 Hz, 1H), 7.83 (s, 1H), 7.75 (d, J=2.8 Hz, 1H), 7.47 (t, J=71.6 Hz, 1H), 7.05 (t, J=60.8 Hz, 1H), 6.78 (d, J=2.8 Hz, 1H), 6.30 (d, J=5.2 Hz, 1H), 4.48-4.53 (m, 1H), 3.39-3.45 (m, 1H), 2.57-2.62 (m 1H), 2.16 (s, 3H). LC-MS (ES, m/z): 519[M+H]⁺.

### Step 3: Separation of Enantiomers to Obtain Example 112 and Example 113

20 mg of (cis)-N-(5-cyano-6-(difluoromethoxy)pyridin-3-yl)-8-(1-(difluoromethyl)-11H-pyrazol-3-yl)-2-fluoro-8-methyl-7,8-dihydro-6H-cyclopenta[e]pyrazolo[1,5-a]pyrimidine-6-carboxamide were submitted to chiral HPLC purification (Column: CHIRALPAK IG, 2*25 cm, 5 m; Mobile Phase A: Hex: DCM=3:1(0.1% FA)-HPLC, Mobile Phase B: EtOH-HPLC; Flow rate: 20 ML/MIN mL/min; Gradient: isocratic 30% Mobile Phase B; Wave Length: 254/220 nm nm; RT1 (min): 4.302; RT2 (min): 5.481; Sample Solvent: EtOH-HPLC; Injection Volume: 0.6 mL; Number Of Runs: 3). The second eluting isomer was concentrated and lyophilized to afford Example 112 (6.7 mg, 33% yield) as a white solid. The first eluting isomer was concentrated and lyophilized to afford Example 113 (6.2 mg, 30% yield) as a white solid.

Example 112: ¹H NMR (400 MHz, Chloroform-d) δ 9.52 (s, 1H), 8.63 (s, 1H), 8.39-8.40 (m, 1H), 8.35-8.36 (m, 1H), 7.80 (d, J=2.8 Hz, 1H), 7.46 (t, J=71.6 Hz, 1H), 7.00 (d, J=2.8 Hz, 1H), 6.96 (t, J=60.4 Hz, 1H), 6.31 (d, J=4.8 Hz, 1H), 4.52-4.55 (m, 1H), 3.33-3.38 (m, 1H), 2.84-2.90 (m, 1H), 2.15 (s, 311). LC-MS (ES, m/z): 519[M+H]⁺.

Example 113: ¹H NMR (400 MHz, Chloroform-d) δ 9.52 (s, 1H), 8.63 (s, 1H), 8.39-8.40 (m, 1H), 8.35-8.36 (m, 1H), 7.80 (d, J=2.8 Hz, 1H), 7.46 (t, J=71.6 Hz, 1H), 7.00 (d, J=2.8 Hz, 1H), 6.96 (t, J=60.4 Hz, 1H), 6.31 (d, J=4.8 Hz, 1H), 4.52-4.55 (m, 1H), 3.33-3.38 (m, 1H), 2.84-2.90 (m, 1H), 2.15 (s, 3H). LC-MS (ES, m/z): 519[M+H]⁺.

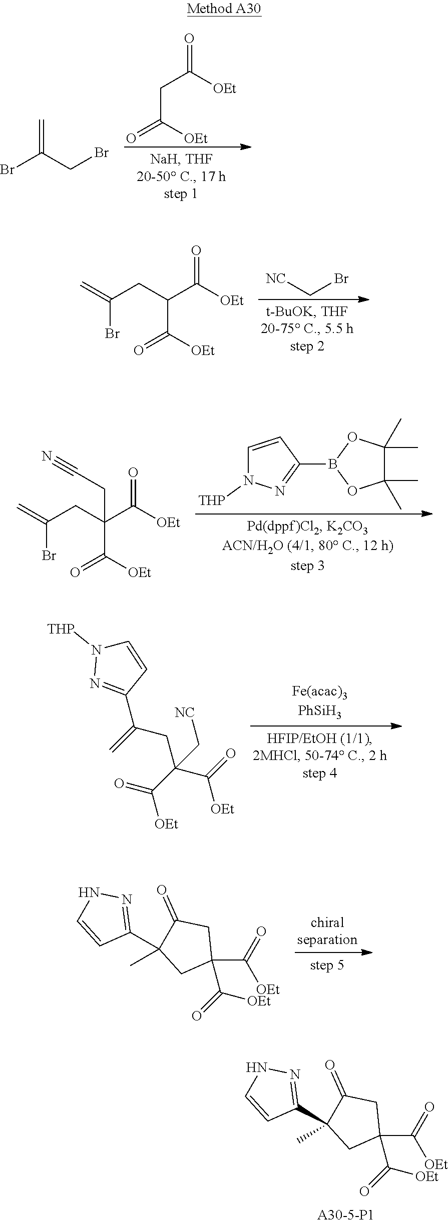

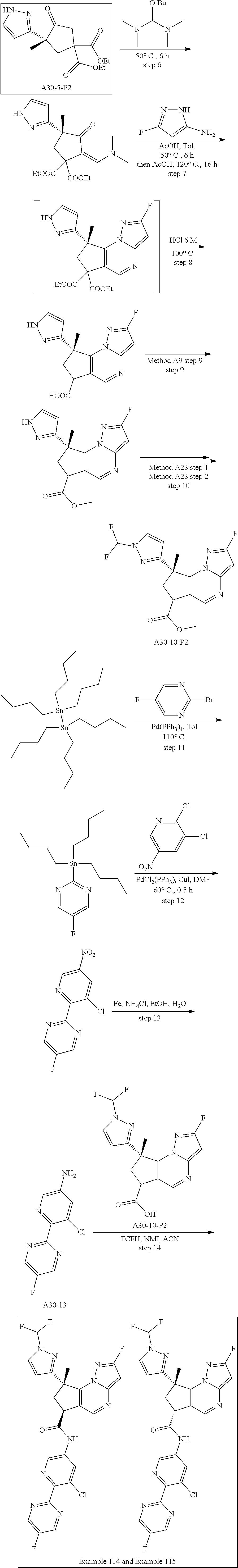

### Examples 114, 115: (6S,8S)—N-(5-chloro-6-(5-fluoropyrimidin-2-yl)pyridin-3-yl)-8-(1-(difluoromethyl)-1H-pyrazol-3-yl)-2-fluoro-8-methyl-7,8-dihydro-6H-cyclopenta[e]pyrazolo[1,5-a]pyrimidine-6-carboxamide; (6R,8S)—N-(5-chloro-6-(5-fluoropyrimidin-2-yl)pyridin-3-yl)-8-(1-(difluoromethyl)-1H-pyrazol-3-yl)-2-fluoro-8-methyl-7,8-dihydro-6H-cyclopenta[e]pyrazolo[1,5-a]pyrimidine-6-carboxamide

The relative and absolute stereochemistry of each example was not determined.

### Step 1: Diethyl 2-(2-bromoallyl)propanedioate

To a mixture of NaH (1.25 g, 32.5 mmol, 60% in mineral oil) in THF (35.0 mL) was added diethyl propanedioate (6.0 g, 37.5 mmol) at 20° C., and the mixture was stirred at 20° C. for 1 hr. The mixture was cooled to 0° C., and 2,3-dibromoprop-1-ene (5.0 g, 25.0 mmol) was added. The mixture was stirred at 50° C. for 16 h. The mixture was poured into sat. NH₄Cl (50 mL), and it was extracted with EtOAc (2×30 mL). The combined organic layers were washed with brine (20 mL), dried over anhydrous Na₂SO₄, filtered and concentrated under reduced pressure. The residue was purified by column chromatography (SiO₂, Petroleum ether/Ethyl acetate=100/1 to 10/1) to give the title compound (2.00 g, 7.17 mmol) as a colorless oil.

### Step 2: Diethyl 2-(2-bromoallyl)-2-(cyanomethyl)propanedioate

To a solution of diethyl 2-(2-bromoallyl)propanedioate (10.0 g, 35.8 mmol) in THF (70.0 mL) was added t-BuOK (4.82 g, 42.9 mmol) at 20° C. and the mixture was stirred at 20° C. for 0.5 h. 2-bromoacetonitrile (5.16 g, 42.9 mmol) was added and the mixture was stirred at 75° C. for 5 h. The mixture was poured into H₂O (100 mL), and it was extracted with EtOAc (2×50 mL). The combined organic layers were washed with brine (50 mL), dried over anhydrous Na₂SO₄, filtered and concentrated under reduced pressure. The residue was purified by column chromatography (SiO₂, Petroleum ether/Ethyl acetate=100/1 to 5/1) to give the title compound (8.90 g, 27.9 mmol) as a colorless oil.

### Step 3: Diethyl 2-(cyanomethyl)-2-[2-(1-tetrahydropyran-2-ylpyrazol-3-yl)allyl]propanedioate

To a mixture of diethyl 2-(2-bromoallyl)-2-(cyanomethyl)propanedioate (10.0 g, 31.4 mmol) and 1-tetrahydropyran-2-yl-3-(4,4,5,5-tetramethyl-1,3,2-dioxaborolan-2-yl)pyrazole (17.4 g, 62.8 mmol) in dioxane (80.0 mL) and H₂O (20.0 mL) were added Pd(dppf)C12 (2.30 g, 3.14 mmol) and K₂CO₃ (8.69 g, 62.8 mmol) under N2. The mixture was stirred at 100° C. for 16 h under N2. The mixture was poured into H₂O (100 mL), and it was extracted with EtOAc (2×100 mL).

The combined organic layers were washed with brine (100 mL), dried over anhydrous Na₂SO₄, filtered and concentrated under reduced pressure. The residue was purified by column chromatography (SiO₂, Petroleum ether/Ethyl acetate=30/1 to 1/1) to obtain the title compound (2.30 g, 5.91 mmol, 18.7% yield) as a colorless oil. LC-MS: m/z 390 [M+H]⁺.

### Step 4: Diethyl 3-methyl-4-oxo-3-(1H-pyrazol-3-yl)cyclopentane-1,1-dicarboxylate

A solution of PhSiH₃ (3.75 g, 34.6 mmol) in HFIP (22.5 mL) and EtOH (22.5 mL) was added to a flask containing diethyl 2-(cyanomethyl)-2-(2-(1-(tetrahydro-2H-pyran-2-yl)-11H-pyrazol-3-yl)allyl)malonate (4.50 g, 11.5 mmol) and Fe(acac)₃ (2.04 g, 5.78 mmol) under N2. The reaction mixture was stirred at 50° C. for 1 h under N2. HCl (2M, 45 mL) was added, and the reaction mixture was stirred at 75° C. for 1 h under N₂. The reaction mixture was poured into H₂O (10 mL), and it was extracted with EtOAc (2×30 mL). The combined organic layers were washed with brine (20 mL), dried over anhydrous Na₂SO₄ and concentrated under reduced pressure. The residue was purified by column chromatography (SiO₂, Petroleum ether/Ethyl acetate=100/1 to 0/1) to obtain a racemic mixture of the title compound (1.20 g) as a yellow oil. LC-MS: m/z 309 [M+H]⁺.

### Step 5: Separation of enantiomers to obtain diethyl (S)-3-methyl-4-oxo-3-(1H-pyrazol-3-yl)cyclopentane-1,1-dicarboxylate (A30-5-P1) and diethyl (R)-3-methyl-4-oxo-3-(1H-pyrazol-3-yl)cyclopentane-1,1-dicarboxylate (A30-5-P2)

Diethyl 3-methyl-4-oxo-3-(1H-pyrazol-3-yl)cyclopentane-1,1-dicarboxylate (1.20 g) was submitted to chiral SFC (Column: DAICEL CHIRALCEL OJ (250 mm*30 mm, 10 um); Mobile phase: A for CO₂ and B for IPA (0.1% NH3H₂O); Gradient: isocratic 25% Mobile Phase B; Flow rate: 54 g/min; Wavelength:220 nm; Column temperature: 35° C. System back pressure: 100 bar. RT1 (min) 3.7; RT2 (min)5.3. Sample Solvent: IPA-Preparative; Injection Volume: 2 mL; Number Of Runs: 40). The first eluting isomer was lyophilized to afford A30-5-P1 (430 mg) as a yellow oil, LC-MS: m/z 309 [M+H]⁺. The second eluting isomer was lyophilized to afford A30-5-P2 (440 mg) as a yellow oil, LC-MS: m/z 309 [M+H]⁺.

### Step 6: Diethyl (R)-2-((dimethylamino)methylene)-4-methyl-3-oxo-4-(1H-pyrazol-3-yl)cyclopentane-1,1-dicarboxylate

A solution of diethyl (R)-3-methyl-4-oxo-3-(1H-pyrazol-3-yl)cyclopentane-1,1-dicarboxylate (A30-5-P2, 3.80 g, 12.3 mmol) in 1-tert-butoxy-N,N,N′,N′-tetramethylmethanediamine (6.44 g, 36.9 mmol) was stirred at 50° C. for 6 h. The reaction mixture was concentrated under reduced pressure to give the crude title compound (4.60 g), which was used in the next step without further purification. LC-MS: m/z 364 [M+H]⁺.

### Step 7: Diethyl (S)-2-fluoro-8-methyl-8-(1H-pyrazol-3-yl)-7,8-dihydro-6H-cyclopenta[e]pyrazolo[1,5-a]pyrimidine-6,6-dicarboxylate

To a solution of diethyl (R)-2-((dimethylamino)methylene)-4-methyl-3-oxo-4-(11H-pyrazol-3-yl)cyclopentane-1,1-dicarboxylate (4.60 g, 12.6 mmol) in AcOH (5 mL) and toluene (50 mL) was added 3-fluoro-1H-pyrazol-5-amine (1.54 g, 15.1 mmol). The mixture was stirred at 50° C. for 6 h. Ice-water (50 mL) was added, and it was extracted with EtOAc (2×30 mL). The combined organic layers were washed with brine (50 mL), dried over anhydrous Na₂SO₄ and concentrated under reduced pressure. AcOH (50 mL) was added, and it was stirred at 120° C. for 16 h. The reaction mixture was concentrated under reduced pressure until the remaining volume of AcOH was approximately 5 mL. This crude solution of the title compound in AcOH (˜5 mL) was directly used in the next step. LC-MS: m/z 402 [M+H]⁺.

### Step 8: (8S)-2-fluoro-8-methyl-8-(1H-pyrazol-3-yl)-7,8-dihydro-6H-cyclopenta[e]pyrazolo[1,5-a]pyrimidine-6-carboxylic acid

To a solution of diethyl (S)-2-fluoro-8-methyl-8-(1H-pyrazol-3-yl)-7,8-dihydro-6H-cyclopenta[e]pyrazolo[1,5-a]pyrimidine-6,6-dicarboxylate in AcOH (˜5 mL), which was obtained from the previous step 7, was added HCl (6M, 50 mL) at 25° C. The reaction mixture was stirred at 100° C. and for 3 h. The mixture was cooled and concentrated under reduced pressure to give the crude title compound (3.30 g) as a yellow oil, which was used in the next step without further purification. LC-MS: m/z 302 [M+H]⁺.

### Step 9: Methyl (8S)-2-fluoro-8-methyl-8-(1H-pyrazol-3-yl)-7,8-dihydro-6H-cyclopenta[e]pyrazolo[1,5-a]pyrimidine-6-carboxylate

Using crude (8S)-2-fluoro-8-methyl-8-(1H-pyrazol-3-yl)-7,8-dihydro-6H-cyclopenta[e]pyrazolo[1,5-a]pyrimidine-6-carboxylic acid (3.30 g) and following the reaction conditions described in Method A9 step 9, the crude title compound (3.00 g) was obtained as a yellow oil, which was used in the next step without purification. LC-MS: m/z 316 [M+H]⁺.

### Step 10: Methyl (8S)-8-(1-(difluoromethyl)-1H-pyrazol-3-yl)-2-fluoro-8-methyl-7,8-dihydro-6H-cyclopenta[e]pyrazolo[1,5-a]pyrimidine-6-carboxylic acid (A30-10-P2)

Using methyl (8S)-2-fluoro-8-methyl-8-(1H-pyrazol-3-yl)-7,8-dihydro-6H-cyclopenta[e]pyrazolo[1,5-a]pyrimidine-6-carboxylate, and following sequentially the reaction conditions described in Method A23 step 1 and step 2, the title compound (A30-10-P2) was obtained as a yellow oil. LC-MS: m/z 352 [M+H]⁺.

### Step 11: 5-fluoro-2-(tributylstannyl)pyrimidine (A30-11)

To a stirred solution of 2-bromo-5-fluoropyrimidine (4.9 g, 27.6 mmol) in toluene (85 mL) was added 1,1,1,2,2,2-hexabutyldistannane (32 g, 55.2 mmol) and Pd(PPh₃)₄(3.2 g, 2.8 mmol). The resulting mixture was stirred at 110° C. for 24 h. The reaction was quenched with water (50 mL). The resulting mixture was extracted with ethyl acetate (3×50 mL). The combined organic layers were dried over anhydrous sodium sulfate and concentrated under reduced pressure. The residue was purified by column chromatography on silica gel using 85% petroleum ether and 15% ethyl acetate as eluent to afford the title compound (A30-11, 4.6 g, 35% yield) as a yellow solid. LC-MS: m/z 389 [M+H]⁺.

### Step 12: 2-(3-chloro-5-nitropyridin-2-yl)-5-fluoropyrimidine

To a stirred solution of 5-fluoro-2-(tributylstannyl)pyrimidine (1.7 g, 4.4 mmol) in DMF (16 mL) were added 2,3-dichloro-5-nitropyridine (932 mg, 4.8 mmol), PdCl₂(PPh₃)₂(616 mg, 878 μmol) and CuI (167 mg, 878 mol). The resulting mixture was stirred at 60° C. for 0.5 h under nitrogen atmosphere. The mixture was allowed to cool to 25° C. and quenched with water (30 mL). The resulting mixture was extracted with ethyl acetate (3×30 mL). The combined organic layers were dried over anhydrous sodium sulfate and concentrated under reduced pressure. The residue was purified by column chromatography on silica gel using 50% petroleum ether and 50% ethyl acetate as eluent to afford the title compound (340 mg, 24% yield) as a yellow solid. LC-MS: m/z 255 [M+H]⁺.

### Step 13: 5-chloro-6-(5-fluoropyrimidin-2-yl)pyridin-3-amine (A30-13)

To a stirred mixture of 2-(3-chloro-5-nitropyridin-2-yl)-5-fluoropyrimidine (300 mg, 1.2 mmol) in EtOH (5 mL) and water (1 mL) were added Fe (197 mg, 3.5 mmol) and NH₄Cl (315 mg, 5.9 mmol). The resulting mixture was stirred at 80° C. for 1 h. The solids were filtered out. The filtrate was diluted with water (20 mL) and extracted with ethyl acetate (3×20 mL). The combined organic layers were dried over anhydrous sodium sulfate and concentrated under reduced pressure.

The residue was purified by reverse phase flash chromatography to afford the title compound (A30-13, 80 mg, 28% yield) as a yellow solid. LC-MS: m/z 225 [M+H]⁺.

### Step 14: Example 114 and Example 115

In analogy to Method A1 step 8 but using (8S)-8-(1-(difluoromethyl)-1H-pyrazol-3-yl)-2-fluoro-8-methyl-7,8-dihydro-6H-cyclopenta[e]pyrazolo[1,5-a]pyrimidine-6-carboxylic acid (A30-10-P2, 41 mg, 116 μmol) and 5-chloro-6-(5-fluoropyrimidin-2-yl)pyridin-3-amine (39 mg, 174 mol), the diastereomeric title compounds were obtained: trans isomer Example 114 (2.8 mg, 4% yield) as a white solid and cis-isomer Example 115 (1.1 mg, 2% yield) as a white solid.

Example 114: ¹H NMR (400 MHz, DMSO-d₆) δ 11.01 (s, 1H), 9.08 (s, 2H), 8.80 (s, 1H), 8.72 (s, 1H), 8.47 (s, 1H), 8.21 (d, J=2.8 Hz, 1H), 7.70 (t, J=59.2 Hz, 1H), 6.63 (d, J=2.8 Hz, 1H), 6.58 (d, J=4.8 Hz, 1H), 4.48-4.54 (m, 1H), 3.00-3.07 (m, 1H), 2.49-2.60 (m, 1H), 2.04 (s, 3H). LC-MS: m/z 558 [M+H]⁺.

Example 115: ¹H NMR (400 MHz, DMSO-d₆) δ 10.93 (s, 1H), 9.08 (s, 2H), 8.78 (s, 1H), 8.73 (s, 1H), 8.42 (s, 1H), 8.15 (s, 1H), 7.71 (t, J=59.2 Hz, 1H), 6.59 (d, J=4.4 Hz, 1H), 6.48 (s, 1H), 4.55-4.59 (m, 1H), 2.82-2.87 (m, 2H), 1.92 (s, 3H). LC-MS: m/z 558 [M+H]⁺.

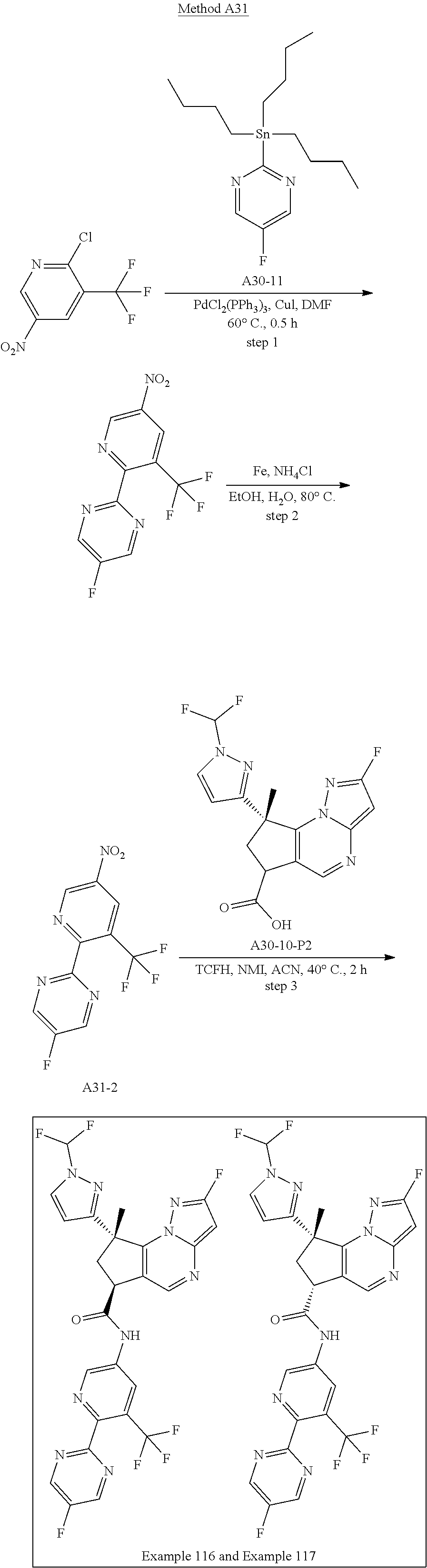

### Examples 116, 117: (6S,8S)-8-(1-(difluoromethyl)-1H-pyrazol-3-yl)-2-fluoro-N-(6-(5-fluoropyrimidin-2-yl)-5-(trifluoromethyl)pyridin-3-yl)-8-methyl-7,8-dihydro-6H-cyclopenta[e]pyrazolo[1,5-a]pyrimidine-6-carboxamide; (6R,8S)-8-(1-(difluoromethyl)-1H-pyrazol-3-yl)-2-fluoro-N-(6-(5-fluoropyrimidin-2-yl)-5-(trifluoromethyl)pyridin-3-yl)-8-methyl-7,8-dihydro-6H-cyclopenta[e]pyrazolo[1,5-a]pyrimidine-6-carboxamide

The relative and absolute stereochemistry of each example was not determined.

### Step 1: 5-fluoro-2-(5-nitro-3-(trifluoromethyl)pyridin-2-yl)pyrimidine

In analogy to Method A30 step 12 but using 2-chloro-5-nitro-3-(trifluoromethyl)pyridine (966 mg, 4.3 mmol), the title compound was obtained as a yellow solid (451 mg, 34% yield). LC-MS: m/z 289[M+H]⁺.

### Step 2: 6-(5-fluoropyrimidin-2-yl)-5-(trifluoromethyl)pyridin-3-amine (A31-2)

In analogy to Method A30 step 13 but using 5-fluoro-2-(5-nitro-3-(trifluoromethyl)pyridin-2-yl)pyrimidine (190 mg, 659 mol), the title compound was obtained as a yellow solid (A31-2, 95 mg, 53% yield). LC-MS: m/z 259 [M+H]⁺.

### Step 3: Example 116 and Example 117

In analogy to Method A1 step 8 but using (8S)-8-(1-(difluoromethyl)-1H-pyrazol-3-yl)-2-fluoro-8-methyl-7,8-dihydro-6H-cyclopenta[e]pyrazolo[1,5-a]pyrimidine-6-carboxylic acid (A30-10-P2, 40 mg, 114 μmol) and 6-(5-fluoropyrimidin-2-yl)-5-(trifluoromethyl)pyridin-3-amine (35 mg, 136 μmol), the diastereomeric title compounds were obtained: trans isomer Example 116 (7.6 mg, 11% yield) as a white solid and cis-isomer Example 117 (5.2 mg, 8% yield) as a white solid.

Example 116: ¹H NMR (400 MHz, DMSO-d₆) δ 11.14 (s, 1H), 9.08 (s, 2H), 9.07 (s, 1H), 8.75 (s, 1H), 8.72 (d, J=2.4 Hz, 1H), 8.21 (d, J=2.8 Hz, 1H), 7.70 (t, J=59.2 Hz, 1H), 6.63 (d, J=2.8 Hz, 1H), 6.59 (d, J=4.8 Hz, 1H), 4.51-4.56 (t, J=8.1 Hz, 1H), 3.00-3.08 (m, 1H), 2.57-2.63 (m, 1H), 2.04 (s, 3H). LC-MS: m/z 592 [M+H]⁺.

Example 117: ¹H NMR (400 MHz, DMSO-d₆) δ 11.05 (s, 1H), 9.08 (s, 2H), 9.05 (s, 1H), 8.75 (s, 1H), 8.67 (d, J=2.4 Hz, 1H), 8.14 (d, J=2.4 Hz, 1H), 7.69 (t, J=59.2 Hz, 1H), 6.57 (d, J=4.8 Hz, 1H), 6.49 (d, J=2.8 Hz, 1H), 4.59 (t, J=7.7 Hz, 1H), 2.90-2.95 (m, 1H), 2.81-2.86 (m, 1H), 1.93 (s, 3H). LC-MS: m/z 592 [M+H]⁺.

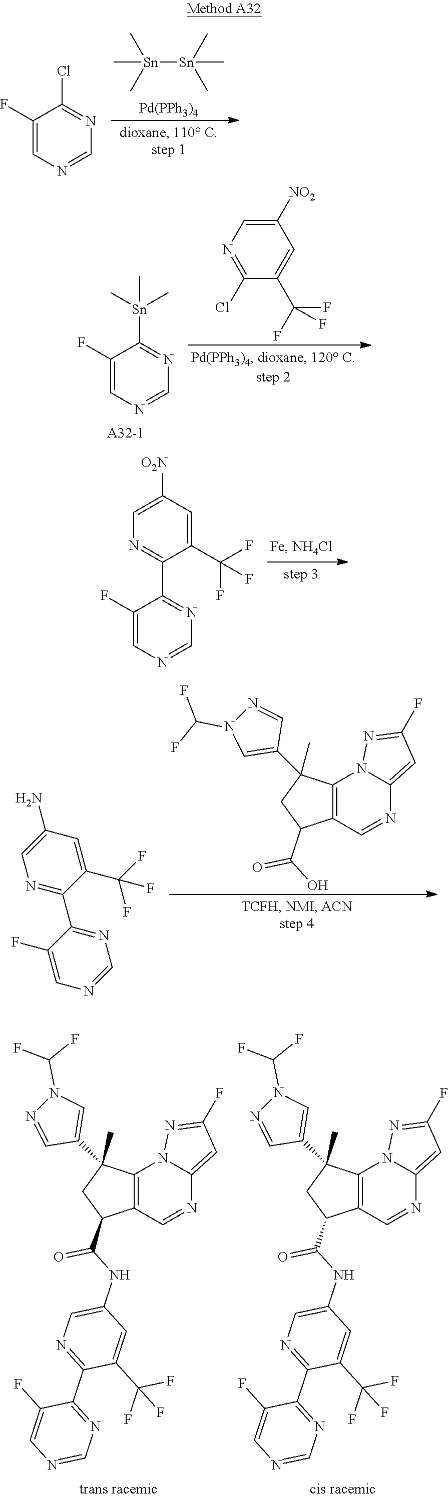

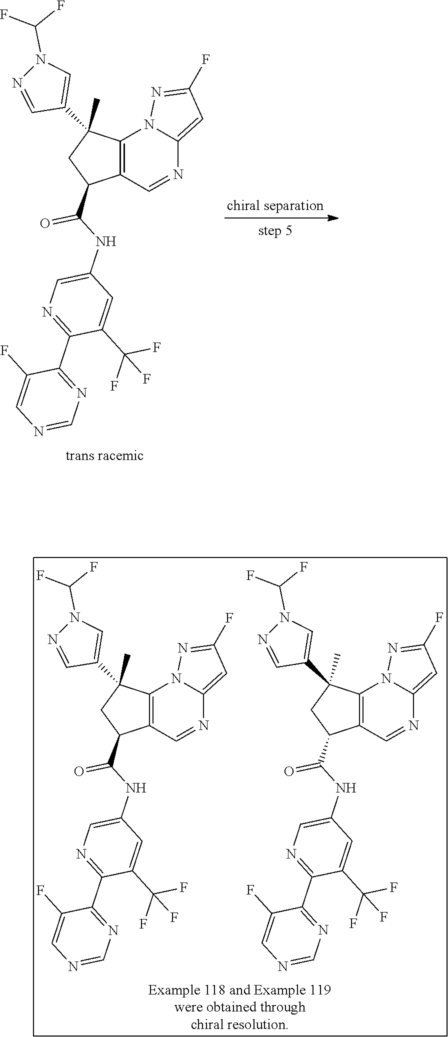

### Examples 118, 119: (6S,8R)-8-(1-(difluoromethyl)-1H-pyrazol-4-yl)-2-fluoro-N-(6-(5-fluoropyrimidin-4-yl)-5-(trifluoromethyl)pyridin-3-yl)-8-methyl-7,8-dihydro-6H-cyclopenta[e]pyrazolo[1,5-a]pyrimidine-6-carboxamide; (6R,8S)-8-(1-(difluoromethyl)-1H-pyrazol-4-yl)-2-fluoro-N-(6-(5-fluoropyrimidin-4-yl)-5-(trifluoromethyl)pyridin-3-yl)-8-methyl-7,8-dihydro-6H-cyclopenta[e]pyrazolo[1,5-a]pyrimidine-6-carboxamide

The relative and absolute stereochemistry of each example was not determined.

### Step 1: 5-fluoro-4-(trimethylstannyl)pyrimidine (A32-1)

To a stirred mixture of 4-chloro-5-fluoro-pyrimidine (1 g, 7.6 mmol) in dioxane (15 mL) were added Pd(PPh₃)₄(875 mg, 757 μmol) and 1,1,1,2,2,2-hexamethyldistannane (3.7 g, 11.3 mmol). The resulting mixture was stirred at 110° C. for 2 h under nitrogen atmosphere. The mixture was allowed to cool to 25° C. The solids were filtered out and the filtrate was concentrated under reduced pressure to afford the crude title compound (A32-1, 1 g). LC-MS: m/z 263 [M+H]⁺.

### Step 2: 5-fluoro-4-(5-nitro-3-(trifluoromethyl)pyridin-2-yl)pyrimidine

To a stirred mixture of 2-chloro-5-nitro-3-(trifluoromethyl)pyridine (2 g, 8.8 mmol) in dioxane (10 mL) were added 5-fluoro-4-(trimethylstannyl)pyrimidine (1 g, 3.8 mmol), PdCl₂(PPh₃)₂(532 mg, 758 μmol) and CuI (144 mg, 756 μmol). The resulting mixture was stirred at 110° C. for 1 h under nitrogen atmosphere. The mixture was allowed to cool down to 25° C. The solids were filtered out. The reaction was quenched with water (20 mL). The resulting mixture was extracted with ethyl acetate (3×20 mL). The combined organic layers were dried over anhydrous sodium sulfate and concentrated under reduced pressure. The residue was purified by column chromatography on silica gel using 80% petroleum ether and 20% ethyl acetate as eluent to afford the title compound (400 mg, 31% yield) as a yellow solid. LC-MS: m/z 289 [M+H]⁺.

### Step 3: 6-(5-fluoropyrimidin-4-yl)-5-(trifluoromethyl)pyridin-3-amine

In analogy to Method A30 step 13 but using 5-fluoro-4-(5-nitro-3-(trifluoromethyl)pyridin-2-yl)pyrimidine (0.2 g, 694 mol), the title compound was obtained as a yellow solid (150 mg, 77% yield). LC-MS: m/z 259 [M+H]⁺.

### Step 4: (trans)-8-(1-(difluoromethyl)-1H-pyrazol-4-yl)-2-fluoro-N-(6-(5-fluoropyrimidin-4-yl)-5-(trifluoromethyl)pyridin-3-yl)-8-methyl-7,8-dihydro-6H-cyclopenta[e]pyrazolo[1,5-a]pyrimidine-6-carboxamide and (cis)-8-(1-(difluoromethyl)-1H-pyrazol-4-yl)-2-fluoro-N-(6-(5-fluoropyrimidin-4-yl)-5-(trifluoromethyl)pyridin-3-yl)-8-methyl-7,8-dihydro-6H-cyclopenta[e]pyrazolo[1,5-a]pyrimidine-6-carboxamide

In analogy to Method A1 step 8 but using 8-(1-(difluoromethyl)-1H-pyrazol-4-yl)-2-fluoro-8-methyl-7,8-dihydro-6H-cyclopenta[e]pyrazolo[1,5-a]pyrimidine-6-carboxylic acid (A7-2, 100 mg, 285 μmol) and 6-(5-fluoropyrimidin-4-yl)-5-(trifluoromethyl)pyridin-3-amine (88 mg, 341 μmol), racemic mixtures of the title compounds were obtained: trans isomer (13.6 mg, 8% yield) as a white solid, LC-MS: m/z 592 [M+H]⁺; cis-isomer (6.9 mg, 4% yield) as a white solid, LC-MS: m/z 592 [M+H]⁺.

### Step 5: Separation of Enantiomers to Obtain Example 118 and Example 119

13 mg of (trans)-8-(1-(difluoromethyl)-1H-pyrazol-4-yl)-2-fluoro-N-(6-(5-fluoropyrimidin-4-yl)-5-(trifluoromethyl)pyridin-3-yl)-8-methyl-7,8-dihydro-6H-cyclopenta[e]pyrazolo[1,5-a]pyrimidine-6-carboxamide were submitted to chiral HPLC purification (Column: CHIRAL ART Cellulose-SB, 2*25 cm, 5 μm; Mobile Phase A: Hex (0.1% FA)-HPLC, Mobile Phase B: EtOH: DCM=1:1-HPLC; Flow rate: 20 ML/MIN mL/min; Gradient: isocratic 50% Mobile Phase B; Wave Length: 254/220 nm nm; RT1 (min): 3.421; RT2 (min): 6.738; Sample Solvent: EtOH-HPLC; Injection Volume: 1.2 mL; Number Of Runs: 1). The first eluting isomer was concentrated and lyophilized to afford Example 118 (2.7 mg, 22% yield) as a white solid. The second eluting isomer was concentrated and lyophilized to afford Example 119 (3.8 mg, 32% yield) as a white solid.

Example 118: ¹H NMR (400 MHz, DMSO-d₆) δ 11.23 (s, 1H), 9.22 (d, J=2.8 Hz, 1H), 9.16 (d, J=2.0 Hz, 1H), 9.13 (d, J=2.4 Hz, 1H), 8.78 (d, J=2.4 Hz, 1H), 8.75 (s, 1H), 8.21 (d, J=2.4 Hz, 1H), 7.71 (t, J=58.8 Hz, 1H), 6.64 (d, J=2.8 Hz, 1H), 6.59 (d, J=4.8 Hz, 1H), 4.54 (t, J=8.1 Hz, 1H), 3.04 (dd, J=12.8, 8.4 Hz, 1H), 2.60 (dd, J=12.8, 7.6 Hz, 1H), 2.04 (s, 3H). LC-MS: m/z 592 [M+H]⁺.

Example 119: ¹H NMR (400 MHz, DMSO-d₆) δ 11.23 (s, 1H), 9.22 (d, J=2.8 Hz, 1H), 9.16 (d, J=1.6 Hz, 1H), 9.13 (d, J=2.0 Hz, 1H), 8.78 (d, J=2.4 Hz, 1H), 8.75 (s, 1H), 8.21 (d, J=2.4 Hz, 1H), 7.71 (t, J=58.8 Hz, 1H), 6.64 (d, J=2.8 Hz, 1H), 6.59 (d, J=4.8 Hz, 1H), 4.54 (t, J=8.1 Hz, 1H), 3.04 (dd, J=13.0, 8.4 Hz, 1H), 2.60 (dd, J=13.0, 7.9 Hz, 1H), 2.04 (s, 3H). LC-MS: m/z 592 [M+H]⁺.

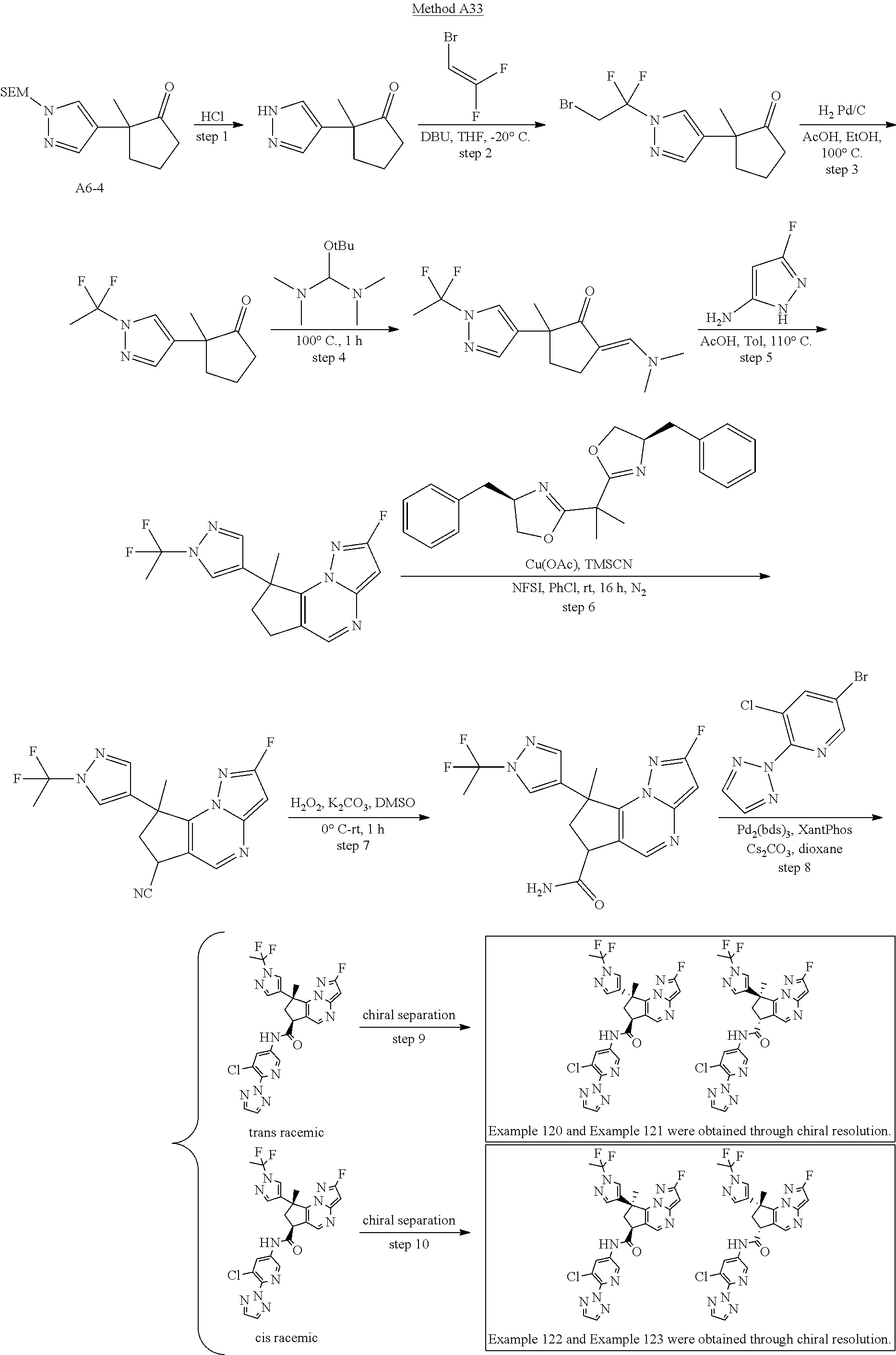

### Examples 120, 121: (6S,8R)—N-(5-chloro-6-(2H-1,2,3-triazol-2-yl)pyridin-3-yl)-8-(1-(1,1-difluoroethyl)-1H-pyrazol-4-yl)-2-fluoro-8-methyl-7,8-dihydro-6H-cyclopenta[e]pyrazolo[1,5-a]pyrimidine-6-carboxamide; (6R,8S)—N-(5-chloro-6-(2H-1,2,3-triazol-2-yl)pyridin-3-yl)-8-(1-(1,1-difluoroethyl)-1H-pyrazol-4-yl)-2-fluoro-8-methyl-7,8-dihydro-6H-cyclopenta[e]pyrazolo[1,5-a]pyrimidine-6-carboxamide

### Examples 122, 123: (6S,8S)—N-(5-chloro-6-(2H-1,2,3-triazol-2-yl)pyridin-3-yl)-8-(1-(1,1-difluoroethyl)-1H-pyrazol-4-yl)-2-fluoro-8-methyl-7,8-dihydro-6H-cyclopenta[e]pyrazolo[1,5-a]pyrimidine-6-carboxamide; (6R,8R)—N-(5-chloro-6-(2H-1,2,3-triazol-2-yl)pyridin-3-yl)-8-(1-(1,1-difluoroethyl)-1H-pyrazol-4-yl)-2-fluoro-8-methyl-7,8-dihydro-6H-cyclopenta[e]pyrazolo[1,5-a]pyrimidine-6-carboxamide

The relative and absolute stereochemistry of each example was not determined.

### Step 1: 2-methyl-2-(1H-pyrazol-4-yl)cyclopentan-1-one

A solution of 2-methyl-2-(1-((2-(trimethylsilyl)ethoxy)methyl)-1H-pyrazol-4-yl)cyclopentan-1-one (A6-4, 2 g, 6.8 mmol) in HCl (200 mL, 4M in MeOH) was stirred at 80° C. for 1 h. The mixture was cooled to room temperature, and the pH was adjusted to 7-8 with saturated aqueous sodium bicarbonate solution. The resulting mixture was extracted with ethyl acetate (3×10 mL). The combined organic layer was washed with brine, dried over anhydrous sodium sulfate, filtered and concentrated under reduced pressure to give the title compound (1 g, 6.1 mmol) as a yellow oil. LC-MS: m/z 165 [M+H]⁺.

### Step 2: 2-(1-(2-bromo-1,1-difluoroethyl)-1H-pyrazol-4-yl)-2-methylcyclopentan-1-one

To a stirred solution of 2-methyl-2-(1H-pyrazol-4-yl)cyclopentan-1-one (2 g, 12.2 mmol) and 2,3,4,6,7,8,9,10-octahydropyrimido[1,2-a]azepine (1.9 g, 12.2 mmol) in THF (200 mL) was added slowly 2-bromo-1,1-difluoroethene (1.7 g, 12.2 mmol) at −20° C. The reaction mixture was stirred at −20° C. for 3 h. The reaction was quenched with water (200 mL). The resulting mixture was extracted with ethyl acetate (3×200 mL). The combined organic layers were dried over anhydrous sodium sulfate and concentrated under reduced pressure. The residue was applied on a silica gel column and eluted with EtOAc/PE (4:1) to afford the title compound (900 mg, 24% yield) as a colorless oil. LC-MS: m/z 307 [M+H]⁺.

### Step 3: 2-(1-(1,1-difluoroethyl)-1H-pyrazol-4-yl)-2-methylcyclopentan-1-one

To a solution of 2-(1-(2-bromo-1,1-difluoroethyl)-1H-pyrazol-4-yl)-2-methylcyclopentan-1-one (900 mg, 2.9 mmol) in EtOH (20 mL) and AcOH (1 mL) was added Pd/C (125 mg, 10%). The reaction mixture was degassed and then stirred at 100° C. for 16 h under hydrogen atmosphere. The solids were filtered out and the filtrate was concentrated under reduced pressure. The residue was purified by reverse phase chromatography to afford the title compound (600 mg, 89% yield) as a yellow oil. LC-MS: m/z 229 [M+H]⁺.

### Step 4: 2-(1-(1,1-difluoroethyl)-1H-pyrazol-4-yl)-5-((dimethylamino)methylene)-2-methylcyclopentan-1-one

In analogy to Method A1 step 4 but using 2-(1-(1,1-difluoroethyl)-1H-pyrazol-4-yl)-2-methylcyclopentan-1-one (600 mg, 2.6 mmol) in 1-tert-butoxy-N,N,N′,N′-tetramethyl-methanediamine (1.4 g, 7.9 mmol) and stirring at 100° C. for 1, the crude title compound was obtained as a red oil (600 mg). LC-MS: m/z 284 [M+H]⁺.

### Step 5: 8-(1-(1,1-difluoroethyl)-1H-pyrazol-4-yl)-2-fluoro-8-methyl-7,8-dihydro-6H-cyclopenta[e]pyrazolo[1,5-a]pyrimidine

In analogy to Method A1 step 5 but using 2-(1-(1,1-difluoroethyl)-1H-pyrazol-4-yl)-5-((dimethylamino)methylene)-2-methylcyclopentan-1-one (600 mg, 2.1 mmol), the title compound was obtained as a yellow solid (600 mg, 75% yield). LC-MS: m/z 322 [M+H]⁺.

### Step 6: 8-(1-(1,1-difluoroethyl)-1H-pyrazol-4-yl)-2-fluoro-8-methyl-7,8-dihydro-6H-cyclopenta[e]pyrazolo[1,5-a]pyrimidine-6-carbonitrile

In analogy to Method A1 step 6 but using 8-(1-(1,1-difluoroethyl)-1H-pyrazol-4-yl)-2-fluoro-8-methyl-7,8-dihydro-6H-cyclopenta[e]pyrazolo[1,5-a]pyrimidine (600 mg, 1.9 mmol) in chlorobenzene (60 mL), the title compound was obtained as a yellow oil (320 mg, 44% yield). LC-MS: m/z 347 [M+H]⁺.

### Step 7: 8-(1-(1,1-difluoroethyl)-1H-pyrazol-4-yl)-2-fluoro-8-methyl-7,8-dihydro-6H-cyclopenta[e]pyrazolo[1,5-a]pyrimidine-6-carboxamide

To a stirred mixture of 8-(1-(1,1-difluoroethyl)-1H-pyrazol-4-yl)-2-fluoro-8-methyl-7,8-dihydro-6H-cyclopenta[e]pyrazolo[1,5-a]pyrimidine-6-carbonitrile (300 mg, 866 μmol) and K₂CO₃ (359 mg, 2.6 mmol) in DMSO (7 mL) was added H₂O₂(7 mL, 30% in water) dropwise at 25° C. The mixture was stirred at 25° C. for 30 min. The reaction was quenched with saturated sodium thiosulfate solution (10 mL) and diluted with water (30 mL). The resulting mixture was extracted with ethyl acetate (2×30 mL). The combined organic layers were dried over anhydrous sodium sulfate and concentrated under reduced pressure. The residue was purified by reverse phase chromatography and the collected fractions were concentrated under reduced pressure to afford the title compound (180 mg, 48% yield) as a yellow oil. LC-MS: m/z 365 [M+H]⁺.

### Step 8: (trans)-N-(5-chloro-6-(2H-1,2,3-triazol-2-yl)pyridin-3-yl)-8-(1-(1,1-difluoroethyl)-1H-pyrazol-4-yl)-2-fluoro-8-methyl-7,8-dihydro-6H-cyclopenta[e]pyrazolo[1,5-a]pyrimidine-6-carboxamide and (cis)-N-(5-chloro-6-(2H-1,2,3-triazol-2-yl)pyridin-3-yl)-8-(1-(1,1-difluoroethyl)-1H-pyrazol-4-yl)-2-fluoro-8-methyl-7,8-dihydro-6H-cyclopenta[e]pyrazolo[1,5-a]pyrimidine-6-carboxamide

To a stirred solution of 8-(1-(1,1-difluoroethyl)-1H-pyrazol-4-yl)-2-fluoro-8-methyl-7,8-dihydro-6H-cyclopenta[e]pyrazolo[1,5-a]pyrimidine-6-carboxamide (170 mg, 467 mol) in dioxane (20 mL) were added 5-bromo-3-chloro-2-(triazol-2-yl)pyridine (121 mg, 467 mol), Pd₂(dba)₃ (48 mg, 47 μmol), Xantphos (54 mg, 93 μmol) and Cs₂CO₃ (456 mg, 1.4 mmol). The mixture was stirred at 110° C. for 1 h under nitrogen atmosphere. The reaction was quenched with water (100 mL). The resulting mixture was extracted with ethyl acetate (2×100 mL). The combined organic layers were dried over anhydrous sodium sulfate and concentrated under reduced pressure. The residue was purified by reversed phase column chromatography. The obtained product was further purified by prep-achiral SFC to afford racemic mixtures of the title compounds: trans isomer (50 mg, 18% yield) as a white solid, LC-MS: m/z 543 [M+H]⁺; cis-isomer (30 mg, 11% yield) as a white solid, LC-MS: m/z 543 [M+H]⁺.

### Step 9: Separation of Enantiomers to Obtain Example 120 and Example 121

trans-N-(5-chloro-6-(2H-1,2,3-triazol-2-yl)pyridin-3-yl)-8-(1-(1,1-difluoroethyl)-1H-pyrazol-4-yl)-2-fluoro-8-methyl-7,8-dihydro-6H-cyclopenta[e]pyrazolo[1,5-a]pyrimidine-6-carboxamide (50 mg, 92 μmol) was submitted to chiral HPLC purification (Column: CHIRAL ART Cellulose-SB, 2*25 cm, 5 m; Mobile Phase A: Hex: DCM=3:1(0.1% FA)-HPLC, Mobile Phase B: EtOH-HPLC; Flow rate: 20 ML/MIN mL/min; Gradient: isocratic 50% Mobile Phase B; Wave Length: 254/220 nm nm; RT1 (min): 8.436; RT2 (min): 18.731; Sample Solvent: EtOH-HPLC; Injection Volume: 2.0 mL; Number Of Runs: 2) The first eluting isomer was concentrated and lyophilized to afford Example 120 (18.4 mg, 36% yield) as a white solid. The second eluting isomer was concentrated and lyophilized to afford Example 121 (21.1 mg, 42% yield) as a white solid.

Example 120: ¹H NMR (400 MHz, Chloroform-d) δ 8.70 (s, 1H), 8.61 (s, 1H), 8.55 (s, 1H), 8.18 (s, 1H), 7.96 (s, 2H), 7.83 (s, 1H), 7.65 (s, 1H), 6.34 (d, J=4.4 Hz, 1H), 4.40-4.45 (m, 1H), 3.01-3.06 (m, 1H), 2.73-2.78 (m, 1H), 2.26 (t, J=16.0 Hz, 3H), 2.15 (s, 3H). LC-MS: m/z 543 [M+H]⁺.

Example 121: ¹H NMR (400 MHz, Chloroform-d) δ 8.71 (s, 1H), 8.67 (s, 1H), 8.58 (s, 1H), 8.34 (s, 1H), 7.95 (s, 2H), 7.85 (s, 1H), 7.66 (s, 1H), 6.35 (d, J=4.8 Hz, 1H), 4.43-4.49 (m, 1H), 3.01-3.08 (m, 1H), 2.74-2.82 (m, 1H), 2.25 (t, J=16.0 Hz, 3H), 2.15 (s, 3H). LC-MS: m/z 543 [M+H]⁺.

### Step 10: Separation of Enantiomers to Obtain Example 122 and Example 123

cis-N-(5-chloro-6-(2H-1,2,3-triazol-2-yl)pyridin-3-yl)-8-(1-(1,1-difluoroethyl)-1H-pyrazol-4-yl)-2-fluoro-8-methyl-7,8-dihydro-6H-cyclopenta[e]pyrazolo[1,5-a]pyrimidine-6-carboxamide (30 mg, 55 mol) was submitted to chiral HPLC purification (Column: CHIRAL ART Cellulose-SB, 2*25 cm, 5 μm; Mobile Phase A: Hex: DCM=3:1(0.1% FA)-HPLC, Mobile Phase B: EtOH-HPLC; Flow rate: 20 ML/MIN mL/min; Gradient: isocratic 50% Mobile Phase B; Wave Length: 254/220 nm nm; RT1 (min): 8.855; RT2 (min): 11.409; Sample Solvent: EtOH-HPLC; Injection Volume: 1.2 mL; Number Of Runs: 2) The first eluting isomer was concentrated and lyophilized to afford Example 122 (14.3 mg, 46% yield) as a white solid. The second eluting isomer was concentrated and lyophilized to afford Example 123 (11.8 mg, 39% yield) as a white solid.

Example 122: ¹H NMR (400 MHz, Chloroform-d) δ 8.72 (s, 1H), 8.59 (s, 1H), 8.46 (s, 1H), 7.94-7.98 (m, 4H), 7.75 (s, 1H), 6.37 (d, J=5.2 Hz, 1H), 4.43-4.47 (m, 1H), 3.04-3.08 (m, 1H), 2.88-2.94 (m, 1H), 2.25 (t, J=16.0 Hz, 3H), 2.04 (s, 3H). LC-MS: m/z 543 [M+H]⁺.

Example 123: ¹H NMR (400 MHz, Chloroform-d) δ 8.68 (s, 1H), 8.59 (s, 1H), 8.44 (s, 1H), 7.90-7.96 (m, 4H), 7.75 (s, 1H), 6.35 (d, J=4.8 Hz, 1H), 4.40-4.46 (m, 1H), 3.02-3.08 (m, 1H), 2.87-2.94 (m, 1H), 2.25 (t, J=16.0 Hz, 3H), 2.03 (s, 3H). LC-MS: m/z 543 [M+H]⁺.

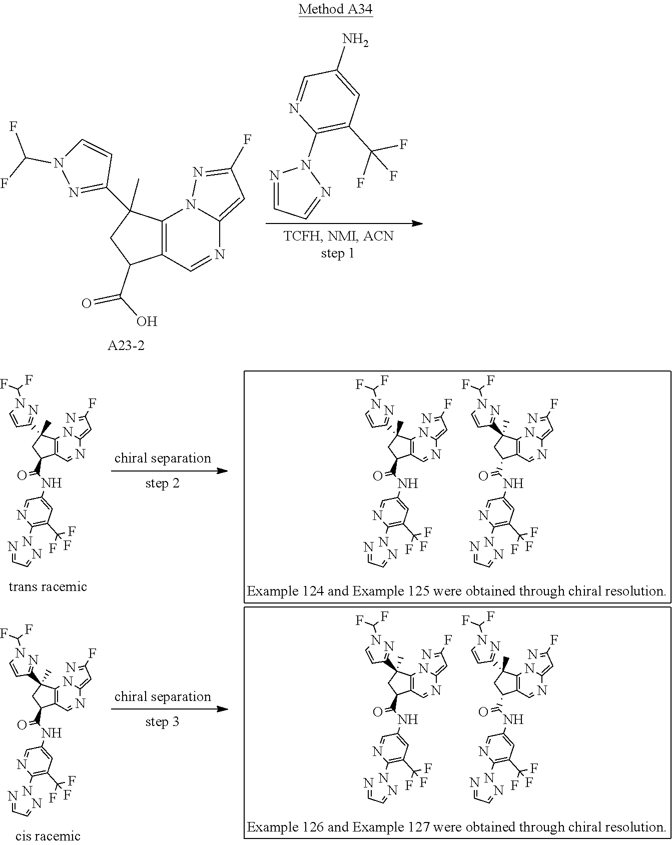

### Examples 124, 125: (6S,8S)—N-(6-(2H-1,2,3-triazol-2-yl)-5-(trifluoromethyl)pyridin-3-yl)-8-(1-(difluoromethyl)-1H-pyrazol-3-yl)-2-fluoro-8-methyl-7,8-dihydro-6H-cyclopenta[e]pyrazolo[1,5-a]pyrimidine-6-carboxamide; (6R,8R)—N-(6-(2H-1,2,3-triazol-2-yl)-5-(trifluoromethyl)pyridin-3-yl)-8-(1-(difluoromethyl)-1H-pyrazol-3-yl)-2-fluoro-8-methyl-7,8-dihydro-6H-cyclopenta[e]pyrazolo[1,5-a]pyrimidine-6-carboxamide

### Examples 126, 127: (6S,8R)—N-(6-(2H-1,2,3-triazol-2-yl)-5-(trifluoromethyl)pyridin-3-yl)-8-(1-(difluoromethyl)-1H-pyrazol-3-yl)-2-fluoro-8-methyl-7,8-dihydro-6H-cyclopenta[e]pyrazolo[1,5-a]pyrimidine-6-carboxamide; (6S,8R)—N-(6-(2H-1,2,3-triazol-2-yl)-5-(trifluoromethyl)pyridin-3-yl)-8-(1-(difluoromethyl)-1H-pyrazol-3-yl)-2-fluoro-8-methyl-7,8-dihydro-6H-cyclopenta[e]pyrazolo[1,5-a]pyrimidine-6-carboxamide

The relative and absolute stereochemistry of each example was not determined.

### Step 1: (trans)-N-(6-(2H-1,2,3-triazol-2-yl)-5-(trifluoromethyl)pyridin-3-yl)-8-(1-(difluoromethyl)-1H-pyrazol-3-yl)-2-fluoro-8-methyl-7,8-dihydro-6H-cyclopenta[e]pyrazolo[1,5-a]pyrimidine-6-carboxamide and (cis)-N-(6-(2H-1,2,3-triazol-2-yl)-5-(trifluoromethyl)pyridin-3-yl)-8-(1-(difluoromethyl)-1H-pyrazol-3-yl)-2-fluoro-8-methyl-7,8-dihydro-6H-cyclopenta[e]pyrazolo[1,5-a]pyrimidine-6-carboxamide

In analogy to Method A1 step 8 but using 8-(1-(difluoromethyl)-1H-pyrazol-3-yl)-2-fluoro-8-methyl-7,8-dihydro-6H-cyclopenta[e]pyrazolo[1,5-a]pyrimidine-6-carboxylic acid (A23-2, 70 mg, 199 μmol) and 6-(2H-1,2,3-triazol-2-yl)-5-(trifluoromethyl)pyridin-3-amine (68.5 mg, 299 mmol), racemic mixtures of the title compounds were obtained: trans isomer (30 mg, 26% yield) as a white solid, LC-MS (ES, m/z): 563[M+H]⁺; cis-isomer (20 mg, 16% yield) as a white solid, LC-MS (ES, m/z): 563[M+H]⁺.

### Step 2: Separation of Enantiomers to Obtain Example 124 and Example 125

30 mg of (trans)-N-(6-(2H-1,2,3-triazol-2-yl)-5-(trifluoromethyl)pyridin-3-yl)-8-(1-(difluoromethyl)-1H-pyrazol-3-yl)-2-fluoro-8-methyl-7,8-dihydro-6H-cyclopenta[e]pyrazolo[1,5-a]pyrimidine-6-carboxamide were submitted to chiral HPLC purification (Column: CHIRAL ART Cellulose-SB, 2*25 cm, 5 μm; Mobile Phase A: Hex: DCM=3:1(0.1% FA)-HPLC, Mobile Phase B: EtOH-HPLC; Flow rate: 20 ML/MIN mL/min; Gradient: isocratic 50% Mobile Phase B; Wave Length: 254/220 nm nm; RT1 (min): 5.954; RT2 (min): 13.761; Sample Solvent: EtOH-HPLC; Injection Volume: 2.0 mL; Number Of Runs: 2). The first eluting isomer was concentrated and lyophilized to afford Example 124 (10.0 mg, 33% yield) as a white solid. The second eluting isomer was concentrated and lyophilized to afford Example 125 (12.6 mg, 40% yield) as a white solid.

Example 124: ¹H NMR (400 MHz, DMSO-d₆) δ 11.28 (s, 1H), 9.03 (d, J=2.4 Hz, 1H), 8.83 (d, J=2.8 Hz, 1H), 8.74 (s, 1H), 8.20 (d, J=2.8 Hz, 2H), 8.19 (s, 2H), 7.70 (t, J=59.2 Hz, 1H), 6.63 (d, J=2.8 Hz, 1H), 6.59 (d, J=5.2 Hz, 1H), 4.54 (t, J=8.4 Hz, 1H), 3.02-3.07 (m, 1H), 2.57-2.62 (m, 1H), 2.04 (s, 3H). LC-MS: m/z 563 [M+H]⁺.

Example 125: ¹H NMR (400 MHz, DMSO-d₆) δ 11.28 (s, 1H), 9.03 (d, J=2.4 Hz, 1H), 8.84 (d, J=2.8 Hz, 1H), 8.74 (s, 1H), 8.20 (d, J=2.8 Hz, 3H), 8.19 (s, 2H), 7.70 (t, J=59.2 Hz, 1H), 6.63 (d, J=2.8 Hz, 1H), 6.59 (d, J=5.2 Hz, 1H), 4.54 (t, J=8.4 Hz, 1H), 3.02-3.07 (m, 1H), 2.57-2.62 (m, 1H), 2.04 (s, 3H). LC-MS: m/z 563 [M+H]⁺.

### Step 3: Separation of Enantiomers to Obtain Example 126 and Example 127

20 mg of (cis)-N-(6-(2H-1,2,3-triazol-2-yl)-5-(trifluoromethyl)pyridin-3-yl)-8-(1-(difluoromethyl)-1H-pyrazol-3-yl)-2-fluoro-8-methyl-7,8-dihydro-6H-cyclopenta[e]pyrazolo[1,5-a]pyrimidine-6-carboxamide were submitted to chiral HPLC purification (Column: CHIRAL ART Cellulose-SB, 2*25 cm, 5 μm; Mobile Phase A: Hex: DCM=3:1(0.1% FA)-HPLC, Mobile Phase B: MeOH-HPLC; Flow rate: 20 ML/MIN mL/min; Gradient: isocratic 20% Mobile Phase B; Wave Length: 254/220 nm nm; RT1 (min): 7.056; RT2 (min): 8.952; Sample Solvent: EtOH-HPLC; Injection Volume: 1.0 mL; Number Of Runs: 3). The second eluting isomer was concentrated and lyophilized to afford Example 126 (4.7 mg, 23% yield) as a white solid. The first eluting isomer was concentrated and lyophilized to afford Example 127 (6.2 mg, 30% yield) as a white solid.

Example 126: ¹H NMR (400 MHz, DMSO-d₆) δ 11.18 (s, 1H), 9.00 (d, J=2.4 Hz, 1H), 8.79 (d, J=2.4 Hz, 1H), 8.74 (s, 1H), 8.19 (s, 2H), 8.14 (d, J=2.8 Hz, 1H), 7.68 (t, J=59.2 Hz, 1H), 6.57 (d, J=4.8 Hz, 1H), 6.50 (d, J=2.8 Hz, 1H), 4.58-4.62 (m, 1H), 2.91-2.96 (m, 1H), 2.80-2.86 (m, 1H), 1.92 (s, 3H). LC-MS: m/z 563 [M+H]⁺.

Example 127: ¹H NMR (400 MHz, DMSO-d₆) δ 11.18 (s, 1H), 9.00 (d, J=2.4 Hz, 1H), 8.79 (d, J=2.4 Hz, 1H), 8.74 (s, 1H), 8.19 (s, 2H), 8.14 (d, J=2.8 Hz, 1H), 7.68 (t, J=59.2 Hz, 1H), 6.57 (d, J=4.8 Hz, 1H), 6.50 (d, J=2.8 Hz, 1H), 4.58-4.62 (m, 1H), 2.91-2.96 (m, 1H), 2.80-2.86 (m, 1H), 1.92 (s, 3H). LC-MS: m/z 563 [M+H]⁺.

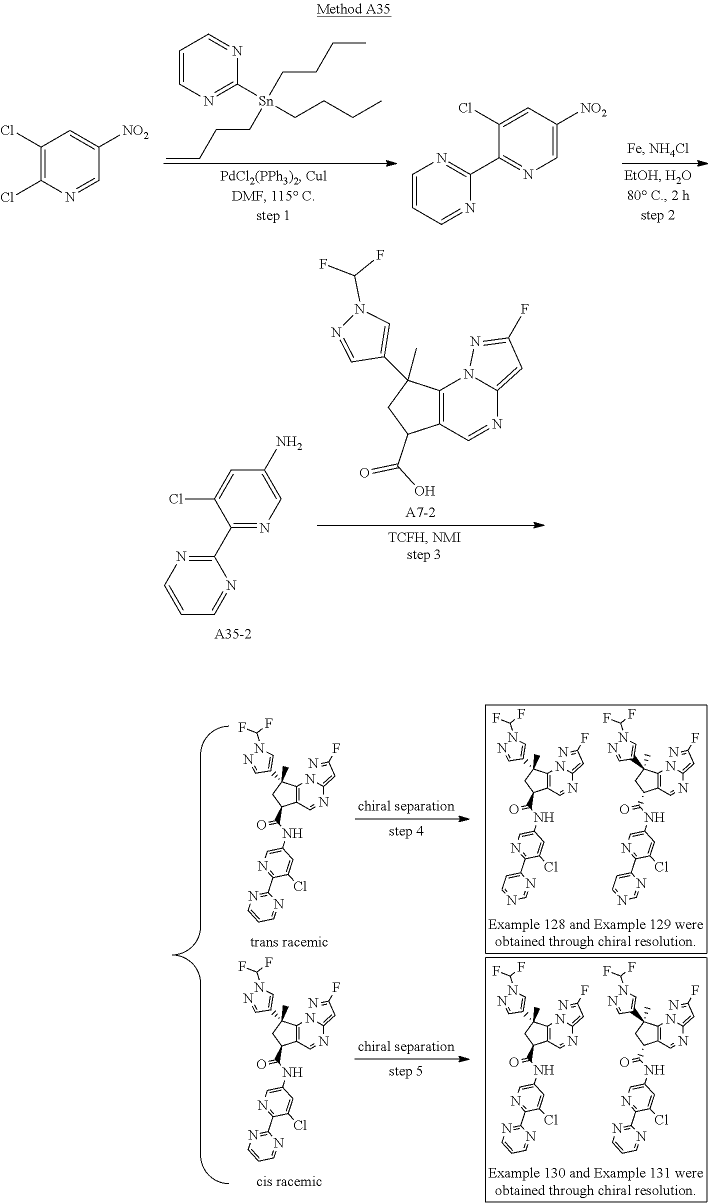

### Examples 128, 129: (6S,8R)—N-(5-chloro-6-(pyrimidin-2-yl)pyridin-3-yl)-8-(1-(difluoromethyl)-1H-pyrazol-4-yl)-2-fluoro-8-methyl-7,8-dihydro-6H-cyclopenta[e]pyrazolo[1,5-a]pyrimidine-6-carboxamide; (6R,8S)—N-(5-chloro-6-(pyrimidin-2-yl)pyridin-3-yl)-8-(1-(difluoromethyl)-1H-pyrazol-4-yl)-2-fluoro-8-methyl-7,8-dihydro-6H-cyclopenta[e]pyrazolo[1,5-a]pyrimidine-6-carboxamide

### Examples 130, 131: (6S,8S)—N-(5-chloro-6-(pyrimidin-2-yl)pyridin-3-yl)-8-(1-(difluoromethyl)-1H-pyrazol-4-yl)-2-fluoro-8-methyl-7,8-dihydro-6H-cyclopenta[e]pyrazolo[1,5-a]pyrimidine-6-carboxamide; (6R,8R)—N-(5-chloro-6-(pyrimidin-2-yl)pyridin-3-yl)-8-(1-(difluoromethyl)-1H-pyrazol-4-yl)-2-fluoro-8-methyl-7,8-dihydro-6H-cyclopenta[e]pyrazolo[1,5-a]pyrimidine-6-carboxamide

The relative and absolute stereochemistry of each example was not determined.

### Step 1: 2-(3-chloro-5-nitro-2-pyridyl)pyrimidine

To a stirred mixture of 2,3-dichloro-5-nitro-pyridine (600 mg, 3.1 mmol) and tributyl(pyrimidin-2-yl)stannane (1.3 g, 3.4 mmol) in DMF (10 mL) was added iodocopper (118 mg, 622 μmol) and bis(triphenylphosphine)palladium(H) chloride (436 mg, 622 mol). The resulting mixture was stirred at 120° C. for 16 h under nitrogen atmosphere. The reaction mixture was concentrated under reduced pressure. The residue was purified by reverse phase chromatography to afford the title compound (410 mg, 55% yield) as a yellow solid. LC-MS: m/z 237 [M+H]⁺.

### Step 2: 5-chloro-6-pyrimidin-2-yl-pyridin-3-amine (A35-2)

In analogy to Method A30 step 13 but using 2-(3-chloro-5-nitro-2-pyridyl)pyrimidine (390 mg, 1.6 mmol), the title compound was obtained as a yellow solid (A35-2, 100 mg, 29% yield) as a yellow solid. LC-MS: m/z 207 [M+H]⁺.

### Step 3: trans-N-(5-chloro-6-(pyrimidin-2-yl)pyridin-3-yl)-8-(1-(difluoromethyl)-1H-pyrazol-4-yl)-2-fluoro-8-methyl-7,8-dihydro-6H-cyclopenta[e]pyrazolo[1,5-a]pyrimidine-6-carboxamide and cis-N-(5-chloro-6-(pyrimidin-2-yl)pyridin-3-yl)-8-(1-(difluoromethyl)-1H-pyrazol-4-yl)-2-fluoro-8-methyl-7,8-dihydro-6H-cyclopenta[e]pyrazolo[1,5-a]pyrimidine-6-carboxamide

In analogy to Method A1 step 8 but using 5-chloro-6-pyrimidin-2-yl-pyridin-3-amine (A35-2, 80 mg, 387 mol) and 8-(1-(difluoromethyl)-1H-pyrazol-4-yl)-2-fluoro-8-methyl-7,8-dihydro-6H-cyclopenta[e]pyrazolo[1,5-a]pyrimidine-6-carboxylic acid (A7-2, 136 mg, 387 mol), racemic mixtures of the title compounds were obtained: trans isomer (20 mg, 16% yield) as a white solid, LC-MS: m/z 540 [M+H]⁺; cis-isomer (20 mg, 16% yield), LC-MS: m/z 540 [M+H]⁺.

### Step 4: Separation of Enantiomers to Obtain Example 128 and Example 129

Trans-N-(5-chloro-6-(pyrimidin-2-yl)pyridin-3-yl)-8-(1-(difluoromethyl)-1H-pyrazol-4-yl)-2-fluoro-8-methyl-7,8-dihydro-6H-cyclopenta[e]pyrazolo[1,5-a]pyrimidine-6-carboxamide (20 mg, 37 μmol) was submitted to chiral HPLC purification (Column: CHIRALPAK ID, 2*25 cm, 5 μm; Mobile Phase A: Hex: DCM=3:1(0.1% FA)-HPLC, Mobile Phase B: EtOH-HPLC; Flow rate: 20 ML/MIN mL/min; Gradient: isocratic 50% Mobile Phase B; Wave Length: 254/220 nm nm; RT1 (min): 5.83; RT2 (min): 9.43; Sample Solvent: EtOH-HPLC; Injection Volume: 1.5 mL; Number Of Runs: 1). The second eluting isomer was concentrated and lyophilized to afford Example 128 (6.0 mg, 29% yield) as a white solid. The first eluting isomer was concentrated and lyophilized to afford Example 129 (4.3 mg, 21% yield) as a white solid.

Example 128: ¹H NMR (400 MHz, DMSO-d₆) δ 10.96 (s, 1H), 8.98 (d, J=5.2 Hz, 2H), 8.79 (d, J=2.0 Hz, 1H), 8.71 (s, 1H), 8.46 (d, J=2.4 Hz, 1H), 8.21 (s, 1H), 7.84 (s, 1H), 7.73 (t, J=58.8 Hz, 1H), 7.62 (t, J=5.2 Hz, 1H), 6.60 (d, J=4.8 Hz, 1H), 4.56 (t, J=8.4 Hz, 1H), 2.92-2.98 (m, 1H), 2.55-2.60 (m, 1H), 2.00 (s, 3H). LC-MS: m/z 540 [M+H]⁺.

Example 129: ¹H NMR (400 MHz, DMSO-d₆) δ 10.94 (s, 1H), 8.98 (d, J=5.2 Hz, 2H), 8.79 (d, J=2.0 Hz, 1H), 8.71 (s, 1H), 8.46 (d, J=2.0 Hz, 1H), 8.21 (s, 1H), 7.84 (s, 1H), 7.73 (t, J=58.8 Hz, 1H), 7.62 (t, J=4.8 Hz, 1H), 6.60 (d, J=4.8 Hz, 1H), 4.55 (t, J=8.4 Hz, 1H), 2.92-2.98 (m, 1H), 2.55-2.60 (m, 1H), 2.00 (s, 3H). LC-MS: m/z 540 [M+H]⁺.

### Step 5: Separation of Enantiomers to Obtain Example 130 and Example 131

Cis-N-(5-chloro-6-(pyrimidin-2-yl)pyridin-3-yl)-8-(1-(difluoromethyl)-1H-pyrazol-4-yl)-2-fluoro-8-methyl-7,8-dihydro-6H-cyclopenta[e]pyrazolo[1,5-a]pyrimidine-6-carboxamide (20 mg, 37 μmol) was submitted to chiral HPLC purification (Column: CHIRAL ART Cellulose-SC, 2*25 cm, 5 m; Mobile Phase A: Hex: DCM=3:1(0.1% FA)-HPLC, Mobile Phase B: EtOH-HPLC; Flow rate: 20 ML/MIN mL/min; Gradient: isocratic 50% Mobile Phase B; Wave Length: 254/220 nm nm; RT1 (min): 9.18; RT2 (min): 19.56; Sample Solvent: EtOH-HPLC; Injection Volume: 1.8 mL; Number Of Runs: 1). The first eluting isomer was concentrated and lyophilized to afford Example 130 (2.0 mg, 10% yield) as a white solid. The second eluting isomer was concentrated and lyophilized to afford Example 131 (2.0 mg, 10% yield) as a white solid.

Example 130: ¹H NMR (400 MHz, DMSO-d₆) δ 10.98 (s, 1H), 8.98 (d, J=5.2 Hz, 2H), 8.78 (d, J=2.0 Hz, 1H), 8.75 (s, 1H), 8.42 (d, J=2.0 Hz, 1H), 8.19 (s, 1H), 7.77 (s, 1H), 7.74 (t, J=58.8 Hz, 1H), 7.61 (t, J=4.8 Hz, 1H), 6.58 (d, J=4.8 Hz, 1H), 4.53-4.58 (m, 1H), 2.82-2.90 (m, 1H), 2.71-2.78 (m, 1H), 1.91 (s, 3H). LC-MS: m/z 540 [M+H]⁺.

Example 131: ¹H NMR (400 MHz, DMSO-d₆) δ 10.94 (s, 1H), 8.98 (d, J=5.2 Hz, 2H), 8.78 (d, J=2.0 Hz, 1H), 8.75 (s, 1H), 8.42 (d, J=2.0 Hz, 1H), 8.19 (s, 1H), 7.77 (s, 1H), 7.74 (t, J=58.8 Hz, 1H), 7.60 (t, J=4.8 Hz, 1H), 6.58 (d, J=4.8 Hz, 1H), 4.53-4.58 (m, 1H), 2.82-2.90 (m, 1H), 2.71-2.78 (m, 1H), 1.91 (s, 3H). LC-MS: m/z 540 [M+H]⁺.

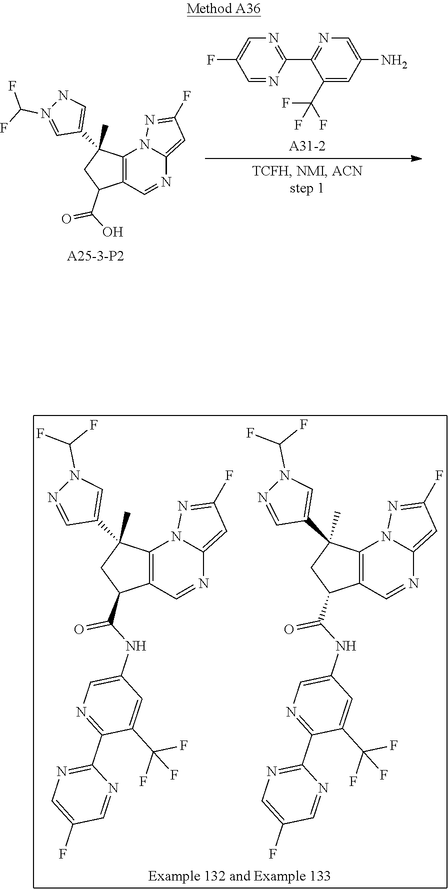

### Examples 132, 133: (6S,8R)-8-(1-(difluoromethyl)-1H-pyrazol-4-yl)-2-fluoro-N-(6-(5-fluoropyrimidin-2-yl)-5-(trifluoromethyl)pyridin-3-yl)-8-methyl-7,8-dihydro-6H-cyclopenta[e]pyrazolo[1,5-a]pyrimidine-6-carboxamide; (6R,8R)-8-(1-(difluoromethyl)-1H-pyrazol-4-yl)-2-fluoro-N-(6-(5-fluoropyrimidin-2-yl)-5-(trifluoromethyl)pyridin-3-yl)-8-methyl-7,8-dihydro-6H-cyclopenta[e]pyrazolo[1,5-a]pyrimidine-6-carboxamide

The relative and absolute stereochemistry of each example was not determined.

### Step 1: Example 132 and Example 133

In analogy to Method A1 step 8 but using 6-(5-fluoropyrimidin-2-yl)-5-(trifluoromethyl)pyridin-3-amine (A31-2, 80 mg, 310 mol) and (8R)-8-(1-(difluoromethyl)-1H-pyrazol-4-yl)-2-fluoro-8-methyl-7,8-dihydro-6H-cyclopenta[c]pyrazolo[1,5-a]pyrimidine-6-carboxylic acid (A25-3-P2, 91 mg, 259 μmol), the diastereomeric title compounds were obtained: trans isomer Example 132 (11.5 mg, 7% yield) as a white solid and cis-isomer Example 133 (15.7 mg, 10% yield) as a white solid.

Example 132: ¹H NMR (400 MHz, DMSO-d₆) δ 11.11 (s, 1H), 9.08 (s, 3H), 8.71-8.73 (m, 2H), 8.21 (s, 1H), 7.84 (s, 1H), 7.73 (t, J=58.8 Hz, 1H), 6.59 (d, J=5.2 Hz, 1H), 4.58 (t, J=8.2 Hz, 1H), 2.92-2.98 (m, 1H), 2.58-2.63 (m, 1H), 2.01 (s, 3H). LC-MS: m/z 592 [M+H]⁺.

Example 133: ¹H NMR (400 MHz, DMSO-d₆) δ 11.10 (s, 1H), 9.08 (s, 3H), 8.77 (s, 1H), 8.66 (s, 1H), 8.18 (s, 1H), 7.78 (s, 1H), 7.73 (t, J=58.8 Hz, 1H), 6.59 (d, J=4.8 Hz, 1H), 4.58 (t, J=8.2 Hz, 1H), 2.84-2.90 (m, 1H), 2.75-2.80 (m, 1H), 1.92 (s, 3H). LC-MS: m/z 592 [M+H]⁺.

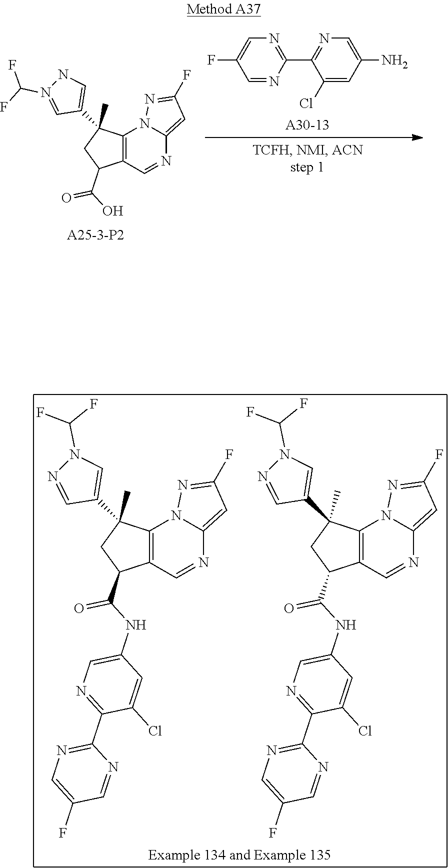

### Examples 134, 135: (6S,8R)—N-(5-chloro-6-(5-fluoropyrimidin-2-yl)pyridin-3-yl)-8-(1-(difluoromethyl)-1H-pyrazol-4-yl)-2-fluoro-8-methyl-7,8-dihydro-6H-cyclopenta[e]pyrazolo[1,5-a]pyrimidine-6-carboxamide, (6R,8R)—N-(5-chloro-6-(5-fluoropyrimidin-2-yl)pyridin-3-yl)-8-(1-(difluoromethyl)-1H-pyrazol-4-yl)-2-fluoro-8-methyl-7,8-dihydro-6H-cyclopenta[e]pyrazolo[1,5-a]pyrimidine-6-carboxamide

The relative and absolute stereochemistry of each example was not determined.

### Step 1: Example 134 and Example 135

In analogy to Method A1 step 8 but using 5-chloro-6-(5-fluoropyrimidin-2-yl)pyridin-3-amine (A30-13, 90 mg, 401 μmol) and (8R)-8-(1-(difluoromethyl)-1H-pyrazol-4-yl)-2-fluoro-8-methyl-7,8-dihydro-6H-cyclopenta[e]pyrazolo[1,5-a]pyrimidine-6-carboxylic acid (A25-3-P2, 94 mg, 267 mol), the diastereomeric title compounds were obtained: trans isomer Example 134 (5.9 mg, 4% yield) as an off-white solid and cis-isomer Example 135 (4.8 mg, 3% yield) as an off-white solid.

Example 134: ¹H NMR (400 MHz, DMSO-d₆) 10.98 (s, 1H), 9.08 (s, 2H), 8.79 (d, J=2.4 Hz, 1H), 8.75 (s, 1H), 8.43 (d, J=2.4 Hz, 1H), 8.19 (s, 1H), 7.77 (s, 1H), 7.74 (t, J=59.2 Hz, 1H), 6.58 (d, J=4.8 Hz, 1H), 4.57 (t, J=8.2 Hz, 1H), 2.83-2.89 (m, 1H), 2.72-2.78 (m, 1H), 1.91 (s, 3H). LC-MS: m/z558 [M+H]⁺.

Example 135: ¹H NMR (400 MHz, DMSO-d₆) δ 11.05 (s, 1H), 9.08 (s, 2H), 8.80 (d, J=2.0 Hz, 1H), 8.70 (s, 1H), 8.48 (d, J=2.4 Hz, 1H), 8.22 (s, 1H), 7.84 (s, 1H), 7.73 (t, J=59.2 Hz, 1H), 6.59 (d, J=4.8 Hz, 1H), 4.57 (t, J=8.2 Hz, 1H), 2.91-2.98 (m, 1H), 2.55-2.61 (m, 1H), 2.00 (s, 3H). LC-MS: m/z 558 [M+H]⁺.

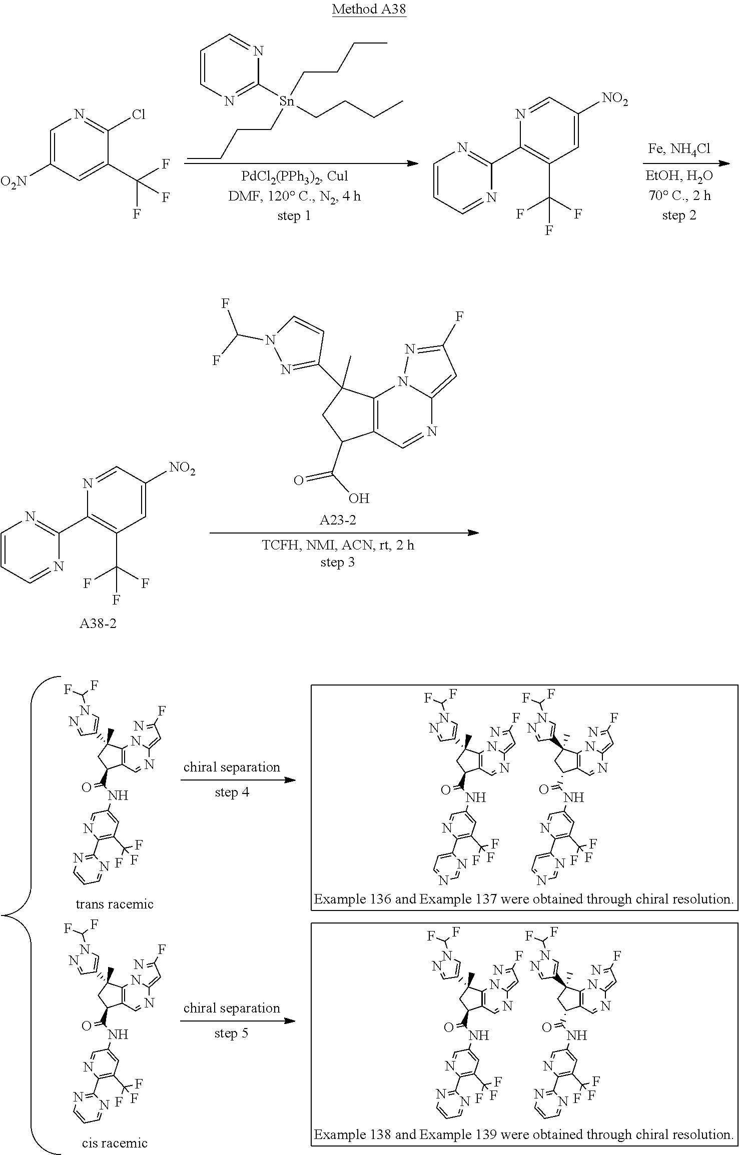

### Examples 136, 137: (6S,8S)-8-(1-(difluoromethyl)-1H-pyrazol-3-yl)-2-fluoro-8-methyl-N-(6-(pyrimidin-2-yl)-5-(trifluoromethyl)pyridin-3-yl)-7,8-dihydro-6H-cyclopenta[e]pyrazolo[1,5-a]pyrimidine-6-carboxamide, (6R,8R)-8-(1-(difluoromethyl)-1H-pyrazol-3-yl)-2-fluoro-8-methyl-N-(6-(pyrimidin-2-yl)-5-(trifluoromethyl)pyridin-3-yl)-7,8-dihydro-6H-cyclopenta[e]pyrazolo[1,5-a]pyrimidine-6-carboxamide

### Examples 138, 139: (6S,8R)-8-(1-(difluoromethyl)-1H-pyrazol-3-yl)-2-fluoro-8-methyl-N-(6-(pyrimidin-2-yl)-5-(trifluoromethyl)pyridin-3-yl)-7,8-dihydro-6H-cyclopenta[e]pyrazolo[1,5-a]pyrimidine-6-carboxamide, (6R,8S)-8-(1-(difluoromethyl)-1H-pyrazol-3-yl)-2-fluoro-8-methyl-N-(6-(pyrimidin-2-yl)-5-(trifluoromethyl)pyridin-3-yl)-7,8-dihydro-6H-cyclopenta[e]pyrazolo[1,5-a]pyrimidine-6-carboxamide

The relative and absolute stereochemistry of each example was not determined.

### Step 1: 2-(5-nitro-3-(trifluoromethyl)pyridin-2-yl)pyrimidine

In analogy to Method A35 step 1 but using 2-chloro-5-nitro-3-(trifluoromethyl)pyridine (1.2 g, 5.2 mmol) and stirring at 120° C. for 3 h, the title compound was obtained as a yellow solid (740 mg, 47% yield). LC-MS (ES, m/z): 271 [M+H]⁺.

### Step 2: 6-(pyrimidin-2-yl)-5-(trifluoromethyl)pyridin-3-amine (A38-2)

To a stirred solution of a mixture of 2-(5-nitro-3-(trifluoromethyl)pyridin-2-yl)pyrimidine (900 mg, 3.3 mmol) in EtOH (45 mL) and water (45 mL) was added Fe (744 mg, 13.3 mmol) and NH₄Cl (713 mg, 13.3 mmol). The reaction mixture was stirred at 70° C. for 2 h. The reaction mixture was filtered and the filtrate was concentrated under reduced pressure. The residue was purified by prep-HPLC to afford the title compound (A38-2, 580 mg, 71% yield) as a yellow solid. LC-MS: m/z 241 [M+H]⁺.

### Step 3: (trans)-8-(1-(difluoromethyl)-1H-pyrazol-3-yl)-2-fluoro-8-methyl-N-(6-(pyrimidin-2-yl)-5-(trifluoromethyl)pyridin-3-yl)-7,8-dihydro-6H-cyclopenta[e]pyrazolo[1,5-a]pyrimidine-6-carboxamide and (cis)-8-(1-(difluoromethyl)-1H-pyrazol-3-yl)-2-fluoro-8-methyl-N-(6-(pyrimidin-2-yl)-5-(trifluoromethyl)pyridin-3-yl)-7,8-dihydro-6H-cyclopenta[e]pyrazolo[1,5-a]pyrimidine-6-carboxamide

In analogy to Method A1 step 8 but using 8-(1-(difluoromethyl)-1H-pyrazol-3-yl)-2-fluoro-8-methyl-7,8-dihydro-6H-cyclopenta[e]pyrazolo[1,5-a]pyrimidine-6-carboxylic acid (A23-2, 150 mg, 427 mol) and 6-(pyrimidin-2-yl)-5-(trifluoromethyl)pyridin-3-amine (A38-2, 123 mg, 512 mmol), racemic mixtures of the title compounds were obtained: trans isomer (30 mg, 11% yield) as a white solid, LC-MS (ES, m/z): 574[M+H]⁺; cis-isomer (20 mg, 7% yield) as a white solid, LC-MS (ES, m/z): 574[M+H]⁺.

### Step 4: Separation of Enantiomers to Obtain Example 136 and Example 137

30 mg of (trans)-8-(1-(difluoromethyl)-1H-pyrazol-3-yl)-2-fluoro-8-methyl-N-(6-(pyrimidin-2-yl)-5-(trifluoromethyl)pyridin-3-yl)-7,8-dihydro-6H-cyclopenta[e]pyrazolo[1,5-a]pyrimidine-6-carboxamide were submitted to chiral HPLC purification (Column: CHIRALPAK ID, 2*25 cm, 5 m; Mobile Phase A: Hex (0.1% FA)-HPLC, Mobile Phase B: EtOH: DCM=1:1-HPLC; Flow rate: 20 ML/MIN mL/min; Gradient: isocratic 70% Mobile Phase B; Wave Length: 254/220 nm nm; RT1 (min): 4.369; RT2 (min): 6.209; Sample Solvent: EtOH-HPLC; Injection Volume: 0.9 mL; Number Of Runs: 3). The second eluting isomer was concentrated and lyophilized to afford Example 136 (10.2 mg, 32% yield) as a white solid. The first eluting isomer was concentrated and lyophilized to afford Example 137 (13.3 mg, 42% yield) as a white solid.

Example 136: ¹H NMR (400 MHz, Chloroform-d) δ 9.20 (s, 1H), 9.05 (s, 1H), 8.96 (d, J=4.8 Hz, 2H), 8.65 (s, 1H), 7.74 (d, J=2.4 Hz, 1H), 7.44 (t, J=4.8 Hz, 1H), 7.06 (t, J=60.8 Hz, 1H), 6.75 (d, J=2.4 Hz, 1H), 6.29 (d, J=5.2 Hz, 1H), 4.64 (t, J=7.2 Hz, 1H), 3.41-3.46 (m, 1H), 2.64-2.69 (m, 1H), 2.17 (s, 3H). LC-MS (ES, m/z): 574[M+H]⁺.

Example 137: ¹H NMR (400 MHz, Chloroform-d) δ 9.00 (s, 1H), 8.95 (d, J=5.2 Hz, 2H), 8.85 (s, 1H), 8.59 (s, 1H), 8.26 (s, 1H), 7.75 (d, J=2.4 Hz, 1H), 7.43 (t, J=4.8 Hz, 1H), 7.06 (t, J=60.8 Hz, 1H), 6.78 (d, J=2.4 Hz, 1H), 6.29 (d, J=5.2 Hz, 1H), 4.56 (t, J=8.4 Hz, 1H), 3.42-3.47 (m, 1H), 2.60-2.66 (m, 1H), 2.16 (s, 3H). LC-MS (ES, m/z): 574[M+H]⁺.

### Step 5: Separation of Enantiomers to Obtain Example 138 and Example 139

20 mg of (cis)-8-(1-(difluoromethyl)-1H-pyrazol-3-yl)-2-fluoro-8-methyl-N-(6-(pyrimidin-2-yl)-5-(trifluoromethyl)pyridin-3-yl)-7,8-dihydro-6H-cyclopenta[e]pyrazolo[1,5-a]pyrimidine-6-carboxamide were submitted to chiral HPLC purification (Column: CHIRAL ART Cellulose-SC, 2*25 cm, 5 m; Mobile Phase A: Hex (0.1% FA)-HPLC, Mobile Phase B: EtOH: DCM=1:1-HPLC; Flow rate: 20 ML/MIN mL/min; Gradient: isocratic 40% Mobile Phase B; Wave Length: 254/220 nm nm; RT1 (min): 11.491; RT2 (min): 16.05; Sample Solvent: EtOH-HPLC; Injection Volume: 0.8 mL; Number Of Runs: 3). The first eluting isomer was concentrated and lyophilized to afford Example 138 (5.5 mg, 27% yield) as a white solid. The second eluting isomer was concentrated and lyophilized to afford Example 139 (4.7 mg, 23% yield) as a white solid.

Example 138: ¹H NMR (400 MHz, Chloroform-d) δ 9.62 (s, 1H), 9.01 (s, 1H), 8.94 (d, J=4.8 Hz, 2H), 8.68 (s, 1H), 8.61 (s, 1H), 7.81 (d, J=2.4 Hz, 1H), 7.41 (t, J=4.8 Hz, 1H), 6.99 (t, J=60.8 Hz, 1H), 6.96 (d, J=2.8 Hz, 1H), 6.31 (d, J=5.2 Hz, 1H), 4.58-4.61 (m, 1H), 3.38-3.43 (m, 1H), 2.87-2.94 (m, 1H), 2.15 (s, 3H). LC-MS (ES, m/z): 574[M+H]⁺.

Example 139: ¹H NMR (400 MHz, Chloroform-d) δ 10.09 (s, 1H), 9.18 (s, 1H), 8.96 (d, J=4.8 Hz, 2H), 8.82 (s, 1H), 8.70 (s, 1H), 7.80 (d, J=2.8 Hz, 1H), 7.44 (t, J=4.4 Hz, 1H), 7.00 (t, J=60.4 Hz, 1H), 6.90 (d, J=2.8 Hz, 1H), 6.31 (d, J=5.2 Hz, 1H), 4.62-4.66 (m, 1H), 3.38-3.42 (m, 1H), 2.88-2.94 (m, 1H), 2.12 (s, 3H). LC-MS (ES, m/z): 574[M+H]⁺.

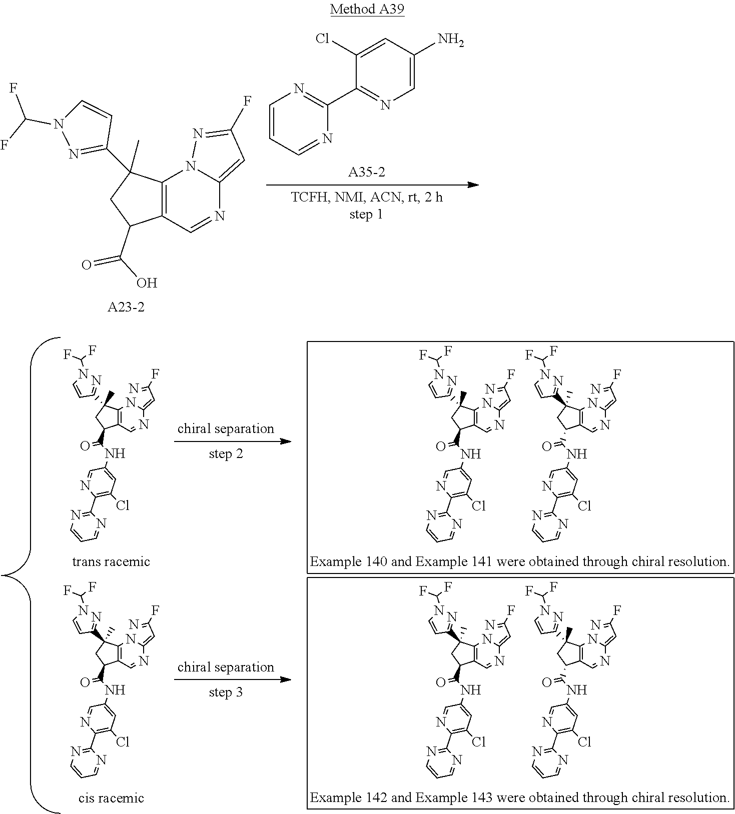

### Examples 140, 141: (6S,8S)—N-(5-chloro-6-(pyrimidin-2-yl)pyridin-3-yl)-8-(1-(difluoromethyl)-1H-pyrazol-3-yl)-2-fluoro-8-methyl-7,8-dihydro-6H-cyclopenta[e]pyrazolo[1,5-a]pyrimidine-6-carboxamide; (6R,8R)—N-(5-chloro-6-(pyrimidin-2-yl)pyridin-3-yl)-8-(1-(difluoromethyl)-1H-pyrazol-3-yl)-2-fluoro-8-methyl-7,8-dihydro-6H-cyclopenta[e]pyrazolo[1,5-a]pyrimidine-6-carboxamide

### Examples 142, 143: (6S,8R)—N-(5-chloro-6-(pyrimidin-2-yl)pyridin-3-yl)-8-(1-(difluoromethyl)-1H-pyrazol-3-yl)-2-fluoro-8-methyl-7,8-dihydro-6H-cyclopenta[e]pyrazolo[1,5-a]pyrimidine-6-carboxamide; (6R,8S)—N-(5-chloro-6-(pyrimidin-2-yl)pyridin-3-yl)-8-(1-(difluoromethyl)-1H-pyrazol-3-yl)-2-fluoro-8-methyl-7,8-dihydro-6H-cyclopenta[e]pyrazolo[1,5-a]pyrimidine-6-carboxamide

The relative and absolute stereochemistry of each example was not determined.

### Step 1: (trans)-N-(5-chloro-6-(pyrimidin-2-yl)pyridin-3-yl)-8-(1-(difluoromethyl)-11H-pyrazol-3-yl)-2-fluoro-8-methyl-7,8-dihydro-6H-cyclopenta[e]pyrazolo[1,5-a]pyrimidine-6-carboxamide and (cis)-N-(5-chloro-6-(pyrimidin-2-yl)pyridin-3-yl)-8-(1-(difluoromethyl)-1H-pyrazol-3-yl)-2-fluoro-8-methyl-7,8-dihydro-6H-cyclopenta[e]pyrazolo[1,5-a]pyrimidine-6-carboxamide

In analogy to Method A1 step 8 but using 8-(1-(difluoromethyl)-1H-pyrazol-3-yl)-2-fluoro-8-methyl-7,8-dihydro-6H-cyclopenta[e]pyrazolo[1,5-a]pyrimidine-6-carboxylic acid (A23-2, 150 mg, 427 mol) and 6-(pyrimidin-2-yl)-5-(trifluoromethyl)pyridin-3-amine (A35-2, 132 mg, 640 mmol), racemic mixtures of the title compounds were obtained: trans isomer (45 mg, 19% yield) as a white solid, LC-MS (ES, m/z): 540[M+H]⁺; cis-isomer (10 mg, 4% yield) as a white solid, LC-MS (ES, m/z): 540[M+H]⁺.

### Step 2: Separation of Enantiomers to Obtain Example 140 and Example 141

45 mg of (trans)-N-(5-chloro-6-(pyrimidin-2-yl)pyridin-3-yl)-8-(1-(difluoromethyl)-1H-pyrazol-3-yl)-2-fluoro-8-methyl-7,8-dihydro-6H-cyclopenta[e]pyrazolo[1,5-a]pyrimidine-6-carboxamide were submitted to chiral HPLC purification (Column: CHIRAL ART Amylose-SA, 2*25 cm, 5 m; Mobile Phase A: Hex (0.1% FA)-HPLC, Mobile Phase B: EtOH-HPLC; Flow rate: 20 ML/MIN mL/min; Gradient: isocratic 60% Mobile Phase B; Wave Length: 254/220 nm nm; RT1 (min): 4.274; RT2 (min): 6.805; Sample Solvent: EtOH-HPLC; Injection Volume: 0.8 mL; Number Of Runs: 5). The second eluting isomer was concentrated and lyophilized to afford Example 140 (8.2 mg, 17% yield) as a white solid. The first eluting isomer was concentrated and lyophilized to afford Example 141 (10.1 mg, 20% yield) as a white solid.

Example 140: ¹H NMR (400 MHz, Chloroform-d) δ 8.97 (d, J=5.2 Hz, 2H), 8.76 (s, 1H), 8.71 (s, 1H), 8.58 (s, 1H), 8.29 (s, 1H), 7.74 (d, J=2.8 Hz, 1H), 7.40 (t, J=4.8 Hz, 1H), 7.06 (t, J=60.8 Hz, 1H), 6.77 (d, J=2.8 Hz, 1H), 6.28 (d, J=5.2 Hz, 1H), 4.55 (t, J=8.4 Hz, 1H), 3.44-3.45 (m, 1H), 2.59-2.64 (m, 1H), 2.16 (s, 3H). LC-MS (ES, m/z): 540[M+H]⁺.

Example 141: ¹H NMR (400 MHz, Chloroform-d) δ 8.97 (d, J=4.8 Hz, 2H), 8.77 (s, 1H), 8.73 (s, 1H), 8.59 (s, 1H), 8.26 (s, 1H), 7.74 (d, J=2.8 Hz, 1H), 7.40 (t, J=4.8 Hz, 1H), 7.06 (t, J=60.8 Hz, 1H), 6.77 (d, J=2.8 Hz, 1H), 6.28 (d, J=4.8 Hz, 1H), 4.56 (t, J=8.4 Hz, 1H), 3.40-3.46 (m, 1H), 2.59-2.65 (m, 1H), 2.16 (s, 3H). LC-MS (ES, m/z): 540[M+H]⁺.

### Step 3: Separation of Enantiomers to Obtain Example 142 and Example 143

20 mg of (cis)-N-(5-chloro-6-(pyrimidin-2-yl)pyridin-3-yl)-8-(1-(difluoromethyl)-1H-pyrazol-3-yl)-2-fluoro-8-methyl-7,8-dihydro-6H-cyclopenta[e]pyrazolo[1,5-a]pyrimidine-6-carboxamide were submitted to chiral HPLC purification (Column: CHIRALPAK IG, 2*25 cm, 5 μm; Mobile Phase A: Hex (0.1% FA)-HPLC, Mobile Phase B: EtOH: DCM=1:1-HPLC; Flow rate: 20 ML/MIN mL/min; Gradient: isocratic 50% Mobile Phase B; Wave Length: 254/220 nm nm; RT1 (min): 8.321; RT2 (min): 10.659; Sample Solvent: EtOH-HPLC; Injection Volume: 1.0 mL; Number Of Runs: 2). The second eluting isomer was concentrated and lyophilized to afford Example 142 (2.8 mg, 27% yield) as a white solid. The first eluting isomer was concentrated and lyophilized to afford Example 143 (2.4 mg, 23% yield) as a white solid.

Example 142: ¹H NMR (400 MHz, Chloroform-d) δ 9.57 (s, 1H), 8.96 (d, J=4.8 Hz, 2H), 8.69 (s, 1H), 8.66 (s, 1H), 8.53 (s, 1H), 7.81 (d, J=2.8 Hz, 1H), 7.39 (t, J=4.8 Hz, 1H), 7.01 (t, J=60.8 Hz, 1H), 6.93 (d, J=2.4 Hz, 1H), 6.30 (d, J=5.2 Hz, 1H), 4.54-4.60 (m, 1H), 3.35-3.40 (m, 1H), 2.86-2.92 (m, 1H), 2.13 (s, 3H). LC-MS (ES, m/z): 540[M+H]⁺.

Example 143: ¹H NMR (400 MHz, Chloroform-d) δ 10.23 (s, 1H), 8.92-8.99 (m, 3H), 8.78 (s, 1H), 8.67 (s, 1H), 7.79 (d, J=2.4 Hz, 1H), 7.42 (t, J=4.8 Hz, 1H), 7.02 (t, J=60.8 Hz, 1H), 6.85 (d, J=2.8 Hz, 1H), 6.29 (d, J=5.2 Hz, 1H), 4.59-4.66 (m, 1H), 3.34-3.38 (m, 1H), 2.88-2.94 (m, 1H), 2.10 (s, 3H). LC-MS (ES, m/z): 540[M+H]⁺.

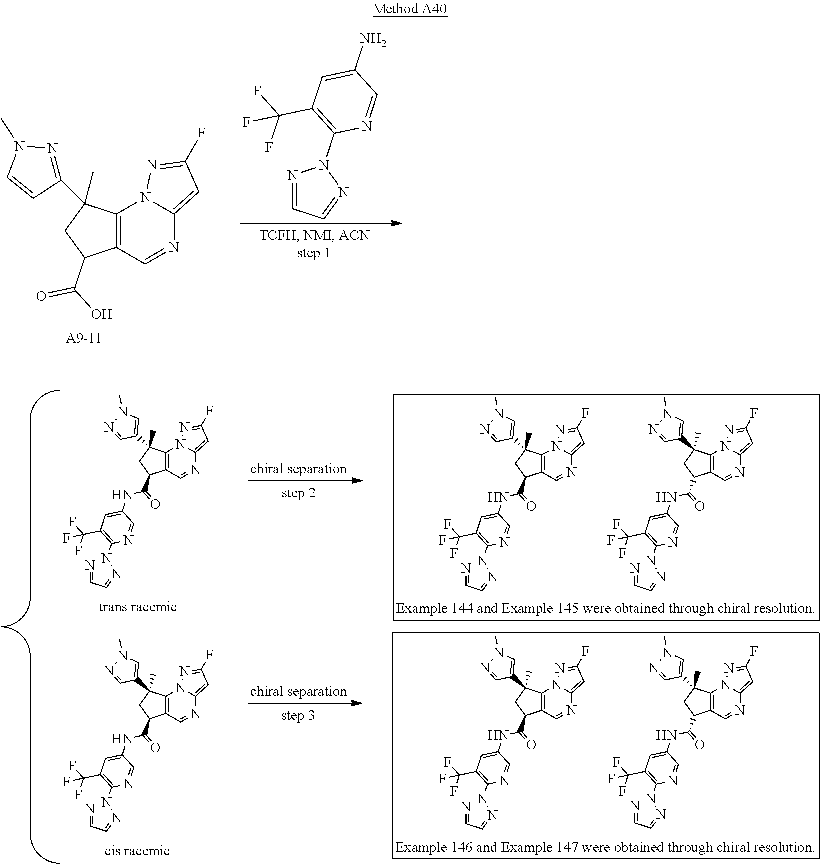

### Examples 144, 145: (6S,8S)—N-(6-(2H-1,2,3-triazol-2-yl)-5-(trifluoromethyl)pyridin-3-yl)-2-fluoro-8-methyl-8-(1-methyl-1H-pyrazol-3-yl)-7,8-dihydro-6H-cyclopenta[e]pyrazolo[1,5-a]pyrimidine-6-carboxamide, (6R,8R)—N-(6-(2H-1,2,3-triazol-2-yl)-5-(trifluoromethyl)pyridin-3-yl)-2-fluoro-8-methyl-8-(1-methyl-1H-pyrazol-3-yl)-7,8-dihydro-6H-cyclopenta[e]pyrazolo[1,5-a]pyrimidine-6-carboxamide

### Examples 146, 147: (6S,8R)—N-(6-(2H-1,2,3-triazol-2-yl)-5-(trifluoromethyl)pyridin-3-yl)-2-fluoro-8-methyl-8-(1-methyl-1H-pyrazol-3-yl)-7,8-dihydro-6H-cyclopenta[e]pyrazolo[1,5-a]pyrimidine-6-carboxamide; (6R,8S)—N-(6-(2H-1,2,3-triazol-2-yl)-5-(trifluoromethyl)pyridin-3-yl)-2-fluoro-8-methyl-8-(1-methyl-1H-pyrazol-3-yl)-7,8-dihydro-6H-cyclopenta[e]pyrazolo[1,5-a]pyrimidine-6-carboxamide

The relative and absolute stereochemistry of each example was not determined.

### Step 1: Trans-N-(6-(2H-1,2,3-triazol-2-yl)-5-(trifluoromethyl)pyridin-3-yl)-2-fluoro-8-methyl-8-(1-methyl-1H-pyrazol-3-yl)-7,8-dihydro-6H-cyclopenta[e]pyrazolo[1,5-a]pyrimidine-6-carboxamide and cis-N-(6-(2H-1,2,3-triazol-2-yl)-5-(trifluoromethyl)pyridin-3-yl)-2-fluoro-8-methyl-8-(1-methyl-1H-pyrazol-3-yl)-7,8-dihydro-6H-cyclopenta[e]pyrazolo[1,5-a]pyrimidine-6-carboxamide

In analogy to Method A1 step 8 but using 2-fluoro-8-methyl-8-(1-methyl-1H-pyrazol-3-yl)-7,8-dihydro-6H-cyclopenta[e]pyrazolo[1,5-a]pyrimidine-6-carboxylic acid (A9-11, 85 mg, 270 mol) and 6-(2H-1,2,3-triazol-2-yl)-5-(trifluoromethyl)pyridin-3-amine (124 mg, 539 mol), racemic mixtures of the title compounds were obtained: trans isomer (20 mg, 14% yield) as a white solid, LC-MS: m/z 527 [M+H]⁺; cis-isomer (6 mg, 4% yield) as a white solid, LC-MS: m/z 527 [M+H]⁺.

### Step 2: Separation of Enantiomers to Obtain Example 144 and Example 145

20 mg of trans-N-(6-(2H-1,2,3-triazol-2-yl)-5-(trifluoromethyl)pyridin-3-yl)-2-fluoro-8-methyl-8-(1-methyl-1H-pyrazol-3-yl)-7,8-dihydro-6H-cyclopenta[e]pyrazolo[1,5-a]pyrimidine-6-carboxamide were submitted to chiral HPLC purification (Column: CHIRALPAK IF, 2*25 cm, m; Mobile Phase A: Hex: DCM=3:1(0.1% FA)-HPLC, Mobile Phase B: EtOH-HPLC; Flow rate: 20 ML/MIN mL/min; Gradient: isocratic 30% Mobile Phase B; Wave Length: 254/220 nm nm; RT1 (min): 5.22; RT2 (min): 7.789; Sample Solvent: EtOH-HPLC; Sample concentration: mg/mL; Injection Volume: 2.0 mL; Number Of Runs: 2). The second eluting isomer was concentrated and lyophilized to afford Example 144 (7.6 mg, 38% yield) as a white solid. The first second eluting isomer was concentrated and lyophilized to afford Example 145 (6.6 mg, 33% yield) as a white solid.

Example 144: ¹H NMR (400 MHz, Chloroform-d) δ 8.90 (d, J=2.4 Hz, 1H), 8.88 (d, J=2.4 Hz, 1H), 8.57 (s, 1H), 8.29 (s, 1H), 7.97 (s, 2H), 7.31 (d, J=2.4 Hz, 1H), 6.60 (d, J=2.4 Hz, 1H), 6.28 (d, J=5.2 Hz, 1H), 4.65 (t, J=8.6 Hz, 1H), 3.87 (s, 3H), 3.47-3.52 (m, 1H), 2.54-2.59 (m, 1H), 2.14 (s, 3H). LC-MS: m/z 527 [M+H]⁺.

Example 145: ¹H NMR (400 MHz, Chloroform-d) δ 8.90 (d, J=2.4 Hz, 1H), 8.88 (d, J=2.4 Hz, 1H), 8.57 (s, 1H), 8.30 (s, 1H), 7.97 (s, 2H), 7.32 (d, J=2.4 Hz, 1H), 6.61 (d, J=2.4 Hz, 1H), 6.28 (d, J=5.2 Hz, 1H), 4.64-4.68 (m, 1H), 3.88 (s, 3H), 3.47-3.52 (m, 1H), 2.54-2.69 (m, 1H), 2.14 (s, 3H). LC-MS: m/z 527 [M+H]⁺.

### Step 3: Separation of Enantiomers to Obtain Example 146 and Example 147

6 mg of cis-N-(6-(2H-1,2,3-triazol-2-yl)-5-(trifluoromethyl)pyridin-3-yl)-2-fluoro-8-methyl-8-(1-methyl-1H-pyrazol-3-yl)-7,8-dihydro-6H-cyclopenta[e]pyrazolo[1,5-a]pyrimidine-6-carboxamide were submitted to chiral HPLC purification (Column: CHIRALPAK IF, 2*25 cm, 5 m; Mobile Phase A: Hex: DCM=3:1(0.1% FA)-HPLC, Mobile Phase B: EtOH-HPLC; Flow rate: 20 ML/MIN mL/min; Gradient: isocratic 10% Mobile Phase B; Wave Length: 254/220 nm nm; RT1 (min): 8.881; RT2 (min): 11.324; Sample Solvent: EtOH-HPLC; Sample concentration: mg/mL; Injection Volume: 2.0 mL; Number Of Runs: 2). The first eluting isomer was concentrated and lyophilized to afford Example 146 (2.4 mg, 39% yield) as a white solid. The second eluting isomer was concentrated and lyophilized to afford Example 147 (2.1 mg, 35% yield) as a white solid.

Example 146: ¹H NMR (400 MHz, Chloroform-d) δ 10.80 (s, 1H), 8.88 (d, J=2.0 Hz, 1H), 8.73 (d, J=2.4 Hz, 1H), 8.63 (s, 1H), 7.94 (s, 2H), 7.40 (d, J=2.0 Hz, 1H), 6.75 (d, J=2.4 Hz, 1H), 6.29 (d, J=5.2 Hz, 1H), 4.55-4.58 (m, 1H), 3.85 (s, 3H), 3.27-3.32 (m, 1H), 2.85-2.91 (m, 1H), 2.13 (s, 3H). LC-MS: m/z 527 [M+H]⁺.

Example 147: ¹H NMR (400 MHz, Chloroform-d) δ 10.80 (s, 111), 8.87 (d, J=2 Hz, 111), 8.73 (d, J=2 Hz, 1H), 8.63 (s, 1H), 7.94 (s, 2H), 7.39 (d, J=2 Hz, 1H), 6.75 (d, J=2.4 Hz, 1H), 6.29 (d, J=4.8 Hz, 1H), 4.55-4.58 (m, 1H), 3.84 (s, 3H), 3.28-3.30 (m, 1H), 2.84-2.91 (m, 1H), 2.13 (s, 3H). LC-MS: m/z 527 [M+H]⁺.

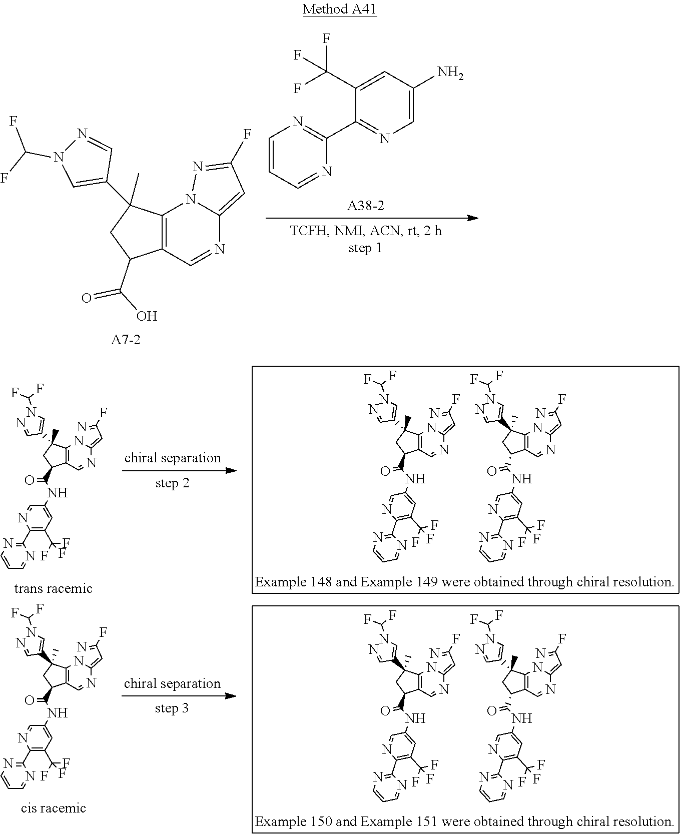

### Examples 148, 149: (6S,8R)-8-(1-(difluoromethyl)-1H-pyrazol-4-yl)-2-fluoro-8-methyl-N-(6-(pyrimidin-2-yl)-5-(trifluoromethyl)pyridin-3-yl)-7,8-dihydro-6H-cyclopenta[e]pyrazolo[1,5-a]pyrimidine-6-carboxamide; (6R,8S)-8-(1-(difluoromethyl)-1H-pyrazol-4-yl)-2-fluoro-8-methyl-N-(6-(pyrimidin-2-yl)-5-(trifluoromethyl)pyridin-3-yl)-7,8-dihydro-6H-cyclopenta[e]pyrazolo[1,5-a]pyrimidine-6-carboxamide

### Examples 150, 151: (6S,8S)-8-(1-(difluoromethyl)-1H-pyrazol-4-yl)-2-fluoro-8-methyl-N-(6-(pyrimidin-2-yl)-5-(trifluoromethyl)pyridin-3-yl)-7,8-dihydro-6H-cyclopenta[e]pyrazolo[1,5-a]pyrimidine-6-carboxamide; (6R,8R)-8-(1-(difluoromethyl)-1H-pyrazol-4-yl)-2-fluoro-8-methyl-N-(6-(pyrimidin-2-yl)-5-(trifluoromethyl)pyridin-3-yl)-7,8-dihydro-6H-cyclopenta[e]pyrazolo[1,5-a]pyrimidine-6-carboxamide

The relative and absolute stereochemistry of each example was not determined.

### Step 1: Trans-8-(1-(difluoromethyl)-1H-pyrazol-4-yl)-2-fluoro-8-methyl-N-(6-(pyrimidin-2-yl)-5-(trifluoromethyl)pyridin-3-yl)-7,8-dihydro-6H-cyclopenta[e]pyrazolo[1,5-a]pyrimidine-6-carboxamide and cis-8-(1-(difluoromethyl)-1H-pyrazol-4-yl)-2-fluoro-8-methyl-N-(6-(pyrimidin-2-yl)-5-(trifluoromethyl)pyridin-3-yl)-7,8-dihydro-6H-cyclopenta[e]pyrazolo[1,5-a]pyrimidine-6-carboxamide

In analogy to Method A1 step 8 but using 8-(1-(difluoromethyl)-1H-pyrazol-4-yl)-2-fluoro-8-methyl-7,8-dihydro-6H-cyclopenta[e]pyrazolo[1,5-a]pyrimidine-6-carboxylic acid (A7-2, 120 mg, 342 mol) and 6-(pyrimidin-2-yl)-5-(trifluoromethyl)pyridin-3-amine (A38-2, 123 mg, 512 mol), racemic mixtures of the title compounds were obtained: trans isomer (20 mg, 10% yield) as an off-white solid, LC-MS: m/z 574 [M+H]⁺; cis-isomer (18 mg, 9% yield) as an off-white solid, LC-MS: m/z 574 [M+H]⁺.

### Step 2: Separation of Enantiomers to Obtain Example 148 and Example 149

17 mg of trans-8-(1-(difluoromethyl)-1H-pyrazol-4-yl)-2-fluoro-8-methyl-N-(6-(pyrimidin-2-yl)-5-(trifluoromethyl)pyridin-3-yl)-7,8-dihydro-6H-cyclopenta[e]pyrazolo[1,5-a]pyrimidine-6-carboxamide were submitted to chiral HPLC purification (Column: CHIRALPAK ID, 2*25 cm, 5 m; Mobile Phase A: Hex: DCM=3:1(0.1% FA)-HPLC, Mobile Phase B: EtOH-HPLC; Flow rate: 20 ML/MIN mL/min; Gradient: isocratic 50% Mobile Phase B; Wave Length: 254/220 nm nm; RT1 (min): 8.34; RT2 (min): 14.14; Sample Solvent: EtOH-HPLC; Injection Volume: 1.55 mL; Number Of Runs: 1). The second eluting isomer was concentrated and lyophilized to afford Example 148 (2.6 mg, 15% yield) as a white solid. The first eluting isomer was concentrated and lyophilized to afford Example 149 (2.0 mg, 11% yield) as a white solid.

Example 148: ¹H NMR (400 MHz, DMSO-d₆) δ 11.12 (s, 1H), 9.07 (d, J=2.4 Hz, 1H), 8.97 (d, J=4.8 Hz, 2H), 8.73 (s, 1H), 8.71 (d, J=2.4 Hz, 1H), 8.22 (s, 1H), 7.84 (s, 1H), 7.73 (t, J=59.2 Hz, 1H), 7.64 (t, J=5.2 Hz, 1H), 6.60 (d, J=4.8 Hz, 1H), 4.58 (t, J=8.2 Hz, 1H), 2.96 (dd, J=13.2, 8.0 Hz, 1H), 2.61 (dd, J=13.2, 8.0 Hz, 1H), 2.00 (s, 3H). LC-MS: m/z 574 [M+H]⁺.

Example 149: ¹H NMR (400 MHz, DMSO-d₆) δ 11.16 (s, 1H), 9.07 (d, J=2.3 Hz, 1H), 8.97 (d, J=5.2 Hz, 2H), 8.73 (s, 1H), 8.71 (d, J=2.4 Hz, 1H), 8.22 (s, 1H), 7.85 (s, 1H), 7.73 (t, J=59.2 Hz, 1H), 7.64 (t, J=5.2 Hz, 1H), 6.60 (d, J=4.8 Hz, 1H), 4.59 (t, J=8.0 Hz, 1H), 2.96 (dd, J=13.2, 8.0 Hz, 1H), 2.61 (dd, J=13.2, 8.0 Hz, 1H), 2.01 (s, 3H). LC-MS: m/z 574 [M+H]⁺.

### Step 3: Separation of Enantiomers to Obtain Example 150 and Example 151

18 mg of cis-8-(1-(difluoromethyl)-1H-pyrazol-4-yl)-2-fluoro-8-methyl-N-(6-(pyrimidin-2-yl)-5-(trifluoromethyl)pyridin-3-yl)-7,8-dihydro-6H-cyclopenta[e]pyrazolo[1,5-a]pyrimidine-6-carboxamide were submitted to chiral HPLC purification (Column: CHIRAL ART Cellulose-SC, 2*25 cm, 5 μm; Mobile Phase A: Hex: DCM=3:1(0.1% FA)-HPLC, Mobile Phase B: EtOH-HPLC; Flow rate: 20 ML/MIN mL/min; Gradient: isocratic 50% Mobile Phase B; Wave Length: 254/220 nm nm; RT1 (min): 5.44; RT2 (min): 10.21; Sample Solvent: EtOH-HPLC; Injection Volume: 0.9 mL; Number Of Runs: 2). The first eluting isomer was concentrated and lyophilized to afford Example 150 (2.2 mg, 12% yield) as a white solid. The second eluting isomer was concentrated and lyophilized to afford Example 151 (2.3 mg, 12% yield) as a white solid.

Example 150: ¹H NMR (400 MHz, DMSO-d₆) δ 11.09 (s, 1H), 9.06 (d, J=2.3 Hz, 1H), 8.97 (d, J=4.8 Hz, 2H), 8.77 (s, 1H), 8.67 (d, J=2.3 Hz, 1H), 8.19 (s, 1H), 7.78 (s, 1H), 7.73 (t, J=59.2 Hz, 1H), 7.64 (t, J=4.8 Hz, 1H), 6.59 (d, J=4.4 Hz, 1H), 4.55-4.60 (m, 1H), 2.84-2.90 (m, 1H), 2.74-2.80 (m, 1H), 1.92 (s, 3H). LC-MS: m/z 574 [M+H]⁺.

Example 151: ¹H NMR (400 MHz, DMSO-d₆) δ 11.09 (s, 1H), 9.06 (d, J=2.3 Hz, 1H), 8.97 (d, J=5.2 Hz, 2H), 8.77 (s, 1H), 8.66 (d, J=2.3 Hz, 1H), 8.19 (s, 1H), 7.79 (s, 1H), 7.73 (t, J=59.2 Hz, 1H), 7.64 (t, J=4.8 Hz, 1H), 6.58 (d, J=4.8 Hz, 1H), 4.56-4.60 (m, 1H), 2.84-2.88 (m, 1H), 2.74-2.80 (m, 1H), 1.92 (s, 3H). LC-MS: mi/z 574 [M+H]⁺.

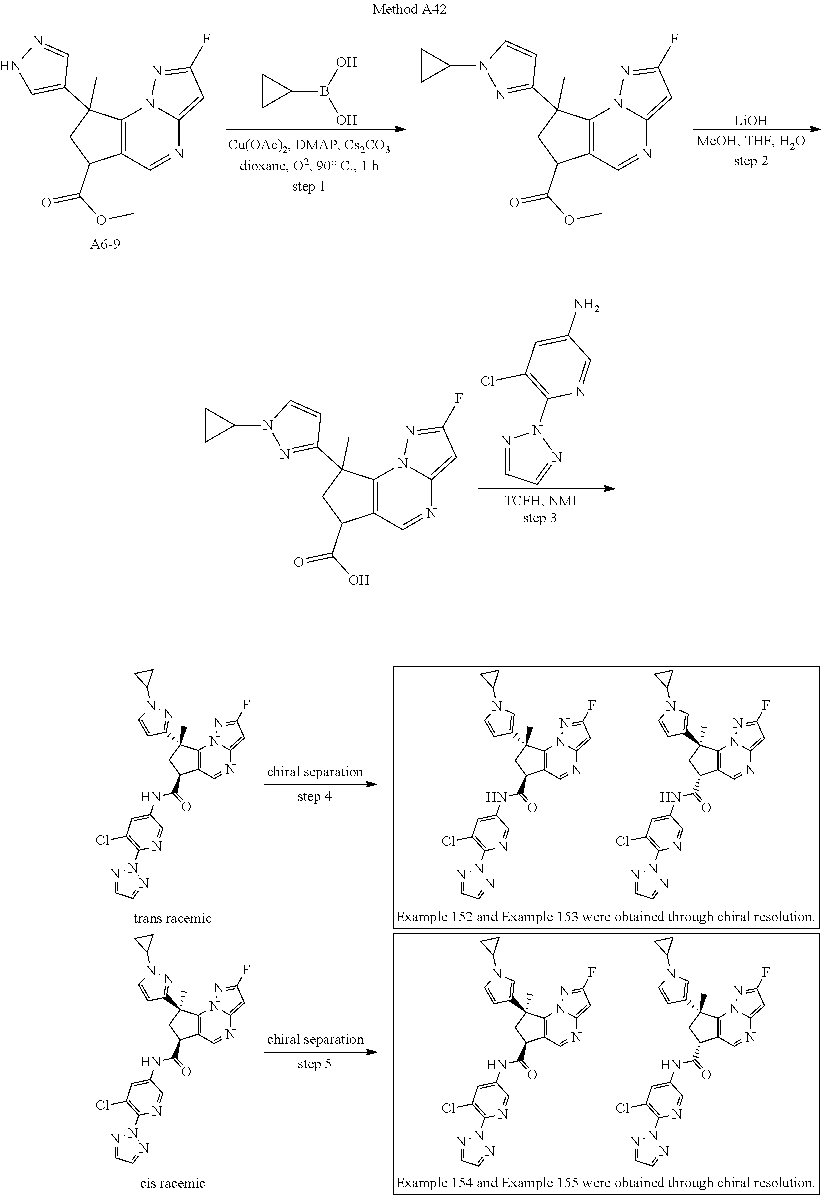

### Examples 152, 153: (6S,8S)—N-(5-chloro-6-(2H-1,2,3-triazol-2-yl)pyridin-3-yl)-8-(1-cyclopropyl-1H-pyrazol-3-y)-2-fluoro-8-methyl-7,8-dihydro-6H-cyclopenta[e]pyrazolo[1,5-a]pyrimidine-6-carboxamide; (6R,8R)—N-(5-chloro-6-(2H-1,2,3-triazol-2-yl)pyridin-3-yl)-8-(1-cyclopropyl-1H-pyrazol-3-yl)-2-fluoro-8-methyl-7,8-dihydro-6H-cyclopenta[e]pyrazolo[1,5-a]pyrimidine-6-carboxamide

### Examples 154, 155: (6S,8R)—N-(5-chloro-6-(2H-1,2,3-triazol-2-yl)pyridin-3-yl)-8-(1-cyclopropyl-1H-pyrazol-3-yl)-2-fluoro-8-methyl-7,8-dihydro-6H-cyclopenta[e]pyrazolo[1,5-a]pyrimidine-6-carboxamide; (6R,8S)—N-(5-chloro-6-(2H-1,2,3-triazol-2-yl)pyridin-3-yl)-8-(1-cyclopropyl-1H-pyrazol-3-yl)-2-fluoro-8-methyl-7,8-dihydro-6H-cyclopenta[e]pyrazolo[1,5-a]pyrimidine-6-carboxamide

The relative and absolute stereochemistry of each example was not determined.

### Step 1: Methyl 8-(1-cyclopropyl-1H-pyrazol-3-yl)-2-fluoro-8-methyl-7,8-dihydro-6H-cyclopenta[e]pyrazolo[1,5-a]pyrimidine-6-carboxylate

In analogy to Method A8 step 1 but using methyl 2-fluoro-8-methyl-8-(1H-pyrazol-3-yl)-7,8-dihydro-6H-cyclopenta[e]pyrazolo[1,5-a]pyrimidine-6-carboxylate (A9-9, 280 mg, 622 μmol), the title compound was obtained as a white solid (90 mg, 41% yield). LC-MS: m/z 356 [M+H]⁺.

### Step 2: 8-(1-cyclopropyl-1H-pyrazol-3-yl)-2-fluoro-8-methyl-7,8-dihydro-6H-cyclopenta[e]pyrazolo[1,5-a]pyrimidine-6-carboxylic acid

In analogy to Method A7 step 2 but using methyl 8-(1-cyclopropyl-1H-pyrazol-3-yl)-2-fluoro-8-methyl-7,8-dihydro-6H-cyclopenta[e]pyrazolo[1,5-a]pyrimidine-6-carboxylate (90 mg, 253 mol), the crude title compound was obtained as a white solid (75 mg, 86% yield). LC-MS: m/z 342 [M+H]⁺.

### Step 3: Trans-N-(5-chloro-6-(2H-1,2,3-triazol-2-yl)pyridin-3-yl)-8-(1-cyclopropyl-1H-pyrazol-3-yl)-2-fluoro-8-methyl-7,8-dihydro-6H-cyclopenta[e]pyrazolo[1,5-a]pyrimidine-6-carboxamide and cis-N-(5-chloro-6-(2H-1,2,3-triazol-2-yl)pyridin-3-yl)-8-(1-cyclopropyl-1H-pyrazol-3-yl)-2-fluoro-8-methyl-7,8-dihydro-6H-cyclopenta[e]pyrazolo[1,5-a]pyrimidine-6-carboxamide

In analogy to Method A1 step 8 but using 8-(1-cyclopropyl-1H-pyrazol-3-yl)-2-fluoro-8-methyl-7,8-dihydro-6H-cyclopenta[e]pyrazolo[1,5-a]pyrimidine-6-carboxylic acid (75 mg, 131.8 mmol) and 5-chloro-6-(2H-1,2,3-triazol-2-yl)pyridin-3-amine (31 mg, 158 mol), racemic mixtures of the title compounds were obtained: trans isomer (40 mg, 58% yield) as a white solid, LC-MS: m/z 519 [M+H]⁺; cis-isomer (20 mg, 29% yield) as a white solid, LC-MS: m/z 519 [M+H]⁺.

### Step 4: Separation of Enantiomers to Obtain Example 152 and Example 153

40 mg of trans-N-(5-chloro-6-(2H-1,2,3-triazol-2-yl)pyridin-3-yl)-8-(1-cyclopropyl-1H-pyrazol-3-yl)-2-fluoro-8-methyl-7,8-dihydro-6H-cyclopenta[e]pyrazolo[1,5-a]pyrimidine-6-carboxamide were submitted to chiral HPLC purification (Column: CHIRALPAK IF, 2*25 cm, 5 μm; Mobile Phase A: Hex: DCM=3:1(0.1% FA)-HPLC, Mobile Phase B: EtOH-HPLC; Flow rate: 18 ML/MIN mL/min; Gradient: isocratic 50% Mobile Phase B; Wave Length: 254/220 nm nm; RT1 (min): 7.56; RT2 (min): 12.048; Sample Solvent: EtOH-HPLC; Injection Volume: 1.5 mL; Number Of Runs: 3). The second eluting isomer was concentrated and lyophilized to afford Example 152 (17.2 mg, 43% yield) as a white solid. The first eluting isomer was concentrated and lyophilized to afford Example 153 (17.3 mg, 43% yield) as a white solid.

Example 152: ¹H NMR (400 MHz, DMSO-d₆) δ:11.13 (s, 1H), 8.75 (d, J=2.0 Hz, 1H), 8.68 (s, 1H), 8.61 (d, J=2.0 Hz, 1H), 8.17 (s, 2H), 7.67 (d, J=2.4 Hz, 1H), 6.55 (d, J=4.8 Hz, 1H), 6.27 (d, J=2.4 Hz, 1H), 4.50-4.54 (m, 1H), 3.52-3.62 (m, 1H), 3.01-3.10 (m, 1H), 2.43-2.51 (m, 1H), 1.98 (s, 3H), 0.82-0.97 (m, 4H). LC-MS: m/z 519 [M+H]⁺.

Example 153: ¹H NMR (400 MHz, DMSO-d₆) δ 11.13 (s, 1H), 8.75 (d, J=2.4 Hz, 1H), 8.68 (s, 1H), 8.61 (d, J=2.4 Hz, 1H), 8.18 (s, 2H), 7.67 (d, J=2.4 Hz, 1H), 6.55 (d, J=4.8 Hz, 1H), 6.27 (d, J=2.4 Hz, 1H), 4.48-4.57 (m, 1H), 3.52-3.62 (m, 1H), 3.00-3.11 (m, 1H), 2.43-2.52 (m, 1H), 1.99 (s, 3H), 0.82-0.97 (m, 4H). LC-MS: m/z 519 [M+H]⁺.

### Step 5: Separation of Enantiomers to Obtain Example 154 and Example 155

20 mg of cis-N-(5-chloro-6-(2H-1,2,3-triazol-2-yl)pyridin-3-yl)-8-(1-cyclopropyl-1H-pyrazol-3-yl)-2-fluoro-8-methyl-7,8-dihydro-6H-cyclopenta[e]pyrazolo[1,5-a]pyrimidine-6-carboxamide were submitted to chiral HPLC purification (Column: CHIRALPAK IF, 2*25 cm, 5 m; Mobile Phase A: Hex: DCM=3:1(0.1% FA)-HPLC, Mobile Phase B: EtOH-HPLC; Flow rate: 20 ML/MIN mL/min; Gradient: isocratic 40% Mobile Phase B; Wave Length: 254/220 nm nm; RT1 (min): 8.556; RT2 (min): 10.156; Sample Solvent: EtOH-HPLC; Injection Volume: 0.7 mL; Number Of Runs: 5). The first eluting isomer was concentrated and lyophilized to afford Example 154 (5.8 mg, 29% yield) as a white solid. The second eluting isomer was concentrated and lyophilized to afford Example 155 (4.5 mg, 22% yield) as a white solid.

Example 154: ¹H NMR (400 MHz, DMSO-d₆) δ 10.96 (s, 1H), 8.73 (d, J=2.0 Hz, 1H), 8.67 (s, 1H), 8.58 (d, J=2.4 Hz, 1H), 8.17 (s, 2H), 7.62 (d, J=2.4 Hz, 1H), 6.54 (d, J=4.8 Hz, 1H), 6.20 (d, J=2.0 Hz, 1H), 4.51-4.55 (m, 1H), 3.47-3.57 (m, 1H), 2.95-3.05 (m, 1H), 2.70-2.80 (m, 1H), 1.88 (s, 3H), 0.72-0.88 (m, 4H). LC-MS: m/z 519 [M+H]⁺.

Example 155: ¹H NMR (400 MHz, DMSO-d₆) δ:10.96 (s, 1H), 8.73 (d, J=2.4 Hz, 1H), 8.67 (s, 1H), 8.58 (d, J=2.0 Hz, 1H), 8.17 (s, 2H), 7.62 (d, J=2.4 Hz, 1H), 6.54 (d, J=5.2 Hz, 1H), 6.20 (d, J=2.4 Hz, 1H), 4.49-4.57 (m, 1H), 3.47-3.58 (m, 1H), 2.95-3.04 (m, 1H), 2.70-2.80 (m, 1H), 1.88 (s, 3H), 0.72-0.89 (m, 4H). LC-MS: m/z 519 [M+H]⁺.

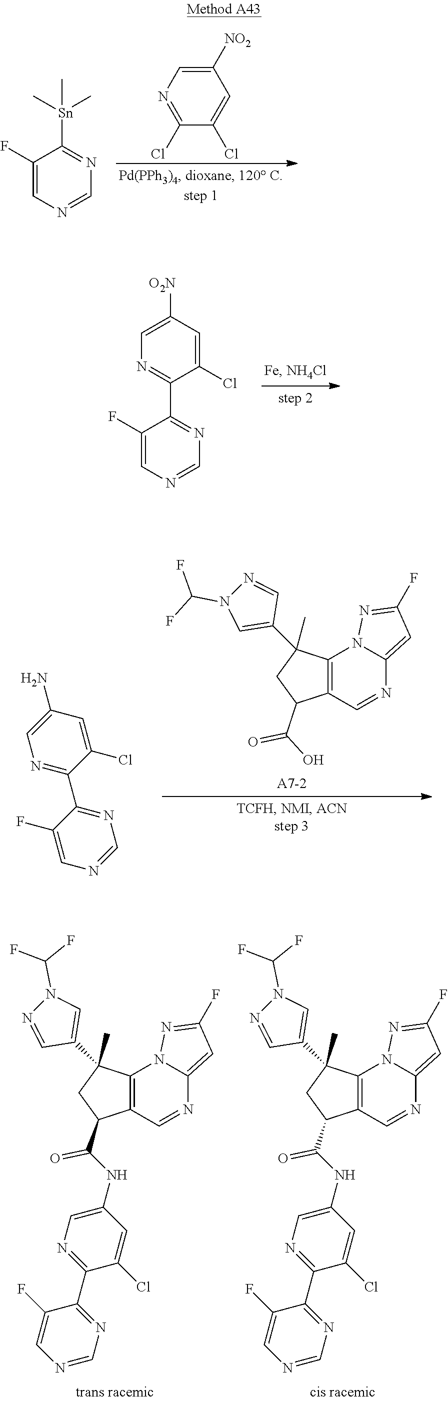

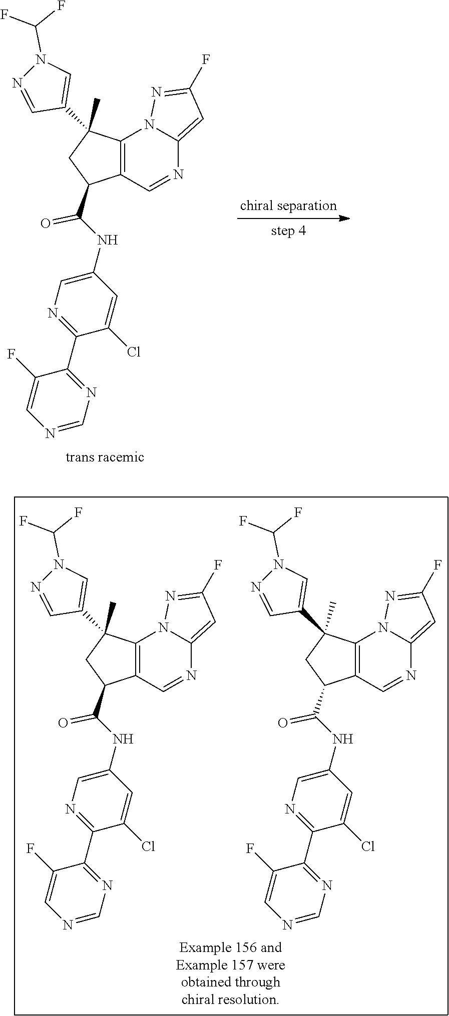

### Examples 156, 157: (6S,8R)—N-(5-chloro-6-(5-fluoropyrimidin-4-yl)pyridin-3-yl)-8-(1-(difluoromethyl)-1H-pyrazol-4-yl)-2-fluoro-8-methyl-7,8-dihydro-6H-cyclopenta[e]pyrazolo[1,5-a]pyrimidine-6-carboxamide; (6R,8S)—N-(5-chloro-6-(5-fluoropyrimidin-4-yl)pyridin-3-yl)-8-(1-(difluoromethyl)-1H-pyrazol-4-yl)-2-fluoro-8-methyl-7,8-dihydro-6H-cyclopenta[e]pyrazolo[1,5-a]pyrimidine-6-carboxamide

The relative and absolute stereochemistry of each example was not determined.

### Step 1: 4-(3-chloro-5-nitropyridin-2-yl)-5-fluoropyrimidine

In analogy to Method A32 step 2 but using 2,3-dichloro-5-nitropyridine (3.4 g, 17.6 mmol) in dioxane (20 mL) and 5-fluoro-4-(trimethylstannyl)pyrimidine (A32-1, 2 g, 7.7 mmol), the title compound was obtained as a yellow solid (1.3 g, 61% yield). LC-MS: m/z 255 [M+H]⁺.

### Step 2: 5-chloro-6-(5-fluoropyrimidin-4-yl)pyridin-3-amine

In analogy to Method A30 step 13 but using 4-(3-chloro-5-nitropyridin-2-yl)-5-fluoropyrimidine (0.2 g, 785 mol), the title compound was obtained as a yellow solid (150 mg, 79% yield). LC-MS: m/z 225 [M+H]⁺.

### Step 3: Trans-N-(5-chloro-6-(5-fluoropyrimidin-4-yl)pyridin-3-yl)-8-(1-(difluoromethyl)-1H-pyrazol-4-yl)-2-fluoro-8-methyl-7,8-dihydro-6H-cyclopenta[c]pyrazolo[1,5-a]pyrimidine-6-carboxamide and cis-N-(5-chloro-6-(5-fluoropyrimidin-4-yl)pyridin-3-yl)-8-(1-(difluoromethyl)-1H-pyrazol-4-yl)-2-fluoro-8-methyl-7,8-dihydro-6H-cyclopenta[e]pyrazolo[1,5-a]pyrimidine-6-carboxamide

In analogy to Method A1 step 8 but using 8-(1-(difluoromethyl)-1H-pyrazol-4-yl)-2-fluoro-8-methyl-7,8-dihydro-6H-cyclopenta[e]pyrazolo[1,5-a]pyrimidine-6-carboxylic acid (A7-2, 90 mg, 256 μmol) and 5-chloro-6-(5-fluoropyrimidin-4-yl)pyridin-3-amine (69 mg, 307 μmol), racemic mixtures of the title compounds were obtained: trans isomer (8.5 mg, 5% yield) as a white solid, LC-MS: m/z 558 [M+H]⁺; cis-isomer (2.8 mg, 2% yield) as a white solid, LC-MS: m/z 558 [M+H]⁺.

### Step 4: Separation of Enantiomers to Obtain Example 156 and Example 157

7 mg of trans-N-(5-chloro-6-(5-fluoropyrimidin-4-yl)pyridin-3-yl)-8-(1-(difluoromethyl)-1H-pyrazol-4-yl)-2-fluoro-8-methyl-7,8-dihydro-6H-cyclopenta[e]pyrazolo[1,5-a]pyrimidine-6-carboxamide were submitted to chiral HPLC purification (Column: CHIRALPAK IG, 2*25 cm, 5 μm; Mobile Phase A: Hex (0.1% FA)-HPLC, Mobile Phase B: EtOH: DCM=1:1-HPLC; Flow rate: 20 ML/MIN mL/min; Gradient: isocratic 50% Mobile Phase B; Wave Length: 254/220 nm nm; RT1 (min): 8.17; RT2 (min): 9.948; Sample Solvent: EtOH-HPLC; Injection Volume: 1.4 mL; Number Of Runs: 1). The second eluting isomer was concentrated and lyophilized to afford Example 156 (2.5 mg, 33% yield) as a white solid. The first eluting isomer was concentrated and lyophilized to afford Example 157 (2.2 mg, 31% yield) as a white solid.

Example 156: ¹H NMR (400 MHz, DMSO-d₆) δ 11.09 (s, 1H), 9.24 (d, J=2.8 Hz, 1H), 9.13 (d, J=1.8 Hz, 1H), 8.86 (d, J=2.1 Hz, 1H), 8.72 (s, 1H), 8.53 (d, J=2.1 Hz, 1H), 8.21 (d, J=2.7 Hz, 1H), 7.70 (t, J=59.1 Hz, 1H), 6.63 (d, J=2.8 Hz, 1H), 6.59 (d, J=4.9 Hz, 1H), 4.52 (t, J=8.1 Hz, 1H), 3.00-3.06 (m, 1H), 2.55-2.60 (m, 1H), 2.04 (s, 3H). LC-MS: m/z 558 [M+H]⁺.

Example 157: ¹H NMR (400 MHz, DMSO-d₆) δ 11.09 (s, 1H), 9.24 (d, J=2.9 Hz, 1H), 9.13 (d, J=1.8 Hz, 1H), 8.86 (d, J=2.1 Hz, 1H), 8.72 (s, 1H), 8.53 (d, J=2.1 Hz, 1H), 8.21 (d, J=2.7 Hz, 1H), 7.70 (t, J=59.1 Hz, 1H), 6.63 (d, J=2.8 Hz, 1H), 6.59 (d, J=4.9 Hz, 1H), 4.52 (t, J=8.1 Hz, 1H), 3.00-3.06 (m, 1H), 2.55-2.61 (m, 1H), 2.04 (s, 3H). LC-MS: m/z 558 [M+H]⁺.

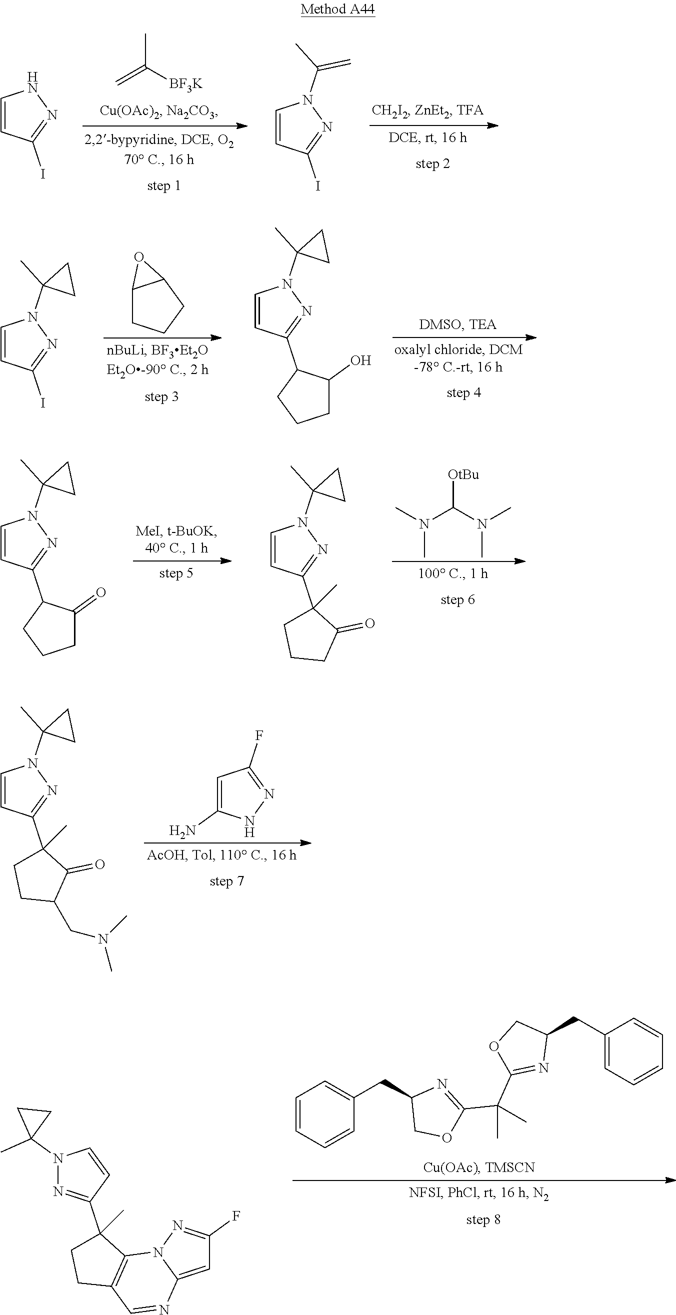

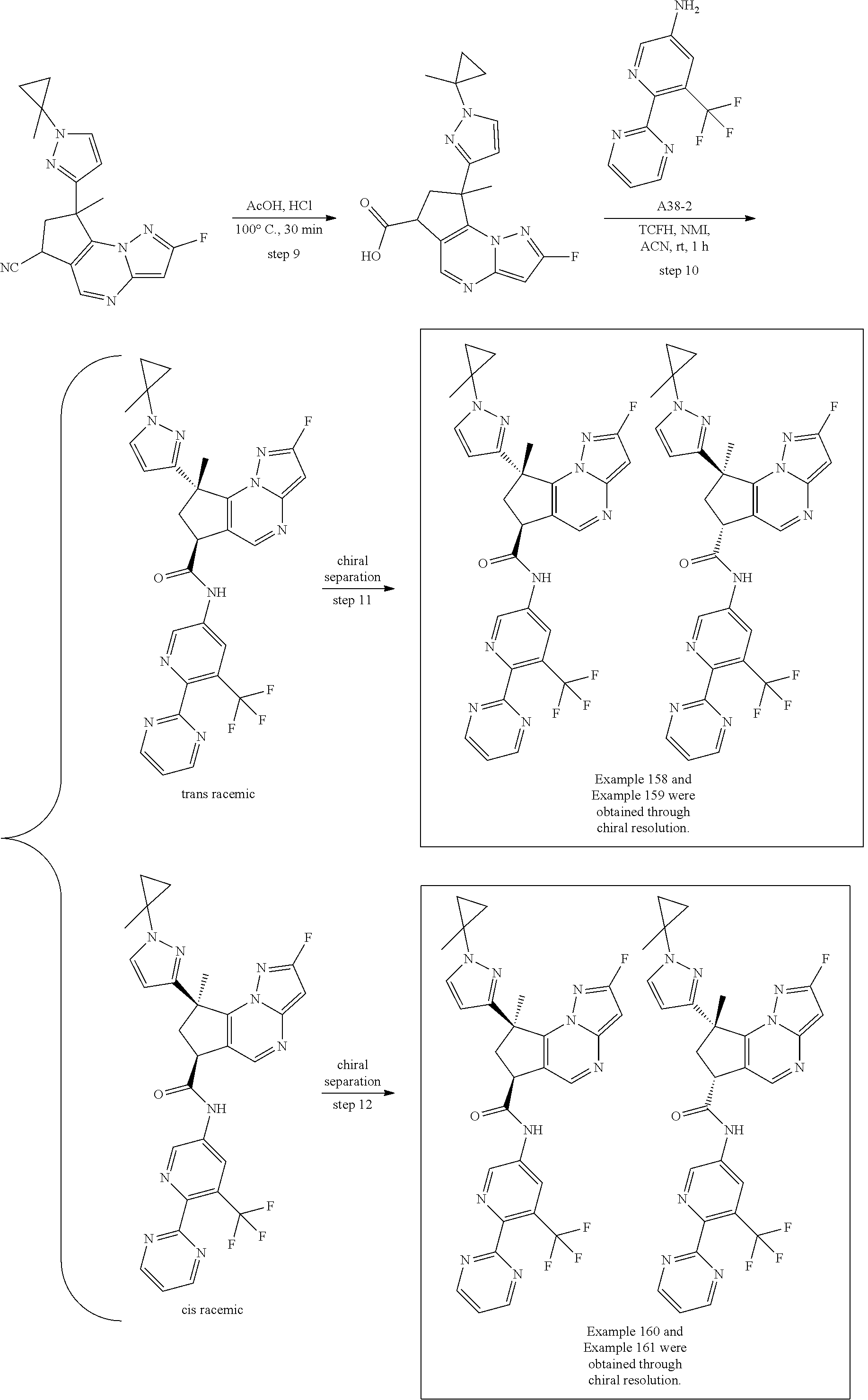

### Examples 158, 159: (6S,8S)-2-fluoro-8-methyl-8-(1-(1-methylcyclopropyl)-1H-pyrazol-3-yl)-N-(6-(pyrimidin-2-yl)-5-(trifluoromethyl)pyridin-3-yl)-7,8-dihydro-6H-cyclopenta[e]pyrazolo[1,5-a]pyrimidine-6-carboxamide; (6R,8R)-2-fluoro-8-methyl-8-(1-(1-methylcyclopropyl)-1H-pyrazol-3-yl)-N-(6-(pyrimidin-2-yl)-5-(trifluoromethyl)pyridin-3-yl)-7,8-dihydro-6H-cyclopenta[e]pyrazolo[1,5-a]pyrimidine-6-carboxamide

### Examples 160, 161: (6S,8R)-2-fluoro-8-methyl-8-(1-(1-methylcyclopropyl)-1H-pyrazol-3-yl)-N-(6-(pyrimidin-2-yl)-5-(trifluoromethyl)pyridin-3-yl)-7,8-dihydro-6H-cyclopenta[e]pyrazolo[1,5-a]pyrimidine-6-carboxamide; (6R,8S)-2-fluoro-8-methyl-8-(1-(1-methylcyclopropyl)-1H-pyrazol-3-yl)-N-(6-(pyrimidin-2-yl)-5-(trifluoromethyl)pyridin-3-yl)-7,8-dihydro-6H-cyclopenta[e]pyrazolo[1,5-a]pyrimidine-6-carboxamide

The relative and absolute stereochemistry of each example was not determined.

### Step 1: 3-iodo-1-(prop-1-en-2-yl)-1H-pyrazole

In analogy to Method A28 step 1 but using 3-iodo-1H-pyrazole (20 g, 103.1 mmol) and stirring at 70° C. for 16 h, the title compound was obtained as a yellow oil (7.3 g, 30% yield). ¹H NMR (400 MHz, DMSO-d₆) δ: 8.02 (d, J=2.8 Hz, 1H), 6.64 (d, J=2.4 Hz, 1H), 5.37 (s, 1H), 4.71-4.76 (m, 1H), 2.18-2.23 (m, 3H). LC-MS (ES, m/z): 235[M+H]⁺.

### Step 2: 3-iodo-1-(1-methylcyclopropyl)-11H-pyrazole

In analogy to Method A28 step 2 but using 3-iodo-1-(prop-1-en-2-yl)-1H-pyrazole (7.3 g, 31.2 mmol), the title compound was obtained as a yellow solid (4.3 g, 55% yield). ¹H NMR (400 MHz, DMSO-d₆) δ: 7.71 (d, J=1.6 Hz, 1H), 6.42 (d, J=1.6 Hz, 1H), 1.53 (s, 3H), 1.12-1.17 (m, 2H), 0.86-0.91 (m, 2H). LC-MS (ES, m/z): 249[M+H]⁺.

### Step 3: 2-(1-(1-methylcyclopropyl)-1H-pyrazol-3-yl)cyclopentan-1-ol

In analogy to Method A6 step 2 but using 3-iodo-1-(1-methylcyclopropyl)-1H-pyrazole (4.3 g, 17.3 mmol), the title compound was obtained as a yellow oil (0.6 g, 18% yield). LC-MS: m/z 207 [M+H]⁺.

### Step 4: 2-(1-(1-methylcyclopropyl)-1H-pyrazol-3-yl)cyclopentan-1-one

To a stirred solution of oxalyl chloride (2.1 g, 17.0 mmol) in DCM (15 mL) was added DMSO (2.6 g, 33.9 mmol) in DCM (15 mL) under nitrogen at −78° C. The resulting mixture was stirred at −78° C. for 0.5 h. A solution of 2-(1-(1-methylcyclopropyl)-1H-pyrazol-3-yl)cyclopentan-1-ol (1.8 g, 8.5 mmol) in DCM (15 mL) was added slowly at −78° C. under nitrogen, and it was stirred at −78° C. for 30 min. Triethylamine (4.3 g, 42.4 mmol) was added dropwise at −78° C. under nitrogen. The mixture was allowed to warm to 25° C. and was stirred for 16 h. The reaction was quenched with water (30 mL), and the resulting mixture was extracted with DCM (3×30 mL). The combined organic layers were dried over anhydrous sodium sulfate and concentrated under reduced pressure to afford the crude title compound (1.8 g) as a yellow oil, which was used in the next step without further purification. LC-MS: m/z 205 [M+H]⁺.

### Step 5: 2-methyl-2-(1-(1-methylcyclopropyl)-1H-pyrazol-3-yl)cyclopentan-1-one

In analogy to Method A1 step 3 but using 2-(1-(1-methylcyclopropyl)-1H-pyrazol-3-yl)cyclopentan-1-one (370 mg, 1.8 mmol) and purification of the product by reverse phase column chromatography, the title compound was obtained as a yellow oil (220 mg, 55% yield). LC-MS: m/z 219 [M+H]⁺.

### Step 6: 5-((dimethylamino)methylene)-2-methyl-2-(1-(1-methylcyclopropyl)-1H-pyrazol-3-yl)cyclopentan-1-one

In analogy to Method A1 step 4 but using 2-methyl-2-(1-(1-methylcyclopropyl)-1H-pyrazol-3-yl)cyclopentan-1-one (1.2 g, 5.5 mmol) in 1-tert-butoxy-N,N,N′,N′-tetramethyl-methanediamine (1.9 g, 11.0 mmol) and stirring at 100° C. for 1, the crude title compound was obtained as a yellow oil (1.2 g, 79% yield), which was used in the next step without further purification. LC-MS: m/z 274 [M+H]⁺.

### Step 7: 2-fluoro-8-methyl-8-(1-(1-methylcyclopropyl)-1H-pyrazol-3-yl)-7,8-dihydro-6H-cyclopenta[e]pyrazolo[1,5-a]pyrimidine

In analogy to Method A1 step 5 but using 5-((dimethylamino)methylene)-2-methyl-2-(1-(1-methylcyclopropyl)-1H-pyrazol-3-yl)cyclopentan-1-one (1.2 g, 4.4 mmol), the title compound was obtained as a yellow solid (1 g, 73% yield). ¹H NMR (400 MHz, DMSO-d₆) δ: 8.59 (s, 1H), 7.64 (d, J=2.4 Hz, 1H), 6.45 (d, J=4.8 Hz, 1H), 6.14 (d, J=2.4 Hz, 1H), 3.01-3.10 (m, 2H), 2.58-2.69 (m, 1H), 2.20-2.32 (m, 1H), 1.86 (s, 3H), 1.44 (s, 3H), 1.01-1.06 (m, 2H), 0.75-0.83 (m, 2H). LC-MS: m/z 312 [M+H]⁺.

### Step 8: 2-fluoro-8-methyl-8-(1-(1-methylcyclopropyl)-1H-pyrazol-3-yl)-7,8-dihydro-6H-cyclopenta[e]pyrazolo[1,5-a]pyrimidine-6-carbonitrile

In analogy to Method A1 step 6 but using 2-fluoro-8-methyl-8-(1-(1-methylcyclopropyl)-1H-pyrazol-3-yl)-7,8-dihydro-6H-cyclopenta[e]pyrazolo[1,5-a]pyrimidine (1.2 g, 3.8 mmol) in chlorobenzene (125 mL), the title compound was obtained as a yellow solid (340 mg, 13% yield). LC-MS: m/z 337 [M+H]⁺.

### Step 9: 2-fluoro-8-methyl-8-(1-(1-methylcyclopropyl)-1H-pyrazol-3-yl)-7,8-dihydro-6H-cyclopenta[e]pyrazolo[1,5-a]pyrimidine-6-carboxylic acid

In analogy to Method A9 step 8 but using 2-fluoro-8-methyl-8-(1-(1-methylcyclopropyl)-1H-pyrazol-3-yl)-7,8-dihydro-6H-cyclopenta[e]pyrazolo[1,5-a]pyrimidine-6-carbonitrile (340 mg, 505 μmol), the title compound was obtained as a yellow oil (160 mg, 82% yield). LC-MS: m/z 356 [M+H]⁺.

### Step 10: (trans)-2-fluoro-8-methyl-8-(1-(1-methylcyclopropyl)-1H-pyrazol-3-yl)-N-(6-(pyrimidin-2-yl)-5-(trifluoromethyl)pyridin-3-yl)-7,8-dihydro-6H-cyclopenta[e]pyrazolo[1,5-a]pyrimidine-6-carboxamide and (cis)-2-fluoro-8-methyl-8-(1-(1-methylcyclopropyl)-1H-pyrazol-3-yl)-N-(6-(pyrimidin-2-yl)-5-(trifluoromethyl)pyridin-3-yl)-7,8-dihydro-6H-cyclopenta[e]pyrazolo[1,5-a]pyrimidine-6-carboxamide

In analogy to Method A1 step 8 but using 2-fluoro-8-methyl-8-(1-(1-methylcyclopropyl)-1H-pyrazol-3-yl)-7,8-dihydro-6H-cyclopenta[e]pyrazolo[1,5-a]pyrimidine-6-carboxylic acid (150 mg, 422 μmol) and 6-(pyrimidin-2-yl)-5-(trifluoromethyl)pyridin-3-amine (A38-2, 112 mg, 464 μmol), racemic mixtures of the title compounds were obtained: trans isomer (75 mg, 30% yield) as an off-white solid, LC-MS: m/z 578 [M+H]⁺; cis-isomer (30 mg, 12% yield) as an off-white solid, LC-MS: m/z 578 [M+H]⁺.

### Step 11: Separation of Enantiomers to Obtain Example 158 and Example 159

27 mg of (trans)-2-fluoro-8-methyl-8-(1-(1-methylcyclopropyl)-1H-pyrazol-3-yl)-N-(6-(pyrimidin-2-yl)-5-(trifluoromethyl)pyridin-3-yl)-7,8-dihydro-6H-cyclopenta[e]pyrazolo[1,5-a]pyrimidine-6-carboxamide were submitted to chiral HPLC purification (Column: CHIRALPAK IF, 2*25 cm, 5 m; Mobile Phase A: Hex (0.1% FA)-HPLC, Mobile Phase B: EtOH: DCM=1:1-HPLC; Flow rate: 20 mL/min; Gradient: isocratic 60% Mobile Phase B; Wave Length: 254/220 nm; RT1 (min): 4.753; RT2 (min): 7.829; Sample Solvent: EtOH-HPLC; Injection Volume: 1.2 mL; Number Of Runs: 3). The second eluting isomer was concentrated and lyophilized to afford Example 158 (22.5 mg, 29% yield) as a white solid. The first eluting isomer was concentrated and lyophilized to afford Example 159 (30.2 mg, 40% yield) as a white solid.

Example 158: ¹H NMR (400 MHz, DMSO-d₆) δ: 11.11 (s, 1H), 9.09 (d, J=2.0 Hz, 1H), 8.98 (d, J=4.8 Hz, 2H), 8.73 (d, J=2.0 Hz, 1H), 8.71 (s, 1H), 7.71 (d, J=2.0 Hz, 1H), 7.64 (t, J=4.8 Hz, 111), 6.56 (d, J=4.8 Hz, 1H), 6.26 (d, J=2.0 Hz, 1H), 4.53 (t, J=8.4 Hz, 1H), 3.11 (dd, J=12.4, 8.0 Hz, 1H), 2.52-2.55 (m, 1H), 1.99 (s, 3H), 1.47 (s. 3H), 1.01-1.12 (m, 2H), 0.78-0.88 (m, 2H). LC-MS (ES, m/z): 578 [M+H]⁺.

Example 159: ¹H NMR (400 MHz, DMSO-d₆) δ: 11.11 (s, 1H), 9.10 (d, J=2.0 Hz, 1H), 8.98 (d, J=5.2 Hz, 2H), 8.73 (d, J=2.4 Hz, 1H), 8.71 (s, 1H), 7.71 (d, J=2.4 Hz, 1H), 7.64 (t, J=4.8 Hz, 11H), 6.56 (d, J=5.2 Hz, 1H), 6.26 (d, J=2.4 Hz, 1H), 4.53 (t, J=8.0 Hz, 1H), 3.11 (dd, J=12.4, 8.0 Hz, 1H), 2.52-2.55 (m, 1H), 1.99 (s, 3H), 1.47 (s, 3H), 1.01-1.12 (m, 2H), 0.78-0.88 (m, 2H). LC-MS (ES, m/z): 578 [M+H]⁺.

### Step 12: Separation of Enantiomers to Obtain Example 160 and Example 161

30 mg of (cis)-2-fluoro-8-methyl-8-(1-(1-methylcyclopropyl)-1H-pyrazol-3-yl)-N-(6-(pyrimidin-2-yl)-5-(trifluoromethyl)pyridin-3-yl)-7,8-dihydro-6H-cyclopenta[e]pyrazolo[1,5-a]pyrimidine-6-carboxamide were submitted to chiral HPLC purification (Column: CHIRAL ART Cellulose-SZ, 3*25 cm, 5 μm; Mobile Phase A: Hex (0.1% FA)-HPLC, Mobile Phase B: MeOH: DCM=1:1-HPLC; Flow rate: 20 mL/min; Gradient: isocratic 40% Mobile Phase B; Wave Length: 254/220 nm; RT1 (min): 9.733; RT2 (min): 14.084; Sample Solvent: EtOH-HPLC; Injection Volume: 2.0 mL; Number Of Runs: 2). The first eluting isomer was concentrated and lyophilized to afford Example 160 (11.7 mg, 38% yield) as a white solid. The second eluting isomer was concentrated and lyophilized to afford Example 161 (6.8 mg, 22% yield) as a white solid.

Example 160: ¹H NMR (400 MHz, DMSO-d₆) δ:10.93 (s, 1H), 9.07 (d, J=1.6 Hz, 1H), 8.97 (d, J=5.2 Hz, 2H), 8.72 (d, J=2.4 Hz, 1H), 8.69 (s, 1H), 7.62-7.65 (m, 2H), 6.54 (d, J=4.8 Hz, 1H), 6.24 (d, J=2.0 Hz, 1H), 4.54 (dd, J=9.2, 4.8 Hz, 1H), 3.13 (dd, J=13.2, 5.2 Hz, 1H), 2.76 (dd, J=13.2, 9.6 Hz, 1H), 1.89 (s, 3H), 1.35 (s, 3H), 0.88-1.01 (m, 2H), 0.58-0.70 (m, 2H). LC-MS (ES, m/z): 578 [M+H]⁺.

Example 161: ¹H NMR (400 MHz, DMSO-d₆) δ: 10.94 (s, 1H), 9.07 (s, 1H), 8.98 (d, J=4.8 Hz, 2H), 8.72 (s, 1H), 8.69 (s, 1H), 7.60-7.67 (m, 2H), 6.54 (d, J=4.8 Hz, 1H), 6.24 (d, J=2.0 Hz, 1H), 4.54 (dd, J=9.2, 5.2 Hz, 1H), 3.13 (dd, J=12.8, 4.8 Hz, 1H), 2.76 (dd, J=13.2, 9.6 Hz, 1H), 1.89 (s, 3H), 1.35 (s, 3H), 0.88-1.01 (m, 2H), 0.58-0.70 (m, 2H). LC-MS (ES, m/z): 578 [M+H]⁺.

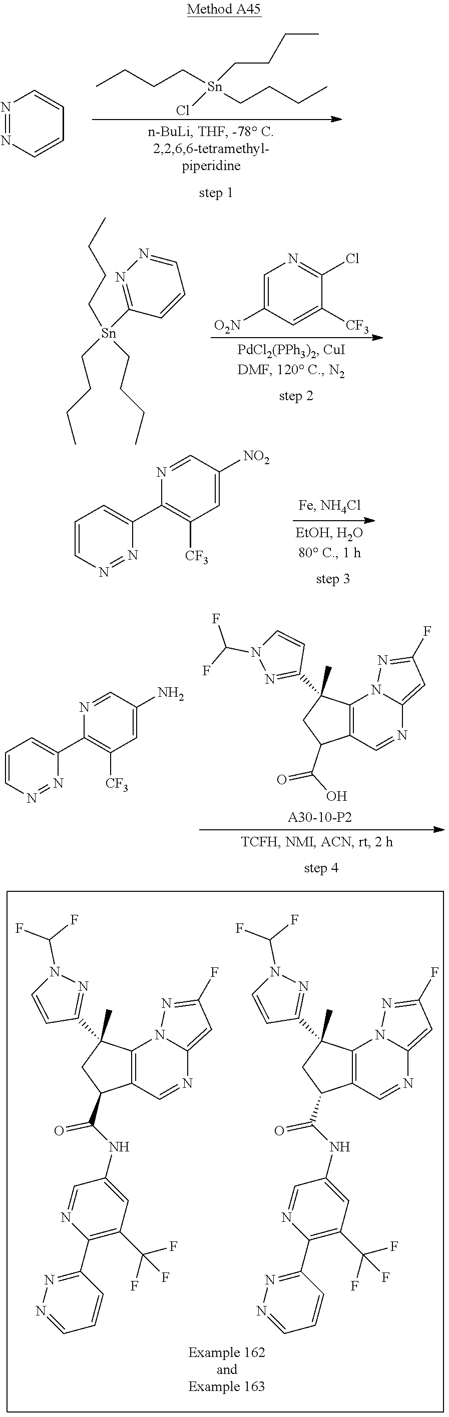

### Examples 162, 163: (6S,8S)-8-(1-(difluoromethyl)-1H-pyrazol-3-yl)-2-fluoro-8-methyl-N-(6-(pyridazin-3-yl)-5-(trifluoromethyl)pyridin-3-yl)-7,8-dihydro-6H-cyclopenta[e]pyrazolo[1,5-a]pyrimidine-6-carboxamide; (6R,8S)-8-(1-(difluoromethyl)-1H-pyrazol-3-yl)-2-fluoro-8-methyl-N-(6-(pyridazin-3-yl)-5-(trifluoromethyl)pyridin-3-yl)-7,8-dihydro-6H-cyclopenta[e]pyrazolo[1,5-a]pyrimidine-6-carboxamide

The relative and absolute stereochemistry of each example was not determined.

### Step 1: 3-(tributylstannyl)pyridazine

To a solution of 2,2,6,6-tetramethylpiperidine (1.94 g, 13.7 mmol, 2.32 mL) in THF (80 mL) was added n-Butyllithium (5.5 mL, 2.5 M in hexanes) at −30° C. The mixture was stirred at 0° C. for 30 min. A solution of pyridazine (1 g, 12.5 mmol) in THF (20 mL) and a solution of tributylchlorostannane (4.1 g, 12.5 mmol) in THF (20 mL) were added simultaneously at −78° C., and the mixture was stirred at −78° C. for 4 h. The mixture was allowed to warm to 25° C. and quenched with aqueous saturated ammonium chloride (20 ml). The mixture was diluted with water (200 mL) and extracted with DCM (3×200 mL). The combined organic layers were dried over anhydrous sodium sulfate and concentrated under reduced pressure. The residue was applied on a silica gel column and eluted with EA/PE (1:1) to afford the title compound (2.1 g, 45% yield) as a yellow oil. LC-MS (ES, m/z): 371 [M+H]⁺.

### Step 2: 3-(5-nitro-3-(trifluoromethyl)pyridin-2-yl)pyridazine

In analogy to Method A30 step 12 but using 3-(tributylstannyl)pyridazine (1 g, 2.7 mmol) and 2-chloro-5-nitro-3-(trifluoromethyl)pyridine (614 mg, 2.7 mmol) and stirring at 120° C. for 1 h, the title compound was obtained as a yellow oil (120 mg, 16% yield). LC-MS (ES, m/z): 271 [M+H]⁺.

### Step 3: 6-(pyridazin-3-yl)-5-(trifluoromethyl)pyridin-3-amine

In analogy to Method A30 step 13 but using 3-(5-nitro-3-(trifluoromethyl)pyridin-2-yl)pyridazine (110 mg, 407 mol), the title compound was obtained as a yellow oil (45 mg, 46% yield). LC-MS (ES, m/z): 241 [M+H]⁺.

### Step 4: Example 162 and Example 163

In analogy to Method A1 step 8 but using (8S)-8-(1-(difluoromethyl)-1H-pyrazol-3-yl)-2-fluoro-8-methyl-7,8-dihydro-6H-cyclopenta[e]pyrazolo[1,5-a]pyrimidine-6-carboxylic acid (A30-10-P2, 44 mg, 125 mol) and 6-(pyridazin-3-yl)-5-(trifluoromethyl)pyridin-3-amine (30 mg, 125 mol), the diastereomeric title compounds were obtained: trans isomer Example 162 (14.0 mg, 19% yield) as a white solid and cis-isomer Example 163 (6.0 mg, 8% yield) as a white solid.

Example 162: ¹H NMR (400 MHz, DMSO-d₆) δ 11.17 (s, 1H), 9.31-9.37 (m, 1H), 9.14 (d, J=2.0 Hz, 1H), 8.77 (d, J=2.4 Hz, 1H), 8.75 (s, 1H), 8.21 (d, J=2.8 Hz, 1H), 8.02-8.06 (m, 1H), 7.86-7.92 (m, 1H), 7.71 (t, J=59.2 Hz, 1H), 6.64 (d, J=2.8 Hz, 1H), 6.59 (d, J=4.8 Hz, 1H), 4.50-4.58 (m, J=8.0 Hz, 1H), 3.00-3.09 (m, 1H), 2.55-2.65 (m, 1H), 2.04 (s, 3H). LC-MS (ES, m/z): 574[M+H]⁺.

Example 163: ¹H NMR (400 MHz, DMSO-d₆) δ 11.07 (s, 1H), 9.31-9.37 (m, 1H), 9.11 (d, J=2.0 Hz, 1H), 8.75 (s, 1H), 8.72 (d, J=2.4 Hz, 2H), 8.14 (d, J=2.8 Hz, 1H), 8.00-8.06 (m, 1H), 7.86-7.93 (m, 1H), 7.70 (t, J=59.2 Hz, 1H), 6.57 (d, J=4.8 Hz, 1H), 6.49 (d, J=2.8 Hz, 1H), 4.54-4.63 (m, 1H), 2.79-2.96 (m, 2H), 1.93 (s, 3H). LC-MS (ES, m/z): 574[M+H]⁺.

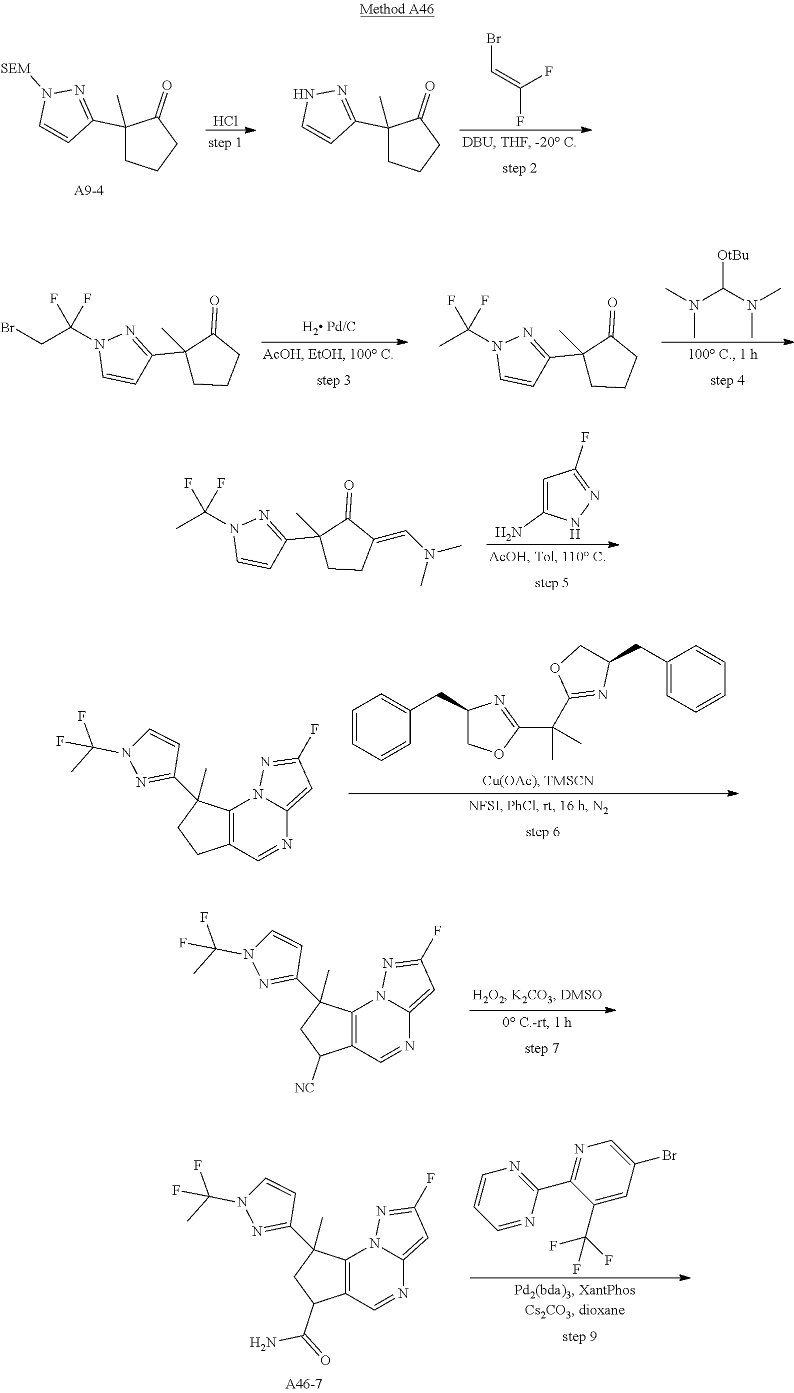

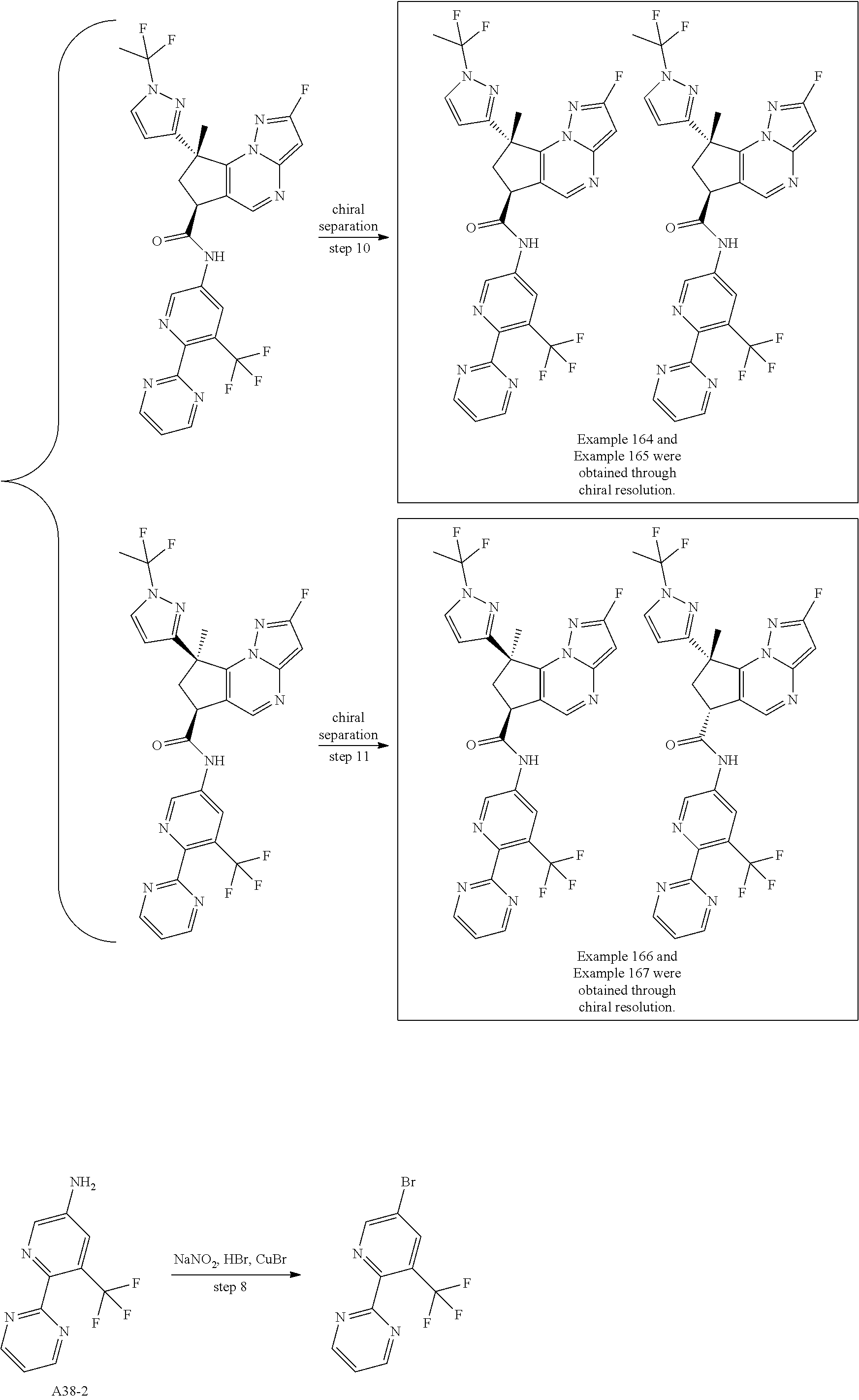

### Examples 164, 165: (6S,8S)-8-(1-(1,1-difluoroethyl)-1H-pyrazol-3-yl)-2-fluoro-8-methyl-N-(6-(pyrimidin-2-yl)-5-(trifluoromethyl)pyridin-3-yl)-7,8-dihydro-6H-cyclopenta[e]pyrazolo[1,5-a]pyrimidine-6-carboxamide; (6R,8R)-8-(1-(1,1-difluoroethyl)-1H-pyrazol-3-yl)-2-fluoro-8-methyl-N-(6-(pyrimidin-2-yl)-5-(trifluoromethyl)pyridin-3-yl)-7,8-dihydro-6H-cyclopenta[e]pyrazolo[1,5-a]pyrimidine-6-carboxamide

### Examples 166, 167: (6S,8R)-8-(1-(1,1-difluoroethyl)-1H-pyrazol-3-yl)-2-fluoro-8-methyl-N-(6-(pyrimidin-2-yl)-5-(trifluoromethyl)pyridin-3-yl)-7,8-dihydro-6H-cyclopenta[e]pyrazolo[1,5-a]pyrimidine-6-carboxamide; (6R,8S)-8-(1-(1,1-difluoroethyl)-1H-pyrazol-3-yl)-2-fluoro-8-methyl-N-(6-(pyrimidin-2-yl)-5-(trifluoromethyl)pyridin-3-yl)-7,8-dihydro-6H-cyclopenta[e]pyrazolo[1,5-a]pyrimidine-6-carboxamide

The relative and absolute stereochemistry of each example was not determined.

### Step 1: 2-methyl-2-(1H-pyrazol-3-yl)cyclopentan-1-one

In analogy to Method A33 step 1 but using 2-methyl-2-(1-((2-(trimethylsilyl)ethoxy)methyl)-1H-pyrazol-3-yl)cyclopentan-1-one (A9-4, 31.0 g, 105 mmol), the title compound was obtained as a yellow oil (9.0 g, 52% yield). LC-MS: m/z 165 [M+H]⁺.

### Step 2: 2-(1-(2-bromo-1,1-difluoroethyl)-1H-pyrazol-3-yl)-2-methylcyclopentan-1-one

To a stirred solution of 2-methyl-2-(1H-pyrazol-3-yl)cyclopentan-1-one (4.5 g, 27 mmol) in THE (200 mL) were added DBU (8.3 g, 54 mmol) and 2-bromo-1,1-difluoroethene (50.0 g, 349 mmol) at −40° C. The reaction mixture was warmed to 50° C. and stirred for 16 h. The reaction was quenched with water (100 mL). The resulting mixture was extracted with ethyl acetate (3×100 mL). The combined organic layers were washed with brine (100 ml), dried over anhydrous sodium sulfate and concentrated under reduced pressure. The residue was purified by reverse phase column to afford the title compound (2.3 g, 27% yield) as a yellow oil. LC-MS: m/z 307[M+H]⁺.

### Step 3: 2-(1-(1,1-difluoroethyl)-1H-pyrazol-3-yl)-2-methylcyclopentan-1-one

To a solution of 2-(1-(2-bromo-1.1-difluoroethyl)-1H-pyrazol-3-yl)-2-methylcyclopentan-1-one (4.5 g, 14 mmol) in EtOH (200 mL) were added Pd(OH)₂/C (4.1 g, 1 mmol, 10%) and Pd/C (3.5 g, 1 mmol, 10%). The mixture was degassed and then stirred at 120° C. for 48 h under hydrogen pressure [30 atm]. The solids were filtered out, and the filtrate was concentrated under reduced pressure. The residue was applied on a silica gel column and eluted with PE/EtOAc (10:1) to afford the title compound (1.8 g, 37% yield) as a yellow oil. ¹H NMR (400 MHz, Chloroform-d) δ 7.75 (d, J=2.8 Hz, 1H), 6.36 (d, J=2.8 Hz, 1H), 2.67-2.76 (m, 1H), 2.34-2.44 (m, 1H), 2.26 (t, J=16.0 Hz, 3H), 1.92-2.12 (m, 4H), 1.43 (s, 3H). LC-MS: m/z 229 [M+H]⁺.

### Step 4: 2-(1-(1,1-difluoroethyl)-1H-pyrazol-3-yl)-5-((dimethylamino)methylene)-2-methylcyclopentan-1-one

In analogy to Method A1 step 4 but using 2-(1-(1,1-difluoroethyl)-1H-pyrazol-3-yl)-2-methylcyclopentan-1-one (100 mg, 440 μmol) in 1-tert-butoxy-N,N,N′,N′-tetramethyl-methanediamine (382 mg, 2.2 mmol) and stirring at 100° C. for 1 h, the crude title compound was obtained as a red oil (100 mg). LC-MS: m/z 284 [M+H]⁺.

### Step 5: 8-(1-(1,1-difluoroethyl)-1H-pyrazol-3-yl)-2-fluoro-8-methyl-7,8-dihydro-6H-cyclopenta[e]pyrazolo[1,5-a]pyrimidine

In analogy to Method A1 step 5 but using 2-(1-(1,1-difluoroethyl)-1H-pyrazol-3-yl)-5-((dimethylamino)methylene)-2-methylcyclopentan-1-one (100 mg, 353 mol) and 3-fluoro-1H-pyrazol-5-amine (36 mg, 353 mol) in EtOH (5 mL) and HCl (0.5 mL, 4M in dioxane) and stirring at 100° C. for 1 h, the title compound was obtained as a yellow solid (50 mg, 44% yield). LC-MS: m/z 322 [M+H]⁺.

### Step 6: 8-(1-(1,1-difluoroethyl)-1H-pyrazol-3-yl)-2-fluoro-8-methyl-7,8-dihydro-6H-cyclopenta[e]pyrazolo[1,5-a]pyrimidine-6-carbonitrile

In analogy to Method A1 step 6 but using 8-(1-(1,1-difluoroethyl)-1H-pyrazol-3-yl)-2-fluoro-8-methyl-7,8-dihydro-6H-cyclopenta[e]pyrazolo[1,5-a]pyrimidine (400 mg, 1.2 mmol) in chlorobenzene (40 mL), the title compound was obtained as a yellow solid (200 mg, 23% yield). LC-MS: m/z 347 [M+H]⁺.

### Step 7: 8-(1-(1,1-difluoroethyl)-1H-pyrazol-3-yl)-2-fluoro-8-methyl-7,8-dihydro-6H-cyclopenta[e]pyrazolo[1,5-a]pyrimidine-6-carboxamide (A46-7)

In analogy to Method A33 step 7 but using 8-(1-(1,1-difluoroethyl)-1H-pyrazol-3-yl)-2-fluoro-8-methyl-7,8-dihydro-6H-cyclopenta[e]pyrazolo[1,5-a]pyrimidine-6-carbonitrile (65 mg, 187 mol), the title compound was obtained as a yellow oil (A46-7, 30 mg, 44% yield). LC-MS: m/z 365 [M+H]⁺.

### Step 8: 2-(5-bromo-3-(trifluoromethyl)pyridin-2-yl)pyrimidine

To a stirred mixture of 6-(pyrimidin-2-yl)-5-(trifluoromethyl)pyridin-3-amine (A38-2, 260 mg, 1.1 mmol) in HBr (2 mL, 33% in acetic acid) was added a solution of NaNO₂ (224 mg, 3.3 mmol) in water (2 mL) dropwise at 0° C., and it was stirred at 0° C. for 30 min. CuBr (202 mg, 1.4 mmol) was added at 0° C., and the reaction mixture was stirred at 0° C. for 1 h. The mixture was diluted with water (20 mL) and extracted with DCM (3×20 mL). The combined organic layers were dried over anhydrous sodium sulfate and concentrated under reduced pressure. The residue was applied on a silica gel column and eluted with PE/EtOAc (3:1) to afford the title compound (210 mg, 64% yield) as a yellow solid. LC-MS: m/z 304 [M+H]⁺.

### Step 9: (trans)-8-(1-(1,1-difluoroethyl)-1H-pyrazol-3-yl)-2-fluoro-8-methyl-N-(6-(pyrimidin-2-yl)-5-(trifluoromethyl)pyridin-3-yl)-7,8-dihydro-6H-cyclopenta[e]pyrazolo[1,5-a]pyrimidine-6-carboxamide and (cis)-8-(1-(1,1-difluoroethyl)-1H-pyrazol-3-yl)-2-fluoro-8-methyl-N-(6-(pyrimidin-2-yl)-5-(trifluoromethyl)pyridin-3-yl)-7,8-dihydro-6H-cyclopenta[e]pyrazolo[1,5-a]pyrimidine-6-carboxamide

In analogy to Method A33 step 8 but using 8-(1-(1,1-difluoroethyl)-1H-pyrazol-3-yl)-2-fluoro-8-methyl-7,8-dihydro-6H-cyclopenta[e]pyrazolo[1,5-a]pyrimidine-6-carboxamide (30 mg, 82 μmol) and 2-(5-bromo-3-(trifluoromethyl)pyridin-2-yl)pyrimidine (30 mg, 98 mol), racemic mixtures of the title compounds were obtained: trans isomer (12 mg, 21% yield) as a white solid, LC-MS: m/z 588 [M+H]⁺; cis-isomer (6 mg, 10% yield) as a white solid, LC-MS: m/z 588 [M+H]⁺.

### Step 10: Separation of Enantiomers to Obtain Example 164 and Example 165

25 mg of (trans)-8-(1-(1,1-difluoroethyl)-1H-pyrazol-3-yl)-2-fluoro-8-methyl-N-(6-(pyrimidin-2-yl)-5-(trifluoromethyl)pyridin-3-yl)-7,8-dihydro-6H-cyclopenta[e]pyrazolo[1,5-a]pyrimidine-6-carboxamide were submitted to chiral HPLC purification. (Column: CHIRALPAK IF, 2*25 cm, 5 m; Mobile Phase A: Hex (0.1% FA)-HPLC, Mobile Phase B: EtOH: DCM=1:1-HPLC; Flow rate: 20 ML/MIN mL/min; Gradient: isocratic 50% Mobile Phase B; Wave Length: 254/220 nm nm; RT1 (min): 5.47; RT2 (min): 10.61; Sample Solvent: EtOH-HPLC; Injection Volume: 1.3 mL; Number Of Runs: 2). The second eluting isomer was concentrated and lyophilized to afford Example 164 (5.4 mg, 21% yield) as an off-white solid. The first eluting isomer was concentrated and lyophilized to afford Example 165 (11.6 mg, 44% yield) as a light-yellow solid.

Example 164: ¹H NMR (400 MHz, DMSO-d₆) δ: 11.13 (s, 1H), 9.08 (d, J=2.4 Hz, 1H), 8.97 (d, J=4.8 Hz, 2H), 8.74 (s, 1H), 8.72 (d, J=2.4 Hz, 1H), 8.18 (d, J=2.8 Hz, 1H), 7.64 (t, J=4.8 Hz, 1H), 6.57-6.59 (m, 2H), 4.55 (t, J=8.4 Hz, 1H), 3.05-3.11 (m, 1H), 2.56-2.61 (m, 1H), 2.20 (t, J=16.4 Hz, 3H), 2.03 (s, 3H). LC-MS: m/z 588 [M+H]⁺.

Example 165: ¹H NMR (400 MHz, DMSO-d₆) δ: 11.10 (s, 1H), 9.08 (d, J=2.4 Hz, 1H), 8.97 (d, J=4.8 Hz, 2H), 8.74 (s, 1H), 8.72 (d, J=2.4 Hz, 1H), 8.18 (d, J=2.8 Hz, 1H), 7.64 (t, J=4.8 Hz, 1H), 6.57-6.59 (m, 2H), 4.55 (t, J=8.4 Hz, 1H), 3.05-3.11 (m, 1H), 2.56-2.61 (m, 1H), 2.21 (t, J=16.4 Hz, 3H), 2.03 (s, 3H). LC-MS: m/z 588 [M+H]⁺.

### Step 11: Separation of Enantiomers to Obtain Example 166 and Example 167

23 mg of (cis)-8-(1-(1,1-difluoroethyl)-1H-pyrazol-3-yl)-2-fluoro-8-methyl-N-(6-(pyrimidin-2-yl)-5-(trifluoromethyl)pyridin-3-yl)-7,8-dihydro-6H-cyclopenta[e]pyrazolo[1,5-a]pyrimidine-6-carboxamide were submitted to chiral HPLC purification. (Column: CHIRALPAK IG, 2*25 cm, 5 m; Mobile Phase A: Hex (0.1% FA)-HPLC, Mobile Phase B: EtOH: DCM=1:1-HPLC; Flow rate: 20 ML/MIN mL/min; Gradient: isocratic 40% Mobile Phase B; Wave Length: 254/220 nm nm; RT1 (min): 7.17; RT2 (min): 10.41; Sample Solvent: EtOH-HPLC; Injection Volume: 0.6 mL; Number Of Runs: 4). The second eluting isomer was concentrated and lyophilized to afford Example 166 (2.7 mg, 11% yield) as a white solid. The first eluting isomer was concentrated and lyophilized to afford Example 167 (1.3 mg, 5% yield) as a white solid.

Example 166: ¹H NMR (400 MHz, DMSO-d₆) δ: 11.00 (s, 1H), 9.05 (d, J=2.0 Hz, 1H), 8.96 (d, J=4.8 Hz, 2H), 8.73 (s, 1H), 8.67 (d, J=2.0 Hz, 1H), 8.11 (d, J=2.4 Hz, 1H), 7.63 (t, J=4.8 Hz, 1H), 6.57 (d, J=5.2 Hz, 1H), 6.48 (d, J=2.8 Hz, 1H), 4.56-4.60 (m, 1H), 2.99-3.03 (m, 1H), 2.78-2.85 (m, 1H), 2.09 (t, J=16.4 Hz, 3H), 1.93 (s, 3H). LC-MS: m/z 588 [M+H]⁺.

Example 167: ¹H NMR (400 MHz, DMSO-d₆) δ: 11.01 (s, 111), 9.05 (d, J=2.4 Hz, 111), 8.96 (d, J=4.8 Hz, 2H), 8.73 (s, 1H), 8.67 (d, J=2.0 Hz, 1H), 8.11 (d, J=2.4 Hz, 1H), 7.63 (t, J=4.8 Hz, 1H), 6.57 (d, J=4.8 Hz, 1H), 6.48 (d, J=2.8 Hz, 1H), 4.56-4.60 (m, 1H), 2.99-3.03 (m, 1H), 2.78-2.85 (m, 1H), 2.09 (t, J=16.4 Hz, 3H), 1.93 (s, 3H). LC-MS: m/z 588 [M+H]⁺.

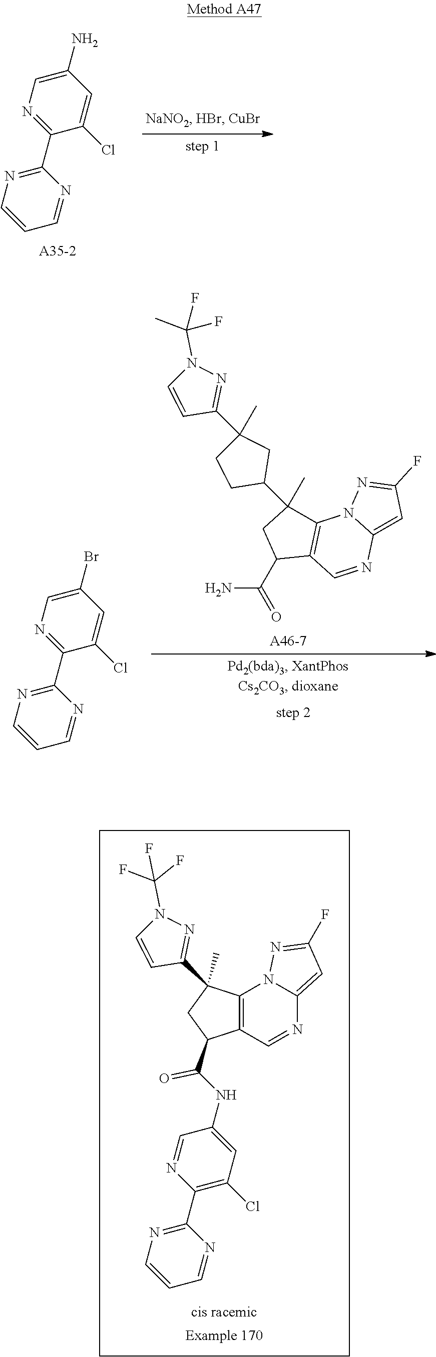 [SMILES: Nc1cnc(-c2ncccn2)c(Cl)c1 ; Clc1cc(Br)cnc1-c1ncccn1 ; CC1(c2ccn(C(C)(F)F)n2)CCC(C2(C)CC(C(N)=O)c3cnc4cc(F)nn4c32)C1 ; C[C@@]1(c2ccn(C(F)(F)F)n2)C[C@H](C(=O)Nc2cnc(-c3ncccn3)c(Cl)c2)c2cnc3cc(F)nn3c21]

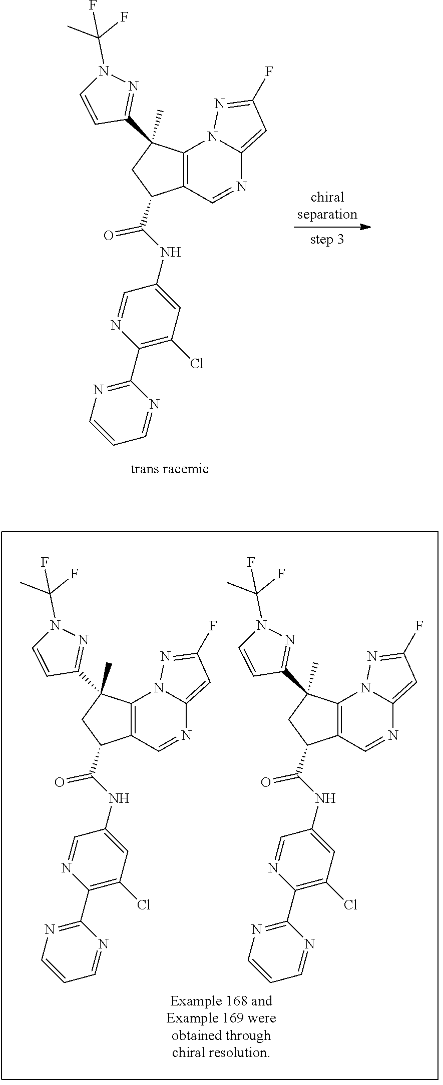

### Examples 168, 169: (6S,8S)—N-(5-chloro-6-(pyrimidin-2-yl)pyridin-3-yl)-8-(1-(1,1-difluoroethyl)-1H-pyrazol-3-yl)-2-fluoro-8-methyl-7,8-dihydro-6H-cyclopenta[e]pyrazolo[1,5-a]pyrimidine-6-carboxamide, (6R,8R)—N-(5-chloro-6-(pyrimidin-2-yl)pyridin-3-yl)-8-(1-(1,1-difluoroethyl)-1H-pyrazol-3-yl)-2-fluoro-8-methyl-7,8-dihydro-6H-cyclopenta[e]pyrazolo[1,5-a]pyrimidine-6-carboxamide

Example 170: (cis)-N-(5-chloro-6-(pyrimidin-2-yl)pyridin-3-yl)-8-(1-(1,1-difluoroethyl)-1H-pyrazol-3-yl)-2-fluoro-8-methyl-7,8-dihydro-6H-cyclopenta[e]pyrazolo[1,5-a]pyrimidine-6-carboxamide

The relative and absolute stereochemistry of each example was not determined.

### Step 1: 2-(5-bromo-3-chloropyridin-2-yl)pyrimidine

In analogy to Method A46 step 8 but using 5-chloro-6-(pyrimidin-2-yl)pyridin-3-amine (A35-2, 120 mg, 581 μmol), the title compound was obtained as a yellow solid (110 mg, 70% yield). LC-MS: m/z 270 [M+H]⁺.

### Step 2: (trans)-N-(5-chloro-6-(pyrimidin-2-yl)pyridin-3-yl)-8-(1-(1,1-difluoroethyl)-1H-pyrazol-3-yl)-2-fluoro-8-methyl-7,8-dihydro-6H-cyclopenta[e]pyrazolo[1,5-a]pyrimidine-6-carboxamide and Example 170

In analogy to Method A33 step 8 but using 8-(1-(1,1-difluoroethyl)-1H-pyrazol-3-yl)-2-fluoro-8-methyl-7,8-dihydro-6H-cyclopenta[e]pyrazolo[1,5-a]pyrimidine-6-carboxamide (A46-7, 30 mg, 82 μmol) and 2-(5-bromo-3-chloropyridin-2-yl)pyrimidine (26.7 mg, 98 μmol), racemic mixtures of the title compounds were obtained: trans isomer (1.3 mg, 2% yield) as a white solid, LC-MS: m/z 554 [M+H]⁺; Example 170 (1.2 mg, 2% yield) as a white solid.

Example 170: ¹H NMR (400 MHz, DMSO-d₆) δ:10.89 (s, 1H), 8.97 (d, J=4.8 Hz, 2H), 8.77 (d, J=2.0 Hz, 1H), 8.72 (s, 1H), 8.41 (d, J=2.0 Hz, 1H), 8.11 (d, J=2.8 Hz, 1H), 7.61 (t, J=4.8 Hz, 1H), 6.56 (d, J=4.8 Hz, 1H), 6.46 (d, J=2.8 Hz, 1H), 4.54-4.58 (m, 1H), 2.93-2.98 (m, 1H), 2.78-2.84 (m, 1H), 2.12 (t, J=16.4 Hz, 3H), 1.92 (s, 3H). LC-MS: m/z 554 [M+H]⁺.

### Step 3: Separation of Enantiomers to Obtain Example 168 and Example 169

5.8 mg of (trans)-N-(5-chloro-6-(pyrimidin-2-yl)pyridin-3-yl)-8-(1-(1,1-difluoroethyl)-1H-pyrazol-3-yl)-2-fluoro-8-methyl-7,8-dihydro-6H-cyclopenta[e]pyrazolo[1,5-a]pyrimidine-6-carboxamide were submitted to chiral HPLC purification. (Column: CHIRALPAK IG, 2*25 cm, 5 μm; Mobile Phase A: Hex (0.1% FA)-HPLC, Mobile Phase B: EtOH: DCM=1:1-HPLC; Flow rate: 20 ML/MIN mL/min; Gradient: isocratic 60% Mobile Phase B; Wave Length: 254/220 nm nm; RT1 (min): 6.138; RT2 (min): 12.152; Sample Solvent: EtOH-HPLC; Injection Volume: 1.5 mL; Number Of Runs: 1). The second eluting isomer was concentrated and lyophilized to afford Example 168 (1.8 mg, 31% yield) as an off-white solid. The first eluting isomer was concentrated and lyophilized to afford Example 169 (1.3 mg, 22% yield) as a white solid.

Example 168: ¹H NMR (400 MHz, DMSO-d₆) δ:10.97 (s, 1H), 8.98 (d, J=4.8 Hz, 2H), 8.80 (d, J=2.0 Hz, 1H), 8.72 (s, 1H), 8.46 (d, J=2.0 Hz, 1H), 8.18 (d, J=2.8 Hz, 1H), 7.61 (t, J=4.8 Hz, 1H), 6.56-6.59 (m, 2H), 4.51-4.55 (m, 1H), 3.05-3.10 (m, 1H), 2.56-2.67 (m, 1H), 2.20 (t, J=16.4 Hz, 3H), 2.03 (s, 3H). LC-MS: m/z 554 [M+H]⁺.

Example 169: ¹H NMR (400 MHz, DMSO-d₆) δ:10.97 (s, 1H), 8.98 (d, J=4.8 Hz, 2H), 8.80 (d, J=2.0 Hz, 1H), 8.72 (s, 1H), 8.46 (d, J=1.6 Hz, 1H), 8.18 (d, J=2.4 Hz, 1H), 7.62 (t, J=4.8 Hz, 1H), 6.56-6.59 (m, 2H), 4.51-4.55 (m, 1H), 3.04-3.10 (m, 1H), 2.56-2.67 (m, 1H), 2.20 (t, J=16.4 Hz, 3H), 2.03 (s, 3H). LC-MS: m/z 554 [M+H]⁺.

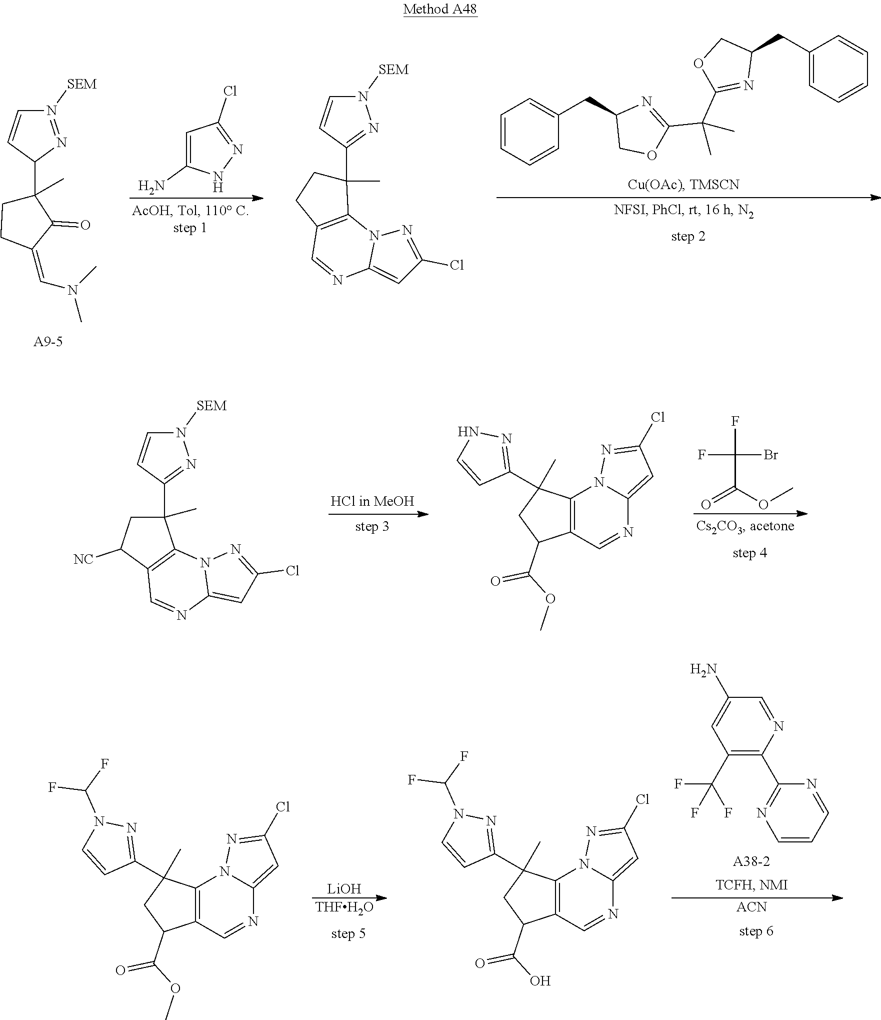

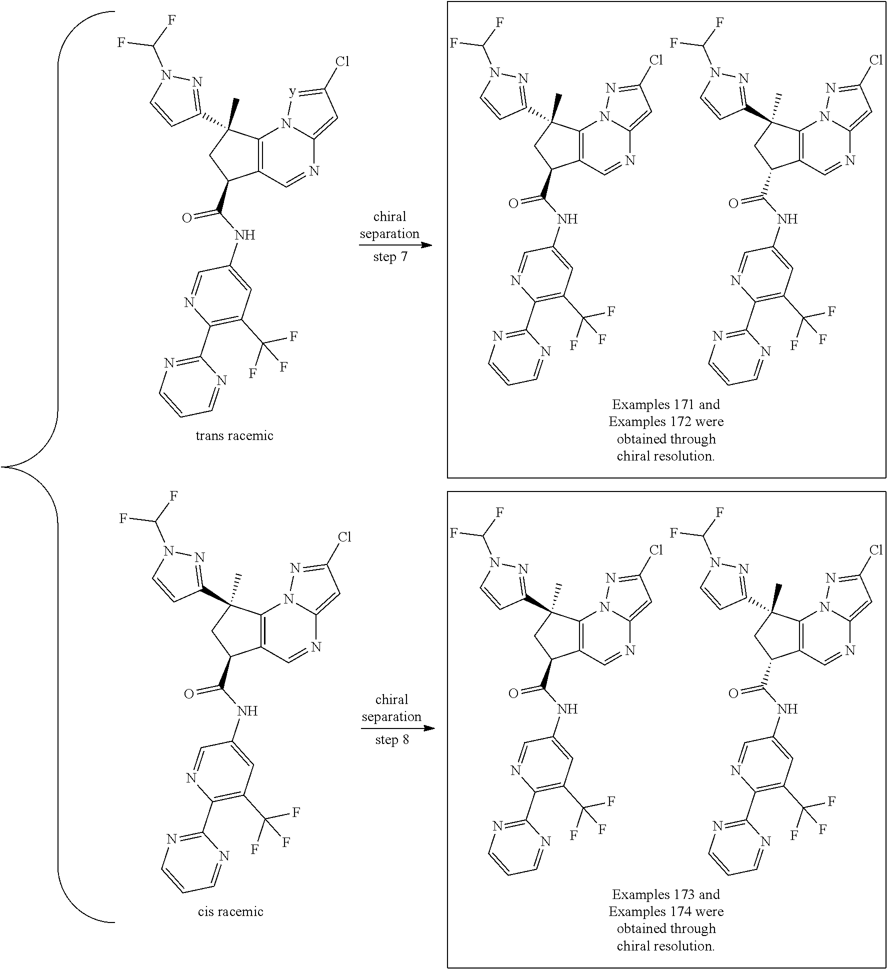

### Examples 171, 172: (6S,8S)-2-chloro-8-(1-(difluoromethyl)-1H-pyrazol-3-yl)-8-methyl-N-(6-(pyrimidin-2-yl)-5-(trifluoromethyl)pyridin-3-yl)-7,8-dihydro-6H-cyclopenta[e]pyrazolo[1,5-a]pyrimidine-6-carboxamide; (6R,8R)-2-chloro-8-(1-(difluoromethyl)-1H-pyrazol-3-yl)-8-methyl-N-(6-(pyrimidin-2-yl)-5-(trifluoromethyl)pyridin-3-yl)-7,8-dihydro-6H-cyclopenta[e]pyrazolo[1,5-a]pyrimidine-6-carboxamide

### Examples 173, 174: (6S,8R)-2-chloro-8-(1-(difluoromethyl)-1H-pyrazol-3-yl)-8-methyl-N-(6-(pyrimidin-2-yl)-5-(trifluoromethyl)pyridin-3-yl)-7,8-dihydro-6H-cyclopenta[e]pyrazolo[1,5-a]pyrimidine-6-carboxamide; (6R,8S)-2-chloro-8-(1-(difluoromethyl)-1H-pyrazol-3-yl)-8-methyl-N-(6-(pyrimidin-2-yl)-5-(trifluoromethyl)pyridin-3-yl)-7,8-dihydro-6H-cyclopenta[e]pyrazolo[1,5-a]pyrimidine-6-carboxamide

The relative and absolute stereochemistry of each example was not determined.

### Step 1: 2-chloro-8-methyl-8-(1-((2-(trimethylsilyl)ethoxy)methyl)-1H-pyrazol-3-yl)-7,8-dihydro-6H-cyclopenta[e]pyrazolo[1,5-a]pyrimidine

In analogy to Method A6 step 6 but using 5-((dimethylamino)methylene)-2-methyl-2-(1-((2-(trimethylsilyl)ethoxy)methyl)-1H-pyrazol-3-yl)cyclopentan-1-one (6 g, 17 mmol) and 3-chloro-1H-pyrazol-5-amine (2 g, 17 mmol), stirring at 110° C. for 1 h, the title compound was obtained (2.9 g, 41% yield) as a red solid. LC-MS: m/z 404 [M+H]⁺.

### Step 2: 2-chloro-8-methyl-8-(1-((2-(trimethylsilyl)ethoxy)methyl)-1H-pyrazol-3-yl)-7,8-dihydro-6H-cyclopenta[e]pyrazolo[1,5-a]pyrimidine-6-carbonitrile

In analogy to Method A6 step 7 but using 2-chloro-8-methyl-8-(1-((2-(trimethylsilyl)ethoxy)methyl)-1H-pyrazol-3-yl)-7,8-dihydro-6H-cyclopenta[e]pyrazolo[1,5-a]pyrimidine (1.1 g, 3 mmol) in chlorobenzene (20 mL), the title compound was obtained as a yellow oil (200 mg, 17% yield) as a yellow oil. LC-MS: m/z 429 [M+H]⁺.

### Step 3: Methyl 2-chloro-8-methyl-8-(1H-pyrazol-3-yl)-7,8-dihydro-6H-cyclopenta[e]pyrazolo[1,5-a]pyrimidine-6-carboxylate

A solution of 2-chloro-8-methyl-8-(1-((2-(trimethylsilyl)ethoxy)methyl)-1H-pyrazol-3-yl)-7,8-dihydro-6H-cyclopenta[e]pyrazolo[1,5-a]pyrimidine-6-carbonitrile (400 mg, 932 μmol) in HCl (10 mL, 4M in MeOH) was stirred at 80° C. for 2 h. The mixture was concentrated under reduced pressure to give the title compound (200 mg, 65% yield) as a yellow oil, which was used directly in the next step. LC-MS: m/z 332 [M+H]⁺.

### Step 4: Methyl 2-chloro-8-(1-(difluoromethyl)-1H-pyrazol-3-yl)-8-methyl-7,8-dihydro-6H-cyclopenta[e]pyrazolo[1,5-a]pyrimidine-6-carboxylate

In analogy to Method A23 step 1 but using methyl 2-chloro-8-methyl-8-(1H-pyrazol-3-yl)-7,8-dihydro-6H-cyclopenta[e]pyrazolo[1,5-a]pyrimidine-6-carboxylate (200 mg, 602 mol) and stirring at 60° C. for 16 h, the title compound was obtained as a yellow oil (200 mg, 87% yield). LC-MS: m/z 382 [M+H]⁺.

### Step 5: 2-chloro-8-(1-(difluoromethyl)-1H-pyrazol-3-yl)-8-methyl-7,8-dihydro-6H-cyclopenta[e]pyrazolo[1,5-a]pyrimidine-6-carboxylic acid

In analogy to Method A7 step 2 but using methyl 2-chloro-8-(1-(difluoromethyl)-1H-pyrazol-3-yl)-8-methyl-7,8-dihydro-6H-cyclopenta[e]pyrazolo[1,5-a]pyrimidine-6-carboxylate (200 mg, 524 μmol) in THF (10 mL) and purification of the product by reverse phase column chromatography, the title compound was obtained as a yellow oil (130 mg, 67% yield). LC-MS: m/z 368 [M+H]⁺.

### Step 6: (trans)-2-chloro-8-(1-(difluoromethyl)-1H-pyrazol-3-yl)-8-methyl-N-(6-(pyrimidin-2-yl)-5-(trifluoromethyl)pyridin-3-yl)-7,8-dihydro-6H-cyclopenta[e]pyrazolo[1,5-a]pyrimidine-6-carboxamide and (cis)-2-chloro-8-(1-(difluoromethyl)-1H-pyrazol-3-yl)-8-methyl-N-(6-(pyrimidin-2-yl)-5-(trifluoromethyl)pyridin-3-yl)-7,8-dihydro-6H-cyclopenta[e]pyrazolo[1,5-a]pyrimidine-6-carboxamide

In analogy to Method A1 step 8 but using 2-chloro-8-(1-(difluoromethyl)-1H-pyrazol-3-yl)-8-methyl-7,8-dihydro-6H-cyclopenta[e]pyrazolo[1,5-a]pyrimidine-6-carboxylic acid (120 mg, 326 mol) and 6-(pyrimidin-2-yl)-5-(trifluoromethyl)pyridin-3-amine (A38-2, 78 mg, 326 mol), racemic mixtures of the title compounds were obtained: trans isomer (24 mg, 25% yield) as a pink solid, LC-MS: m/z 590[M+H]⁺; cis-isomer (19 mg, 19% yield) as a white solid, LC-MS: m/z 590[M+H]⁺.

### Step 7: Separation of Enantiomers to Obtain Example 171 and Example 172

(trans)-2-chloro-8-(1-(difluoromethyl)-1H-pyrazol-3-yl)-8-methyl-N-(6-(pyrimidin-2-yl)-5-(trifluoromethyl)pyridin-3-yl)-7,8-dihydro-6H-cyclopenta[e]pyrazolo[1,5-a]pyrimidine-6-carboxamide (24 mg, 40 mol) was submitted to chiral HPLC purification (Column: CHIRALPAK IG, 2*25 cm, 5 μm; Mobile Phase A: Hex (0.1% FA)-HPLC, Mobile Phase B: EtOH: DCM=1:1-HPLC; Flow rate: 20 ML/MIN mL/min; Gradient: isocratic 50% Mobile Phase B; Wave Length: 254/220 nm nm; RT1 (min): 5.732; RT2 (min): 13.43; Sample Solvent: EtOH-HPLC; Injection Volume: 1.0 mL; Number Of Runs: 2). The second eluting isomer was concentrated and lyophilized to afford Example 171 (5 mg, 40% yield) as a pink solid. The first eluting isomer was concentrated and lyophilized to afford Example 172 (9 mg, 72% yield) as a yellow solid.

Example 171: ¹H NMR (400 MHz, DMSO-d₆) δ 11.13 (s, 1H), 9.07 (d, J=2.4 Hz, 1H), 8.98 (d, J=4.8 Hz, 2H), 8.76 (s, 1H), 8.72 (d, J=2.4 Hz, 1H), 8.22 (d, J=2.8 Hz, 1H), 7.71 (t, J=59.2 Hz, 1H), 7.64 (t, J=4.8 Hz, 1H), 6.97 (s, 1H), 6.63 (d, J=2.8 Hz, 1H), 4.51-4.56 (m, 1H), 3.01-3.08 (m, 1H), 2.55-2.63 (m, 1H), 2.07 (s, 3H). LC-MS: m/z 590[M+H]⁺.

Example 172: ¹H NMR (400 MHz, DMSO-d₆) δ 11.12 (s, 1H), 9.07 (d, J=2.0 Hz, 1H), 8.98 (d, J=4.8 Hz, 2H), 8.76 (s, 1H), 8.72 (d, J=2.4 Hz, 1H), 8.22 (d, J=2.8 Hz, 1H), 7.71 (t, J=59.2 Hz, 1H), 7.64 (t, J=4.8 Hz, 1H), 6.97 (s, 1H), 6.63 (d, J=2.8 Hz, 1H), 4.51-4.57 (m, 1H), 3.00-3.08 (m, 1H), 2.55-2.63 (m, 1H), 2.07 (s, 3H). LC-MS: m/z 590[M+H]⁺.

### Step 8: Separation of Enantiomers to Obtain Example 173 and Example 174

(cis)-2-chloro-8-(1-(difluoromethyl)-1H-pyrazol-3-yl)-8-methyl-N-(6-(pyrimidin-2-yl)-5-(trifluoromethyl)pyridin-3-yl)-7,8-dihydro-6H-cyclopenta[e]pyrazolo[1,5-a]pyrimidine-6-carboxamide (19 mg, 32 mol) was submitted to chiral HPLC purification (Column: CHIRALPAK IG, 2*25 cm, 5 μm; Mobile Phase A: Hex (0.1% FA)-HPLC, Mobile Phase B: EtOH: DCM=1:1-HPLC; Flow rate: 20 ML/MIN mL/min; Gradient: isocratic 30% Mobile Phase B; Wave Length: 254/220 nm nm; RT1 (min): 12.948; RT2 (min): 18.039; Sample Solvent: EtOH-HPLC; Injection Volume: 1.2 mL; Number Of Runs: 2). The second eluting isomer was concentrated and lyophilized to afford Example 173 (6 mg, 63% yield) as a white solid. The first eluting isomer was concentrated and lyophilized to afford Example 174 (5 mg, 8 μmol, 52% yield,) as a white solid.

Example 173: ¹H NMR (400 MHz, DMSO-d₆) δ 11.03 (s, 1H), 9.04 (d, J=2.0 Hz, 1H), 8.97 (d, J=4.8 Hz, 2H), 8.77 (s, 1H), 8.66 (d, J=2.0 Hz, 1H), 8.14 (d, J=2.8 Hz, 1H), 7.69 (t, J=59.2 Hz, 1H), 7.64 (t, J=4.8 Hz, 1H), 6.95 (s, 1H), 6.48 (d, J=2.8 Hz, 1H), 4.56-4.62 (m, 1H), 2.81-2.97 (m, 2H), 1.95 (s, 3H). LC-MS: m/z 590[M+H]⁺.

Example 174: ¹H NMR (400 MHz, DMSO-d₆) δ 11.02 (s, 1H), 9.04 (d, J=2.0 Hz, 1H), 8.97 (d, J=4.8 Hz, 2H), 8.77 (s, 1H), 8.66 (d, J=2.4 Hz, 1H), 8.14 (d, J=2.8 Hz, 1H), 7.70 (t, J=59.2 Hz, 1H), 7.64 (t, J=4.8 Hz, 1H), 6.95 (s, 1H), 6.48 (d, J=2.8 Hz, 1H), 4.57-4.62 (m, 1H), 2.81-2.95 (m, 2H), 1.95 (s, 3H). LC-MS: m/z 590[M+H]⁺.

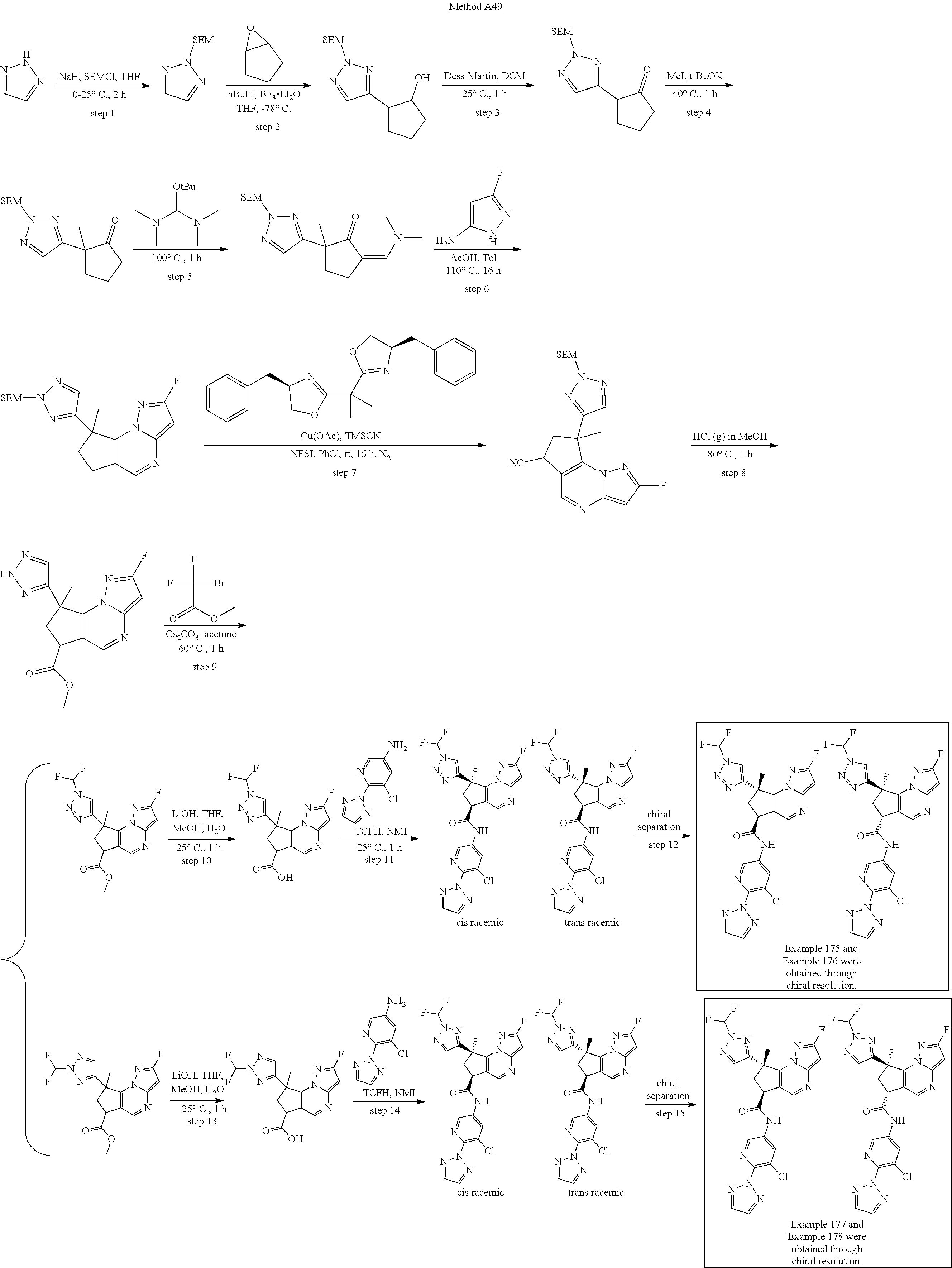

### Examples 175, 176: (6S,8S)—N-(5-chloro-6-(2H-1,2,3-triazol-2-yl)pyridin-3-yl)-8-(1-(difluoromethyl)-1H-1,2,3-triazol-4-yl)-2-fluoro-8-methyl-7,8-dihydro-6H-cyclopenta[e]pyrazolo[1,5-a]pyrimidine-6-carboxamide; (6R,8R)—N-(5-chloro-6-(2H-1,2,3-triazol-2-yl)pyridin-3-yl)-8-(1-(difluoromethyl)-11H-1,2,3-triazol-4-yl)-2-fluoro-8-methyl-7,8-dihydro-6H-cyclopenta[e]pyrazolo[1,5-a]pyrimidine-6-carboxamide

### Examples 177, 178: (6S,8S)—N-(5-chloro-6-(211-1,2,3-triazol-2-yl)pyridin-3-yl)-8-(2-(difluoromethyl)-2H-1,2,3-triazol-4-yl)-2-fluoro-8-methyl-7,8-dihydro-6H-cyclopenta[e]pyrazolo[1,5-a]pyrimidine-6-carboxamide; (6R,8R)—N-(5-chloro-6-(2H-1,2,3-triazol-2-yl)pyridin-3-yl)-8-(2-(difluoromethyl)-2H-1,2,3-triazol-4-yl)-2-fluoro-8-methyl-7,8-dihydro-6H-cyclopenta[e]pyrazolo[1,5-a]pyrimidine-6-carboxamide

The relative and absolute stereochemistry of each example was not determined.

### Step 1: 2-((2-(trimethylsilyl)ethoxy)methyl)-2H-1,2,3-triazole

In analogy to Method A6 step 1 but using 2H-triazole (30 g, 434.4 mmol), the title compound was obtained as a yellow oil (46 g, 45% yield). LC-MS: m/z 200 [M+H]⁺.

### Step 2: 2-(2-((2-(trimethylsilyl)ethoxy)methyl)-2H-1,2,3-triazol-4-yl)cyclopentan-1-ol

In analogy to Method A1 step 1 but using 2-((2-(trimethylsilyl)ethoxy)methyl)-2H-1,2,3-triazole (50 g, 250.9 mmol), the title compound was obtained as a yellow oil (33.5 g, 42% yield). LC-MS: m/z 284 [M+H]⁺.

### Step 3: 2-(2-((2-(trimethylsilyl)ethoxy)methyl)-2H-1,2,3-triazol-4-yl)cyclopentan-1-one

In analogy to Method A10 step 2 but using 2-(2-((2-(trimethylsilyl)ethoxy)methyl)-2H-1,2,3-triazol-4-yl)cyclopentan-1-ol (15 g, 52.9 mmol) and after purification by reverse phase column chromatography, the title compound was obtained as a yellow oil (15 g, 90% yield). LC-MS: m/z 282 [M+H]⁺.

### Step 4: 2-methyl-2-(2-((2-(trimethylsilyl)ethoxy)methyl)-2H-1,2,3-triazol-4-yl)cyclopentan-1-one

In analogy to Method A1 step 3 but using 2-(2-((2-(trimethylsilyl)ethoxy)methyl)-2H-1,2,3-triazol-4-yl)cyclopentan-1-one (10 g, 35.5 mmol), the title compound was obtained as a colorless oil (4.1 g, 36% yield). LC-MS: m/z 296 [M+H]⁺.

### Step 5: 5-((dimethylamino)methylene)-2-methyl-2-(2-((2-(trimethylsilyl)ethoxy)methyl)-2H-1,2,3-triazol-4-yl)cyclopentan-1-one

In analogy to Method A1 step 4 but using 2-methyl-2-(2-((2-(trimethylsilyl)ethoxy)methyl)-2H-1,2,3-triazol-4-yl)cyclopentan-1-one (3.9 g, 13.2 mmol) in 1-tert-butoxy-N,N,N′,N′-tetramethylmethanediamine (4.6 g, 26.4 mmol) and stirring at 100° C. for 1 h, the crude title compound was obtained as a brown oil (4.2 g) and was used in the next step without further purification. LC-MS: m/z 351 [M+H]⁺.

### Step 6: 2-fluoro-8-methyl-8-(2-((2-(trimethylsilyl)ethoxy)methyl)-2H-1,2,3-triazol-4-yl)-7,8-dihydro-6H-cyclopenta[e]pyrazolo[1,5-a]pyrimidine

In analogy to Method A1 step 5 but using 5-((dimethylamino)methylene)-2-methyl-2-(2-((2-(trimethylsilyl)ethoxy)methyl)-2H-1,2,3-triazol-4-yl)cyclopentan-1-one (1 g, 2.9 mmol) and after purification by reverse phase column chromatography, the title compound was obtained as a yellow oil (250 mg, 23% yield). LC-MS: m/z 389 [M+H]⁺.

### Step 7: 2-fluoro-8-methyl-8-(2-((2-(trimethylsilyl)ethoxy)methyl)-2H-1,2,3-triazol-4-yl)-7,8-dihydro-6H-cyclopenta[e]pyrazolo[1,5-a]pyrimidine-6-carbonitrile

In analogy to Method A6 step 7 but using 2-fluoro-8-methyl-8-(2-((2-(trimethylsilyl)ethoxy)methyl)-2H-1,2,3-triazol-4-yl)-7,8-dihydro-6H-cyclopenta[e]pyrazolo[1,5-a]pyrimidine (1 g, 2.6 mmol) in Chlorobenzene (20 mL), the title compound was obtained as a yellow oil (90 mg, 8% yield). LC-MS: m/z 414 [M+H]⁺.

### Step 8: Methyl 2-fluoro-8-methyl-8-(2H-1,2,3-triazol-4-yl)-7,8-dihydro-6H-cyclopenta[e]pyrazolo[1,5-a]pyrimidine-6-carboxylate

In analogy to Method A48 step 3 but using 2-fluoro-8-methyl-8-(2-((2-(trimethylsilyl)ethoxy)methyl)-2H-1,2,3-triazol-4-yl)-7,8-dihydro-6H-cyclopenta[e]pyrazolo[1,5-a]pyrimidine-6-carbonitrile (90 mg, 185 μmol), the title compound was obtained as a yellow oil (50 mg, 85% yield). LC-MS: m/z 317 [M+H]⁺.

### Step 9: Methyl 8-(1-(difluoromethyl)-1H-1,2,3-triazol-4-yl)-2-fluoro-8-methyl-7,8-dihydro-6H-cyclopenta[e]pyrazolo[1,5-a]pyrimidine-6-carboxylate and Methyl 8-(2-(difluoromethyl)-2H-1,2,3-triazol-4-yl)-2-fluoro-8-methyl-7,8-dihydro-6H-cyclopenta[e]pyrazolo[1,5-a]pyrimidine-6-carboxylate

In analogy to Method A23 step 1 but using 2-fluoro-8-methyl-8-(2H-1,2,3-triazol-4-yl)-7,8-dihydro-6H-cyclopenta[e]pyrazolo[1,5-a]pyrimidine-6-carboxylate (50 mg, 158 mol) and stirring at 60° C. for 1 h and after purification by prep-HPLC methyl 8-(1-(difluoromethyl)-1H-1,2,3-triazol-4-yl)-2-fluoro-8-methyl-7,8-dihydro-6H-cyclopenta[e]pyrazolo[1,5-a]pyrimidine-6-carboxylate (35 mg, 60% yield), LC-MS: m/z 367 [M+H]⁺ and methyl 8-(2-(difluoromethyl)-2H-1,2,3-triazol-4-yl)-2-fluoro-8-methyl-7,8-dihydro-6H-cyclopenta[e]pyrazolo[1,5-a]pyrimidine-6-carboxylate (20 mg, 35% yield), LC-MS: m/z 367 [M+H]⁺ were both obtained as white solids.

### Step 10: 8-(1-(difluoromethyl)-1H-1,2,3-triazol-4-yl)-2-fluoro-8-methyl-7,8-dihydro-6H-cyclopenta[e]pyrazolo[1,5-a]pyrimidine-6-carboxylic acid

In analogy to Method A7 step 2 but using 8-(1-(difluoromethyl)-1H-1,2,3-triazol-4-yl)-2-fluoro-8-methyl-7,8-dihydro-6H-cyclopenta[e]pyrazolo[1,5-a]pyrimidine-6-carboxylate (33 mg, 90.1 μmol), the crude title compound was obtained as a yellow oil (28 mg, 88% yield). LC-MS: m/z 353 [M+H]⁺.

### Step 11: (trans)-N-(5-chloro-6-(2H-1,2,3-triazol-2-yl)pyridin-3-yl)-8-(1-(difluoromethyl)-1H-1,2,3-triazol-4-yl)-2-fluoro-8-methyl-7,8-dihydro-6H-cyclopenta[e]pyrazolo[1,5-a]pyrimidine-6-carboxamide and (cis)-N-(5-chloro-6-(2H-1,2,3-triazol-2-yl)pyridin-3-yl)-8-(1-(difluoromethyl)-1H-1,2,3-triazol-4-yl)-2-fluoro-8-methyl-7,8-dihydro-6H-cyclopenta[e]pyrazolo[1,5-a]pyrimidine-6-carboxamide

In analogy to Method A1 step 8 but using 8-(1-(difluoromethyl)-1H-1,2,3-triazol-4-yl)-2-fluoro-8-methyl-7,8-dihydro-6H-cyclopenta[e]pyrazolo[1,5-a]pyrimidine-6-carboxylic acid (26 mg, 74 mol), racemic mixtures of the title compounds were obtained: trans isomer (10.4 mg, 26% yield) as a white solid; cis-isomer (5.6 mg, 14% yield) as a white solid.

Cis isomer: ¹H NMR (400 MHz, DMSO-d₆) δ 11.08 (s, 1H), 8.79 (s, 1H), 8.77 (s, 1H), 8.72 (d, J=2.0 Hz, 1H), 8.57 (d, J=2.0 Hz, 1H), 8.25 (t, J=58.0 Hz, 1H), 8.18 (s, 2H), 6.58 (d, J=4.8 Hz, 1H), 4.61 (t, J=8.0 Hz, 1H), 2.82-2.97 (m, 2H), 1.95 (s, 3H). LC-MS: m/z 530 [M+H]⁺.

### Step 12: Separation of Enantiomers to Obtain Example 175 and Example 176

9 mg of (trans)-N-(5-chloro-6-(2H-1,2,3-triazol-2-yl)pyridin-3-yl)-8-(1-(difluoromethyl)-1H-1,2,3-triazol-4-yl)-2-fluoro-8-methyl-7,8-dihydro-6H-cyclopenta[e]pyrazolo[1,5-a]pyrimidine-6-carboxamide (9 mg, 17 mol) were submitted to chiral HPLC purification (Column: CHIRALPAK IF, 2*25 cm, 5 μm; Mobile Phase A: Hex (0.1% FA)-HPLC, Mobile Phase B: EtOH: DCM=1:1-HPLC; Flow rate: 20 ML/MIN mL/min; Gradient: isocratic 40% Mobile Phase B; Wave Length: 254/220 nm nm; RT1 (min): 9.704; RT2 (min): 12.258; Sample Solvent: EtOH-HPLC; Injection Volume: 0.6 mL; Number Of Runs: 3). The second eluting isomer was concentrated and lyophilized to afford Example 175 (2.8 mg, 31% yield) as an off-white solid. The first eluting isomer was concentrated and lyophilized to afford Example 176 (3.9 mg, 43% yield) as an off-white solid.

Example 175: ¹H NMR (400 MHz, DMSO-d₆) δ 11.27 (s, 1H), 8.88 (s, 1H), 8.78 (s, 1H), 8.76 (d, J=2.4 Hz, 1H), 8.61 (d, J=2.4 Hz, 1H), 8.23 (t, J=58.0, 1H), 8.18 (s, 2H), 6.60 (d, J=4.8 Hz, 1H), 4.68 (t, J=8.0 Hz, 1H), 3.01-3.09 (m, 1H), 2.64-2.69 (m, 1H), 2.07 (s, 3H). LC-MS: m/z 530 [M+H]⁺.

Example 176: ¹H NMR (400 MHz, DMSO-d₆) δ 11.20 (s, 1H), 8.88 (s, 1H), 8.77 (s, 1H), 8.75 (d, J=2.4 Hz, 1H), 8.61 (d, J=2.4 Hz, 1H), 8.23 (t, J=58.0, 1H), 8.18 (s, 2H), 6.60 (d, J=4.8 Hz, 1H), 4.67 (t, J=8.0 Hz, 1H), 3.00-3.10 (m, 1H), 2.61-2.69 (m, 1H), 2.07 (s, 3H). LC-MS: m/z 530 [M+H]⁺.

### Step 13: 8-(2-(difluoromethyl)-2H-1,2,3-triazol-4-yl)-2-fluoro-8-methyl-7,8-dihydro-6H-cyclopenta[e]pyrazolo[1,5-a]pyrimidine-6-carboxylic acid

In analogy to Method A7 step 2 but using methyl 8-(2-(difluoromethyl)-2H-1,2,3-triazol-4-yl)-2-fluoro-8-methyl-7,8-dihydro-6H-cyclopenta[e]pyrazolo[1,5-a]pyrimidine-6-carboxylate (30 mg, 82 mol), the crude title compound was obtained as a yellow oil (20 mg, 69% yield). LC-MS (ES, m/z): 353[M+H]⁺.

### Step 14: (trans)-N-(5-chloro-6-(2H-1,2,3-triazol-2-yl)pyridin-3-yl)-8-(2-(difluoromethyl)-2H-1,2,3-triazol-4-yl)-2-fluoro-8-methyl-7,8-dihydro-6H-cyclopenta[e]pyrazolo[1,5-a]pyrimidine-6-carboxamide and (cis)-N-(5-chloro-6-(2H-1,2,3-triazol-2-yl)pyridin-3-yl)-8-(2-(difluoromethyl)-2H-1,2,3-triazol-4-yl)-2-fluoro-8-methyl-7,8-dihydro-6H-cyclopenta[e]pyrazolo[1,5-a]pyrimidine-6-carboxamide

In analogy to Method A1 step 8 but using 8-(2-(difluoromethyl)-2H-1,2,3-triazol-4-yl)-2-fluoro-8-methyl-7,8-dihydro-6H-cyclopenta[c]pyrazolo[1,5-a]pyrimidine-6-carboxylic acid (15 mg, 43 μmol), racemic mixtures of the title compounds were obtained: trans isomer (7 mg, 31% yield) as a white solid; cis-isomer (3 mg, 13% yield) as a white solid.

Cis isomer: ¹H NMR (400 MHz, DMSO-d₆) δ 11.15 (br, 1H), 8.79 (s, 1H), 8.72 (d, J=1.6 Hz, 1H), 8.56 (d, J=1.6 Hz, 1H), 8.23 (s, 1H), 8.18 (s, 2H), 8.07 (t, J=57.6 Hz, 1H), 6.60 (d, J=4.8 Hz, 1H), 4.63 (t, J=7.6 Hz, 1H), 2.83-2.93 (m, 2H), 1.97 (s, 3H). LC-MS: m/z 530 [M+H]⁺.

### Step 15: Separation of Enantiomers to Obtain Example 177 and Example 178

(trans)-N-(5-chloro-6-(2H-1,2,3-triazol-2-yl)pyridin-3-yl)-8-(2-(difluoromethyl)-2H-1,2,3-triazol-4-yl)-2-fluoro-8-methyl-7,8-dihydro-6H-cyclopenta[e]pyrazolo[1,5-a]pyrimidine-6-carboxamide (7 mg, 13 μmol) was submitted to chiral HPLC purification (Column: CHIRALPAK IG, 2*25 cm, 5 m; Mobile Phase A: Hex (0.1% FA)-HPLC, Mobile Phase B: EtOH: DCM=1:1-HPLC; Flow rate: 20 ML/MIN mL/min; Gradient: isocratic 50% Mobile Phase B; Wave Length: 254/220 nm nm; RT1 (min): 6.412; RT2 (min): 10.459; Injection Volume: 2 mL; Number Of Runs: 1). The second eluting isomer was concentrated and lyophilized to afford Example 177 (2.2 mg, 31% yield) as a white solid. The first eluting isomer was concentrated and lyophilized to afford Example 178 (2.8 mg, 37% yield) as a white solid.

Example 177: ¹H NMR (400 MHz, DMSO-d₆) δ 11.17 (s, 1H), 8.78 (s, 1H), 8.74 (d, J=2.0 Hz, 1H), 8.60 (d, J=2.0 Hz, 1H), 8.33 (s, 1H), 8.18 (s, 2H), 8.07 (t, J=57.2 Hz, 1H), 6.61 (d, J=4.8 Hz, 1H), 4.54-4.63 (m, 1H), 2.97-3.06 (m, 1H), 2.66-2.72 (m, 1H), 2.09 (s, 3H). LC-MS (ES, m/z): 530[M+H]⁺.

Example 178: ¹H NMR (400 MHz, DMSO-d₆) δ 11.17 (s, 1H), 8.78 (s, 1H), 8.74 (d, J=2.0 Hz, 1H), 8.60 (d, J=2.0 Hz, 1H), 8.33 (s, 1H), 8.18 (s, 2H), 8.07 (t, J=57.2 Hz, 1H), 6.61 (d, J=5.2 Hz, 1H), 4.51-4.64 (m, 1H), 2.97-3.10 (m, 1H), 2.66-2.72 (m, 1H), 2.09 (s, 3H). LC-MS (ES, m/z): 530[M+H]⁺.

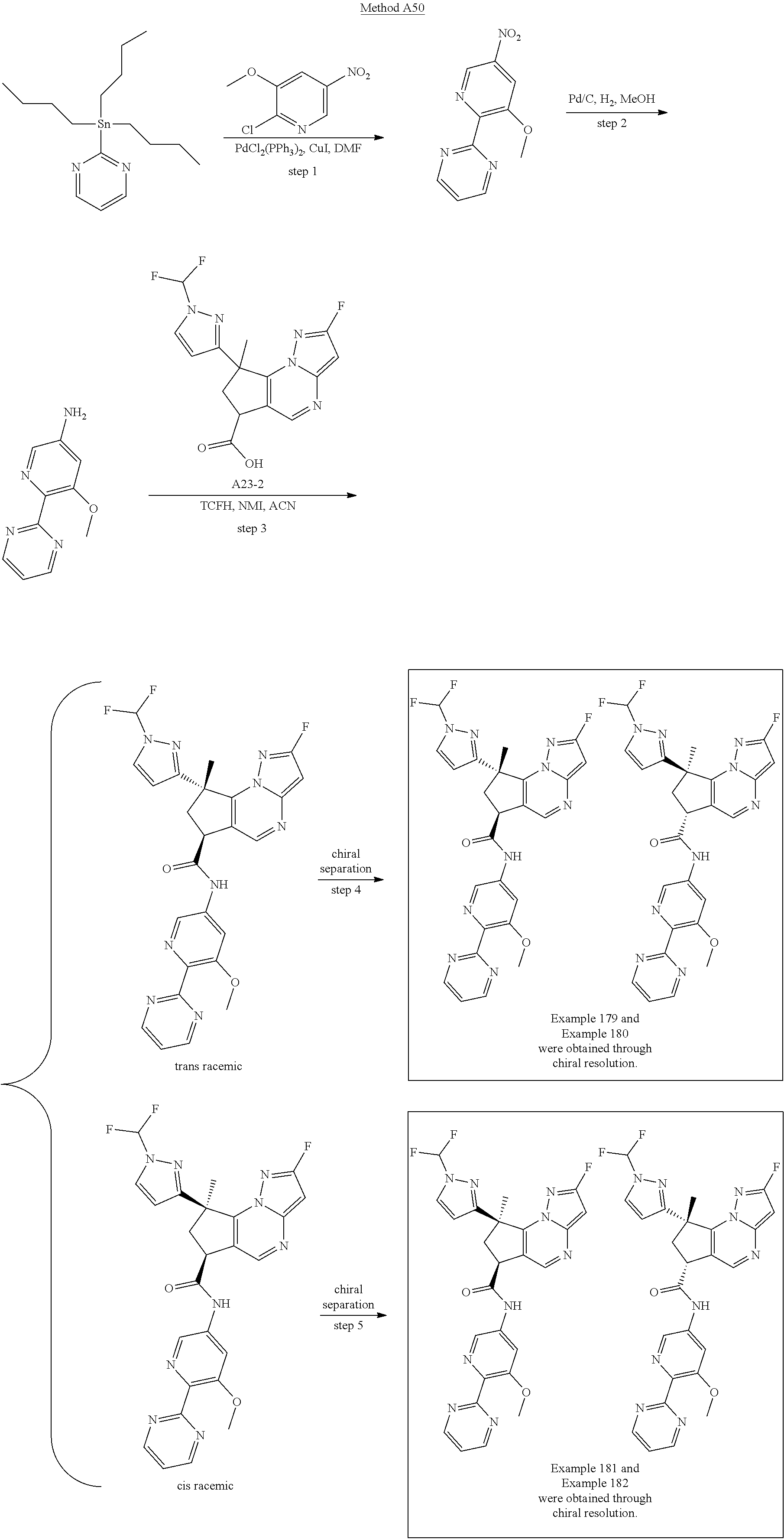

### Example 179, 180: (6S,8S)-8-(1-(difluoromethyl)-1H-pyrazol-3-yl)-2-fluoro-N-(5-methoxy-6-(pyrimidin-2-yl)pyridin-3-yl)-8-methyl-7,8-dihydro-6H-cyclopenta[e]pyrazolo[1,5-a]pyrimidine-6-carboxamide; (6R,8R)-8-(1-(difluoromethyl)-1H-pyrazol-3-yl)-2-fluoro-N-(5-methoxy-6-(pyrimidin-2-yl)pyridin-3-yl)-8-methyl-7,8-dihydro-6H-cyclopenta[e]pyrazolo[1,5-a]pyrimidine-6-carboxamide

### Examples 181, 182: (6S,8R)-8-(1-(difluoromethyl)-1H-pyrazol-3-yl)-2-fluoro-N-(5-methoxy-6-(pyrimidin-2-yl)pyridin-3-yl)-8-methyl-7,8-dihydro-6H-cyclopenta[e]pyrazolo[1,5-a]pyrimidine-6-carboxamide; (6R,8S)-8-(1-(difluoromethyl)-1H-pyrazol-3-yl)-2-fluoro-N-(5-methoxy-6-(pyrimidin-2-yl)pyridin-3-yl)-8-methyl-7,8-dihydro-6H-cyclopenta[e]pyrazolo[1,5-a]pyrimidine-6-carboxamide

The relative and absolute stereochemistry of each example was not determined.

### Step 1: 2-(3-methoxy-5-nitropyridin-2-yl)pyrimidine

In analogy to Method A35 step 1 but using 2-chloro-3-methoxy-5-nitro-pyridine (1 g, 5.3 mmol), the title compound was obtained as a yellow solid (520 mg, 42% yield). LC-MS: m/z 233 [M+H]⁺.

### Step 2: 5-methoxy-6-(pyrimidin-2-yl)pyridin-3-amine

In analogy to Method A15 step 2 but using 2-(3-methoxy-5-nitropyridin-2-yl)pyrimidine (150 mg, 581 μmol), the title compound was obtained as a yellow oil (83 mg, 71% yield). LC-MS: m/z 203 [M+H]⁺.

### Step 3: (trans)-8-(1-(difluoromethyl)-1H-pyrazol-3-yl)-2-fluoro-N-(5-methoxy-6-(pyrimidin-2-yl)pyridin-3-yl)-8-methyl-7,8-dihydro-6H-cyclopenta[e]pyrazolo[1,5-a]pyrimidine-6-carboxamide and (cis)-8-(1-(difluoromethyl)-1H-pyrazol-3-yl)-2-fluoro-N-(5-methoxy-6-(pyrimidin-2-yl)pyridin-3-yl)-8-methyl-7,8-dihydro-6H-cyclopenta[e]pyrazolo[1,5-a]pyrimidine-6-carboxamide

In analogy to Method A1 step 8 but using 8-(1-(difluoromethyl)-1H-pyrazol-3-yl)-2-fluoro-8-methyl-7,8-dihydro-6H-cyclopenta[e]pyrazolo[1,5-a]pyrimidine-6-carboxylic acid (55 mg, 157 μmol) and 5-methoxy-6-(pyrimidin-2-yl)pyridin-3-amine (38 mg, 188 μmol), racemic mixtures of the title compounds were obtained: trans isomer (23 mg, 26% yield) as an off-white solid, LC-MS: m/z 536 [M+H]⁺; cis-isomer (15 mg, 17% yield) as an off white solid, LC-MS: m/z 536 [M+H]⁺.

### Step 4: Separation of Enantiomers to Obtain Example 179 and Example 180

23 mg of (trans)-8-(1-(difluoromethyl)-1H-pyrazol-3-yl)-2-fluoro-N-(5-methoxy-6-(pyrimidin-2-yl)pyridin-3-yl)-8-methyl-7,8-dihydro-6H-cyclopenta[e]pyrazolo[1,5-a]pyrimidine-6-carboxamide were submitted to chiral HPLC purification (Column: CHIRALPAK IG, 2*25 cm, 5 μm; Mobile Phase A: Hex (0.1% FA)-HPLC, Mobile Phase B: EtOH: DCM=1:1-HPLC; Flow rate: 20 ML/MIN mL/min; Gradient: isocratic 70% Mobile Phase B; Wave Length: 254/220 nm nm; RT1 (min): 5.344; RT2 (min): 10.928; Sample Solvent: EtOH-HPLC; Injection Volume: 2.0 mL; Number Of Runs: 1). The first eluting isomer was concentrated and lyophilized to afford Example 179 (6.1 mg, 26% yield) as an off white solid. The second eluting isomer was concentrated and lyophilized to afford Example 180 (4.8 mg, 21% yield) as an off white solid.

Example 179: ¹HNMR (400 MHz, CDCL3) δ: 8.94 (d, J=4.8 Hz, 2H), 8.73 (br, 1H), 8.60 (s, 1H), 8.57 (s, 1H), 8.46 (s, 1H), 7.74 (d, J=2.8 Hz, 1H), 7.33 (d, J=4.8 Hz, 1H), 7.08 (t, J=60.8 Hz, 111), 6.75 (d, J=2.8 Hz, 111), 6.27 (d, J=4.8 Hz, 1H), 4.60 (t, J=8.0 Hz, 1H), 4.00 (s, 3H), 3.37-3.47 (m, 1H), 2.59-2.69 (m, 1H), 2.16 (s, 3H). LC-MS: m/z 536 [M+H]⁺.

Example 180: ¹HNMR (400 MHz, CDCL3) δ: 8.94 (d, J=4.8 Hz, 2H), 8.65 (br, 1H), 8.60 (s, 1H), 8.55 (s, 1H), 8.44 (s, 1H), 7.74 (d, J=2.4 Hz, 1H), 7.33 (d, J=4.8 Hz, 1H), 7.08 (t, J=60.8 Hz, 1H), 6.75 (d, J=2.8 Hz, 1H), 6.28 (d, J=4.8 Hz, 1H), 4.59 (t, J=8.0 Hz, 1H), 4.00 (s, 3H), 3.38-3.47 (m, 1H), 2.60-2.66 (m, 1H), 2.16 (s, 3H). LC-MS: m/z 536 [M+H]⁺.

### Step 5: Separation of Enantiomers to Obtain Example 181 and Example 182

15 mg of (cis)-8-(1-(difluoromethyl)-1H-pyrazol-3-yl)-2-fluoro-N-(5-methoxy-6-(pyrimidin-2-yl)pyridin-3-yl)-8-methyl-7,8-dihydro-6H-cyclopenta[c]pyrazolo[1,5-a]pyrimidine-6-carboxamide was submitted to chiral HPLC purification (Column: CHIRAL ART Cellulose-SC, 2*25 cm, 5 m; Mobile Phase A: Hex-HPLC, Mobile Phase B: IPA: DCM=1:1-HPLC; Flow rate: 20 ML/MIN mL/min; Gradient: isocratic 80% Mobile Phase B; Wave Length: 254/220 nm nm; RT1 (min): 7.973; RT2 (min): 11.305; Sample Solvent: EtOH-HPLC; Injection Volume: 1.0 mL; Number Of Runs: 2). The first eluting isomer was concentrated and lyophilized to afford Example 181 (3.5 mg, 24% yield) as a light pink solid. The second eluting isomer was concentrated and lyophilized to afford Example 182 (2.6 mg, 18% yield) as an off white solid.

Example 181: ¹HNMR (400 MHz, CDCL3) δ: 9.47 (s, 1H), 8.94 (d, J=4.8 Hz, 2H), 8.68 (s, 1H), 8.32 (s, 1H), 8.27 (s, 1H), 7.81 (d, J=2.8 Hz, 1H), 7.32 (d, J=4.8 Hz, 1H), 7.02 (t, J=60.4 Hz, 1H), 6.92 (d, J=2.8 Hz, 1H), 6.30 (d, J=5.1 Hz, 1H), 4.52-4.60 (m, 1H), 3.97 (s, 3H), 3.35-3.45 (m, 1H), 2.84-2.94 (m, 1H), 2.12 (s, 3H). LC-MS: m/z 536 [M+H]⁺.

Example 182: ¹HNMR (400 MHz, CDCL3) δ: 9.86 (s, 1H), 8.95 (d, J=4.8 Hz, 2H), 8.68 (s, 1H), 8.42-8.46 (m, 2H), 7.80 (d, J=2.8 Hz, 1H), 7.33 (d, J=4.8 Hz, 1H), 7.03 (t, J=60.4 Hz, 1H), 6.88 (d, J=2.8 Hz, 1H), 6.29 (d, J=5.1 Hz, 1H), 4.58-4.65 (m, 1H), 3.99 (s, 3H), 3.35-3.45 (m, 1H), 2.85-2.96 (m, 1H), 2.11 (s, 3H). LC-MS: m/z 536 [M+H]⁺.

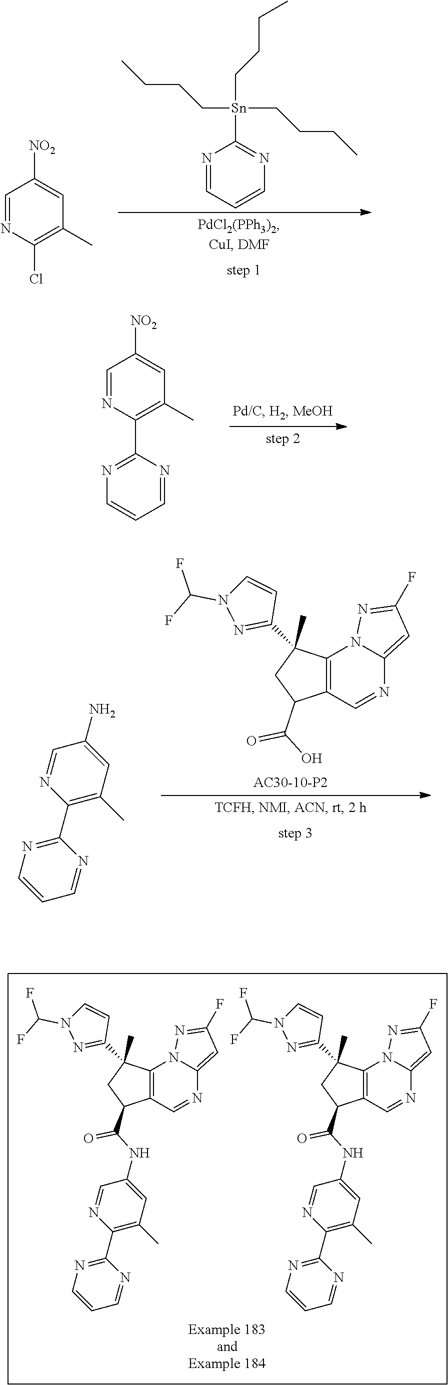

### Examples 183, Example 184: (6S,8S)-8-(1-(difluoromethyl)-1H-pyrazol-3-yl)-2-fluoro-8-methyl-N-(5-methyl-6-(pyrimidin-2-yl)pyridin-3-yl)-7,8-dihydro-6H-cyclopenta[e]pyrazolo[1,5-a]pyrimidine-6-carboxamide; (6R,8S)-8-(1-(difluoromethyl)-1H-pyrazol-3-yl)-2-fluoro-8-methyl-N-(5-methyl-6-(pyrimidin-2-yl)pyridin-3-yl)-7,8-dihydro-6H-cyclopenta[e]pyrazolo[1,5-a]pyrimidine-6-carboxamide

The relative and absolute stereochemistry of each example was not determined.

### Step 1: 2-(3-methyl-5-nitropyridin-2-yl)pyrimidine

In analogy to Method A30 step 12 but using 2-(tributylstannyl)pyrimidine (2.6 g, 6.9 mmol) and 2-chloro-3-methyl-5-nitropyridine (1 g, 5.8 mmol) and stirring at 120° C. for 1 h, the title compound was obtained as a white solid (600 mg, 45% yield). LC-MS: m/z 217 [M+H]⁺.

### Step 2: 5-methyl-6-(pyrimidin-2-yl)pyridin-3-amine

In analogy to Method A15 step 2 but using 2-(3-methyl-5-nitropyridin-2-yl)pyrimidine (500 mg, 2.3 mmol) and without further purification, the crude title compound was obtained as a brown solid (435 mg). LC-MS: m/z 187 [M+H]⁺.

### Step 3: Example 183 and Example 184

In analogy to Method A1 step 8 but using (8S)-8-(1-(difluoromethyl)-1H-pyrazol-3-yl)-2-fluoro-8-methyl-7,8-dihydro-6H-cyclopenta[e]pyrazolo[1,5-a]pyrimidine-6-carboxylic acid (A30-10-P2, 50 mg, 142 μmol) and 5-methyl-6-(pyrimidin-2-yl)pyridin-3-amine (32 mg, 169.7 mol), the diastereomeric title compounds were obtained: trans isomer Example 183 (16.5 mg, 41% yield) as a light pink solid and cis-isomer Example 184 (10.2 mg, 24% yield) as a light pink solid.

Example 183: ¹HNMR (400 MHz, CDCL3) δ:9.00 (br, 1H), 8.93 (d, J=4.8 Hz, 2H), 8.90 (s, 1H), 8.62 (s, 1H), 8.60 (s, 1H), 7.73 (d, J=2.8 Hz, 1H), 7.34 (t, J=4.8 Hz, 1H), 7.08 (t, J=60.8 Hz, 1H), 6.73 (d, J=2.4 Hz, 1H), 6.26 (d, J=4.8 Hz, 1H), 4.63 (t, J=8.0 Hz, 1H), 3.35-3.45 (m, 1H), 2.71 (s, 3H), 2.63-2.68 (m, 1H), 2.16 (s, 3H). LC-MS: m/z 520 [M+H]⁺.

Example 184: ¹HNMR (400 MHz, CDCL3) δ: 9.38 (s, 1H), 8.93 (d, J=4.8 Hz, 2H), 8.68 (s, 1H), 8.65 (s, 1H), 8.21 (s, 1H), 7.81 (d, J=2.4 Hz, 1H), 7.33 (t, J=4.8 Hz, 1H), 7.01 (t, J=60.4 Hz, 1H), 6.92 (d, J=2.4 Hz, 1H), 6.30 (d, J=4.8 Hz, 1H), 4.54-4.58 (m, 1H), 3.34-3.42 (m, 1H), 2.84-2.92 (m, 1H), 2.62 (s, 3H), 2.12 (s, 3H). LC-MS: m/z 520 [M+H]⁺.

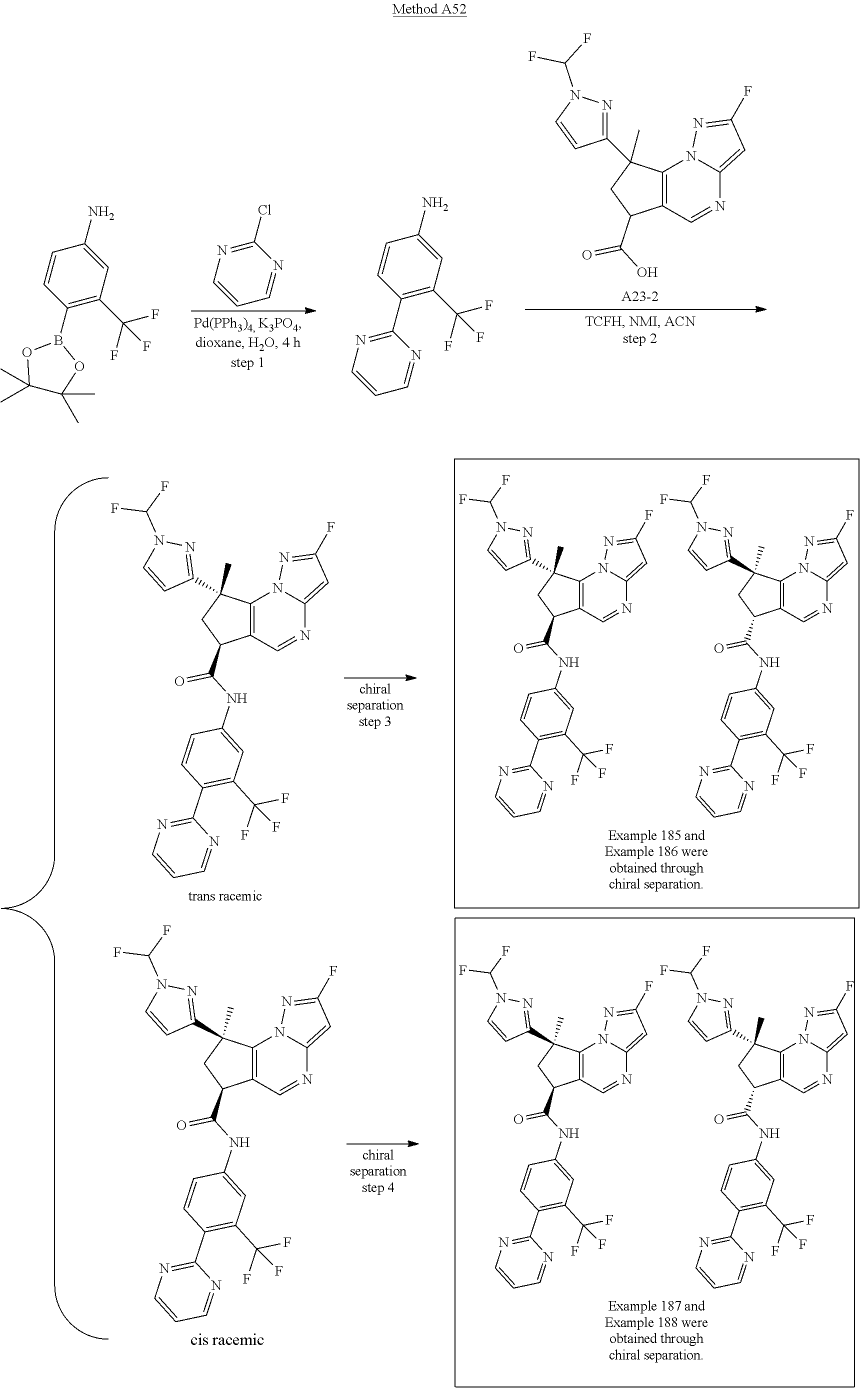

### Examples 185, 186: (6S,8S)-8-(1-(difluoromethyl)-1H-pyrazol-3-yl)-2-fluoro-8-methyl-N-(4-(pyrimidin-2-yl)-3-(trifluoromethyl)phenyl)-7,8-dihydro-6H-cyclopenta[e]pyrazolo[1,5-a]pyrimidine-6-carboxamide; (6R,8R)-8-(1-(difluoromethyl)-1H-pyrazol-3-yl)-2-fluoro-8-methyl-N-(4-(pyrimidin-2-yl)-3-(trifluoromethyl)phenyl)-7,8-dihydro-6H-cyclopenta[e]pyrazolo[1,5-a]pyrimidine-6-carboxamide

### Examples 187, 188: (6S,8R)-8-(1-(difluoromethyl)-1H-pyrazol-3-yl)-2-fluoro-8-methyl-N-(4-(pyrimidin-2-yl)-3-(trifluoromethyl)phenyl)-7,8-dihydro-6H-cyclopenta[e]pyrazolo[1,5-a]pyrimidine-6-carboxamide; (6R,8S)-8-(1-(difluoromethyl)-1H-pyrazol-3-yl)-2-fluoro-8-methyl-N-(4-(pyrimidin-2-yl)-3-(trifluoromethyl)phenyl)-7,8-dihydro-6H-cyclopenta[e]pyrazolo[1,5-a]pyrimidine-6-carboxamide

The relative and absolute stereochemistry of each example was not determined.

### Step 1: 4-(pyrimidin-2-yl)-3-(trifluoromethyl)aniline

To a stirred mixture of 4-(4,4,5,5-tetramethyl-1,3,2-dioxaborolan-2-yl)-3-(trifluoromethyl)aniline (220 mg, 766 μmol) in 1.4-dioxane (10 mL) and water (2 mL) were added 2-chloropyrimidine (105 mg, 919 mol), Pd(PPh₃)₄(88 mg, 77 mol) and tripotassium phosphate (487 mg, 2.3 mmol) at 25° C. under nitrogen atmosphere. The resulting mixture was stirred at 80° C. for 4 h under nitrogen atmosphere. The mixture was allowed to cool to 25° C., quenched with water (50 mL), and the resulting mixture was extracted with ethyl acetate (3×50 mL). The combined organic layers were dried over anhydrous sodium sulfate and concentrated under reduced pressure. The residue was purified by reverse phase column to afford the title compound (150 mg, 75% yield) as a yellow solid. LC-MS (ES, m/z): 240[M+H]⁺.

### Step 2: (trans)-8-(1-(difluoromethyl)-1H-pyrazol-3-yl)-2-fluoro-8-methyl-N-(4-(pyrimidin-2-yl)-3-(trifluoromethyl)phenyl)-7,8-dihydro-6H-cyclopenta[e]pyrazolo[1,5-a]pyrimidine-6-carboxamide and (cis)-8-(1-(difluoromethyl)-1H-pyrazol-3-yl)-2-fluoro-8-methyl-N-(4-(pyrimidin-2-yl)-3-(trifluoromethyl)phenyl)-7,8-dihydro-6H-cyclopenta[e]pyrazolo[1,5-a]pyrimidine-6-carboxamide

In analogy to Method A1 step 8 but using 8-(1-(difluoromethyl)-1H-pyrazol-3-yl)-2-fluoro-8-methyl-7,8-dihydro-6H-cyclopenta[e]pyrazolo[1,5-a]pyrimidine-6-carboxylic acid (A23-2, 50 mg, 142 mol) and 4-(pyrimidin-2-yl)-3-(trifluoromethyl)aniline (37 mg, 156 mol), racemic mixtures of the title compounds were obtained: trans isomer (25 mg, 29% yield) as a white solid, LC-MS (ES, m/z): 573[M+H]⁺; cis-isomer (15 mg, 17% yield) as a white solid, LC-MS (ES, m/z): 573[M+H]⁺.

### Step 3: Separation of Enantiomers to Obtain Example 185 and Example 186

25 mg of (trans)-8-(1-(difluoromethyl)-1H-pyrazol-3-yl)-2-fluoro-8-methyl-N-(4-(pyrimidin-2-yl)-3-(trifluoromethyl)phenyl)-7,8-dihydro-6H-cyclopenta[e]pyrazolo[1,5-a]pyrimidine-6-carboxamide were submitted to chiral HPLC purification (Column: CHIRALPAK IG, 2×25 cm, 5 m; Mobile Phase A: Hex (0.1% FA)-HPLC, Mobile Phase B: EtOH: DCM=1:1-HPLC; Flow rate: 20 mL/min; Gradient: isocratic 50% Mobile Phase B; Wave Length: 254/220 nm; RT1 (min): 3.573; RT2 (min): 6.164; Sample Solvent: EtOH-HPLC; Injection Volume: 1.1 mL; Number Of Runs: 2). The second eluting isomer was concentrated and lyophilized to afford Example 185 (8.5 mg, 32% yield) as an off-white solid. The first eluting isomer was concentrated and lyophilized to afford Example 186 (12.1 mg, 46% yield) as a white solid.

Example 185: ¹H NMR (400 MHz, DMSO-d₆) δ 10.87 (s, 1H), 8.93 (d, J=4.8 Hz, 2H), 8.71 (s, 1H), 8.28 (d, J=2.4 Hz, 1H), 8.20 (d, J=2.8 Hz, 1H), 7.98-8.01 (m, 1H), 7.80 (d, J=8.4 Hz, 1H), 7.70 (t, J=59.2 Hz, 1H), 7.53-7.56 (m, 1H), 6.62 (d, J=2.8 Hz, 1H), 6.58 (d, J=5.1 Hz, 1H), 4.49 (t, J=8.4 Hz, 1H), 2.99-3.05 (m, 1H), 2.53-2.58 (m, 1H), 2.03 (s, 3H). LC-MS (ES, m/z): 573 [M+H]⁺.

Example 186: ¹H NMR (400 MHz, DMSO-d₆) δ 10.88 (s, 1H), 8.93 (d, J=4.8 Hz, 2H), 8.71 (s, 1H), 8.28 (d, J=2.0 Hz, 1H), 8.20 (d, J=2.8 Hz, 1H), 7.98-8.02 (m, 1H), 7.80 (d, J=8.4 Hz, 1H), 7.70 (t, J=59.2 Hz, 1H), 7.53-7.56 (m, 1H), 6.62 (d, J=2.4 Hz, 1H), 6.58 (d, J=5.1 Hz, 1H), 4.49 (t, J=8.0 Hz, 1H), 2.99-3.06 (m, 1H), 2.53-2.59 (m, 1H), 2.03 (s, 3H). LC-MS (ES, m/z): 573 [M+H]⁺.

### Step 4: Separation of Enantiomers to Obtain Example 187 and Example 188

15 mg of (cis)-8-(1-(difluoromethyl)-1H-pyrazol-3-yl)-2-fluoro-8-methyl-N-(4-(pyrimidin-2-yl)-3-(trifluoromethyl)phenyl)-7,8-dihydro-6H-cyclopenta[e]pyrazolo[1,5-a]pyrimidine-6-carboxamide were submitted to chiral HPLC purification (Column: CHIRALPAK IG, 2×25 cm, 5 m; Mobile Phase A: Hex (0.1% FA)-HPLC, Mobile Phase B: EtOH: DCM=1:1-HPLC; Flow rate: 20 mL/min; Gradient: isocratic 30% Mobile Phase B; Wave Length: 254/220 nm; RT1 (min): 7.104; RT2 (min): 9.999; Sample Solvent: EtOH-HPLC; Injection Volume: 1.4 mL; Number Of Runs: 2). The second eluting isomer was concentrated and lyophilized to afford Example 187 (4.4 mg, 29% yield) as a white solid. The first eluting isomer was concentrated and lyophilized to afford Example 188 (4.0 mg, 25% yield) as a white solid.

Example 187: ¹H NMR (400 MHz, DMSO-d₆) δ 10.81 (s, 1H), 8.93 (d, J=4.8 Hz, 2H), 8.72 (s, 1H), 8.24 (d, J=2.0 Hz, 1H), 8.14 (d, J=2.4 Hz, 1H), 7.96-7.99 (m, 1H), 7.78 (d, J=8.4 Hz, 1H), 7.71 (t, J=59.2 Hz, 1H), 7.53 (t, J=4.8 Hz, 1H), 6.55 (d, J=5.1 Hz, 1H), 6.47 (d, J=2.8 Hz, 1H), 4.55 (t, J=8.0 Hz, 1H), 2.83 (d, J=8.0 Hz, 2H), 1.91 (s, 3H). LC-MS (ES, m/z): 573 [M+H]⁺.

Example 188: ¹H NMR (400 MHz, DMSO-d₆) δ 10.81 (s, 1H), 8.93 (d, J=4.8 Hz, 2H), 8.72 (s, 1H), 8.24 (d, J=2.0 Hz, 1H), 8.14 (d, J=2.4 Hz, 1H), 7.96-7.98 (m, 1H), 7.78 (d, J=8.4 Hz, 1H), 7.71 (t, J=59.2 Hz, 1H), 7.53 (t, J=4.8 Hz, 1H), 6.55 (d, J=5.1 Hz, 1H), 6.47 (d, J=2.8 Hz, 1H), 4.55 (t, J=8.0 Hz, 1H), 2.83 (d, J=8.0 Hz, 2H), 1.91 (s, 3H). LC-MS (ES, m/z): 573 [M+H]⁺.

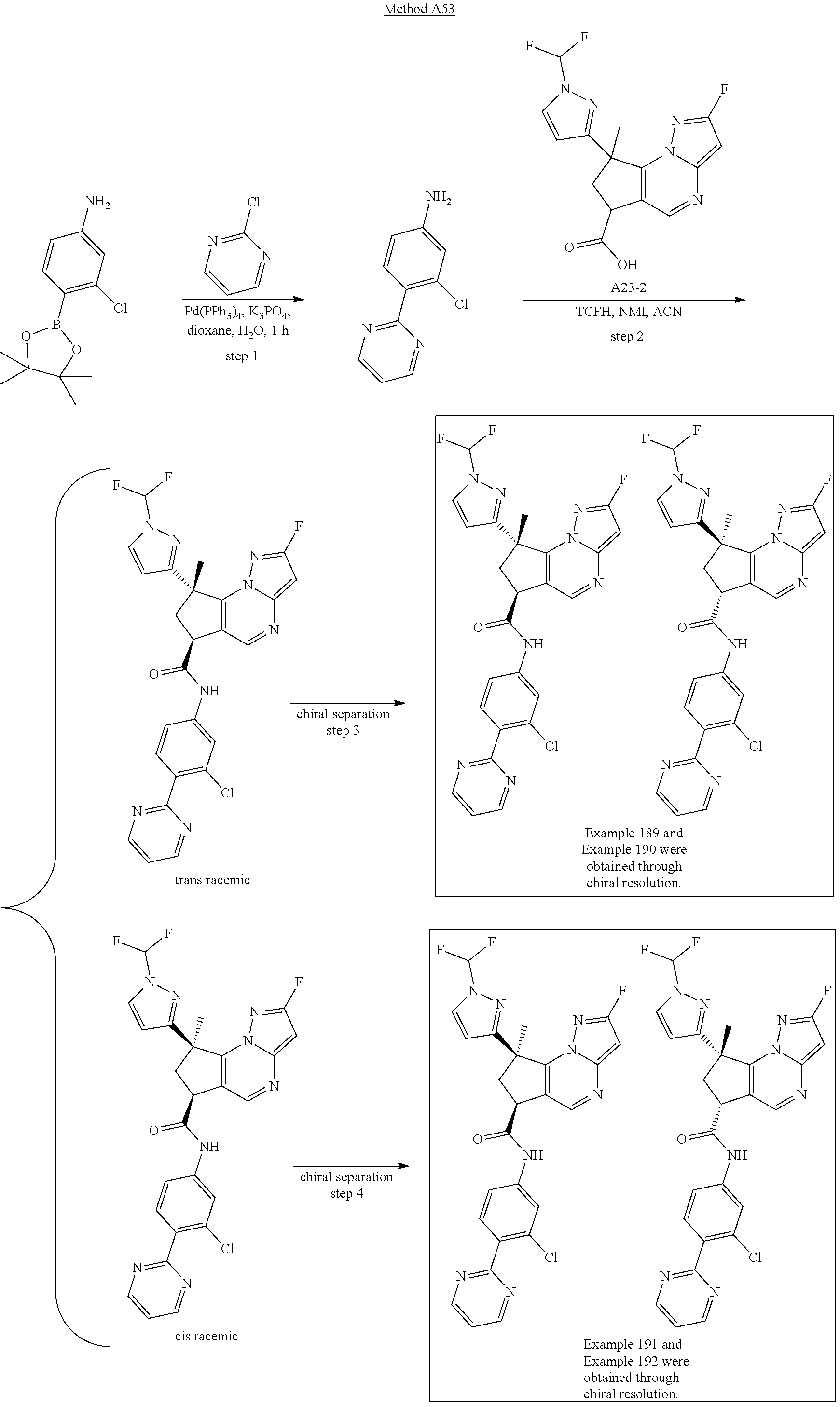

### Examples 189, 190: (6S,8S)—N-(3-chloro-4-(pyrimidin-2-yl)phenyl)-8-(1-(difluoromethyl)-1H-pyrazol-3-yl)-2-fluoro-8-methyl-7,8-dihydro-6H-cyclopenta[e]pyrazolo[1,5-a]pyrimidine-6-carboxamide; (6R,8R)—N-(3-chloro-4-(pyrimidin-2-yl)phenyl)-8-(1-(difluoromethyl)-1H-pyrazol-3-yl)-2-fluoro-8-methyl-7,8-dihydro-6H-cyclopenta[e]pyrazolo[1,5-a]pyrimidine-6-carboxamide

### Examples 191, 192: (6S,8R)—N-(3-chloro-4-(pyrimidin-2-yl)phenyl)-8-(1-(difluoromethyl)-1H-pyrazol-3-yl)-2-fluoro-8-methyl-7,8-dihydro-6H-cyclopenta[e]pyrazolo[1,5-a]pyrimidine-6-carboxamide; (6R,8S)—N-(3-chloro-4-(pyrimidin-2-yl)phenyl)-8-(1-(difluoromethyl)-1H-pyrazol-3-yl)-2-fluoro-8-methyl-7,8-dihydro-6H-cyclopenta[e]pyrazolo[1,5-a]pyrimidine-6-carboxamide

The relative and absolute stereochemistry of each example was not determined.

### Step 1: 3-chloro-4-(pyrimidin-2-yl)aniline

In analogy to Method A52 step 1 but using 3-chloro-4-(4,4,5,5-tetramethyl-1,3,2-dioxaborolan-2-yl)aniline (520 mg, 2.1 mmol), the title compound was obtained as a yellow solid (290 mg, 64% yield). LC-MS (ES. m/z): 206[M+H]⁺.

### Step 2: (trans)-N-(3-chloro-4-(pyrimidin-2-yl)phenyl)-8-(1-(difluoromethyl)-1H-pyrazol-3-yl)-2-fluoro-8-methyl-7,8-dihydro-6H-cyclopenta[e]pyrazolo[1,5-a]pyrimidine-6-carboxamide and (cis)-N-(3-chloro-4-(pyrimidin-2-yl)phenyl)-8-(1-(difluoromethyl)-1H-pyrazol-3-yl)-2-fluoro-8-methyl-7,8-dihydro-6H-cyclopenta[e]pyrazolo[1,5-a]pyrimidine-6-carboxamide

In analogy to Method A1 step 8 but using 8-(1-(difluoromethyl)-1H-pyrazol-3-yl)-2-fluoro-8-methyl-7,8-dihydro-6H-cyclopenta[e]pyrazolo[1,5-a]pyrimidine-6-carboxylic acid (A23-2, 50 mg, 142 μmol) and 3-chloro-4-(pyrimidin-2-yl)aniline (32 mg, 156 μmol), racemic mixtures of the title compounds were obtained: trans isomer (35 mg, 43% yield) as a white solid, LC-MS (ES, m/z): 539 [M+H]⁺; cis-isomer (20 mg, 24% yield) as a white solid, LC-MS (ES, m/z): 539 [M+H]⁺.

### Step 3: Separation of Enantiomers to Obtain Example 189 and Example 190

30 mg of (trans)-N-(3-chloro-4-(pyrimidin-2-yl)phenyl)-8-(1-(difluoromethyl)-1H-pyrazol-3-yl)-2-fluoro-8-methyl-7,8-dihydro-6H-cyclopenta[e]pyrazolo[1,5-a]pyrimidine-6-carboxamide were submitted to chiral HPLC purification (Column: CHIRALPAK IG, 2×25 cm, 5 μm; Mobile Phase A: Hex (0.1% FA)-HPLC, Mobile Phase B: EtOH: DCM=1:1-HPLC; Flow rate: 20 mL/min; Gradient: isocratic 60% Mobile Phase B; Wave Length: 254/220 nm; RT1 (min): 3.846; RT2 (min): 6.992; Sample Solvent: EtOH-HPLC; Injection Volume: 3.0 mL; Number Of Runs: 2). The second eluting isomer was concentrated and lyophilized to afford Example 189 (10.5 mg, 33% yield) as a white solid. The first eluting isomer was concentrated and lyophilized to afford Example 190 (15.2 mg, 48% yield) as an off-white solid.

Example 189: ¹H NMR (400 MHz, DMSO-d₆) δ 10.74 (s, 1H), 8.95 (d, J=4.8 Hz, 2H), 8.69 (s, 1H), 8.21 (d, J=2.8 Hz, 1H), 8.01 (d, J=2.0 Hz, 1H), 7.79 (d, J=8.4 Hz, 1H), 7.70 (t, J=59.2 Hz, 1H), 7.67-7.70 (m, 1H), 7.52 (t, J=4.8 Hz, 1H), 6.62 (d, J=2.8 Hz, 1H), 6.58 (d, J=4.8 Hz, 1H), 4.48 (t, J=8.0 Hz, 1H), 2.97-3.04 (m, 1H), 2.52-2.61 (m, 1H), 2.04 (s, 3H). LC-MS (ES, m/z): 539 [M+H]⁺.

Example 190: ¹H NMR (400 MHz, DMSO-d₆) δ 10.74 (s, 1H), 8.95 (d, J=4.8 Hz, 2H), 8.69 (s, 1H), 8.21 (d, J=2.4 Hz, 1H), 8.01 (d, J=2.0 Hz, 1H), 7.79 (d, J=8.4 Hz, 1H), 7.70 (t, J=59.2 Hz, 1H), 7.67-7.70 (m, 1H), 7.52 (t, J=4.8 Hz, 1H), 6.62 (d, J=2.4 Hz, 1H), 6.58 (d, J=4.8 Hz, 1H), 4.48 (t, J=8.0 Hz, 1H), 2.97-3.05 (m, 1H), 2.51-2.61 (m, 1H), 2.04 (s, 3H). LC-MS (ES, m/z): 539 [M+H]⁺.

### Step 4: Separation of Enantiomers to Obtain Example 191 and Example 192

20 mg of (cis)-N-(3-chloro-4-(pyrimidin-2-yl)phenyl)-8-(1-(difluoromethyl)-1H-pyrazol-3-yl)-2-fluoro-8-methyl-7,8-dihydro-6H-cyclopenta[e]pyrazolo[1,5-a]pyrimidine-6-carboxamide were submitted to chiral HPLC purification (Column: CHIRALPAK IG, 2×25 cm, 5 μm; Mobile Phase A: Hex (0.1% FA)-HPLC, Mobile Phase B: EtOH: DCM=1:1-HPLC; Flow rate: 20 mL/min; Gradient: isocratic 40% Mobile Phase B; Wave Length: 254/220 nm; RT1 (min): 6.883; RT2 (min): 9.731; Sample Solvent: EtOH-HPLC; Injection Volume: 2.0 mL; Number Of Runs: 2). The first eluting isomer was concentrated and lyophilized to afford Example 191 (5.2 mg, 26% yield) as a white solid. The second eluting isomer was concentrated and lyophilized to afford Example 192 (5.0 mg, 24% yield) as a white solid.

Example 191: ¹H NMR (400 MHz, DMSO-d₆) δ 10.68 (s, 1H), 8.94 (d, J=4.8 Hz, 2H), 8.71 (s, 1H), 8.15 (d, J=2.8 Hz, 1H), 7.98 (d, J=2.0 Hz, 1H), 7.78 (d, J=8.4 Hz, 1H), 7.72 (t, J=59.2 Hz, 1H), 7.64-7.68 (m, 1H), 7.51 (t, J=4.8 Hz, 1H), 6.55 (d, J=5.1 Hz, 1H), 6.46 (d, J=2.8 Hz, 1H), 4.54 (t, J=8.0 Hz, 1H), 2.79-2.84 (m, 2H), 1.91 (s, 3H). LC-MS (ES, m/z): 539 [M+H]⁺.

Example 192: ¹H NMR (400 MHz, DMSO-d₆) δ 10.68 (s, 1H), 8.94 (d, J=4.8 Hz, 2H), 8.71 (s, 1H), 8.15 (d, J=2.4 Hz, 1H), 7.98 (d, J=2.0 Hz, 1H), 7.78 (d, J=8.4 Hz, 1H), 7.72 (t, J=59.2 Hz, 1H), 7.64-7.69 (m, 1H), 7.51 (t, J=4.8 Hz, 1H), 6.55 (d, J=4.8 Hz, 1H), 6.46 (d, J=2.8 Hz, 1H), 4.54 (t, J=8.0 Hz, 1H), 2.79-2.84 (m, 2H), 1.91 (s, 3H). LC-MS (ES, m/z): 539 [M+H]⁺.

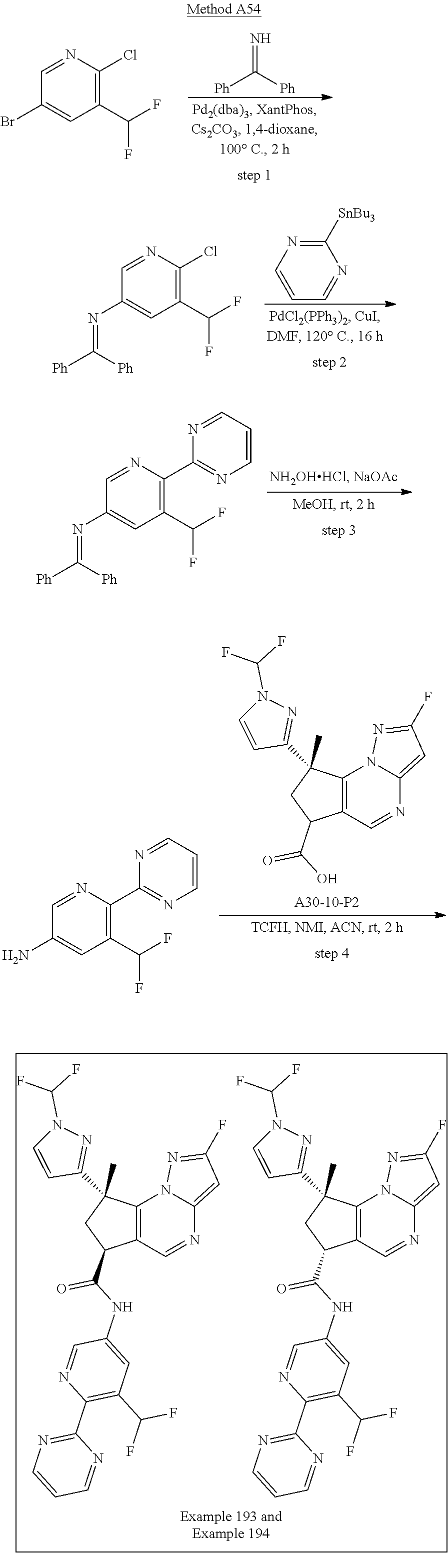

### Examples 193, 194: (6S,8S)-8-(1-(difluoromethyl)-1H-pyrazol-3-yl)-N-(5-(difluoromethyl)-6-(pyrimidin-2-yl)pyridin-3-yl)-2-fluoro-8-methyl-7,8-dihydro-6H-cyclopenta[e]pyrazolo[1,5-a]pyrimidine-6-carboxamide; (6R,8S)-8-(1-(difluoromethyl)-1H-pyrazol-3-yl)-N-(5-(difluoromethyl)-6-(pyrimidin-2-yl)pyridin-3-yl)-2-fluoro-8-methyl-7,8-dihydro-6H-cyclopenta[e]pyrazolo[1,5-a]pyrimidine-6-carboxamide

The relative and absolute stereochemistry of each example was not determined.

### Step 1: N-(6-chloro-5-(difluoromethyl)pyridin-3-yl)-1,1-diphenylmethanimine

To a solution of 5-bromo-2-chloro-3-(difluoromethyl)pyridine (1 g, 4.1 mmol) in 1,4-dioxane (10 mL) were added diphenylmethanimine (822 mg, 4.5 mmol), cesium carbonate (2.7 g, 8.3 mmol), Xantphos (477 mg, 825 μmol) and Pd₂(dba)₃ (755 mg, 825 μmol). The reaction was stirred at 100° C. for 2 h under nitrogen. The reaction was cooled to 25° C. and diluted with water (10 mL). The resulting mixture was extracted with ethyl acetate (2×10 mL). The combined organic layers were dried over anhydrous sodium sulfate and concentrated under reduced pressure. The residue was purified by silica gel column chromatography eluted with PE: EA (9:1) to afford the title compound (420 mg, 28% yield) as a yellow oil. LC-MS: m/z 343 [M+H]⁺.

### Step 2: N-(5-(difluoromethyl)-6-(pyrimidin-2-yl)pyridin-3-yl)-1,1-diphenylmethanimine

In analogy to Method A32 step 2 but using N-(6-chloro-5-(difluoromethyl)pyridin-3-yl)-1,1-diphenylmethanimine (400 mg, 1.2 mmol) and 2-(tributylstannyl)pyrimidine (2.2 g, 5.8 mmol) and stirring at 120° C. for 16 h, the title compound was obtained as a yellow oil (130 mg, 28% yield). LC-MS: m/z 387 [M+H]⁺.

### Step 3: 5-(difluoromethyl)-6-(pyrimidin-2-yl)pyridin-3-amine

To a solution of N-(5-(difluoromethyl)-6-(pyrimidin-2-yl)pyridin-3-yl)-1,1-diphenylmethanimine (120 mg, 311 μmol) in MeOH (10 mL) was added hydroxylamine hydrochloride (108 mg, 1.6 mmol) and sodium acetate (127 mg, 1.6 mmol). The reaction was stirred at 25° C. for 2 h. The reaction mixture was concentrated under reduced pressure. The residue was purified by reverse phase column to afford the title compound (25 mg, 36% yield) as a yellow solid. LC-MS: m/z 223 [M+H]⁺.

### Step 4: Example 193 and Example 194

In analogy to Method A1 step 8 but using (8S)-8-(1-(difluoromethyl)-1H-pyrazol-3-yl)-2-fluoro-8-methyl-7,8-dihydro-6H-cyclopenta[e]pyrazolo[1,5-a]pyrimidine-6-carboxylic acid (A30-10-P2, 71 mg, 202.5 μmol) and 5-(difluoromethyl)-6-(pyrimidin-2-yl)pyridin-3-amine (45 mg, 202 mol), the diastereomeric title compounds were obtained: trans isomer Example 193 (6.1 mg, 5% yield) as a white solid a cis-isomer Example 194 (13.2 mg, 11% yield) as a white solid.

Example 193: ¹H NMR (400 MHz, DMSO-d₆) δ: 11.03 (s, 1H), 9.05 (s, 1H), 8.99 (d, J=4.8 Hz, 2H), 8.74 (s, 1H), 8.63 (s, 1H), 8.21 (d, J=2.8 Hz, 1H), 7.44-7.88 (m, 3H), 6.63 (d, J=2.4 Hz, 1H), 6.59 (d, J=4.8 Hz, 1H), 4.53 (t, J=8.4 Hz, 1H), 3.00-3.08 (m, 1H), 2.54-2.62 (m, 1H), 2.04 (s, 3H). LC-MS: m/z 556 [M+H]⁺.

Example 194: ¹H NMR (400 MHz, DMSO-d₆) δ: 10.95 (s, 1H), 9.03 (s, 1H), 8.99 (d, J=4.8 Hz, 2H), 8.75 (s, 1H), 8.59 (s, 1H), 8.15 (d, J=2.4 Hz, 1H), 7.44-7.87 (m, 3H), 6.56 (d, J=4.8 Hz, 1H), 6.48 (d, J=2.4 Hz, 1H), 4.59 (t, J=8.0 Hz, 1H), 2.79-2.93 (m, 2H), 1.92 (s, 3H). LC-MS: m/z 556 [M+H]⁺.

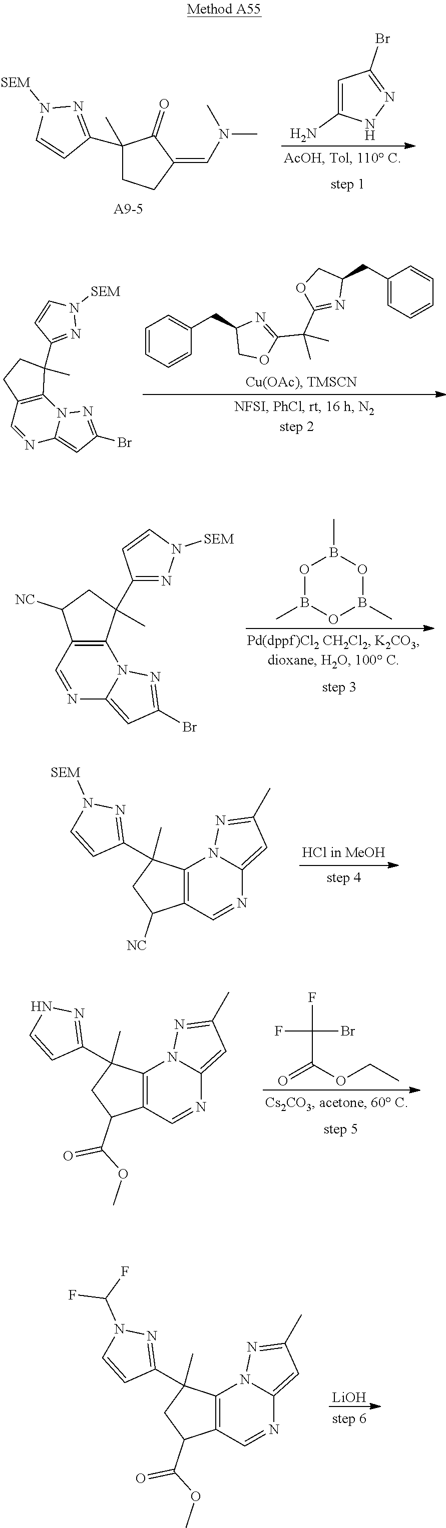

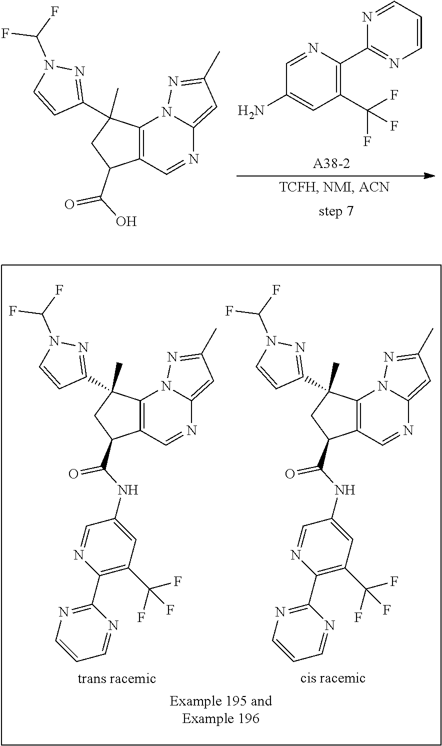

### Examples 195, 196: (tras)-8-(1-(difluoromethyl)-1H-pyrazol-3-yl)-2,8-dimethyl-N-(6-(pyrimidin-2-yl)-5-(trifluoromethyl)pyridin-3-yl)-7,8-dihydro-6H-cyclopenta[e]pyrazolo[1,5-a]pyrimidine-6-carboxamide; (cis)-8-(1-(difluoromethyl)-1H-pyrazol-3-yl)-2,8-dimethyl-N-(6-(pyrimidin-2-yl)-5-(trifluoromethyl)pyridin-3-yl)-7,8-dihydro-6H-cyclopenta[e]pyrazolo[1,5-a]pyrimidine-6-carboxamide

The relative and absolute stereochemistry of each example was not determined.

### Step 1: 2-bromo-8-methyl-8-(1-((2-(trimethylsilyl)ethoxy)methyl)-1H-pyrazol-3-yl)-7,8-dihydro-6H-cyclopenta[e]pyrazolo[1,5-a]pyrimidine

In analogy to Method A6 step 6 but using 5-((dimethylamino)methylene)-2-methyl-2-(1-((2-(trimethylsilyl)ethoxy)methyl)-1H-pyrazol-3-yl)cyclopentan-1-one (5.5 g, 15.7 mmol) and 3-bromo-1H-pyrazol-5-amine (2.6 g, 15.7 mmol) and stirring at 110° C. for 16 h, the title compound was obtained as a brown oil (4.2 g, 45% yield). LC-MS: m/z 448[M+H]⁺.

### Step 2: 2-bromo-8-methyl-8-(1-((2-(trimethylsilyl)ethoxy)methyl)-1H-pyrazol-3-yl)-7,8-dihydro-6H-cyclopenta[e]pyrazolo[1,5-a]pyrimidine-6-carbonitrile

In analogy to Method A1 step 6 but using 2-bromo-8-methyl-8-(1-((2-(trimethylsilyl)ethoxy)methyl)-1H-pyrazol-3-yl)-7,8-dihydro-6H-cyclopenta[e]pyrazolo[1,5-a]pyrimidine (4.2 g, 9.4 mmol) in Chlorobenzene (400 mL), the title compound was obtained as a light-yellow oil (2.6 g, 35% yield). LC-MS: m/z 473[M+H]⁺.

### Step 3: 2,8-dimethyl-8-(1-((2-(trimethylsilyl)ethoxy)methyl)-1H-pyrazol-3-yl)-7,8-dihydro-6H-cyclopenta[e]pyrazolo[1,5-a]pyrimidine-6-carbonitrile

To a stirred solution of 2-bromo-8-methyl-8-(1-((2-(trimethylsilyl)ethoxy)methyl)-11H-pyrazol-3-yl)-7,8-dihydro-6H-cyclopenta[e]pyrazolo[1,5-a]pyrimidine-6-carbonitrile (2.5 g, 5.3 mmol) in dioxane (80 mL) and water (20 mL) were added 2,4,6-trimethyl-1,3,5,2,4,6-trioxatriborinane (994 mg, 7.9 mmol), Pd(dppf)Cl₂·CH₂Cl₂ (431 mg, 528 mol) and potassium carbonate (2.2 g, 15.8 mmol). The reaction mixture was stirred at 100° C. for 4 h under nitrogen atmosphere. The mixture was cooled to 25° C. and concentrated under reduced pressure. The residue was diluted with water (100 mL), and it was extracted with ethyl acetate (3×100 mL). The combined organic layers were dried over anhydrous sodium sulfate and concentrated under reduced pressure. The residue was purified by reverse phase column to afford the title compound (1.1 g, 37% yield) as a brown oil. LC-MS: m/z 409[M+H]⁺.

### Step 4: Methyl 2,8-dimethyl-8-(1H-pyrazol-3-yl)-7,8-dihydro-6H-cyclopenta[e]pyrazolo[1,5-a]pyrimidine-6-carboxylate

In analogy to Method A48 step 3 but using 2,8-dimethyl-8-(1-((2-(trimethylsilyl)ethoxy)methyl)-1H-pyrazol-3-yl)-7,8-dihydro-6H-cyclopenta[e]pyrazolo[1,5-a]pyrimidine-6-carbonitrile (1.1 g, 2.6 mmol) and purification of the product by reverse phase column chromatography, the title compound was obtained as a yellow oil (250 mg, 26% yield). LC-MS: m/z 312[M+H]⁺.

### Step 5: Methyl 8-(1-(difluoromethyl)-1H-pyrazol-3-yl)-2,8-dimethyl-7,8-dihydro-6H-cyclopenta[e]pyrazolo[1,5-a]pyrimidine-6-carboxylate

In analogy to Method A23 step 1 but using methyl 2,8-dimethyl-8-(1H-pyrazol-3-yl)-7,8-dihydro-6H-cyclopenta[e]pyrazolo[1,5-a]pyrimidine-6-carboxylate (230 mg, 739 μmol) and stirring at 60° C. for 2 h, the title compound was obtained as a yellow oil (80 mg, 24% yield). LC-MS: m/z 362[M+H]⁺.

### Step 6: 8-(1-(difluoromethyl)-1H-pyrazol-3-yl)-2,8-dimethyl-7,8-dihydro-6H-cyclopenta[e]pyrazolo[1,5-a]pyrimidine-6-carboxylic acid

In analogy to Method A22 step 2 but using methyl 8-(1-(difluoromethyl)-1H-pyrazol-3-yl)-2,8-dimethyl-7,8-dihydro-6H-cyclopenta[e]pyrazolo[1,5-a]pyrimidine-6-carboxylate (80 mg, 221 μmol) the title compound was obtained as a yellow oil (40 mg, 41% yield). LC-MS: m/z 348[M+H]⁺.

### Step 7: Example 195 and Example 196

In analogy to Method A1 step 8 but using 8-(1-(difluoromethyl)-1H-pyrazol-3-yl)-2,8-dimethyl-7,8-dihydro-6H-cyclopenta[e]pyrazolo[1,5-a]pyrimidine-6-carboxylic acid (40 mg, 115 mol) and 6-(pyrimidin-2-yl)-5-(trifluoromethyl)pyridin-3-amine (A38-2, 33 mg, 138 mol), the title compounds were obtained as racemic mixtures: trans isomer Example 195 (3.2 mg, 21% yield) as an off-white solid and cis-isomer Example 196 (1.6 mg, 10.4% yield) as an off-white solid.

Example 195: ¹HNMR (400 MHz, CDCl₃) δ: 9.20 (s, 1H), 8.95 (d, J=4.8 Hz, 2H), 8.84-8.92 (m, 2H), 7.74 (s, 1H), 7.42 (t, J=4.8 Hz, 1H), 7.08 (t, J=60.4 Hz, 1H), 6.76 (s, 1H), 6.67 (s, 1H), 4.70-4.79 (m, 1H), 3.36-3.46 (m, 1H), 2.78-2.86 (m, 1H), 2.55 (s, 3H), 2.26 (s, 3H). LCMS (ES, m/z): 570 [M+H]⁺.

Example 196: ¹HNMR (400 MHz, CDCl₃) δ: 9.40 (s, 1H), 8.88-8.96 (m, 3H), 8.58 (s, 1H), 8.54 (s, 1H), 7.79 (d, J=2.8 Hz, 1H), 7.39-7.42 (m, 1H), 7.03 (d, J=2.4 Hz, 1H), 6.98 (t, J=60.8 Hz, 1H), 6.58 (s, 1H), 4.52-4.58 (m, 1H), 3.38-3.45 (m, 1H), 2.83-2.93 (m, 1H), 2.51 (s, 3H), 2.19 (s, 3H). LCMS (ES, m/z): 570 [M+H]⁺.

### Biological Assays

### MALT1 Protease Assays

MALT1 protease activity was assessed in an in vitro assay using a tetrapeptide as substrate and full-length MALT1 protein His-MALT1(1-824) purified from baculovirus-infected insect cells. The tetrapeptide substrate is Ac-LRSR-AMC (SM Biochemicals) with Kₘ measured at around 25 M.

The final assay buffer includes 1 nM of MALT1 full-length protein, 25 μM Ac-LRSR-AMC substrate, 50 mM Tris pH 7.5, 600 mM Sodium Citrate, 1 mM DTT, 1 mM EDTA, and 0.05% BSA in 384-well plate format using black microtiter square well plates (Optiplate 384-F, Perkin Elmer).

Test compounds were dissolved in 100% DMSO stock of 10 mM, with final DMSO concentration of 0.1%. Test compounds were pre-incubated with MALT1 protein for 2 h at room temperature. Substrate was added after the pre-incubation and fluorescence signal was measured using Envision at excitation 355 nm and emission 460 nm after 8 hr incubation at RT. Increase in the assay signal was linear over this period and proportional with increase in the enzyme content.

The fluorescence units were transformed to percentage of remaining activity by using the high control (HC, median of fluorescence signal from wells containing MALT1 protein, substrate, and DMSO) and low control (LC, median of fluorescence signal from wells with substrate only) with the formula below:

$$\% Remaining Activity = 100 \% \times \frac{{Signal}_{Sample} - {Signal}_{LC}}{{Signal}_{HC} - {Signal}_{LC}}$$

IC₅₀ and Hill coefficients were obtained using Graph Pad Prism (Graph Pad software, Inc, USA) with non-linear regression analysis. MALT1 inhibition IC₅₀ values of certain compounds described herein are provided in Tables A and B below.

### Human IL10 Secretion Assays

IL10 is one of the cytokines that are regulated via activation of NF-kB signaling. For example, in ABC-DLBCL cell lines, the activated NF-kB signaling leads to increased IL10 secretion. Inhibition of NF-kB signaling has been shown leading to decreased IL10 secretion.

OCI-LY10 cells were seeded in IMEM supplemented with 20% fetal bovine serum at 4.8×10⁵ cells per 160 μL per well in 96-well V-bottom cell culture plates (coming, 3894), treated with 120 nL of 3-fold serial compound dilutions, starting at 4 mM. The final vehicle concentration was 0.075% DMSO in all wells. After a 24 h incubation, human IL-10 pre-coated plates (Meso Scale Discovery) were washed 3 times with PBST, and 50 μL culture medium was aspirated into the MSD plate and incubated at 4° C. overnight. The supernatant was then discarded, and the wells were washed 3 times with PBST. SULFO-TAG Anti-human IL-10 Antibody (50×) was diluted 50-fold according to the Meso Scale Protocol, then 25 μL of SULFO-TAG Anti-human IL-10 Antibody (1×) was added. After 2 h of incubation at RT, the supernatant was discarded, and the well was washed 3 times with PBST. 2× read buffer was added and the signal was read on an MSD Sector S600. The effect of a particular compound on IL10 secretion is shown relative to the effect of DMSO; set as 100%. IC₅₀ values (nM) were determined using 4-parametric curve-fitting.

The biological activity of certain compounds using the assays described above are shown in Tables A and B. The MALT1 IC₅₀ and IL10 secretion cell assay IC₅₀ ranges are as follows: A denotes IC₅₀<10 nM; B denotes 10 nM; IC₅₀<100 nM; C denotes 100 nM≤IC₅₀<1000 nM; D denotes IC₅₀≥1000 nM. NA denotes value not determined with that assay for the specified compound.

TABLE A
IC₅₀ Values for Selected Compounds of Formula (I)

| | MALT1 | IL-10 Secretion |
|---|---|---|
| Example No. | IC₅₀ [nM] | IC₅₀ [nM] |
| 1 | A | C |
| 2 | D | NA |
| 3 | C | NA |
| 4 | D | NA |
| 5 | A | B |
| 6 | C | NA |
| 7 | C | NA |
| 8 | A | B |
| 9 | D | NA |
| 10 | C | NA |
| 11 | C | NA |
| 12 | A | C |
| 13 | D | NA |
| 14 | C | NA |
| 15 | C | NA |
| 16 | C | NA |
| 17 | D | NA |
| 18 | D | NA |
| 19 | D | NA |
| 20 | A | B |
| 21 | D | NA |
| 22 | B | NA |
| 23 | C | NA |
| 24 | A | B |
| 25 | D | NA |
| 26 | B | NA |
| 27 | D | NA |
| 28 | A | B |
| 29 | D | NA |
| 30 | B | NA |
| 31 | D | NA |
| 32 | A | C |
| 33 | D | NA |
| 34 | C | NA |
| 35 | C | NA |
| 36 | A | NA |
| 37 | D | NA |
| 38 | B | NA |
| 39 | B | NA |
| 40 | A | B |
| 41 | D | NA |
| 42 | C | NA |
| 43 | C | NA |
| 44 | B | NA |
| 45 | D | NA |
| 46 | C | NA |
| 47 | D | NA |
| 48 | A | NA |
| 49 | D | NA |
| 50 | B | NA |
| 51 | C | NA |
| 52 | B | NA |
| 53 | D | NA |
| 54 | C | NA |
| 55 | C | NA |
| 56 | B | NA |
| 57 | D | NA |
| 58 | D | NA |
| 59 | D | NA |
| 60 | B | NA |
| 61 | D | NA |
| 62 | D | NA |
| 63 | D | NA |
| 64 | B | NA |
| 65 | D | NA |
| 66 | C | NA |
| 67 | D | NA |
| 68 | A | C |
| 69 | D | NA |
| 70 | C | NA |
| 71 | D | NA |
| 72 | A | NA |
| 73 | D | NA |
| 74 | B | NA |
| 75 | C | NA |
| 76 | A | NA |
| 77 | D | NA |
| 78 | C | NA |
| 79 | C | NA |
| 80 | A | NA |
| 81 | D | NA |
| 82 | B | NA |
| 83 | C | NA |
| 84 | A | NA |
| 85 | D | NA |
| 86 | B | NA |
| 87 | C | NA |
| 88 | A | B |
| 89 | D | NA |
| 90 | B | NA |
| 91 | C | NA |
| 92 | A | C |
| 93 | D | NA |
| 94 | B | NA |
| 95 | C | NA |
| 96 | B | NA |
| 97 | D | NA |
| 98 | A | NA |
| 99 | D | NA |
| 100 | B | NA |
| 101 | C | NA |
| 102 | B | NA |
| 103 | D | NA |
| 104 | C | NA |
| 105 | C | NA |
| 106 | A | B |
| 107 | D | NA |
| 108 | C | NA |
| 109 | C | NA |
| 110 | A | B |
| 111 | D | NA |
| 112 | C | NA |
| 113 | C | NA |
| 114 | B | NA |
| 115 | C | NA |
| 116 | A | C |
| 117 | C | NA |
| 118 | B | NA |
| 119 | D | NA |
| 120 | A | B |
| 121 | D | NA |
| 122 | C | NA |
| 123 | C | NA |
| 124 | A | B |
| 125 | D | NA |
| 126 | B | NA |
| 127 | B | NA |
| 128 | B | NA |
| 129 | D | NA |
| 130 | C | NA |
| 131 | D | NA |
| 132 | B | NA |
| 133 | C | NA |
| 134 | B | NA |
| 135 | C | NA |
| 136 | A | B |
| 137 | D | NA |
| 138 | C | NA |
| 139 | C | NA |
| 140 | A | B |
| 141 | D | NA |
| 142 | D | NA |
| 143 | D | NA |
| 144 | A | C |
| 145 | D | NA |
| 146 | C | NA |
| 147 | C | NA |
| 148 | B | C |
| 149 | D | NA |
| 150 | C | NA |
| 151 | C | NA |
| 152 | A | NA |
| 153 | D | NA |
| 154 | C | NA |
| 155 | D | NA |
| 156 | B | NA |
| 157 | D | NA |
| 158 | B | NA |
| 159 | D | NA |
| 160 | C | NA |
| 161 | D | NA |
| 162 | B | NA |
| 163 | D | NA |
| 164 | A | NA |
| 165 | D | NA |
| 166 | C | NA |
| 167 | C | NA |
| 168 | B | NA |
| 169 | D | NA |
| 170 | C | NA |
| 171 | A | NA |
| 172 | D | NA |
| 173 | B | NA |
| 174 | C | NA |
| 175 | B | NA |
| 176 | D | NA |
| 177 | A | NA |
| 178 | C | NA |
| 179 | D | NA |
| 180 | C | NA |
| 181 | D | NA |
| 182 | D | NA |
| 183 | B | NA |
| 184 | D | NA |
| 185 | A | NA |
| 186 | D | NA |
| 187 | B | NA |
| 188 | C | NA |
| 189 | B | NA |
| 190 | D | NA |
| 191 | D | NA |
| 192 | C | NA |
| 193 | B | NA |
| 194 | D | NA |
| 195 | B | NA |
| 196 | C | NA |

What is claimed is:

1. A compound of Formula (I) or a pharmaceutically acceptable salt thereof: 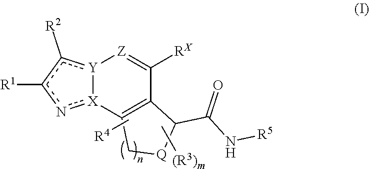
wherein
each [symbol] is a single or double bond;
Q is —CH₂—, O, or NH;
X is N or C;
Y is N or C;
Z is N or CR⁶;
wherein when one of X and Y is N, the other of X and Y is C;
n is 1, 2, or 3;
R^{X} is hydrogen or halogen;
R¹ is hydrogen, halogen, cyano, hydroxyl, C1-C3 alkoxy, C1-C3 haloalkoxy, C1-C3 haloalkyl, —NR^{A}R^{B}, or C1-C3 alkyl optionally substituted with 1-3 substituents selected from hydroxyl and C1-C3 alkoxy;
R² is hydrogen, halogen, amino, or C1-C3 alkyl;
each R³ is independently deuterium, halogen, C3-C6 cycloalkyl, C1-C3 alkyl, C1-C3 alkoxy, C1-C3 haloalkoxy, or C1-C3 haloalkyl;
m is 0, 1, 2, or 3;
R⁴ is 5-10 membered heteroaryl optionally substituted with one to three substituents each independently selected from R⁷;
R⁵ is phenyl or 5-9 membered heteroaryl, wherein each R⁵ group is optionally substituted with 1-3 substituents independently selected from R⁸;
R⁶ is hydrogen, halogen, cyano, hydroxyl, C1-C3 alkoxy, C1-C3 haloalkoxy, C1-C3 haloalkyl, —NR^{c}R^{D}, and C1-C3 alkyl;
each R⁷ is independently selected from the group consisting of halogen, cyano, hydroxyl, C1-C3 alkoxy, C1-C3 haloalkoxy, C1-C3 haloalkyl, oxetanyl, —NR^{C}R^{D}, C1-C3 alkyl optionally substituted with one cyclopropyl; and C3-C7 cycloalkyl optionally substituted with one C1-C3 alkyl;
each R¹ is independently selected from halogen; cyano; amino; —N═(S═O)(C1-C3 alkyl)₂; —S(═O)ₚ(C1-C3 alkyl); 1-imino-1-lambda⁶-thietanyl 1-oxide, —(C═O)NR^{E}R^{F}; C1-C3 alkoxy; C1-C3 haloalkyl optionally substituted with hydroxyl; C1-C3 haloalkoxy; 5-6 membered heteroaryl optionally substituted with halogen, cyano, hydroxyl, C1-C3 alkoxy, C1-C3 haloalkoxy, amino, C1-C3 haloalkyl, 4-6 membered heterocyclyl, or C1-C3 alkyl optionally substituted with hydroxyl or —NR^{E}R^{F}; C1-C4 alkyl optionally substituted with hydroxyl, —NR^{E}R^{F}, or C1-C3 alkoxy; 3-8 membered heterocyclyl; and C3-C6 cycloalkoxy;
p is 1 or 2; and
R^{A}, R^{B}, R^{C}, R^{D}, R^{E}, and R^{F}, are independently hydrogen, C1-C3 alkyl, C3-C6 cycloalkyl, or R^{A} and R^{B}, or R^{C} and R^{D}, or R^{E} and R^{F}, together with the nitrogen atom to which they are attached come together to form a 4-6 membered heterocyclyl optionally substituted with 1-2 halogens.

2. The compound of claim 1, wherein X is N and Y is C.

3. The compound of claim 1 or 2, wherein Z is N.

4. The compound of any one of claims 1-3, wherein R^{X} is hydrogen.

5. The compound of any one of claims 1-4, wherein Q is —CH₂—.

6. The compound of any one of claims 1-5, wherein R¹ is halogen.

7. The compound of any one of claims 1-6, wherein R² is hydrogen.

8. The compound of any one of claims 1-7, wherein n is 1.

9. The compound of any one of claims 1-8, wherein m is 1.

10. The compound of any one of claims 1-9, wherein each R³ is independently halogen, C3-C6 cycloalkyl, C1-C3 alkyl, C1-C3 haloalkyl, C1-C3 alkoxy, or C1-C3 haloalkoxy.

11. The compound of any one of claims 1-10, wherein m is 1 and R³ is methyl or trifluoromethyl.

12. A compound according to anyone of claims 1-11, which is a compound according to formula (Ic), or a pharmaceutically acceptable salt thereof

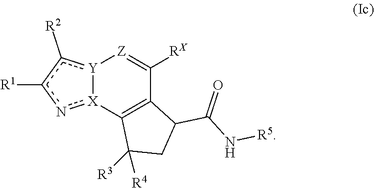

13. The compound of any one of claims 1-12, wherein R⁴ is a 5 or 6 membered heteroaryl optionally substituted with one to three substituents each independently selected from R⁷.

14. The compound of any one of claims 1-13, wherein R⁴ is a 6 membered heteroaryl optionally substituted with one to three substituents each independently selected from R⁷.

15. The compound of any one of claims 1-13, wherein R⁴ is a 5 membered heteroaryl optionally substituted with one to three substituents each independently selected from R⁷.

16. The compound of any one of claims 1-13 and 15, wherein R⁴ is 4H-pyrazolyl, 3H-pyrazolyl, triazolyl, or thiazolyl optionally substituted with one to three substituents each independently selected from R⁷.

17. The compound of any one of claims 1-16, wherein each R⁷ is independently selected from the group consisting of C1-C3 haloalkyl, C1-C3 alkyl optionally substituted with one cyclopropyl, and C3-C7 cycloalkyl optionally substituted with one C1-C3 alkyl.

18. The compound of any one of claims 1-13 and 15-17 wherein R⁴ is:

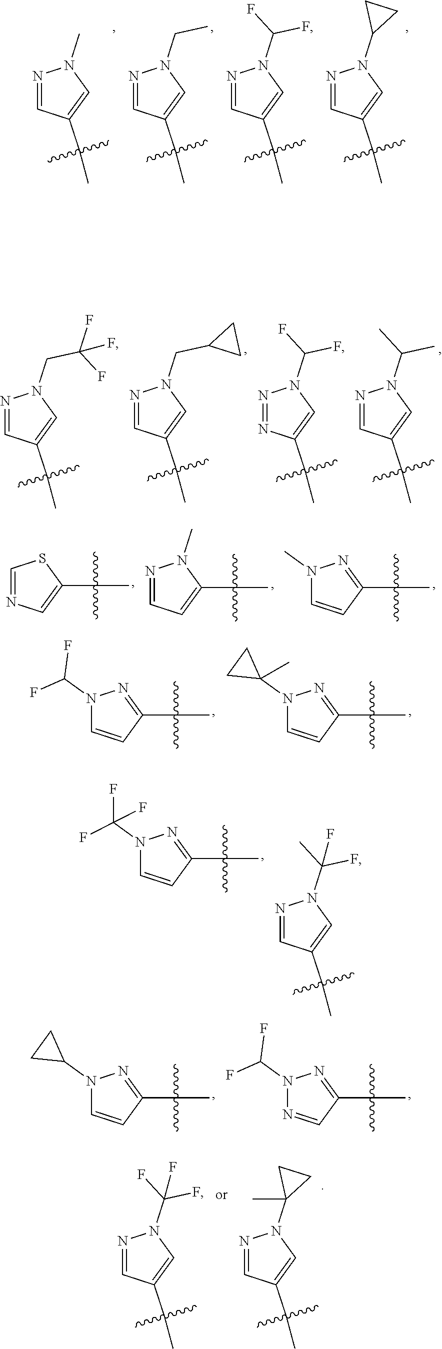

19. The compound of any one of claims 1-13, and 15-18 wherein R⁴ is:

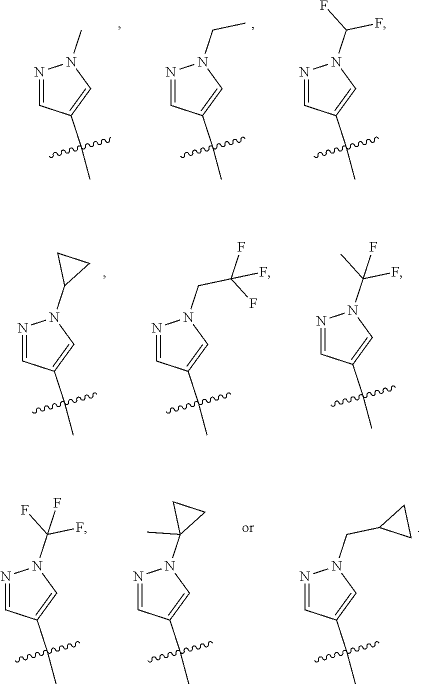

20. The compound of any one of claims 1-13 and 15-18 wherein R⁴ is:

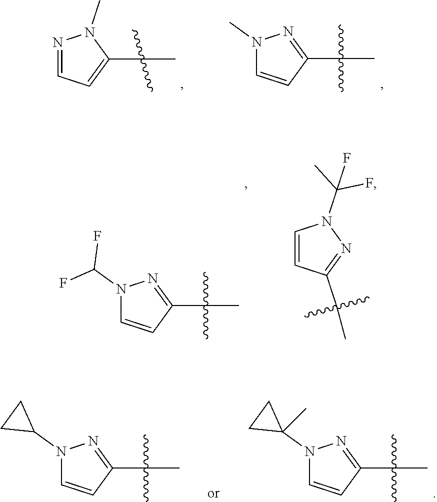

21. The compound of any one of claims 1-13 and 15-18 wherein R⁴ is:

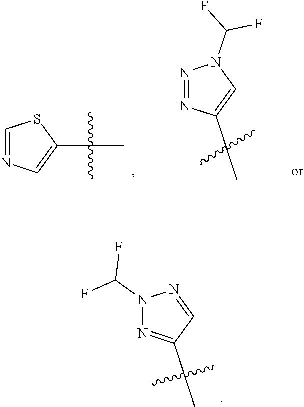 [SMILES: *c1cncs1 ; *c1cn(C(F)F)nn1 ; *c1cnn(C(F)F)n1]

22. The compound of any one of claims 1-13, 15-18 and 20 wherein R⁴ is:

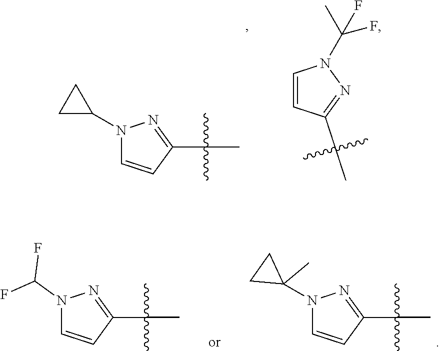

23. The compound of any one of claims 1-22, wherein R⁵ is phenyl optionally substituted with 1-3 independently selected R⁸.

24. The compound of any one of claims 1-22, wherein R⁵ is 5-9 membered heteroaryl optionally substituted with 1-3 independently selected R⁸.

25. The compound of any one of claims 1-22 and 24, wherein R⁵ is 5-6 membered heteroaryl substituted with 1-3 independently selected R⁸.

26. The compound of any one of claims 1-22, 24 and 25 wherein R⁵ is 3-pyridyl or 4-pyridyl optionally substituted with 1-3 independently selected R⁸.

27. The compound of any one of claims 1-26, wherein at least one of R⁸ is halogen.

28. The compound of any one of claims 1-27, wherein at least one of R⁸ is 5-6 membered heteroaryl optionally substituted with halogen, cyano, hydroxyl, C1-C3 alkoxy, C1-C3 haloalkoxy, C1-C3 alkyl optionally substituted with hydroxyl or —NR^{E}R^{F}, amino, or C1-C3 haloalkyl.

29. The compound of any one of claims 1-28, wherein at least one of R⁸ is 5 membered heteroaryl optionally substituted with halogen, cyano, hydroxyl, C1-C3 alkoxy, C1-C3 haloalkoxy, C1-C3 alkyl optionally substituted with hydroxyl or —NR^{E}R^{F}, amino, or C1-C3 haloalkyl.

30. The compound of any one of claims 1-26 wherein R⁵ is:

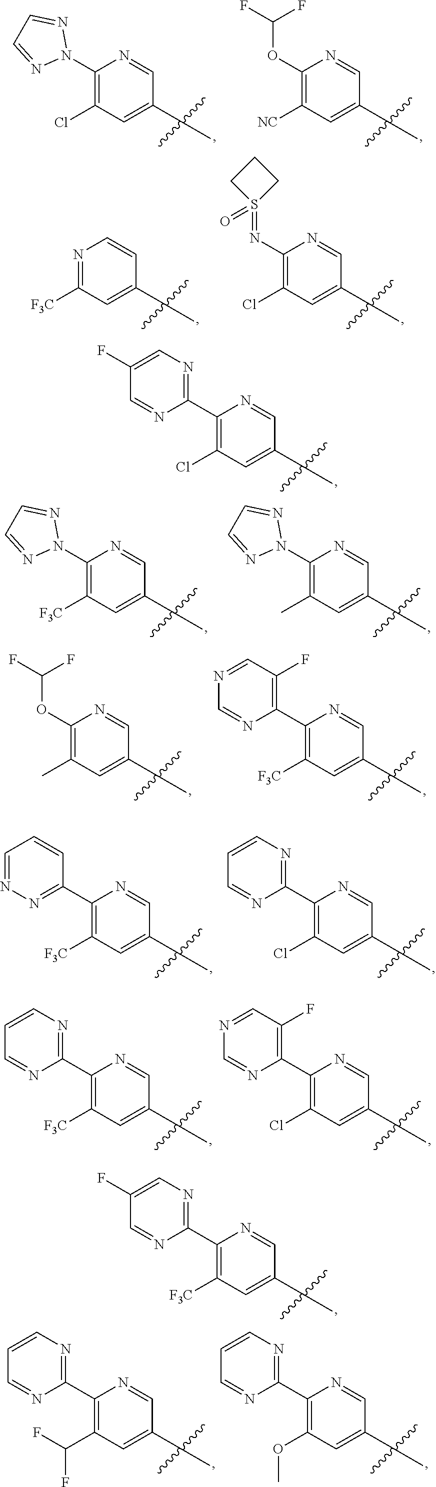

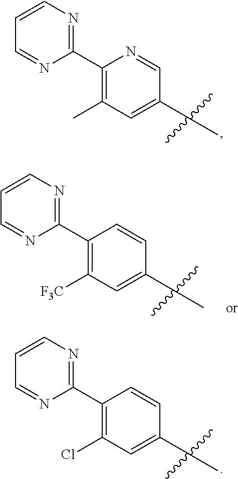 [SMILES: *c1cnc(-c2ncccn2)c(C)c1 ; *c1ccc(-c2ncccn2)c(C(F)(F)F)c1 ; *c1ccc(-c2ncccn2)c(Cl)c1]

31. The compound of any one of claims 1-26, and 30 wherein R⁵ is:

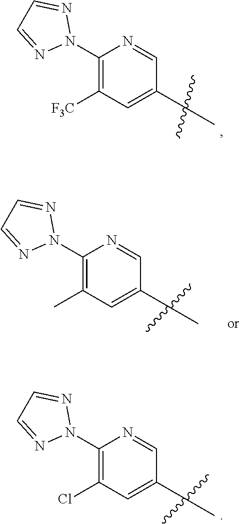 [SMILES: *c1cnc(-n2nccn2)c(C(F)(F)F)c1 ; *c1cnc(-n2nccn2)c(C)c1 ; *c1cnc(-n2nccn2)c(Cl)c1]

32. The compound of any one of claims 1-26 and 30 wherein R⁵ is:

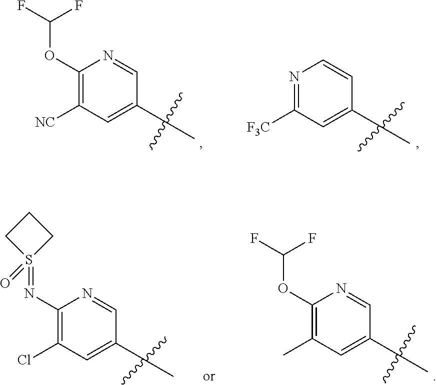 [SMILES: *c1cnc(OC(F)F)c(C#N)c1 ; *c1ccnc(C(F)(F)F)c1 ; *c1cnc(N=S2(=O)CCC2)c(Cl)c1 ; *c1cnc(OC(F)F)c(C)c1]

33. The compound of any one of claims 1-26 and 30 wherein R⁵ is:

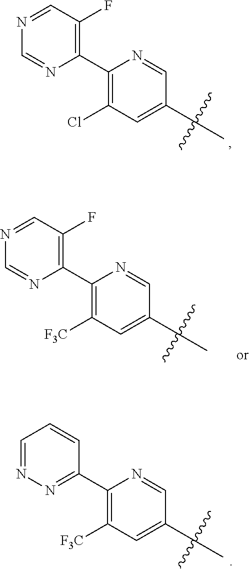 [SMILES: *c1cnc(-c2ncncc2F)c(Cl)c1 ; *c1cnc(-c2ncncc2F)c(C(F)(F)F)c1 ; *c1cnc(-c2cccnn2)c(C(F)(F)F)c1]

34. The compound of any one of claims 1-26 and 30 wherein R⁵ is:

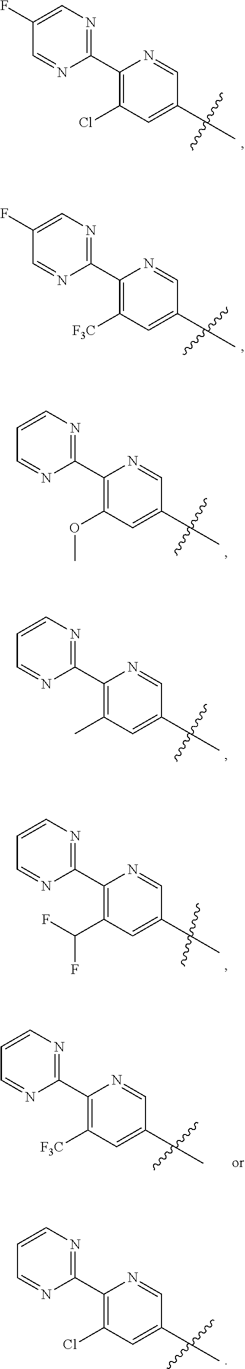

35. A compound according to anyone of claims 1-34, which is a compound according to formula (Id), or a pharmaceutically acceptable salt thereof

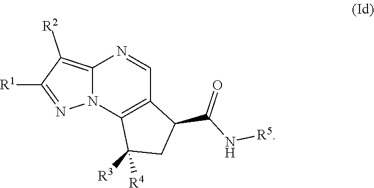

36. A compound according to anyone of claims 1-34, which is a compound according to formula (Ie), or a pharmaceutically acceptable salt thereof

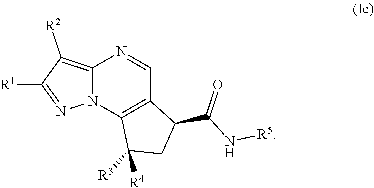

37. A compound according to anyone of claims 1-34, which is a compound according to formula (If), or a pharmaceutically acceptable salt thereof

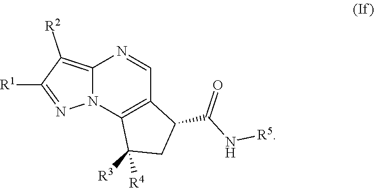

38. A compound according to anyone of claims 1-34, which is a compound according to formula (Ig), or a pharmaceutically acceptable salt thereof

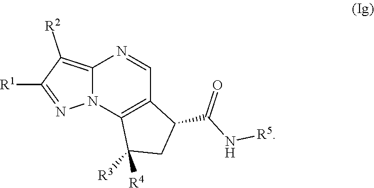

39. A compound according to claim 1, selected from Table 1, or a pharmaceutically acceptable salt thereof.

40. A pharmaceutical composition comprising a compound according to any one of claims 1-39, or a pharmaceutically acceptable salt thereof, in combination with one or more pharmaceutically acceptable excipients.

41. A method for treating cancer in a subject in need thereof, comprising administering to the subject an effective amount of a compound of any one of claims 1-39, or a pharmaceutically acceptable salt thereof, or a pharmaceutical composition of claim 40.

42. The method of claim 41 wherein the cancer is a MALT1-associated cancer.

43. The method of claims 41 or 42 wherein the cancer is lymphoma.

44. The method of claim 43 wherein the lymphoma is non-Hodgkin lymphoma, DLBCL, refractory DLBCL, (ABC) subtype of DLBCL, mantle cell lymphoma (MCL), or marginal zone lymphoma (MZL).

45. The method of claims 41 or 42 wherein the cancer is leukemia.

46. The method of claim 45 where in the leukemia is chronic lymphocytic leukemia (CLL).

47. The method of claim 41 or 42 wherein the cancer is a solid tumor.

48. A method of treating an autoimmune disorder in a subject in need thereof, comprising administering to the subject an effective amount of a compound of any one of claims 1-39, or a pharmaceutically acceptable salt thereof, or a pharmaceutical composition of claim 40.

49. The method according to claim 48 wherein the autoimmune disorder is chronic graft versus host disease.

50. The method of any one of claims 41-49, further comprising administering an additional therapy or therapeutic agent to the subject.
